(19) United States
(12) Patent Application Publication
(10) Pub. No.: US 2025/0127908 A1
(43) Pub. Date: Apr. 24, 2025

(54) HETEROBIFUNCTIONAL CONDITIONAL INHIBITORS

(71) Applicant: Kolm Therapeutics Inc., Woodbridge, CT (US)

(72) Inventors: Taavi NEKLESA, Orange, CT (US); Dominico VIGIL, Brookline, MA (US); Bruce A. LEFKER, Nahant, MA (US); Ralph P. ROBINSON, Gales Ferry, CT (US); Soumya RAY, Quincy, MA (US); Todd BOSANAC, Burlington, CT (US); Alexander W. HIRD, Belmont, MA (US)

(21) Appl. No.: 18/913,336

(22) Filed: Oct. 11, 2024

Related U.S. Application Data
(60) Provisional application No. 63/589,861, filed on Oct. 12, 2023.
(60) Provisional application No. 63/636,293, filed on Apr. 19, 2024.

(51) Int. Cl. A61K 47/55; A61K 9/00; A61K 9/08; A61K 9/20; A61K 9/48; A61K 47/64; C07D 471/04; C07D 519/00

(52) U.S. Cl. CPC A61K 47/55; A61K 9/0053; A61K 9/08; A61K 9/2013; A61K 9/2054; A61K 9/4825; A61K 9/4866; A61K 47/646; C07D 471/04; C07D 519/00

(57) ABSTRACT

Described herein are heterobifunctional small molecules, methods of making, pharmaceutical compositions and medicaments comprising such heterobifunctional small molecules, and methods of using such heterobifunctional small molecules are described herein, in the treatment of diseases and conditions, such as cancer, autoimmune diseases, and inflammatory diseases.

### CROSS-REFERENCE TO RELATED APPLICATIONS

This application claims the benefit of U.S. Provisional Patent Application No. 63/589,861, filed Oct. 12, 2023, and U.S. Provisional Patent Application No. 63/636,293, filed Apr. 19, 2024, which are incorporated herein by reference in their entireties.

### FIELD OF THE INVENTION

Described herein are heterobifunctional small molecules, methods of making, pharmaceutical compositions and medicaments comprising such heterobifunctional small molecules, and methods of using such heterobifunctional small molecules are described herein, in the treatment of diseases and conditions, such as cancer, autoimmune diseases, and inflammatory diseases.

### BACKGROUND OF THE INVENTION

Cellular homeostasis, a key hallmark of living organisms, arises from interactions between biomolecules, such as protein (e.g., enzyme) and substrate interactions, within and outside the cell. Conventionally, the function of a particular protein in a particular disease state has been investigated and controlled through the use of monofunctional molecules (e.g., an inhibitor), which occupy the active site of the disease protein, thereby forming binary complexes that inhibit or downregulate activity of the disease proteins. Such monofunctional molecules have provided a conceptual pathway toward many FDA-approved drugs as a means for treating the disease state.

However, many monofunctional molecules lack specificity for the disease proteins in question leading to undesired toxicities or lack of efficacy, or the disease proteins adapts to the monofunctional molecules thereby leading to resistance. An alternative approach with monofunctional molecules is to target the coregulator or coactivator proteins of the disease proteins. However, such coregulator or coactivator proteins are typically present in every cell, healthy or diseased, and inhibiting their activity typically leads to narrower therapeutic indices.

Described herein are heterobifunctional small molecules that engage a disease protein and a disease-dependent protein, thereby forming ternary complexes only in the disease cells that express both proteins and leading to loss of function of the disease-dependent protein. The heterobifunctional small molecules described herein comprise at least one silent binder to the disease-dependent protein and at least one binder to the disease protein. Such heterobifunctional small molecules offer anew means for treatment of diseases or conditions, with a wider therapeutic index and offer novel therapeutic targets vis a vis expansion of the disease proteins.

### SUMMARY OF THE INVENTION

In some embodiments, disclosed herein is a heterobifunctional conditional inhibitor compound of Formula (I):

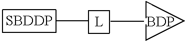

wherein:
SBDDP is a silent binder of a disease-dependent protein (DDP);
L is an optional linker; and
BDP is a binder of a disease protein (DP).

In some embodiments, disclosed herein is a compound of Formula (II):

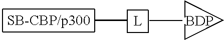

wherein:
SB-CBP/p300 is a silent binder of CBP/p300;
L is an optional linker; and
BDP is a binder of a disease protein (DP).

In some embodiments disclosed herein is a compound of Formula (III):

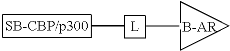

wherein:
SB-CBP/p300 is a silent binder of CBP/p300;
L is an optional linker; and
B-AR is a binder of the androgen receptor (AR).

In some embodiments, disclosed herein is a heterobifunctional compound of Formula (Ia), comprising:
a. a binder of human CREB-binding protein (CBP) or human E1A-binding protein p300 (p300) (CBP/p300);
b. a binder of a disease protein (DP); and
c. an optional linker covalently connecting a) to b);
   wherein the optional linker is covalently attached at a position of a) that is solvent exposed when a) binds to CBP/p300.

In some embodiments, disclosed herein is a stable ternary complex comprising:
a. one or more disease-dependent proteins (DDPs);
b. a disease protein (DP); and
c. heterobifunctional conditional inhibitor compound of Formula (I):

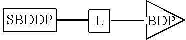

wherein:
SBDDP is a silent binder of a disease-dependent protein (DDP);
L is an optional linker; and
BDP is a binder of a disease protein (DP);
wherein DDP and DP are present in a cell of interest (COI) and the relative abundance of the DP in the COI is greater than the relative abundance of the DDP in the COI.

In some embodiments, disclosed herein is a stable ternary complex comprising:
a. CBP/p300;
b. a disease protein (DP); and
c. heterobifunctional conditional inhibitor compound of Formula (II):

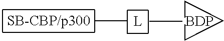

wherein:
SB-CBP/p300 is a silent binder of CBP/p300;
L is an optional linker; and
BDP is a binder of a disease protein (DP);
wherein CBP/p300 and DP are present in a cell of interest (COI) and the relative abundance of the DP in the COI is greater than the relative abundance of CBP/p300 in the COI.

In some embodiments, disclosed herein is a method of selectively inhibiting the activity of a disease-dependent protein in a cell of interest (COI) of a mammal comprising administering a heterobifunctional compound of Formula (I):

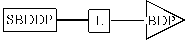

wherein:
SBDDP is a silent binder of a disease-dependent protein (DDP);
L is an optional linker; and
BDP is a binder of a disease protein (DP);
wherein the DP is overexpressed, overactive or both overexpressed and overactive in the COI; and wherein SBDDP inhibits the activity of the DDP in the COI but does not inhibit the activity of the DDP in other cells expressing the DDP and not expressing the DP.

Also described herein is a pharmaceutical composition comprising a heterobifunctional compound described herein, or a pharmaceutically acceptable salt, or solvate thereof, and at least one pharmaceutically acceptable excipient. In some embodiments, the pharmaceutical composition is formulated for administration to a mammal by oral administration, intravenous administration, or subcutaneous administration. In some embodiments, the pharmaceutical composition is formulated for administration to a mammal by oral administration. In some embodiments, the pharmaceutical composition is in the form of a tablet, a pill, a capsule, a liquid, or a suspension.

In one aspect, the heterobifunctional compounds described herein, or a pharmaceutically acceptable salt, or solvate thereof, are used in the treatment of diseases or conditions, such as cancer, autoimmune diseases, and inflammatory diseases. In some embodiments, the disease or condition is cancer. In some embodiments, the cancer comprises altered DP expression levels. In some embodiments, the cancer comprises altered androgen receptor expression levels. In some embodiments, altered DP expression levels comprises overexpressed DP, overactive DP, amplified DP, or combinations thereof.

In any of the embodiments disclosed herein, the mammal is a human. In some embodiments, compounds disclosed herein are orally administered to a human.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE FIGURES

FIG. 1 shows the ternary complex formation assay for Compound 21 and Compound 20.

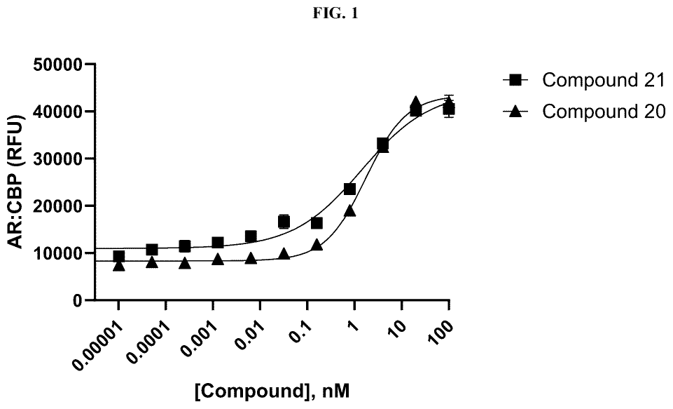

FIG. 2 shows the normalized bioluminescence resonance energy transfer (BRET) response as a function of compound concentration for GNE-781.

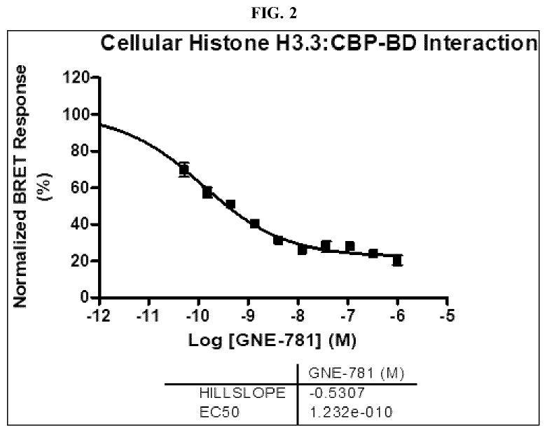

### DETAILED DESCRIPTION OF THE INVENTION

Normally, human cells grow and multiply through cell division to form new cells as the body needs them. Every cell in humans includes a collection of genes and proteins, many of which are required for survival and proliferation, and impairment of these genes and proteins leads to loss of fitness or cell death.

When cells grow old or become damaged, they die and new cells take their place. Sometimes this orderly process breaks down, and abnormal or damaged cells grow and multiply when they shouldn't. In the context of disease states, such as cancerous, autoimmune, or inflammatory states, certain proteins drive the disorderly growth and multiplication of abnormal or damaged cells.

Conventional monofunctional molecules (e.g., activators, inhibitors) form binary complexes with a target protein. In these binary complexes, the activators or inhibitors target a functional site, either orthosterically or allosterically, to modulate the target protein.

Many desired cellular changes cannot be accomplished through inhibition alone. Another class of molecules, i.e., bifunctional molecules, have been developed that operate by inducing proximity between the target proteins to form ternary complexes, which evokes a number of functions beyond inhibition.

Bifunctional molecules have primarily been utilized for their potential to simultaneously engage two macromolecular targets to form ternary complexes that in turn result in new and unique biological and cellular activities. The bifunctional molecules have seen promise in the applications of chemical induced dimerization, the inhibition of protein-protein interactions (PPIs), the degradation of target proteins, the simultaneous catalysis of two or more enzymatic processes, and the promotion or inhibition of protein aggregation. However, bifunctional molecules are not limited to the simultaneous engagement of targets. These molecules can be rationally designed from two functional chemical moieties for various dual functions such as the dual inhibition of synergistic proteins in diseases, targeted drug delivery, and activity-based profiling. Such applications have been proposed to replace combination therapies.

In ternary complexes, the bifunctional molecules can bind to various sites, including active or allosteric sites. While conventional inhibitors are occupancy driven, bifunctional molecules are often event driven. As a result, conventional inhibitors are stoichiometric, while bifunctional molecules can be sub-stoichiometric and catalytic. Furthermore, conventional inhibitors require strong binding affinities, whereas bifunctional molecules may exhibit low-to-moderate binding affinities to targeted proteins, as some ternary complexes rely on cooperativity. Compared with protein inhibitors, which can globally affect protein targets, these bifunctional molecules can be used to localize enzymatic activity to a given target. In addition, binary complexes have a saturation binding effect, where at high concentrations, the binding site is occupied. In contrast, ternary complexes can exhibit a hook effect, where high concentrations of the small molecule can saturate the two binding partners into individual binary complexes, resulting in loss of efficacy at a higher dose. Mathematical frameworks to describe the three-body equilibria have been developed to support experimental and theoretical findings of these ternary complexes (E. F. Douglass Jr., et al., A comprehensive mathematical model for three-body binding equilibria, J. Am. Chem. Soc., 135 (2013), pp. 6092-6099).

Cancer cells show extensive alterations in protein expression levels, which are drivers of their malignant transformation. Proteins with altered expression levels in cancer are involved in protein synthesis and degradation, signaling and metabolic pathways, DNA repair, apoptosis, and other cellular processes, whose alterations cause tumor development and progression.

A key component of successful drug development is the assessment of the therapeutic index (TI), the ratio of the dose or exposure of a drug required to elicit the desired therapeutic effect compared with the dose or exposure at which toxicity becomes limiting. While drugs with a high TI effectively kill cancer cells with manageable toxicities, drugs with a low TI cause significant side effects at or below efficacious doses. Cytotoxic chemotherapies, which typically target proliferating cells, generally have low TIs.

The modulation of disease protein levels is an effective anticancer target, which is achieved by targeting of up-regulated proteins in cancer, such as, but not limited to, androgen receptor, estrogen receptor, epidermal growth factor receptor 2 (HER2), and vascular endothelial growth factor (VEGF). The development of targeted therapeutics, typically monofunctional molecules, has provided alternative routes to achieving high TIs by either targeting cancer dysregulated genes with limited requirements for homeostasis in adults (e.g., ABL, KIT, TRK, ALK), or by developing mutation-biased inhibitors (e.g., KRAS^{G12C}).

However, targeting proteins that are required for survival and proliferation of cancer cells (i.e., disease-dependent proteins), such as cell cycle regulators, mitotic kinases, and epigenetic regulators, results in cellular loss of fitness or cell death of cancer cells, but the same proteins are also targeted in healthy cells. The result is a low TI drug that either fails approval by regulatory agencies or has limited use in treatment.

Described herein is a novel treatment modality for treating cancers, and other disease states. The novel heterobifunctional compounds described herein comprise at least two different monofunctional compounds, one that targets a disease protein and the other that targets a disease-dependent protein, with an optional linker connecting the two together. The targeting of the disease-dependent protein is achieved with a silent binder, such that the binding of the silent binder to the disease-dependent protein in the diseased cell (i.e., cancer cell) or healthy cells leads to minimal or no inhibition of the disease-dependent protein. When the binder of the disease protein binds to its target and the silent binder binds with its target, inhibition of disease-dependent protein is the result. The novel heterobifunctional compounds described herein are therapeutics with a high TI.

Protein pairs for the heterobifunctional compounds described herein can be chosen from resources such as, but not limited to: The Cancer Genome Atlas (TCGA), TCGA Pan-Cancer project, The Cancer Cell Line Encyclopedia (CCLE) consortium, the Genomics of Drug Sensitivity in Cancer (GDSC), Clinical Proteomic Tumour Analysis Consortium (CPTAC), Protein Interaction Network Analysis (PINA) platform, protein-protein interaction (PPI) network based on computational methods have been used to identify disease-specific genes, modules, and cancer-subtype subnetworks (Kann M G. Protein interactions and disease: computational approaches to uncover the etiology of diseases. Brief Bioinform. 2007; 8:333-46), Proteomics Database (PD), Dependency Map (DepMap), and Protein Abundance Database (PAXdb).

### Heterobifunctional Inhibitors

In some embodiments disclosed herein is a heterobifunctional inhibitor. In some embodiments, the heterobifunctional inhibitor is a heterobifunctional conditional inhibitor.

In some embodiments, the heterobifunctional conditional inhibitor compound is a compound of Formula (I):

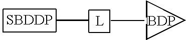

wherein: SB is a silent binder of a disease-dependent protein (DDP); L is an optional linker; and BDP is a binder of a disease protein (DP).

In some embodiments disclosed herein is a stable ternary complex comprising:
a. one or more disease-dependent proteins (DDPs);
b. a disease protein (DP); and
c. heterobifunctional conditional inhibitor compound of Formula (I):

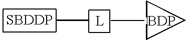

wherein: SBDDP is a silent binder of a disease-dependent protein (DDP); L is an optional linker; and BDP is a binder of a disease protein (DP); wherein DDP and DP are present in a cell of interest (COI) and the relative abundance of the DP in the COI is greater than the relative abundance of the DDP in the COI

In some embodiments disclosed herein is a stable ternary complex comprising:
a. CBP/p300;
b. a disease protein (DP); and
c. heterobifunctional conditional inhibitor compound of Formula (II):

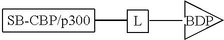

wherein: SB-CBP/p300 is a silent binder of CBP/p300; L is an optional linker; and BDP is a binder of a disease protein (DP); wherein CBP/p300 and DP are present in a cell of interest (COI) and the relative abundance of the DP in the COI is greater than the relative abundance of CBP/p300 in the COI.

In some embodiments disclosed herein is a method of selectively inhibiting the activity of a disease-dependent protein in a cell of interest (COI) of a mammal comprising administering a heterobifunctional compound of Formula (I):

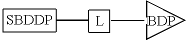

wherein:
SBDDP is a silent binder of a disease-dependent protein (DDP); L is an optional linker; and BDP is a binder of a disease protein (DP); wherein the DP is overexpressed, overactive or both overexpressed and overactive in the COI; and wherein SBDDP binds to the DDP and inhibits the activity of the DDP in the COI if the BDP simultaneously binds to the DP.

In some embodiments disclosed herein is a compound of Formula (III):

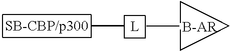

wherein:
SB-CBP/p300 is a silent binder of CBP/p300;
L is an optional linker; and
B-AR is a binder of the androgen receptor (AR).

In some embodiments, disclosed herein is a heterobifunctional compound of Formula (Ia), comprising:
a. a binder of human CREB-binding protein (CBP) or human E1A-binding protein p300 (p300) (CBP/p300);
b. a binder of a disease protein (DP); and
c. an optional linker covalently connecting a) to b);
   wherein the optional linker is covalently attached at a position of a) that is solvent exposed when a) binds to CBP/p300.

In some embodiments, wherein SBDDP binds to the DDP and inhibits the activity of the DDP in the COI if the BDP simultaneously binds to the DP and the relative abundance of the DP in the COI is greater than the DDP in the COI.

In some embodiments, BDP is a non-silent binder or silent binder of a disease protein (DP). In some embodiments, BDP is a non-silent binder of a DP. In some embodiments, BDP is a silent binder of a disease protein DP.

In some embodiments, DDP and DP are both expressed in a cell of interest (COI). In some embodiments, CBP/p300 and DP are both expressed in a cell of interest (COI). In some embodiments, CBP/p300 and AR are both expressed in a cell of interest (COI).

In some embodiments, the relative abundance of the DP in the COI is greater than the relative abundance of the DDP in the COI. In some embodiments, the relative abundance of the DP in the COI is greater than the relative abundance of the DDP in the COI by a factor of at least about 2, at least about 5, at least about 10, at least about 50, at least about 100, or about least about 250. In some embodiments, the relative abundance of the DP in the COI is greater than the relative abundance of the DDP in the COI by a factor of at least about 100. In some embodiments, the relative abundance of the DP in the COI is greater than the relative abundance of CBP/p300 in the COI. In some embodiments, the relative abundance of the DP in the COI is greater than the relative abundance of CBP/p300 in the COI by a factor of at least about 2, at least about 5, at least about 10, at least about 50, at least about 100, or about least about 250. In some embodiments, the relative abundance of the DP in the COI is greater than the relative abundance of CBP/p300 in the COI by a factor of at least about 100. In some embodiments, the relative abundance of AR in the COI is greater than the relative abundance of the CBP/p300 in the COI. In some embodiments, the relative abundance of AR in the COI is greater than the relative abundance of CBP/p300 in the COI by a factor of at least about 2, at least about 5, at least about 10, at least about 50, at least about 100, or about least about 250. In some embodiments, the relative abundance of AR in the COI is greater than the relative abundance of CBP/p300 in the COI by a factor of at least about 100.

In some embodiments, the COI is a diseased cell, and the DP is overexpressed, overactive, or both overexpressed and overactive, or amplified in the diseased cell as compared to when the COI is a non-diseased cell. In some embodiments, the COI is a diseased cell, and the DP is overexpressed in the diseased cell as compared to when the COI is a non-diseased cell. In some embodiments, the COI is a diseased cell, and the DP is overactive in the diseased cell as compared to when the COI is a non-diseased cell. In some embodiments, the COI is a diseased cell, and the DP is overexpressed and overactive in the diseased cell as compared to when the COI is a non-diseased cell. In some embodiments, the COI is a diseased cell, and the DP is amplified in the diseased cell as compared to when the COI is a non-diseased cell. In some embodiments, COI is a diseased cell, and AR is overexpressed, overactive, or both overexpressed and overactive, or amplified in the diseased cell as compared to when the COI is a non-diseased cell.

In some embodiments, the activity of the DDP is reduced or inhibited by the compound of Formula (I), (II), or (III) when the DDP and DP are both expressed in the same COI and the relative abundance of the DP in the COI is greater than the relative abundance of the DDP in the COI. In some embodiments, the activity of the DDP is reduced by the compound of Formula (I), (II), or (III) when the DDP and DP are both expressed in the same COI and the relative abundance of the DP in the COI is greater than the relative abundance of the DDP in the COI. In some embodiments, the activity of the DDP is inhibited by the compound of Formula (I), (II), or (III) when the DDP and DP are both expressed in the same COI and the relative abundance of the DP in the COI is greater than the relative abundance of the DDP in the COI.

In some embodiments, the activity of the CBP/p300 is reduced or inhibited by the compound of Formula (Ia) when the CBP/p300 and DP are both expressed in the same COI and the relative abundance of the DP in the COI is greater than the relative abundance of the CBP/p300 in the COI.

In some embodiments, the activity of CBP/p300 is reduced or inhibited by the compound of Formula (III) when CBP/p300 and AR are both expressed in the same COI and the relative abundance of the AR in the COI is greater than the relative abundance of the CBP/p300 in the COI.

In some embodiments, the activity of the DDP is unaltered by the compound of Formula (I), (II), or (III) when the DDP and DP are not both expressed in the same COI and/or the relative abundance of the DP in the COI is not greater than the relative abundance of the DDP in the COI.

In some embodiments, the activity of the CBP/p300 is unaltered by the compound of Formula (Ia) when the CBP/p300 and DP are not both expressed in the same COI and/or the relative abundance of the DP in the COI is not greater than the relative abundance of the CBP/p300 in the COI.

In some embodiments, the activity of CBP/p300 is unaltered by the compound of Formula (III) when CBP/p300 and AR are not both expressed in the same COI and/or the relative abundance of the AR in the COI is not greater than the relative abundance of the CBP/p300 in the COI.

In some embodiments, the DP is the androgen receptor (AR) and the BDP is an AR binder. In some embodiments, the DP is the androgen receptor (AR) and the BDP is an AR antagonist or agonist. In some embodiments, the DP is the androgen receptor (AR) and the BDP is an AR antagonist comprising a N-(3-(3-chloro-4-cyanophenoxy)-2,2,4,4-tetramethylcyclobutyl)-aryl-carboxamide or a N-(3-(3-trifluroromethyl-4-cyanophenoxy)-2,2,4,4-tetramethylcyclobutyl)-aryl-carboxamide. In some embodiments, the DP is the androgen receptor (AR) and the BDP is an AR antagonist that is flutamide, hydroxylflutamide, nilutamide, bicalutamide, enzalutamide, apalutamide, ODM201, AZD3514, BMS641988, or N-[trans-3-(3-Chloro-4-cyanophenoxy)-2,2,4,4-tetramethylcyclobutyl]-1-(2-hydroxyethyl)-1H-pyrazole-4-carboxamide.

### Cell of Interest (COI)

In some embodiments, the COI is a diseased cell where inhibition of disease-dependent protein (DDP) is desirable. In some embodiments, the COI is a diseased cell. In some embodiments, the cell of interest is a cancer cell. In some embodiments, the cancer cell is associated with a carcinoma, sarcoma, leukemia, lymphoma, myeloma, or the central nervous system. In some embodiments, the cancer cell is associated with a carcinoma, for example, squamous cell carcinoma, adenocarcinoma, transitional cell carcinoma, or basal cell carcinoma. In some embodiments, the cancer cell is an epithelial cell, for example, a squamous cell, adenomatous cell, transitional cell, or basal cell. In some embodiments, the cancer cell is associated with a sarcoma, for example, bone sarcoma or soft tissue sarcoma. In some embodiments, the cancer cell is a bone cell, cartilage cell, or muscle cell. In some embodiments, the cancer cell is associated with a leukemia. In some embodiments, the cancer cell is a white blood cell. In some embodiments, the cancer cell is associated with a lymphoma or myeloma. In some embodiments, the cancer cell is a white blood cell or plasma cell. In some embodiments, the cancer cell is associated with the central nervous system, for example, the brain or spinal cord. In some embodiments, the cancer cell is a glial cell.

In some embodiments, the COI is a cell associated with head and neck cancer, laryngeal and hypopharyngeal cancer, nasal cavity and paranasal sinuses cancer, nasopharyngeal cancer, oral cavity (mouth) and oropharyngeal (throat) cancer, or salivary gland cancer. In some embodiments, the COI is a cell associated with anal cancer, bile duct cancer, colorectal cancer, esophagus cancer, gallbladder cancer, gastrointestinal neuroendocrine tumors, gastrointestinal stromal tumor, liver cancer pancreatic cancer, pancreatic neuroendocrine tumor, small intestine cancer, or stomach cancer. In some embodiments, the COI is a cell associated with associated with bladder cancer, kidney cancer, or Wilms tumor. In some embodiments, the COI is a cell associated with lung cancer, lung carcinoid tumor, or malignant mesothelioma. In some embodiments, the COI is a cell associated with breast cancer. In some embodiments, the COI is a cell associated with cervical cancer, endometrial cancer, ovarian cancer, penile cancer, prostate cancer, testicular cancer, uterine sarcoma, vaginal cancer, or vulvar cancer. In some embodiments, the COI is a cell associated with adrenal cancer, gastrointestinal neuroendocrine tumors, lung carcinoid tumor, pancreatic neuroendocrine tumor, pituitary tumors, or thyroid cancer. In some embodiments, the COI is a cell associated with skin cancer, basal and squamous cell skin cancer, Kaposi sarcoma, lymphoma of the skin, melanoma skin cancer, or Merkel cell skin cancer. In some embodiments, the COI is a cell associated with bone cancer, Ewing family of tumors, osteosarcoma, rhabdomyosarcoma, or soft tissue sarcoma. In some embodiments, the COI is a cell associated with eye cancer or retinoblastoma. In some embodiments, the COI is a cell associated with brain and spinal cord tumors or neuroblastoma. In some embodiments, the COI is a cell associated with leukemia, acute lymphocytic leukemia, acute myeloid leukemia, chronic lymphocytic leukemia, chronic myeloid leukemia, chronic myelomonocytic leukemia, lymphoma, non-Hodgkin lymphoma, Hodgkin-lymphoma, multiple myeloma, myelodysplastic syndromes, thymus cancer, or Waldenstrom macroglobulinemia.

In some embodiments, the COI is a cell associated with prostate cancer or breast cancer. In some embodiments, the COI is a cell associated with prostate cancer, luminal breast cancer, and luminal androgen receptor triple-negative breast cancer.

### Disease-Dependent Protein (DDP)

“Disease-dependent protein” or “DDP” refers to any one of the proteins expressed in cell of interest that is/are required for cell functioning and/or maintenance and/or survival in a particular disease. That is, the indicated disease is dependent on the disease-dependent protein to survive. In some embodiments, a cell of interest includes one type of DDP. In some embodiments, a cell of interest expresses more than one type of DDPs, and each type of DDPs performs functions distinct from any other type of DDPs in the cell of interest. In some embodiments, a cell of interest expresses more than one type of DDPs, and each type of DPPs performs functions substantially the same or overlapping with at least one other type of DDPs in the cell of interest.

In some embodiments, the disease-dependent protein (DDP) is AT-Hook containing transcription factor 1 (AHCTF1), Anaphase promoting complex subunit 1 (ANAPC1), ANAPC4, ANAPC5, Ataxia-telangiectasia mutated (ATM), Ataxia telangiectasia and Rad3-related protein (ATR), Aurora Kinase A (AurkA), Aurora Kinase B (AurkB), Bromodomain-containing protein 4 (BRD4), Bromodomain PHD finger transcription factor (BPTF), BUB1 mitotic checkpoint serine/threonine kinase B (BUB1B), CREB-binding protein (CBP)/p300, Cell division cycle 7-related protein kinase (CDC7), CDC16, CDC23, CDC27, CDC45, Centromere protein W (CENPW), Chromatin assembly factor 1 subunit b (CHAF1B), Chromodomain helicase DNA binding protein 4 (CHD4), Checkpoint kinase 1 (CHK1), CHK2, Cleavage factor polyribonucleotide kinase subunit 1 (CLP1), CWC22, Cyclin-dependent kinase 1 (CDK1), CDK2, CDK4, CDK5, CDK6, CDK9, CDK11a, Cytoskeleton associated protein 5 (CKAP5), DDB1 and CUL4 associated factor 1 (DCAF1), DEAD-box helicase 1 (DBR1), DDX10, DDX41, DDX47, DDX54, DDX56, Dehydrodolichyl diphosphate synthase subunit (DHDDS), Deoxyhypusine synthase (DHPS), DEAH-box helicase 8 (DHX8), DONSON, DNA methyltransferase 1 (DNMT1), Denticleless E3 ubiquitin protein ligase homolog (DTL), histone acetyltransferase P300 (EP300), E1A binding protein P400 (EP400), Extra spindle pole bodies like 1, separase (ESPL1)/separin, F-Box Protein 5 (FBXO5), Gem nuclear organelle associated protein 5 (GEMINS), GINS2, GPN-Loop GTPase 3 (GPN3), HAUS augmin like complex subunit 1 (HAUS1), HAUS6, Host cell factor C1 (HCFC1), Histone deacetylase 1 (HDAC1), HDAC2, HDAC3, HEAT repeat containing 1 (HEATRI), Integrator complex subunit 11 (INTS11), Lysine acetyltransferase 8 (KAT8)/MYST1, Kinesin family member 11 (KIF11), KIF18A, KIF23, Mitotic arrest deficient 2 like 1 (MAD2A/MAD2L1), MAK16, Microtubule associate serine/threonine kinase like (MASTL), Midasin AAA ATPase 1 (MDN1), Mediator complex subunit 14 (MED14), MED11, MED28, Mitogen-activated protein kinase 1 (MEK1), MEK2, MIS18A, Methyl methanesulfonate-sensitivity protein 22-like (MMS22L), Myc, Mammalian target of rapamycin (MTOR), Neuronal precursor cell-expressed developmentally down-regulated protein 8 (NEDD8), NIP7, Nucleolar complex associated 4 homolog (NOC4L), Nucleolar protein 6 (NOL6), Notchless homolog 1 (NLE1), NUF2, Nucleoporin 205 (NUP205), NUS1, Opa interacting protein 5 (OIP5), Programmed cell death 11 (PDCD11), Phosphatidylinositol-3 kinase (PI3K), Polo-like kinase 1 (PLK1), DNA polymerase alpha 1, catalytic subunit (POLA1), DNA polymerase delta 1, catalytic subunit (POLD1), DNA polymerase epsilon, catalytic subunit (POLE), DNA polymerase epsilon, subunit 2 (POLE2), Peptidylprolyl isomerase domain and WD repeat containing 1 (PPWD1), Protein regulator of cytokinesis 1 (PRC1), DNA primase subunit 1 (PRIM1)/DNA PRIMASE, Protein arginine methyltransferase 5 (PRMT5), 20S proteasome subunits, RAD51, Rac GTPase activating protein 1 (RACGAP1), Ribonucleoside-diphosphate reductase subunit M1 (RRMI1), Ribonucleic acid export 1 (RAE1), RNA polymerase I subunit B (POLR1B), RNA polymerase II subunit J (POLR2J), RNA polymerase II associated protein 1 (RPAP1), RNA guanylyltransferase and 5′-phosphatase (RNGTT), Splicing factor 3b subunit 1 (SF3B1), SWI/SNF related, matrix associated, actin dependent regulator of chromatin, subfamily A, member 2/4 (SMARCA2/4), Small nuclear RNA activating complex polypeptide 5 (SNAPC5), Structural maintenance of chromosomes 4 (SMC4), SMG1, SGO1, Steroid receptor coactivator 1 (SRC1), SRC2, SRC3, SLU7, SPC24, SPT6H, SPT5H, Symplekin scaffold protein (SYMPK), TIMELESS, Thioredoxin like 4a (TXNL4A), Tonsoku-like, DNA repair protein (TONSL), Topoisomerase II alpha (TOP2A), TPX2, Transformation/transcription domain associated protein (TRRAP), Trafficking protein particle complex subunit 8 (TRAPPC8), TSR2, Ubiquitously expressed transcript protein (UXT), URB1, UTP15, UTP20, Ubiquitin specific peptidase like 1 (USPL1), Vacuolar protein-sorting-associated protein 25 (VPS25), WD repeat-containing protein 3 (WDR3), WDR5, WDR12, WDR43, WDR46, WDR70, WD74, WEE1, WW domain binding protein 11 (WBP11), Xeroderma pigmentosum group A-binding protein 2 (XAB2), or Exportin 1 (XPO1).

In some embodiments, the disease-dependent protein (DDP) is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2, SMARCA4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3. In some embodiments, DDP is Aurora Kinase A (AurkA), Checkpoint kinase 1 (CHK1), CHK2, CDK4, CDK6, Myc, SMARCA2, SMARCA4, CREB-binding protein (CBP)/p300, WD repeat-containing protein 5 (WDR5), DDB1- and CUL4-associated factor 1 (DCAF1), phosphatidylinositol-3 kinase (PI3K), or WEE1. In some embodiments, DDP is Aurora Kinase A (AurkA), Checkpoint kinase 1 (CHK1), CHK2, CDK4, CDK6, Myc, SMARCA2, SMARCA4, CREB-binding protein (CBP)/p300, WD repeat-containing protein 5 (WDR5), DDB1- and CUL4-associated factor 1 (DCAF1), phosphatidylinositol-3 kinase (PI3K), or WEE1. In some embodiments, the DDP is CBP/p300 or SMARCA2/4. In some embodiments, DDP is CBP/p300. In some embodiments, the DDP is SMARCA 2/4.

In some embodiments, DDP is a human bromodomain-containing protein (BRD). In some embodiments, DDP is a human bromodomain-containing protein that is a Group Ia BRD, Group Ib BRD, Group II BRD, Group IIIa BRD, Group IIIb BRD, Group IIII BRD, Group IV BRD, Group V BRD, Group VI BRD, Group VI BRD, Group VIII BRD, or group IX BRD (e.g., see Zaware, N., Zhou, MM. Bromodomain biology and drug discovery. Nat Struct Mol Biol 26, 870-879 (2019)). In some embodiments, the Group Ia BRD is selected from PCAF, GCN5L2, p300/CBP, TAF1 and TAF1L. In some embodiments, the Group Ib BRD is selected from BRPFlA/B (BR140), BRPF2 (BRD1) BRPF3 and BRD8 (SMAP). In some embodiments, the Group II BRD is selected from histone methyltransferases ASH1L and MLL. In some embodiments, the Group IIIa BRD is selected from chromatin remodeling factors SMARCA2 (BRM), SMARCA4 (BRG1), BRD7, BRD9 and PBRM1 (polybromo). In some embodiments, the Group IIIb BRD is selected from ISWI family chromatin remodeling factors BAZ1A (ACF1), BAZ1B (WSTF, William-Beuren syndrome transcription factor), BAZ2A, BAZ2B, BPTF and CECR2. In some embodiments, the Group IV BRD is selected from ATPase family AAA domain-containing proteins ATAD2 and ATAD2B. In some embodiments, the Group V BRD is selected from BET family proteins BRD2, BRD3, BRD4 and testis-specific BRDT. In some embodiments, the Group VI BRD is selected from Tripartite-motif-containing (TRIM) family proteins TRIM24 (TIF1α), TRIM28 (TIF1β, KAP1), TRIM33 (TIF1γ) and TRIM66 (TIF1δ). In some embodiments, the Group VIII BRD is selected from speckled protein (SP) family proteins SP100, SP110, SP140 and SP140L. In some embodiments, the Group VIII BRD is selected from transcriptional corepressors ZMYND8 and ZMYDN11. In some embodiments, the Group IX is selected from WD-repeat proteins BRWD1 (WDR9), BRWD3 and PHIP (WDR11).

In some embodiments, DDP is a human bromodomain-containing protein (BRD) that is selected from PCAF, GCN5L2, p300/CBP, TAF1 and TAF1L. In some embodiments, DDP is a human bromodomain-containing protein (BRD) that is selected from BRPF1A/B (BR140), BRPF2 (BRD1) BRPF3 and BRD8 (SMAP). In some embodiments, DDP is a human bromodomain-containing protein (BRD) that is selected from histone methyltransferases ASH1L and MLL. In some embodiments, DDP is a human bromodomain-containing protein (BRD) that is selected from chromatin remodeling factors SMARCA2 (BRM), SMARCA4 (BRG1), BRD7, BRD9 and PBRM1 (polybromo). In some embodiments, DDP is a human bromodomain-containing protein (BRD) that is selected from ISWI family chromatin remodeling factors BAZ1A (ACF1), BAZ1B (WSTF, William-Beuren syndrome transcription factor), BAZ2A, BAZ2B, BPTF and CECR2. In some embodiments, DDP is a human bromodomain-containing protein (BRD) that is selected from ATPase family AAA domain-containing proteins ATAD2 and ATAD2B. In some embodiments, DDP is a human bromodomain-containing protein (BRD) that is selected from Tripartite-motif-containing (TRIM) family proteins TRIM24 (TIF1α), TRIM28 (TIF1β, KAP1), TRIM33 (TIF1γ) and TRIM66 (TIF1δ). In some embodiments, DDP is a human bromodomain-containing protein (BRD) that is selected from speckled protein (SP) family proteins SP100, SP110, SP140 and SP140L. In some embodiments, DDP is a human bromodomain-containing protein (BRD) that is selected from transcriptional corepressors ZMYND8 and ZMYDN11. In some embodiments, DDP is ahuman bromodomain-containing protein (BRD) that is selected from WD-repeat proteins BRWD1 (WDR9), BRWD3 and PHIP (WDR11).

In some embodiments, when the disease protein is the androgen receptor (AR), then the DDP is an AR coactivator or coregulator. Androgens, functioning through the AR, are essential for the normal development and maintenance of the prostate. Androgen-bound AR functions as a transcription factor to regulate genes involved in an array of physiological processes. The transcriptional activity of AR is affected by coregulators that influence a number of functional properties of AR, including ligand selectivity and DNA binding capacity. As the promoter of target genes, coregulators participate in DNA modification, either directly through modification of histones or indirectly by the recruitment of chromatin-modifying complexes, as well as functioning in the recruitment of the basal transcriptional machinery. Aberrant coregulator activity due to mutation or altered expression levels may be a contributing factor in the progression of diseases related to AR activity, such as prostate cancer.

The progression of prostate cancer is also sensitive to androgens. Surgical and/or pharmacological androgen ablation remain the predominant form of treatment for advanced prostate cancer. Androgen ablation therapy is often combined with treatment with nonsteroidal antiandrogens, such as hydroxyflutamide, to block residual adrenal androgen action. While 70-80% of patients initially respond to androgen ablation therapy, tumors ultimately become resistant and may, in fact, proliferate in response to antiandrogens. Because AR is generally expressed in prostate tumors and their metastases, aberrant regulation of AR activity by coregulators may contribute to prostate cancer progression or the acquired agonist effect of antiandrogens.

In addition, androgen-independent activation of the AR is a well-known phenomenon and can occur via several different mechanisms, including activation by interleukins. For example, interleukin-6 (IL-6) has been shown to activate AR-dependent gene expression in the absence of androgens. Activation of the AR and AR target gene expression by IL-6 requires p300 and its HAT activity. Similar to IL-6, interleukin-4 (IL-4) activates the AR, increases CBP/p300 protein expression, and enhances the interaction of CBP/p300 with the AR at the KLK3 promoter. Therefore, CBP/p300 appears to be crucial for AR transcriptional activity in both the presence and absence of androgens.

In some embodiments, AR coactivators or coactivator or coregulator is AKT1, AurkA, BAG family molecular chaperone regulator 1 (BAG1), beta-catenin, Breast cancer type 1 susceptibility protein (BRCA1), BRD4, C-jun, calmodulin 1, caveolin 1, CDK4/6, CDK9, Cytochrome C oxidase subunit 5b (COX5B), CBP/p300, CD1, CDK7, Dachshund family transcription factor 1 (DACH1), Death domain associated protein (Daxx), DCAF1, L-3,4-dihydroxyphenylalanine (L-DOPA), EF-hand calcium-binding domain-containing protein 6 (EFCAB6), Epidermal growth factor receptor (EGFR), Forkhead Box 01 (FOXO1), Glyceraldehyde-3-Phosphate Dehydrogenase (GAPDH), gelsolin, guanine nucleotide-binding protein subunit beta-2-like 1 (GNB2L1), Glycogen synthase kinase 3 beta (GSK3B), HDAC1, Heat shock protein 90 alpha family class a member 1 (HSP90AA1), HTATIP, MAGEAI1, MED1, Myc, MYST2, Nuclear receptor coactivator 1 (NCOA1), NCOA2, NCOA3, NCOA4, NCOA6, Nuclear receptor corepressor 2 (NCOR2), Non-POU domain-containing octamer-binding protein (NONO), Proliferation-associated protein 2G4 (PA2G4), P21 (RAC1) Activated kinase 6 (PAK6), POZ/BTB and at hook containing zinc finger 1 (PATZ1), Protein Inhibitor Of Activated STAT 2 (PIAS2), Pre-MRNA Processing Factor 6 (PRPF6), Phosphatase And Tensin Homolog (PTEN), RAD9A, RAN binding protein 9 (RANBP9), Ring finger and CHY zinc finger domain containing 1 (RCHY1), Retinoblastoma protein, Ring finger protein 4 (RNF4), RNF14, Spliceosome associated factor 3, U4/U6 recycling protein (SART3), Sirtuin 1 (SIRTI), SMAD3, SMARCA2/4, small heterodimer partner, Src, Sex determining region Y (SRY), STAT3, Supervillin (SVIL), Testicular receptor 2, Testicular receptor 4, Transforming growth factor beta 1 induced transcript 1 (TGFB1I1), TATA element modulatory factor 1 (TMF1), Tripartite motif containing 68 (TRIM68), Ubiquitin conjugating enzyme E2 I (UBE2I), Ubiquitously expressed prefoldin like chaperone (UXT), WEE1, or Zinc finger MIZ-type containing 1 (ZMIZ1).

### Silent Binder of a Disease-Dependent Protein (SBDDP)

“Silent-binder” refers to a compound (or fragment of a heterobifunctional compound) that binds to a target protein and does not substantially alter protein function. In some embodiments, a silent binder does not result in functional activation or inhibition of the target protein.

It is understood that some silent binders may, upon binding to a target protein, result in or induce some measurable or detectable effect on protein function. However, in such instances the measurable altered protein function is not detrimental to the ordinary functioning of the target protein at concentrations relevant for inducing inhibition of the target protein.

It is also understood that the silent binder to a target protein does not substantially alter protein function when it is incorporated into the heterobifunctional conditional inhibitor compounds disclosed herein unless the non-silent binder (NSB) component of the heterobifunctional conditional inhibitor compound also simultaneously binds to the disease protein (DP) in the same cell.

In some embodiments, a silent binder may be silent because it binds to a domain of a target protein that is not a relevant domain for the activity of the target protein. In other embodiments, a silent binder may be silent because it an allosteric binder of the target protein. In other embodiments, a silent binder may be silent because its incorporation into a heterobifunctional compound reduces the activity of the binder as compared to when it is not incorporated into a heterobifunctional compound. For example, a compound may be a modulator of a target protein when it is not a component of a heterobifunctional compound but becomes a silent binder of the target protein by virtue of its incorporation into a heterobifunctional compounds disclosed herein.

In some embodiments, a silent binder may be silent because it binds to the bromodomain of a human bromodomain-containing protein (BRD). In some embodiments, a silent binder binds in the acetyl-lysine (KAc) binding site of the human bromodomain-containing protein (BRD). In some embodiments, a silent binder comprises a acetyl-lysine (KAc) mimetic moiety and binds in the acetyl-lysine (KAc) binding site of the human bromodomain-containing protein (BRD). For a list of human bromodomain-containing proteins and representative binders to such proteins see, eg., Czerwinska P, Mackiewicz A A. Bromodomain (BrD) Family Members as Regulators of Cancer Stemness-A Comprehensive Review. Int J Mol Sci. 2023 Jan. 4; 24(2):995; Fujisawa T, Filippakopoulos P. Functions of bromodomain-containing proteins and their roles in homeostasis and cancer. Nat Rev Mol Cell Biol. 2017 April; 18(4):246-262; Zaware, N., Zhou, M M. Bromodomain biology and drug discovery. Nat Struct Mol Biol 26, 870-879 (2019); Filippakopoulos, P., Knapp, S. Targeting bromodomains: epigenetic readers of lysine acetylation. Nat Rev Drug Discov 13, 337-356 (2014); each of which is herein incorporated by reference for such proteins and the representative ligands that bind to such proteins. In some embodiments, a silent binder binds to the bromodomain of a human bromodomain-containing protein (BRD). In some embodiments, DDP is a Group Ia BRD, Group Ib BRD, Group II BRD, Group IIIa BRD, Group IIIb BRD, Group IIII BRD, Group IV BRD, Group V BRD, Group VI BRD, Group VI BRD, Group VIII BRD, or group IX BRD (e.g., see Zaware, N., Zhou, M M. Bromodomain biology and drug discovery. Nat Struct Mol Biol 26, 870-879 (2019)) and the SBDDP binds to the bromodomain of the DDP. In some embodiments, DDP is PCAF, GCN5L2, p300/CBP, TAF1 or TAF1L and the SBDDP binds to the bromodomain of the DDP. In some embodiments, DDP is BRPF1A/B (BR140), BRPF2 (BRD1) BRPF3 or BRD8 (SMAP) and the SBDDP binds to the bromodomain of the DDP. In some embodiments, DDP is ASH1L or MLL and the SBDDP binds to the bromodomain of the DDP. In some embodiments, DDP is SMARCA2 (BRM), SMARCA4 (BRG1), BRD7, BRD9 or PBRM1 (polybromo) and the SBDDP binds to the bromodomain of the DDP. In some embodiments, DDP is BAZ1A (ACF1), BAZ1B (WSTF, William-Beuren syndrome transcription factor), BAZ2A, BAZ2B, BPTF or CECR2 and the SBDDP binds to the bromodomain of the DDP. In some embodiments, DDP is ATAD2 or ATAD2B and the SBDDP binds to the bromodomain of the DDP. In some embodiments, DDP is BRD2, BRD3, BRD4 or testis-specific BRDT and the SBDDP binds to the bromodomain of the DDP. In some embodiments, DDP is TRIM24 (TIF1α), TRIM28 (TIF1β, KAP1), TRIM33 (TIF1γ) or TRIM66 (TIF1δ) and the SBDDP binds to the bromodomain of the DDP. In some embodiments, DDP is SP100, SP110, SP140 or SP140L and the SBDDP binds to the bromodomain of the DDP. In some embodiments, DDP is ZMYND8 or ZMYDN11 and the SBDDP binds to the bromodomain the DDP. In some embodiments, DDP is BRWD1 (WDR9), BRWD3 or PHIP (WDR11) and the SBDDP binds to the bromodomain of the DDP.

In some embodiments, DDP is PCAF and the SBDDP binds to the bromodomain of PCAF. In some embodiments, DDP is GCN5L2 and the SBDDP binds to the bromodomain of GCN5L2. In some embodiments, DDP is p300/CBP and the SBDDP binds to the bromodomain of p300/CBP. In some embodiments, DDP is TAF1 and the SBDDP binds to the bromodomain of TAF1. In some embodiments, DDP is TAF1L and the SBDDP binds to the bromodomain of TAF1L.

In some embodiments, DDP is BRPF1A/B (BR140) and the SBDDP binds to the bromodomain of BR140. In some embodiments, DDP is BRPF2 (BRD1) and the SBDDP binds to the bromodomain of BRD1. In some embodiments, DDP is BRPF3 and the SBDDP binds to the bromodomain of BRPF3. In some embodiments, DDP is BRD8 (SMAP) and the SBDDP binds to the bromodomain of SMAP.

In some embodiments, DDP is ASH1L and the SBDDP binds to the bromodomain of ASH1L. In some embodiments, DDP is MLL and the SBDDP binds to the bromodomain of MLL.

In some embodiments, DDP is SMARCA2 (BRM) and the SBDDP binds to the bromodomain of SMARCA2. In some embodiments, DDP is SMARCA4 (BRG1) and the SBDDP binds to the bromodomain of BRG1. In some embodiments, DDP is BRD7 and the SBDDP binds to the bromodomain of BRD7. In some embodiments, DDP is BRD9 and the SBDDP binds to the bromodomain of BRD9. In some embodiments, DDP is PBRM1 (polybromo) and the SBDDP binds to the bromodomain of PBRM1. In some embodiments, DDP is BAZ1A (ACF1) and the SBDDP binds to the bromodomain of BAZ1A. In some embodiments, DDP is BAZ1B (WSTF, William-Beuren syndrome transcription factor) and the SBDDP binds to the bromodomain of BAZ1B. In some embodiments, DDP is BAZ2A and the SBDDP binds to the bromodomain of BAZ2A. In some embodiments, DDP is BAZ2B and the SBDDP binds to the bromodomain of BAZ2B. In some embodiments, DDP is BPTF and the SBDDP binds to the bromodomain of BPTF. In some embodiments, DDP is CECR2 and the SBDDP binds to the bromodomain of CECR2.

In some embodiments, DDP is ATAD2 and the SBDDP binds to the bromodomain ATAD2 of. In some embodiments, DDP is ATAD2B and the SBDDP binds to the bromodomain of ATAD2B.

In some embodiments, DDP is TRIM24 (TIF1α) and the SBDDP binds to the bromodomain of TIF1α. In some embodiments, DDP is TRIM28 (TIF1β, KAP1) and the SBDDP binds to the bromodomain of TRIM28. In some embodiments, DDP is TRIM33 (TIF1γ) and the SBDDP binds to the bromodomain of TIF1γ. In some embodiments, DDP is TRIM6δ (TIF1δ) and the SBDDP binds to the bromodomain of TIF1δ. In some embodiments, DDP is SP100 and the SBDDP binds to the bromodomain of SP100. In some embodiments, DDP is SP110 and the SBDDP binds to the bromodomain of SP110. In some embodiments, DDP is SP140 and the SBDDP binds to the bromodomain of SP140. In some embodiments, DDP is SP140L and the SBDDP binds to the bromodomain of SP140L.

In some embodiments, DDP is ZMYND8 and the SBDDP binds to the bromodomain of ZMYND8. In some embodiments, DDP is ZMYDN11 and the SBDDP binds to the bromodomain of ZMYDN11.

In some embodiments, DDP is BRWD1 (WDR9) and the SBDDP binds to the bromodomain of WDR9. In some embodiments, DDP is BRWD3 and the SBDDP binds to the bromodomain of BRWD3. In some embodiments, DDP is PHIP (WDR11) and the SBDDP binds to the bromodomain of WDR11.

“Binder of a disease protein” or “BDP” refers to a compound (or fragment of a heterobifunctional compound) that binds to a target disease protein and: 1) does not substantially alter protein function; or 2) substantially alters protein function. In some embodiments, a binder of a disease protein when incorporated into the heterobifunctional compounds disclosed herein does not substantially alter protein function of the disease protein. In some embodiments, a binder of a disease protein when incorporated into the heterobifunctional compounds disclosed herein does not substantially alter protein function of the disease protein unless the silent binder component of the heterobifunctional conditional compound also simultaneously binds to a disease-dependent protein in the same cell.

“Non-silent binder” or “NSB” refers to a compound (or fragment of a heterobifunctional compound) that binds to a target disease protein and substantially alters protein function. NSBs include, but are not limited to, agonist, inverse agonist, antagonist, neutral antagonist, silent antagonist, competitive antagonist, irreversible antagonist, reversible antagonist, inhibitor, irreversible inhibitor, reversible inhibitor, allosteric modulator, negative allosteric modulator, and positive allosteric modulator.

Silent Antagonist is a drug that attenuates the effects of agonists or inverse agonists, producing a functional reduction in signal transduction. Affects only ligand-dependent receptor activation and displays no intrinsic activity itself. Also known as a neutral antagonist.

Agonist is a drug that binds to and activates a receptor. Can be full, partial or inverse. A full agonist has high efficacy, producing a full response while occupying a relatively low proportion of receptors. A partial agonist has lower efficacy than a full agonist. It produces sub-maximal activation even when occupying the total receptor population, therefore cannot produce the maximal response, irrespective of the concentration applied. An inverse agonist produces an effect opposite to that of an agonist yet binds to the same receptor binding-site as an agonist.

Allosteric Modulator is a drug that binds to a receptor at a site distinct from the active site. Induces a conformational change in the receptor, which alters the affinity of the receptor for the endogenous ligand. Positive allosteric modulators increase the affinity, whilst negative allosteric modulators decrease the affinity.

Antagonist is a drug that attenuates the effect of an agonist. Can be competitive or non-competitive, each of which can be reversible or irreversible. A competitive antagonist binds to the same site as the agonist but does not activate it, thus blocks the agonist's action. A non-competitive antagonist binds to an allosteric (non-agonist) site on the receptor to prevent activation of the receptor. A reversible antagonist binds non-covalently to the receptor, therefore can be “washed out”. An irreversible antagonist binds covalently to the receptor and cannot be displaced by either competing ligands or washing.

Efficacy describes the way that agonists vary in the response they produce when they occupy the same number of receptors. High efficacy agonists produce their maximal response while occupying a relatively low proportion of the total receptor population. Lower efficacy agonists do not activate receptors to the same degree and may not be able to produce the maximal response (see Agonist, Partial).

In some embodiments, SBDDP is a CBP/p300 binder. In some embodiments, SBDDP is a CBP/p300 bromodomain binder. In some embodiments, SBDDP is a CBP/p300 histone acetyltransferase (HAT) domain binder. In some embodiments, SBDDP is a CBP/p300 bromodomain binder comprising a benzimidazole, piperidine, benzodiazepinone, indole, oxazolidinedione, barbituric skeleton, thiobarbituric skeleton, or alkaloid. In some embodiments, SBDDP is a CBP/p300 binder as described in Zhang-Xu He, et al., European Journal of Medicinal Chemistry, Volume 209, 2021, 112861, which is incorporated by reference for such CBP/p300 binder. In some embodiments, SBDDP is a CBP/p300 binder as any one of compounds 1 to 75 as described in Zhang-Xu He, et al., European Journal of Medicinal Chemistry, Volume 209, 2021, 112861, which is incorporated by reference for such CBP/p300 binders.

In some embodiments, SBDDP has one of the following structures:

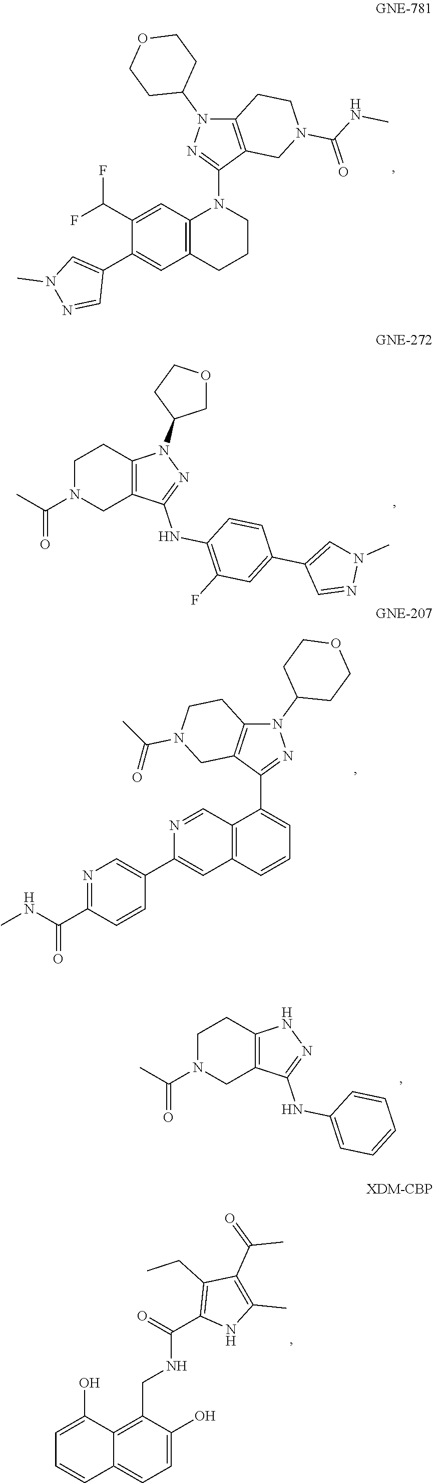

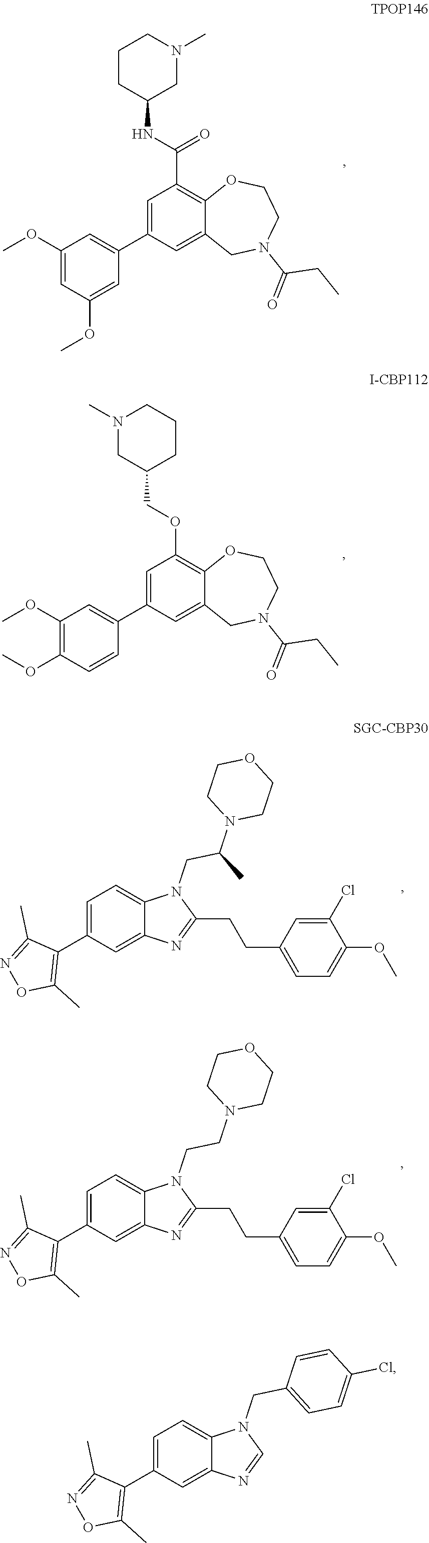

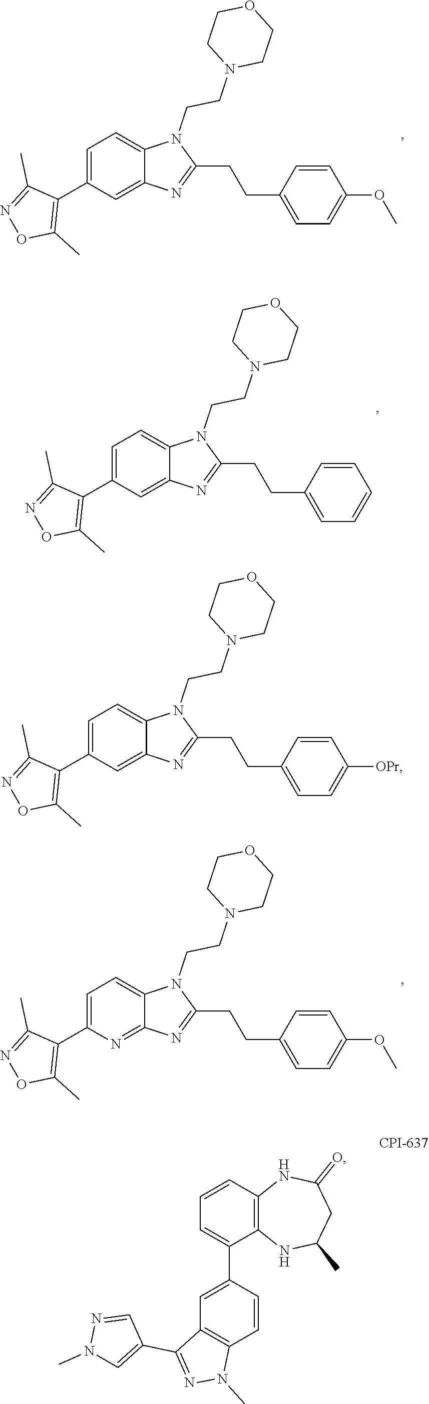

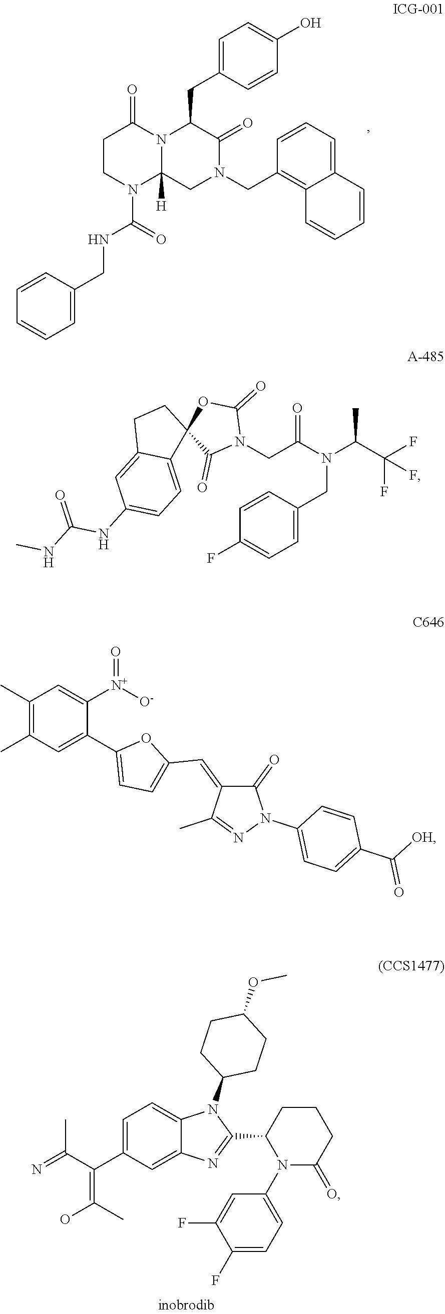

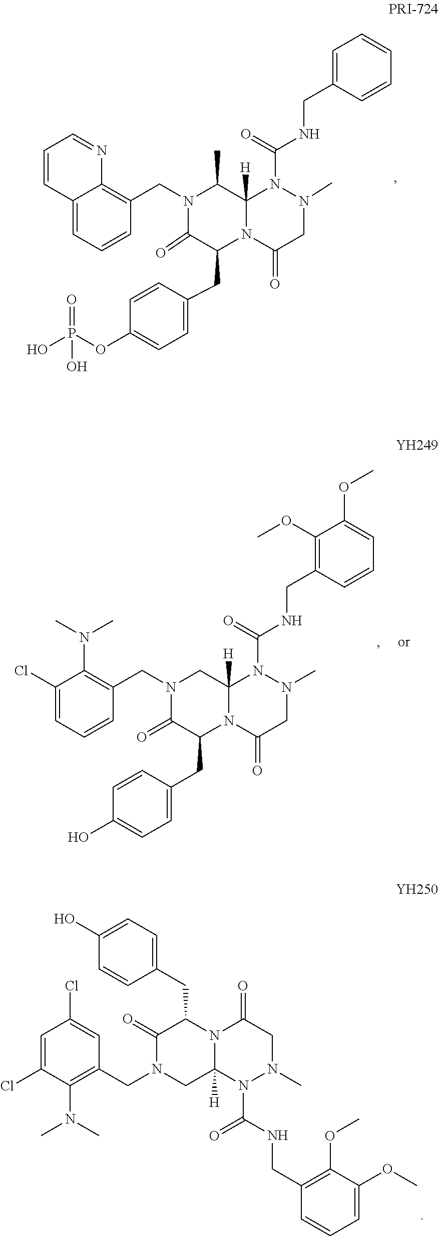 [SMILES: C[C@H]1[C@H]2N(C(=O)CN(C)N2C(=O)NCc2ccccc2)[C@@H](Cc2ccc(OP(=O)(O)O)cc2)C(=O)N1Cc1cccc2cccnc12 ; COc1cccc(CNC(=O)N2[C@H]3CN(Cc4cccc(Cl)c4N(C)C)C(=O)[C@H](Cc4ccc(O)cc4)N3C(=O)CN2C)c1OC ; COc1cccc(CNC(=O)N2[C@H]3CN(Cc4cc(Cl)cc(Cl)c4N(C)C)C(=O)[C@H](Cc4ccc(O)cc4)N3C(=O)CN2C)c1OC]

In some embodiments, SBDDP has one of the following structures:

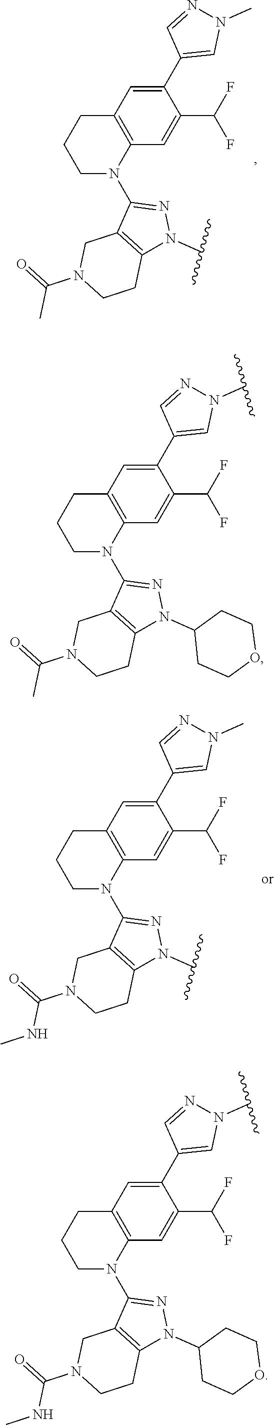

### Disease Protein (DP)

In some embodiments, the DP is a protein selectively expressed in a cancer or COI.

In some embodiments, the DP is a nuclear hormone receptor protein. In some embodiments, the DP is a nuclear hormone receptor, for example, androgen receptor (AR), estrogen receptor (ER), retinoic acid receptor (RAR), vitamin D receptor (VDR), glucocorticoid receptor (GR), mineralocorticoid receptor (MR), or progesterone receptor (PR). In some embodiments, the DP is AR or ER. In some embodiments, the DP is AR.

In some embodiments, the DP is a membrane receptor. In some embodiments, the DP is an ion channel linked receptor, an enzyme linked receptor, or a G-protein coupled receptor.

In some embodiments, the DP is a G-protein coupled receptor (GPCR). In some embodiments, the DP is angiotensin receptor, apelin receptor, bombesin receptor, bradykinin receptor, calcitonin receptor, chemokine receptor, cholecytokinin receptor, corticotropic-releasing factor receptor, galanin receptor, ghrelin receptor, glucagon receptor, glycoprotein hormone receptor, gonadotropin-releasing hormone receptor, kisspeptin receptor, melanocortin receptor, motilin receptor, neuromedin U receptor, neuropeptide FF/AF receptor, neuropeptide S receptor, neuropeptide W/B receptor, neuropeptide Y receptor, opioid receptor, orexin receptor, parathyroid hormone receptor, prokineticin receptor, prolactin-releasing peptide receptor, QRFP receptor, relaxin family peptide receptor, somatostatin receptor, tachykinin receptor, thyrotropin-releasing hormone receptor, urotensin receptor, vasopressin, oxytocin receptor, vasoactive intestinal peptide receptor, or PACAP receptor.

In some embodiments, the DP is an enzyme linked receptor. In some embodiments, the DP is a receptor tyrosine kinase, serine/threonine kinase, or a tyrosine kinase-associated receptor. In some embodiments, the DP is a receptor tyrosine kinase, for example, an epidermal growth factor receptor, nerve growth factor receptor, insulin receptor, or toll like receptor. In some embodiments, the DP is an epidermal growth factor receptor, for example, HER1, HER2, HER3, or HER4. In some embodiments, the DP is HER2.

In some embodiments, the DP is a GTPase. In some embodiments, the DP is a Ras protein. In some embodiments, the DP is a KRAS, HRAS, or NRAS protein. In some embodiments, the DP is a KRAS protein. In some embodiments, the DP is KRAS-G12C.

In some embodiments, the DP is a transcription factor. In some embodiments, the DP is B-Cell Lymphoma 6 (BCL6).

In some embodiments, the DP is AR, BCL6, ER, HER2, or KRAS-G12C. In some embodiments, the DP is AR or ER. In some embodiments, the DP is AR. In some embodiments, the DP is BCL6.

In some embodiments, the DP is a nuclear hormone receptor protein, G-protein coupled receptor, epidermal growth factor receptor, RAS protein, or transcription factor. In some embodiments, the DP is DP is androgen receptor (AR), estrogen receptor (ER), progesterone receptor (PR), glucocorticoid receptor (GR), mineralocorticoid receptor (MR), angiotensin receptor, apelin receptor, bombesin receptor, bradykinin receptor, calcitonin receptor, chemokine receptor, cholecytokinin receptor, corticotropic-releasing factor receptor, galanin receptor, ghrelin receptor, glucagon receptor, glycoprotein hormone receptor, gonadotropin-releasing hormone receptor, kisspeptin receptor, melanocortin receptor, motilin receptor, neuromedin U receptor, neuropeptide FF/AF receptor, neuropeptide S receptor, neuropeptide W/B receptor, neuropeptide Y receptor, opioid receptor, orexin receptor, parathyroid hormone receptor, prokineticin receptor, prolactin-releasing peptide receptor, QRFP receptor, relaxin family peptide receptor, somatostatin receptor, tachykinin receptor, thyrotropin-releasing hormone receptor, urotensin receptor, vasopressin, oxytocin receptor, vasoactive intestinal peptide receptor, PACAP receptor, HER1, HER2, HER3, HER4, KRAS, KRAS-G12C, HRAS, NRAS, or BCL6. In some embodiments, the DP is AR or ER. In some embodiments, the DP is AR.

In some embodiments, the DP is a transcription factor (TF) selected from the nuclear receptor (NR) superfamily (e.g., see Weikum E R, Liu X, Ortlund E A. The nuclear receptor superfamily: A structural perspective. Protein Sci. 2018 November; 27(11):1876-1892). In some embodiments, NR ligand binding domain

In some embodiments, the DP is androgen receptor (AR), estrogen receptor (ER), progesterone receptor (PR), glucocorticoid receptor (GR), mineralocorticoid receptor (MR), retinoic acid receptor-related orphan nuclear receptor γ (RORγ), angiotensin receptor, apelin receptor, bombesin receptor, bradykinin receptor, calcitonin receptor, chemokine receptor, cholecytokinin receptor, chymase, corticotropic-releasing factor receptor, galanin receptor, ghrelin receptor, glucagon receptor, glycoprotein hormone receptor, gonadotropin-releasing hormone receptor, doleamine 2,3-dioxygenase 1 (IDO1), kisspeptin receptor, melanocortin receptor, motilin receptor, neuromedin U receptor, neuropeptide FF/AF receptor, neuropeptide S receptor, neuropeptide W/B receptor, neuropeptide Y receptor, opioid receptor, orexin receptor, parathyroid hormone receptor, prokineticin receptor, prolactin-releasing peptide receptor, QRFP receptor, relaxin family peptide receptor, somatostatin receptor, tachykinin receptor, thyrotropin-releasing hormone receptor, urotensin receptor, vasopressin, oxytocin receptor, vasoactive intestinal peptide receptor, PACAP receptor, HER1, HER2, HER3, HER4, KRAS, KRAS-G12C, HRAS, NRAS, or BCL6.

### Binder of a Disease Protein (BDP)

In some embodiments, the BDP is a non-silent binder of a DP. In some embodiments, the BDP is a silent binder of a DP.

In some embodiments, the DP is a transcription factor (TF) selected from the nuclear receptor (NR) superfamily and BDP binds to the ligand binding domain of the DP. In some embodiments, the DP is thyroid hormone receptor (TR) and BDP binds to the ligand binding domain of the DP. In some embodiments, the DP is a retinoic acid receptor (RAR) and BDP binds to the ligand binding domain of the DP. In some embodiments, the DP is a peroxisome proliferator activated receptor (PPAR) and BDP binds to the ligand binding domain of the DP. In some embodiments, the DP is retinoic acid related receptors (ROR) and BDP binds to the ligand binding domain of the DP. In some embodiments, the DP is farnesoid X receptor (FXR) and BDP binds to the ligand binding domain of the DP. In some embodiments, the DP is liver X receptor (LXR) and BDP binds to the ligand binding domain of the DP. In some embodiments, the DP is retinoid X receptor (RXR) and BDP binds to the ligand binding domain of the DP. In some embodiments, the DP is androgen receptor (AR) and BDP binds to the ligand binding domain of the DP. In some embodiments, the DP is progesterone receptor (PR) and BDP binds to the ligand binding domain of the DP. In some embodiments, the DP is glucocorticoid receptor (GR) and BDP binds to the ligand binding domain of the DP. In some embodiments, the DP is mineralocorticoid receptor (MR) and BDP binds to the ligand binding domain of the DP.

In some embodiments, the DP is androgen receptor (AR) and the BDP is an AR antagonist. In some embodiments, the BDP is an AR antagonist that is flutamide, hydroxylflutamide, nilutamide, bicalutamide, enzalutamide, apalutamide, ODM201, AZD3514, or BMS641988.

### Representative Approaches to Designing Heterobifunctional Compounds

The heterobifunctional compounds described herein include three component parts: the ligand that binds the target protein (i.e., the disease protein), the ligand that binds to the disease-dependent protein (DDP), and the linker. Crystal structures of many of these proteins are known and the binding interactions of each component within its respective binding site can be visualized with suitable modeling software. In addition, computer modeling can also be performed to determine the placement of the two proteins relative to one another.

The Protein Data Bank (PDB) is a database for the three-dimensional structural data of large biological molecules, such as proteins and nucleic acids (Nucleic Acids Res. 2019 Jan. 8; 47(D1):D520-D528. doi: 10.1093/nar/gky949). The data is submitted by biologists and biochemists from around the world, are freely accessible on the Internet via the websites of its member organizations (e.g. PDBe—www.pdbe.org, PDBj—www.pdbj.org, RCSB—www.rcsb.org/pdb, and BMRB—www.bmrb.wisc.edu). The PDB is overseen by an organization called the Worldwide Protein Data Bank—wwPDB—www.wwpdb.org.

In some embodiments, searching on the PDB database and manipulating of protein 3D models is performed with PyMOL. PyMOL is a user-sponsored molecular visualization system on an open-source foundation, maintained and distributed by Schrödinger. Other PDB database search engines and molecular visualization system are contemplated.

Model of ligand docking in candidate proteins, and modelling of ternary complexes can be built in the Rosetta software suite (Leaver-Fay A, et al., ROSETTA3: an object-oriented software suite for the simulation and design of macromolecules. Methods Enzymol. 2011; 487:545-74). In addition, structural information of candidate proteins and binders to the candidate proteins exist, see e.g., Filippakopoulos, P., Knapp, S. Targeting bromodomains: epigenetic readers of lysine acetylation. Nat Rev Drug Discov 13, 337-356 (2014); Filippakopoulos, P. et al. Histone recognition and large-scale structural analysis of the human bromodomain family. Cell 149, 214-231 (2012); C. Ren, L. Zeng, M.-M. Zhou, Chapter Fourteen—Preparation, Biochemical Analysis, and Structure Determination of the Bromodomain, an Acetyl-Lysine Binding Domain, Editor(s): Ronen Marmorstein, Methods in Enzymology, Academic Press, Volume 573, 2016, Pages 321-343).

Novel silent binders can be identified for DDPs using methods known in the art, such as fragment-based lead discovery (FBLD) (Kirsch P.; et al., Concepts and Core Principles of Fragment-Based Drug Design. Molecules 2019, 24 (23), 4309; Lamoree B.; et al. Current perspectives in fragment-based lead discovery (FBLD). Essays Biochem. 2017, 61 (5), 453-464; Erlanson D. A.; et al. Fragment-Based Drug Discovery. J. Med. Chem. 2004, 47 (14), 3463-3482; Shuker S. B.; et al. Discovering high-affinity ligands for proteins: SAR by NMR. Science 1996, 274 (5292), 1531-1534), DNA-Encoded Library (DEL) approaches and traditional high-throughput screening (HTS) methods.

Screening libraries used in FBLD are composed of small molecules called fragments that are broadly compliant with what is now widely recognized as the rule-of-three (Ro3) (Congreve M.; et al., ‘Rule of Three’ for fragment-based lead discovery?. Drug Discovery Today 2003, 8 (19), 876-877; Jhoti H.; et al., The ‘rule of three’ for fragment-based drug discovery: where are we now?. Nat. Rev. Drug Discovery 2013, 12 (8), 644-644). The small size and simplicity of fragments enables a minimalist approach to drug discovery, whereby a vast chemical space can be efficiently covered using libraries of only a few hundred molecules. This makes fragment screening appealing to industry and academic investigators alike and represents a very accessible, cost-effective, and sustainable approach to hit discovery. Furthermore, unlike conventional libraries used for HTS, fragment libraries are not biased toward previously explored targets. This makes FBLD an indispensable method for assessing the ligandability of novel proteins' binding sites, including PPI sites.

In some embodiments, the small size of fragments, however, results in them having weak binding affinities to proteins. For this reason, fragments tend to be screened at high concentrations using biophysical techniques such as nuclear magnetic resonance (NMR), surface plasmon resonance (SPR), differential scanning fluorimetry (DSF; also known as thermal shift assay (TSA)), and X-ray crystallography. Weak binders can be skillfully optimized into bespoke potent ligands for proteins via fragment growing, merging, and hybridizing methodologies. However, the successful pursuit of such approaches is often contingent on the determination of the binding mode of the fragment hits to the target protein, typically using X-ray crystallographic methods and/or ¹⁵N 2D-NMR experiments and/or cryogenic electron microscopy (cryo-EM) (Saur M.; et al., Fragment-based drug discovery using cryo-EM. Drug Discovery Today 2020, 25 (3), 485-490).

DEL is achieved through combinatorial chemistry and DNA-encoding techniques. With library modularity, DELs can be built in a time-saving and labor-saving way. This technology can construct and screen unprecedented scale combinatorial compound libraries (hundreds of billions scale) and discover numerous high-affinity ligands with high efficiency and low cost through protein target affinity screening and high-throughput sequencing and decoding (Buller et al., (2010). Drug discovery with DNA-encoded chemical libraries. Bioconjug. Chem. 21 (9), 1571-15802010; Kalliokoski T. (2015). Price-focused analysis of commercially available building blocks for combinatorial library synthesis. ACS Comb. Sci. 17 (10), 600-607; Goodnow et al., (2017). DNA-Encoded chemistry: Enabling the deeper sampling of chemical space. Nat. Rev. Drug Discov. 16 (2), 131-147). DEL can be used to create compound libraries with higher molecular weight. Empirically, such DEL libraries appear well suited for discovering ligands for protein-protein interaction (PPI) targets, which are increasingly needed for hits.

Linkers can be designed and assessed as well. Capturing the breadth of viable linker conformations is much akin to modeling the flexibility of drug-like molecules when docking them to proteins. For this purpose, the OMEGA software (Hawkins P C, et al., Comparison of shape-matching and docking as virtual screening tools. J Med Chem. 2007; 50:74-82; Hawkins P C, Nicholls A. Conformer generation with OMEGA: learning from the data set and the analysis of failures. J Chem Inf Model. 2012; 52:2919-36).

### Compounds

In some embodiments, the heterobifunctional conditional inhibitor compound is a compound of Formula (I):

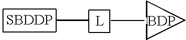

wherein:
SB is a silent binder of a disease-dependent protein (DDP);
L is an optional linker; and
BDP is a binder of a disease protein (DP).

In one aspect, described herein is a heterobifunctional conditional inhibitor compound of Formula (I):

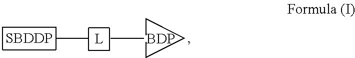

wherein:
SBDDP is a silent binder of a disease-dependent protein (DDP);
L is an optional linker; and
BDP is a binder of a disease protein (DP);
wherein DDP and DP are both expressed in a cell of interest (COI) and the relative abundance of the DP in the COI is greater than the relative abundance of the DDP in the COI; or
wherein the COI is a diseased cell, and the DP is overexpressed, overactive, or both overexpressed and overactive, or amplified in the diseased cell as compared to when the COI is a non-diseased cell.

In some embodiments, the activity of the DDP is reduced or inhibited by the compound of Formula (I) when the DDP and DP are both expressed in the same COI and the relative abundance of the DP in the COI is greater than the relative abundance of the DDP in the COI.

In some embodiments, DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3.

In some embodiments, DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

In some embodiments, DDP is a human bromodomain-containing protein (BRD).

In some embodiments, DDP is a human bromodomain-containing protein that is a Group Ia BRD, Group Ib BRD, Group II BRD, Group IIIa BRD, Group IIIb BRD, Group IIII BRD, Group IV BRD, Group V BRD, Group VI BRD, Group VI BRD, Group VIII BRD, or group IX BRD.

In some embodiments, the Group Ia BRD is selected from PCAF, GCN5L2, p300/CBP, TAF1 and TAF1L; the Group Ib BRD is selected from BRPF1A/B (BR140), BRPF2 (BRD1) BRPF3 and BRD8 (SMAP); the Group II BRD is selected from histone methyltransferases ASH1L and MLL; the Group IIIa BRD is selected from chromatin remodeling factors SMARCA2 (BRM), SMARCA4 (BRG1), BRD7, BRD9 and PBRM1 (polybromo); the Group IIIb BRD is selected from ISWI family chromatin remodeling factors BAZ1A (ACF1), BAZ1B (WSTF, William-Beuren syndrome transcription factor), BAZ2A, BAZ2B, BPTF and CECR2; the Group IV BRD is selected from ATPase family AAA domain-containing proteins ATAD2 and ATAD2B; the Group V BRD is selected from BET family proteins BRD2, BRD3, BRD4 and testis-specific BRDT; the Group VI BRD is selected from Tripartite-motif-containing (TRIM) family proteins TRIM24 (TIF1α), TRIM28 (TIF1β, KAP1), TRIM33 (TIF1γ) and TRIM66 (TIF1δ); the Group VIII BRD is selected from speckled protein (SP) family proteins SP100, SP110, SP140 and SP140L; the Group VIII BRD is selected from transcriptional corepressors ZMYND8 and ZMYDN11; and the Group IX is selected from WD-repeat proteins BRWD1 (WDR9), BRWD3 and PHIP (WDR11).

In some embodiments, DDP is a human bromodomain-containing protein (BRD) is: a Group Ia BRD selected from PCAF, GCN5L2, p300/CBP, TAF1 and TAF1L; a Group Ib BRD selected from BRPF1A/B (BR140), BRPF2 (BRD1) BRPF3 and BRD8 (SMAP); a Group II BRD selected from histone methyltransferases ASH1L and MLL; a Group IIIa BRD selected from chromatin remodeling factors SMARCA2 (BRM), SMARCA4 (BRG1), BRD7, BRD9 and PBRM1 (polybromo); a Group IIIb BRD selected from ISWI family chromatin remodeling factors BAZ1A (ACF1), BAZ1B (WSTF, William-Beuren syndrome transcription factor), BAZ2A, BAZ2B, BPTF and CECR2; a Group IV BRD selected from ATPase family AAA domain-containing proteins ATAD2 and ATAD2B; a Group VI BRD selected from Tripartite-motif-containing (TRIM) family proteins TRIM24 (TIF1α), TRIM28 (TIF1β, KAP1), TRIM33 (TIF1γ) and TRIM66 (TIF1δ); a Group VIII BRD selected from speckled protein (SP) family proteins SP100, SP110, SP140 and SP140L; a Group VIII BRD selected from transcriptional corepressors ZMYND8 and ZMYDN11; or a Group IX selected from WD-repeat proteins BRWD1 (WDR9), BRWD3 and PHIP (WDR11).

In another aspect, described herein is a heterobifunctional compound of Formula (Ia), comprising:

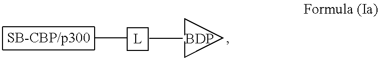

SB-CBP/p300 a silent binder of human CREB-binding protein (CBP) or human E1A-binding protein p300 (p300) (CBP/p300); BDP is a binder of a disease protein (DP); L is an optional linker covalently connecting SB-CBP/p300 to BDP; wherein L is covalently attached at a position of SB-CBP/p300 that is solvent exposed when SB-CBP/p300 binds to CBP/p300, and L is covalently attached at a position of BDP that is solvent exposed when BDP binds to DP; wherein CBP/p300 and DP are both expressed in a cell of interest (COI) and the relative abundance of the DP in the COI is greater than the relative abundance of CBP/p300 in the COI; or wherein the COI is a diseased cell, and the DP is overexpressed, overactive, or both overexpressed and overactive, or amplified in the diseased cell as compared to when the COI is a non-diseased cell.

In some embodiments, the DP is androgen receptor (AR), estrogen receptor (ER), progesterone receptor (PR), glucocorticoid receptor (GR), mineralocorticoid receptor (MR), retinoic acid receptor-related orphan nuclear receptor γ (RORγ), angiotensin receptor, apelin receptor, bombesin receptor, bradykinin receptor, calcitonin receptor, chemokine receptor, cholecytokinin receptor, chymase, corticotropic-releasing factor receptor, galanin receptor, ghrelin receptor, glucagon receptor, glycoprotein hormone receptor, gonadotropin-releasing hormone receptor, doleamine 2,3-dioxygenase 1 (IDO1), kisspeptin receptor, melanocortin receptor, motilin receptor, neuromedin U receptor, neuropeptide FF/AF receptor, neuropeptide S receptor, neuropeptide W/B receptor, neuropeptide Y receptor, opioid receptor, orexin receptor, parathyroid hormone receptor, prokineticin receptor, prolactin-releasing peptide receptor, QRFP receptor, relaxin family peptide receptor, somatostatin receptor, tachykinin receptor, thyrotropin-releasing hormone receptor, urotensin receptor, vasopressin, oxytocin receptor, vasoactive intestinal peptide receptor, PACAP receptor, HER1, HER2, HER3, HER4, KRAS, KRAS-G12C, HRAS, NRAS, or BCL6.

In some embodiments, SB-CBP/p300 binds to the bromodomain of human CREB-binding protein (CBP) or human ETA-binding protein p300 (p300) (CBP/p300).

In some embodiments, SB-CBP/p300 binds in the acetyl-lysine (KAc) binding site of the bromodomain of human CREB-binding protein (CBP) or human ETA-binding protein p300 (p300) (CBP/p300).

In some embodiments, the moiety comprising an acetyl-lysine mimetic makes or mimics a hydrogen bond interaction to Asn1168 in the Asn-binding pocket of the bromodomain of CBP, or makes or mimics a hydrogen bond interaction to Asn1132 in the Asn-binding pocket of the bromodomain of p300.

In some embodiments, SBDDP further comprises a moiety that interacts with Arg1173 in the bromodomain of CBP or Asn1137 in the bromodomain of p300.

In some embodiments, the moiety comprising an acetyl-lysine mimetic comprises a moiety selected from pyrrolidonyl, phenyl, pyridinyl, pyridinonyl, triazolyl, pyrrolyl, isoxazolyl, pyrazolyl, quinolinyl, tetrahydroquinolinyl, isoquinolinyl, tetrahydroisoquinolinyl, quinazolonyl, quinazolinyl, dihydroquinazolinonyl, imidazo[4,5-c]quinolinyl fused to a dimethylisoxazolyl, triazolophthalazinyl, indolizinyl, benzoimidazolyl, isoxazole-indolizinyl, thienodiazepine-indolizinyl, benzodiazepine-indolizinyl, 5-isoxazolylbenzimidazolyl, 6-isoxazolylbenzimidazolyl, 7-isoxazolo-quinolinyl, diazobenzyl, triazolophthalazinyl, isoxazoloquinolinyl, 2-thiazolidinonyl, triazolopyrimidinyl, thienodiazepinyl, benzodiazepinyl, benzotriazepinyl, triazolobenzodiazepinyl, triazolothienodiazepinyl, triazolothienodiazepinyl, and isoxazole-azepinyl.

In some embodiments, SB-CBP/p300 further comprises:
1) a moiety that occupies the lipophilic shelf (LPF) region of the bromodomain of CBP/p300; or
2) a moiety that occupies the BC Loop region of the bromodomain of CBP/p300; or
3) both 1) and 2);
wherein L is covalently attached to SB-CBP/p300 on:
   the acetyl-lysine mimetic moiety; or
   the moiety that occupies the lipophilic shelf (LPF) region of the bromodomain of CBP/p300, if present; or
   the moiety that occupies the BC Loop region of the bromodomain of CBP/p300, if present;
      wherein L is covalently attached to SB-CBP/p300 at a position that does not interfere with the binding of the acetyl-lysine mimetic moiety in the acetylated lysine (KAc) binding site of CBP/p300.

In some embodiments, the BDP is an AR antagonist that is flutamide, hydroxylflutamide, nilutamide, bicalutamide, enzalutamide, apalutamide, ODM201, AZD3514, or BMS641988.

In some embodiments disclosed herein are heterobifunctional conditional inhibitor compounds of Formula (III):

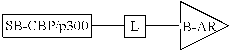

wherein:
SB-CBP/p300 is a silent binder of CBP/p300;
L is an optional linker; and
B-AR is a binder of the androgen receptor (AR).

In some embodiments, disclosed herein is a heterobifunctional compound of Formula (Ia), comprising:
a. a binder of human CREB-binding protein (CBP) or human E1A-binding protein p300 (p300) (CBP/p300);
b. a binder of a disease protein (DP); and
c. an optional linker covalently connecting a) to b);
   wherein the optional linker is covalently attached at a position of a) that is solvent exposed when a) binds to CBP/p300.

In some embodiments, the binder of CBP/p300 binds in the acetyl-lysine (Kac) binding site of the bromodomain CBP/p300. In some embodiments, the binder of CBP/p300 comprises an acetyl-lysine mimetic moiety that binds in the acetyl-lysine (KAc) binding site of the bromodomain of human CREB-binding protein (CBP) or human E1A-binding protein p300 (p300) (CBP/p300).

In some embodiments, the optional linker is covalently attached at a position of a) that is solvent exposed when a) binds the KAc binding site of the bromodomain of CBP/p300.

In some embodiments, the moiety comprising an acetyl-lysine mimetic makes or mimics a hydrogen bond interaction to Asn1168 in the Asn-binding pocket of the bromodomain of CBP, or makes or mimics a hydrogen bond interaction to Asn1132 in the Asn-binding pocket of the bromodomain of p300.

In some embodiments, a) further comprises a moiety that interacts with Arg1173 in the bromodomain of CBP or Asn1137 in the bromodomain of p300.

In some embodiments, wherein a) further comprises:
1) a moiety that occupies the lipophilic shelf (LPF) region of the bromodomain of CBP/p300; or
2) a moiety that occupies the BC Loop region of the bromodomain of CBP/p300; or
3) both 1) and 2).

In some embodiments, wherein the moiety comprising an acetyl-lysine mimetic comprises a moiety selected from pyrrolidonyl, phenyl, pyridinyl, pyridinonyl, triazolyl, pyrrolyl, isoxazolyl, pyrazolyl, quinolinyl, tetrahydroquinolinyl, isoquinolinyl, tetrahydroisoquinolinyl, quinazolonyl, quinazolinyl, dihydroquinazolinonyl, imidazo[4,5-c]quinolinyl fused to a dimethylisoxazolyl, triazolophthalazinyl, indolizinyl, benzoimidazolyl, isoxazole-indolizinyl, thienodiazepine-indolizinyl, benzodiazepine-indolizinyl, 5-isoxazolylbenzimidazolyl, 6-isoxazolylbenzimidazolyl, 7-isoxazolo-quinolinyl, diazobenzyl, triazolophthalazinyl, isoxazoloquinolinyl, 2-thiazolidinonyl, triazolopyrimidinyl, thienodiazepinyl, benzodiazepinyl, benzotriazepinyl, triazolobenzodiazepinyl, triazolothienodiazepinyl, triazolothienodiazepinyl, and isoxazole-azepinyl.

In some embodiments, the optional linker of c) is covalently attached to a) on: the acetyl-lysine mimetic moiety; or the moiety that occupies the lipophilic shelf (LPF) region of the bromodomain of CBP/p300, if present; or the moiety that occupies the BC Loop region of the bromodomain of CBP/p300, if present; wherein the optional linker of c) is covalently attached to a) at a position that does not interfere with the binding of the acetyl-lysine mimetic moiety in the acetylated lysine (KAc) binding site of CBP/p300.

In some embodiments, the moiety comprising an acetyl-lysine mimetic comprises a moiety selected from:

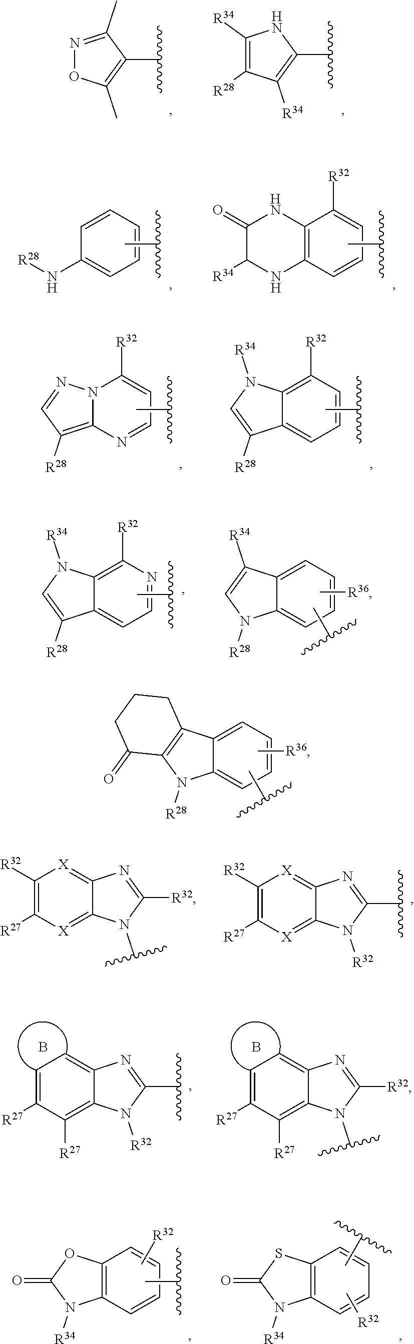

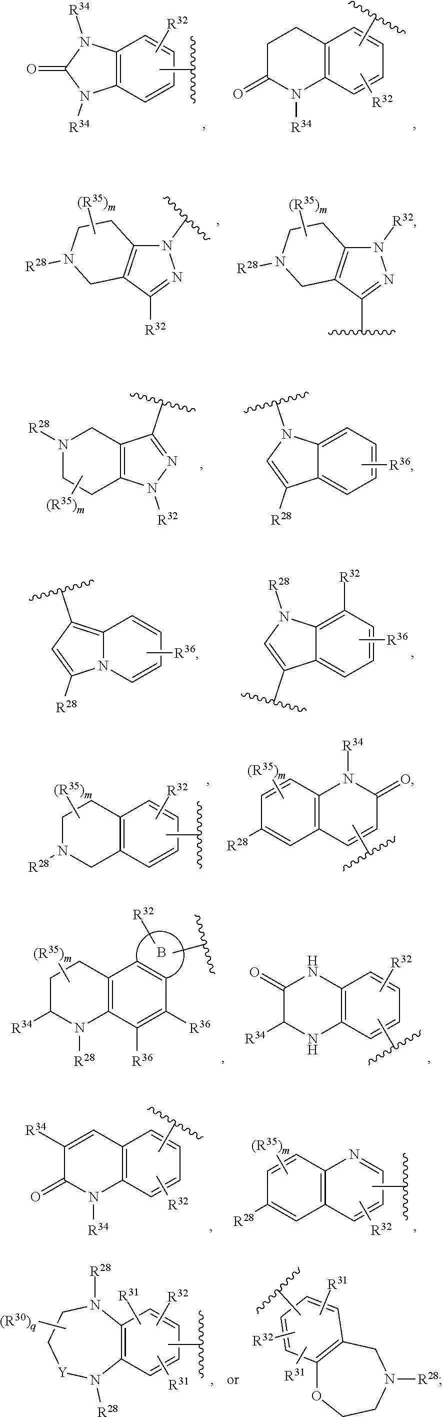

each R³² is independently an optional moiety that occupies the lipophilic shelf (LPF) region of the bromodomain of CBP/p300; or each R³² is independently an optional moiety that occupies the BC Loop region of the bromodomain of CBP/p300;
[symbol] is the point of attachment to the optional linker that covalently connects a) to b);
or [symbol] is R³² and the optional linker that covalently connects a) to b) is attached to R³²;
each R²⁸ is independently hydrogen, substituted or unsubstituted C₁-C₆alkyl, substituted or unsubstituted C₁-C₆fluoroalkyl, —C(═O)R^{b}, or —C(═O)N(R^{b})₂;
each R³⁴ is independently hydrogen or substituted or unsubstituted C₁-C₆alkyl;
each R³⁵ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl, —CN, —OH, —OR^{a}, or —N(R^{b})₂;
m is 0, 1, 2, 3, or 4;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
Y is —C(R³⁰)₂— or C(═O);
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
each R³¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)NR^{b}, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
each R³⁶ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR^{a}, or —N(R^{b})₂;
Ring B is a fused substituted or unsubstituted 5 or 6 membered heterocycloalkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl; and
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, the moiety that occupies the lipophilic shelf (LPF) region of the bromodomain of CBP/p300 is R³², wherein:
R³² is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₂cycloalkyl, substituted or unsubstituted 3- to 12-membered heterocycloalkyl, substituted or unsubstituted aryl, or substituted or unsubstituted heteroaryl;
or R³² is

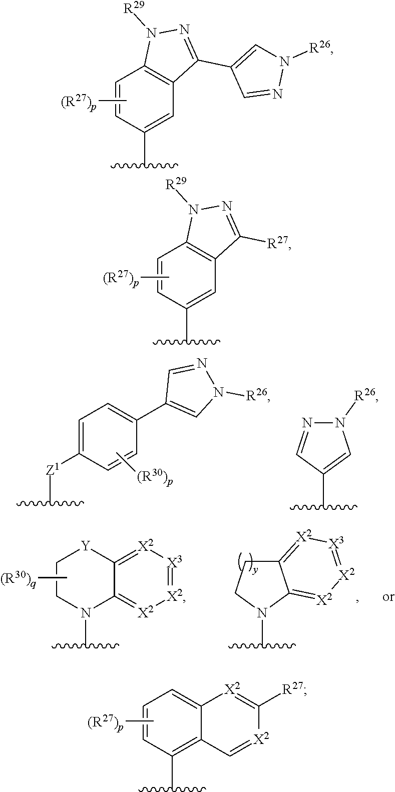

y is 1 or 2; Z¹ is —NR^{c}—, —O—, or —S—;
R^{c} is hydrogen or substituted or unsubstituted C₁-C₆alkyl;
R²⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each X² is independently —CR³⁰— or —N—; each X³ is independently —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen or substituted or unsubstituted C₁-C₆alkyl;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
Y is —C(R³⁰)₂— or N(R²⁸)—;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂; and
q is 0, 1, 2, 3, or 4;
or R³² is -L-C;
   L is substituted or unsubstituted C₁-C₆alkyl or substituted or unsubstituted C₁-C₆heteroalkyl;
   C is substituted or unsubstituted C₃-C₈cycloalkyl, substituted or unsubstituted 3- to 8-membered heterocycloalkyl, substituted or unsubstituted aryl, or substituted or unsubstituted heteroaryl;
   each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
   each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
   or two R^{b} on the same N atom are taken together with the N atom to which they are attached to form a substituted or unsubstituted N-containing heterocycle.

In some embodiments, the moiety that occupies the lipophilic shelf (LPF) region of the bromodomain of CBP/p300 is R³², wherein:
R³² is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₂cycloalkyl, substituted or unsubstituted 3- to 12-membered heterocycloalkyl, substituted or unsubstituted aryl, or substituted or unsubstituted heteroaryl;
or R³² is

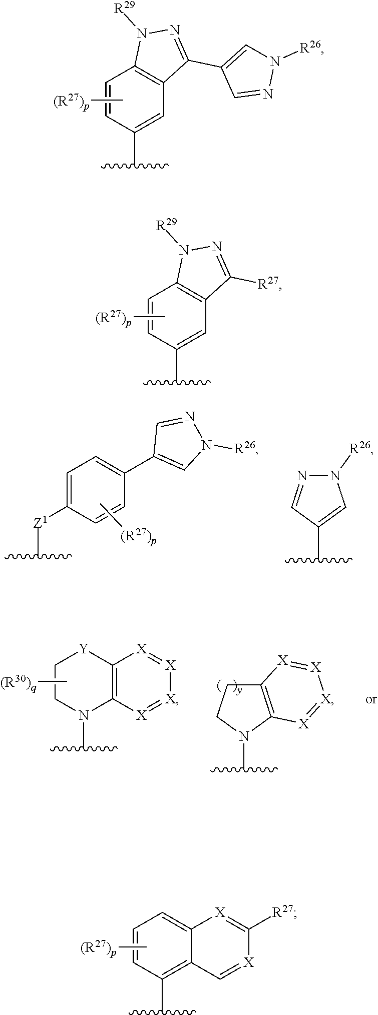

y is 1 or 2; Z is —NR^{c}—, —O—, or —S—;
R^{c} is hydrogen or substituted or unsubstituted C₁-C₆alkyl;
R²⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each X is independently —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen or substituted or unsubstituted C₁-C₆alkyl;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
Y is —C(R³⁰)₂— or N(R²⁸)—;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂; and
q is 0, 1, 2, 3, or 4;
or R³² is -L-C;
L is substituted or unsubstituted C₁-C₆alkyl or substituted or unsubstituted C₁-C₆heteroalkyl;
C is substituted or unsubstituted C₃-C₈cycloalkyl, substituted or unsubstituted 3- to 8-membered heterocycloalkyl, substituted or unsubstituted aryl, or substituted or unsubstituted heteroaryl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, the moiety that occupies the BC Loop region of the bromodomain of CBP/p300 is R³², wherein:
R³² is

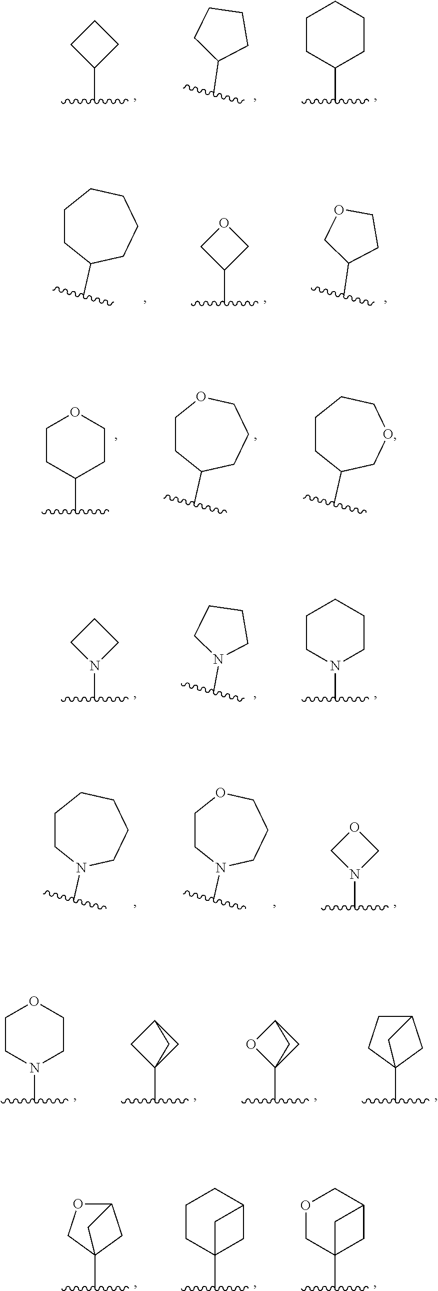

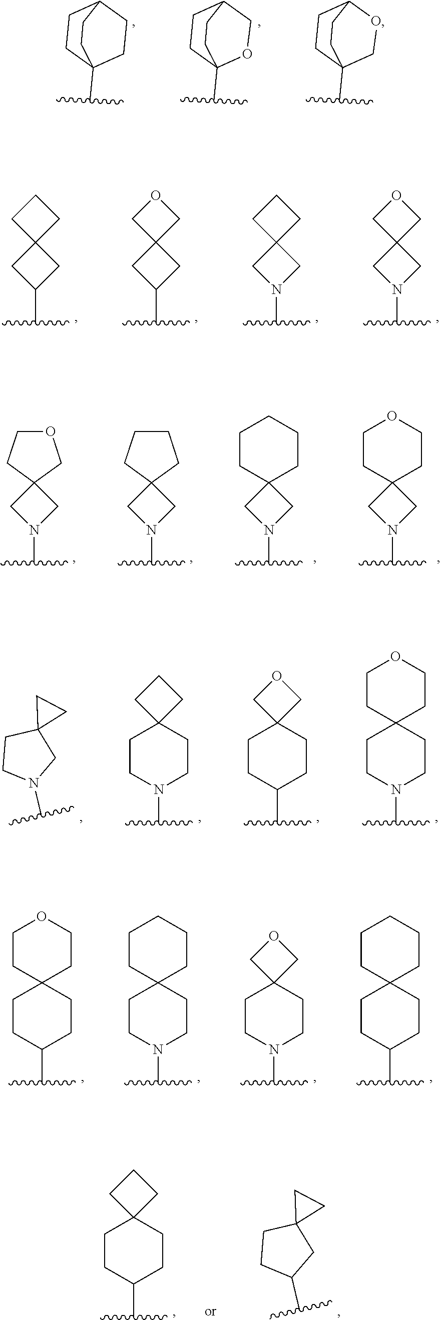

or each of which is substituted or unsubstituted.

In some embodiments, the moiety comprising an acetyl-lysine mimetic comprises

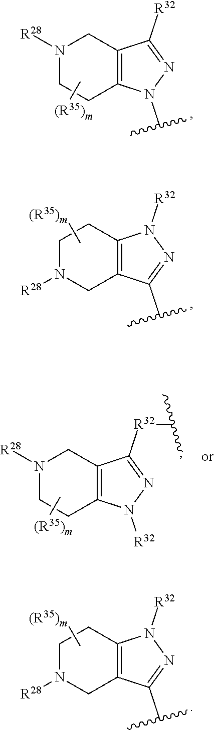

In some embodiments, the moiety comprising an acetyl-lysine mimetic comprises

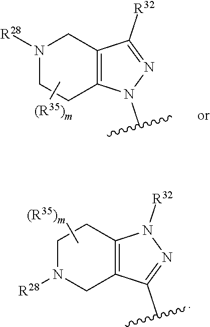

In some embodiments, the moiety comprising an acetyl-lysine mimetic comprises

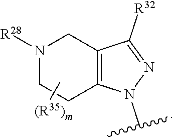

In some embodiments, the moiety comprising an acetyl-lysine mimetic comprises

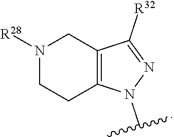

In some embodiments, R³² is

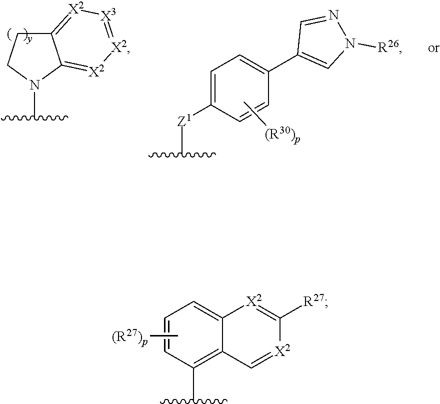

R²⁷ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, substituted or unsubstituted heteroaryl, —CN, —OH, or —OR^{a};
each R³⁰ is independently hydrogen, substituted or unsubstituted C₁-C₆alkyl, substituted or unsubstituted C₁-C₆fluoroalkyl, substituted or unsubstituted C₁-C₆heteroalkyl, —CN, —OH, or —OR^{a}; and
each R^{a} is independently substituted or unsubstituted C₁-C₆alkyl.

In some embodiments, each R²⁷ is independently hydrogen, —CH₃, —CH₂CH₃, —F, —CHF₂, —CF₃, —CN, —OH, —OCH₃, cyclopropyl, cyclobutyl, oxetanyl, tetrahydrofuranyl, tetrahydropyranyl, phenyl, pyrazolyl, 1-methyl pyrazolyl, pyridinyl, or pyrimidinyl.

In some embodiments, R³² is

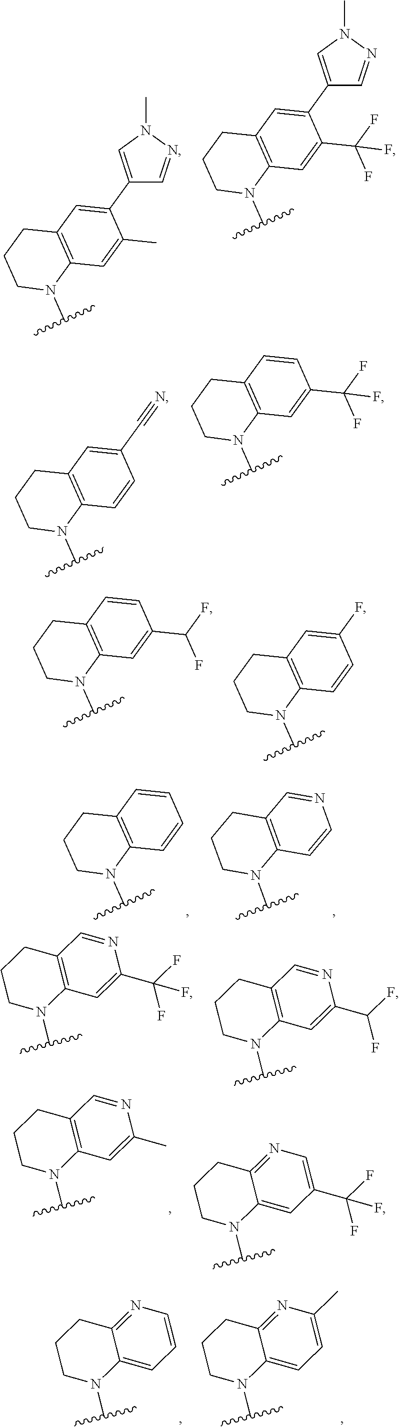

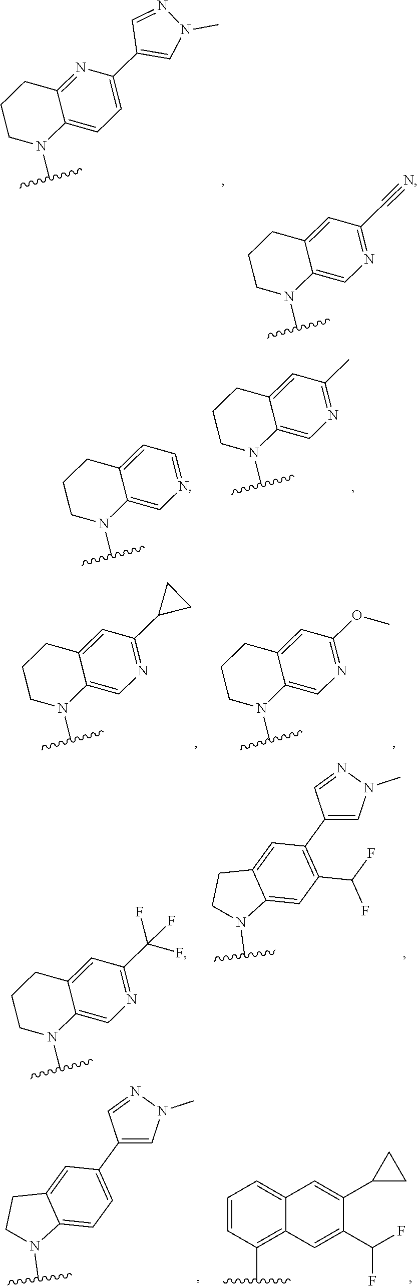

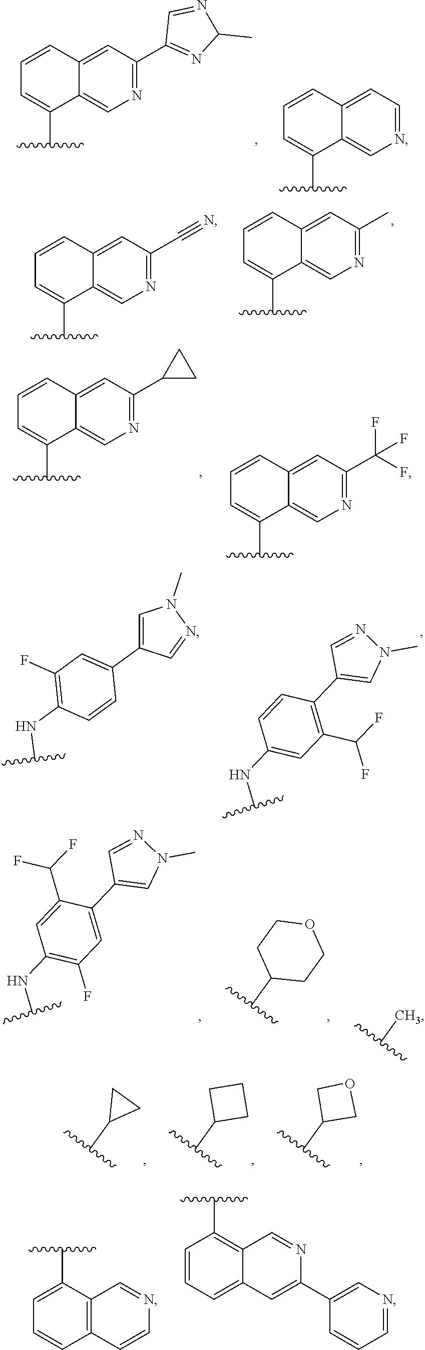

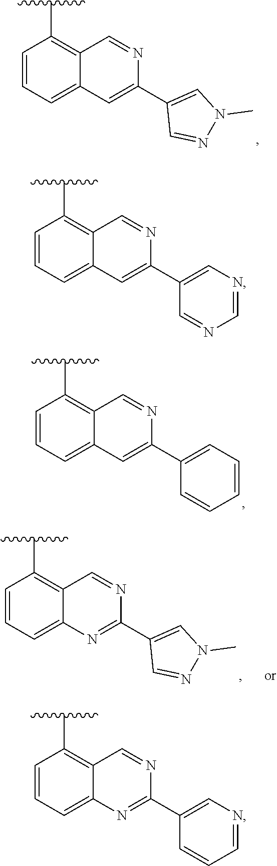 [SMILES: *c1cccc2cc(-c3cnn(C)c3)ncc12 ; *c1cccc2cc(-c3cncnc3)ncc12 ; *c1cccc2cc(-c3ccccc3)ncc12 ; *c1cccc2nc(-c3cnn(C)c3)ncc12 ; *c1cccc2nc(-c3cccnc3)ncc12]

In some embodiments, the moiety comprising an acetyl-lysine mimetic comprises

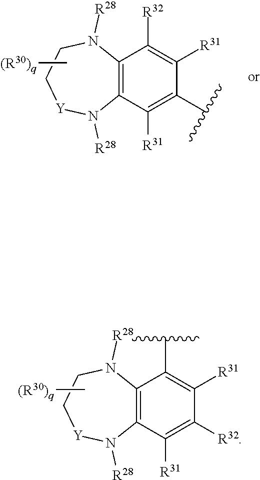

In some embodiments, the moiety comprising an acetyl-lysine mimetic comprises

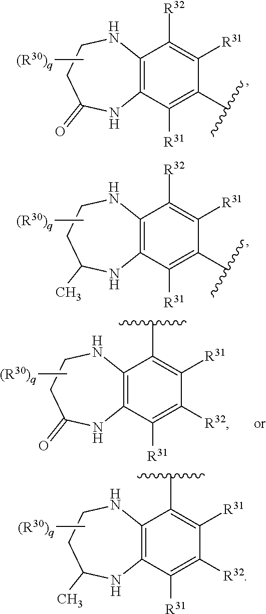

In some embodiments, the moiety comprising an acetyl-lysine mimetic comprises

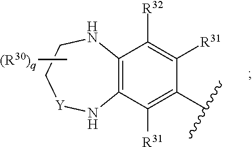

wherein:
each R²⁸ is independently hydrogen or substituted or unsubstituted C₁-C₆alkyl;
R³² is

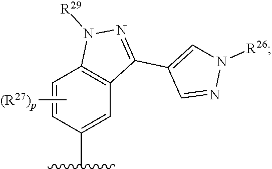

and
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂.

In some embodiments, R³² is

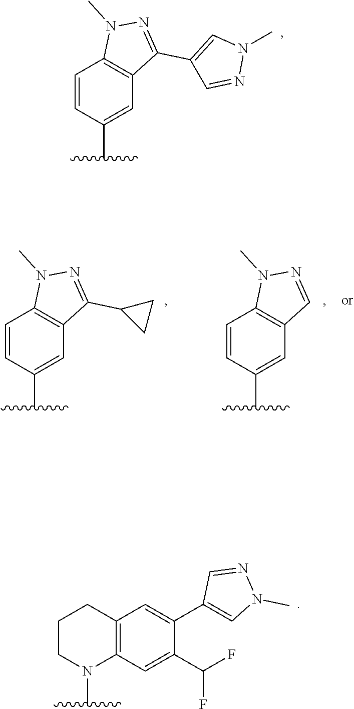 [SMILES: *c1ccc2c(c1)c(-c1cnn(C)c1)nn2C ; *c1ccc2c(c1)c(C1CC1)nn2C ; *c1ccc2c(cnn2C)c1 ; *N1CCCc2cc(-c3cnn(C)c3)c(C(F)F)cc21]

In some embodiments, the moiety comprising an acetyl-lysine mimetic comprises

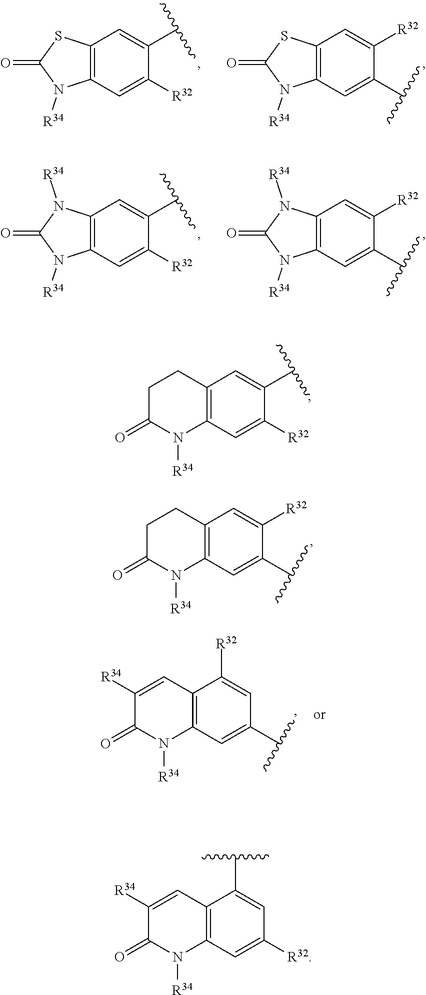

In some embodiments, the moiety comprising an acetyl-lysine mimetic comprises

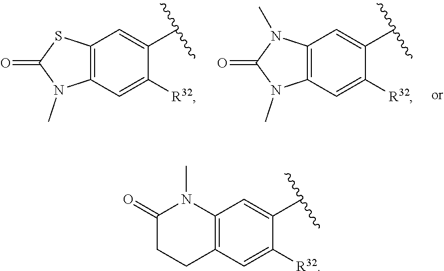

In some embodiments, R³² is

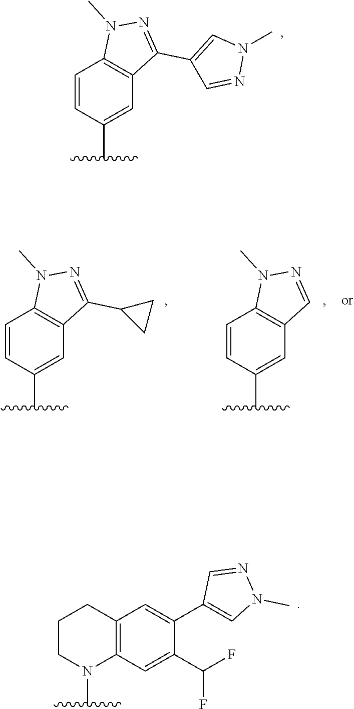 [SMILES: *c1ccc2c(c1)c(-c1cnn(C)c1)nn2C ; *c1ccc2c(c1)c(C1CC1)nn2C ; *c1ccc2c(cnn2C)c1 ; *N1CCCc2cc(-c3cnn(C)c3)c(C(F)F)cc21]

In some embodiments, the moiety comprising an acetyl-lysine mimetic comprises:

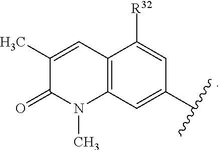

In some embodiments, R³² is

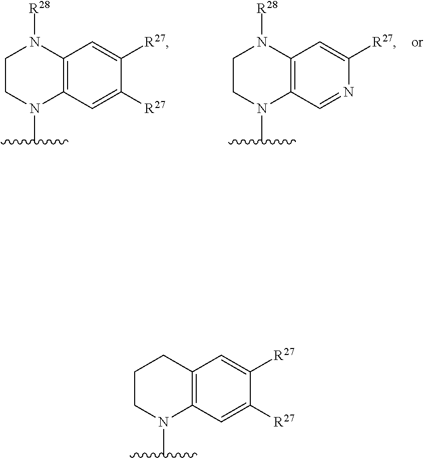

In some embodiments, R³² is

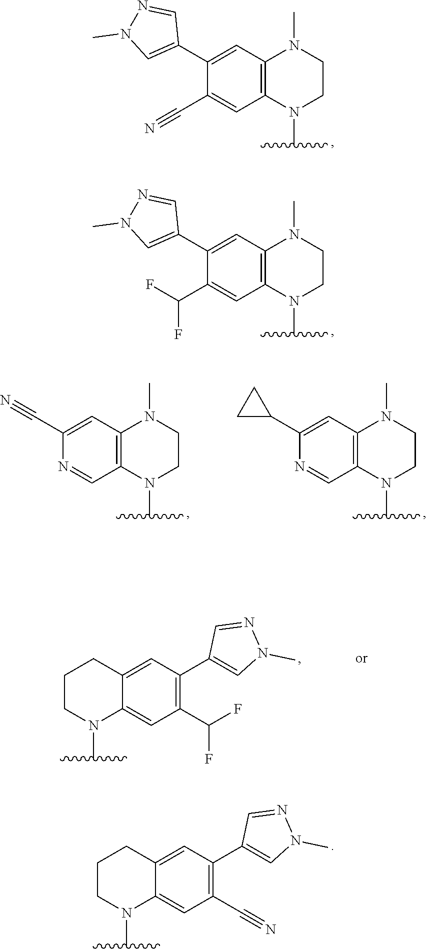 [SMILES: *N1CCN(C)c2cc(-c3cnn(C)c3)c(C#N)cc21 ; *N1CCN(C)c2cc(-c3cnn(C)c3)c(C(F)F)cc21 ; *N1CCN(C)c2cc(C#N)ncc21 ; *N1CCN(C)c2cc(C3CC3)ncc21 ; *N1CCCc2cc(-c3cnn(C)c3)c(C(F)F)cc21 ; *N1CCCc2cc(-c3cnn(C)c3)c(C#N)cc21]

In some embodiments, the moiety comprising an acetyl-lysine mimetic comprises

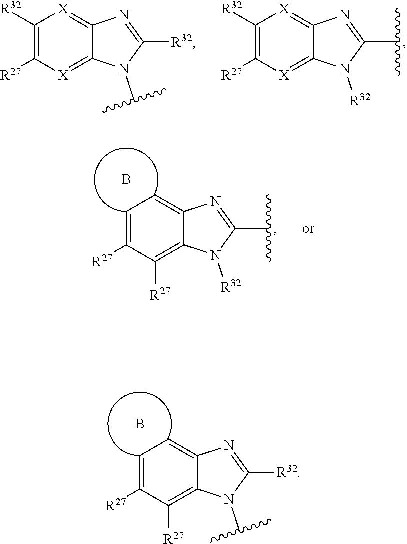

In some embodiments, the moiety comprising an acetyl-lysine mimetic comprises the moiety comprising an acetyl-lysine mimetic comprises

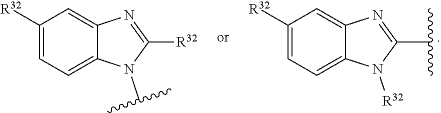

In some embodiments,

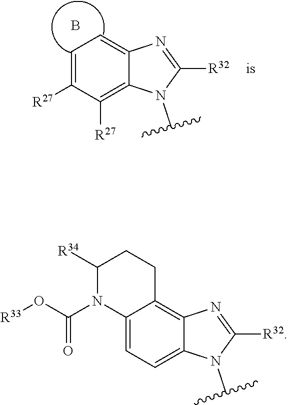

In some embodiments, the moiety comprising an acetyl-lysine mimetic comprises

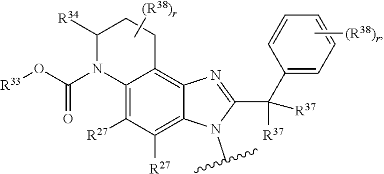

wherein:
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR^{a}, or —N(R^{b})₂;
R³³ is hydrogen, substituted or unsubstituted C₁-C₆alkyl, substituted or unsubstituted C₁-C₆fluoroalkyl, or substituted or unsubstituted C₁-C₆heteroalkyl;
R³⁴ is hydrogen or substituted or unsubstituted C₁-C₆alkyl;
each R³⁸ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, —CN, —NO₂, —OH, —OR^{a}, —OC(═O)R^{a}, —OC(═O)N(R^{b})₂, —OC(═O)OR^{a}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
r is 0, 1, 2, 3, or 4.

In some embodiments,

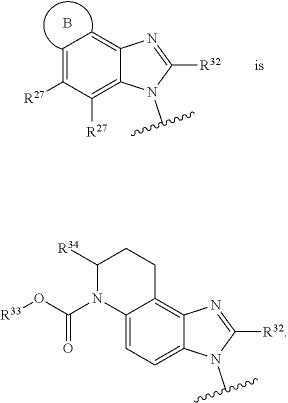

In some embodiments, the moiety comprising an acetyl-lysine mimetic comprises

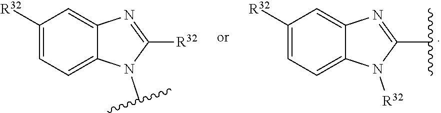

In some embodiments, R³² is

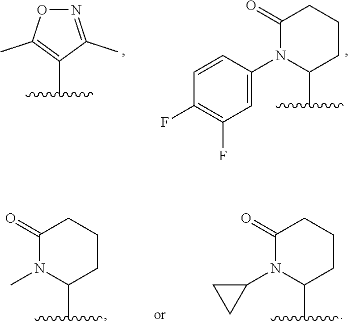

In some embodiments, R³² is

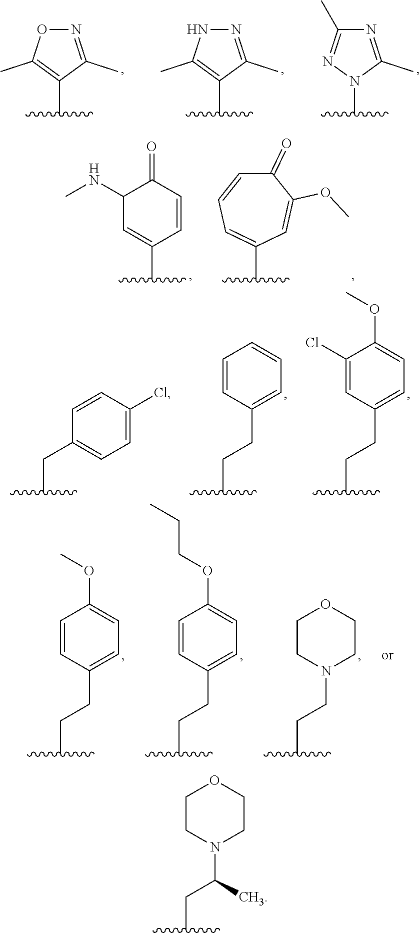

In some embodiments, the acetyl-lysine mimetic moiety that binds in the acetyl-lysine (KAc) binding site of the bromodomain of CBP/p300 comprises 1-(1,4,6,7-tetrahydro-5H-pyrazolo[4,3-c]pyridin-5-yl)ethan-1-one; or N-methyl-1,4,6,7-tetrahydro-5H-pyrazolo[4,3-c]pyridine-5-carboxamide; wherein the acetyl-lysine mimetic moiety optionally further comprises:
1) a moiety at the 1-position of the 1,4,6,7-tetrahydro-5H-pyrazolo[4,3-c]pyridin-5-yl group that occupies the lipophilic shelf (LPF) region of the bromodomain of CBP/p300; or
2) a moiety that at the 3-position of the 1,4,6,7-tetrahydro-5H-pyrazolo[4,3-c]pyridin-5-yl group occupies the BC Loop region of the bromodomain of CBP/p300; or
3) both 1) and 2).

In some embodiments, the acetyl-lysine mimetic moiety that binds in the acetyl-lysine (KAc) binding site of the bromodomain of CBP/p300 has the structure:

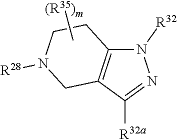

R²⁸ is —C(═O)CH₃, —C(═O)CH₂CH₃, —C(═O)NH₂, —C(═O)NH(CH₃); or —C(═O)NH(CH₂CH₃);
R³² is a moiety that occupies the BC Loop region of the bromodomain of CBP/p300;
R^{32a} is a moiety that occupies the lipophilic shelf (LPF) region of the bromodomain of CBP/p300;
each R³⁵ is independently hydrogen, C₁-C₄alkyl, or C₁-C₄fluoroalkyl;
m is 0, 1, 2, 3, or 4;
L is an optional linker;
wherein L is covalently attached to the R³² group, or at the position occupied by R³², or the R^{32a} group.

In some embodiments, R³² is hydrogen, substituted or unsubstituted C₁-C₆alkyl, substituted or unsubstituted C₃-C₁₂cycloalkyl, or substituted or unsubstituted 3- to 12-membered heterocycloalkyl;
R^{32a} is

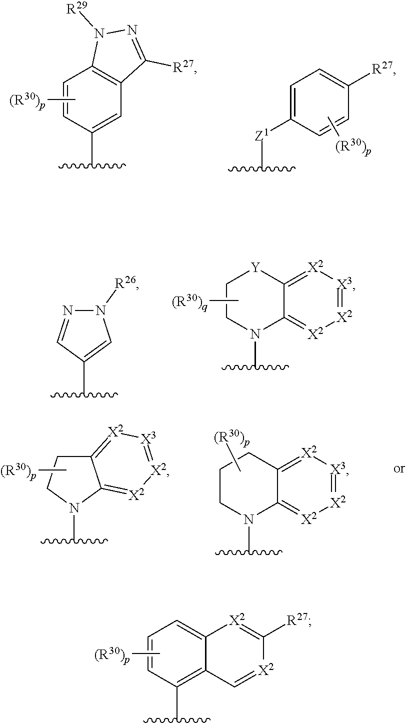

at least one X² is —CR³⁰— and at most two X² are —N—;
Z¹ is —NR^{c}— or —O—;
R^{c} is hydrogen or C₁-C₆alkyl;
R²⁶ is hydrogen, substituted or unsubstituted C₁-C₄alkyl, C₁-C₄fluoroalkyl, substituted or unsubstituted C₃-C₈cycloalkyl, or substituted or unsubstituted 3- to 8-membered heterocycloalkyl;
X³ is —CR²⁷— or —N—;
R²⁷ is hydrogen, halogen, substituted or unsubstituted C₁-C₄alkyl, C₁-C₄fluoroalkyl, substituted or unsubstituted C₃-C₈cycloalkyl, substituted or unsubstituted 3- to 8-membered heterocycloalkyl, substituted or unsubstituted phenyl, substituted or unsubstituted monocyclic heteroaryl, —CN, —OH, —OR^{a}, —N(R^{b})₂, —NR^{b}C(═O)R^{a}, —C(═O)R^{b}, —C(═O)OR^{b}, or —C(═O)N(R^{b})₂;
or R²⁷ is

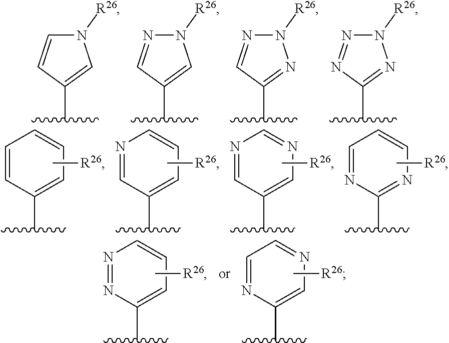

p is 0, 1, 2, or 3;
R²⁹ is hydrogen, substituted or unsubstituted C₁-C₄alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
Y is —C(R³⁰)₂— or N(R²⁸)—;
each R³⁰ is independently hydrogen, halogen, C₁-C₄alkyl, C₁-C₄fluoroalkyl, substituted or unsubstituted C₁-C₆heteroalkyl, —CN, —OH, or —OR^{a};
q is 0, 1, 2, 3, or 4;
each R^{a} is independently C₁-C₄alkyl, C₁-C₆fluoroalkyl, substituted or unsubstituted C₃-C₆cycloalkyl, substituted or unsubstituted monocyclic 3- to 6-membered heterocycloalkyl, substituted or unsubstituted phenyl, or substituted or unsubstituted monocyclic heteroaryl; and
each R^{b} is independently hydrogen, C₁-C₄alkyl, C₁-C₄fluoroalkyl, substituted or unsubstituted C₃-C₆cycloalkyl, substituted or unsubstituted monocyclic 3- to 6-membered heterocycloalkyl, substituted or unsubstituted phenyl, or substituted or unsubstituted monocyclic heteroaryl;
or two R^{b} on the same N atom are taken together with the N atom to which they are attached to form a substituted or unsubstituted N-containing heterocycle.

In some embodiments, R²⁷ is hydrogen, halogen, C₁-C₄alkyl, C₁-C₄fluoroalkyl, substituted or unsubstituted C₃-C₈cycloalkyl, substituted or unsubstituted 3- to 8-membered heterocycloalkyl, substituted or unsubstituted phenyl, substituted or unsubstituted monocyclic 5- or 6-membered heteroaryl, —CN, —OH, —OR^{a}, —N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
or R²⁷ is

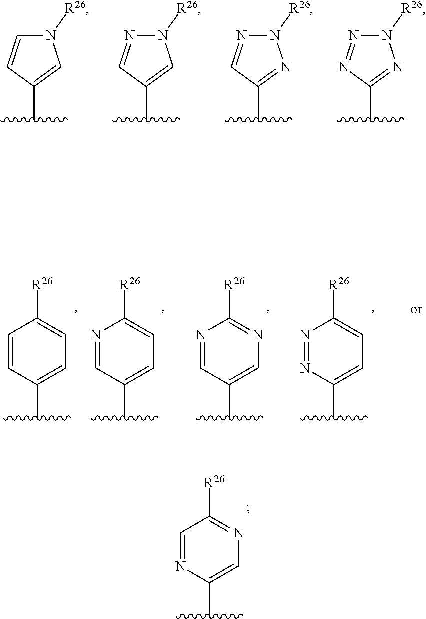

In some embodiments, R³² is

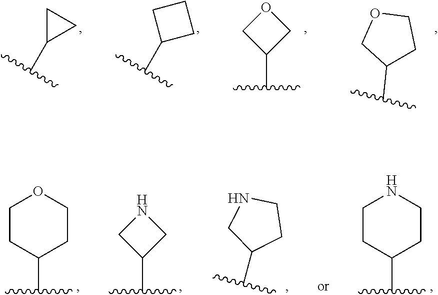

each of which is unsubstituted or substituted with F, Cl, Br, —CH₃, —CD₃, —CH₂CH₃, —CH₂F, —CHF₂, —CF₃, —CH₂CH₂F, —CH₂CHF₂, —CH₂CF₃, —CN, —C(═O)CH₃, —C(═O)CH₂CH₃, —C(═O)CH₂F, —C(═O)CHF₂, —C(═O)CF₃, —C(═O)CH₂CH₂F, —C(═O)CH₂CHF₂, —C(═O)CH₂CF₃, —C(═O)CD₃, or —SO₂CH₃, —SO₂CH₂CH₃, —SO₂CD₃, or —SO₂CH₂CD₃.

In some embodiments, R^{32a} is

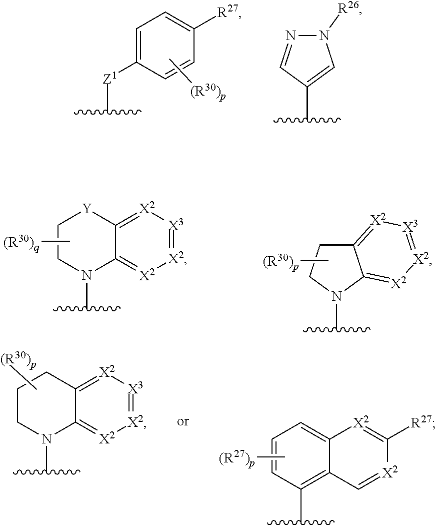

at least one X² is —CR³⁰— and at most two X² are —N—; each R²⁷ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, substituted or unsubstituted heteroaryl, —CN, —OH, or —OR^{a}; and each R^{a} is independently substituted or unsubstituted C₁-C₆alkyl.

In some embodiments, R²⁷ is hydrogen, —CH₃, —CH₂CH₃, —CH₂CH₂CH₃, —CH₂(CH₃)₂, —(CH₃)₃, —F, —CHF₂, —CF₃, —CN, —OH, —OCH₃, cyclopropyl, cyclobutyl, oxetanyl, tetrahydrofuranyl, tetrahydropyranyl, unsubstituted or substituted phenyl, unsubstituted or substituted pyrazolyl, unsubstituted or substituted pyridinyl, or unsubstituted or substituted pyrimidinyl; or
R²⁷ is

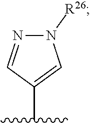

### R³² is

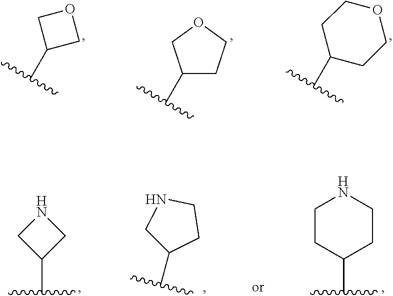 [SMILES: *C1COC1 ; *C1CCOC1 ; *C1CCOCC1 ; *C1CNC1 ; *C1CCNC1 ; *C1CCNCC1]

each of which is unsubstituted or substituted with F, Cl, Br, —CH₃, —CD₃, —CH₂CH₃, —CH₂F, —CHF₂, —CF₃, CN, —C(═O)CH₃, —C(═O)CH₂F, —C(═O)CHF₂, —C(═O)CF₃, or —C(═O)CD₃.

In some embodiments, R³² is

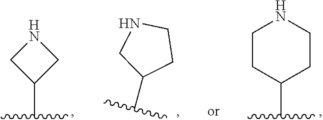 [SMILES: *C1CNC1 ; *C1CCNC1 ; *C1CCNCC1]

wherein the optional linker is covalently attached to the nitrogen of R³² group; or R³² is absent and L is covalently attached to the acetyl-lysine mimetic moiety at the position occupied by R³²; and
R^{32a} is

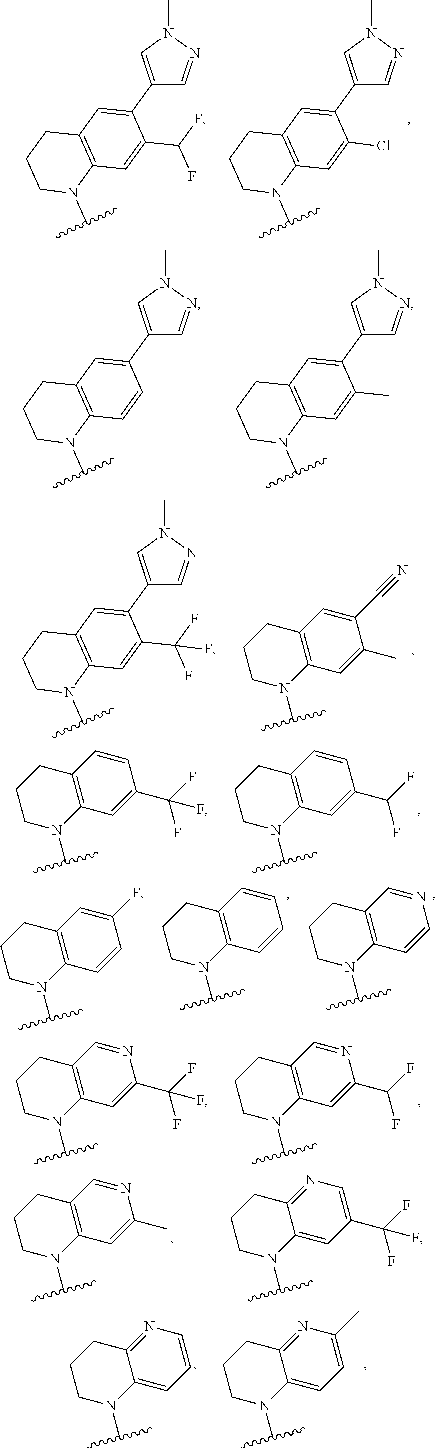

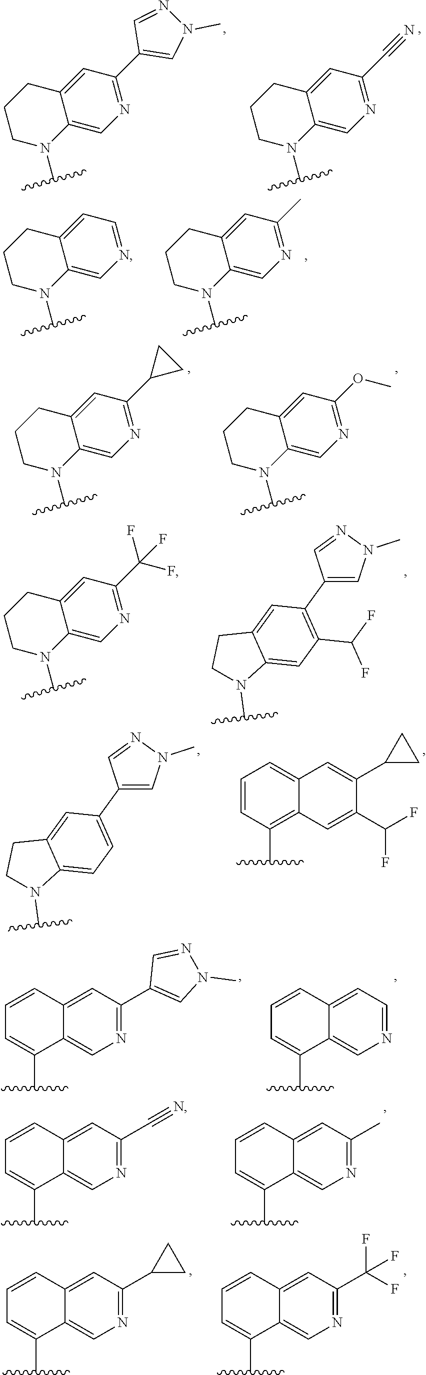

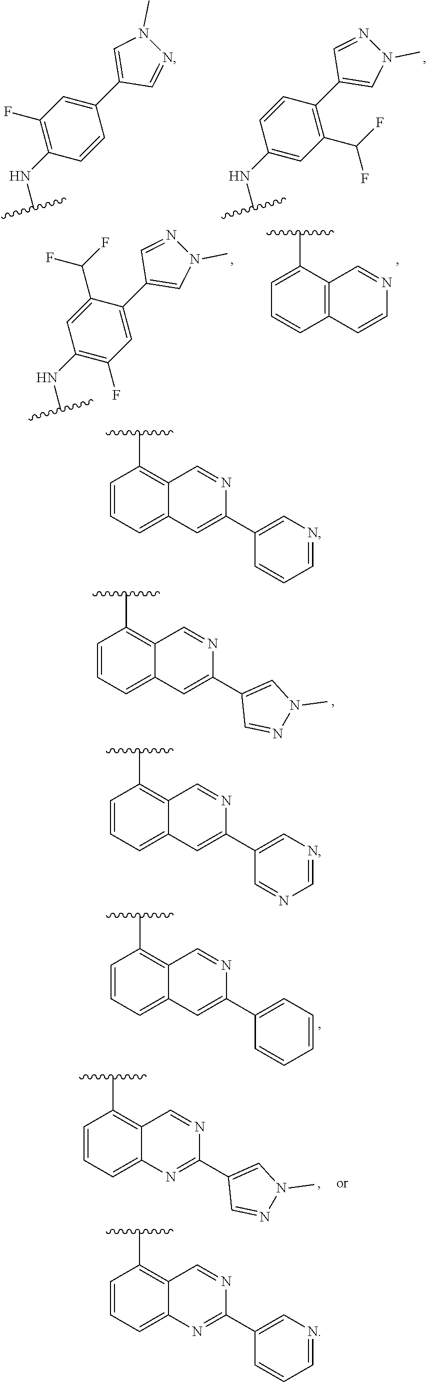

In some embodiments, R³² is

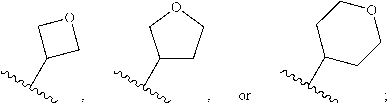 [SMILES: *C1COC1 ; *C1CCOC1 ; *C1CCOCC1]

R^{32a} is

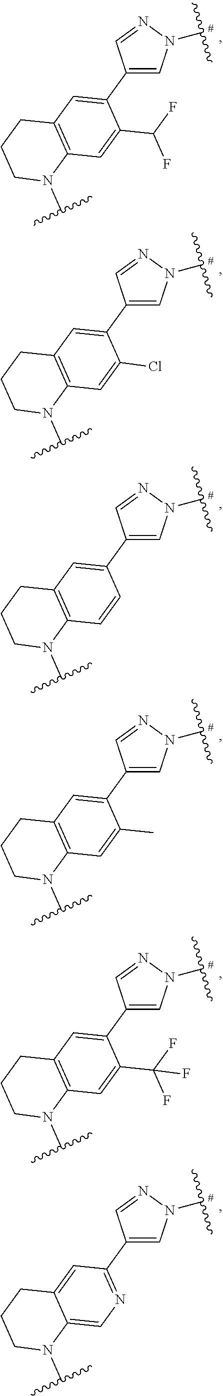 [SMILES: *N1CCCc2cc(-c3cnn(*)c3)c(C(F)F)cc21 ; *N1CCCc2cc(-c3cnn(*)c3)c(Cl)cc21 ; *N1CCCc2cc(-c3cnn(*)c3)ccc21 ; *N1CCCc2cc(-c3cnn(*)c3)c(C)cc21 ; *N1CCCc2cc(-c3cnn(*)c3)c(C(F)(F)F)cc21 ; *N1CCCc2cc(-c3cnn(*)c3)ncc21]

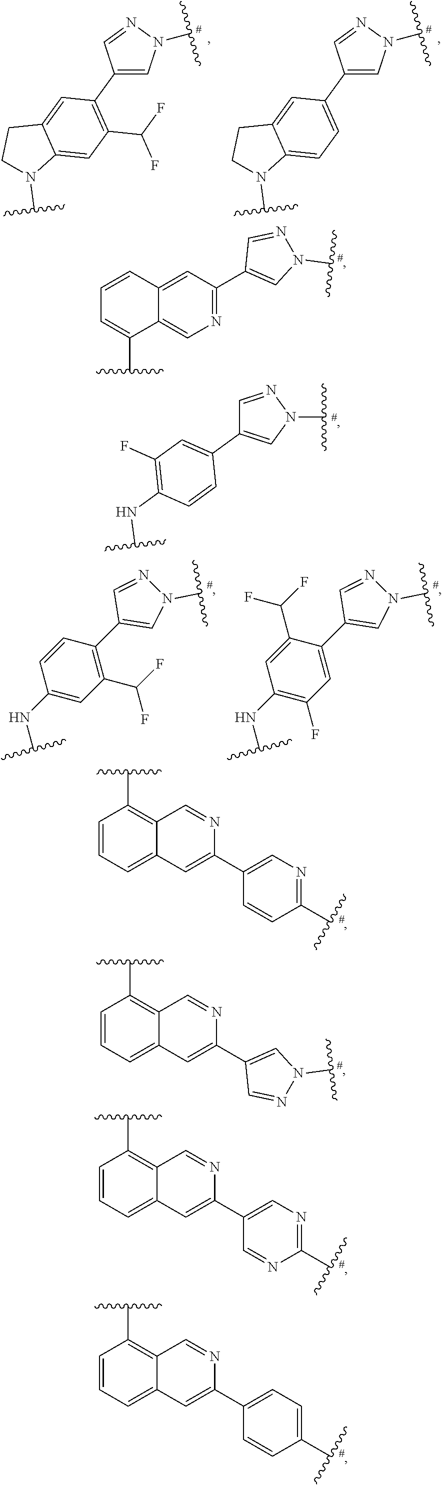

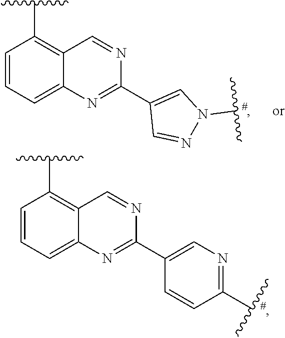 [SMILES: *c1cccc2nc(-c3cnn(*)c3)ncc12 ; *c1ccc(-c2ncc3c(*)cccc3n2)cn1]

wherein
[symbol] is the point of attachment to the optional linker.

In some embodiments, R²⁸ is —C(═O)CH₃ or —C(═O)NH(CH₃); each R³⁵ is independently hydrogen, —CH₃, —CH₂F, —CHF₂, or —CF₃; m is 0, 1, or 2; R³² is

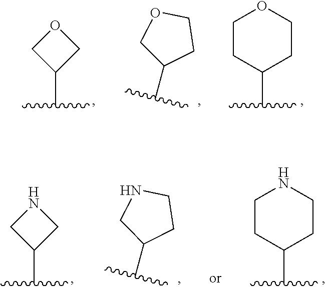

each of which is unsubstituted or substituted with F, —CH₃, —CH₂F, —CHF₂, or —CF₃;
R^{32a} is

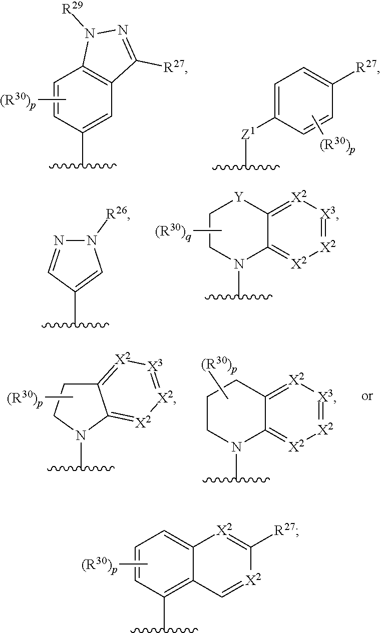

at least one X² is —CR³⁰— and at most two X² are —N—;
   R²⁶ is hydrogen, C₁-C₄alkyl, C₁-C₄fluoroalkyl, substituted or unsubstituted C₃-C₆cycloalkyl, or substituted or unsubstituted 3- to 6-membered heterocycloalkyl;
   R²⁷ is hydrogen, halogen, C₁-C₄alkyl, C₁-C₄fluoroalkyl, substituted or unsubstituted C₃-C₆cycloalkyl, substituted or unsubstituted 3- to 6-membered heterocycloalkyl, substituted or unsubstituted phenyl, substituted or unsubstituted monocyclic 5- or 6-membered heteroaryl, —CN, —OH, —OR^{a}, or —N(R^{b})₂;
   or R²⁷ is

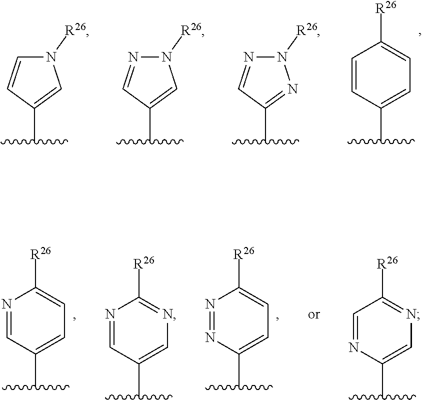

each R³⁰ is independently hydrogen, halogen, C₁-C₄alkyl, C₁-C₄fluoroalkyl, —CN, —OH, or —OR^{a}; R^{a} is C₁-C₄alkyl; each R^{b} is independently hydrogen or C₁-C₆alkyl; wherein L is covalently attached to the R³² group, or at the position occupied by R²⁶, or at the position occupied by R²⁷.

In some embodiments, R³² is

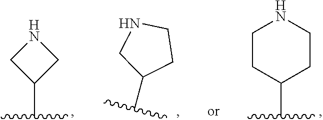 [SMILES: *C1CNC1 ; *C1CCNC1 ; *C1CCNCC1]

wherein the optional linker is covalently attached to the nitrogen of R³² group; or R³² is absent and the optional linker is covalently attached to the acetyl-lysine mimetic moiety at the position occupied by R³²;
R^{32a} is

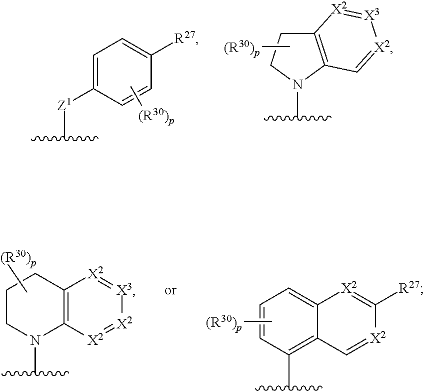

at least one X² is —CR³⁰— and at most two X² are —N—; X³ is —CR²⁷— or —N—; Z¹ is —NH— or —O—;
R²⁶ is hydrogen, C₁-C₄alkyl, C₁-C₄fluoroalkyl, substituted or unsubstituted C₃-C₆cycloalkyl, or substituted or unsubstituted 3- to 6-membered heterocycloalkyl;
R²⁷ is hydrogen, halogen, C₁-C₄alkyl, C₁-C₄fluoroalkyl, substituted or unsubstituted C₃-C₆cycloalkyl, substituted or unsubstituted 3- to 6-membered heterocycloalkyl, substituted or unsubstituted phenyl, substituted or unsubstituted monocyclic 5- or 6-membered heteroaryl, —CN, —OH, —OR^{a}, or —N(R^{b})₂;
or R²⁷ is

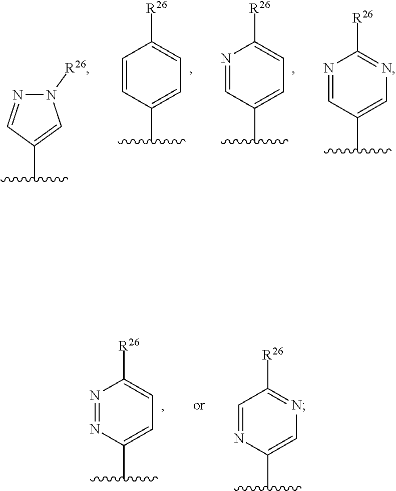

and
each R³⁰ is independently hydrogen, halogen, C₁-C₄alkyl, C₁-C₄fluoroalkyl, —CN, —OH, or —OR^{a}.

In some embodiments, R³² is

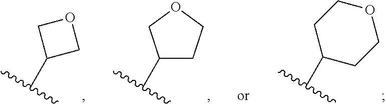 [SMILES: *C1COC1 ; *C1CCOC1 ; *C1CCOCC1]

R^{32a} is

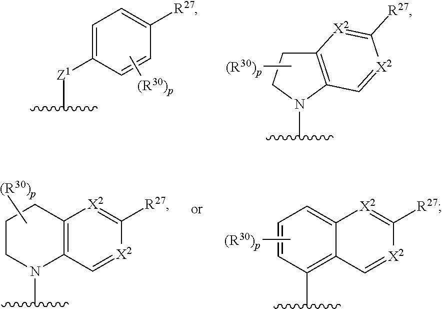

R²⁷ is

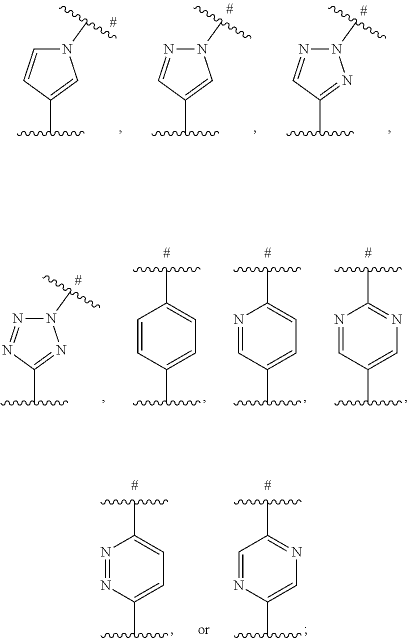

Z¹ is —NH— or —O—; and
[symbol] is the point of attachment to the optional linker.

In some embodiments, SB-CBP/p300 has the structure of Formula (IIIb):

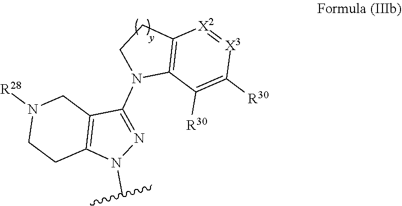

wherein:
each X² is independently —CR³⁰— or —N—;
X³ is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂; and
each R³⁰ is independently hydrogen, halogen, C₁-C₄alkyl, C₁-C₄fluoroalkyl, —CN, —OH, or —OR^{a}; y is 1 or 2;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl; and
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIIc):

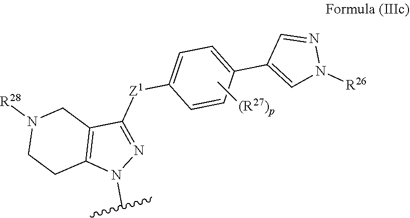

wherein:
R²⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{a}, or —C(═O)N(R^{b})₂;
Z¹ is —NR^{c}—, —O—, or —S—;
R^{c} is hydrogen or substituted or unsubstituted C₁-C₆alkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl; and
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIId-1) or (IIId-2):

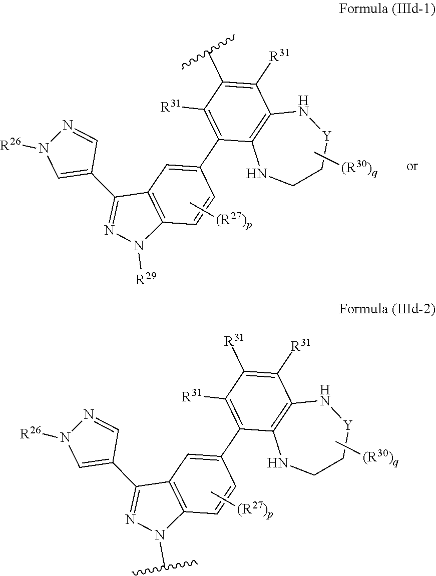

wherein:
R²⁶ is hydrogen, substituted or unsubstituted C₁-C₆alkyl, substituted or unsubstituted C₁-C₆heteroalkyl, substituted or unsubstituted C₃-C₈cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
each R³¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)NR^{b}, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
Y is —C(R³⁰)₂— or C(═O);
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl; and
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIIe):

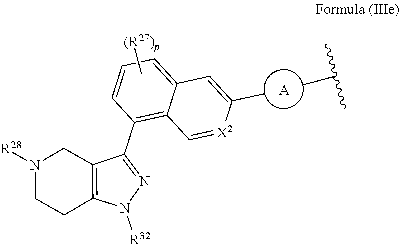

wherein:
X² is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂; R³² is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
Ring A is absent or substituted or unsubstituted C₃-C₈cycloalkyl, substituted or unsubstituted 3- to 8-membered heterocycloalkyl, substituted or unsubstituted aryl, or substituted or unsubstituted heteroaryl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl; and
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has one of the following structures:

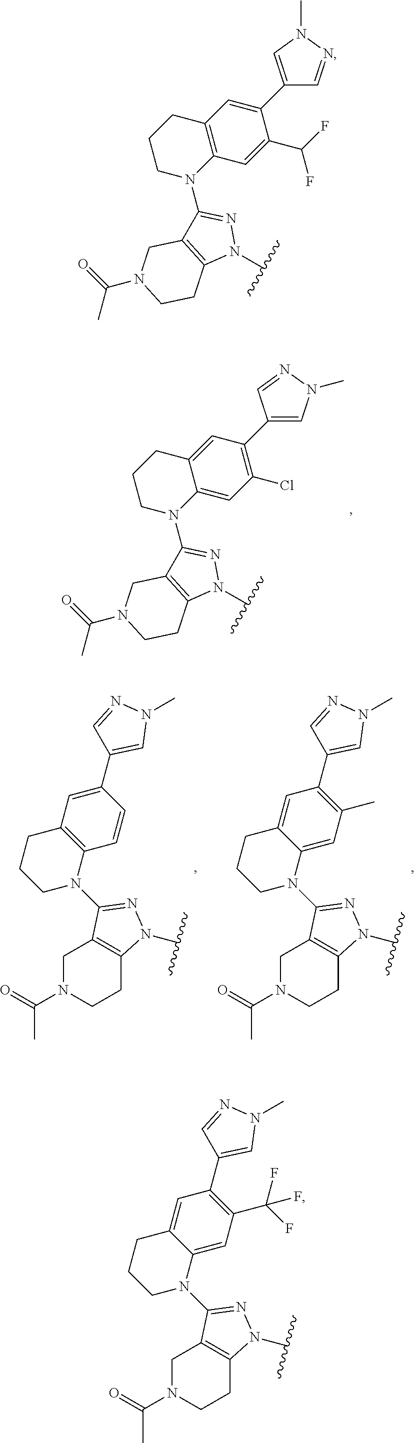 [SMILES: *n1nc(N2CCCc3cc(-c4cnn(C)c4)c(C(F)F)cc32)c2c1CCN(C(C)=O)C2 ; *n1nc(N2CCCc3cc(-c4cnn(C)c4)c(Cl)cc32)c2c1CCN(C(C)=O)C2 ; *n1nc(N2CCCc3cc(-c4cnn(C)c4)ccc32)c2c1CCN(C(C)=O)C2 ; *n1nc(N2CCCc3cc(-c4cnn(C)c4)c(C)cc32)c2c1CCN(C(C)=O)C2 ; *n1nc(N2CCCc3cc(-c4cnn(C)c4)c(C(F)(F)F)cc32)c2c1CCN(C(C)=O)C2]

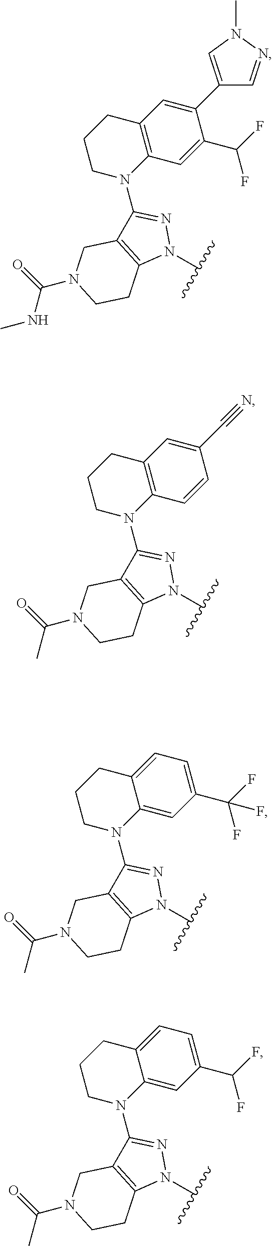 [SMILES: *n1nc(N2CCCc3cc(-c4cnn(C)c4)c(C(F)F)cc32)c2c1CCN(C(=O)NC)C2 ; *n1nc(N2CCCc3cc(C#N)ccc32)c2c1CCN(C(C)=O)C2 ; *n1nc(N2CCCc3ccc(C(F)(F)F)cc32)c2c1CCN(C(C)=O)C2 ; *n1nc(N2CCCc3ccc(C(F)F)cc32)c2c1CCN(C(C)=O)C2]

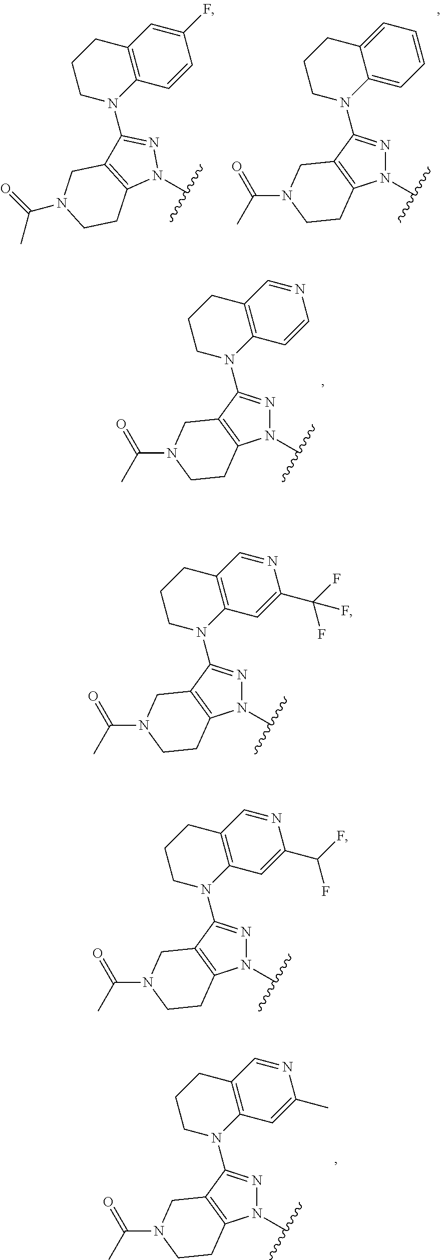 [SMILES: *n1nc(N2CCCc3cc(F)ccc32)c2c1CCN(C(C)=O)C2 ; *n1nc(N2CCCc3ccccc32)c2c1CCN(C(C)=O)C2 ; *n1nc(N2CCCc3cnccc32)c2c1CCN(C(C)=O)C2 ; *n1nc(N2CCCc3cnc(C(F)(F)F)cc32)c2c1CCN(C(C)=O)C2 ; *n1nc(N2CCCc3cnc(C(F)F)cc32)c2c1CCN(C(C)=O)C2 ; *n1nc(N2CCCc3cnc(C)cc32)c2c1CCN(C(C)=O)C2]

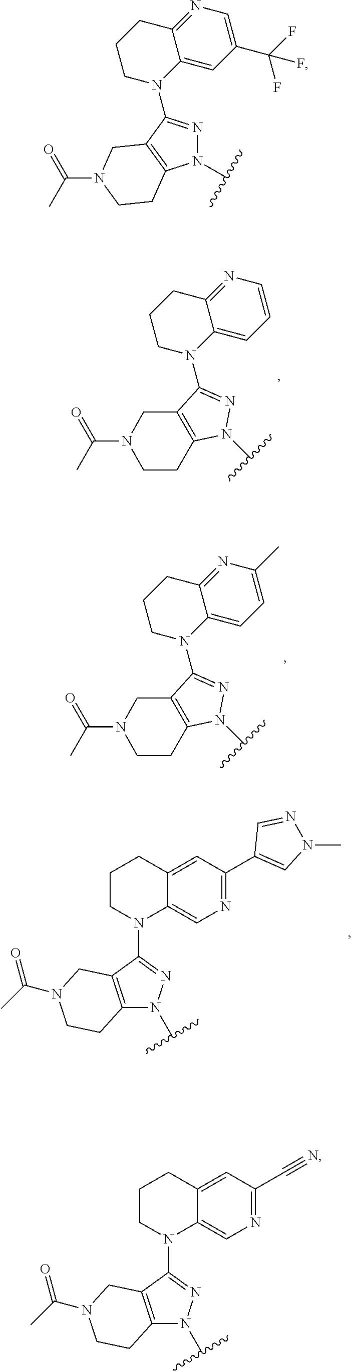

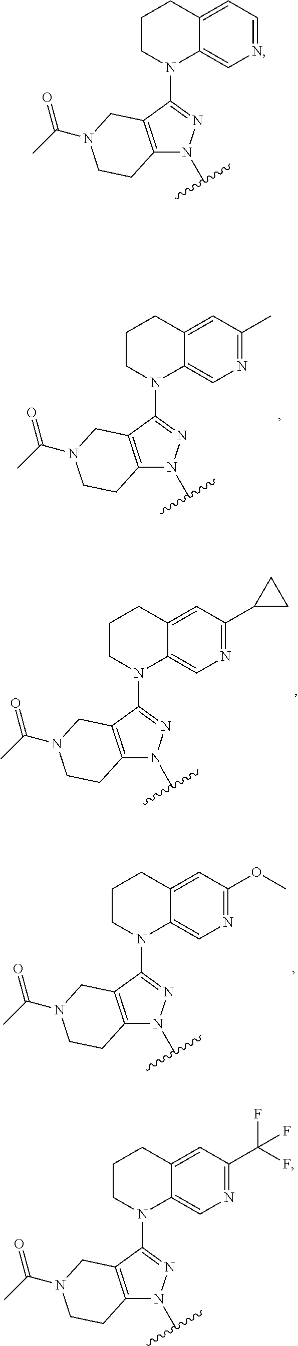

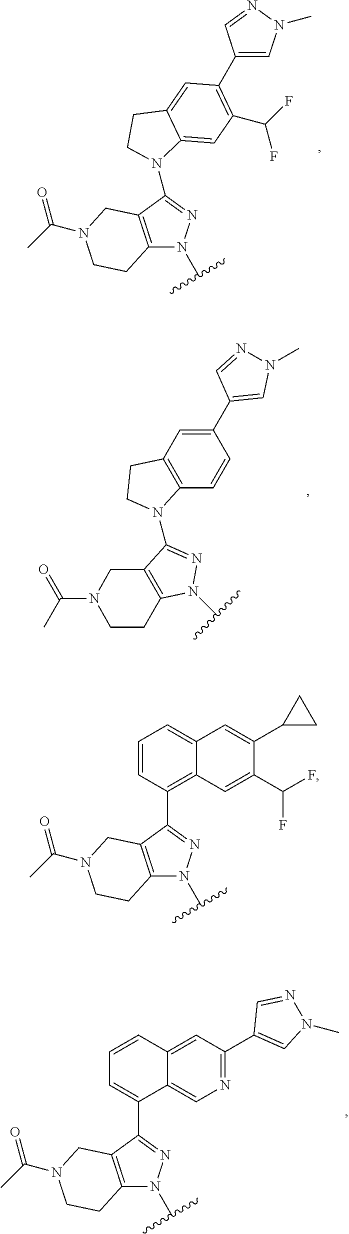

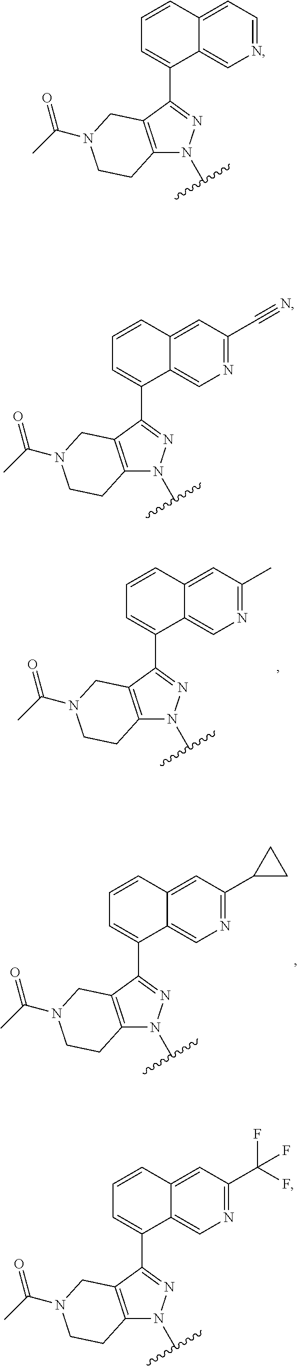

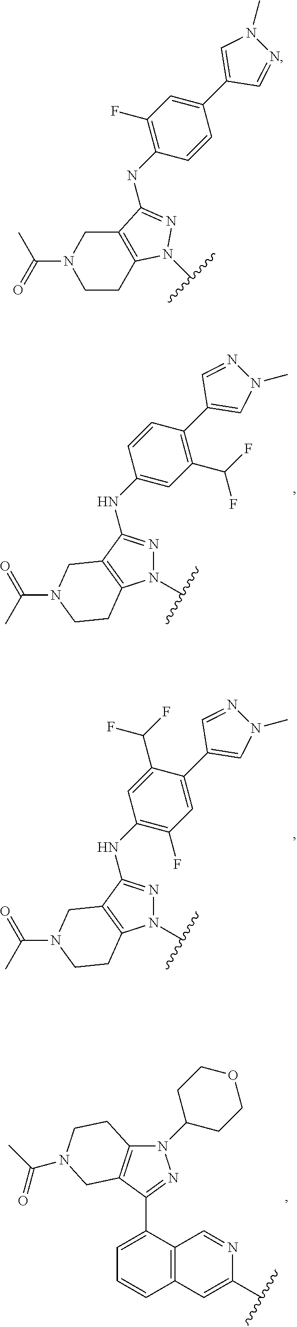

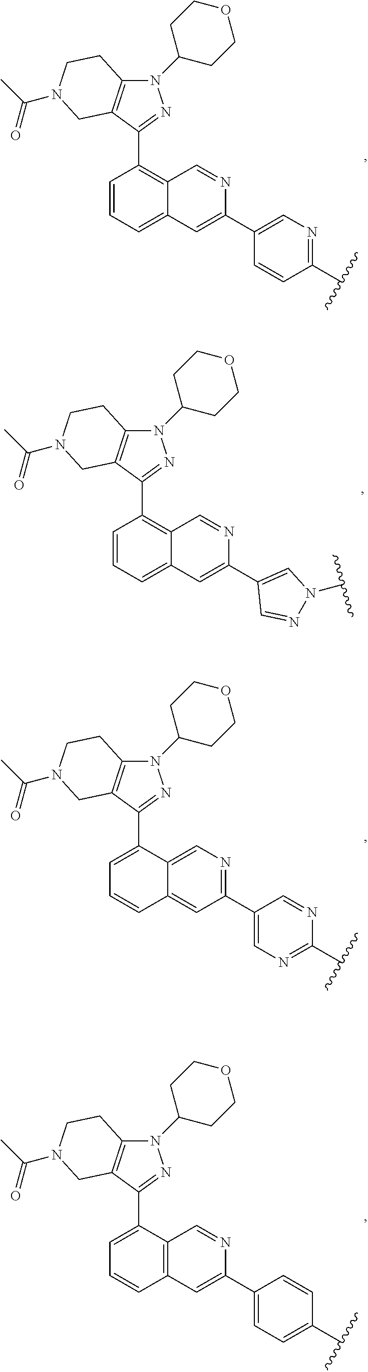 [SMILES: *c1ccc(-c2cc3cccc(-c4nn(C5CCOCC5)c5c4CN(C(C)=O)CC5)c3cn2)cn1 ; *n1cc(-c2cc3cccc(-c4nn(C5CCOCC5)c5c4CN(C(C)=O)CC5)c3cn2)cn1 ; *c1ncc(-c2cc3cccc(-c4nn(C5CCOCC5)c5c4CN(C(C)=O)CC5)c3cn2)cn1 ; *c1ccc(-c2cc3cccc(-c4nn(C5CCOCC5)c5c4CN(C(C)=O)CC5)c3cn2)cc1]

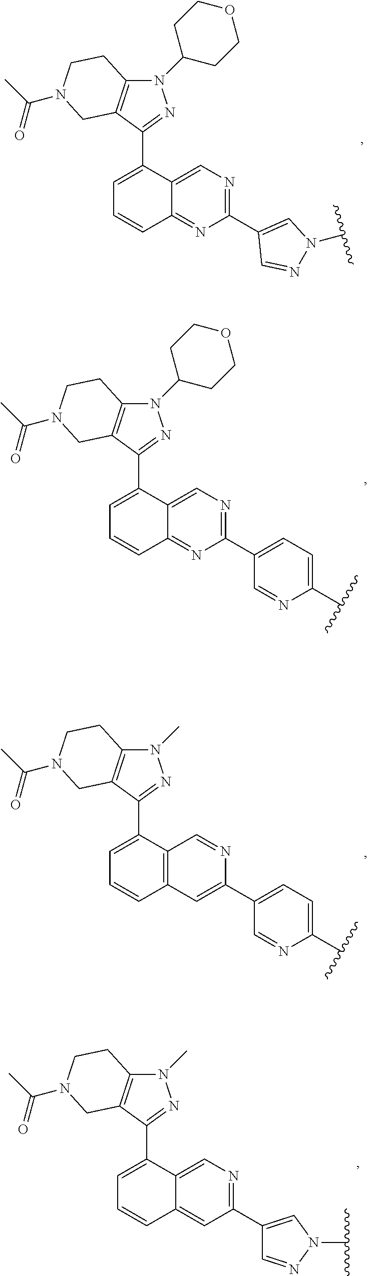 [SMILES: *n1cc(-c2ncc3c(-c4nn(C5CCOCC5)c5c4CN(C(C)=O)CC5)cccc3n2)cn1 ; *c1ccc(-c2ncc3c(-c4nn(C5CCOCC5)c5c4CN(C(C)=O)CC5)cccc3n2)cn1 ; *c1ccc(-c2cc3cccc(-c4nn(C)c5c4CN(C(C)=O)CC5)c3cn2)cn1 ; *n1cc(-c2cc3cccc(-c4nn(C)c5c4CN(C(C)=O)CC5)c3cn2)cn1]

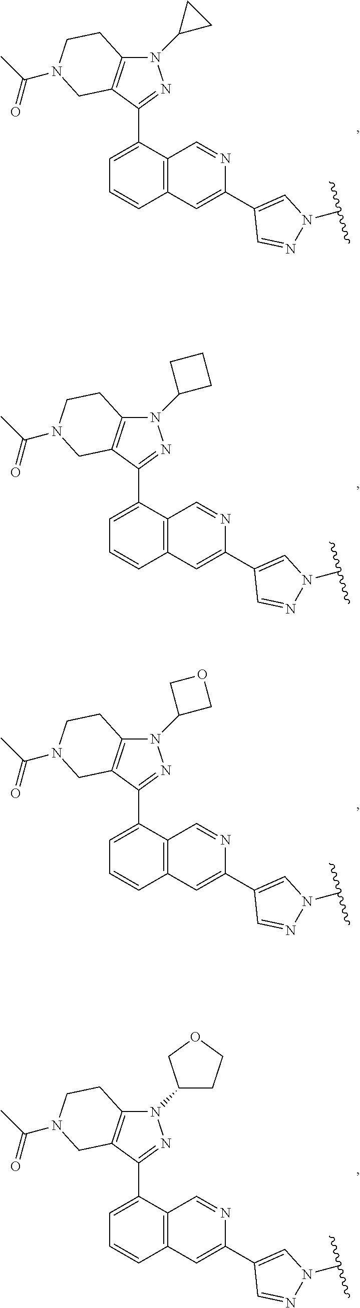 [SMILES: *n1cc(-c2cc3cccc(-c4nn(C5CC5)c5c4CN(C(C)=O)CC5)c3cn2)cn1 ; *n1cc(-c2cc3cccc(-c4nn(C5CCC5)c5c4CN(C(C)=O)CC5)c3cn2)cn1 ; *n1cc(-c2cc3cccc(-c4nn(C5COC5)c5c4CN(C(C)=O)CC5)c3cn2)cn1 ; *n1cc(-c2cc3cccc(-c4nn([C@H]5CCOC5)c5c4CN(C(C)=O)CC5)c3cn2)cn1]

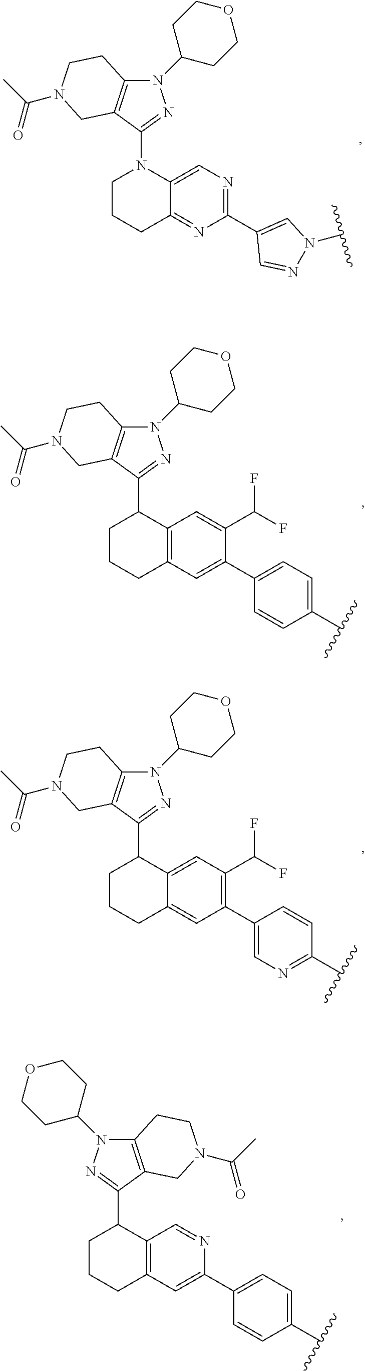 [SMILES: *n1cc(-c2ncc3c(n2)CCCN3c2nn(C3CCOCC3)c3c2CN(C(C)=O)CC3)cn1 ; *c1ccc(-c2cc3c(cc2C(F)F)C(c2nn(C4CCOCC4)c4c2CN(C(C)=O)CC4)CCC3)cc1 ; *c1ccc(-c2cc3c(cc2C(F)F)C(c2nn(C4CCOCC4)c4c2CN(C(C)=O)CC4)CCC3)cn1 ; *c1ccc(-c2cc3c(cn2)C(c2nn(C4CCOCC4)c4c2CN(C(C)=O)CC4)CCC3)cc1]

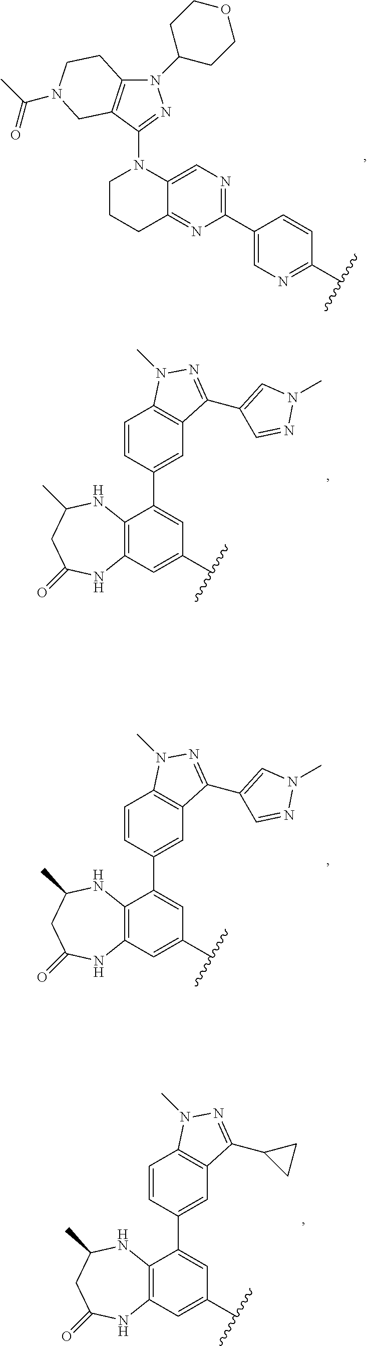 [SMILES: *c1ccc(-c2ncc3c(n2)CCCN3c2nn(C3CCOCC3)c3c2CN(C(C)=O)CC3)cn1 ; *c1cc2c(c(-c3ccc4c(c3)c(-c3cnn(C)c3)nn4C)c1)NC(C)CC(=O)N2 ; *c1cc2c(c(-c3ccc4c(c3)c(-c3cnn(C)c3)nn4C)c1)N[C@H](C)CC(=O)N2 ; *c1cc2c(c(-c3ccc4c(c3)c(C3CC3)nn4C)c1)N[C@H](C)CC(=O)N2]

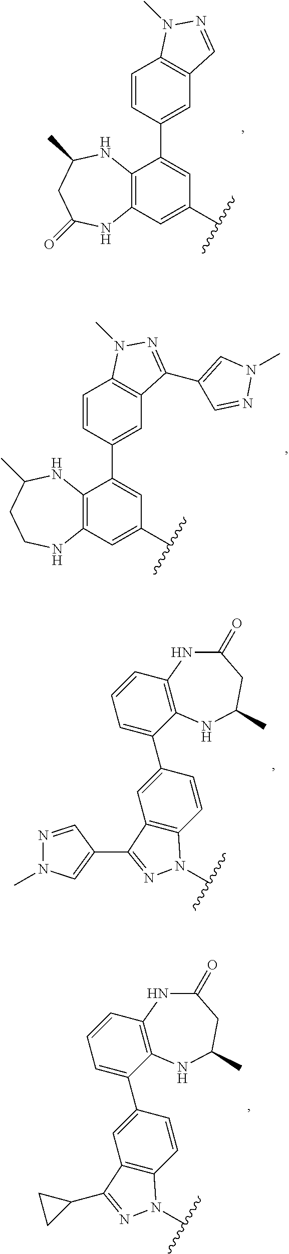

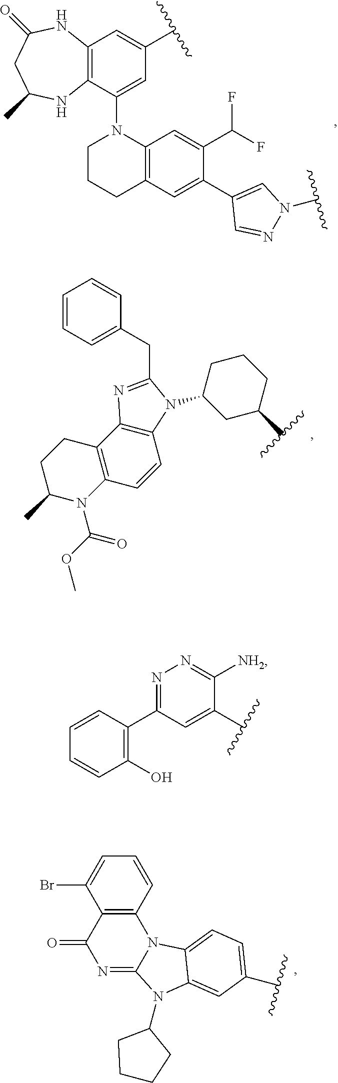 [SMILES: *c1cc2c(c(N3CCCc4cc(-c5cnn(*)c5)c(C(F)F)cc43)c1)N[C@@H](C)CC(=O)N2 ; *[C@@H]1CCC[C@@H](n2c(Cc3ccccc3)nc3c4c(ccc32)N(C(=O)OC)[C@@H](C)CC4)C1 ; *c1cc(-c2ccccc2O)nnc1N ; *c1ccc2c(c1)n(C1CCCC1)c1nc(=O)c3c(Br)cccc3n21]

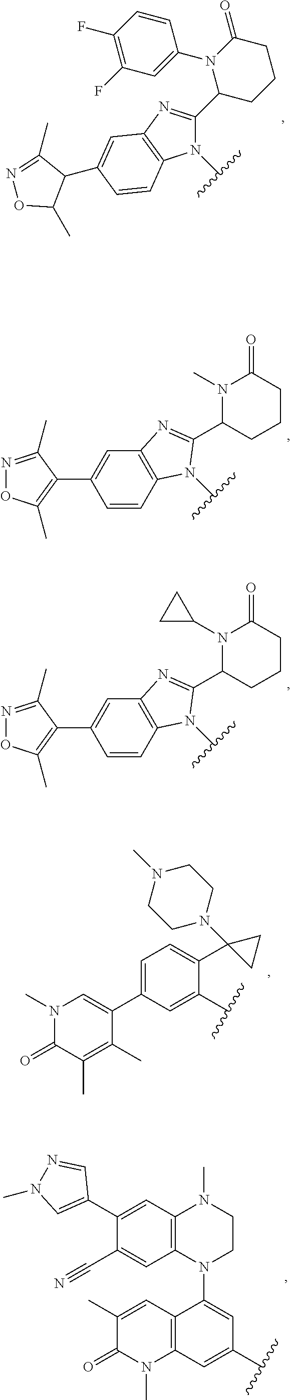 [SMILES: *n1c(C2CCCC(=O)N2c2ccc(F)c(F)c2)nc2cc(C3C(C)=NOC3C)ccc21 ; *n1c(C2CCCC(=O)N2C)nc2cc(-c3c(C)noc3C)ccc21 ; *n1c(C2CCCC(=O)N2C2CC2)nc2cc(-c3c(C)noc3C)ccc21 ; *c1cc(-c2cn(C)c(=O)c(C)c2C)ccc1C1(N2CCN(C)CC2)CC1 ; *c1cc(N2CCN(C)c3cc(-c4cnn(C)c4)c(C#N)cc32)c2cc(C)c(=O)n(C)c2c1]

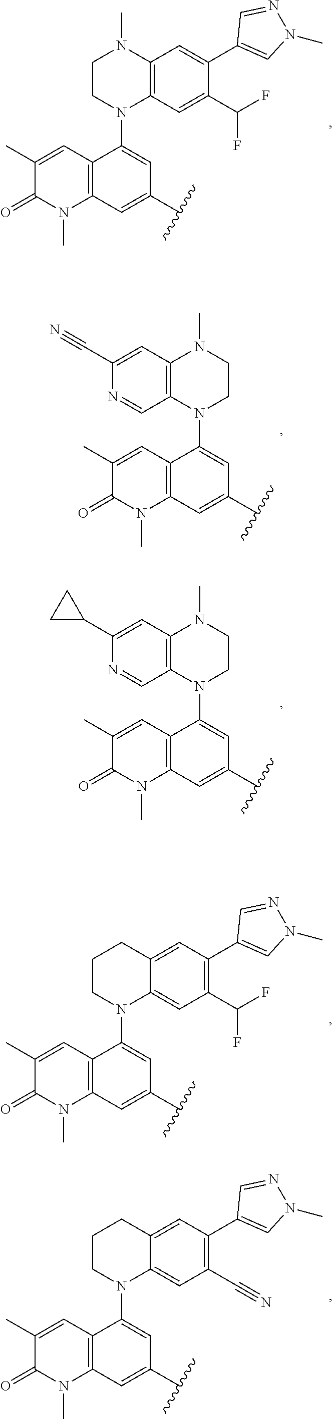 [SMILES: *c1cc(N2CCN(C)c3cc(-c4cnn(C)c4)c(C(F)F)cc32)c2cc(C)c(=O)n(C)c2c1 ; *c1cc(N2CCN(C)c3cc(C#N)ncc32)c2cc(C)c(=O)n(C)c2c1 ; *c1cc(N2CCN(C)c3cc(C4CC4)ncc32)c2cc(C)c(=O)n(C)c2c1 ; *c1cc(N2CCCc3cc(-c4cnn(C)c4)c(C(F)F)cc32)c2cc(C)c(=O)n(C)c2c1 ; *c1cc(N2CCCc3cc(-c4cnn(C)c4)c(C#N)cc32)c2cc(C)c(=O)n(C)c2c1]

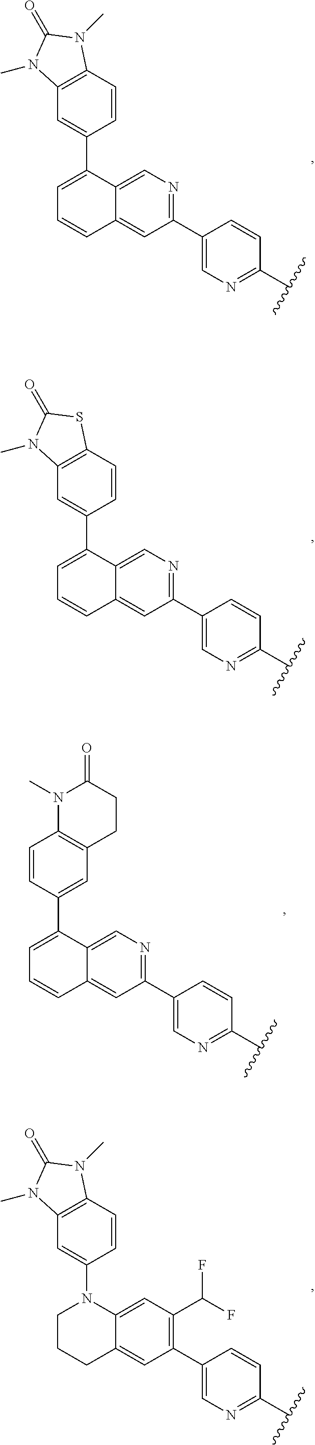 [SMILES: *c1ccc(-c2cc3cccc(-c4ccc5c(c4)n(C)c(=O)n5C)c3cn2)cn1 ; *c1ccc(-c2cc3cccc(-c4ccc5sc(=O)n(C)c5c4)c3cn2)cn1 ; *c1ccc(-c2cc3cccc(-c4ccc5c(c4)CCC(=O)N5C)c3cn2)cn1 ; *c1ccc(-c2cc3c(cc2C(F)F)N(c2ccc4c(c2)n(C)c(=O)n4C)CCC3)cn1]

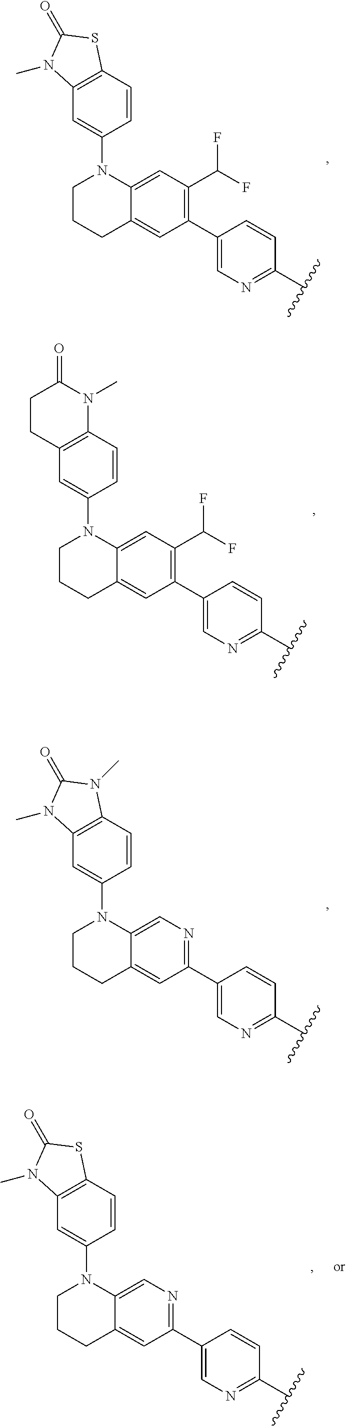 [SMILES: *c1ccc(-c2cc3c(cc2C(F)F)N(c2ccc4sc(=O)n(C)c4c2)CCC3)cn1 ; *c1ccc(-c2cc3c(cc2C(F)F)N(c2ccc4c(c2)CCC(=O)N4C)CCC3)cn1 ; *c1ccc(-c2cc3c(cn2)N(c2ccc4c(c2)n(C)c(=O)n4C)CCC3)cn1 ; *c1ccc(-c2cc3c(cn2)N(c2ccc4sc(=O)n(C)c4c2)CCC3)cn1]

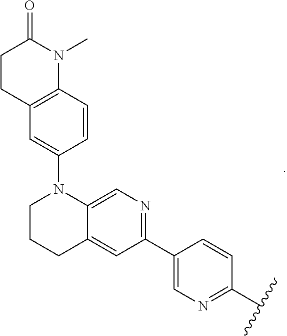 [SMILES: *c1ccc(-c2cc3c(cn2)N(c2ccc4c(c2)CCC(=O)N4C)CCC3)cn1]

In some embodiments, SB-CBP/p300 has one of the following structures:

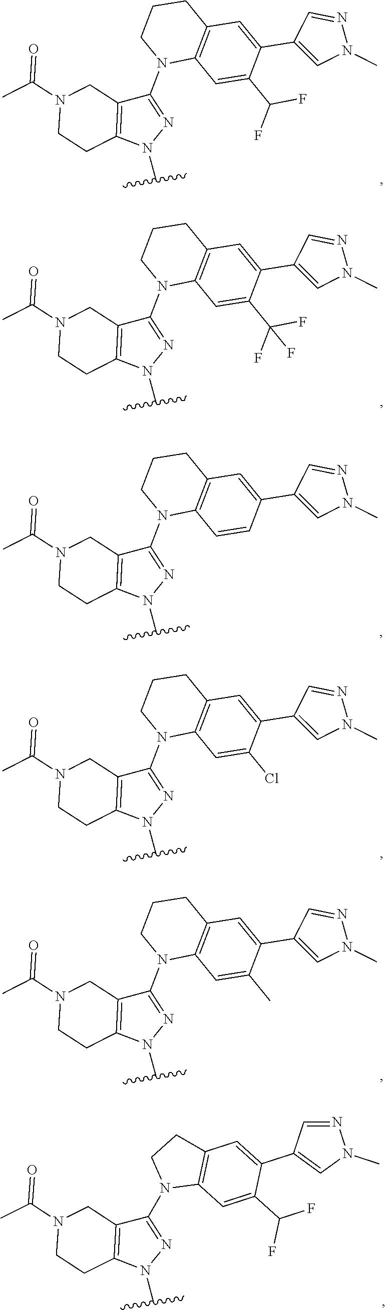

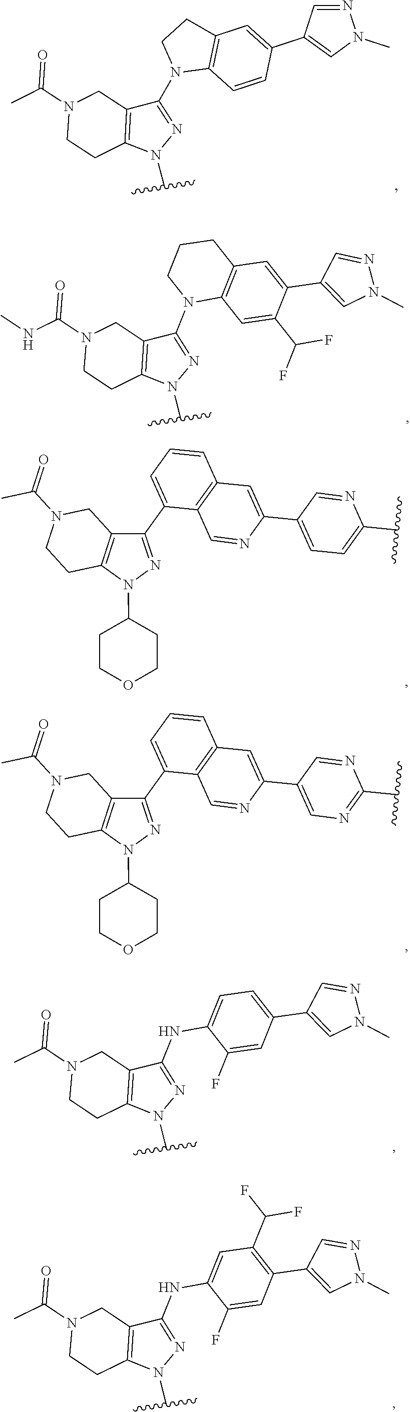

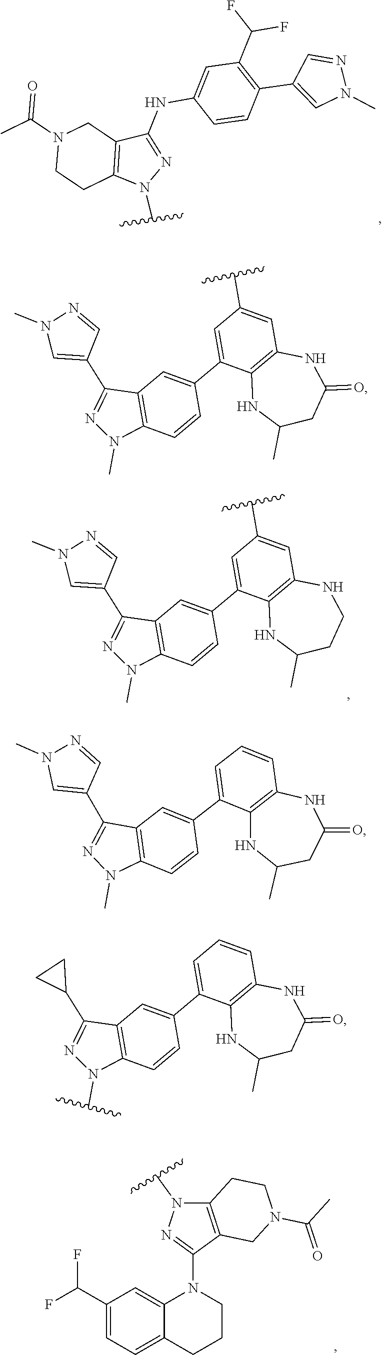

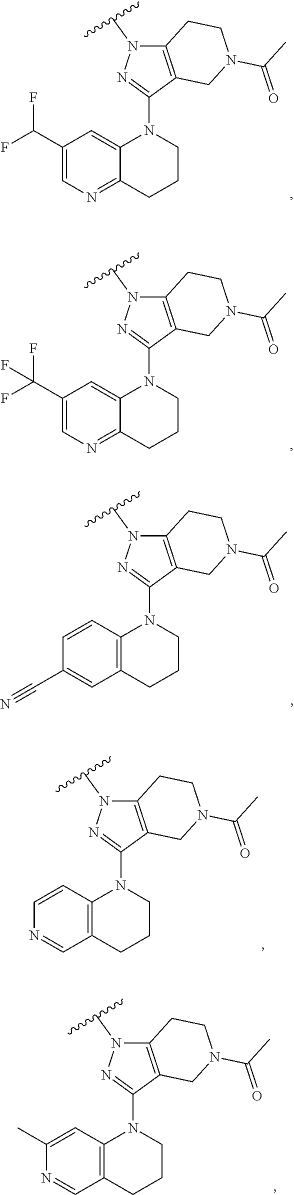

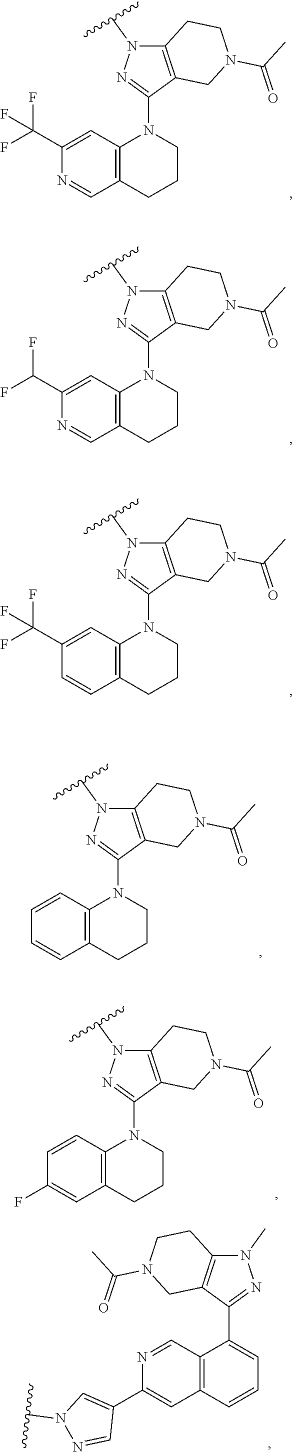

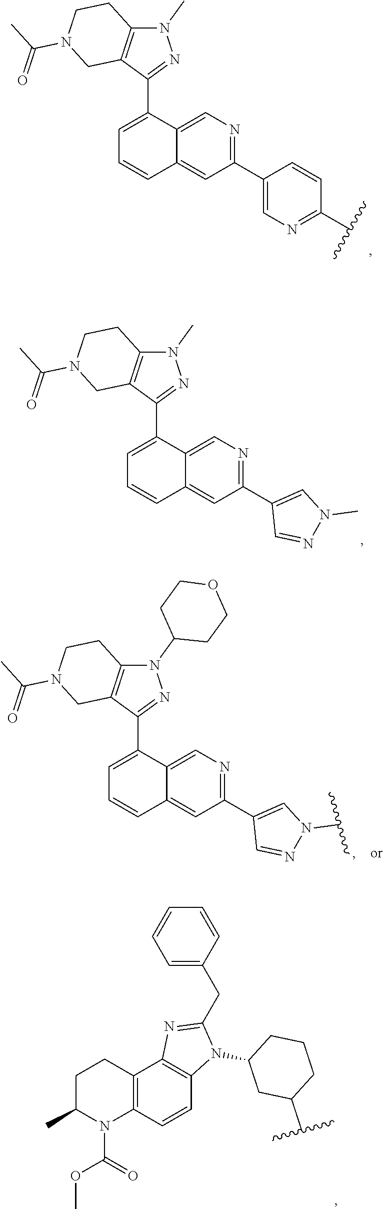

or a pharmaceutically acceptable salt or solvate thereof.

In some embodiments, SB-CBP/p300 has one of the following structures:

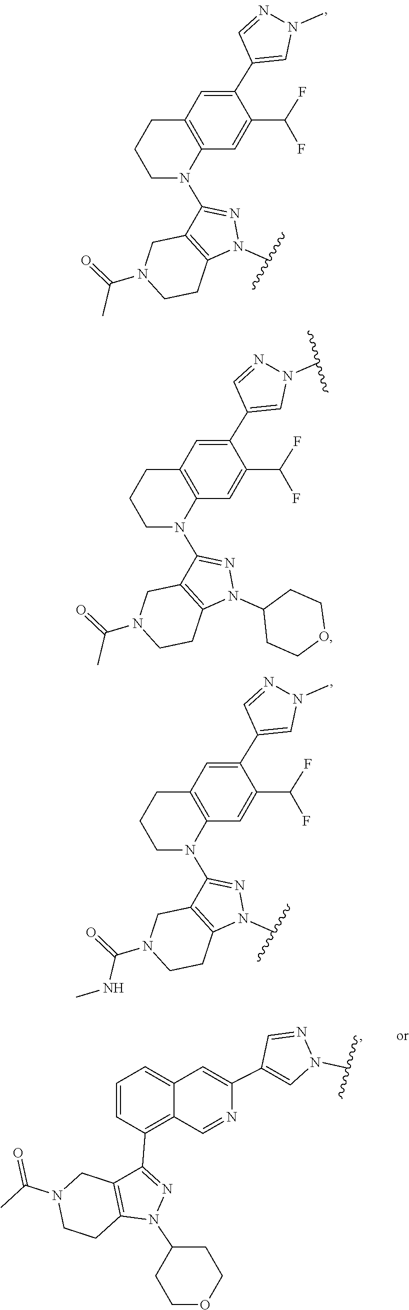

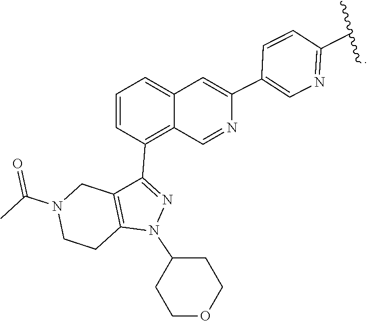 [SMILES: *c1ccc(-c2cc3cccc(-c4nn(C5CCOCC5)c5c4CN(C(C)=O)CC5)c3cn2)cn1]

In some embodiments, SB-CBP/p300 has one of the following structures:

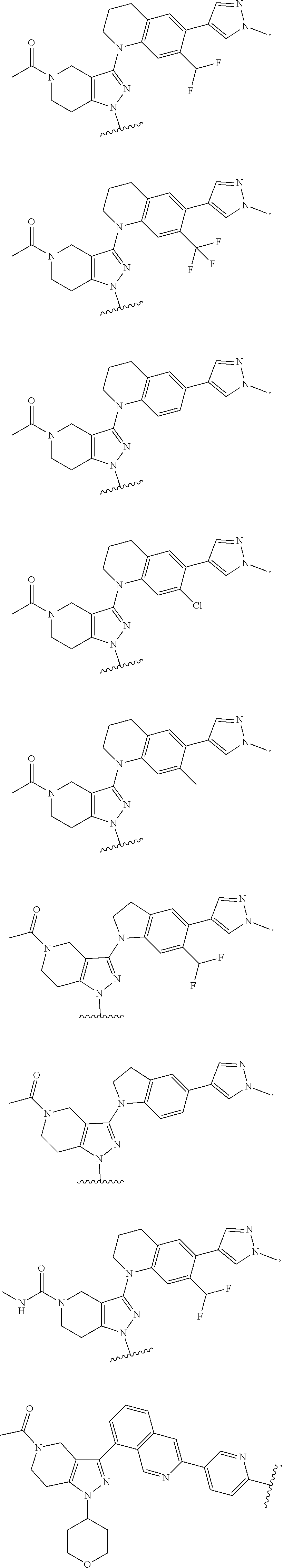

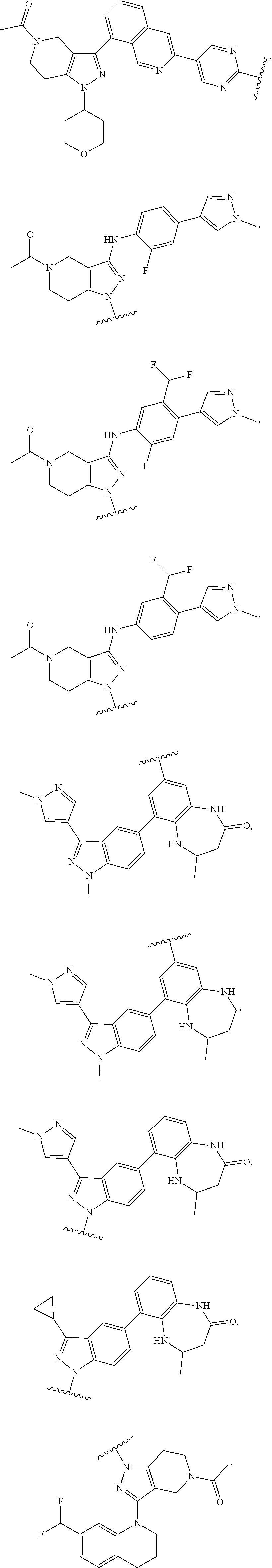

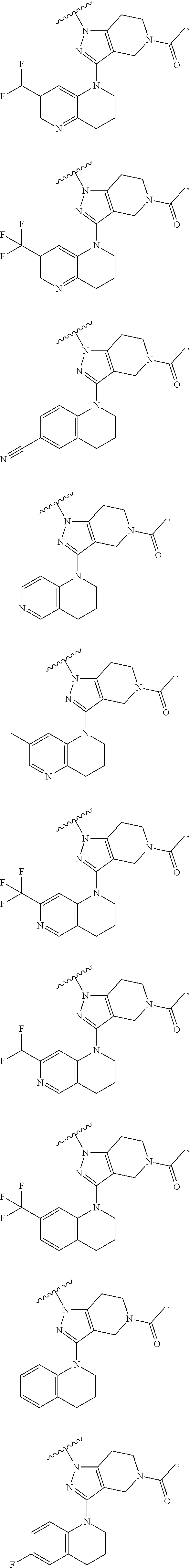

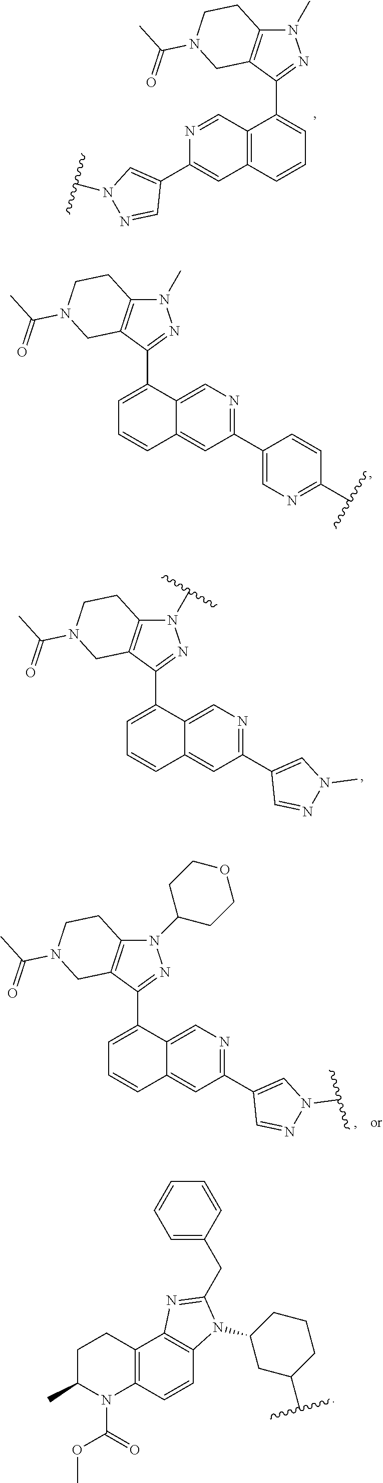

In some embodiments, R³² is

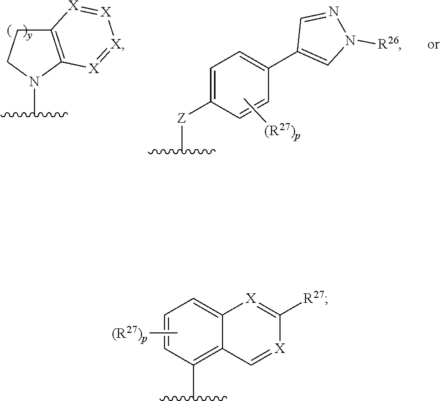

each R²⁷ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, substituted or unsubstituted heteroaryl, —CN, —OH, or —OR^{a}; and each R^{a} is independently substituted or unsubstituted C₁-C₆alkyl.

In some embodiments, R³² is

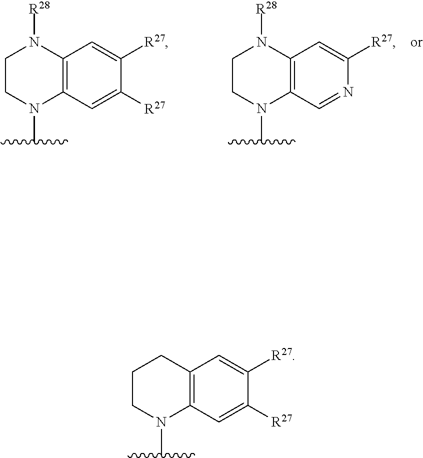

In some embodiments, each R²⁷ is independently hydrogen, —CH₃, —CH₂CH₃, —F, —CHF₂, —CF₃, —CN, —OH, —OCH₃, cyclopropyl, cyclobutyl, oxetanyl, tetrahydrofuranyl, tetrahydropyranyl, phenyl, pyrazolyl, 1-methyl pyrazolyl, pyridinyl, or pyrimidinyl.

In some embodiments, R³² is

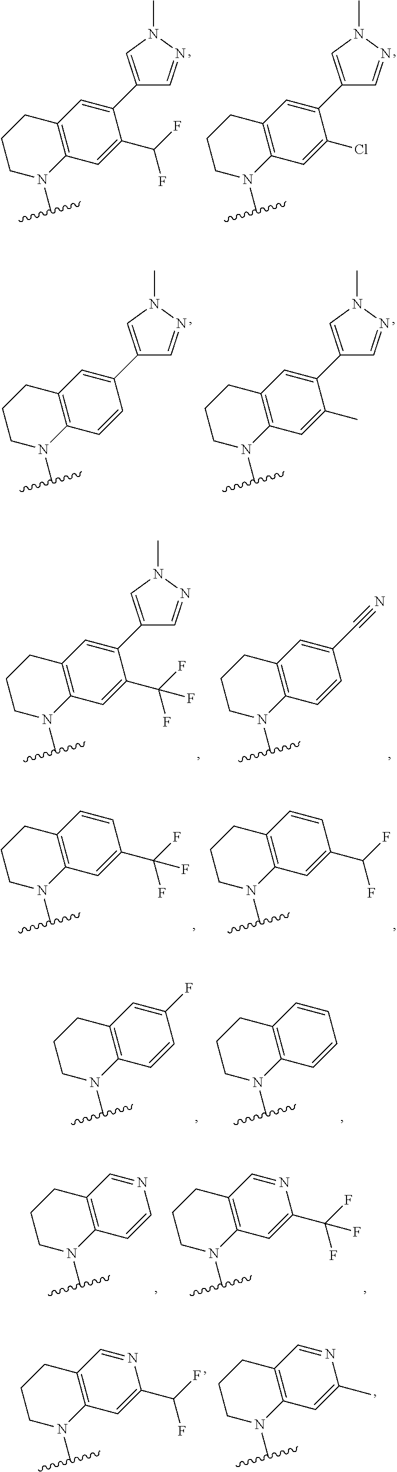

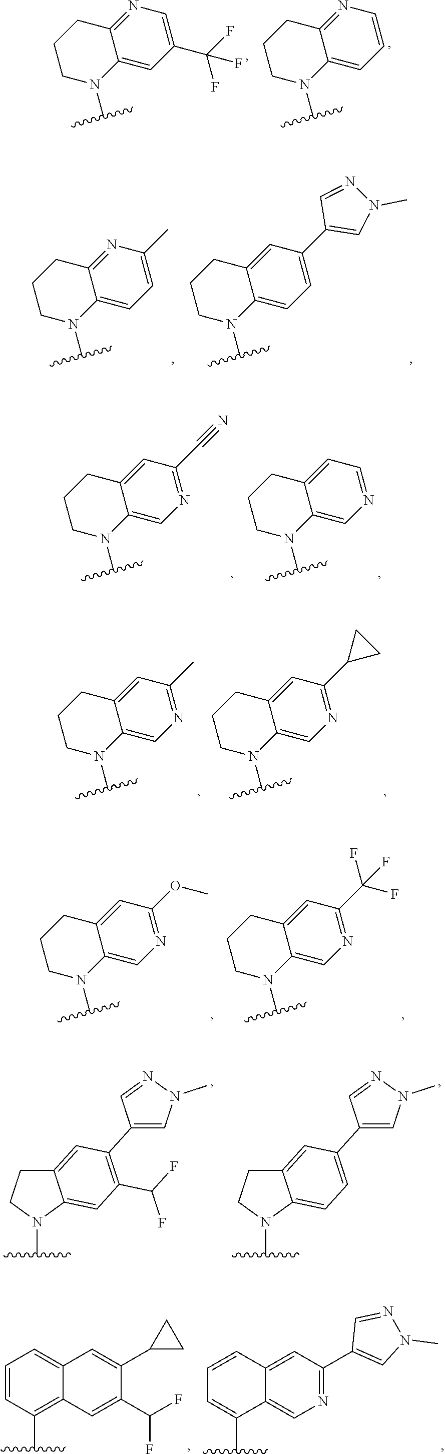

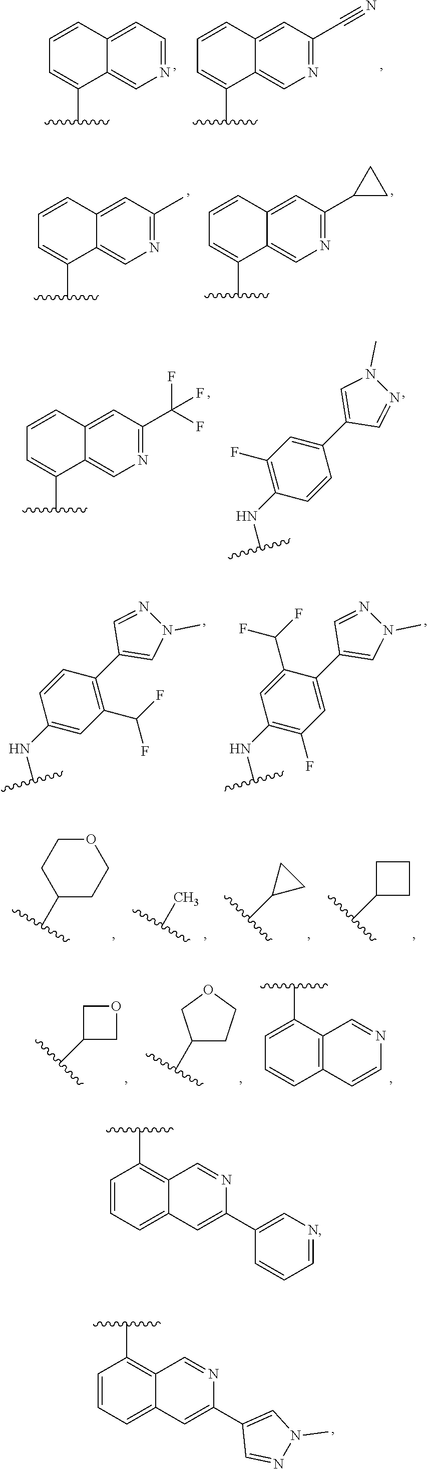

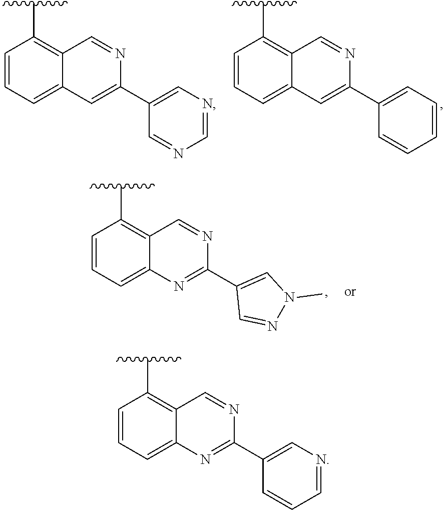

In some embodiments, R³² is

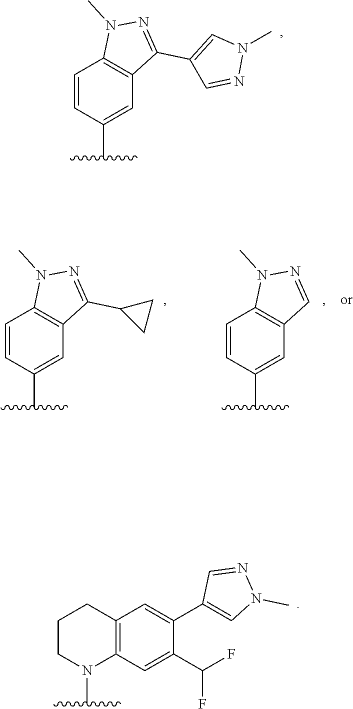 [SMILES: *c1ccc2c(c1)c(-c1cnn(C)c1)nn2C ; *c1ccc2c(c1)c(C1CC1)nn2C ; *c1ccc2c(cnn2C)c1 ; *N1CCCc2cc(-c3cnn(C)c3)c(C(F)F)cc21]

In some embodiments, R³² is

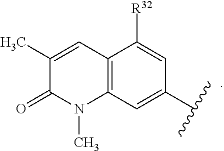

In some embodiments, R³² is

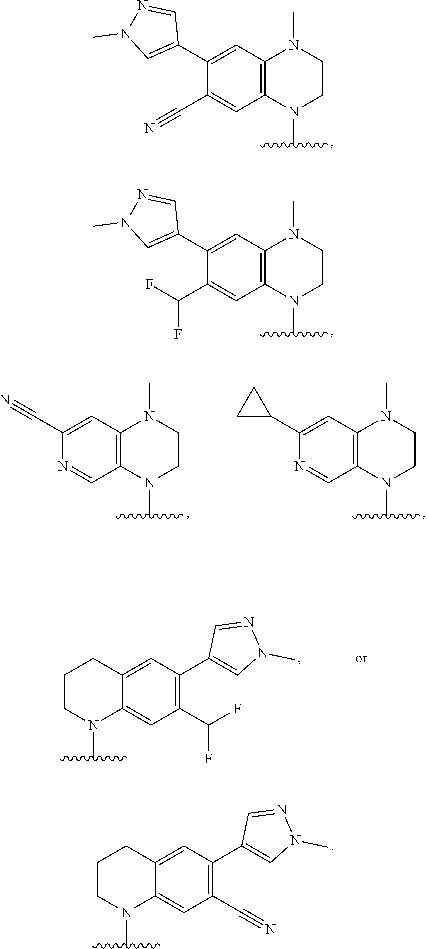 [SMILES: *N1CCN(C)c2cc(-c3cnn(C)c3)c(C#N)cc21 ; *N1CCN(C)c2cc(-c3cnn(C)c3)c(C(F)F)cc21 ; *N1CCN(C)c2cc(C#N)ncc21 ; *N1CCN(C)c2cc(C3CC3)ncc21 ; *N1CCCc2cc(-c3cnn(C)c3)c(C(F)F)cc21 ; *N1CCCc2cc(-c3cnn(C)c3)c(C#N)cc21]

In some embodiments, R³² is

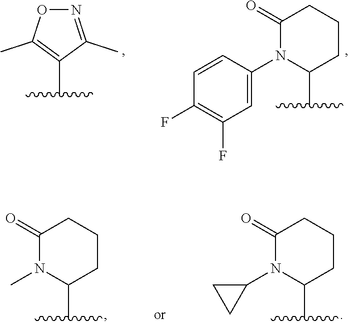

In some embodiments, R³² is

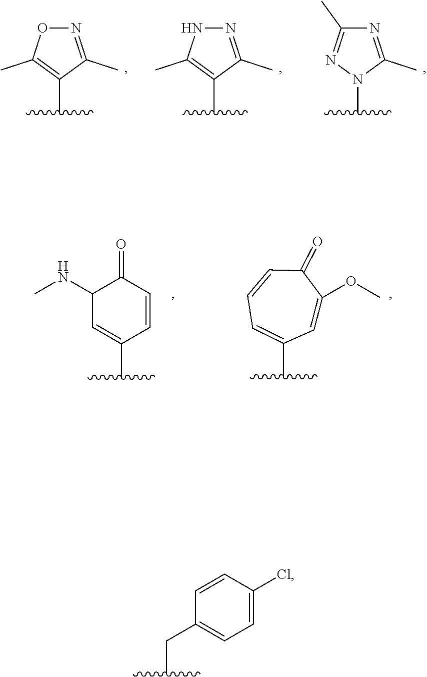

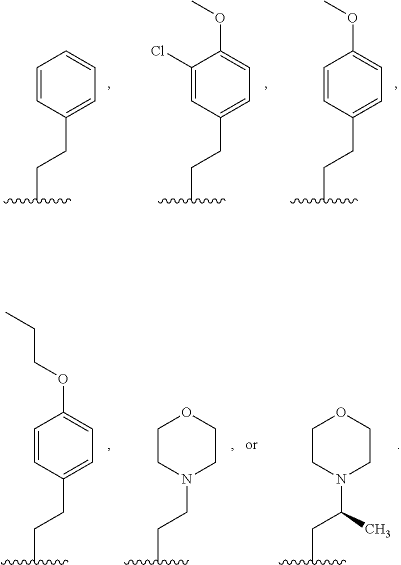

In some embodiments, the DP is a nuclear nuclear receptor (NR) transcription factor.

In some embodiments, the DP is a nuclear nuclear receptor (NR) transcription factor that is the androgen receptor (AR) and the BDP is a binder of AR (B-AR); or the DP is the estrogen receptor (ER) and the BDP is a binder of ER (B-ER); or retinoic acid receptor-related orphan nuclear receptor γ (RORγ) and the B-DP is a binder of RORγ (B-ROR).

In some embodiments, the DP is the androgen receptor (AR) and the BDP is a binder of AR (B-AR), wherein the B-AR is an AR antagonist, a selective androgen receptor modulator (SARM), or selective androgen receptor degrader (SARD); and the B-ER is an ER antagonist, a selective estrogen receptor modulator (SERM), or a selective estrogen receptor degrader (SERD); wherein the AR antagonist, SARM, or SARD is a non-steroidal AR ligand or a steroidal AR ligand; and wherein the ER antagonist, SERM, or SERD is a non-steroidal ER ligand or a steroidal ER ligand.

In some embodiments, the DP is the androgen receptor (AR) and the BDP is a binder of AR (B-AR) that is an AR antagonist, a selective androgen receptor modulator (SARM), or a selective androgen receptor degrader (SARD); wherein the AR antagonist, SARM, or SARD binds to the ligand-binding domain (LBD) of AR.

In some embodiments, the AR antagonist, SARM, or SARD comprises: a head moiety that occupies the LBD region of the AR; and 1) an optional core moiety; 2) an optional tail moiety covalently attached to the core moiety; or 3) both 1) and 2).

In some embodiments, the head moiety of the AR antagonist, SARM, or SARD forms hydrogen bonds with the side chains of Gln 711 and Arg752 of the LBD of AR.

In some embodiments, the optional linker is covalently attached to the head moiety if the optional core moiety and optional tail moiety are absent; or the optional linker is covalently attached to the core moiety if the optional tail moiety is absent; or the optional linker is covalently attached to the tail moiety.

In some embodiments, B-AR is a non-steroidal AR ligand or a steroidal AR ligand.

In some embodiments, B-AR is flutamide, hydroxylflutamide, nilutamide, bicalutamide, enzalutamide, apalutamide, ODM201, AZD3514, or BMS641988.

In some embodiments, B-AR comprises ahead group and an optional core, wherein the head group is selected from:

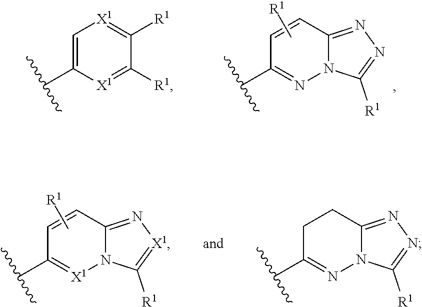

each X¹ is independently —CR¹— or —N—;
each R¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted 3- to 8-membered heterocycloalkyl, —CN, —NO₂, —OH, —OR⁴, OC(═O)R⁴, —OC(═O)N(R⁵)₂, —OC(═O)OR⁵, —OC(═O)NR⁵, —SH, —SR⁴, —S(═O)R⁴, —S(═O)₂R⁵, —S(═O)₂OR⁴, —S(═O)₂N(R⁵)₂, —N(R⁵)₂, —NR⁵C(═O)NR⁵, —NR⁵C(═O)R⁴, —NR⁵C(═O)OR⁵, —NR⁵S(═O)₂R⁵, —C(═O)R⁵, —C(═O)OR⁵, or —C(═O)N(R⁵)₂.
each R⁴ is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl; and each R⁵ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R⁵ on the same N atom are taken together with the N atom to which they are attached to form a substituted or unsubstituted N-containing heterocycle.

In some embodiments, the head group is selected from:

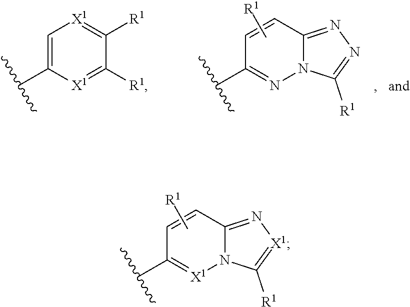

one X¹ is —CR¹— and the other X¹ is —CR¹— or —N—; each R¹ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, —OCH₂CH₃, —CN, —C(═O)NH₂ or —C(═O)NH(CH₃).

In some embodiments, the head group is selected from:

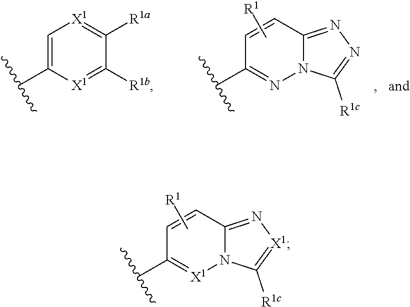

each X¹ is independently —CR¹— or —N—; R^{1a} is —CN, —NO₂, —C(═O)R⁵, —C(═O)OR⁵, or —C(═O)N(R⁵)₂; R^{1b} is hydrogen, halogen, C₁-C₄alkyl, C₁-C₄fluoroalkyl, —CN, —OH, —OR⁴, or —SR⁴; R^{1c} is hydrogen, halogen, C₁-C₄alkyl, C₁-C₄fluoroalkyl, or —CN; each R¹ is independently hydrogen, halogen, C₁-C₄alkyl, C₁-C₄fluoroalkyl, —CN, —OH, or —OR⁴; each R⁴ is independently C₁-C₄alkyl, or C₁-C₄fluoroalkyl; and each R⁵ is independently hydrogen, C₁-C₄alkyl, or C₁-C₄fluoroalkyl.

In some embodiments, the head group is selected from:

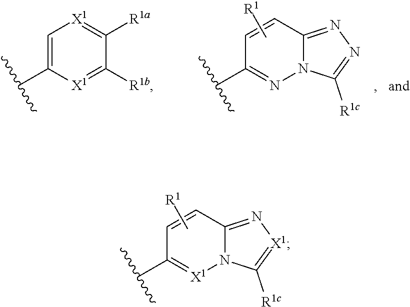

one X¹ is —CR¹— and the other X¹ is —CR¹— or —N—; R^{1a} is —CN, —NO₂, —C(═O)NH₂ or —C(═O)NH(CH₃); R^{1b} is hydrogen, F, Cl, Br, I, —CH₃, —CH₂CH₃, —CH₂F, —CHF₂, —CF₃, —OH, —OCF₃, —OCH₃, —OCH₂CH₃, or —CN; each R^{1c} is hydrogen, F, Cl, Br, —CH₃, —CH₂F, —CHF₂, or —CF₃; each R¹ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, or —OCH₂CH₃. In some embodiments, the head group:

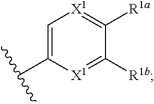

one X¹ is —CR¹— and the other X¹ is —CR¹— or —N—; R^{1a} is —CN; R^{1b} is hydrogen, F, Cl, —CH₃, —CH₂F, —CHF₂, —CF₃, —OCF₃, or —OCH₃; each R¹ is independently hydrogen, F, Cl, —CH₃, —CH₂F, —CHF₂, —CF₃, —OCF₃, or —OCH₃. In some embodiments, the head group:

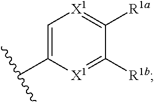

one X¹ is —CR¹— and the other X¹ is —CR¹—; R^{1a} is —CN; R^{1b} is Cl, —CH₃, —CF₃, or —OCH₃; each R¹ is independently hydrogen, or —CH₃.

In some embodiments, the head group is selected from:

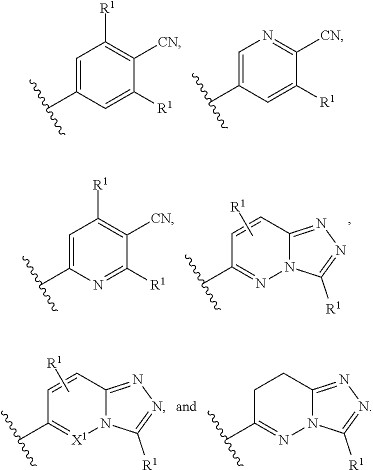

In some embodiments, the head group is selected from:

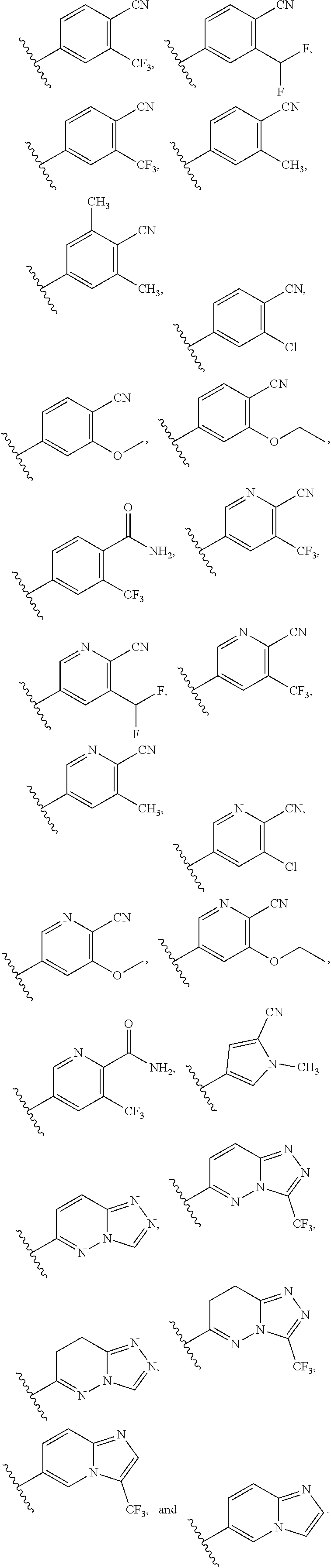

In some embodiments, the optional core comprises a group selected from:

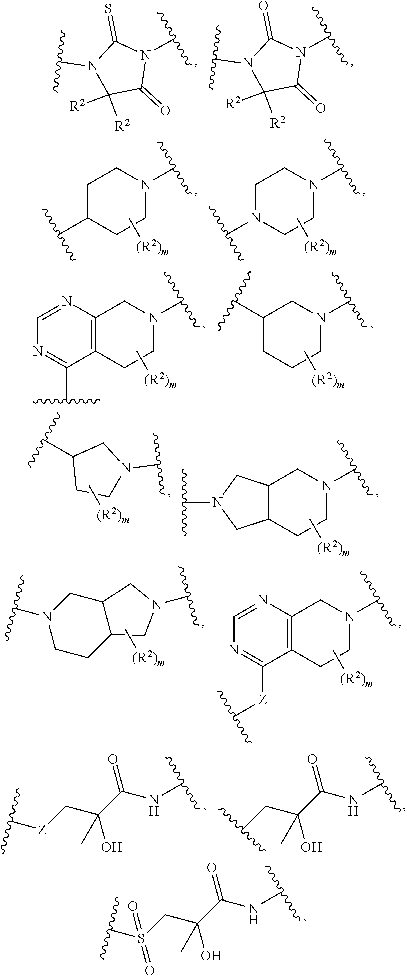

Z is —O— or —NR⁵—;

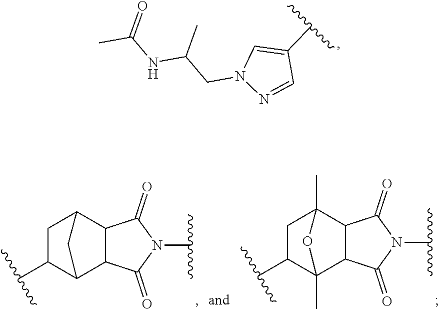 [SMILES: *c1cnn(CC(C)NC(C)=O)c1 ; *C1CC2CC1C1C(=O)N(*)C(=O)C21 ; *C1CC2(C)OC1(C)C1C(=O)N(*)C(=O)C12]

each R² is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR⁴, OC(═O)R⁴, —S(═O)R⁴, —S(═O)₂R⁵, —S(═O)₂N(R⁵)₂, —N(R⁵)₂, —NR⁵C(═O)NR⁵, —NR⁵C(═O)R⁴, —C(═O)R⁵, —C(═O)OR⁵, or —C(═O)N(R⁵)₂; or
two R² on the same carbon atom are taken together with the carbon atom to which they are attached to form a substituted or unsubstituted C₃-C₈cycloalkyl or a substituted or unsubstituted monocyclic 3- to 8-membered heterocycloalkyl;
m is 0, 1, 2, 3, or 4; each s is independently 1, 2, or 3;
each R⁴ is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R⁵ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl; or two R⁵ on the same N atom are taken together with the N atom to which they are attached to form a substituted or unsubstituted N-containing heterocycle.

In some embodiments, the optional core comprises a group selected from:

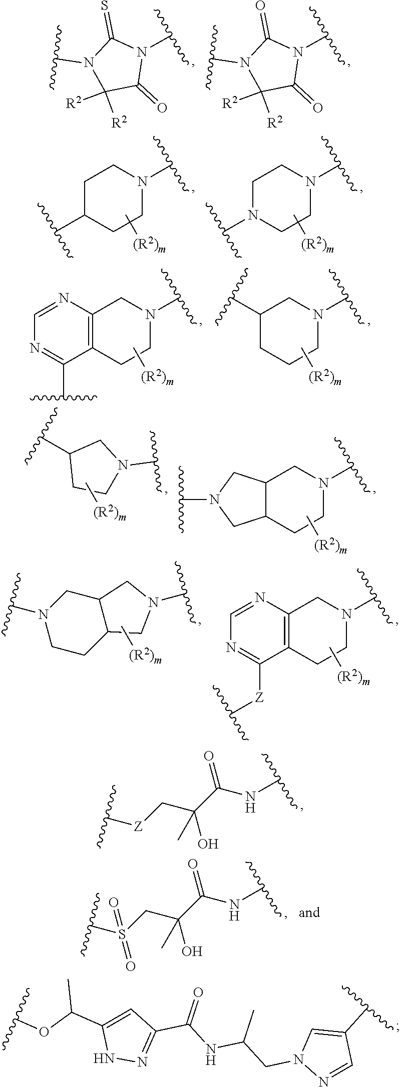

Z is —O— or —NR⁵—;
each R² is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR⁴, OC(═O)R⁴, —S(═O)R⁴, —S(═O)₂R⁵, —S(═O)₂N(R⁵)₂, —N(R⁵)₂, —NR⁵C(═O)NR⁵, —NR⁵C(═O)R⁴, —C(═O)R⁵, —C(═O)OR⁵, or —C(═O)N(R⁵)₂; or
two R² on the same carbon atom are taken together with the carbon atom to which they are attached to form a substituted or unsubstituted C₃-C₈cycloalkyl or a substituted or unsubstituted monocyclic 3- to 8-membered heterocycloalkyl;
m is 0, 1, 2, 3, or 4; each s is independently 1, 2, or 3;
each R⁴ is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R⁵ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R⁵ on the same N atom are taken together with the N atom to which they are attached to form a substituted or unsubstituted N-containing heterocycle.

In some embodiments, the optional core further comprises a ring D that is a 5-, 6-, 8-, 9- or 10-membered aryl or a 5-, 6-, 8-, 9- or 10-membered heteroaryl that is optionally substituted with s R³; each R³ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂. In some embodiments, the optional core further comprises a ring D that is a 6-membered phenyl or a 5-, or 6-membered heteroaryl that is optionally substituted with s R³; each R³ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂. In some embodiments, each R³ is independently hydrogen, halogen, C₁-C₄alkyl, C₁-C₄fluoroalkyl, —CN, —OH, —OR⁴, or —N(R⁵)₂. In some embodiments, each R³ is independently hydrogen, halogen, C₁-C₄alkyl, C₁-C₄fluoroalkyl, —CN, —OH, or —OR⁴.

In some embodiments, the optional core and optional tail comprises a group selected from:

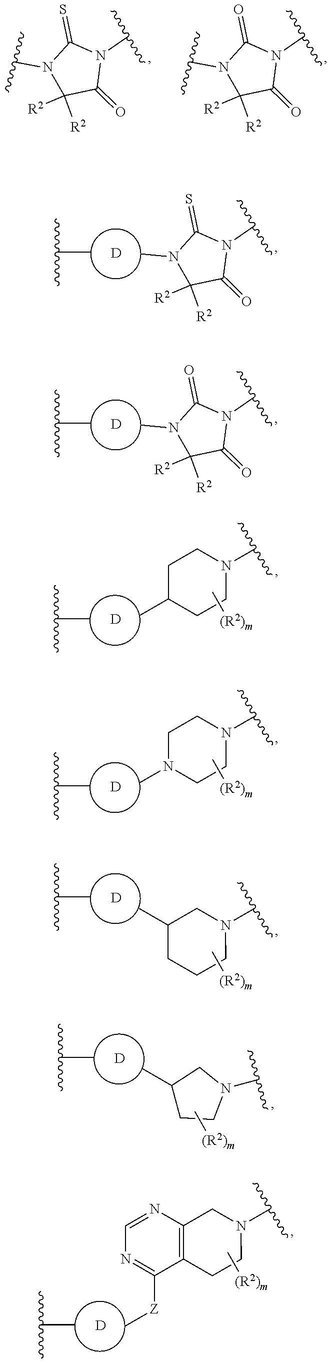

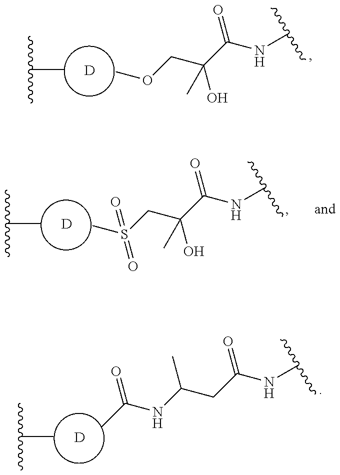

In some embodiments, the optional core comprises a group selected from:

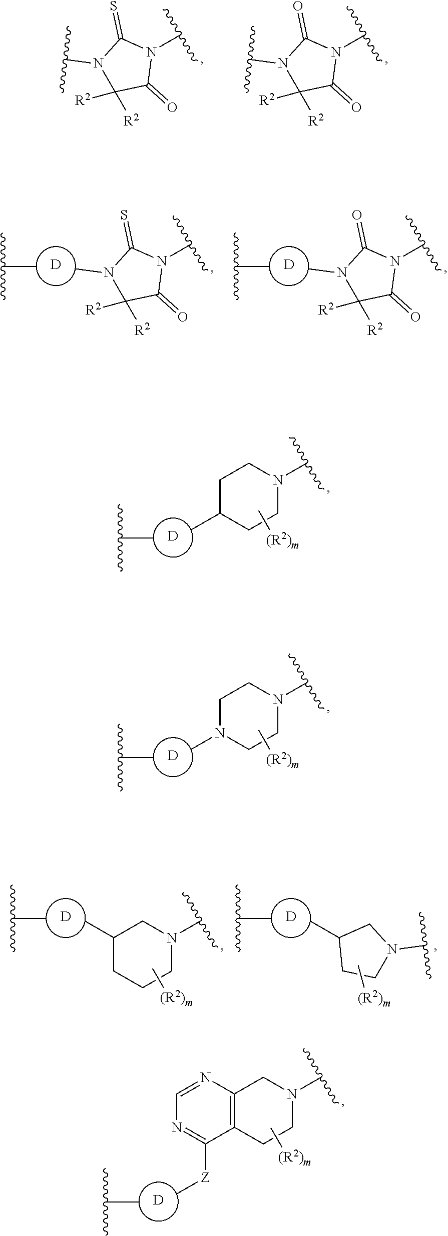

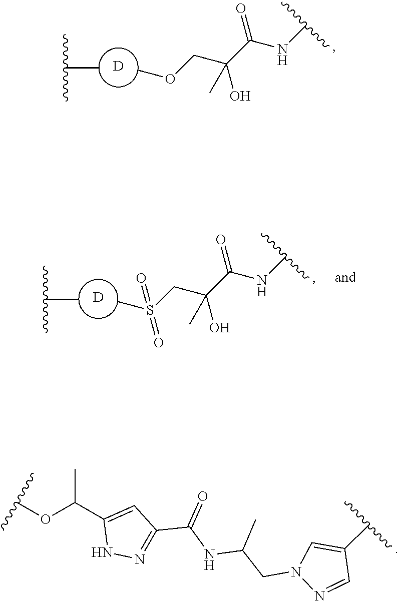

In some embodiments, ring D is phenyl, naphthyl, pyridinyl, pyrimidinyl, pyridazinyl, pyrazinyl, triazinyl, pyrrolyl, thiazolyl, oxazolyl, isoxazolyl, oxadiazolyl, imidazolyl, pyrazolyl, isothiazolyl, triazolyl, or tetrazolyl, wherein each ring D is optionally substituted substituted with s R³. In some embodiments, ring D is phenyl, wherein each ring D is optionally substituted substituted with s R³. In some embodiments, ring D is phenyl, pyridinyl, pyrimidinyl, pyridazinyl, or pyrazinyl, wherein each ring D is optionally substituted substituted with s R³. In some embodiments, ring D is pyridinyl, pyrimidinyl, pyridazinyl, or pyrazinyl, wherein each ring D is optionally substituted substituted with s R³. In some embodiments, ring D is pyrrolyl, thiazolyl, oxazolyl, isoxazolyl, oxadiazolyl, imidazolyl, pyrazolyl, isothiazolyl, triazolyl, or tetrazolyl, wherein each ring D is optionally substituted substituted with s R³. In some embodiments, ring D is pyrrolyl, pyrazolyl, or triazolyl, wherein each ring D is optionally substituted substituted with s R³.

In some embodiments, ring D is

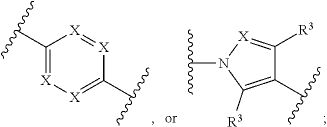

each X is independently —CR³— or —N—.

In some embodiments, ring D is

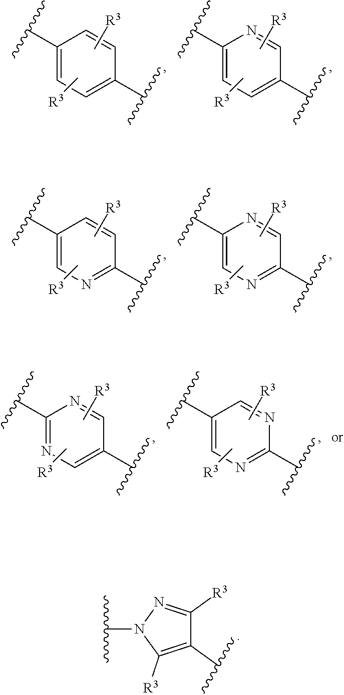

In some embodiments, m is 4 and each R² is substituted or unsubstituted C₁-C₆alkyl. In some embodiments, m is 4 and each R² is —CH₃. In some embodiments, two R² on the same carbon atom are taken together with the carbon atom to which they are attached to form a substituted or unsubstituted cyclobutyl, substituted or unsubstituted cyclopentyl, substituted or unsubstituted cyclohexyl, substituted or unsubstituted oxetanyl, substituted or unsubstituted tetrahydrofuranyl, substituted or unsubstituted tetrahydropyranyl, substituted or unsubstituted azetidine, substituted or unsubstituted pyrrolidinyl, or a substituted or unsubstituted piperidinyl. In some embodiments, two R² on the same carbon atom are taken together with the carbon atom to which they are attached to form a substituted or unsubstituted cyclobutyl.

In some embodiments, n is 2 and one R¹ is halogen and the other R¹ is —CN. In some embodiments, n is 2 and one R¹ is F, Cl, methyl, methoxy, or trifluoromethyl, and the other R¹ is —CN. In some embodiments, n is 2 and one R¹ is —Cl and the other R¹ is —CN. In some embodiments, n is 3 and one R¹ is Cl, one R¹ is methyl and the other R¹ is —CN. In some embodiments, n is 3 and one R¹ is methyl, one R¹ is methyl and the other R¹ is —CN. In some embodiments, n is 2 and one R¹ is F, Cl, methyl, methoxy, or trifluoromethyl, and the other R¹ is —CN. In some embodiments, n is 2 and one R¹ is —Cl and the other R¹ is —CN.

In some embodiments,

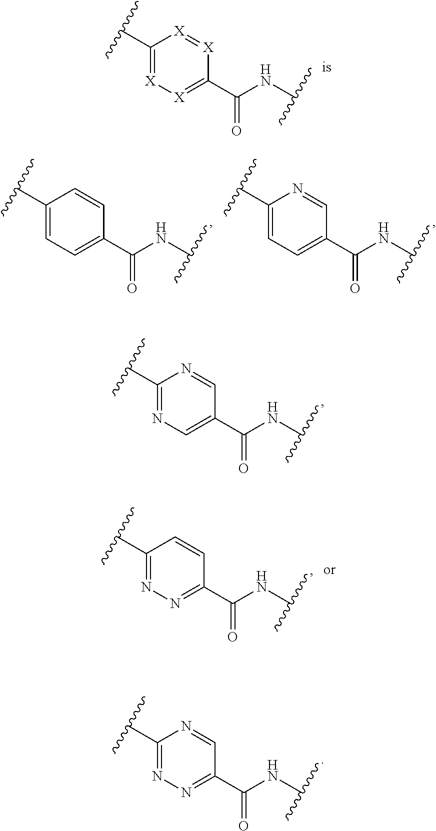

In some embodiments, B-AR has one of the following structures:

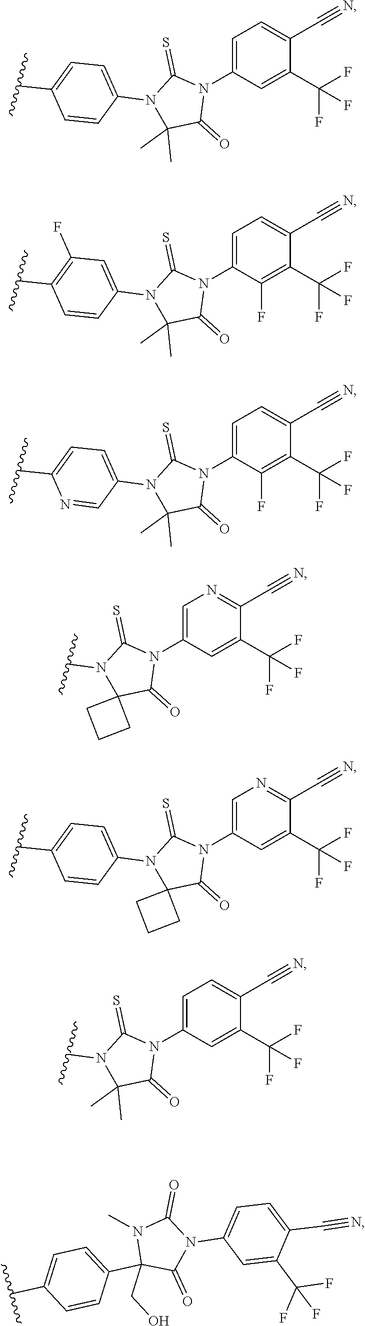

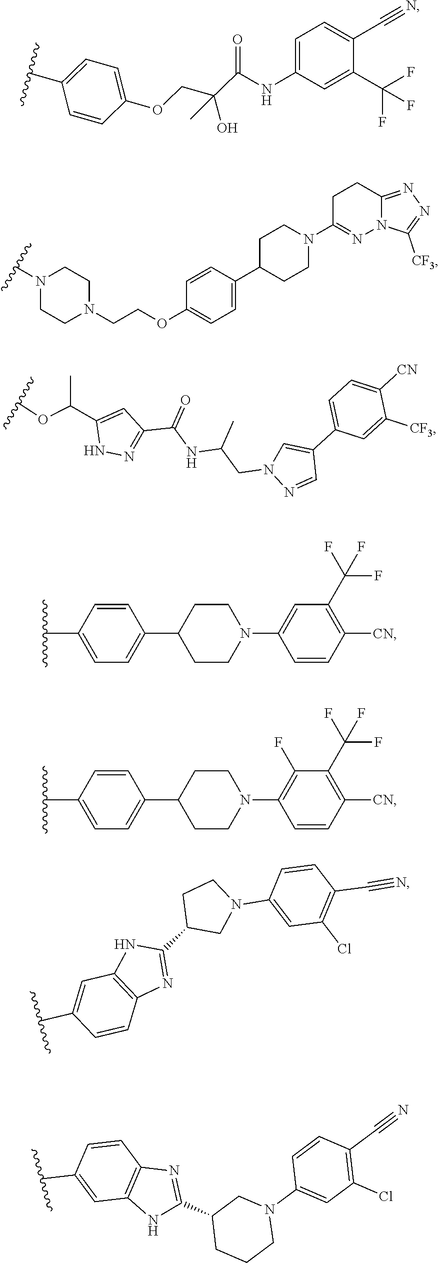

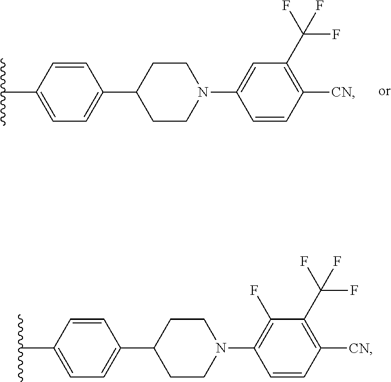 [SMILES: *c1ccc(C2CCN(c3ccc(C#N)c(C(F)(F)F)c3)CC2)cc1 ; "or" ; *c1ccc(C2CCN(c3ccc(C#N)c(C(F)(F)F)c3F)CC2)cc1]

or a pharmaceutically acceptable salt or solvate thereof.

In some embodiments, B—Ar has one of the following structures:

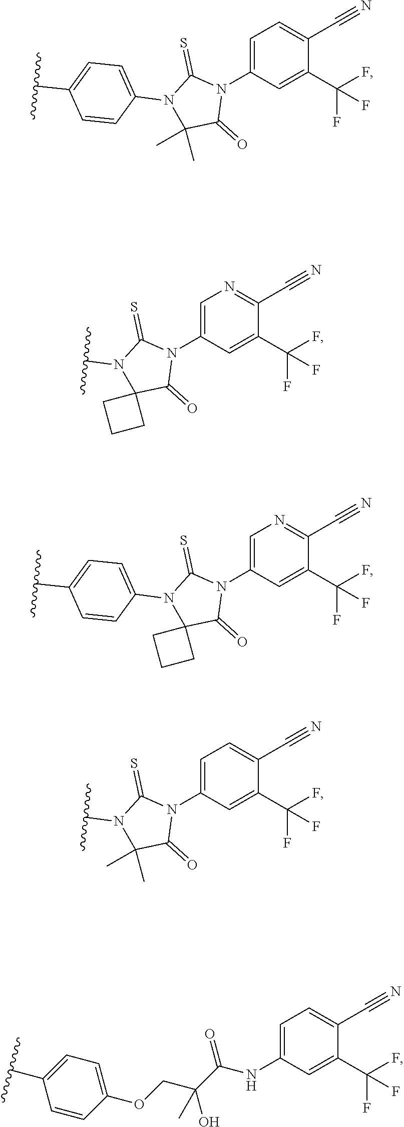 [SMILES: *c1ccc(N2C(=S)N(c3ccc(C#N)c(C(F)(F)F)c3)C(=O)C2(C)C)cc1 ; *N1C(=S)N(c2cnc(C#N)c(C(F)(F)F)c2)C(=O)C12CCC2 ; *c1ccc(N2C(=S)N(c3cnc(C#N)c(C(F)(F)F)c3)C(=O)C23CCC3)cc1 ; *N1C(=S)N(c2ccc(C#N)c(C(F)(F)F)c2)C(=O)C1(C)C ; *c1ccc(OCC(C)(O)C(=O)Nc2ccc(C#N)c(C(F)(F)F)c2)cc1]

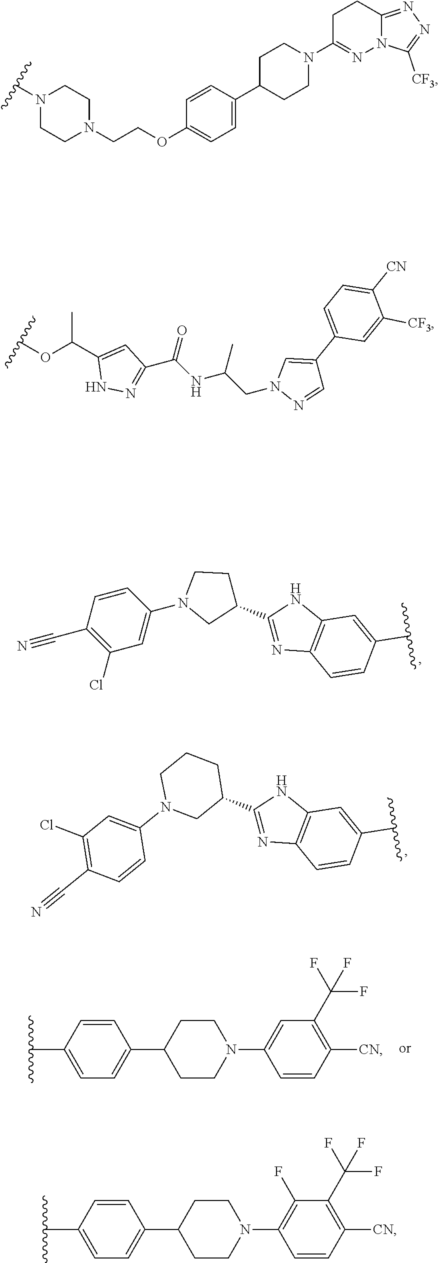

or a pharmaceutically acceptable salt or solvate thereof.

In some embodiments, the BDP is an AR antagonist. In some embodiments, the BDP is an AR antagonist that is flutamide, hydroxylflutamide, nilutamide, bicalutamide, enzalutamide, apalutamide, ODM201, AZD3514, or BMS641988.

In some embodiments, the BDP has one of the following structures:

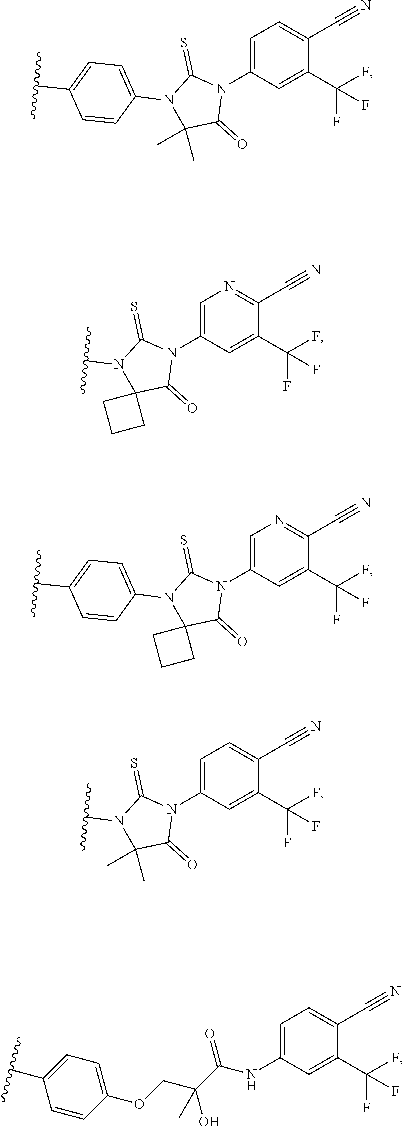 [SMILES: *c1ccc(N2C(=S)N(c3ccc(C#N)c(C(F)(F)F)c3)C(=O)C2(C)C)cc1 ; *N1C(=S)N(c2cnc(C#N)c(C(F)(F)F)c2)C(=O)C12CCC2 ; *c1ccc(N2C(=S)N(c3cnc(C#N)c(C(F)(F)F)c3)C(=O)C23CCC3)cc1 ; *N1C(=S)N(c2ccc(C#N)c(C(F)(F)F)c2)C(=O)C1(C)C ; *c1ccc(OCC(C)(O)C(=O)Nc2ccc(C#N)c(C(F)(F)F)c2)cc1]

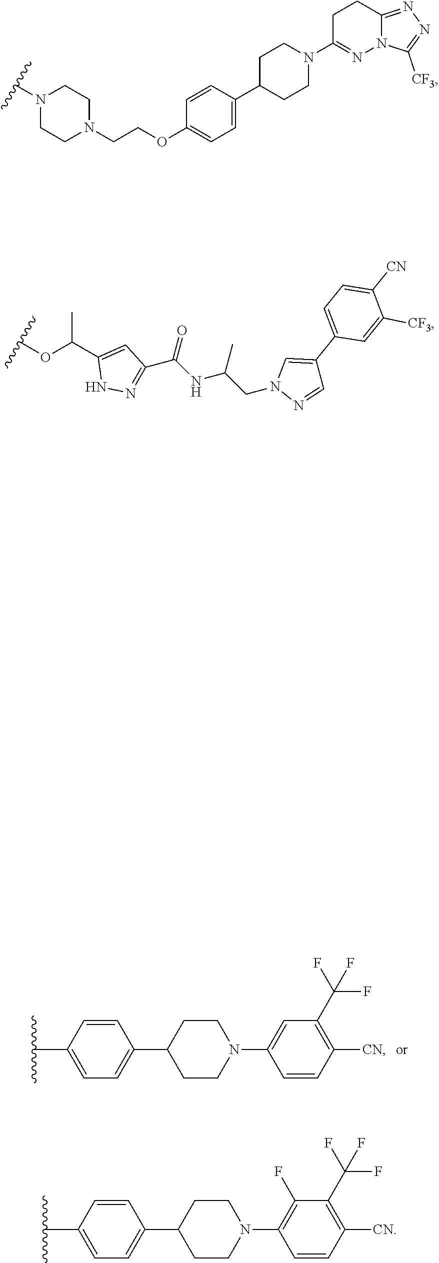 [SMILES: *N1CCN(CCOc2ccc(C3CCN(C4=Nn5c(nnc5C(F)(F)F)CC4)CC3)cc2)CC1 ; *OC(C)c1cc(C(=O)NC(C)Cn2cc(-c3ccc(C#N)c(C(F)(F)F)c3)cn2)n[nH]1 ; *c1ccc(C2CCN(c3ccc(C#N)c(C(F)(F)F)c3)CC2)cc1 ; "or" ; *c1ccc(C2CCN(c3ccc(C#N)c(C(F)(F)F)c3F)CC2)cc1]

In some embodiments, the BDP has one of the following structures:

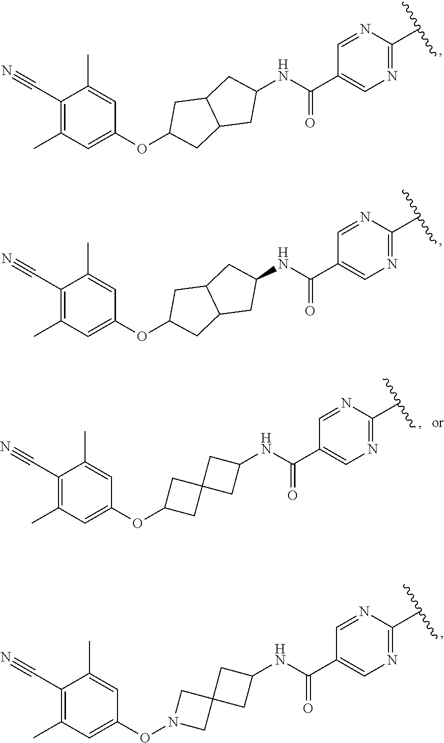

or a pharmaceutically acceptable salt or solvate thereof.

In some embodiments, the DP is the estrogen receptor (ER) and the BDP is a binder of ER (B-ER) that is an ER antagonist, a selective estrogen receptor modulator (SERM), or a selective estrogen receptor degrader (SERD). In some embodiments, the B-ER binds to the ligand-binding domain (LBD) of ER.

In some embodiments, the B-ER is:

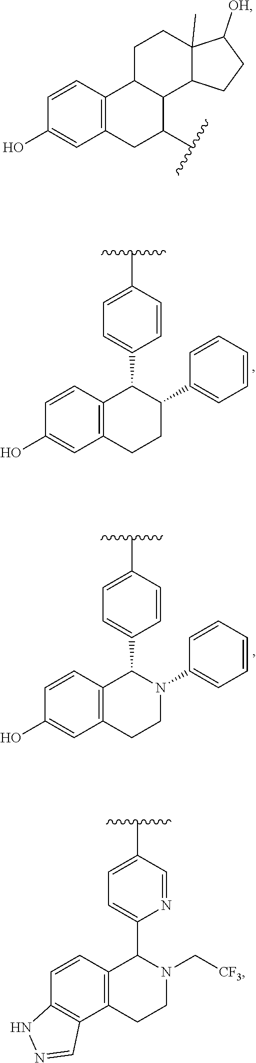

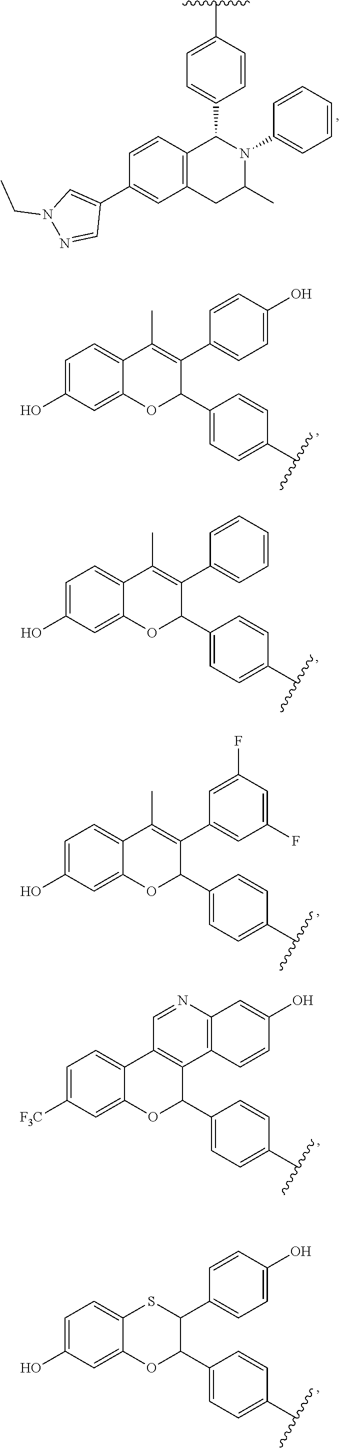

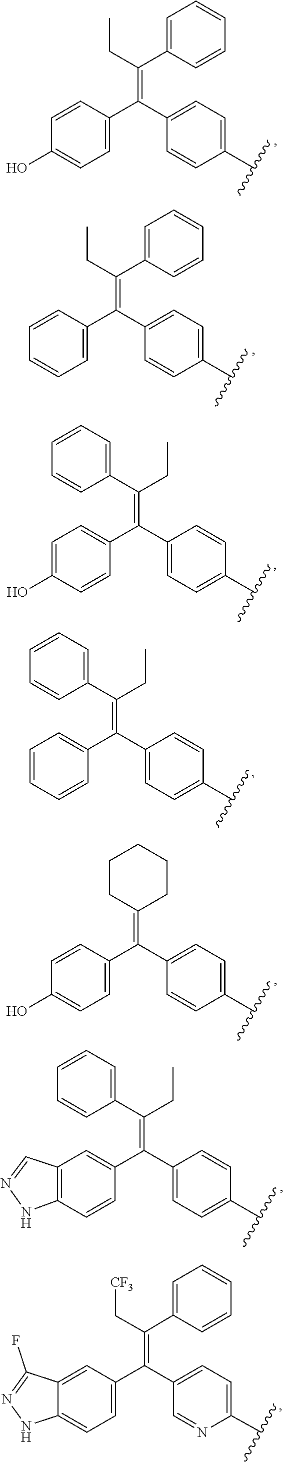

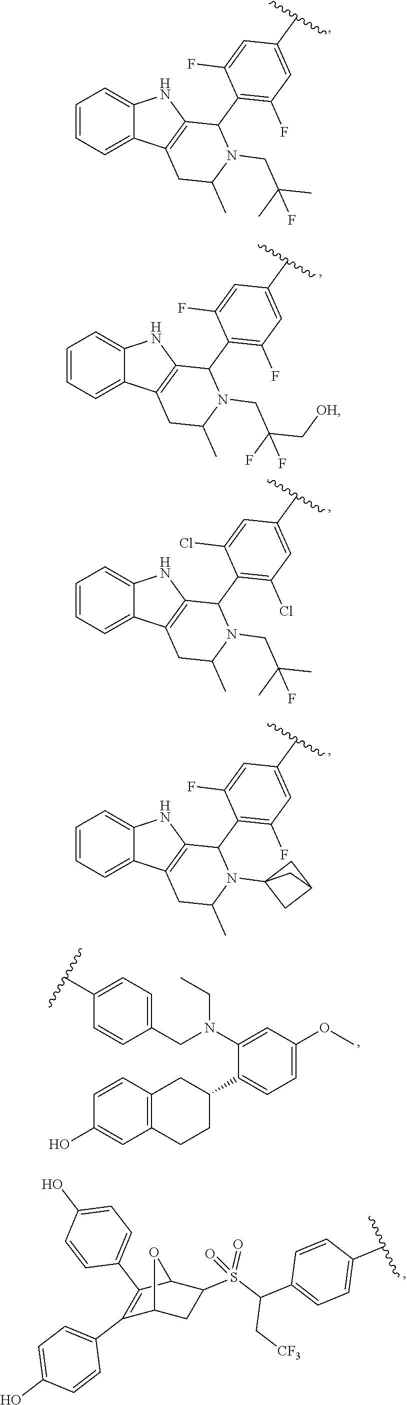

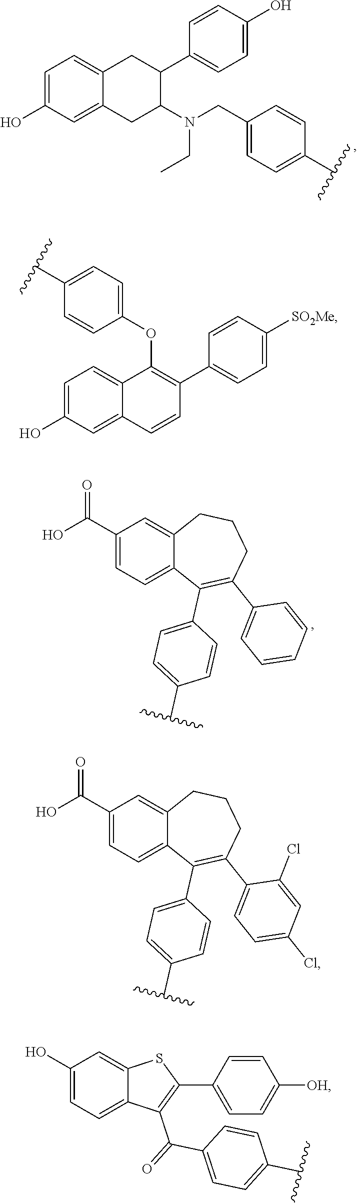 [SMILES: *c1ccc(CN(CC)C2Cc3cc(O)ccc3CC2c2ccc(O)cc2)cc1 ; *c1ccc(Oc2c(-c3ccc(S(C)(=O)=O)cc3)ccc3cc(O)ccc23)cc1 ; *c1ccc(C2=C(c3ccccc3)CCCc3cc(C(=O)O)ccc32)cc1 ; *c1ccc(C2=C(c3ccc(Cl)cc3Cl)CCCc3cc(C(=O)O)ccc32)cc1 ; *c1ccc(C(=O)c2c(-c3ccc(O)cc3)sc3cc(O)ccc23)cc1]

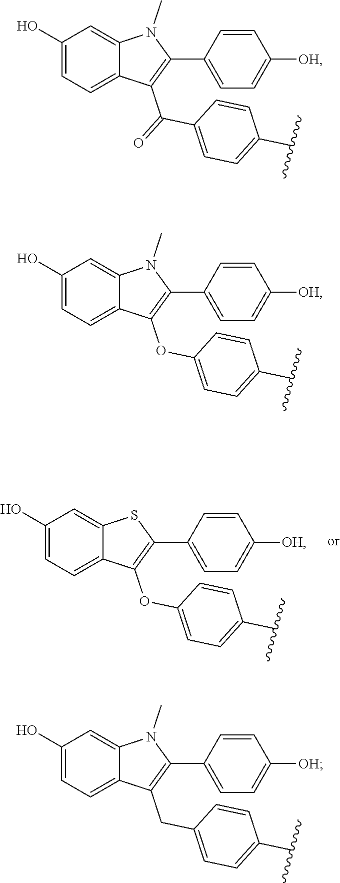 [SMILES: *c1ccc(C(=O)c2c(-c3ccc(O)cc3)n(C)c3cc(O)ccc23)cc1 ; *c1ccc(Oc2c(-c3ccc(O)cc3)n(C)c3cc(O)ccc23)cc1 ; *c1ccc(Oc2c(-c3ccc(O)cc3)sc3cc(O)ccc23)cc1 ; "or" ; *c1ccc(Cc2c(-c3ccc(O)cc3)n(C)c3cc(O)ccc23)cc1]

or a pharmaceutically acceptable salt or solvate thereof.

In some embodiments, the DP is chymase and the B-DP is a chymase inhibitor (e.g., see Taylor S J, et al., Discovery of potent, selective chymase inhibitors via fragment linking strategies. J Med Chem. 2013 Jun. 13; 56(11):4465-81). In some embodiments, the chymase inhibitor is selective for chymase over cathepsin G. In some embodiments, the chymase inhibitor binds to Ser195 in the active cleft of chymase, binds to the S1 pocket of chymase, or both.

In some embodiments, the chymase inhibitor is HY-109059, Fulacimstat, BAY1142524, HY-12370, TY-51469, HY-100269, Chymase-IN-1, HY-12514, NK3201, NK3201, HY-122161, JNJ-10311795, RWJ-355871, JNJ-10311795 (RWJ-355871), HY-126988, TPC-806, SP-Chymostatin B, or analog thereof.

In some embodiments, the chymase inhibitor has one of the following structures:

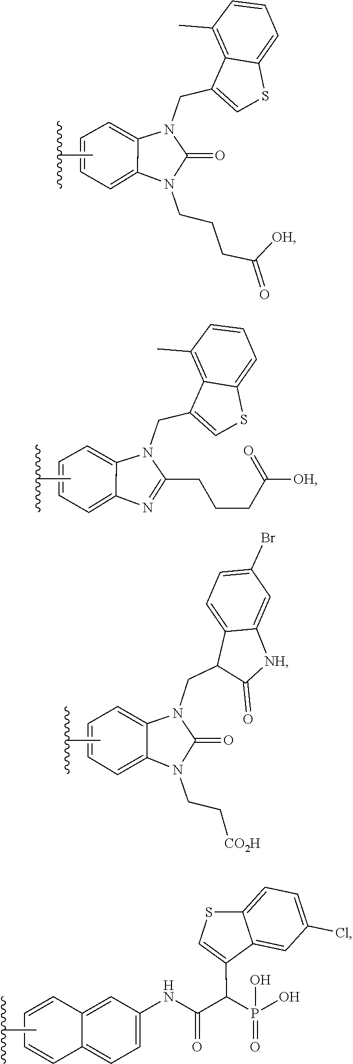

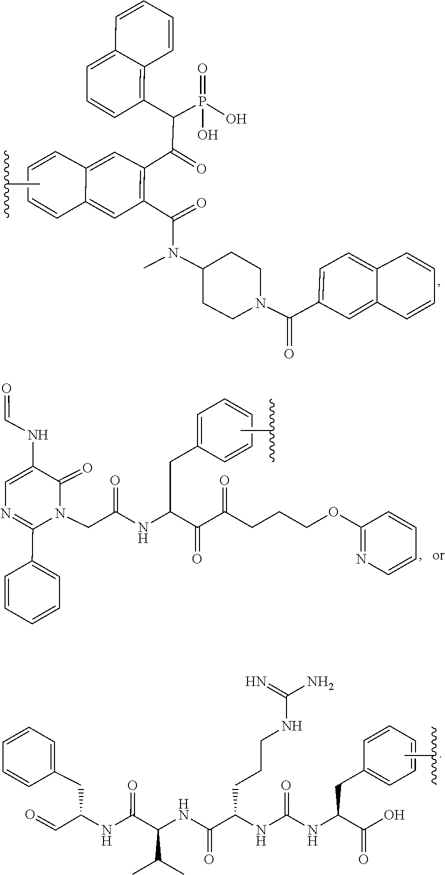

In some embodiments, the DP is retinoic acid receptor-related orphan nuclear receptor γ (RORγ) and the B-DP is a binder of RORγ (B-ROR) (e.g., see Christian Harcken, et al., Discovery of a Series of Pyrazinone RORγ Antagonists and Identification of the Clinical Candidate BI 730357; ACS Medicinal Chemistry Letters 2021 12 (1), 143-154; Jiuping Zeng, et al., Small molecule inhibitors of RORγt for Th17 regulation in inflammatory and autoimmune diseases, Journal of Pharmaceutical Analysis, Volume 13, Issue 6, 2023, Pages 545-562). In some embodiments, the B-ROR is a RORγ antagonist, RORγ inverse agonist, RORγ inhibitor, or RORγ agonist. In some embodiments, the B-ROR binds to the ligand-binding domain (LBD) of RORγ. In some embodiments, the B-ROR is S18-000003, A213, SHR168442, TMP778, BMS-986251, BMS-986313, A-9758, Digoxin, FC99, JNJ-61803534, SR2211, Ursolic acid, SR1001, TAK-828F, JNJ-61803534, JTE-451 (Retezorogant), BI 730357 (Bevurogant), ABBV-157 (Cedirogant), RTA-1701, AUR101, GSK2981278, PF-06763809, VTP-43742 (Vimirogant), AZD0284, GSK805, VPR-254, BI119, CQMU152, BIX119, VTP-938, or analog thereof.

In some embodiments, the B-ROR has one of the following structures:

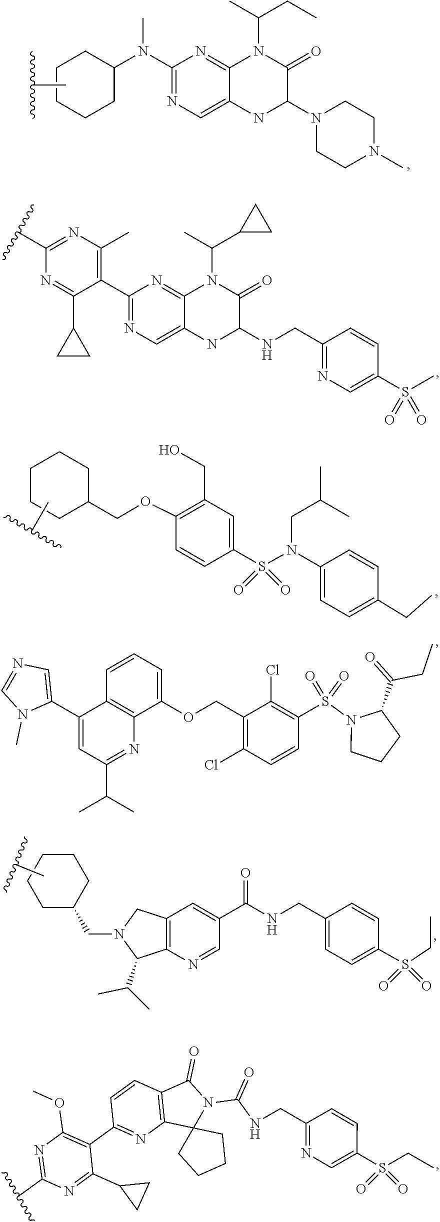

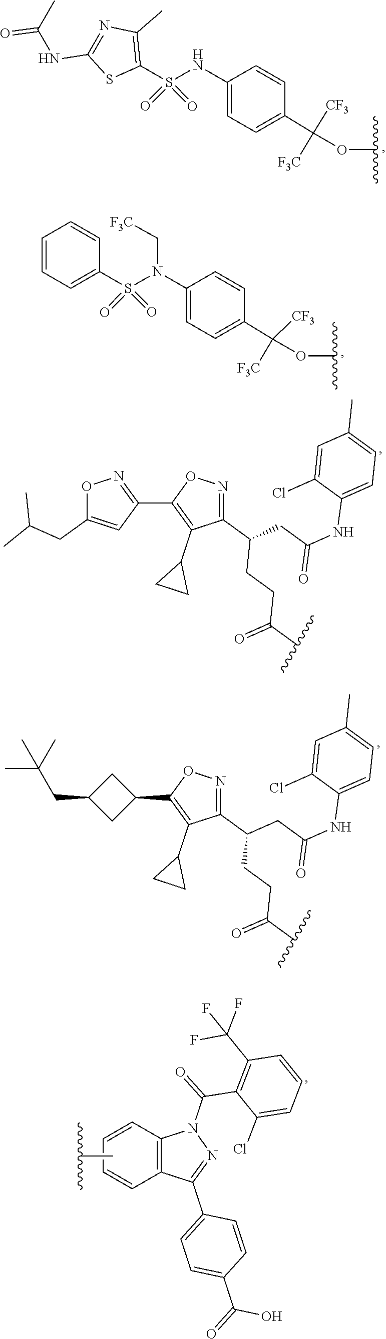

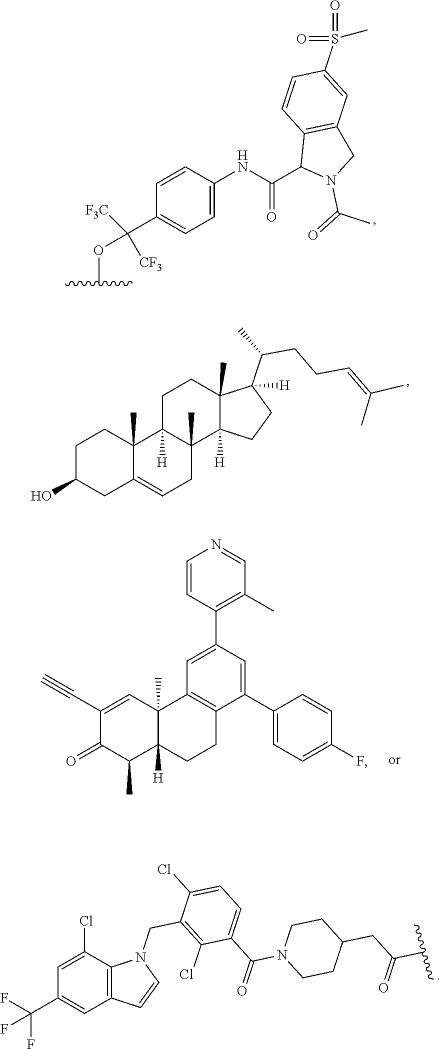

In some embodiments, the DP is indoleamine 2,3-dioxygenase 1 (IDO1) and the B-DP is a IDO1 inhibitor (e.g., see Ute F. Röhrig, et al., Journal of Medicinal Chemistry 2021 64 (24), 17690-17705; Prendergast G C, et al., Discovery of IDO1 Inhibitors: From Bench to Bedside. Cancer Res. 2017 Dec. 15; 77(24):6795-6811). In some embodiments, the B-DP is a IDO1 inhibitor that has one of the following structures:

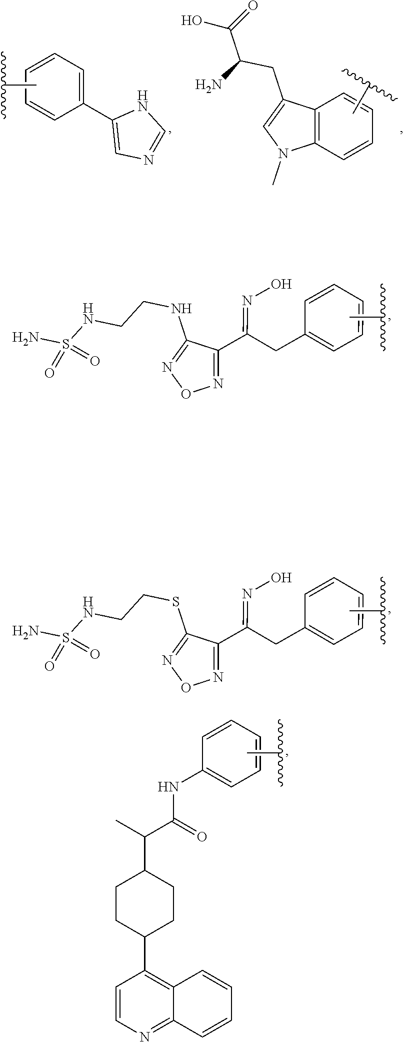

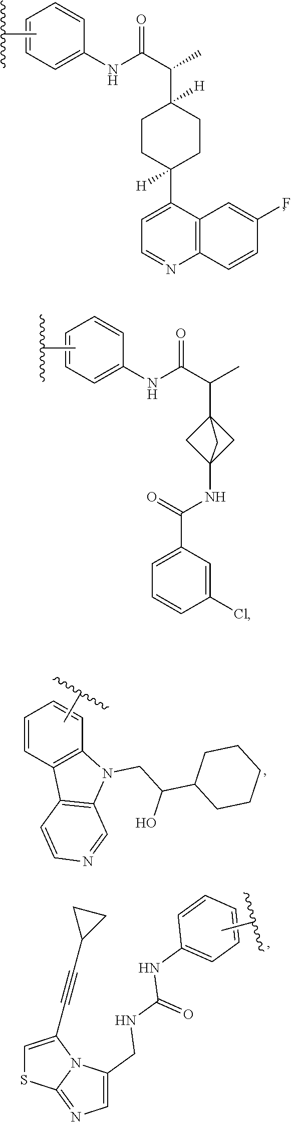

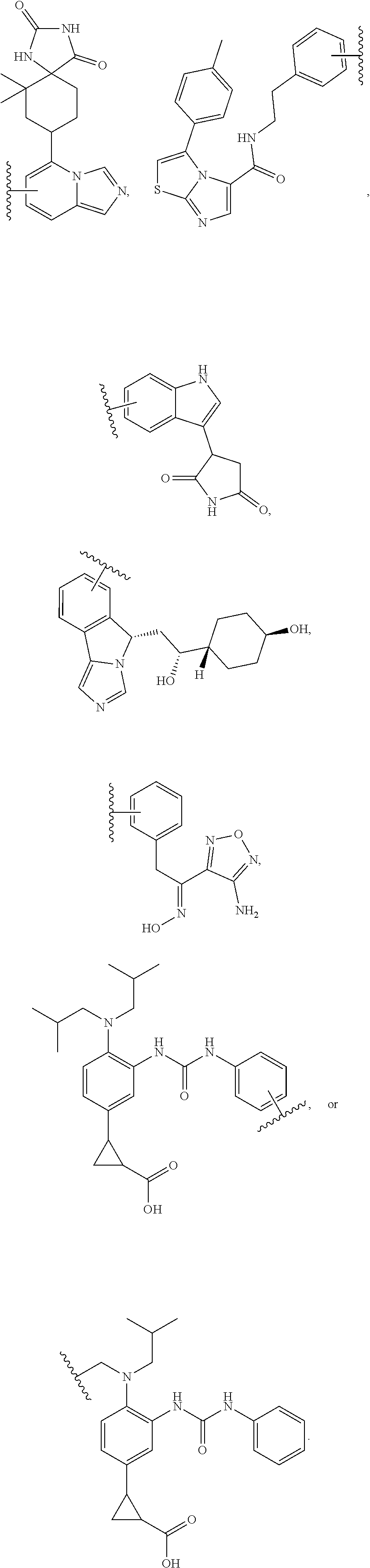

In some embodiments, the linker is absent.

In some embodiments, the linker has a prescribed length thereby linking the SBDDP and the BDP while allowing an appropriate distance therebetween.

In some embodiments, the linker is flexible. In some embodiments, the linker is rigid.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the use of trivalent linkers allow for the preparation of heterotrifunctional compounds comprising one silent binder to a DDP and two binders to a disease protein, or two silent binders to a DDP and one binder to a disease protein, thereby simultaneously binding a disease protein and a DDP and forming a productive ternary complex. In some embodiments, such heterotrifunctional compounds would result in more sustained and more potent anticancer activity.

In some embodiments, the linker comprises one or more linear structures, one or more non-linear structures, one or more branched structures, one or more cyclic structures, one or more flexible moieties, one or more rigid moieties, or combinations thereof.

In some embodiments, the linker comprises one or more amino acid residues. In some embodiments, the linker comprises 1 to 3, 1 to 5, 1 to 10, 5 to 10, or 5 to 20 amino acid residues. In some embodiments, one or more amino acids of the linker are unnatural amino acids. In some embodiments, the linker comprises a peptide linkage. The peptide linkage comprises L-amino acids and/or D-amino acids.

In some embodiments, the linker has 1 to 100 atoms, 1 to 50 atoms, 1 to 30 atoms, 1 to 20 atoms, 1 to 15 atoms, 1 to 10 atoms, or 1 to 5 atoms in length. In some embodiments, the linker has 1 to 10 atoms in length. In some embodiments, the linker has 1 to 20 atoms in length.

In some embodiments, the linker comprises flexible and/or rigid regions.

In some embodiments, the linker is L, wherein L is absent or

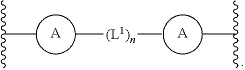

In some embodiments, L is absent. In some embodiments, L is

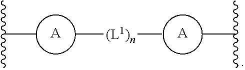

In some embodiments, L comprises substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted 3- to 10-membered heterocycloalkyl, substituted or unsubstituted aryl, substituted or unsubstituted heteroaryl, or combinations thereof.

In some embodiments, L is absent or

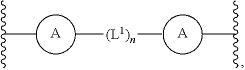

wherein:
each A is independently absent, substituted or unsubstituted monocyclic C₃-C₁₀cycloalkyl, substituted or unsubstituted bridged bicyclic C₃-C₁₀cycloalkyl, substituted or unsubstituted fused bicyclic C₃-C₁₀cycloalkyl, substituted or unsubstituted spiro bicyclic C₃-C₁₀cycloalkyl, substituted or unsubstituted monocyclic 3- to 10-membered heterocycloalkyl, substituted or unsubstituted bridged bicyclic 3- to 10-membered heterocycloalkyl, substituted or unsubstituted fused bicyclic 3- to 10-membered heterocycloalkyl, substituted or unsubstituted spiro bicyclic 3- to 10-membered heterocycloalkyl, substituted or unsubstituted aryl, or substituted or unsubstituted heteroaryl, wherein each A is independently unsubstituted or substituted with x R^{2b};
each L is independently absent,

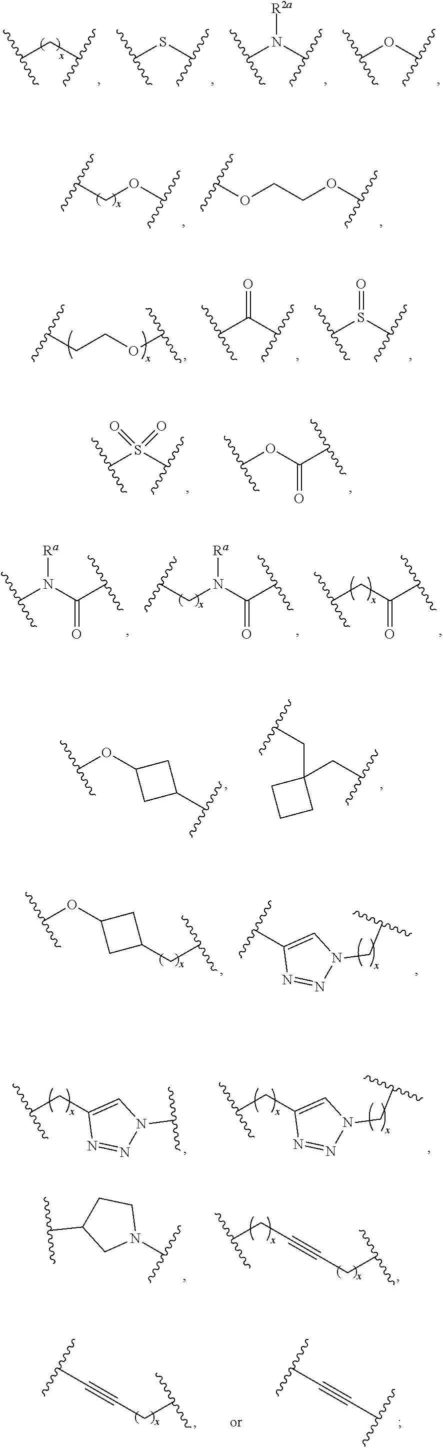

wherein each L¹ is independently unsubstituted or substituted with x R^{2b};
n is 1, 2, 3, 4, 5, or 6;
each x is independently 1, 2, 3, 4, 5, 6, 7, or 8;
each R^{2a} is independently hydrogen or substituted or unsubstituted C₁-C₆alkyl; and
each R^{2b} is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, —OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b});
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, each A is independently absent,

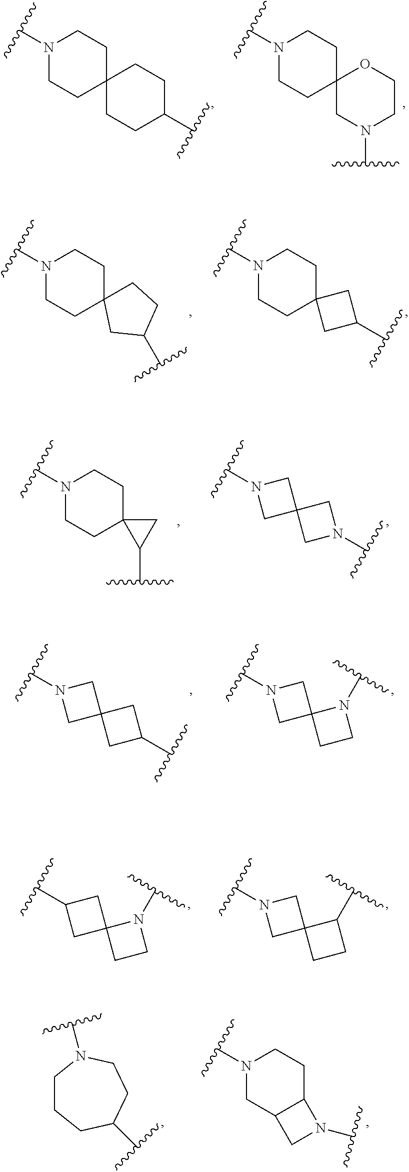

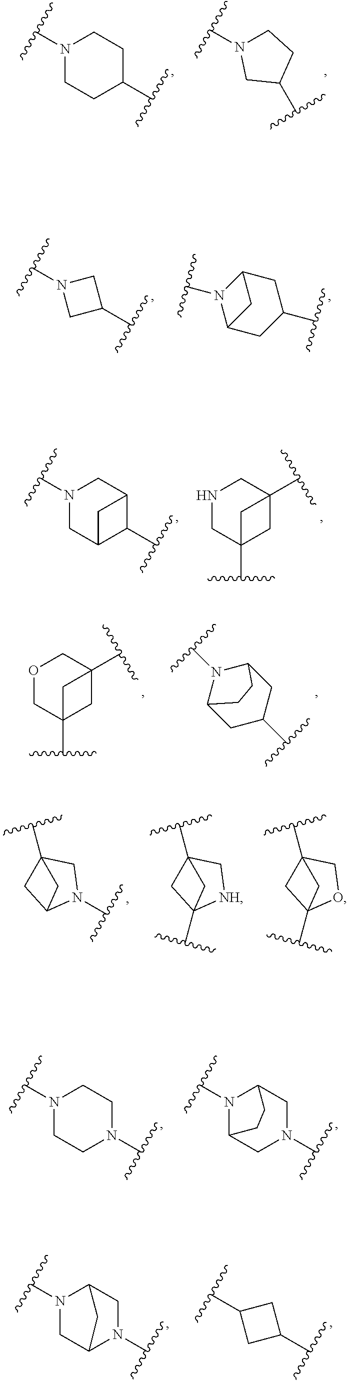

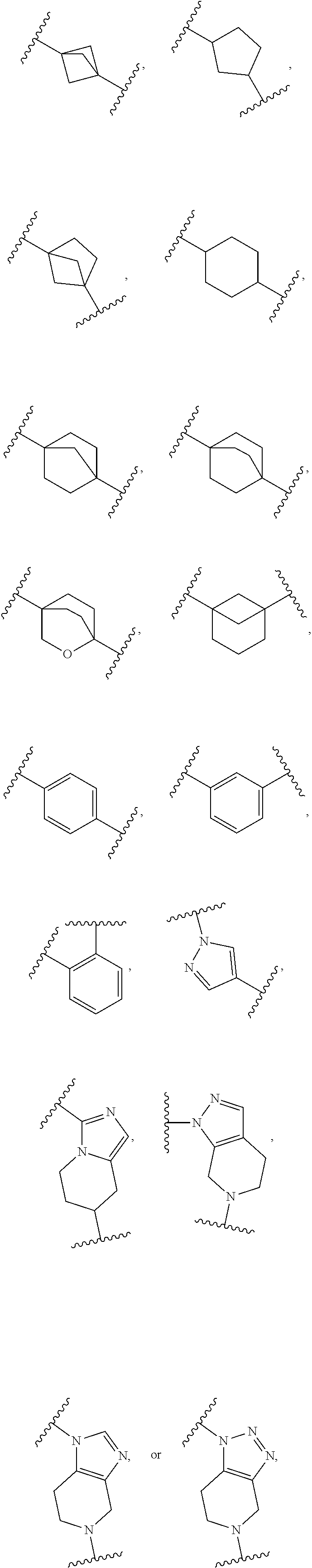

or wherein each A is independently unsubstituted or substituted with x R^{2b}; (L¹)ₙ is absent,

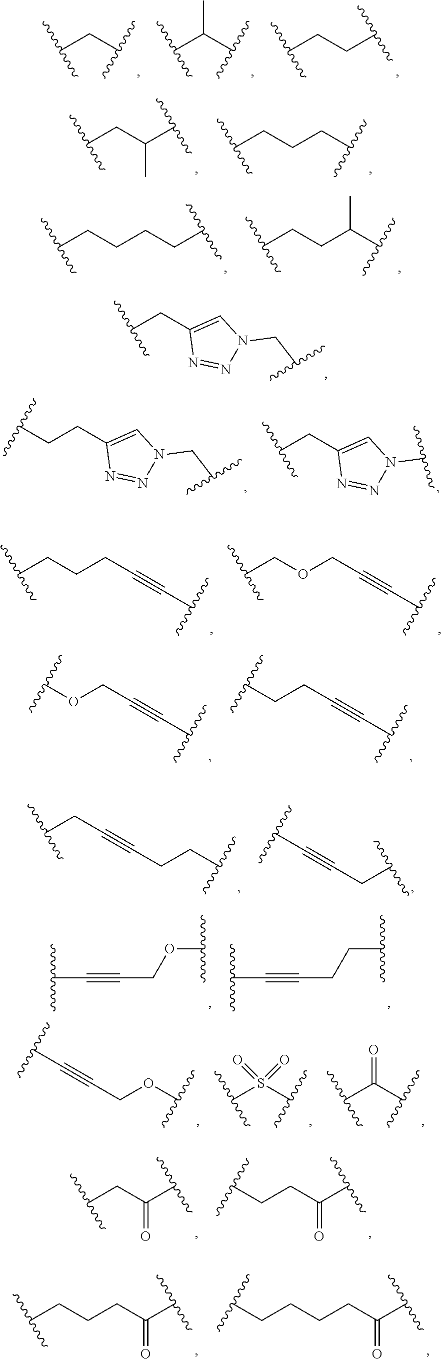

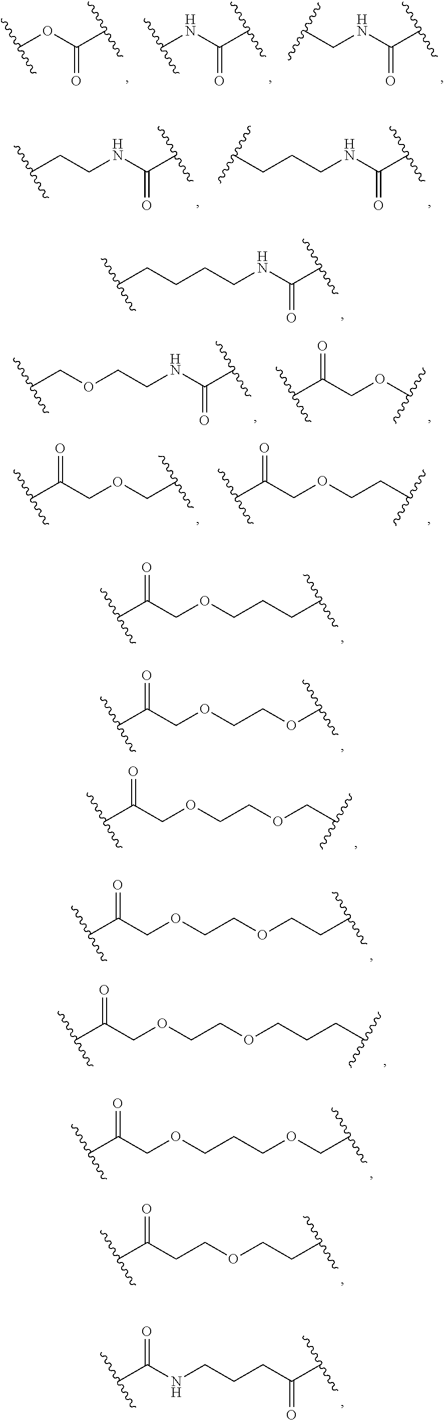

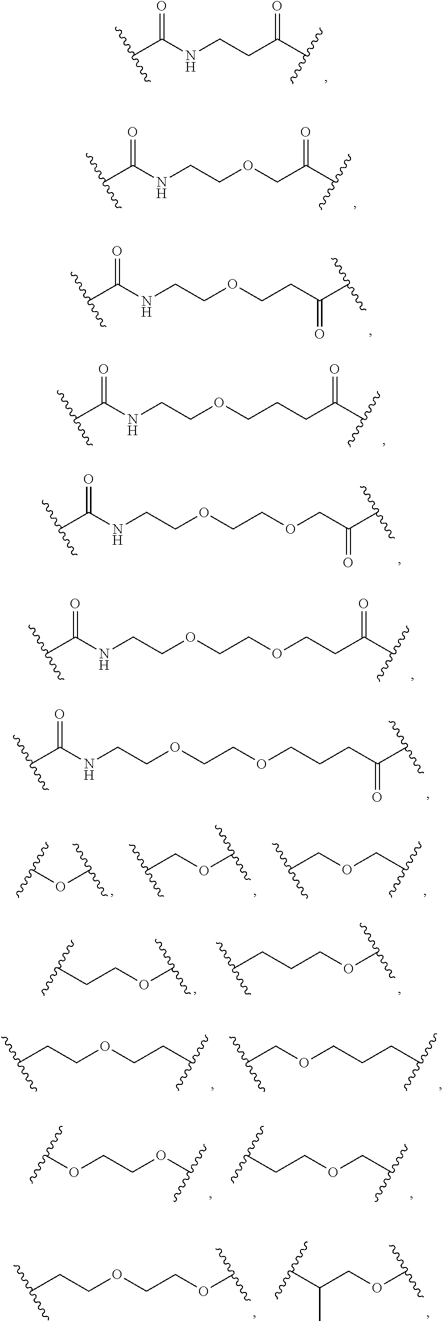

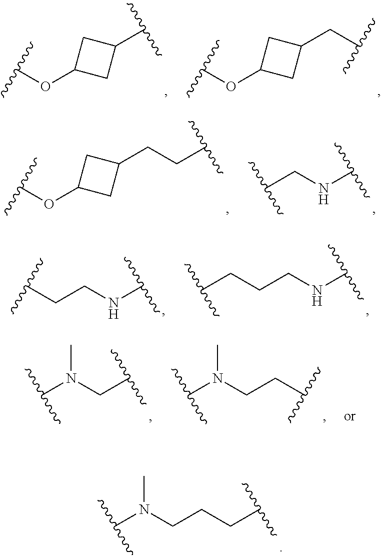

In some embodiments, L is absent or

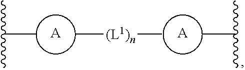

wherein:
each A is independently absent,

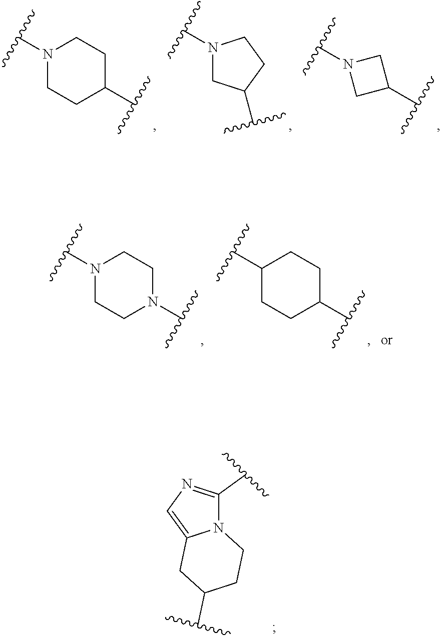 [SMILES: *C1CCN(*)CC1 ; *C1CCN(*)C1 ; *C1CN(*)C1 ; *N1CCN(*)CC1 ; *C1CCC(*)CC1 ; *c1ncc2n1CCC(*)C2]

each L¹ is independently absent,

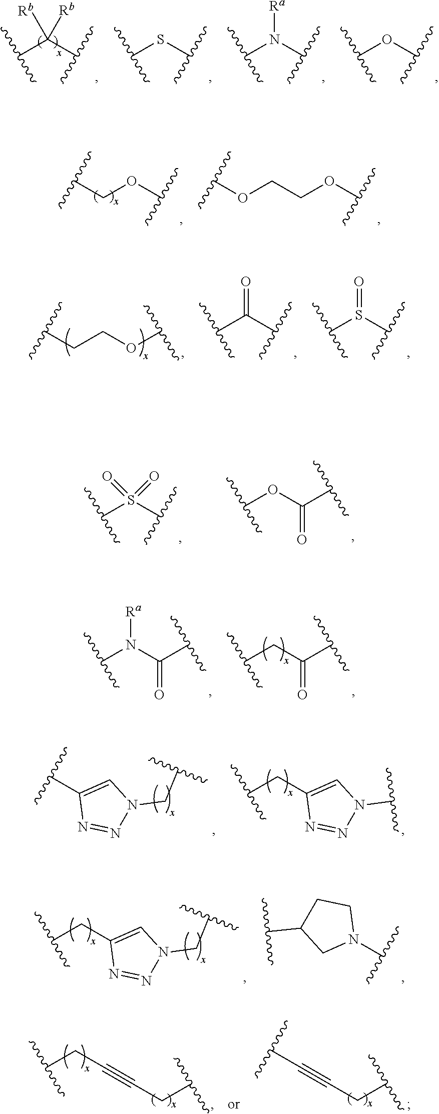

n is 1, 2, or 3;
each x is independently 1, 2, 3, 4, 5, or 6;
each R^{a} is independently hydrogen or substituted or unsubstituted C₁-C₆alkyl; and
each R^{b} is independently hydrogen, halogen, or substituted or unsubstituted C₁-C₆alkyl.

In some embodiments, each A is independently absent, absent,

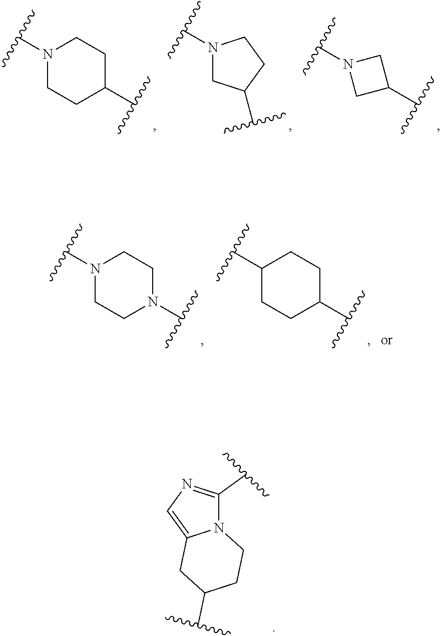 [SMILES: *C1CCN(*)CC1 ; *C1CCN(*)C1 ; *C1CN(*)C1 ; *N1CCN(*)CC1 ; *C1CCC(*)CC1 ; *c1ncc2n1CCC(*)C2]

In some embodiments, each A is independently

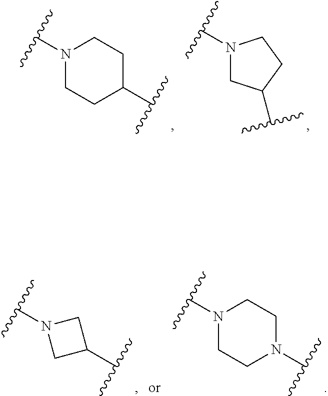 [SMILES: *C1CCN(*)CC1 ; *C1CCN(*)C1 ; *C1CN(*)C1 ; *N1CCN(*)CC1]

In some embodiments, each A is independently absent,

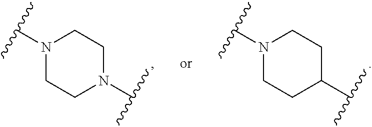 [SMILES: *N1CCN(*)CC1 ; *C1CCN(*)CC1]

In some embodiments, each absent,

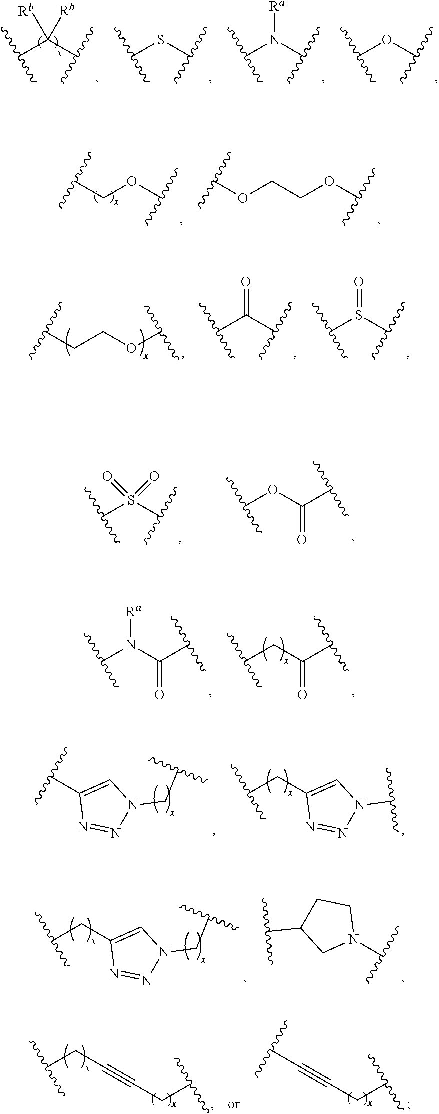

n is 1, 2, or 3;
each x is independently 1, 2, 3, 4, 5, or 6;
each R^{a} is independently hydrogen or substituted or unsubstituted C₁-C₆alkyl; and
each R^{b} is independently hydrogen, halogen, or substituted or unsubstituted C₁-C₆alkyl.

In some embodiments, n is 1. In some embodiments, n is 2. In some embodiments, n is 3.

In some embodiments, x is 1. In some embodiments, x is 2. In some embodiments, x is 3. In some embodiments, x is 4. In some embodiments, x is 5. In some embodiments, x is 6.

In some embodiments, R^{a} is hydrogen. In some embodiments, each R^{a} is independently substituted or unsubstituted C₁-C₆alkyl. In some embodiments, each R^{a} is independently hydrogen, methyl, ethyl, propyl, iso-propyl, n-butyl, iso-butyl, or tert-butyl. In some embodiments, each R^{a} is independently hydrogen or methyl. In some embodiments, R^{a} is methyl.

In some embodiments, R^{b} is hydrogen. In some embodiments, each R^{b} is independently substituted or unsubstituted C₁-C₆alkyl. In some embodiments, each R^{b} is independently hydrogen, methyl, ethyl, propyl, iso-propyl, n-butyl, iso-butyl, or tert-butyl. In some embodiments, each R^{b} is independently hydrogen or methyl. In some embodiments, R^{b} is methyl.

In some embodiments, (L¹)ₙ is absent,

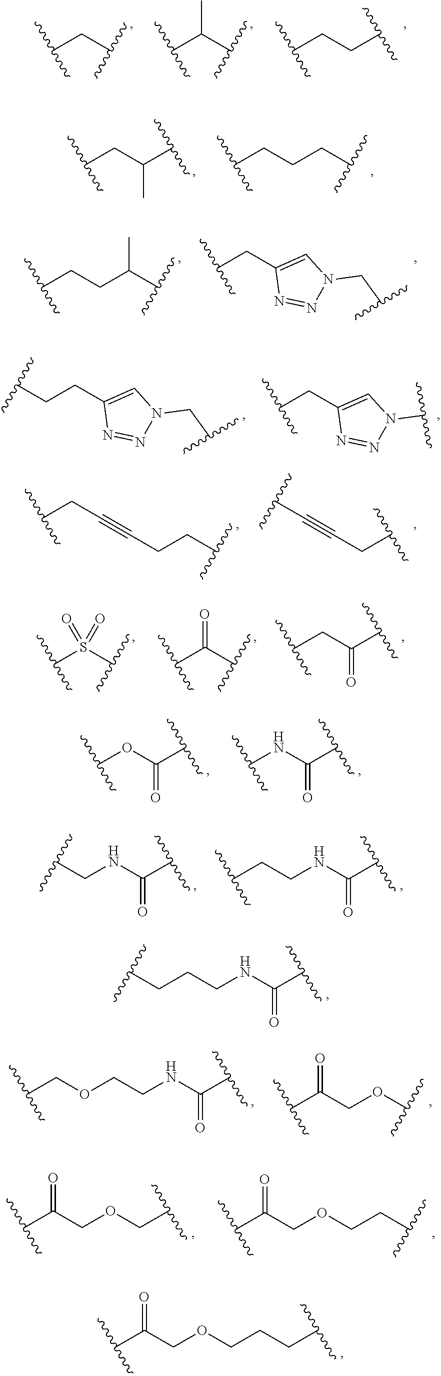

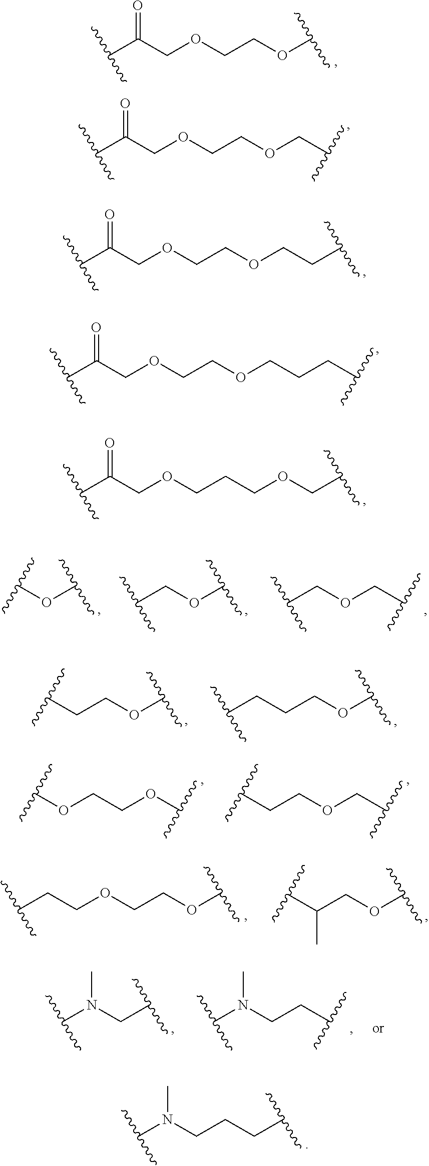

In some embodiments, (L¹)ₙ is absent,

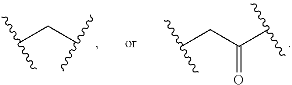

In some embodiments, L is

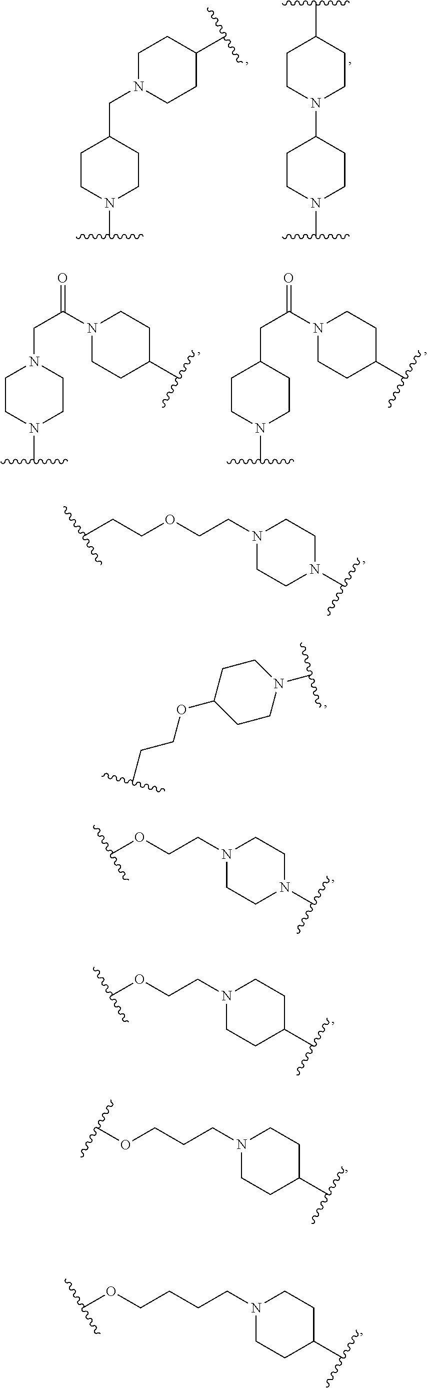

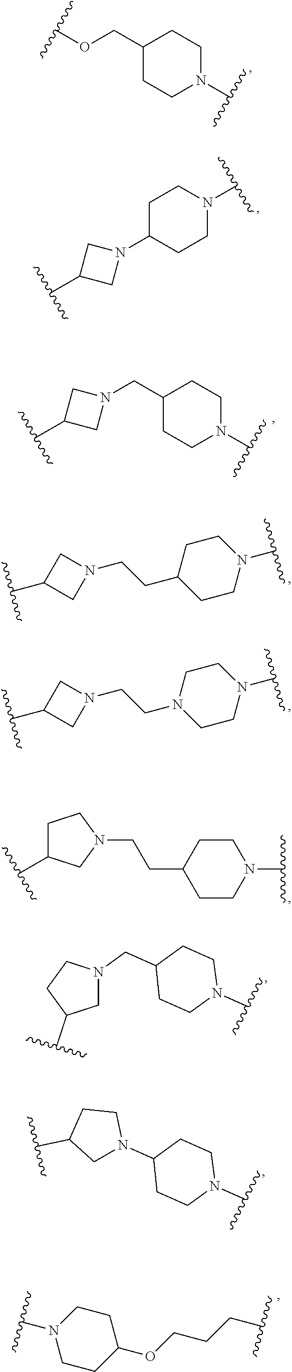

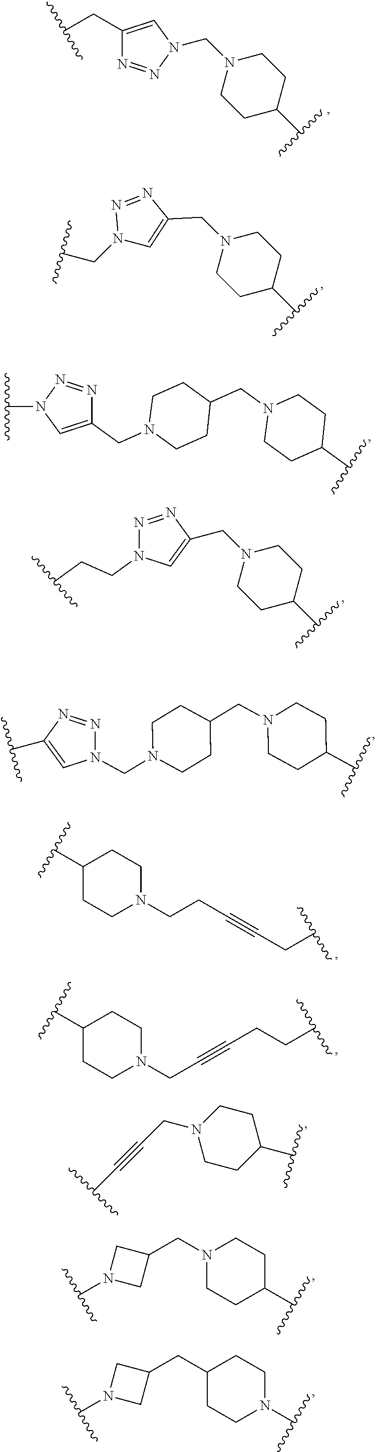

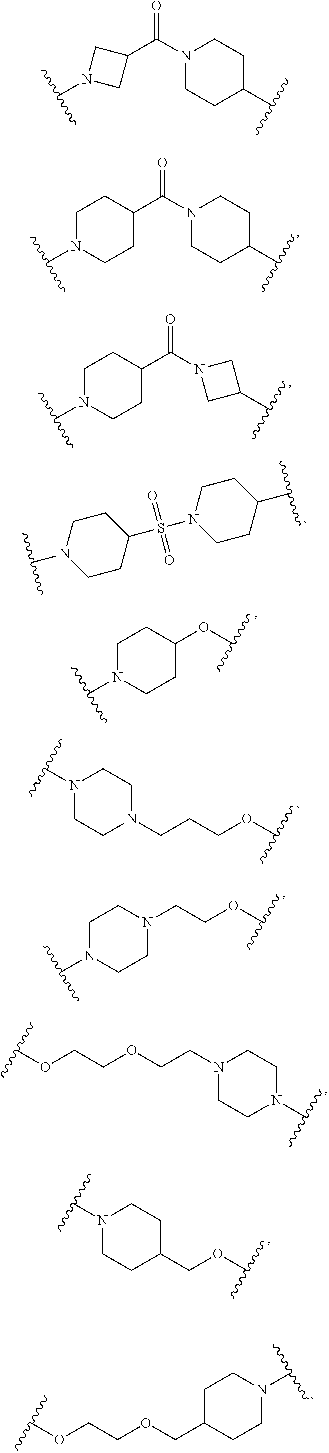

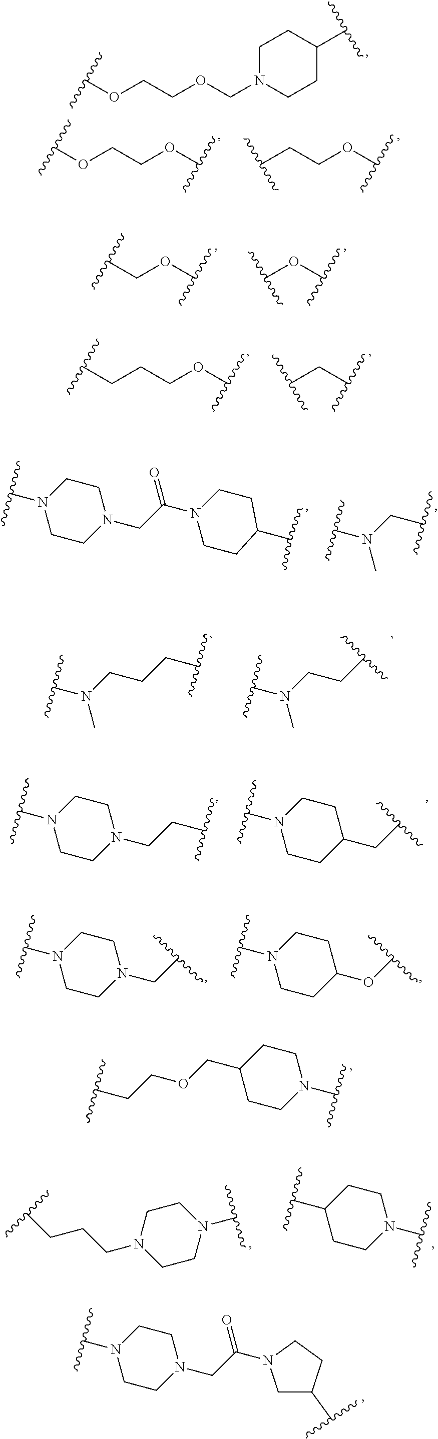

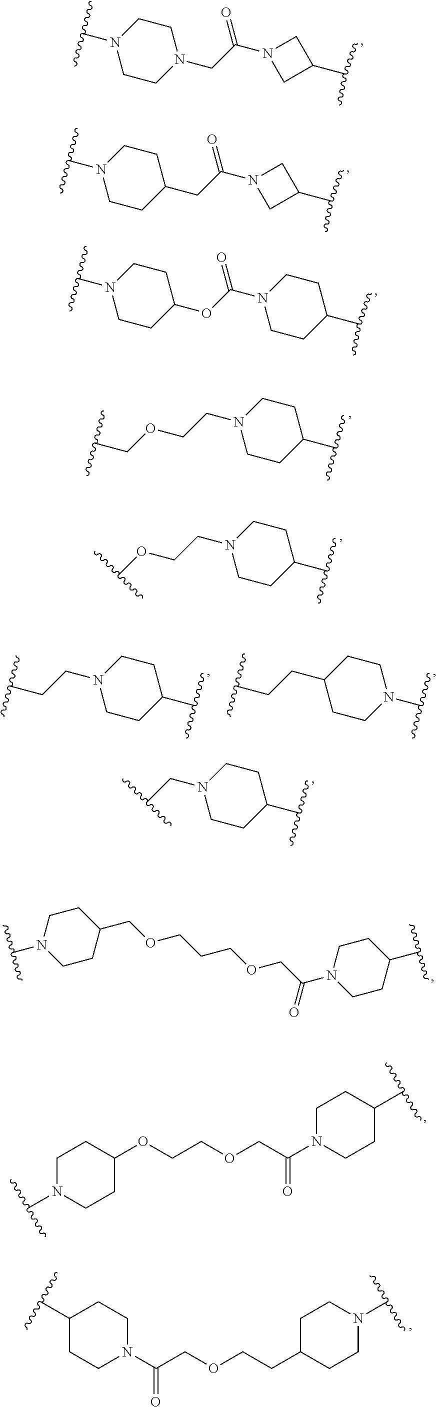

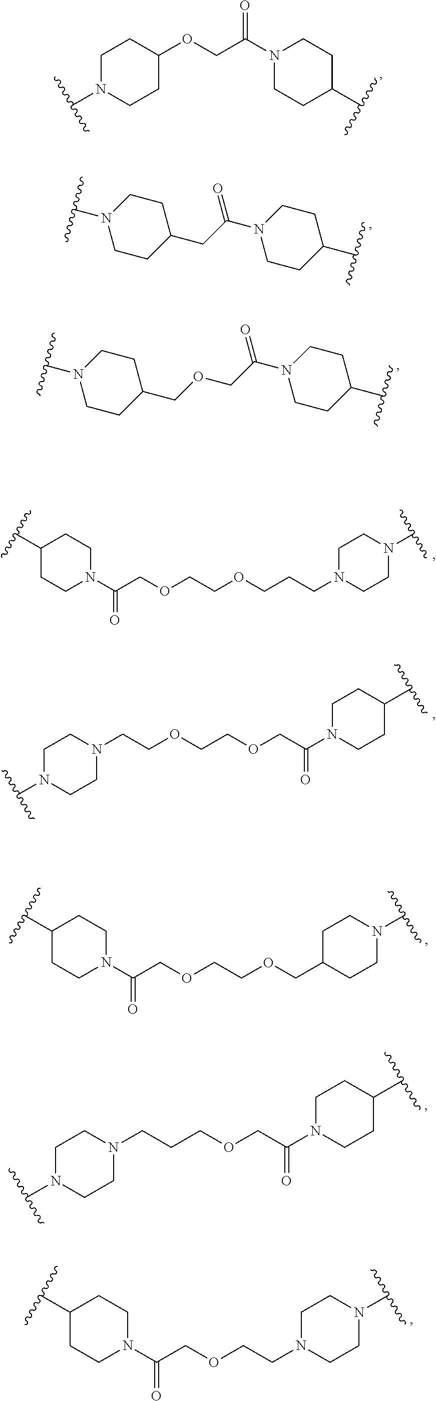

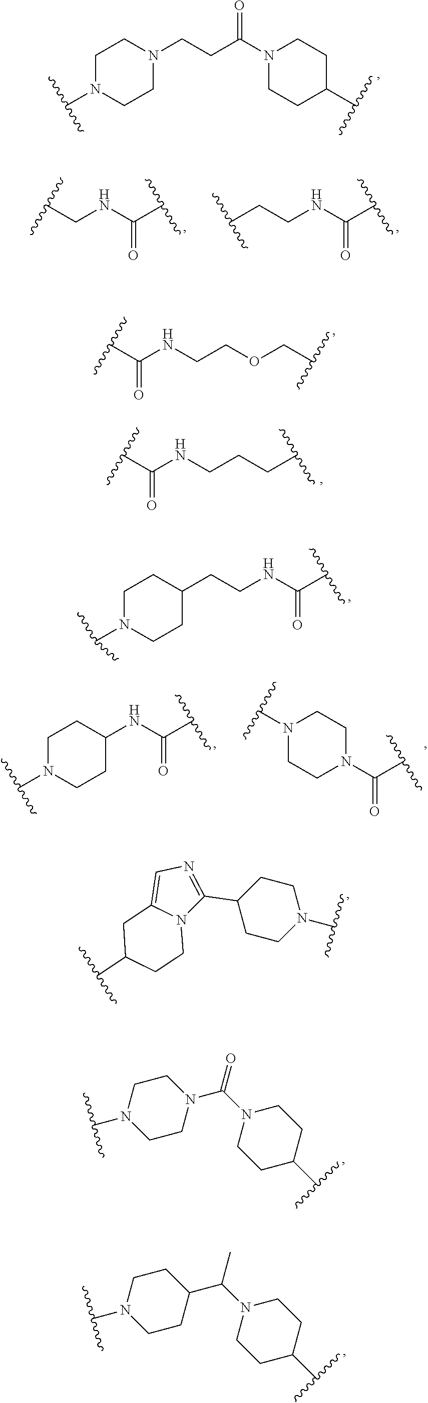

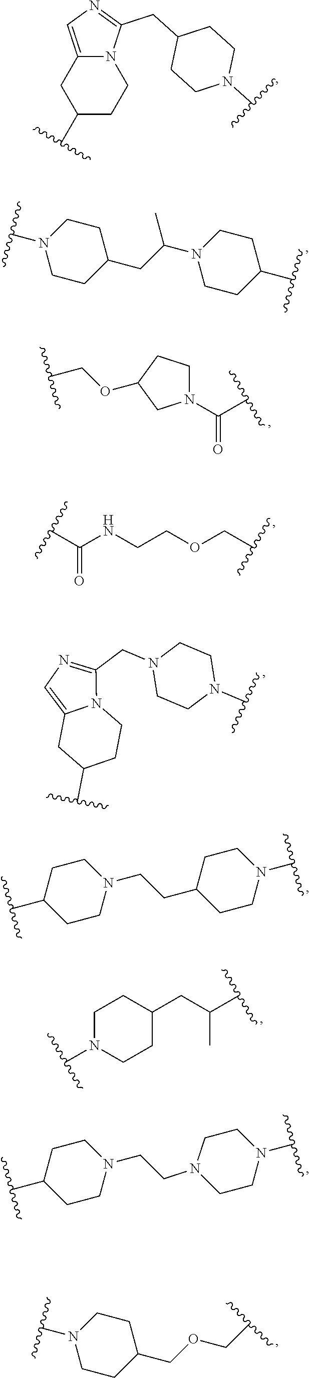

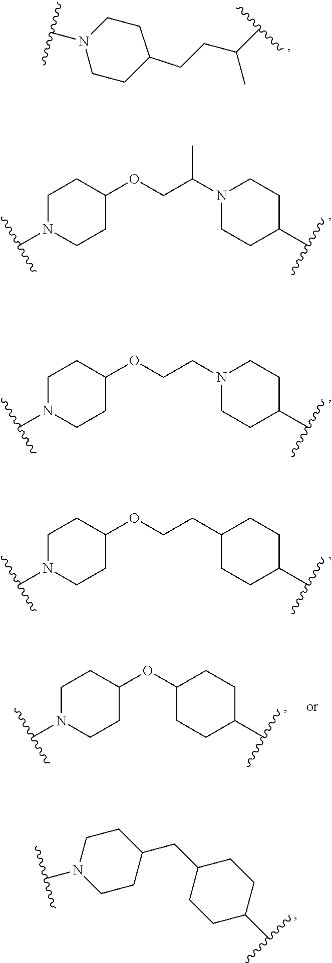 [SMILES: *C(C)CCC1CCN(*)CC1 ; *C1CCN(C(C)COC2CCN(*)CC2)CC1 ; *C1CCN(CCOC2CCN(*)CC2)CC1 ; *C1CCC(CCOC2CCN(*)CC2)CC1 ; *C1CCC(OC2CCN(*)CC2)CC1 ; *C1CCC(CC2CCN(*)CC2)CC1]

or a pharmaceutically acceptable salt or solvate thereof.

In some embodiments, L is

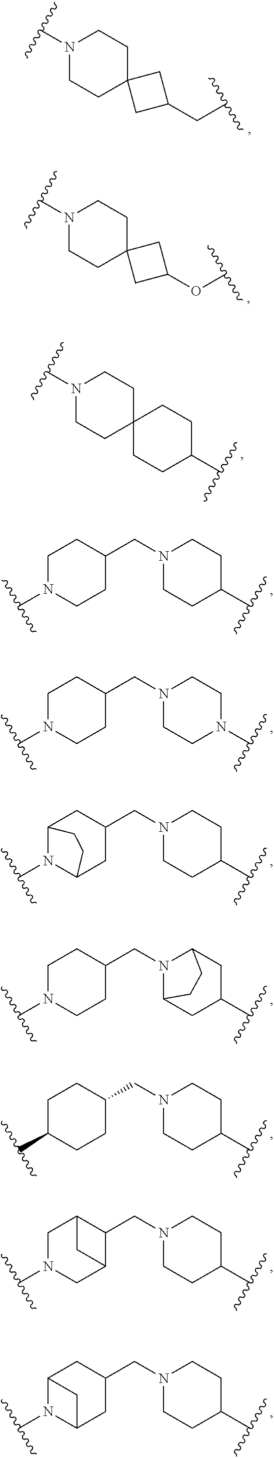

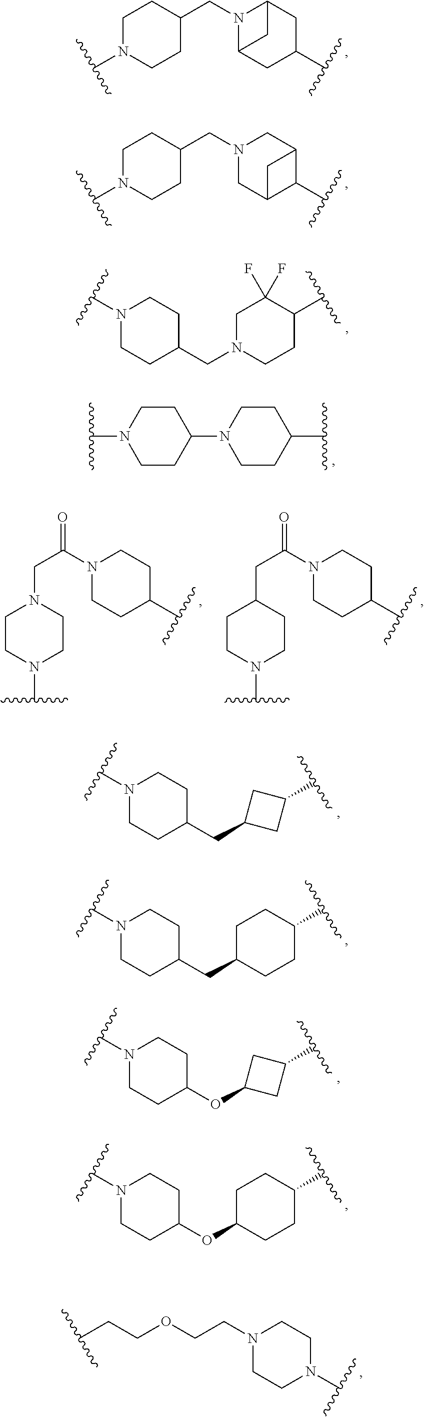

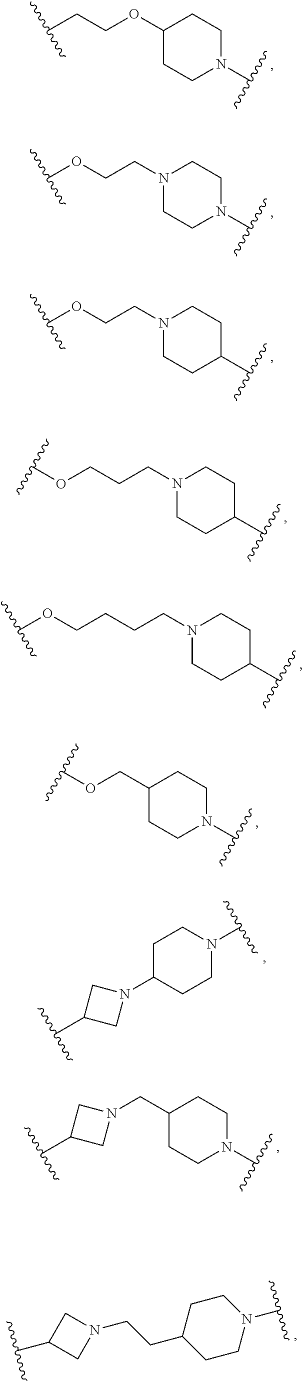

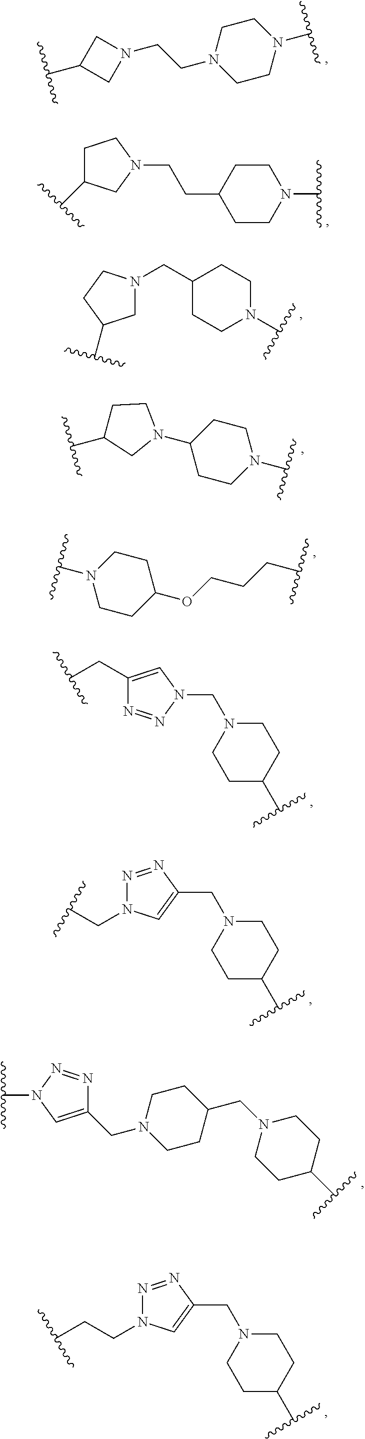

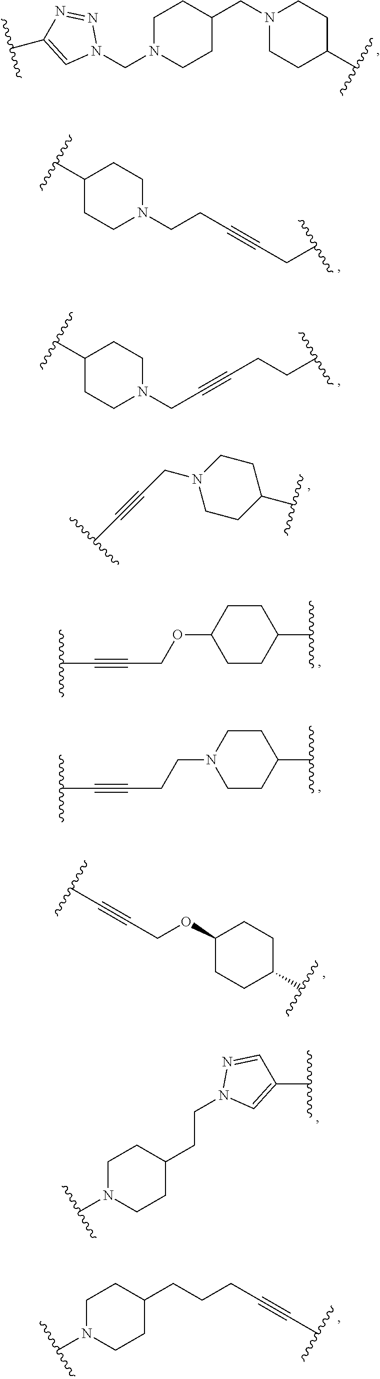

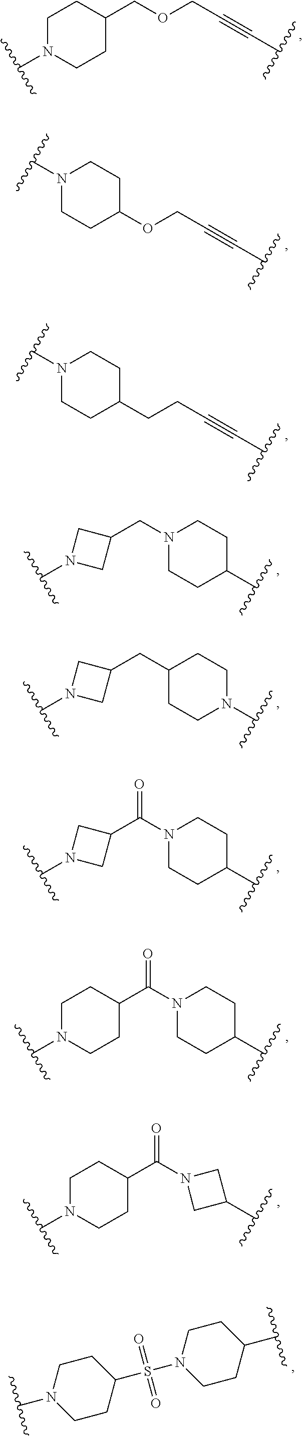

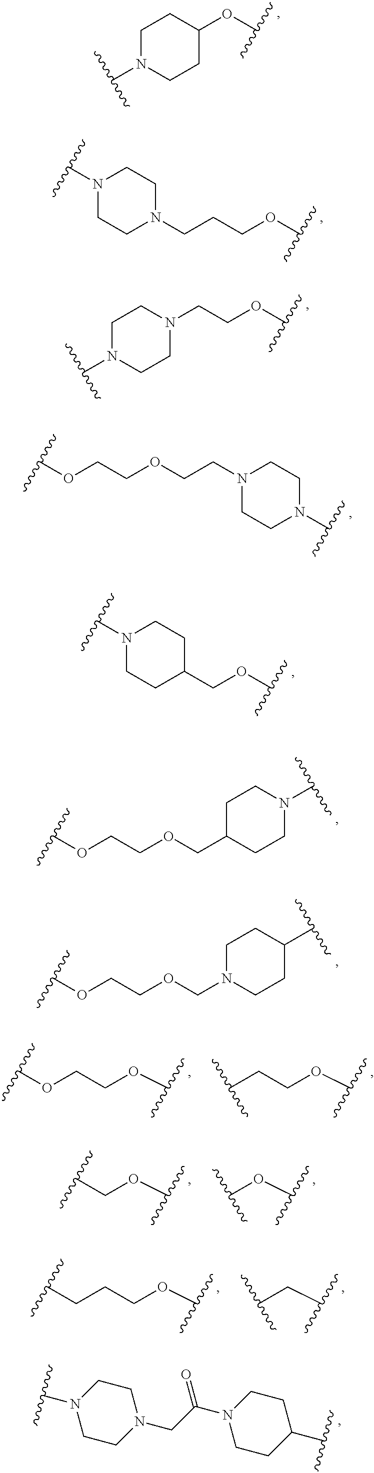

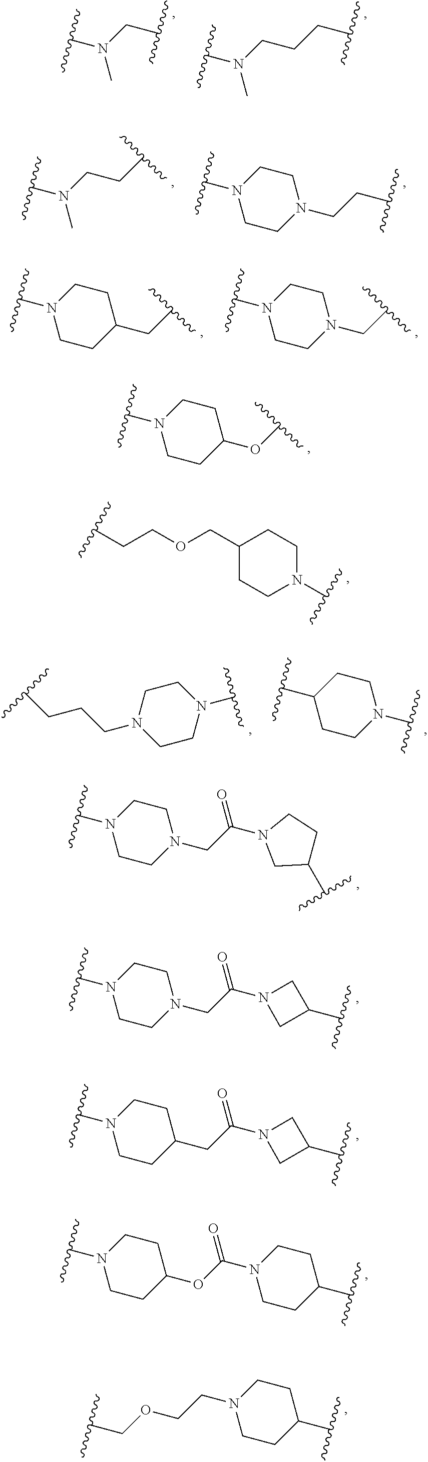

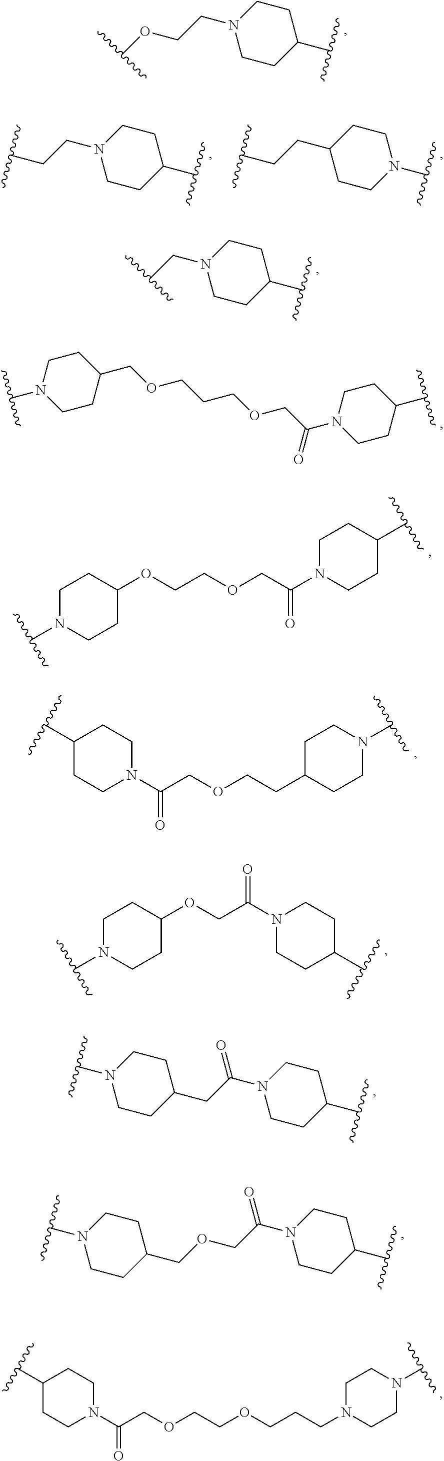

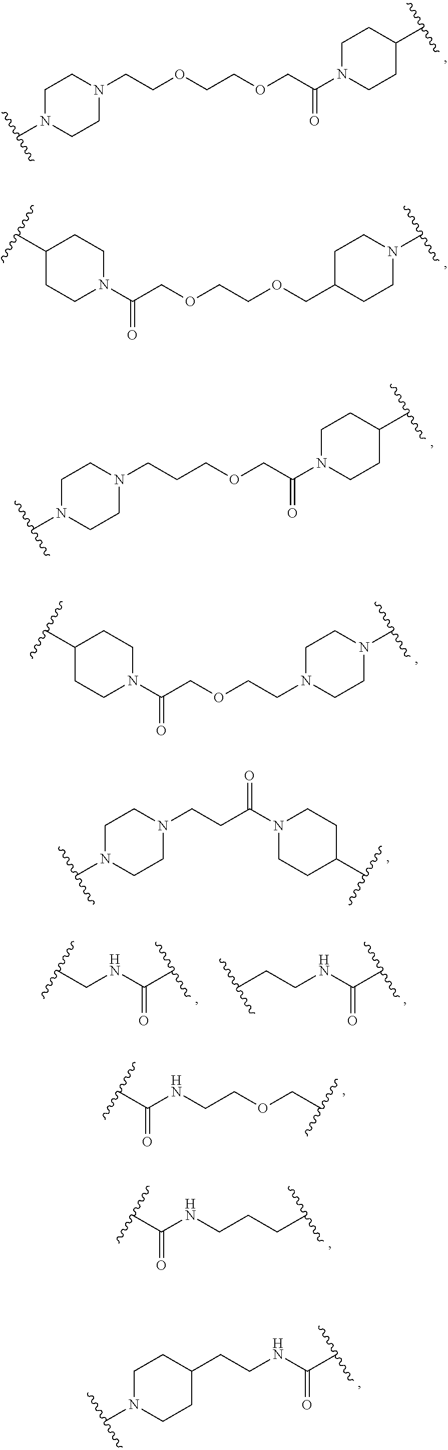

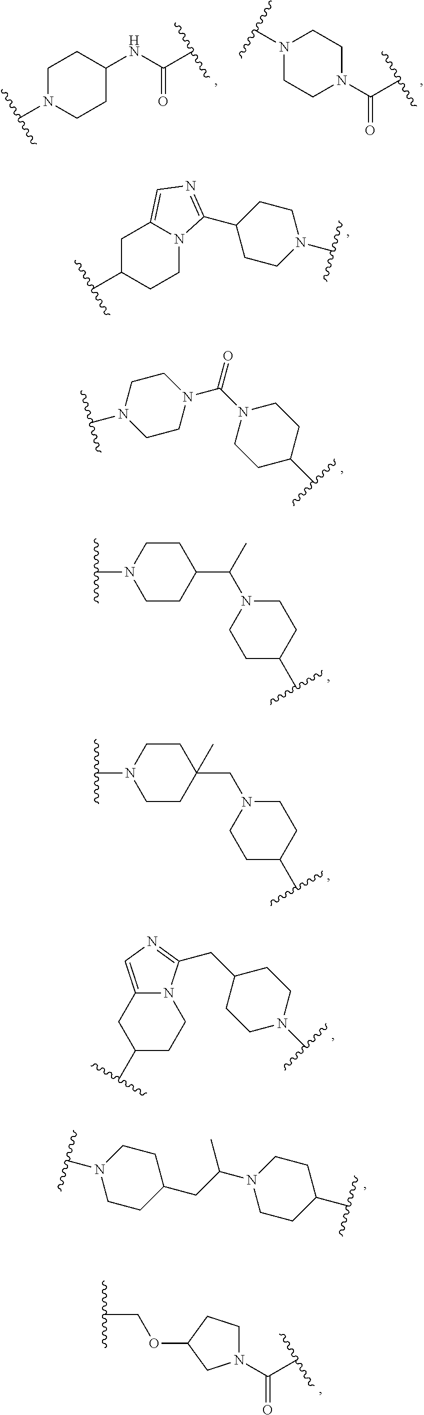

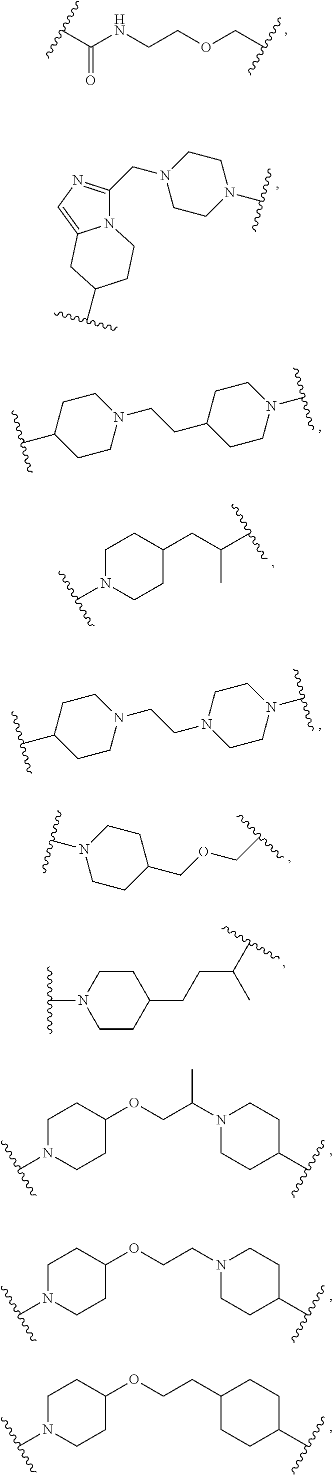

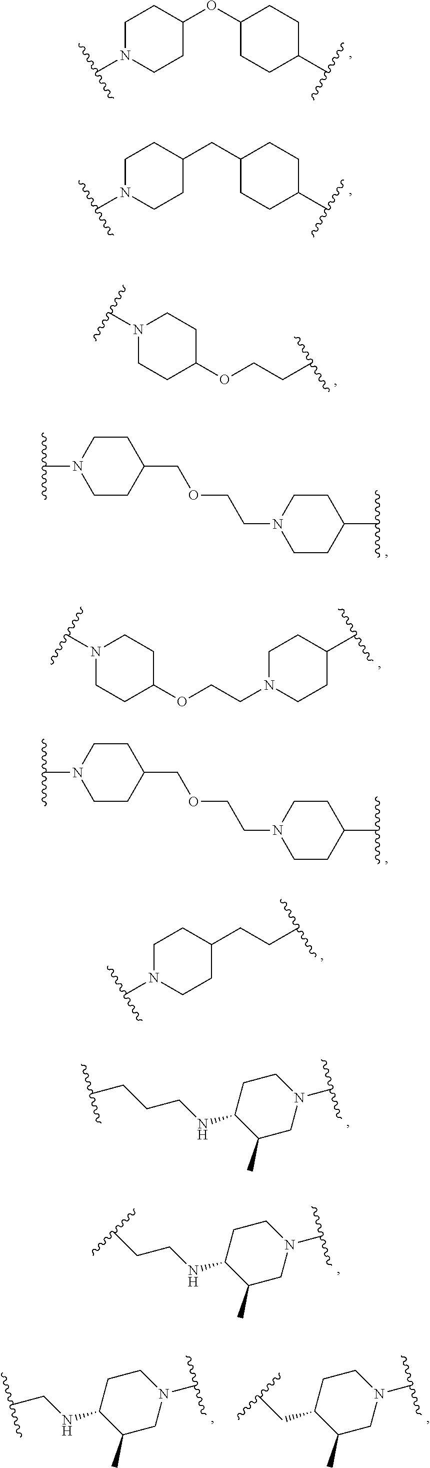

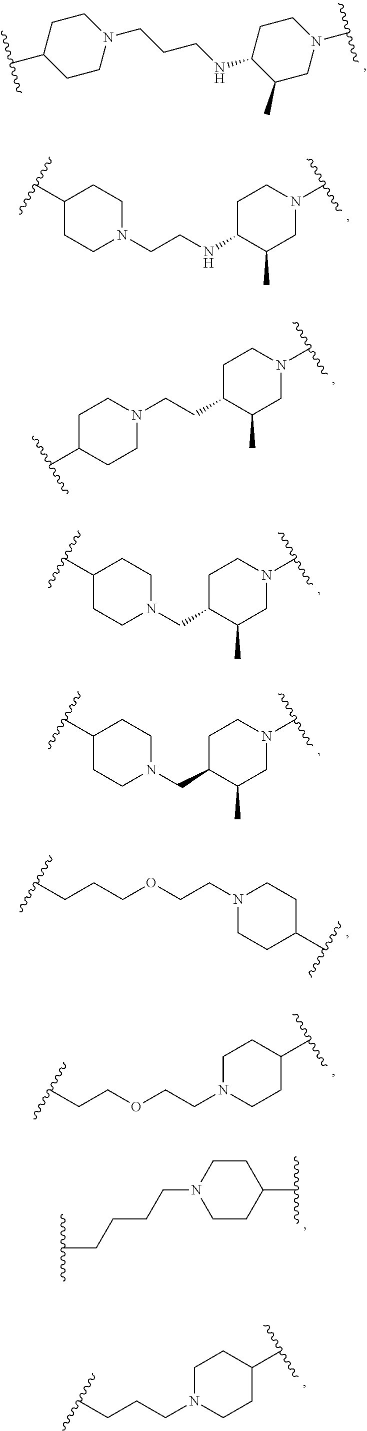

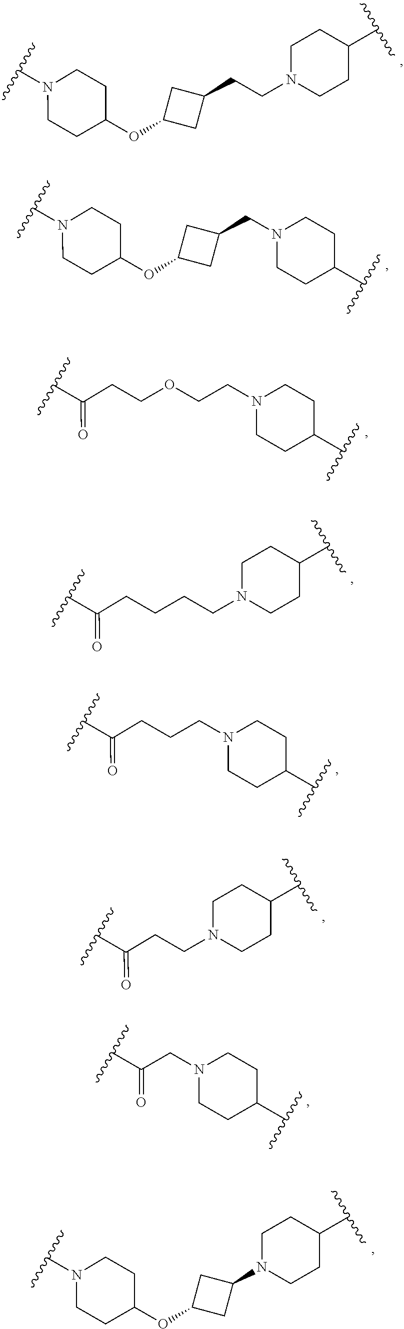

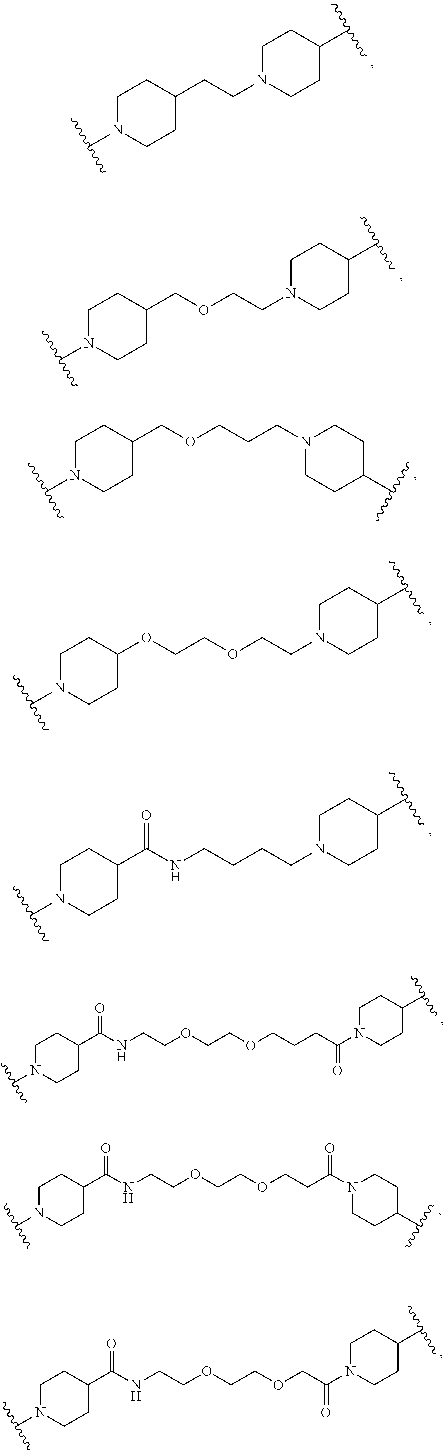

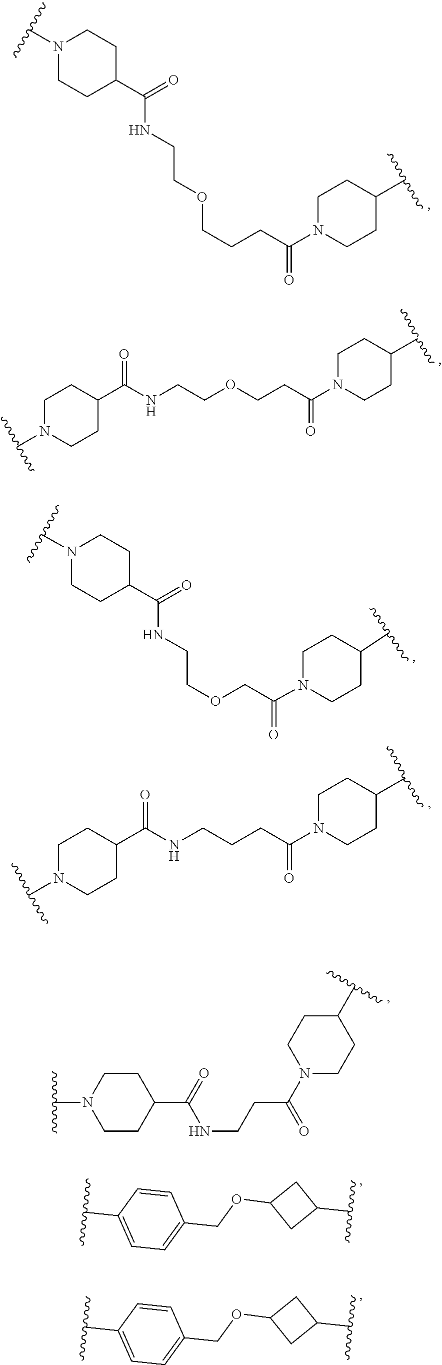

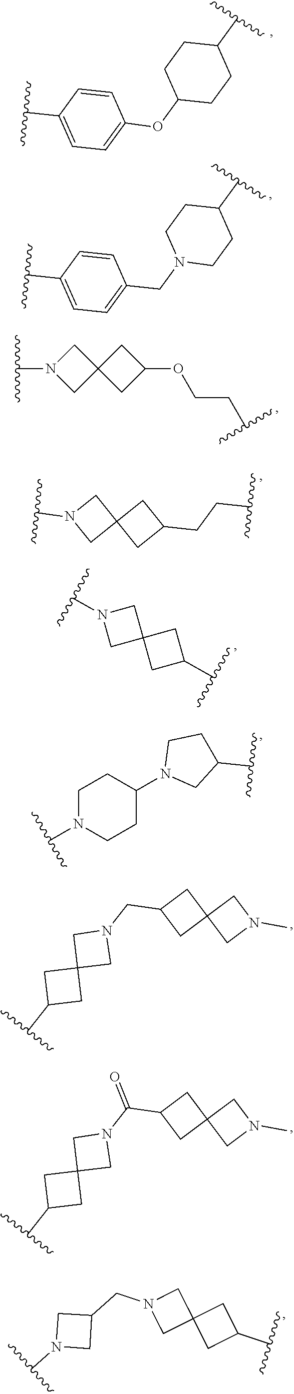

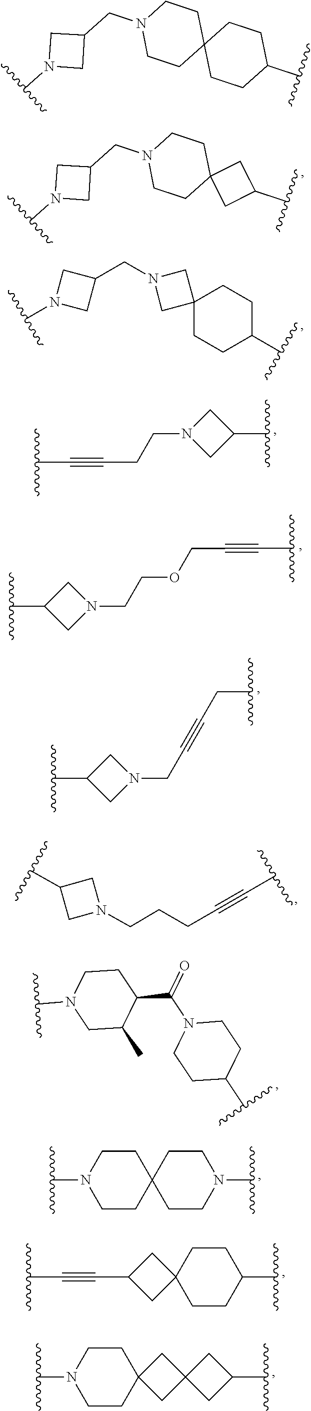

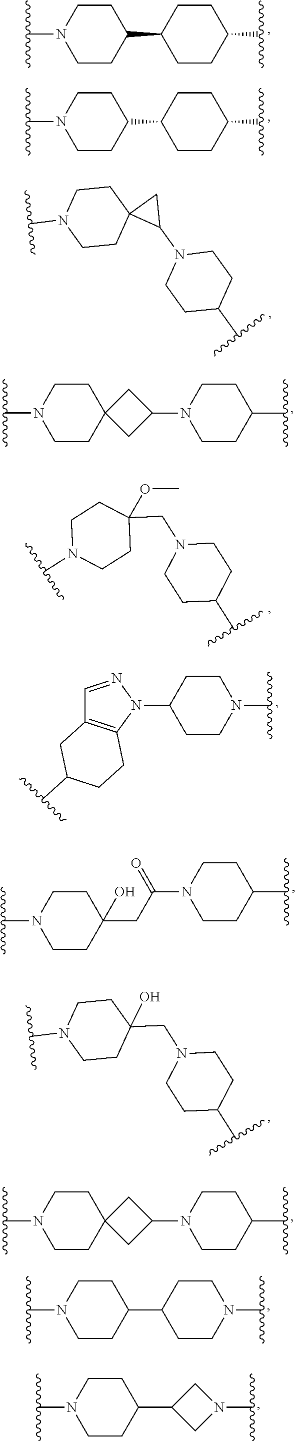

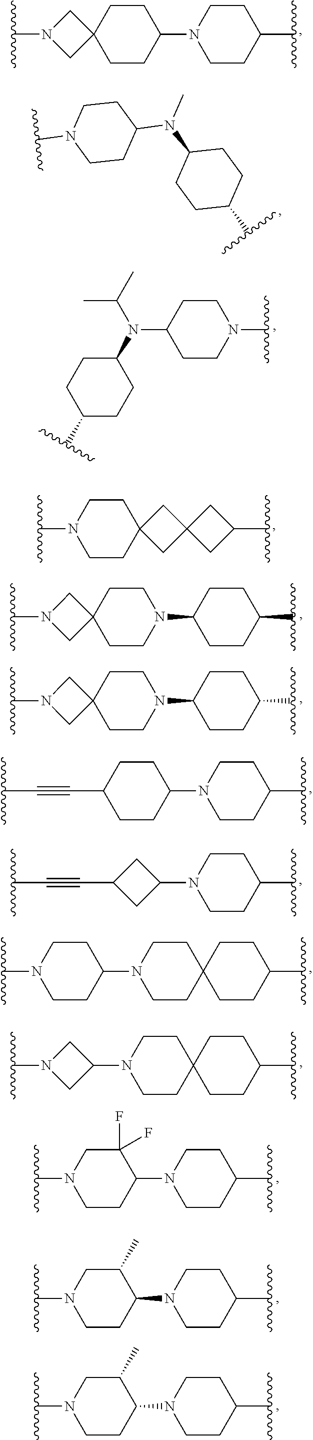

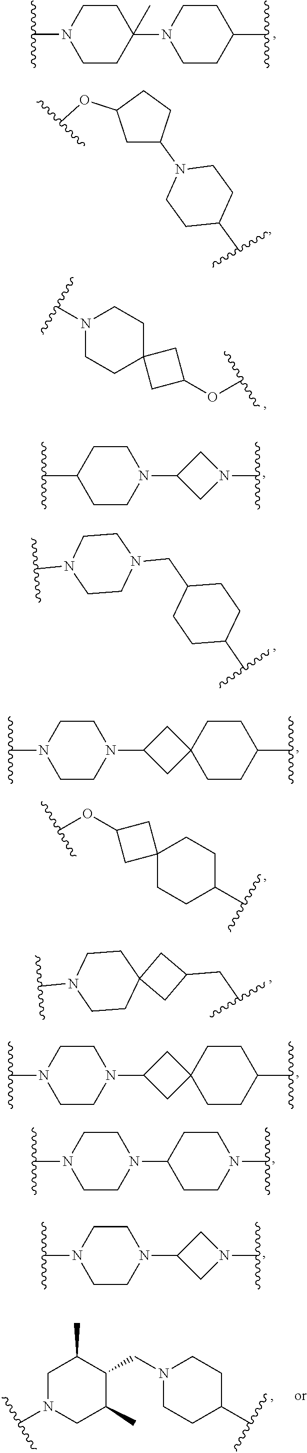

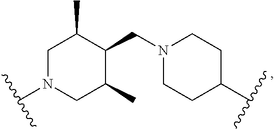 [SMILES: *C1CCN(C[C@@H]2[C@H](C)CN(*)C[C@@H]2C)CC1]

or a pharmaceutically acceptable salt or solvate thereof.

In some embodiments, L has one of the following structures:

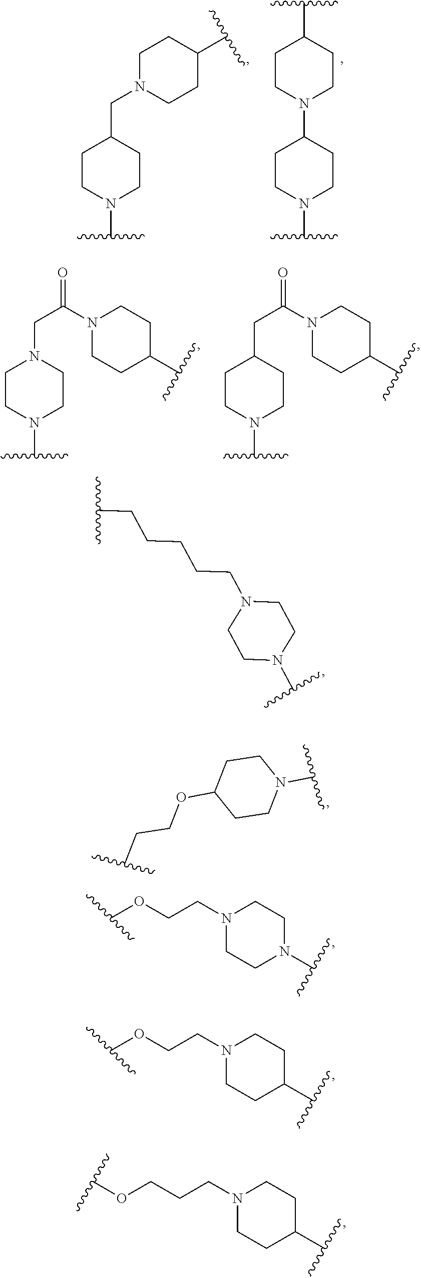

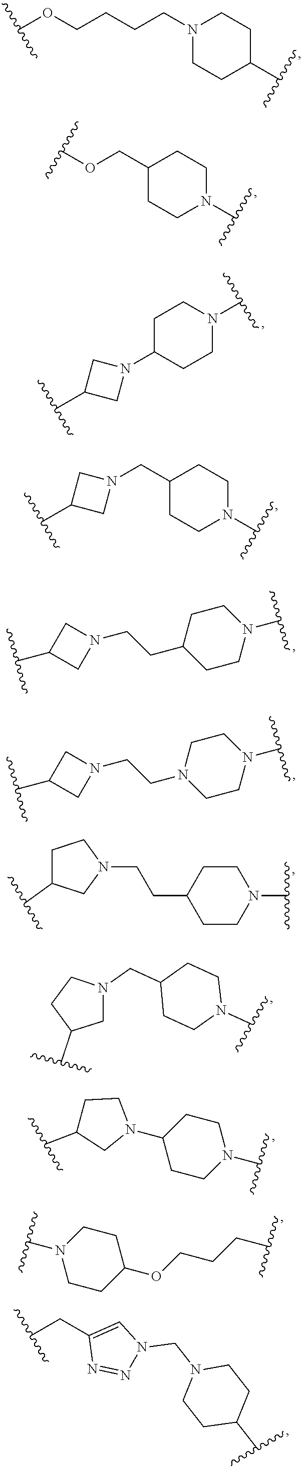

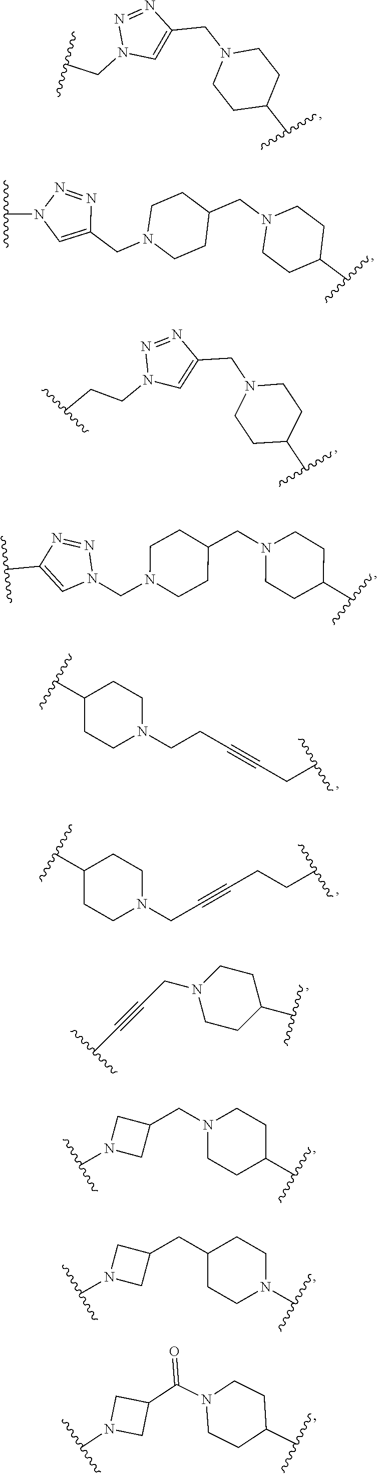

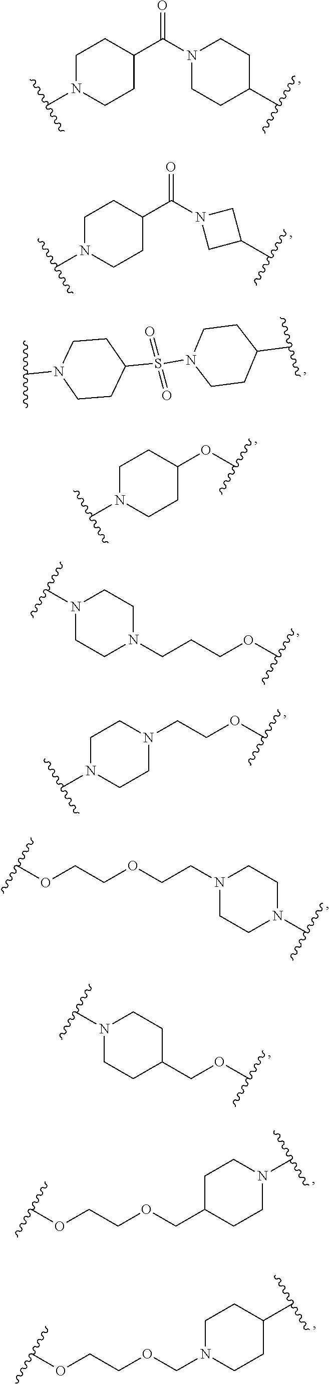

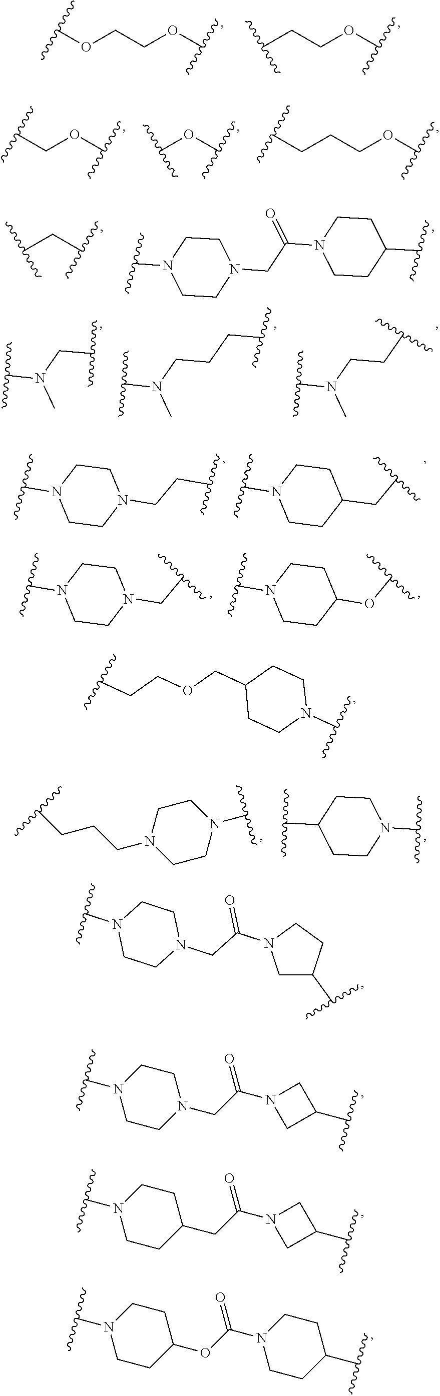

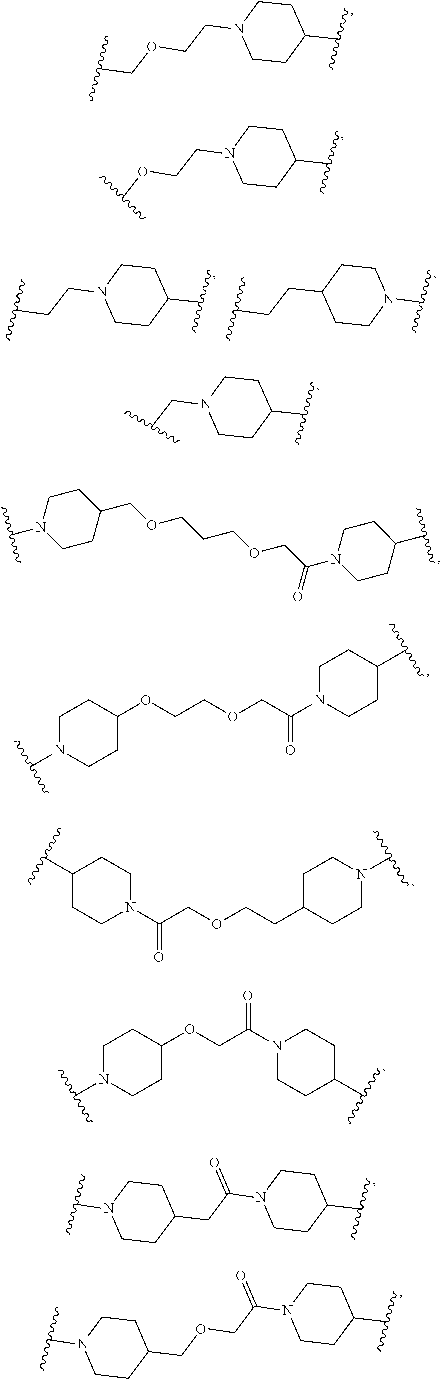

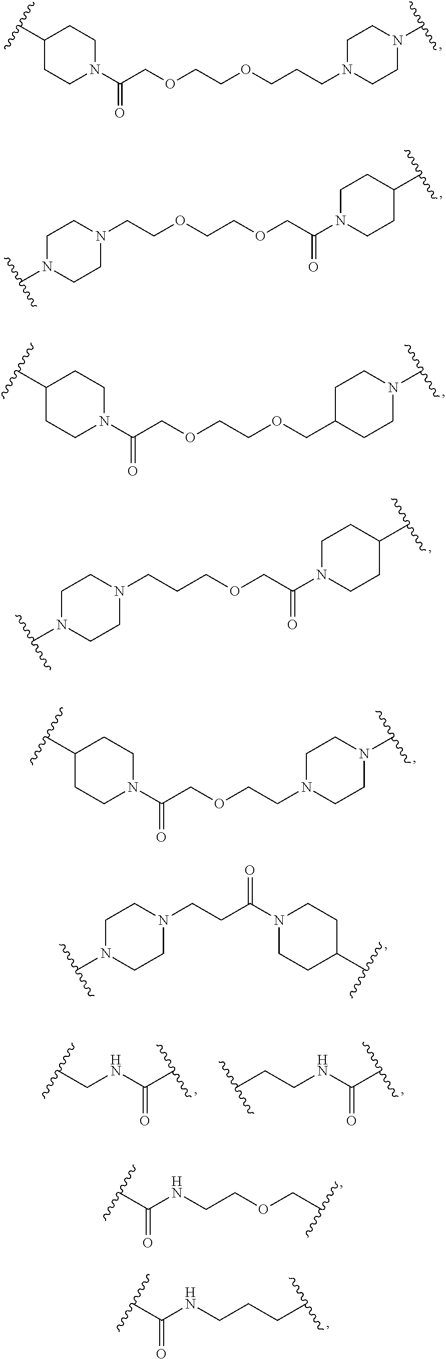

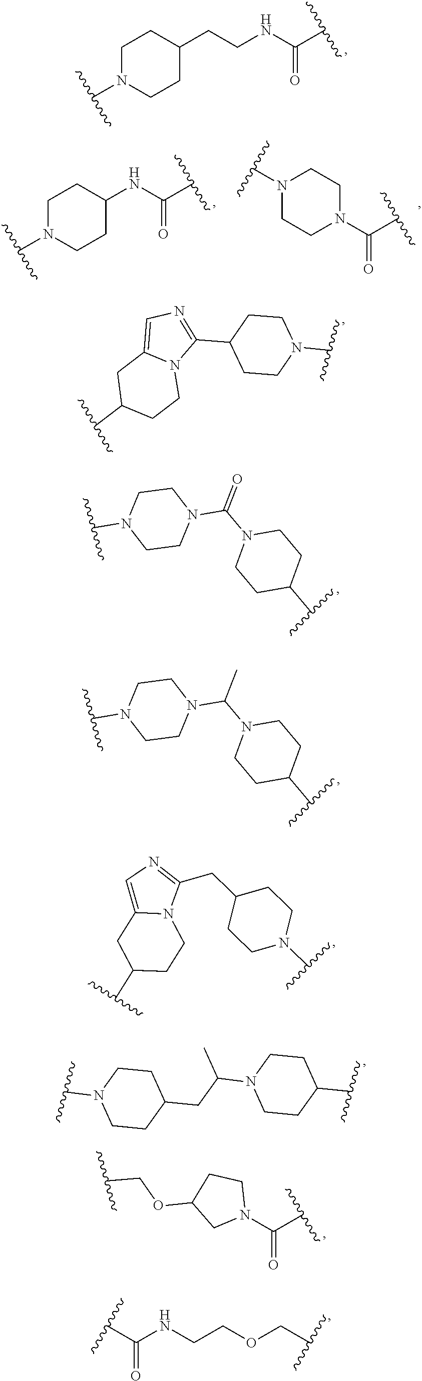

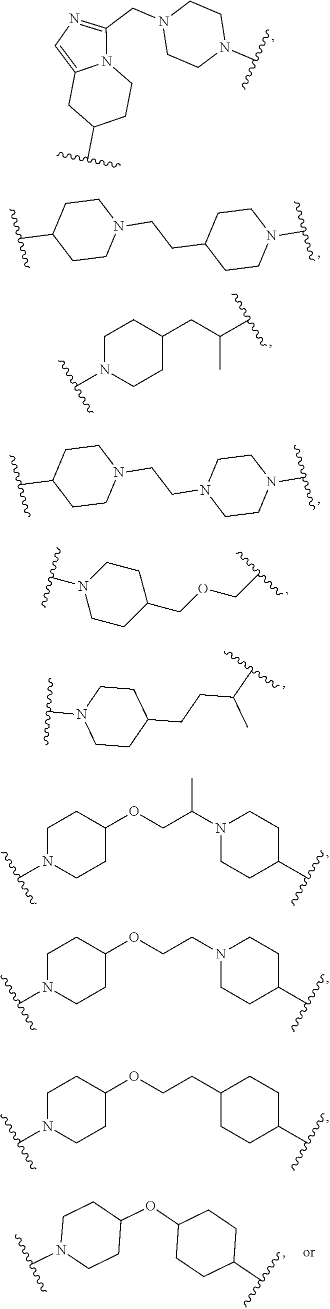

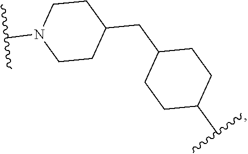 [SMILES: *C1CCC(CC2CCN(*)CC2)CC1]

or a pharmaceutically acceptable salt or solvate thereof.

In some embodiments, SB-CBP/p300 has the structure of Formula (IIIb), or a pharmaceutically acceptable salt or solvate thereof:

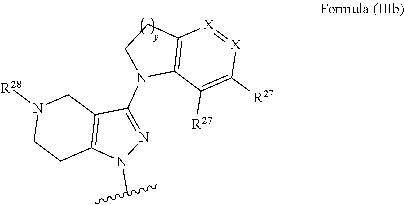

wherein:
each X is independently —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, substituted or unsubstituted C₁-C₆cycloalkyl, or substituted or unsubstituted 3- to 8-membered heterocycloalkyl, —CN, —NO₂, —OH, —OR^{a}, OC(═O)R^{a}, —OC(═O)N(R^{b})₂, —OC(═O)OR^{b}, —OC(═O)NR^{b}, —SH, —SR^{a}, —S(═O)R^{a}, —S(═O)₂R^{b}, —S(═O)₂OR^{a}, —S(═O)₂N(R^{b})₂, —N(R^{b})₂, —NR^{b} C(═O)NR^{b}, —NR^{b}C(═O)R^{a}, —NR^{b}C(═O)OR^{b}, —NR^{b}S(═O)₂R^{b}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
y is 1 or 2;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl; and
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIIb), or a pharmaceutically acceptable salt or solvate thereof, wherein:
each X is independently —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
y is 1 or 2;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl; and
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIIc), or a pharmaceutically acceptable salt or solvate thereof:

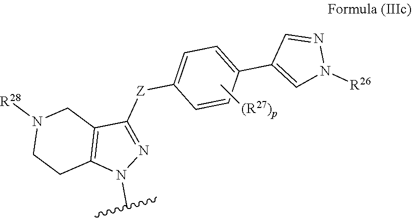

wherein:
R⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{b}, —OC(═O)NR^{b}, —SH, —SR^{a}, —S(═O)R^{a}, —S(═O)₂R^{b}, —S(═O)₂OR^{a}, —S(═O)₂N(R^{b})₂, —N(R^{b})₂, —NR^{b}C(═O)NR^{b}, —NR^{b}C(═O)R^{a}, —NR^{b}C(═O)OR^{b}, —NR^{b}S(═O)₂R^{b}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
Z is —NR^{c}—, —O—, or —S—;
R^{c} is hydrogen or substituted or unsubstituted C₁-C₆alkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl; and
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIIc), or a pharmaceutically acceptable salt or solvate thereof, wherein:
R²⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{a}, or —C(═O)N(R^{b})₂;
Z is —NR^{c}—, —O—, or —S—;
R^{c} is hydrogen or substituted or unsubstituted C₁-C₆alkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIId-1) or (IIId-2), or a pharmaceutically acceptable salt or solvate thereof:

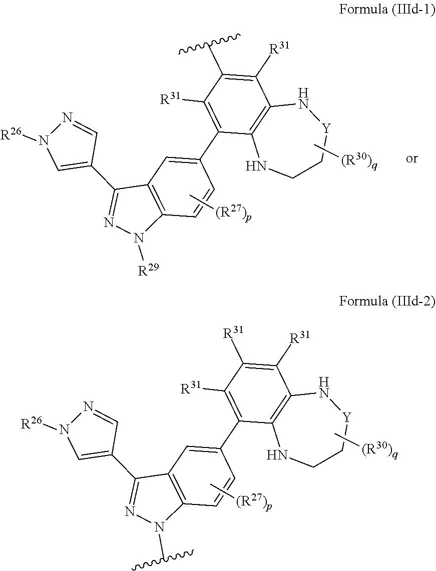

wherein:
R⁶ is hydrogen, substituted or unsubstituted C₁-C₆alkyl, substituted or unsubstituted C₁-C₆heteroalkyl, substituted or unsubstituted C₃-C₈cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{b}, —OC(═O)NR^{b}, —SH, —SR^{a}, —S(═O)R^{b}, —S(═O)₂R^{b}, —S(═O)₂OR^{a}, —S(═O)₂N(R^{b})₂, —N(R^{b})₂, —NR^{b}C(═O)NR^{b}, —NR^{b}C(═O)R^{a}, —NR^{b}C(═O)OR^{b}, —NR^{b}S(═O)₂R^{b}, —C(═O)R^{b}, —C(═O)OR^{b}, or —C(═O)N(R^{b})₂;
p is 0, 1, 2, or 3;
R⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each R¹⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{b}, —OC(═O)NR^{b}, —SH, —SR^{a}, —S(═O)R^{b}, —S(═O)₂R^{b}, —S(═O)₂OR^{a}, —S(═O)₂N(R^{b})₂, —N(R^{b})₂, —NR^{b}C(═O)NR^{b}, —NR^{b}C(═O)R^{a}, —NR^{b}C(═O)OR^{b}, —NR^{b}S(═O)₂R^{b}, —C(═O)R^{b}, —C(═O)OR^{b}, or —C(═O)N(R^{b})₂;
q is 0, 1, 2, 3, or 4;
each R¹¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{b}, —OC(═O)NR^{b}, —SH, —SR^{a}, —S(═O)R^{b}, —S(═O)₂R^{b}, —S(═O)₂OR^{a}, —S(═O)₂N(R^{b})₂, —N(R^{b})₂, —NR^{b}C(═O)NR^{b}, —NR^{b}C(═O)R^{a}, —NR^{b}C(═O)OR^{b}, —NR^{b}S(═O)₂R^{b}, —C(═O)R^{b}, —C(═O)OR^{b}, or —C(═O)N(R^{b})₂;
Y is —C(R¹⁰)₂— or C(═O);
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl; and
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIId-1) or (IIId-2), or a pharmaceutically acceptable salt or solvate thereof, wherein:
R²⁶ is hydrogen, substituted or unsubstituted C₁-C₆alkyl, substituted or unsubstituted C₁-C₆heteroalkyl, substituted or unsubstituted C₃-C₈cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
each R³¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)NR^{b}, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
Y is —C(R³⁰)₂— or C(═O);
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIIe), or a pharmaceutically acceptable salt or solvate thereof:

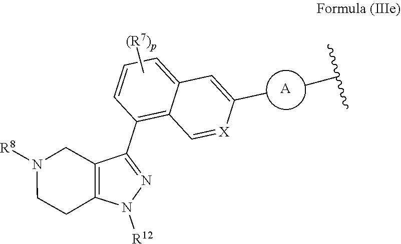

wherein:
X is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{b}, —OC(═O)NR^{b}, —SH, —SR^{a}, —S(═O)R^{b}, —S(═O)₂R^{b}, —S(═O)₂OR^{a}, —S(═O)₂N(R^{b})₂, —N(R^{b})₂, —NR^{b}C(═O)NR^{b}, —NR^{b}C(═O)R^{a}, —NR^{b}C(═O)OR^{b}, —NR^{b}S(═O)₂R^{b}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
R¹² is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
Ring A is absent or substituted or unsubstituted 3- to 8-membered heterocycloalkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has the structure of Formula (IIIe), or a pharmaceutically acceptable salt or solvate thereof, wherein:
X is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
R³² is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
Ring A is absent or substituted or unsubstituted C₃-C₈cycloalkyl, substituted or unsubstituted 3- to 8-membered heterocycloalkyl, substituted or unsubstituted aryl, or substituted or unsubstituted heteroaryl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

In some embodiments, SB-CBP/p300 has one of the following structures:

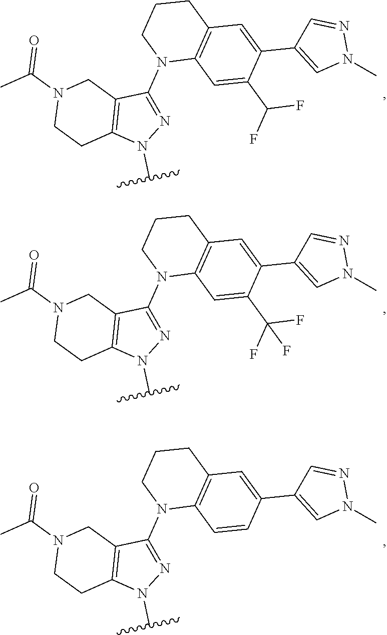

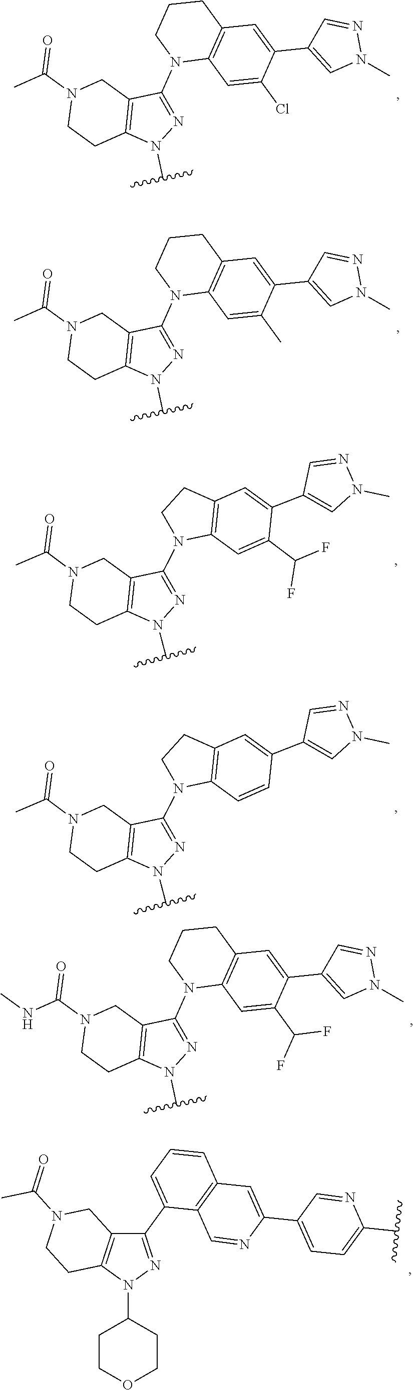

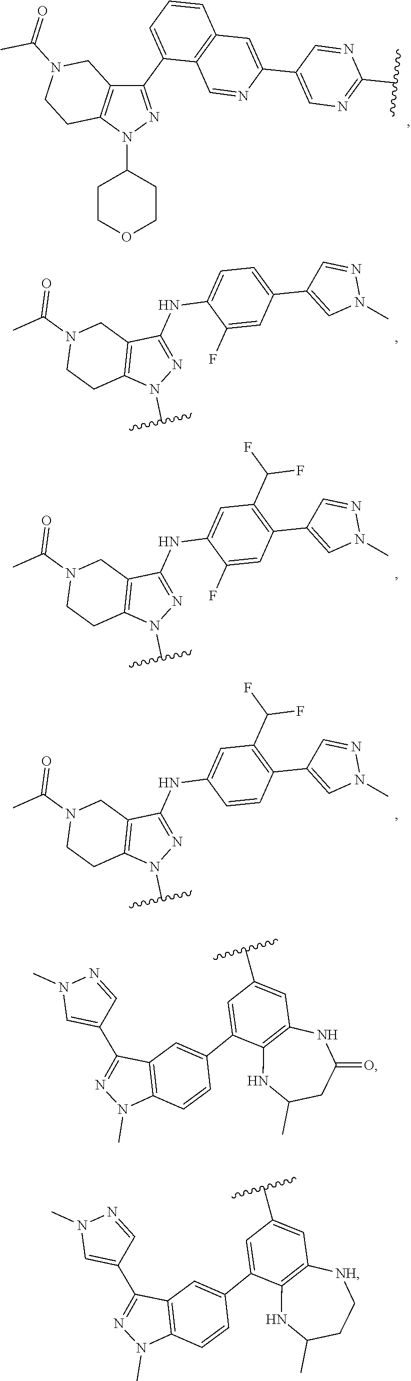

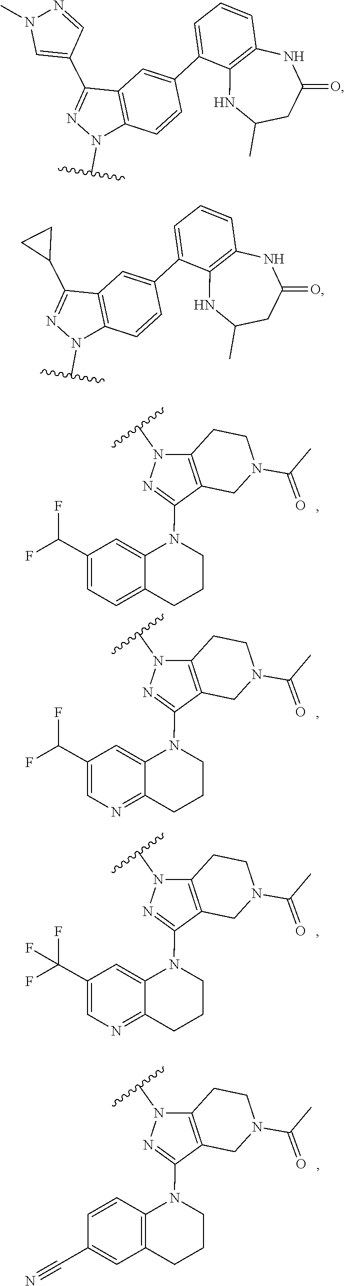

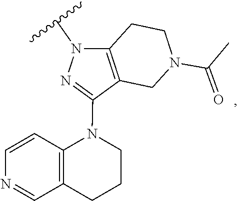 [SMILES: *n1nc(N2CCCc3cnccc32)c2c1CCN(C(C)=O)C2]

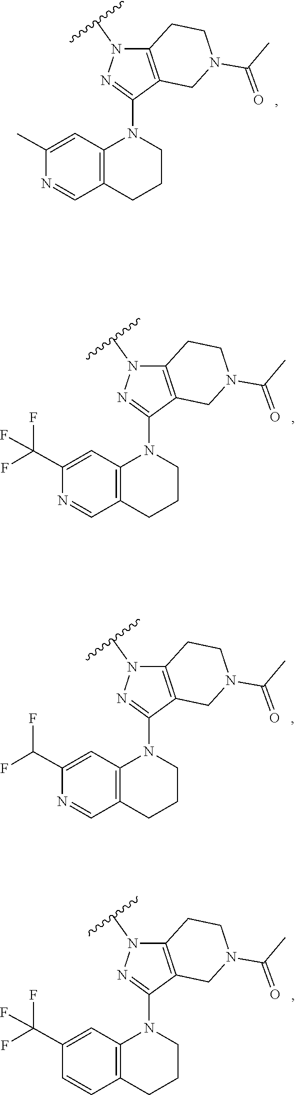 [SMILES: *n1nc(N2CCCc3cnc(C)cc32)c2c1CCN(C(C)=O)C2 ; *n1nc(N2CCCc3cnc(C(F)(F)F)cc32)c2c1CCN(C(C)=O)C2 ; *n1nc(N2CCCc3cnc(C(F)F)cc32)c2c1CCN(C(C)=O)C2 ; *n1nc(N2CCCc3ccc(C(F)(F)F)cc32)c2c1CCN(C(C)=O)C2]

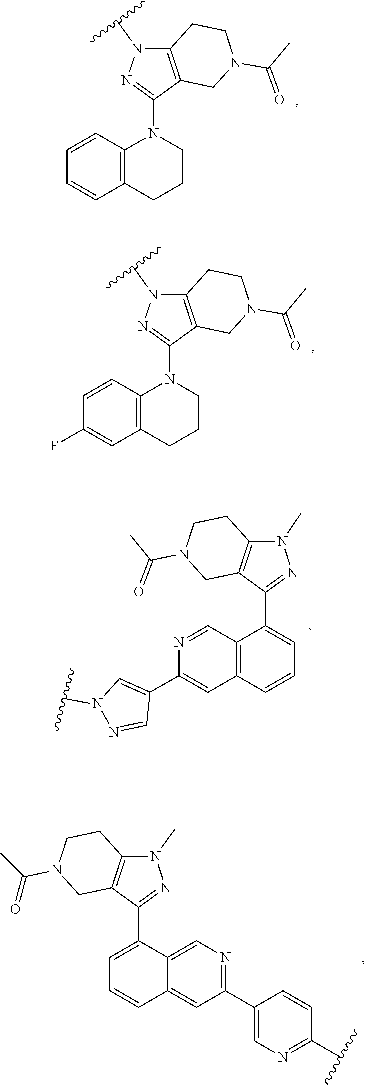

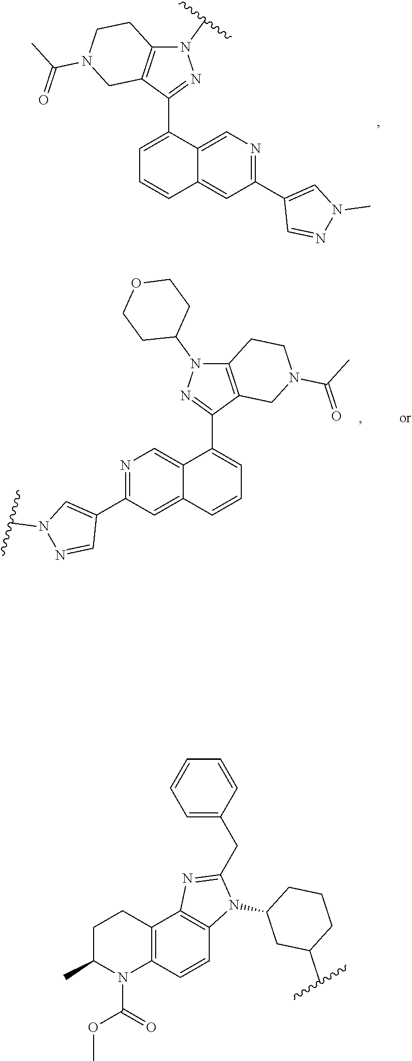

or a pharmaceutically acceptable salt or solvate thereof.

In some embodiments, SB-CBP/p300 has one of the following structures:

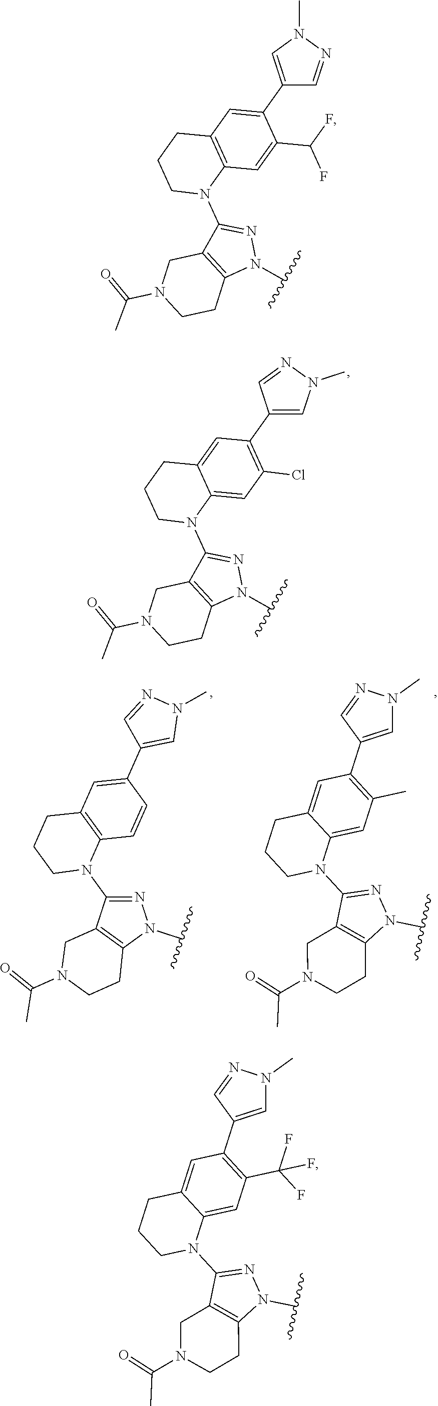

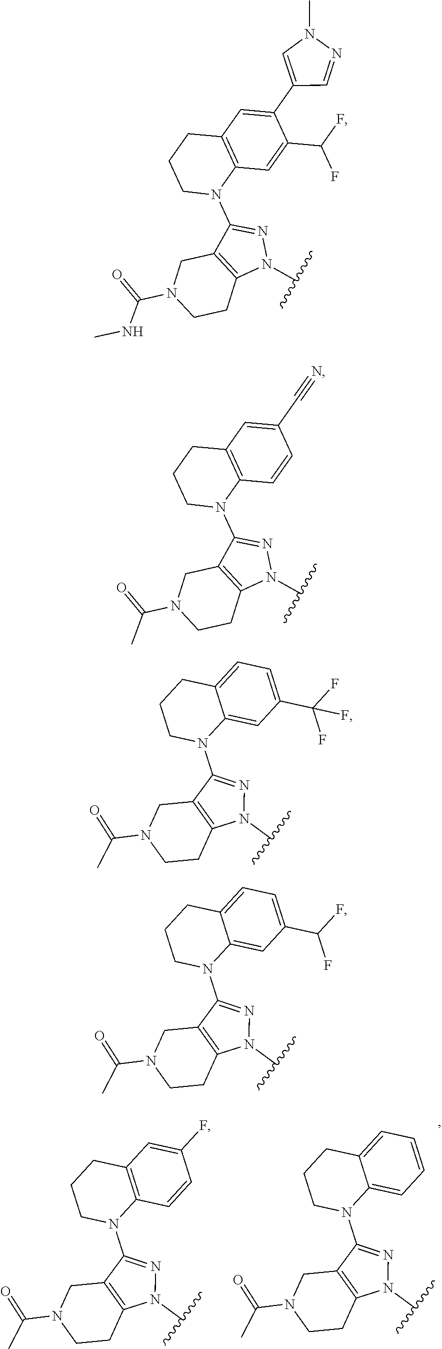

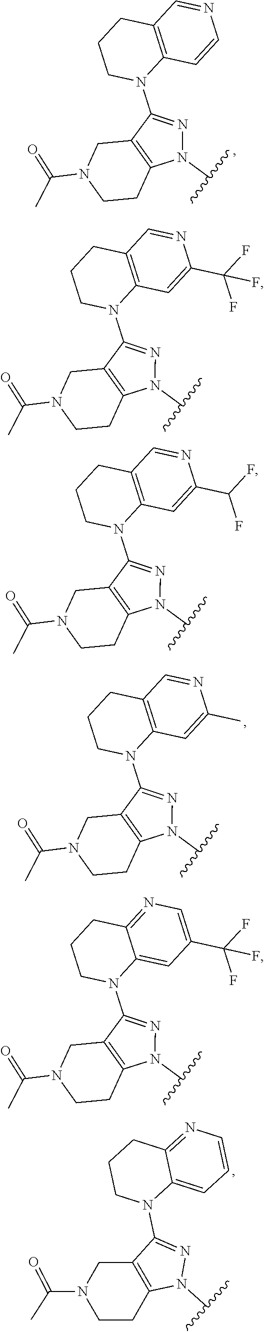

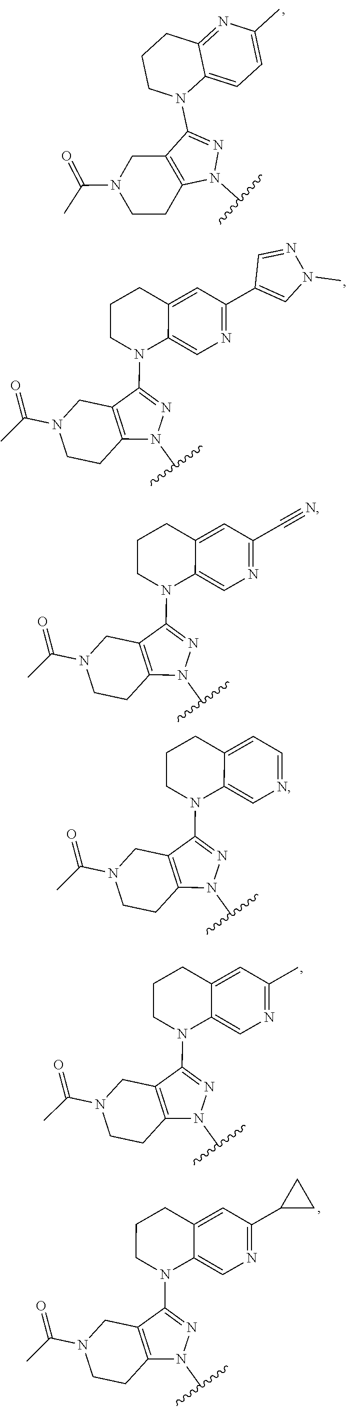

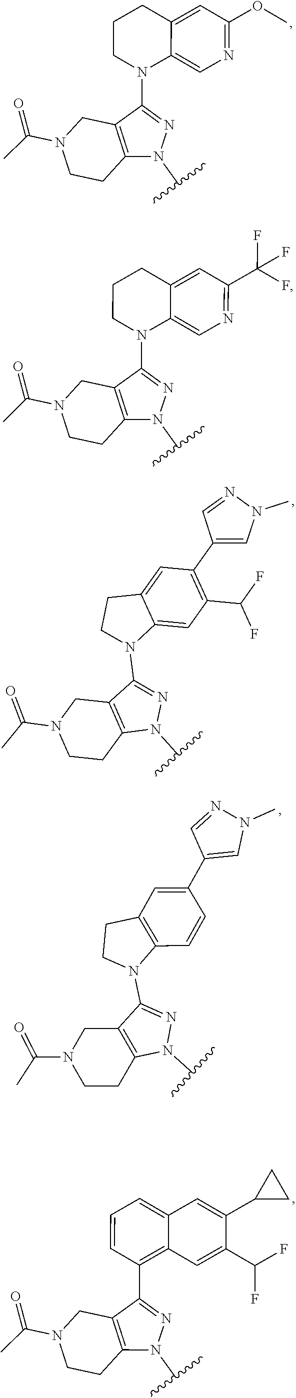

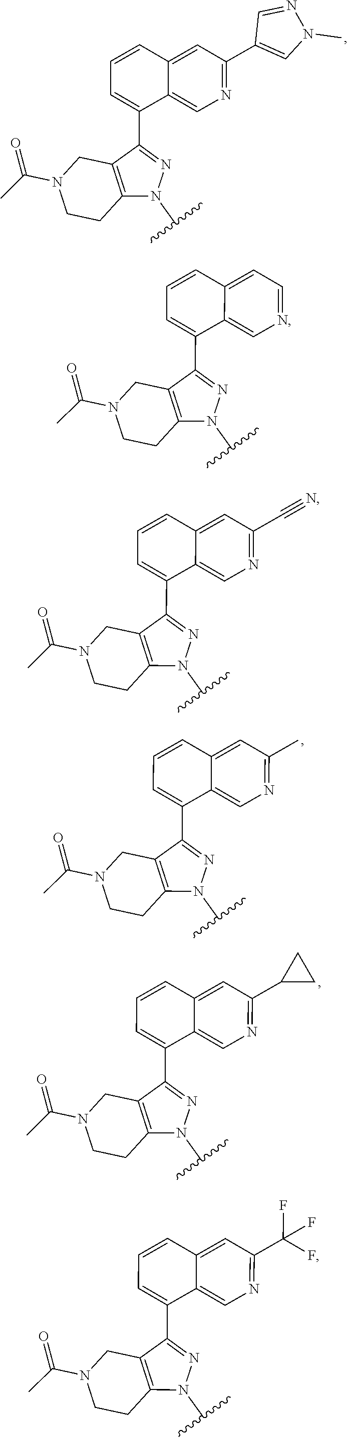

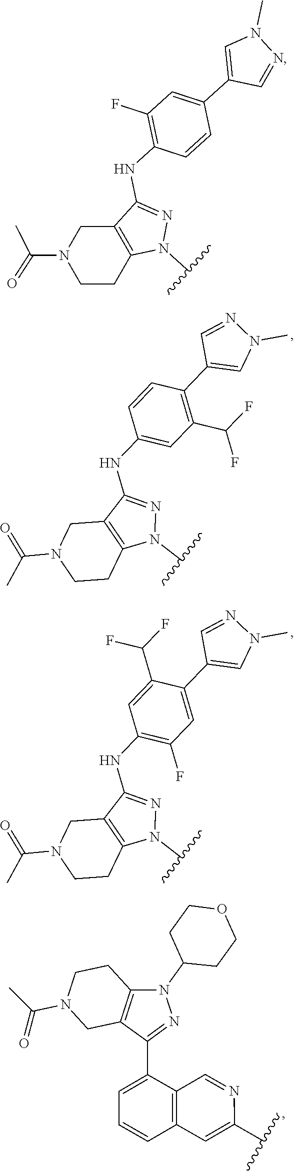

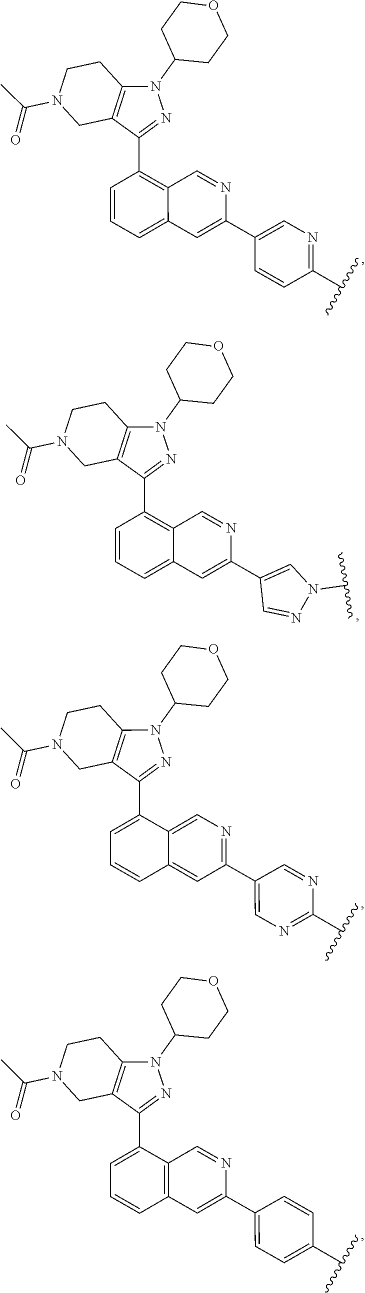 [SMILES: *c1ccc(-c2cc3cccc(-c4nn(C5CCOCC5)c5c4CN(C(C)=O)CC5)c3cn2)cn1 ; *n1cc(-c2cc3cccc(-c4nn(C5CCOCC5)c5c4CN(C(C)=O)CC5)c3cn2)cn1 ; *c1ncc(-c2cc3cccc(-c4nn(C5CCOCC5)c5c4CN(C(C)=O)CC5)c3cn2)cn1 ; *c1ccc(-c2cc3cccc(-c4nn(C5CCOCC5)c5c4CN(C(C)=O)CC5)c3cn2)cc1]

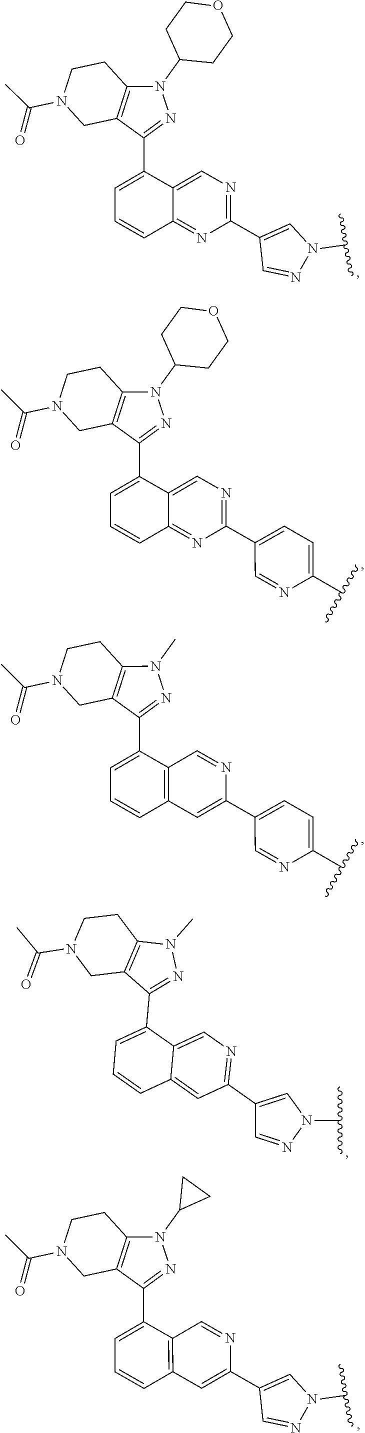 [SMILES: *n1cc(-c2ncc3c(-c4nn(C5CCOCC5)c5c4CN(C(C)=O)CC5)cccc3n2)cn1 ; *c1ccc(-c2ncc3c(-c4nn(C5CCOCC5)c5c4CN(C(C)=O)CC5)cccc3n2)cn1 ; *c1ccc(-c2cc3cccc(-c4nn(C)c5c4CN(C(C)=O)CC5)c3cn2)cn1 ; *n1cc(-c2cc3cccc(-c4nn(C)c5c4CN(C(C)=O)CC5)c3cn2)cn1 ; *n1cc(-c2cc3cccc(-c4nn(C5CC5)c5c4CN(C(C)=O)CC5)c3cn2)cn1]

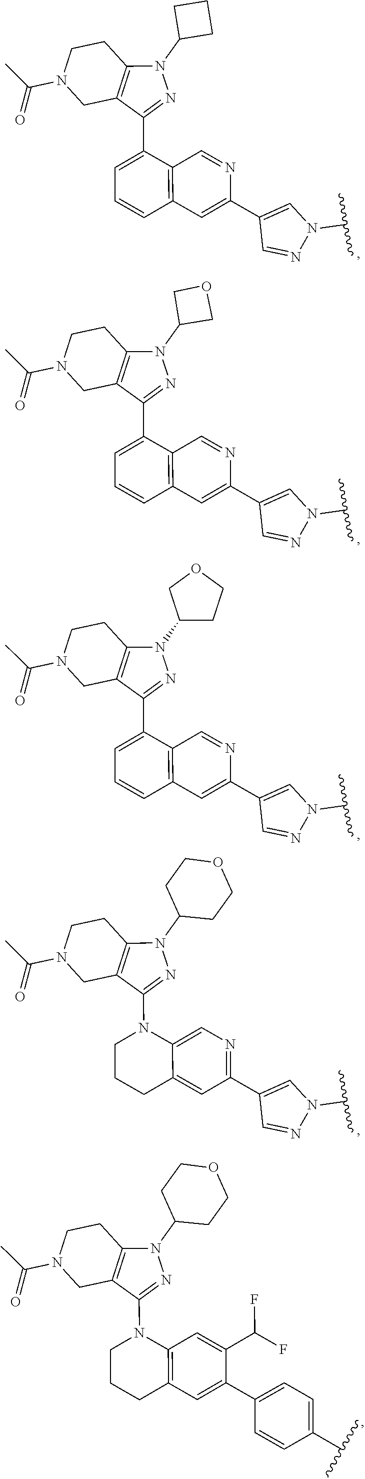 [SMILES: *n1cc(-c2cc3cccc(-c4nn(C5CCC5)c5c4CN(C(C)=O)CC5)c3cn2)cn1 ; *n1cc(-c2cc3cccc(-c4nn(C5COC5)c5c4CN(C(C)=O)CC5)c3cn2)cn1 ; *n1cc(-c2cc3cccc(-c4nn([C@H]5CCOC5)c5c4CN(C(C)=O)CC5)c3cn2)cn1 ; *n1cc(-c2cc3c(cn2)N(c2nn(C4CCOCC4)c4c2CN(C(C)=O)CC4)CCC3)cn1 ; *c1ccc(-c2cc3c(cc2C(F)F)N(c2nn(C4CCOCC4)c4c2CN(C(C)=O)CC4)CCC3)cc1]

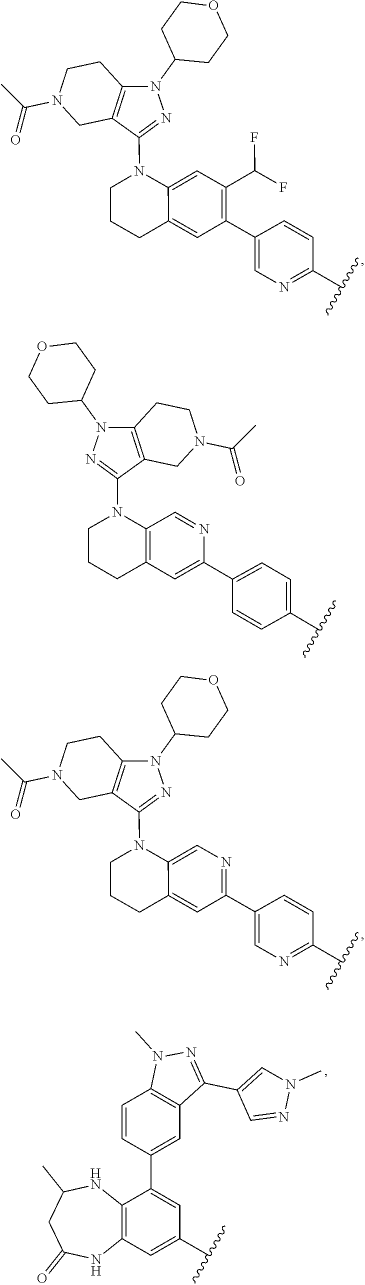

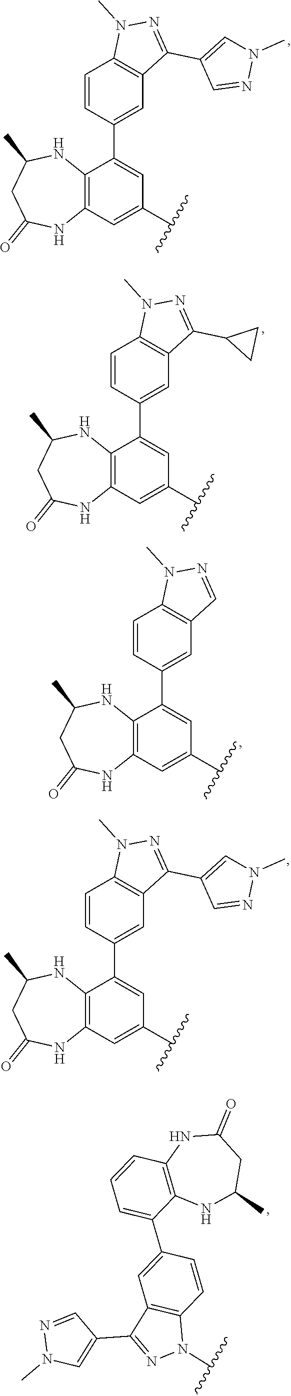

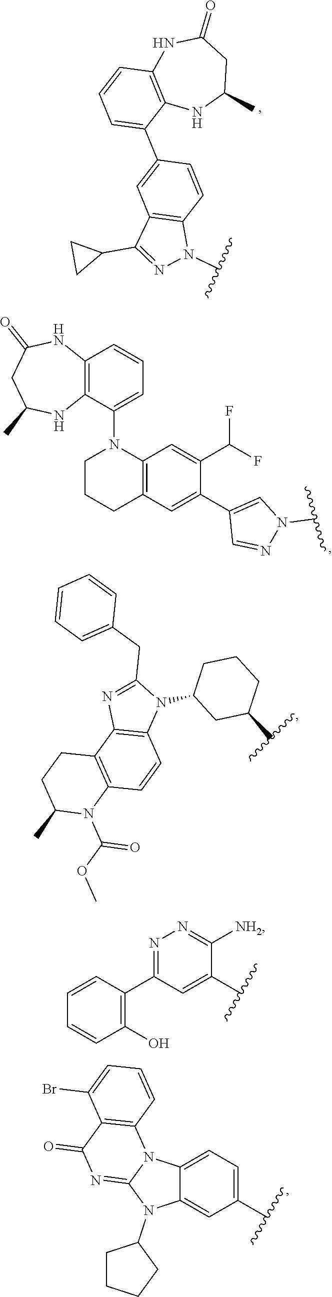

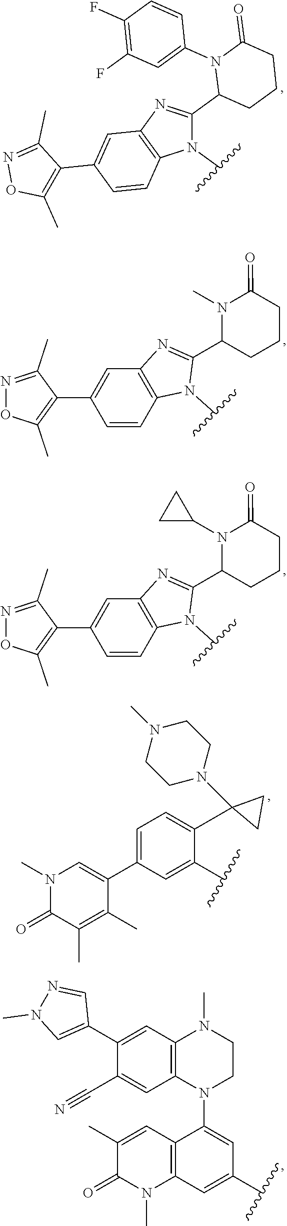

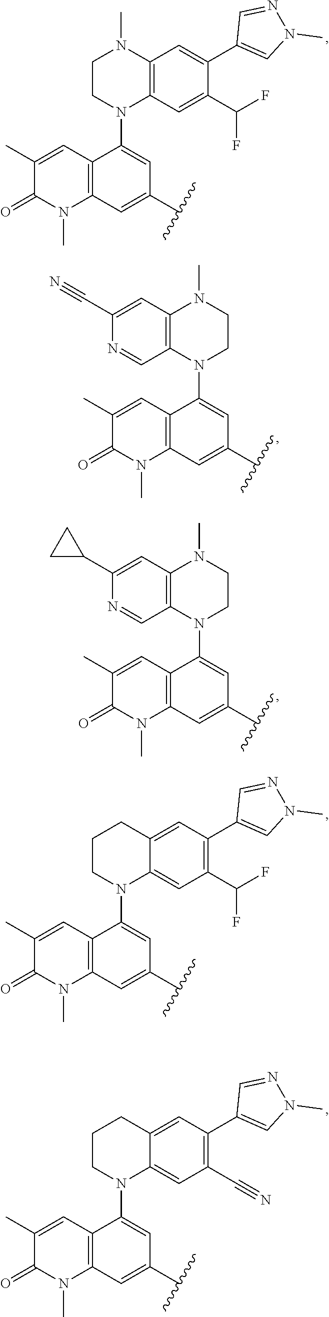

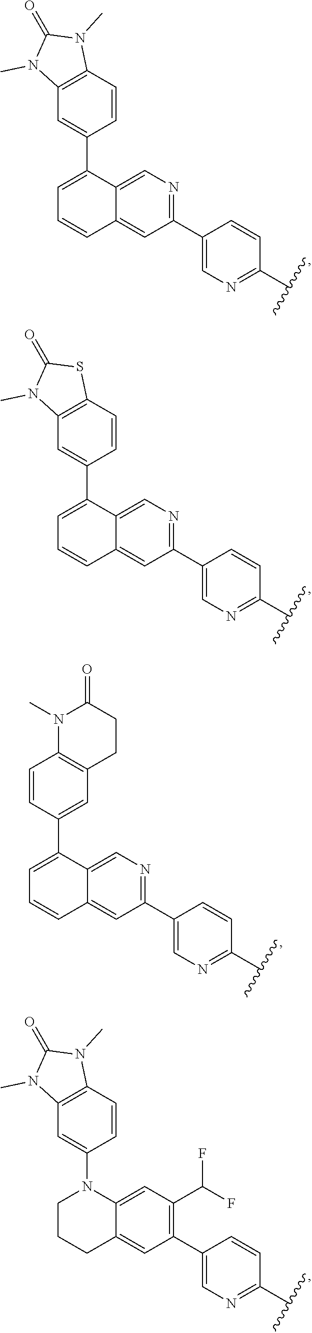 [SMILES: *c1ccc(-c2cc3cccc(-c4ccc5c(c4)n(C)c(=O)n5C)c3cn2)cn1 ; *c1ccc(-c2cc3cccc(-c4ccc5sc(=O)n(C)c5c4)c3cn2)cn1 ; *c1ccc(-c2cc3cccc(-c4ccc5c(c4)CCC(=O)N5C)c3cn2)cn1 ; *c1ccc(-c2cc3c(cc2C(F)F)N(c2ccc4c(c2)n(C)c(=O)n4C)CCC3)cn1]

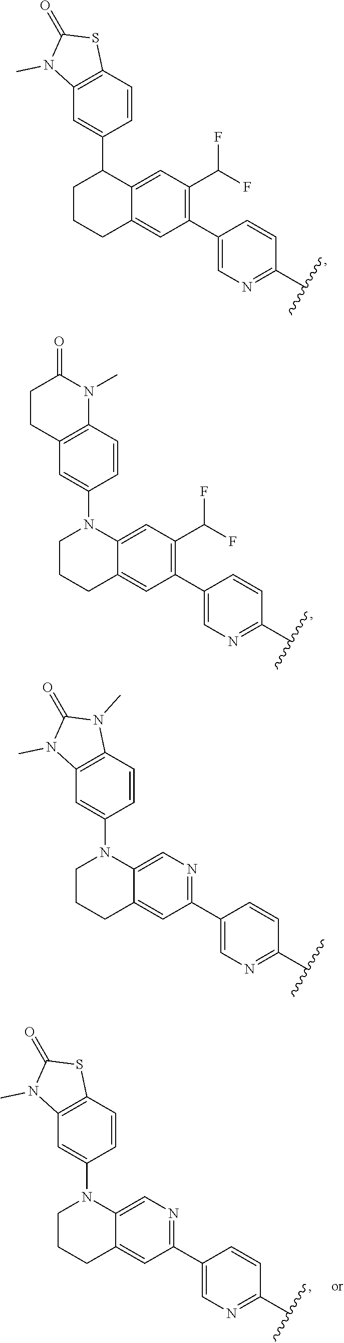 [SMILES: *c1ccc(-c2cc3c(cc2C(F)F)C(c2ccc4sc(=O)n(C)c4c2)CCC3)cn1 ; *c1ccc(-c2cc3c(cc2C(F)F)N(c2ccc4c(c2)CCC(=O)N4C)CCC3)cn1 ; *c1ccc(-c2cc3c(cn2)N(c2ccc4c(c2)n(C)c(=O)n4C)CCC3)cn1 ; *c1ccc(-c2cc3c(cn2)N(c2ccc4sc(=O)n(C)c4c2)CCC3)cn1]

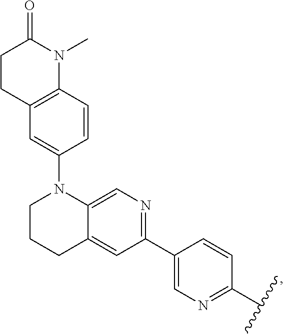 [SMILES: *c1ccc(-c2cc3c(cn2)N(c2ccc4c(c2)CCC(=O)N4C)CCC3)cn1]

or a pharmaceutically acceptable salt or solvate thereof.

In some embodiments, SB-CBP/p300 has one of the following structures:

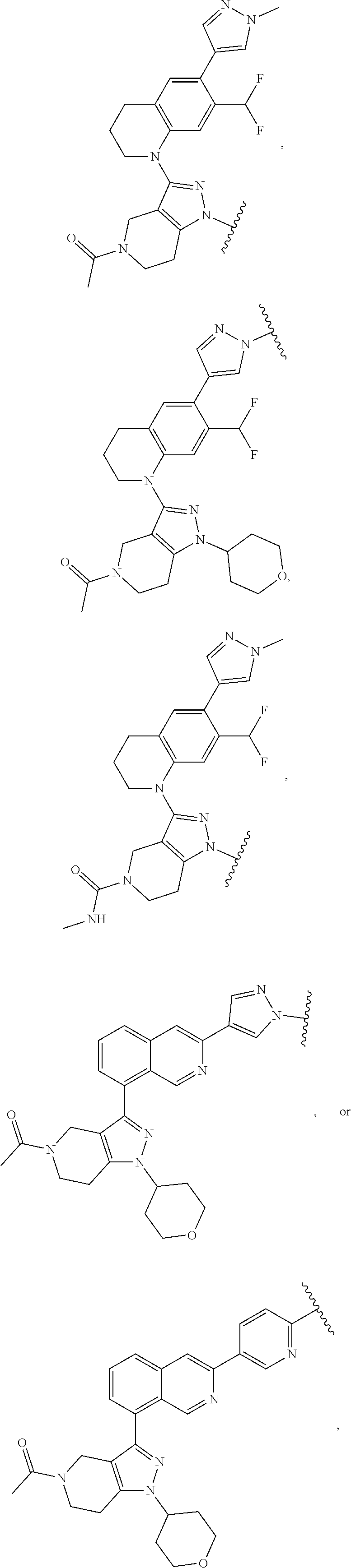

or a pharmaceutically acceptable salt or solvate thereof.

Embodiment 1. A heterobifunctional conditional inhibitor compound of Formula (I):

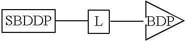

wherein:
SBDDP is a silent binder of a disease-dependent protein (DDP);
L is an optional linker; and
BDP is a binder of a disease protein (DP);
wherein DDP and DP are both expressed in a cell of interest (COI) and the relative abundance of the DP in the COI is greater than the relative abundance of the DDP in the COI; or
wherein the COI is a diseased cell, and the DP is overexpressed, overactive, or both overexpressed and overactive, or amplified in the diseased cell as compared to when the COI is a non-diseased cell.

Embodiment 2. The compound of any one of embodiments 1-4, wherein the activity of the DDP is reduced or inhibited by the compound of Formula (I) when the DDP and DP are both expressed in the same COI and the relative abundance of the DP in the COI is greater than the relative abundance of the DDP in the COI.

Embodiment 3. The compound of embodiment 1 or 2, wherein DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3.

Embodiment 4. The compound of embodiment 1 or 2, wherein DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

Embodiment 5. The compound of embodiment 1 or 2, wherein DDP is a human bromodomain-containing protein (BRD).

Embodiment 6. The compound of embodiment 1 or 2, wherein DDP is a human bromodomain-containing protein that is a Group Ia BRD, Group Ib BRD, Group II BRD, Group IIIa BRD, Group IIIb BRD, Group IIII BRD, Group IV BRD, Group V BRD, Group VI BRD, Group VI BRD, Group VIII BRD, or group IX BRD.

Embodiment 7. The compound of embodiment 6, wherein:
the Group Ia BRD is selected from PCAF, GCN5L2, p300/CBP, TAF1 and TAF1L;
the Group Ib BRD is selected from BRPF1A/B (BR140), BRPF2 (BRD1) BRPF3 and BRD8 (SMAP);
the Group II BRD is selected from histone methyltransferases ASH1L and MLL;
the Group IIIa BRD is selected from chromatin remodeling factors SMARCA2 (BRM), SMARCA4 (BRG1), BRD7, BRD9 and PBRM1 (polybromo);
the Group IIIb BRD is selected from ISWI family chromatin remodeling factors BAZ1A (ACF1), BAZ1B (WSTF, William-Beuren syndrome transcription factor), BAZ2A, BAZ2B, BPTF and CECR2;
the Group IV BRD is selected from ATPase family AAA domain-containing proteins ATAD2 and ATAD2B;
the Group V BRD is selected from BET family proteins BRD2, BRD3, BRD4 and testis-specific BRDT;
the Group VI BRD is selected from Tripartite-motif-containing (TRIM) family proteins TRIM24 (TIF1α), TRIM28 (TIF1β, KAP1), TRIM33 (TIF1γ) and TRIM66 (TIF1δ);
the Group VIII BRD is selected from speckled protein (SP) family proteins SP100, SP110, SP140 and SP140L;
the Group VIII BRD is selected from transcriptional corepressors ZMYND8 and ZMYDN11; and
the Group IX is selected from WD-repeat proteins BRWD1 (WDR9), BRWD3 and PHIP (WDR11).

Embodiment 8. The compound of embodiment 1 or 2, wherein DDP is a human bromodomain-containing protein (BRD) is:
a Group Ia BRD selected from PCAF, GCN5L2, p300/CBP, TAF1 and TAF1L;
a Group Ib BRD selected from BRPF1A/B (BR140), BRPF2 (BRD1) BRPF3 and BRD8 (SMAP);
a Group II BRD selected from histone methyltransferases ASH1L and MLL;
a Group IIIa BRD selected from chromatin remodeling factors SMARCA2 (BRM), SMARCA4 (BRG1), BRD7, BRD9 and PBRM1 (polybromo);
a the Group IIIb BRD selected from ISWI family chromatin remodeling factors BAZ1A (ACF1), BAZ1B (WSTF, William-Beuren syndrome transcription factor), BAZ2A, BAZ2B, BPTF and CECR2;
a Group IV BRD selected from ATPase family AAA domain-containing proteins ATAD2 and ATAD2B;
a Group VI BRD selected from Tripartite-motif-containing (TRIM) family proteins TRIM24 (TIF1α), TRIM28 (TIF1β, KAP1), TRIM33 (TIF1γ) and TRIM66 (TIF1δ);
a Group VIII BRD selected from speckled protein (SP) family proteins SP100, SP110, SP140 and SP140L;
a Group VIII BRD selected from transcriptional corepressors ZMYND8 and ZMYDN11; and
a Group IX selected from WD-repeat proteins BRWD1 (WDR9), BRWD3 and PHIP (WDR11).

Embodiment 9. A heterobifunctional compound of Formula (Ia), comprising:

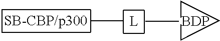

SB-CBP/p300 a silent binder of human CREB-binding protein (CBP) or human E1A-binding protein p300 (p300) (CBP/p300);
BDP is a binder of a disease protein (DP);
L is an optional linker covalently connecting SB-CBP/p300 to BDP;
wherein L is covalently attached at a position of SB-CBP/p300 that is solvent exposed when SB-CBP/p300 binds to CBP/p300, and L is covalently attached at a position of BDP that is solvent exposed when BDP binds to DP;
wherein CBP/p300 and DP are both expressed in a cell of interest (COI) and the relative abundance of the DP in the COI is greater than the relative abundance of CBP/p300 in the COI; or
wherein the COI is a diseased cell, and the DP is overexpressed, overactive, or both overexpressed and overactive, or amplified in the diseased cell as compared to when the COI is a non-diseased cell.

Embodiment 10. The compound of any one of embodiments 1-9, wherein the DP is androgen receptor (AR), estrogen receptor (ER), progesterone receptor (PR), glucocorticoid receptor (GR), mineralocorticoid receptor (MR), retinoic acid receptor-related orphan nuclear receptor γ (RORγ), angiotensin receptor, apelin receptor, bombesin receptor, bradykinin receptor, calcitonin receptor, chemokine receptor, cholecytokinin receptor, chymase, corticotropic-releasing factor receptor, galanin receptor, ghrelin receptor, glucagon receptor, glycoprotein hormone receptor, gonadotropin-releasing hormone receptor, indoleamine 2,3-dioxygenase 1 (IDO1), kisspeptin receptor, melanocortin receptor, motilin receptor, neuromedin U receptor, neuropeptide FF/AF receptor, neuropeptide S receptor, neuropeptide W/B receptor, neuropeptide Y receptor, opioid receptor, orexin receptor, parathyroid hormone receptor, prokineticin receptor, prolactin-releasing peptide receptor, QRFP receptor, relaxin family peptide receptor, somatostatin receptor, tachykinin receptor, thyrotropin-releasing hormone receptor, urotensin receptor, vasopressin, oxytocin receptor, vasoactive intestinal peptide receptor, PACAP receptor, HER1, HER2, HER3, HER4, KRAS, KRAS-G12C, HRAS, NRAS, or BCL6.

Embodiment 11. The compound of any one of embodiments 9 or 10, wherein SB-CBP/p300 binds to the bromodomain of human CREB-binding protein (CBP) or human E1A-binding protein p300 (p300) (CBP/p300).

Embodiment 12. The compound of embodiment 11, or a pharmaceutically acceptable salt or solvate thereof, wherein SB-CBP/p300 binds in the acetyl-lysine (KAc) binding site of the bromodomain of human CREB-binding protein (CBP) or human E1A-binding protein p300 (p300) (CBP/p300).

Embodiment 13. The compound of embodiment 12, or a pharmaceutically acceptable salt or solvate thereof, wherein:
the moiety comprising an acetyl-lysine mimetic makes or mimics a hydrogen bond interaction to Asn1168 in the Asn-binding pocket of the bromodomain of CBP, or makes or mimics a hydrogen bond interaction to Asn1132 in the Asn-binding pocket of the bromodomain of p300.

Embodiment 14. The compound of any one of embodiments 12-13, or a pharmaceutically acceptable salt or solvate thereof, wherein: SBDDP further comprises a moiety that interacts with Arg1173 in the bromodomain of CBP or Asn1137 in the bromodomain of p300.

Embodiment 15. The compound of any one of embodiments 12-14, or a pharmaceutically acceptable salt or solvate thereof, wherein the moiety comprising an acetyl-lysine mimetic comprises a moiety selected from pyrrolidonyl, phenyl, pyridinyl, pyridinonyl, triazolyl, pyrrolyl, isoxazolyl, pyrazolyl, quinolinyl, tetrahydroquinolinyl, isoquinolinyl, tetrahydroisoquinolinyl, quinazolonyl, quinazolinyl, dihydroquinazolinonyl, imidazo[4,5-c]quinolinyl fused to a dimethylisoxazolyl, triazolophthalazinyl, indolizinyl, benzoimidazolyl, isoxazole-indolizinyl, thienodiazepine-indolizinyl, benzodiazepine-indolizinyl, 5-isoxazolylbenzimidazolyl, 6-isoxazolylbenzimidazolyl, 7-isoxazolo-quinolinyl, diazobenzyl, triazolophthalazinyl, isoxazoloquinolinyl, 2-thiazolidinonyl, triazolopyrimidinyl, thienodiazepinyl, benzodiazepinyl, benzotriazepinyl, triazolobenzodiazepinyl, triazolothienodiazepinyl, triazolothienodiazepinyl, and isoxazole-azepinyl.

Embodiment 16. The compound of any one of embodiments 12-15, or a pharmaceutically acceptable salt or solvate thereof, wherein SB-CBP/p300 further comprises:
1) a moiety that occupies the lipophilic shelf (LPF) region of the bromodomain of CBP/p300; or
2) a moiety that occupies the BC Loop region of the bromodomain of CBP/p300; or
3) both 1) and 2);
wherein L is covalently attached to SB-CBP/p300 on:
   the acetyl-lysine mimetic moiety; or
   the moiety that occupies the lipophilic shelf (LPF) region of the bromodomain of CBP/p300, if present; or
   the moiety that occupies the BC Loop region of the bromodomain of CBP/p300, if present;
wherein L is covalently attached to SB-CBP/p300 at a position that does not interfere with the binding of the acetyl-lysine mimetic moiety in the acetylated lysine (KAc) binding site of CBP/p300.

Embodiment 17. The compound of any one of embodiments 12-16, or a pharmaceutically acceptable salt or solvate thereof, wherein the moiety comprising an acetyl-lysine mimetic comprises a moiety selected from:

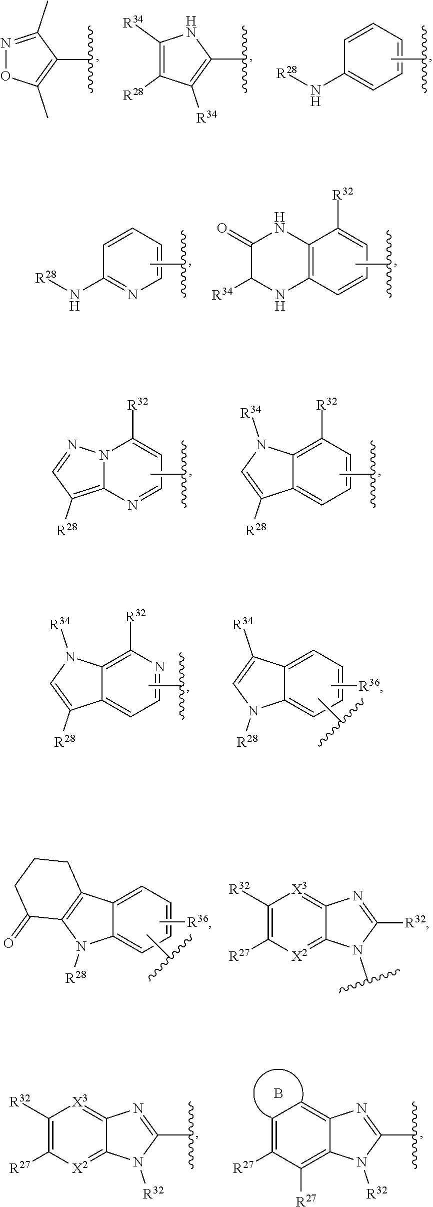

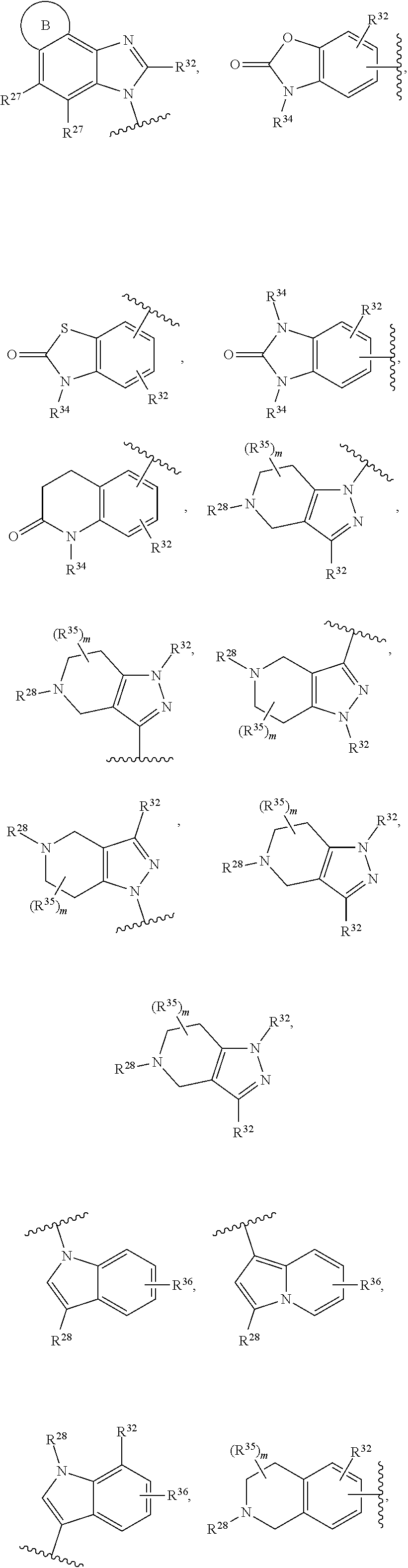

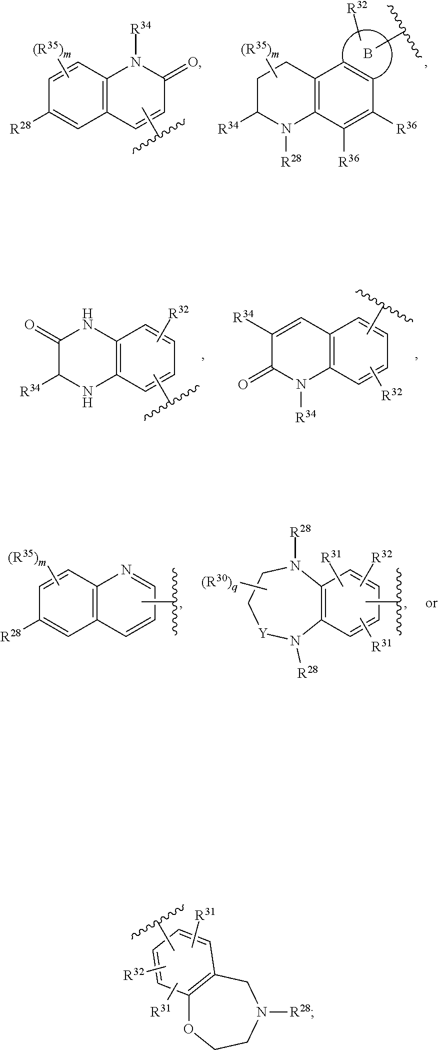

each R³² is independently an optional moiety that occupies the lipophilic shelf (LPF) region of the bromodomain of CBP/p300;
or each R³² is independently an optional moiety that occupies the BC Loop region of the bromodomain of CBP/p300;
[symbol] is the point of attachment to L that covalently connects SB-CBP/p300 to BDP;
or R³² comprises [symbol] and L that covalently connects SB-CBP/p300 to BDP is attached to R³²;
each R²⁸ is independently hydrogen, substituted or unsubstituted C₁-C₆alkyl, substituted or unsubstituted C₁-C₆fluoroalkyl, —C(═O)R^{b}, or —C(═O)N(R^{b})₂;
each R³⁴ is independently hydrogen or substituted or unsubstituted C₁-C₆alkyl;
each R³⁵ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl, —CN, —OH, —OR^{a}, or —N(R^{b})₂;
m is 0, 1, 2, 3, or 4;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
Y is —C(R³⁰)₂— or C(═O);
each X³ is independently CR²⁷ or N;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
each R³¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)NR^{b}, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
each R³⁶ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR^{a}, or —N(R^{b})₂;
Ring B is a fused substituted or unsubstituted 5 or 6 membered heterocycloalkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 18. The compound of embodiment 12-17, or a pharmaceutically acceptable salt or solvate thereof, wherein the moiety that occupies the lipophilic shelf (LPF) region of the bromodomain of CBP/p300 is R³², wherein:
R³² is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₂cycloalkyl, substituted or unsubstituted 3- to 12-membered heterocycloalkyl, substituted or unsubstituted aryl, or substituted or unsubstituted heteroaryl;
or R³² is

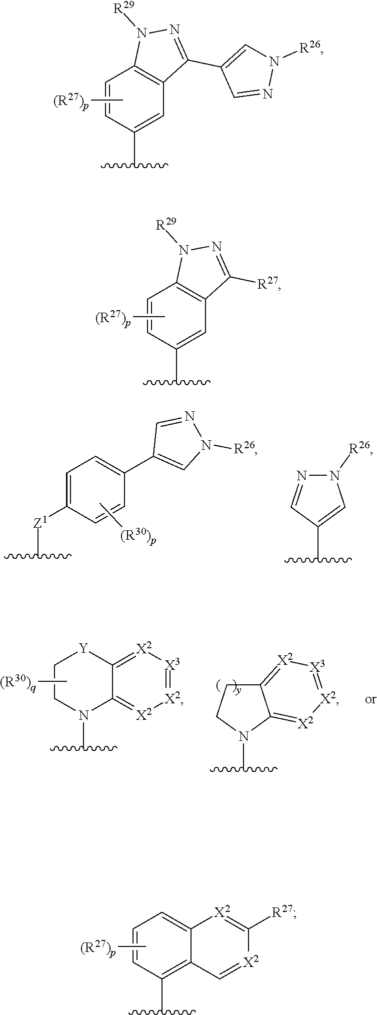

y is 1 or 2;
Z¹ is —NR^{c}—, —O—, or —S—;
R^{c} is hydrogen or substituted or unsubstituted C₁-C₆alkyl;
R²⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each X² is independently —CR³⁰— or —N—;
each X³ is independently —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen or substituted or unsubstituted C₁-C₆alkyl;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
Y is —C(R³⁰)₂— or N(R²⁸)—;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
or R³² is -L-C;
L is substituted or unsubstituted C₁-C₆alkyl or substituted or unsubstituted C₁-C₆heteroalkyl;
C is substituted or unsubstituted C₃-C₈cycloalkyl, substituted or unsubstituted 3- to 8-membered heterocycloalkyl, substituted or unsubstituted aryl, or substituted or unsubstituted heteroaryl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl; and
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 19. The compound of any one of embodiments 12-18, or a pharmaceutically acceptable salt or solvate thereof, wherein the moiety that occupies the BC Loop region of the bromodomain of CBP/p300 is R³², wherein:
R³² is

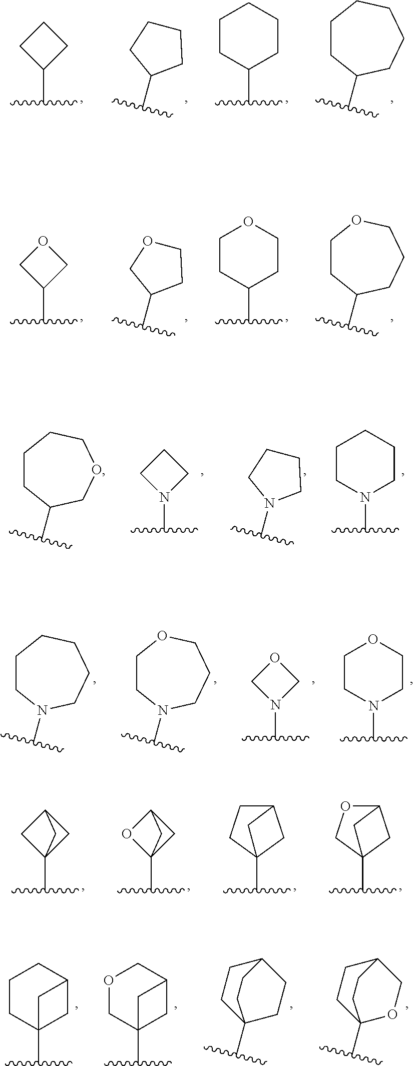

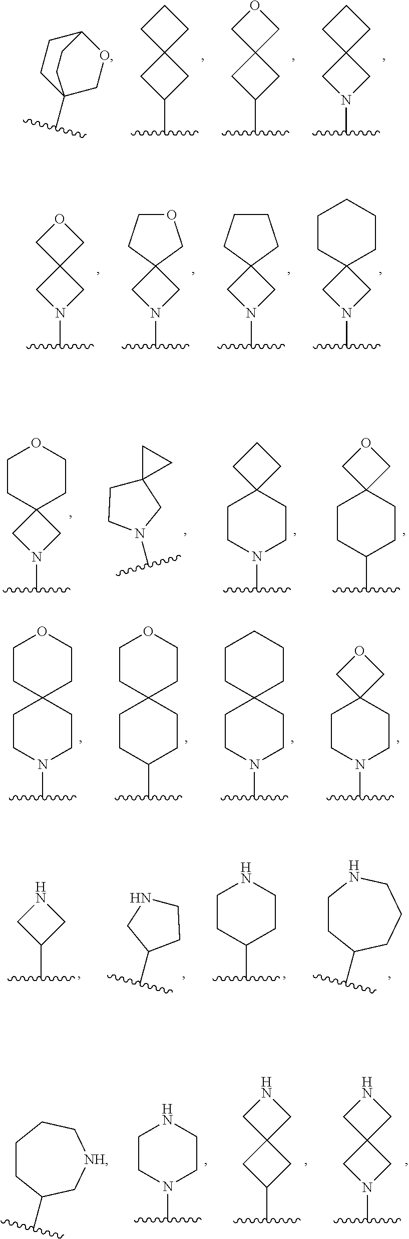

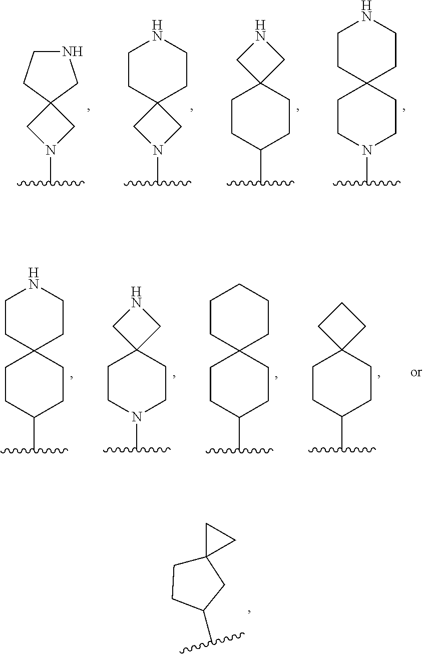

or each of which is substituted or unsubstituted.

Embodiment 20. The compound of any one of embodiments 12-19, or a pharmaceutically acceptable salt or solvate thereof, wherein the moiety comprising an acetyl-lysine mimetic comprises

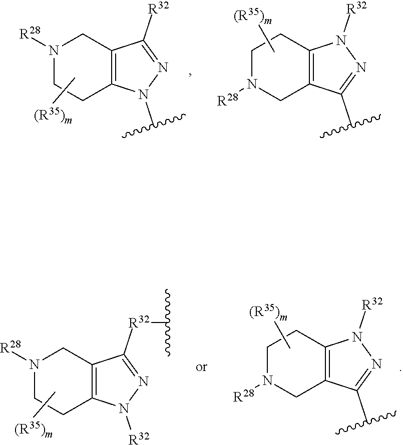

Embodiment 21. The compound of any one of embodiments 12-19, or a pharmaceutically acceptable salt or solvate thereof, wherein the moiety comprising an acetyl-lysine mimetic comprises

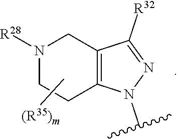

Embodiment 22. The compound of embodiment 21, or a pharmaceutically acceptable salt or solvate thereof, wherein:
R³² is

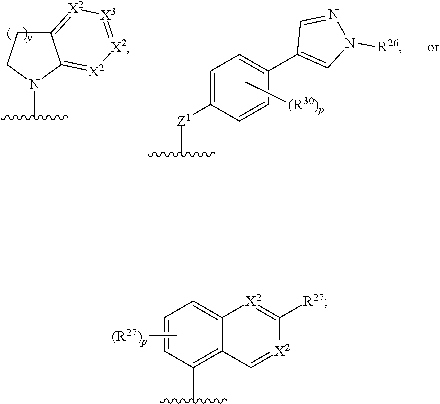

R²⁷ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, substituted or unsubstituted heteroaryl, —CN, —OH, or —OR^{a}; and
each R³⁰ is independently hydrogen, substituted or unsubstituted C₁-C₆alkyl, substituted or unsubstituted C₁-C₆fluoroalkyl, substituted or unsubstituted C₁-C₆heteroalkyl, —CN, —OH, or —OR^{a}; and
each R^{a} is independently substituted or unsubstituted C₁-C₆alkyl.

Embodiment 23. The compound of embodiment 22, or a pharmaceutically acceptable salt or solvate thereof, wherein:
each R²⁷ is independently hydrogen, —CH₃, —CH₂CH₃, —F, —CHF₂, —CF₃, —CN, —OH, —OCH₃, cyclopropyl, cyclobutyl, oxetanyl, tetrahydrofuranyl, tetrahydropyranyl, phenyl, pyrazolyl, 1-methyl pyrazolyl, pyridinyl, or pyrimidinyl.

Embodiment 24. The compound of embodiment 21, or a pharmaceutically acceptable salt or solvate thereof, wherein:
R³² is

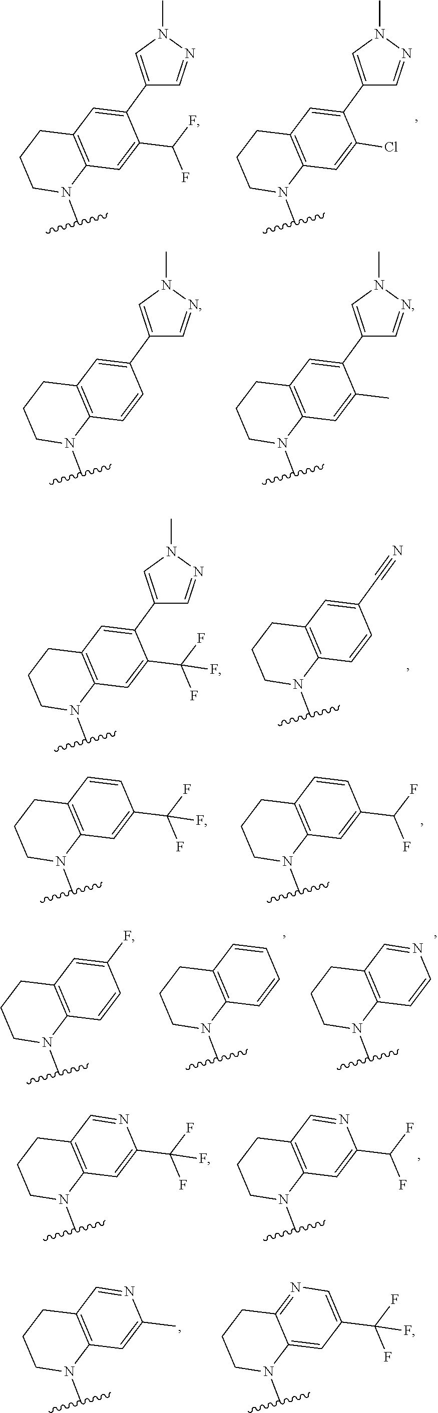

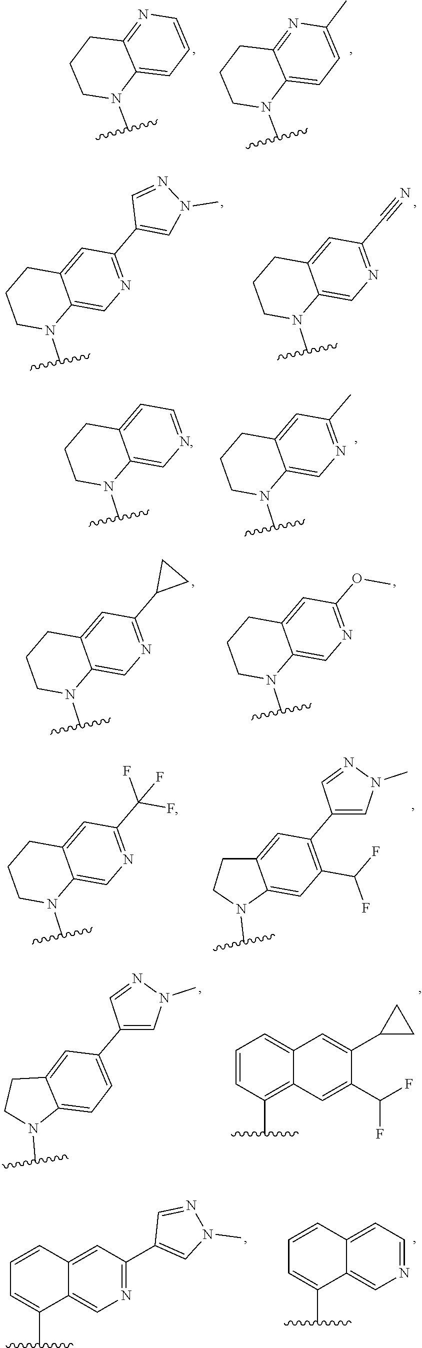

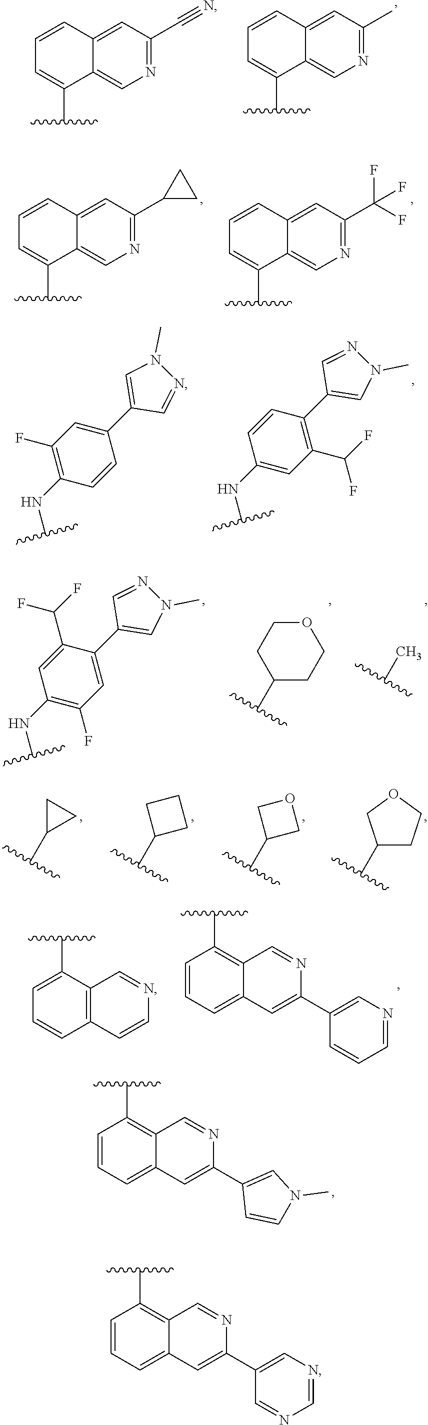

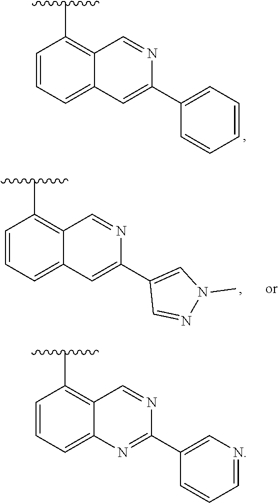 [SMILES: *c1cccc2cc(-c3ccccc3)ncc12 ; *c1cccc2cc(-c3cnn(C)c3)ncc12 ; *c1cccc2nc(-c3cccnc3)ncc12]

Embodiment 25. The compound of any one of embodiments 12-19, or a pharmaceutically acceptable salt or solvate thereof, wherein the moiety comprising an acetyl-lysine mimetic comprises

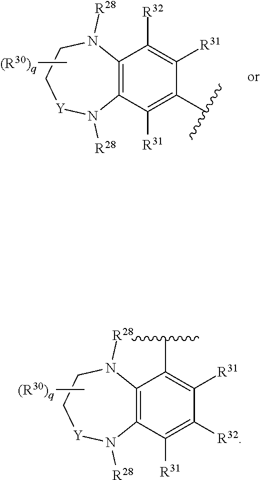

Embodiment 26. The compound of embodiment 25, or a pharmaceutically acceptable salt or solvate thereof, wherein the moiety comprising an acetyl-lysine mimetic comprises

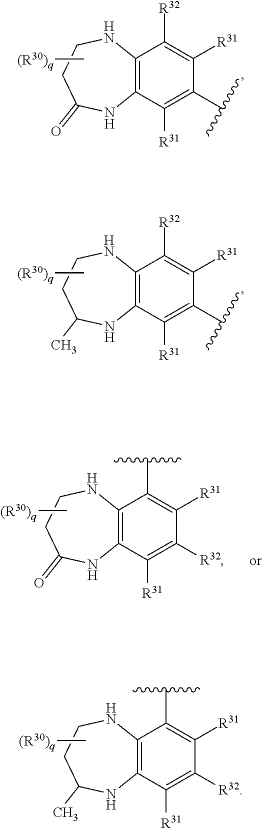

Embodiment 27. The compound of embodiment 25, or a pharmaceutically acceptable salt or solvate thereof, wherein the moiety comprising an acetyl-lysine mimetic comprises

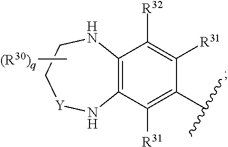

wherein:
each R²⁸ is independently hydrogen or substituted or unsubstituted C₁-C₆alkyl;
R³² is

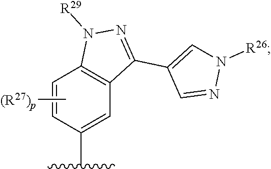

and
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂.

Embodiment 28. The compound of any one of embodiments 25-27, or a pharmaceutically acceptable salt or solvate thereof, wherein:
R³² is

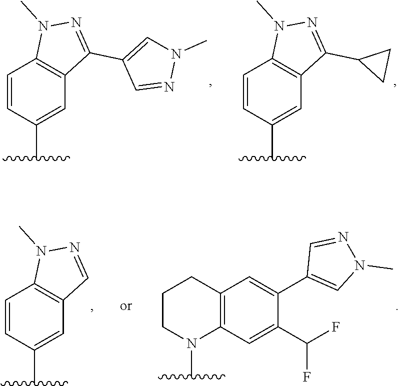 [SMILES: *c1ccc2c(c1)c(-c1cnn(C)c1)nn2C ; *c1ccc2c(c1)c(C1CC1)nn2C ; *c1ccc2c(cnn2C)c1 ; *N1CCCc2cc(-c3cnn(C)c3)c(C(F)F)cc21]

Embodiment 29. The compound of any one of embodiments 12-19, or a pharmaceutically acceptable salt or solvate thereof, wherein the moiety comprising an acetyl-lysine mimetic comprises

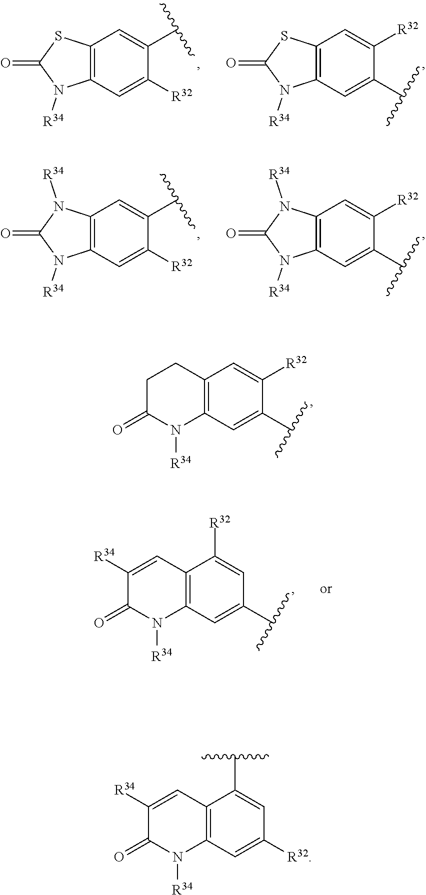

Embodiment 30. The compound of embodiment 29, or a pharmaceutically acceptable salt or solvate thereof, wherein the moiety comprising an acetyl-lysine mimetic comprises

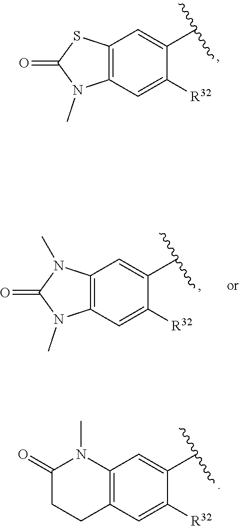

Embodiment 31. The compound of any one of embodiments 29-30, or a pharmaceutically acceptable salt or solvate thereof, wherein:
R³² is

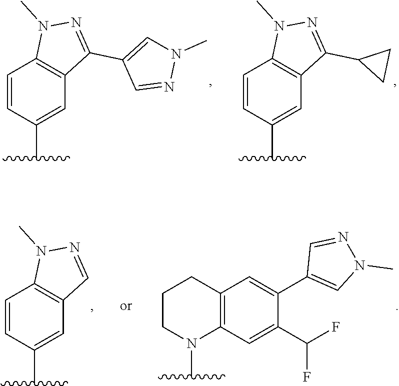 [SMILES: *c1ccc2c(c1)c(-c1cnn(C)c1)nn2C ; *c1ccc2c(c1)c(C1CC1)nn2C ; *c1ccc2c(cnn2C)c1 ; *N1CCCc2cc(-c3cnn(C)c3)c(C(F)F)cc21]

Embodiment 32. The compound of any one of embodiments 12-19, or a pharmaceutically acceptable salt or solvate thereof, wherein the moiety comprising an acetyl-lysine mimetic comprises:

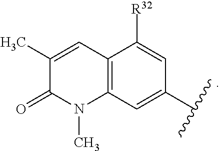

Embodiment 33. The compound of embodiment 32, or a pharmaceutically acceptable salt or solvate thereof, wherein:
R³² is

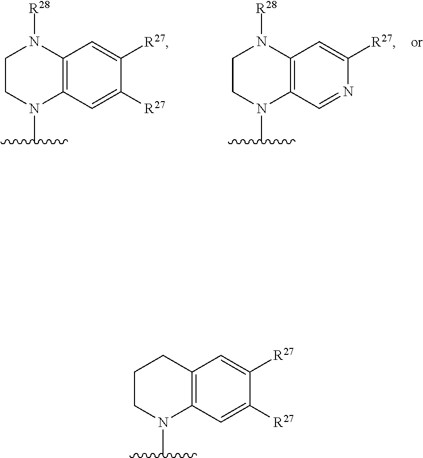

Embodiment 34. The compound of any one of embodiments 29, 30, 32, or 33, or a pharmaceutically acceptable salt or solvate thereof, wherein:
R³² is

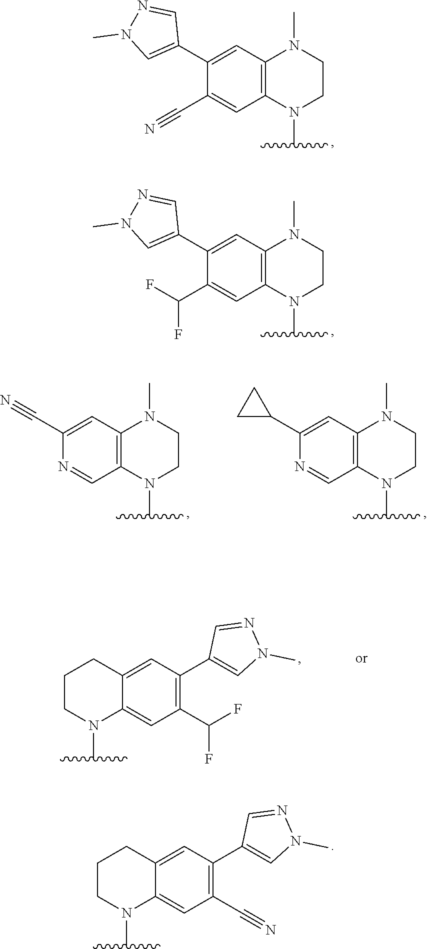 [SMILES: *N1CCN(C)c2cc(-c3cnn(C)c3)c(C#N)cc21 ; *N1CCN(C)c2cc(-c3cnn(C)c3)c(C(F)F)cc21 ; *N1CCN(C)c2cc(C#N)ncc21 ; *N1CCN(C)c2cc(C3CC3)ncc21 ; *N1CCCc2cc(-c3cnn(C)c3)c(C(F)F)cc21 ; *N1CCCc2cc(-c3cnn(C)c3)c(C#N)cc21]

Embodiment 35. The compound of any one of embodiments 12-19, or a pharmaceutically acceptable salt or solvate thereof, wherein the moiety comprising an acetyl-lysine mimetic comprises

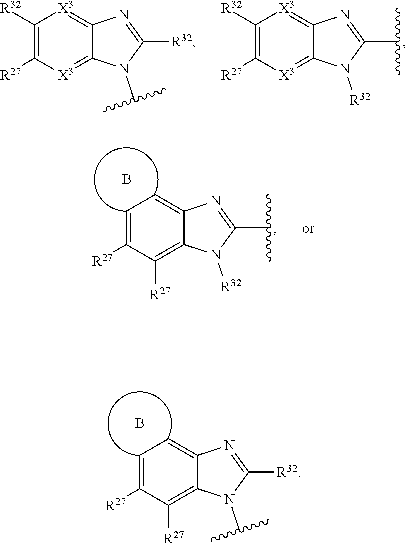

Embodiment 36. The compound of embodiment 35, or a pharmaceutically acceptable salt or solvate thereof, wherein the moiety comprising an acetyl-lysine mimetic comprises

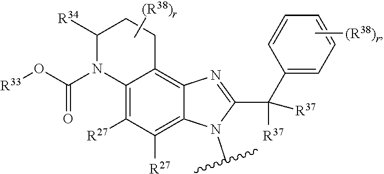

wherein:
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR^{a}, or —N(R^{b})₂;
R³³ is hydrogen, substituted or unsubstituted C₁-C₆alkyl, substituted or unsubstituted C₁-C₆fluoroalkyl, or substituted or unsubstituted C₁-C₆heteroalkyl;
R³⁴ is hydrogen or substituted or unsubstituted C₁-C₆alkyl;
each R³⁸ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, —CN, —NO₂, —OH, —OR^{a}, —OC(═O)R^{a}, —OC(═O)N(R^{b})₂, —OC(═O)OR^{a}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
r is 0, 1, 2, 3, or 4;

Embodiment 37. The compound of embodiment 35, or a pharmaceutically acceptable salt or solvate thereof, wherein:

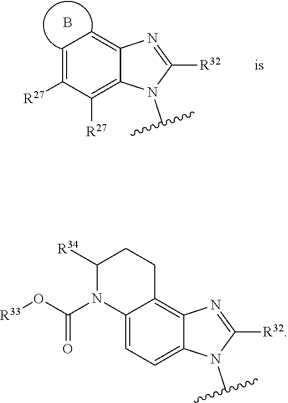

Embodiment 38. The compound of embodiment 35, or a pharmaceutically acceptable salt or solvate thereof, wherein the moiety comprising an acetyl-lysine mimetic comprises

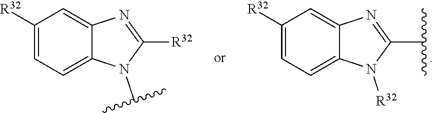

Embodiment 39. The compound of any one of embodiments 35-38, or a pharmaceutically acceptable salt or solvate thereof, wherein:
R³² is

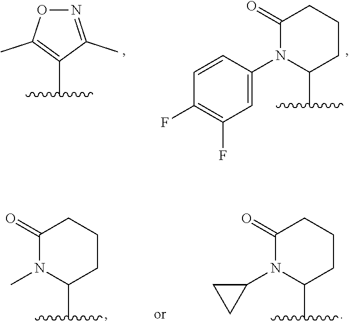

Embodiment 40. The compound of any one of embodiments 35-39, or a pharmaceutically acceptable salt or solvate thereof, wherein:
R³² is

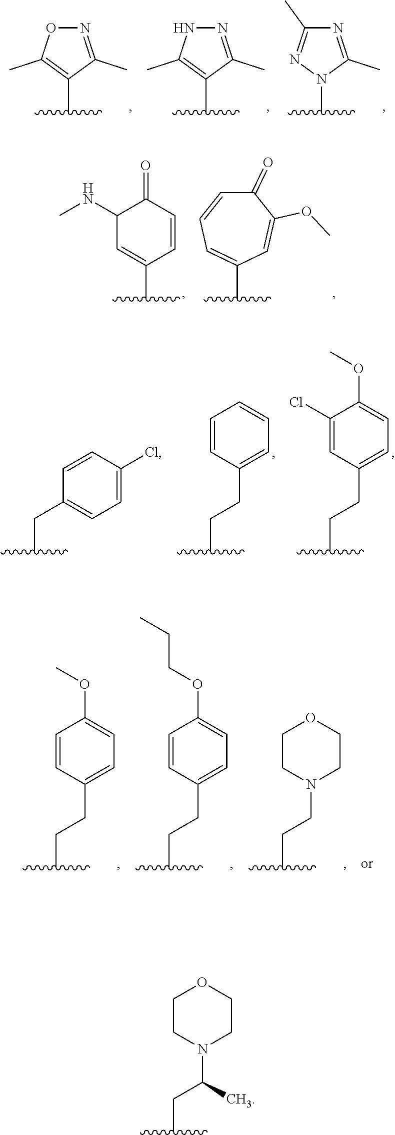

Embodiment 41. The compound of embodiment 12, or a pharmaceutically acceptable salt or solvate thereof, wherein the acetyl-lysine mimetic moiety that binds in the acetyl-lysine (KAc) binding site of the bromodomain of CBP/p300 comprises:
1-(1,4,6,7-tetrahydro-5H-pyrazolo[4,3-c]pyridin-5-yl)ethan-1-one; or N-methyl-1,4,6,7-tetrahydro-5H-pyrazolo[4,3-c]pyridine-5-carboxamide; wherein the acetyl-lysine mimetic moiety optionally further comprises:
   1) a moiety at the 1-position of the 1,4,6,7-tetrahydro-5H-pyrazolo[4,3-c]pyridin-5-yl group that occupies the lipophilic shelf (LPF) region of the bromodomain of CBP/p300; or
   2) a moiety that at the 3-position of the 1,4,6,7-tetrahydro-5H-pyrazolo[4,3-c]pyridin-5-yl group occupies the BC Loop region of the bromodomain of CBP/p300; or
   3) both 1) and 2).

Embodiment 42. The compound of embodiment 12, or a pharmaceutically acceptable salt or solvate thereof, wherein the acetyl-lysine mimetic moiety that binds in the acetyl-lysine (KAc) binding site of the bromodomain of CBP/p300 has the structure:

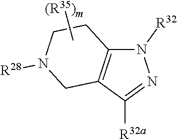

R²⁸ is —C(═O)CH₃, —C(═O)CH₂CH₃, —C(═O)NH₂, —C(═O)NH(CH₃); or —C(═O)NH(CH₂CH₃);
R³² is a moiety that occupies the BC Loop region of the bromodomain of CBP/p300;
R^{32a} is a moiety that occupies the lipophilic shelf (LPF) region of the bromodomain of CBP/p300;
each R³⁵ is independently hydrogen, C₁-C₄alkyl, or C₁-C₄fluoroalkyl;
m is 0, 1, 2, 3, or 4;
L is an optional linker;
wherein L is covalently attached to the R³² group, or at the position occupied by R³², or the R^{32a} group.

Embodiment 43. The compound of embodiment 42, or a pharmaceutically acceptable salt or solvate thereof, wherein:
R³² is hydrogen, substituted or unsubstituted C₁-C₆alkyl, substituted or unsubstituted C₃-C₁₂cycloalkyl, or substituted or unsubstituted 3- to 12-membered heterocycloalkyl;
R^{32a} is

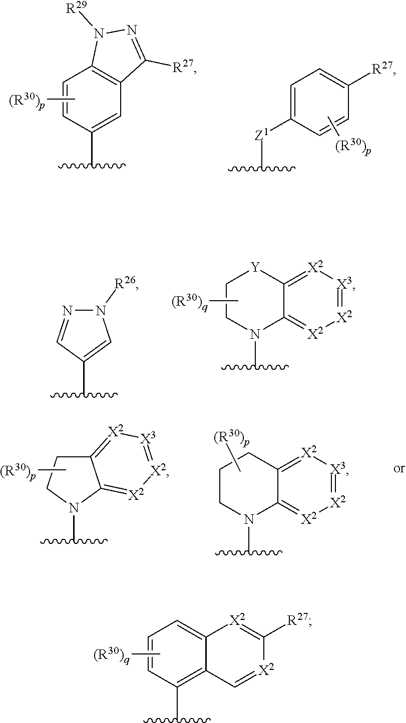

at least one X² is —CR³⁰— and at most two X² are —N—;
Z¹ is —NR^{c}— or —O—;
R^{c} is hydrogen or C₁-C₆alkyl;
R²⁶ is hydrogen, substituted or unsubstituted C₁-C₄alkyl, C₁-C₄fluoroalkyl, substituted or unsubstituted C₃-C₈cycloalkyl, or substituted or unsubstituted 3- to 8-membered heterocycloalkyl;
X³ is —CR²⁷— or —N—;
R²⁷ is hydrogen, halogen, substituted or unsubstituted C₁-C₄alkyl, C₁-C₄fluoroalkyl, substituted or unsubstituted C₃-C₈cycloalkyl, substituted or unsubstituted 3- to 8-membered heterocycloalkyl, substituted or unsubstituted phenyl, substituted or unsubstituted monocyclic heteroaryl, —CN, —OH, —OR^{a}, —N(R^{b})₂, —NR^{b}C(═O)R^{a}, —C(═O)R^{b}, —C(═O)OR^{b}, or —C(═O)N(R^{b})₂;
or R²⁷ is

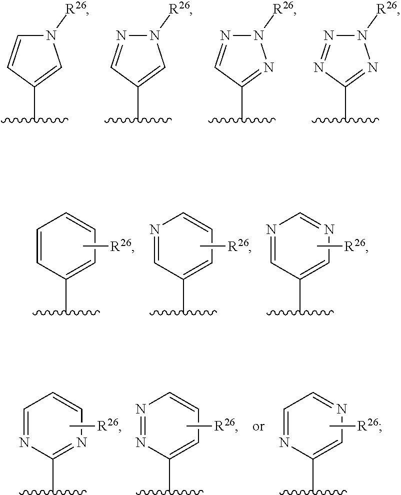

p is 0, 1, 2, or 3;
R²⁹ is hydrogen, substituted or unsubstituted C₁-C₄alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
Y is —C(R³⁰)₂— or N(R²⁸)—;
each R³⁰ is independently hydrogen, halogen, C₁-C₄alkyl, C₁-C₄fluoroalkyl, substituted or unsubstituted C₁-C₆heteroalkyl, —CN, —OH, or —OR^{a};
q is 0, 1, 2, 3, or 4;
each R^{a} is independently C₁-C₄alkyl, C₁-C₆fluoroalkyl, substituted or unsubstituted C₃-C₆cycloalkyl, substituted or unsubstituted monocyclic 3- to 6-membered heterocycloalkyl, substituted or unsubstituted phenyl, or substituted or unsubstituted monocyclic heteroaryl;
each R^{b} is independently hydrogen, C₁-C₄alkyl, C₁-C₄fluoroalkyl, substituted or unsubstituted C₃-C₆cycloalkyl, substituted or unsubstituted monocyclic 3- to 6-membered heterocycloalkyl, substituted or unsubstituted phenyl, or substituted or unsubstituted monocyclic heteroaryl;
or two R^{b} on the same N atom are taken together with the N atom to which they are attached to form a substituted or unsubstituted N-containing heterocycle.

Embodiment 44. The compound of any one of embodiments 42-43, or a pharmaceutically acceptable salt or solvate thereof, wherein:
R²⁷ is hydrogen, halogen, C₁-C₄alkyl, C₁-C₄fluoroalkyl, substituted or unsubstituted C₃-C₈cycloalkyl, substituted or unsubstituted 3- to 8-membered heterocycloalkyl, substituted or unsubstituted phenyl, substituted or unsubstituted monocyclic 5- or 6-membered heteroaryl, —CN, —OH, —OR^{a}, —N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
or R²⁷ is

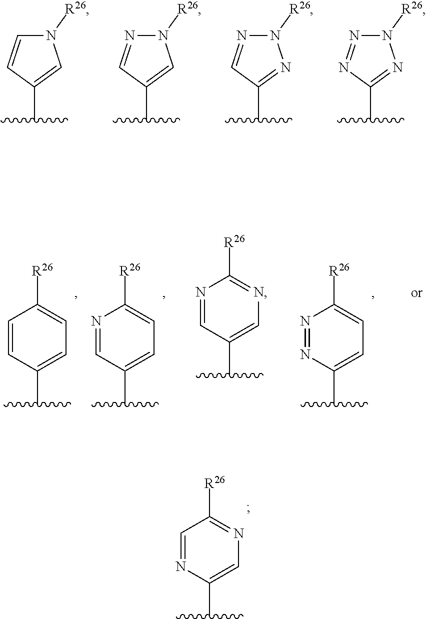

Embodiment 45. The compound of any one of embodiments 42-44, or a pharmaceutically acceptable salt or solvate thereof, wherein:
R³² is

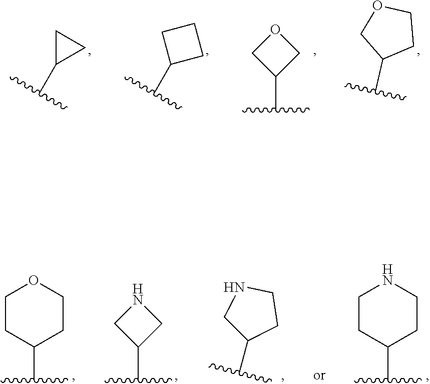

each of which is unsubstituted or substituted with F, Cl, Br, —CH₃, —CD₃, —CH₂CH₃, —CH₂F, —CHF₂, —CF₃, —CH₂CH₂F, —CH₂CHF₂, —CH₂CF₃, —CN, —C(═O)CH₃, —C(═O)CH₂CH₃, —C(═O)CH₂F, —C(═O)CHF₂, —C(═O)CF₃, —C(═O)CH₂CH₂F, —C(═O)CH₂CHF₂, —C(═O)CH₂CF₃, —C(═O)CD₃, —SO₂CH₃, —SO₂CH₂CH₃, —SO₂CD₃, or —SO₂CH₂CD₃;
R^{32a} is

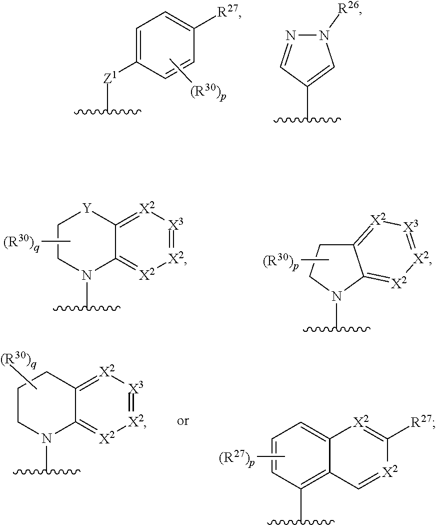

at least one X² is —CR³⁰— and at most two X² are —N—;
each R²⁷ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, substituted or unsubstituted heteroaryl, —CN, —OH, or —OR^{a}; and
each R^{a} is independently substituted or unsubstituted C₁-C₆alkyl.

Embodiment 46. The compound of any one of embodiments 42-45, or a pharmaceutically acceptable salt or solvate thereof, wherein:
R²⁷ is hydrogen, —CH₃, —CH₂CH₃, —CH₂CH₂CH₃, —CH₂(CH₃)₂, —(CH₃)₃, —F, —CHF₂, —CF₃, —CN, —OH, —OCH₃, cyclopropyl, cyclobutyl, oxetanyl, tetrahydrofuranyl, tetrahydropyranyl, unsubstituted or substituted phenyl, unsubstituted or substituted pyrazolyl, unsubstituted or substituted pyridinyl, or unsubstituted or substituted pyrimidinyl; or
R²⁷ is

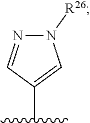

R³² is

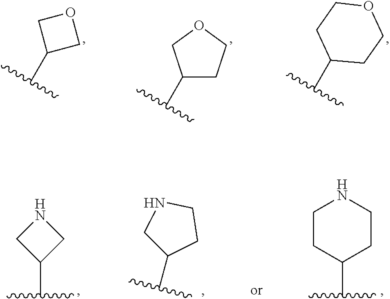 [SMILES: *C1COC1 ; *C1CCOC1 ; *C1CCOCC1 ; *C1CNC1 ; *C1CCNC1 ; *C1CCNCC1]

each of which is unsubstituted or substituted with F, Cl, Br, —CH₃, —CD₃, —CH₂CH₃, —CH₂F, —CHF₂, —CF₃, CN, —C(═O)CH₃, —C(═O)CH₂F, —C(═O)CHF₂, —C(═O)CF₃, or —C(═O)CD₃.

Embodiment 47. The compound of embodiment 42-45, or a pharmaceutically acceptable salt or solvate thereof, wherein:
R³² is

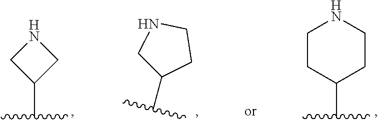 [SMILES: *C1CNC1 ; *C1CCNC1 ; *C1CCNCC1]

wherein the optional linker is covalently attached to the nitrogen of R³² group;
or R³² is absent and L is covalently attached to the acetyl-lysine mimetic moiety at the position occupied by R³²; and
R^{32a} is

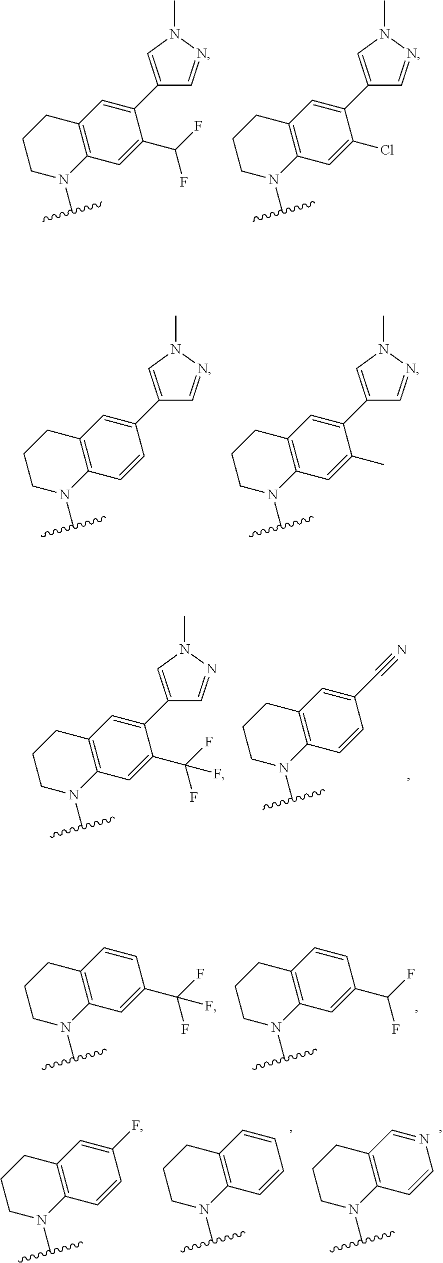

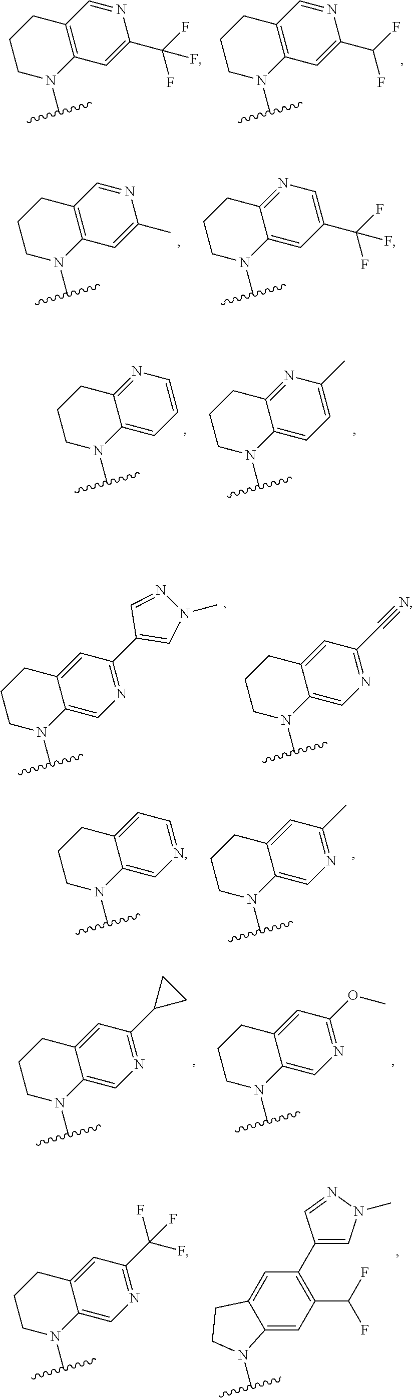

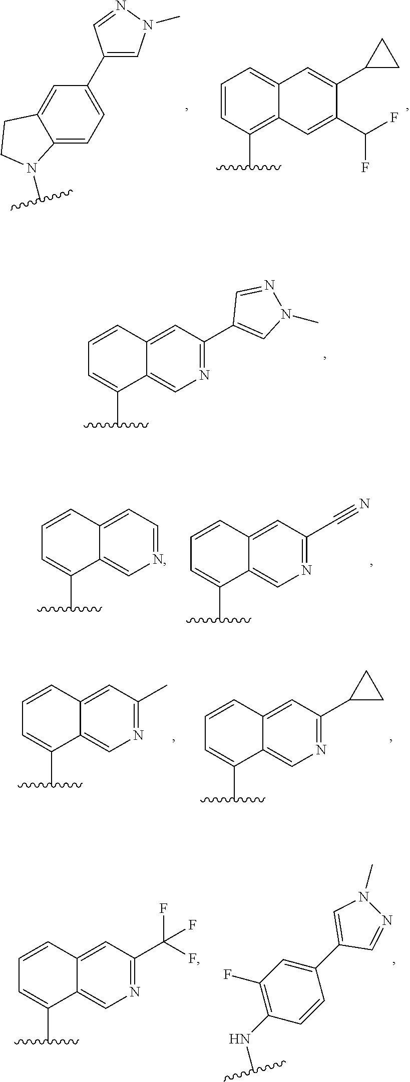

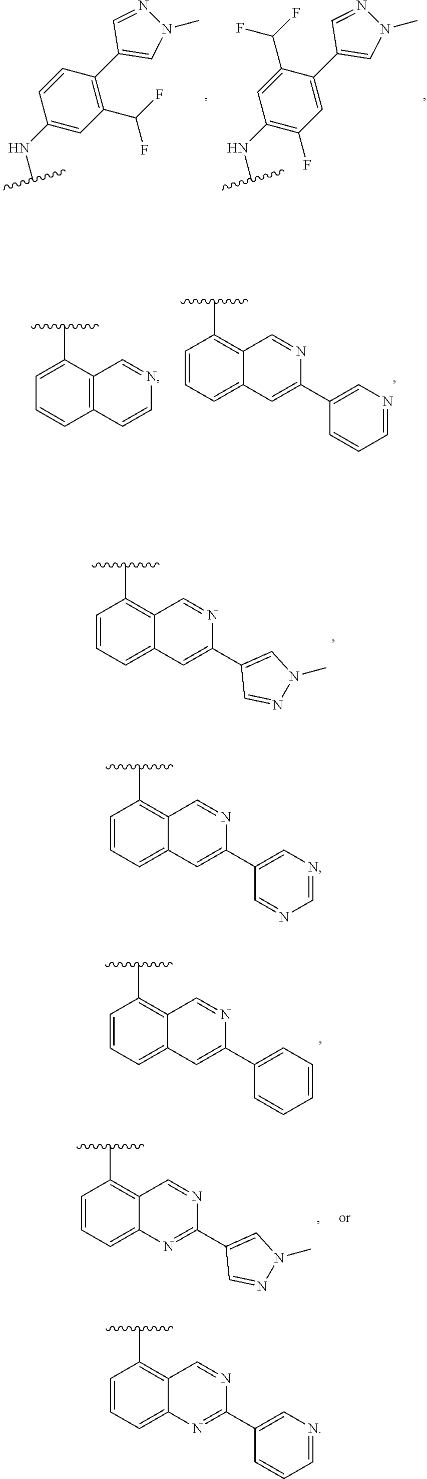

Embodiment 48. The compound of any one of embodiments 42-45, or a pharmaceutically acceptable salt or solvate thereof, wherein:
R³² is

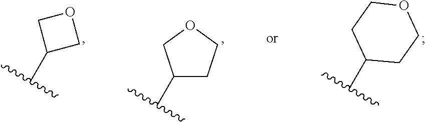 [SMILES: *C1COC1 ; *C1CCOC1 ; *C1CCOCC1]

R^{32a} is

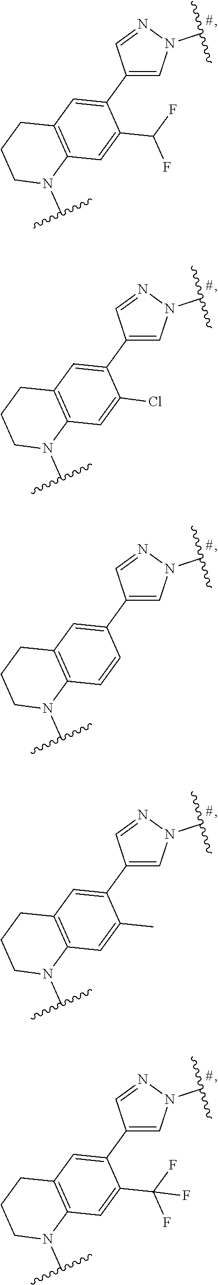 [SMILES: *N1CCCc2cc(-c3cnn(*)c3)c(C(F)F)cc21 ; *N1CCCc2cc(-c3cnn(*)c3)c(Cl)cc21 ; *N1CCCc2cc(-c3cnn(*)c3)ccc21 ; *N1CCCc2cc(-c3cnn(*)c3)c(C)cc21 ; *N1CCCc2cc(-c3cnn(*)c3)c(C(F)(F)F)cc21]

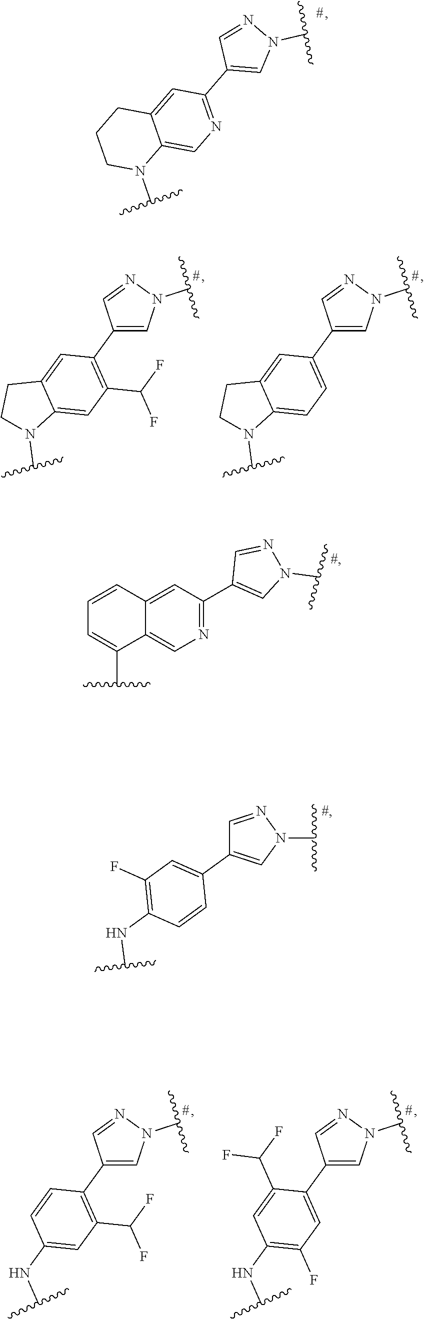

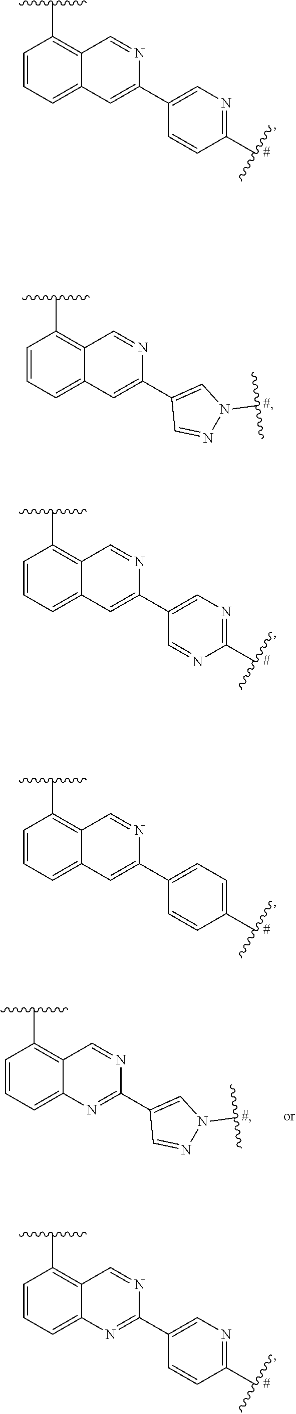 [SMILES: *c1ccc(-c2cc3cccc(*)c3cn2)cn1 ; *c1cccc2cc(-c3cnn(*)c3)ncc12 ; *c1ncc(-c2cc3cccc(*)c3cn2)cn1 ; *c1ccc(-c2cc3cccc(*)c3cn2)cc1 ; *c1cccc2nc(-c3cnn(*)c3)ncc12 ; *c1ccc(-c2ncc3c(*)cccc3n2)cn1]

wherein
[symbol] is the point of attachment to the optional linker.

Embodiment 49. The compound of embodiment 42, or a pharmaceutically acceptable salt or solvate thereof, wherein:
R²⁸ is —C(═O)CH₃ or —C(═O)NH(CH₃);
each R³⁵ is independently hydrogen, —CH₃, —CH₂F, —CHF₂, or —CF₃;
m is 0, 1, or 2;
R³² is

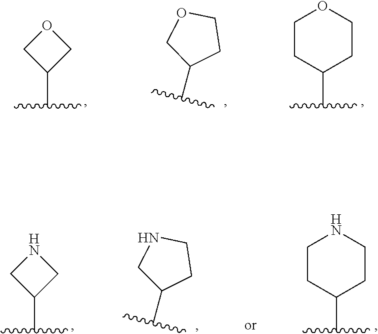 [SMILES: *C1COC1 ; *C1CCOC1 ; *C1CCOCC1 ; *C1CNC1 ; *C1CCNC1 ; *C1CCNCC1]

or each of which is unsubstituted or substituted with F, —CH₃, —CH₂F, —CHF₂, or —CF₃;
R^{32a} is

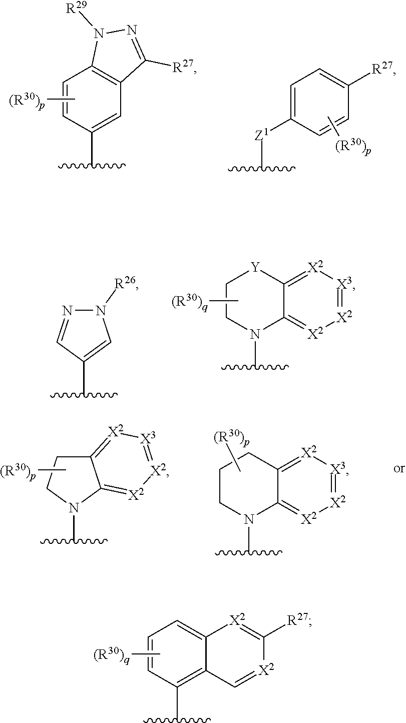

at least one X² is —CR³⁰— and at most two X² are —N—;
R²⁶ is hydrogen, C₁-C₄alkyl, C₁-C₄fluoroalkyl, substituted or unsubstituted C₃-C₆cycloalkyl, or substituted or unsubstituted 3- to 6-membered heterocycloalkyl;
R²⁷ is hydrogen, halogen, C₁-C₄alkyl, C₁-C₄fluoroalkyl, substituted or unsubstituted C₃-C₆cycloalkyl, substituted or unsubstituted 3- to 6-membered heterocycloalkyl, substituted or unsubstituted phenyl, substituted or unsubstituted monocyclic 5- or 6-membered heteroaryl, —CN, —OH, —OR^{a}, or —N(R^{b})₂;
or R²⁷ is

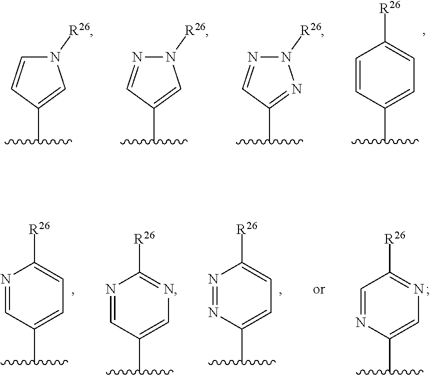

each R³⁰ is independently hydrogen, halogen, C₁-C₄alkyl, C₁-C₄fluoroalkyl, —CN, —OH, or —OR^{a};
R^{a} is C₁-C₄alkyl;
each R^{b} is independently hydrogen or C₁-C₆alkyl;
wherein L is covalently attached to the R³² group, or at the position occupied by R²⁶, or at the position occupied by R²⁷.

Embodiment 50. The compound of embodiment 49, or a pharmaceutically acceptable salt or solvate thereof, wherein:
R³² is

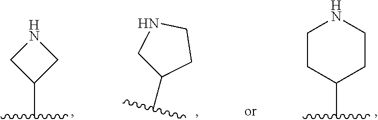 [SMILES: *C1CNC1 ; *C1CCNC1 ; *C1CCNCC1]

wherein the optional linker is covalently attached to the nitrogen of R³² group;
or R³² is absent and the optional linker is covalently attached to the acetyl-lysine mimetic moiety at the position occupied by R³²;
R^{32a} is

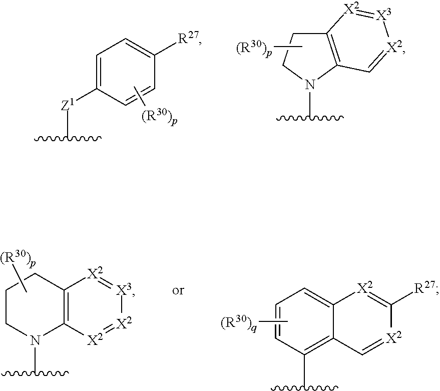

at least one X² is —CR³⁰— and at most two X² are —N—;
X³ is —CR²⁷— or —N—;
Z¹ is —NH— or —O—;
R²⁶ is hydrogen, C₁-C₄alkyl, C₁-C₄fluoroalkyl, substituted or unsubstituted C₃-C₆cycloalkyl, or substituted or unsubstituted 3- to 6-membered heterocycloalkyl;
R²⁷ is hydrogen, halogen, C₁-C₄alkyl, C₁-C₄fluoroalkyl, substituted or unsubstituted C₃-C₆cycloalkyl, substituted or unsubstituted 3- to 6-membered heterocycloalkyl, substituted or unsubstituted phenyl, substituted or unsubstituted monocyclic 5- or 6-membered heteroaryl, —CN, —OH, —OR^{a}, or —N(R^{b})₂;
or R²⁷ is

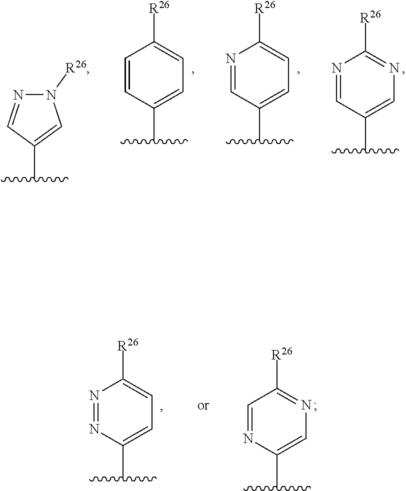

and
each R³⁰ is independently hydrogen, halogen, C₁-C₄alkyl, C₁-C₄fluoroalkyl, —CN, —OH, or —OR^{a}.

Embodiment 51. The compound of embodiment 49, or a pharmaceutically acceptable salt or solvate thereof, wherein:
R³² is

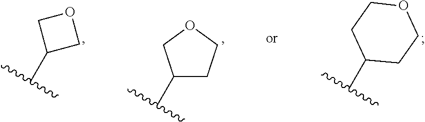 [SMILES: *C1COC1 ; *C1CCOC1 ; *C1CCOCC1]

R^{32a} is

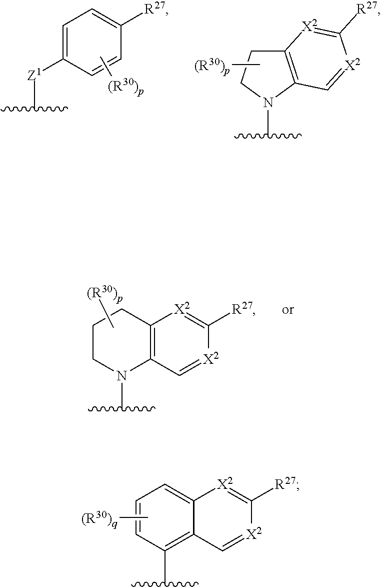

R²⁷ is

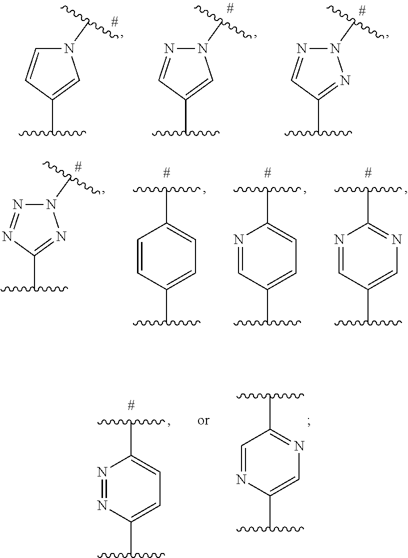

Z¹ is —NH— or —O—; and
[symbol] is the point of attachment to the optional linker.

Embodiment 52. The compound of any one of embodiments 9-14, or a pharmaceutically acceptable salt or solvate thereof, wherein SB-CBP/p300 has the structure of Formula (IIIb):

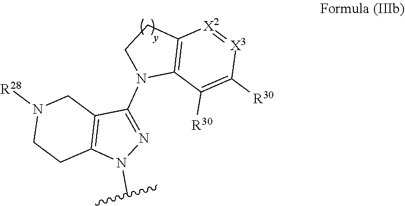

wherein:
each X² is independently —CR³⁰— or —N—;
X³ is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
each R³⁰ is independently hydrogen, halogen, C₁-C₄alkyl, C₁-C₄fluoroalkyl, —CN, —OH, or —OR^{a};
y is 1 or 2;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 53. The compound of any one of embodiments 9-14, or a pharmaceutically acceptable salt or solvate thereof, wherein SB-CBP/p300 has the structure of Formula (IIIc):

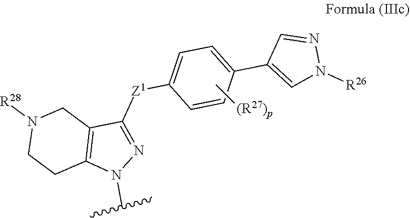

wherein:
R²⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{a}, or —C(═O)N(R^{b})₂;
Z¹ is —NR^{c}—, —O—, or —S—;
R^{c} is hydrogen or substituted or unsubstituted C₁-C₆alkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl; and
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 54. The compound of any one of embodiments 9-14, or a pharmaceutically acceptable salt or solvate thereof, wherein SB-CBP/p300 has the structure of Formula (IIId-1) or (IIId-2):

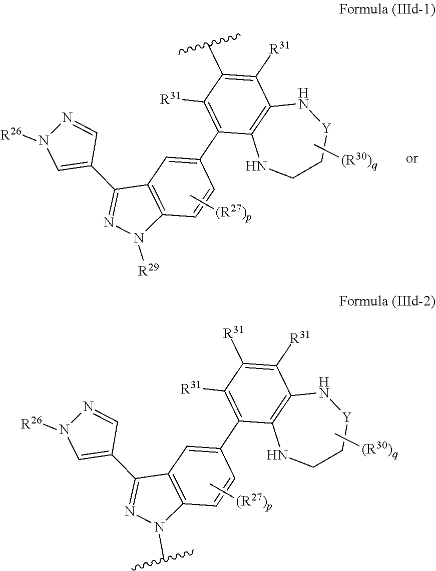

wherein:
R²⁶ is hydrogen, substituted or unsubstituted C₁-C₆alkyl, substituted or unsubstituted C₁-C₆heteroalkyl, substituted or unsubstituted C₃-C₈cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
each R³¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)NR^{b}, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
Y is —C(R³⁰)₂— or C(═O);
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 55. The compound of any one of embodiments 9-14, or a pharmaceutically acceptable salt or solvate thereof, wherein SB-CBP/p300 has the structure of Formula (IIIe):

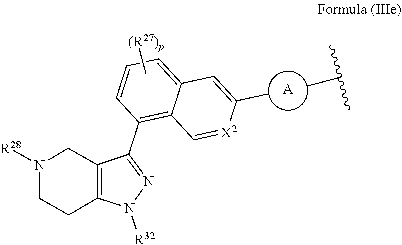

wherein:
X² is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
R³² is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
Ring A is absent or substituted or unsubstituted C₃-C₈cycloalkyl, substituted or unsubstituted 3- to 8-membered heterocycloalkyl, substituted or unsubstituted aryl, or substituted or unsubstituted heteroaryl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 56. The compound of any one of embodiments 9-14, or a pharmaceutically acceptable salt or solvate thereof, wherein SB-CBP/p300 has one of the following structures:

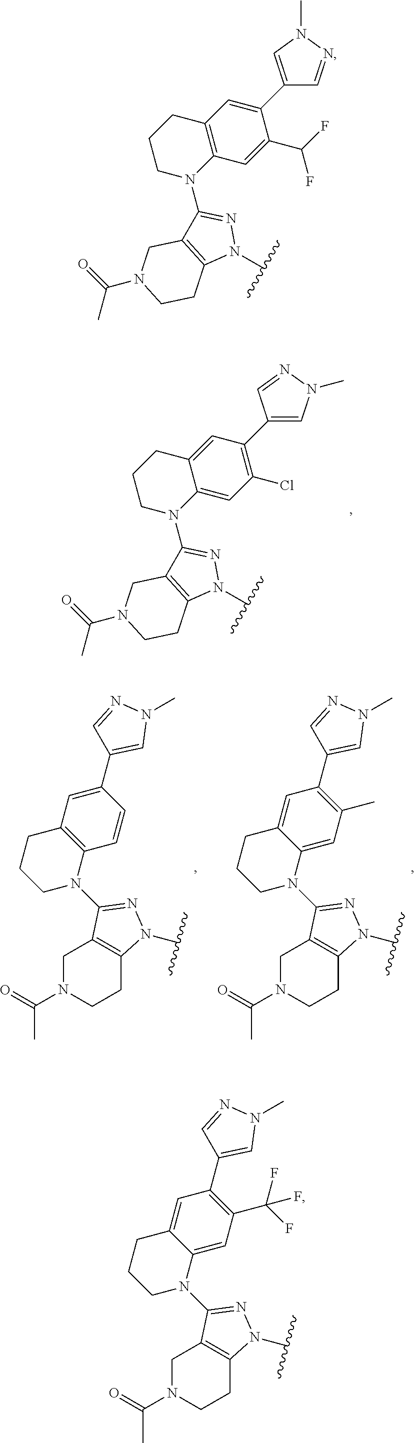 [SMILES: *n1nc(N2CCCc3cc(-c4cnn(C)c4)c(C(F)F)cc32)c2c1CCN(C(C)=O)C2 ; *n1nc(N2CCCc3cc(-c4cnn(C)c4)c(Cl)cc32)c2c1CCN(C(C)=O)C2 ; *n1nc(N2CCCc3cc(-c4cnn(C)c4)ccc32)c2c1CCN(C(C)=O)C2 ; *n1nc(N2CCCc3cc(-c4cnn(C)c4)c(C)cc32)c2c1CCN(C(C)=O)C2 ; *n1nc(N2CCCc3cc(-c4cnn(C)c4)c(C(F)(F)F)cc32)c2c1CCN(C(C)=O)C2]

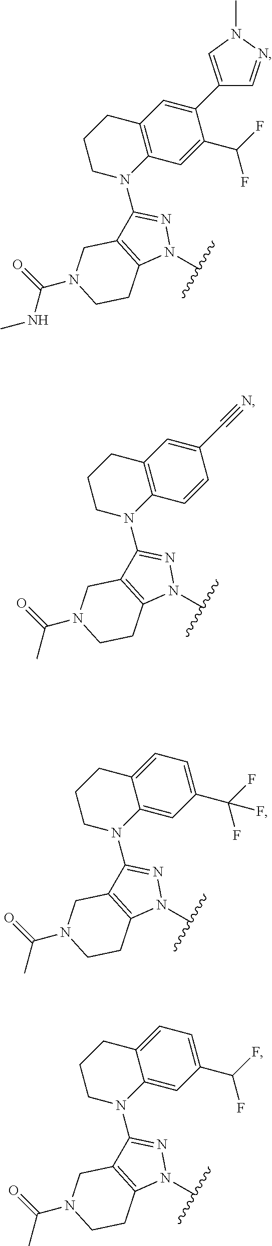 [SMILES: *n1nc(N2CCCc3cc(-c4cnn(C)c4)c(C(F)F)cc32)c2c1CCN(C(=O)NC)C2 ; *n1nc(N2CCCc3cc(C#N)ccc32)c2c1CCN(C(C)=O)C2 ; *n1nc(N2CCCc3ccc(C(F)(F)F)cc32)c2c1CCN(C(C)=O)C2 ; *n1nc(N2CCCc3ccc(C(F)F)cc32)c2c1CCN(C(C)=O)C2]

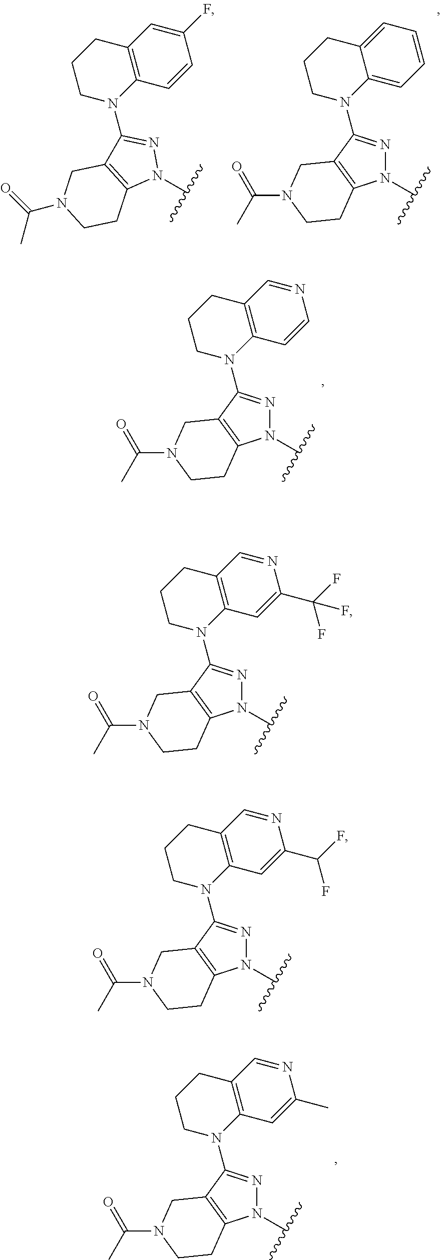 [SMILES: *n1nc(N2CCCc3cc(F)ccc32)c2c1CCN(C(C)=O)C2 ; *n1nc(N2CCCc3ccccc32)c2c1CCN(C(C)=O)C2 ; *n1nc(N2CCCc3cnccc32)c2c1CCN(C(C)=O)C2 ; *n1nc(N2CCCc3cnc(C(F)(F)F)cc32)c2c1CCN(C(C)=O)C2 ; *n1nc(N2CCCc3cnc(C(F)F)cc32)c2c1CCN(C(C)=O)C2 ; *n1nc(N2CCCc3cnc(C)cc32)c2c1CCN(C(C)=O)C2]

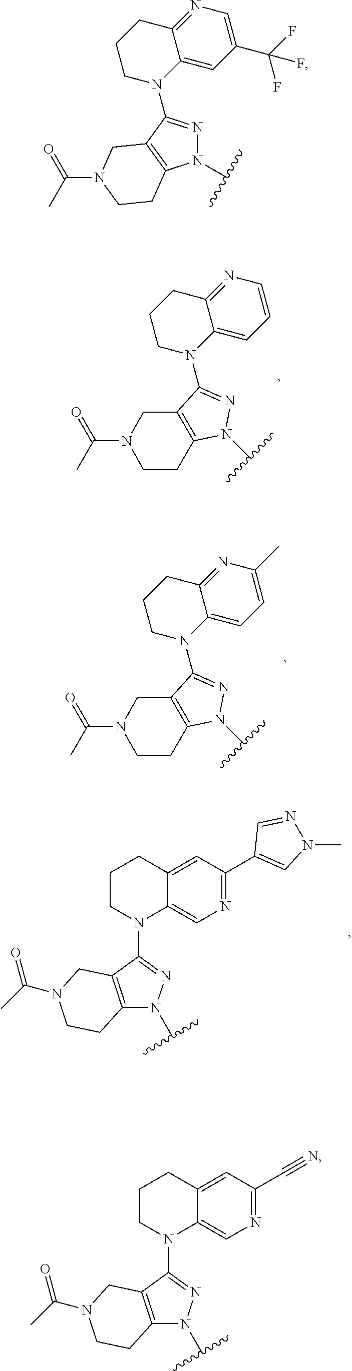

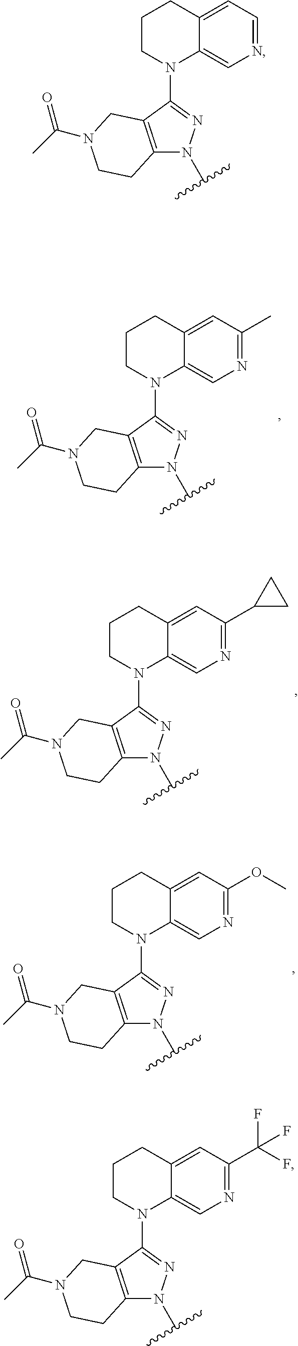

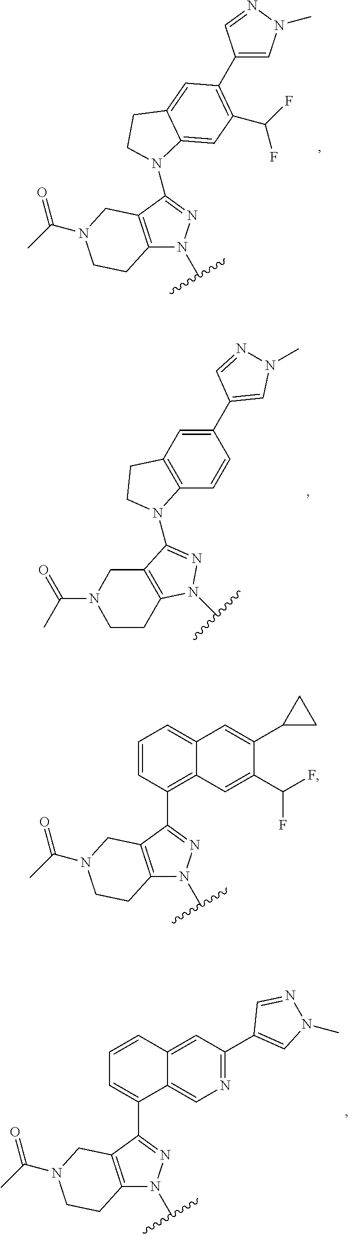

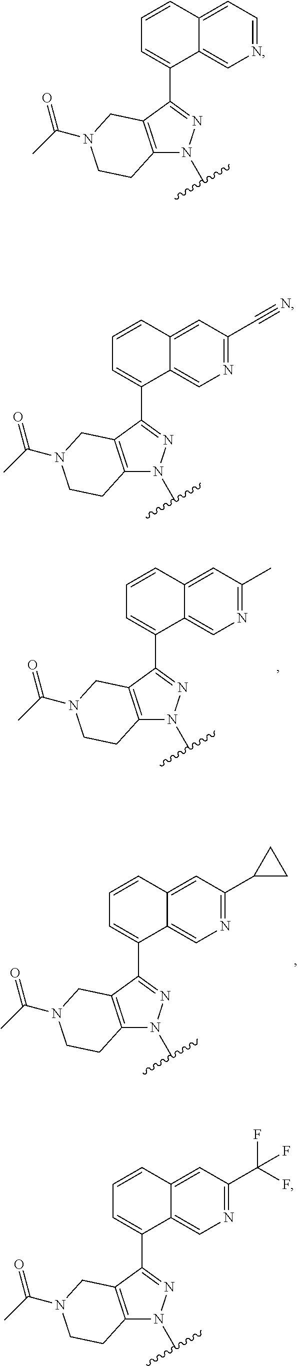

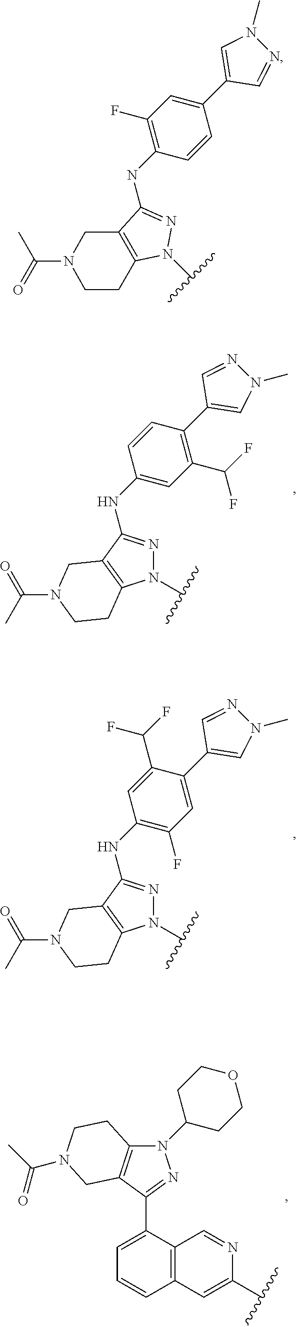

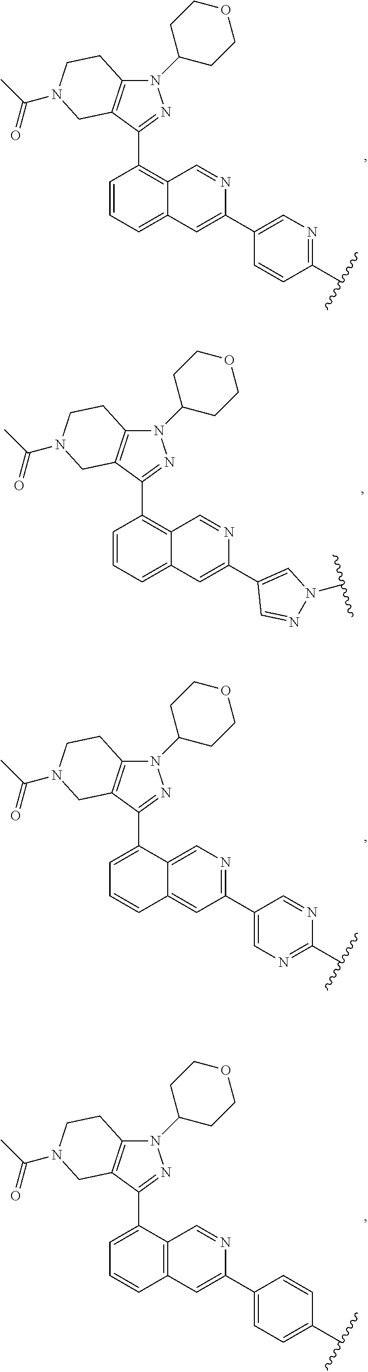 [SMILES: *c1ccc(-c2cc3cccc(-c4nn(C5CCOCC5)c5c4CN(C(C)=O)CC5)c3cn2)cn1 ; *n1cc(-c2cc3cccc(-c4nn(C5CCOCC5)c5c4CN(C(C)=O)CC5)c3cn2)cn1 ; *c1ncc(-c2cc3cccc(-c4nn(C5CCOCC5)c5c4CN(C(C)=O)CC5)c3cn2)cn1 ; *c1ccc(-c2cc3cccc(-c4nn(C5CCOCC5)c5c4CN(C(C)=O)CC5)c3cn2)cc1]

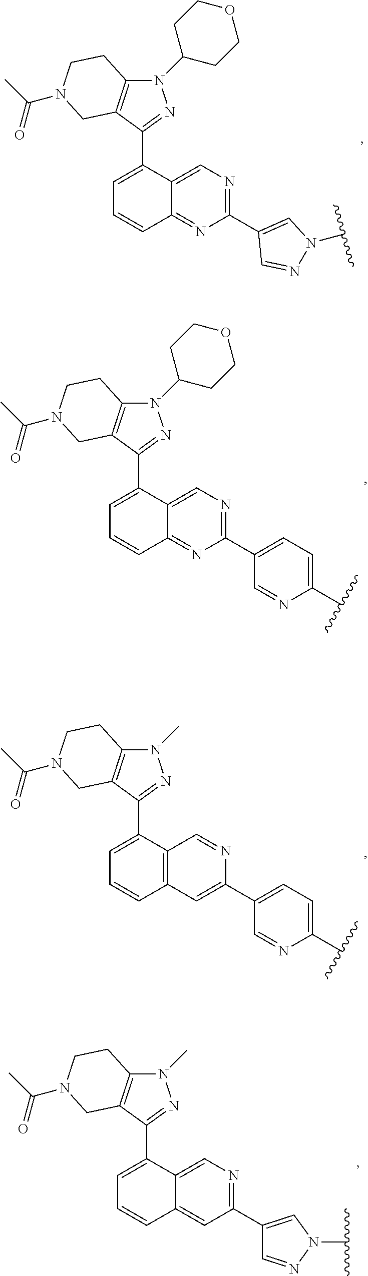 [SMILES: *n1cc(-c2ncc3c(-c4nn(C5CCOCC5)c5c4CN(C(C)=O)CC5)cccc3n2)cn1 ; *c1ccc(-c2ncc3c(-c4nn(C5CCOCC5)c5c4CN(C(C)=O)CC5)cccc3n2)cn1 ; *c1ccc(-c2cc3cccc(-c4nn(C)c5c4CN(C(C)=O)CC5)c3cn2)cn1 ; *n1cc(-c2cc3cccc(-c4nn(C)c5c4CN(C(C)=O)CC5)c3cn2)cn1]

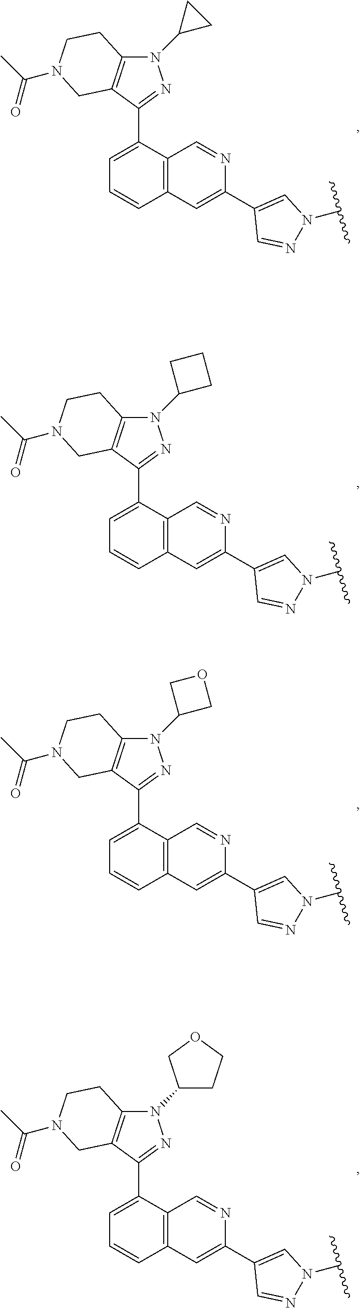 [SMILES: *n1cc(-c2cc3cccc(-c4nn(C5CC5)c5c4CN(C(C)=O)CC5)c3cn2)cn1 ; *n1cc(-c2cc3cccc(-c4nn(C5CCC5)c5c4CN(C(C)=O)CC5)c3cn2)cn1 ; *n1cc(-c2cc3cccc(-c4nn(C5COC5)c5c4CN(C(C)=O)CC5)c3cn2)cn1 ; *n1cc(-c2cc3cccc(-c4nn([C@H]5CCOC5)c5c4CN(C(C)=O)CC5)c3cn2)cn1]

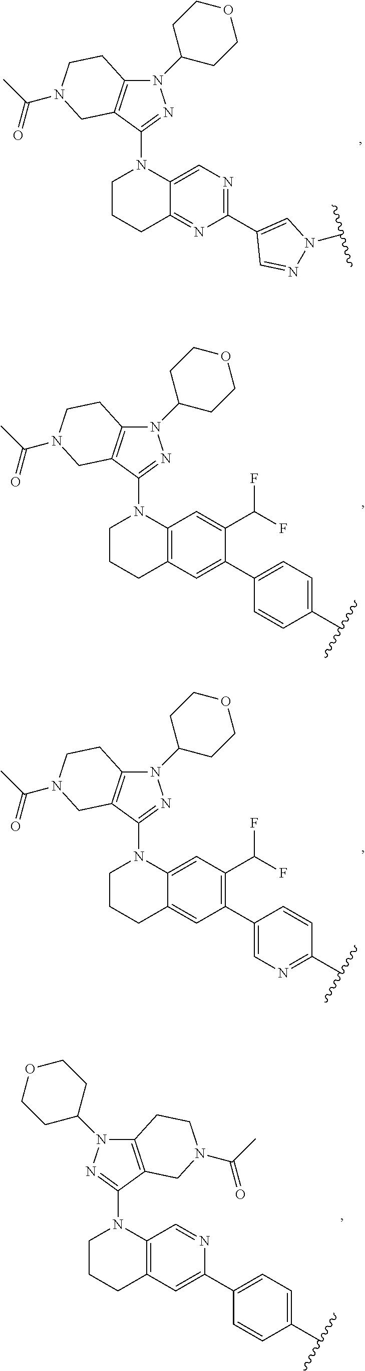 [SMILES: *n1cc(-c2ncc3c(n2)CCCN3c2nn(C3CCOCC3)c3c2CN(C(C)=O)CC3)cn1 ; *c1ccc(-c2cc3c(cc2C(F)F)N(c2nn(C4CCOCC4)c4c2CN(C(C)=O)CC4)CCC3)cc1 ; *c1ccc(-c2cc3c(cc2C(F)F)N(c2nn(C4CCOCC4)c4c2CN(C(C)=O)CC4)CCC3)cn1 ; *c1ccc(-c2cc3c(cn2)N(c2nn(C4CCOCC4)c4c2CN(C(C)=O)CC4)CCC3)cc1]

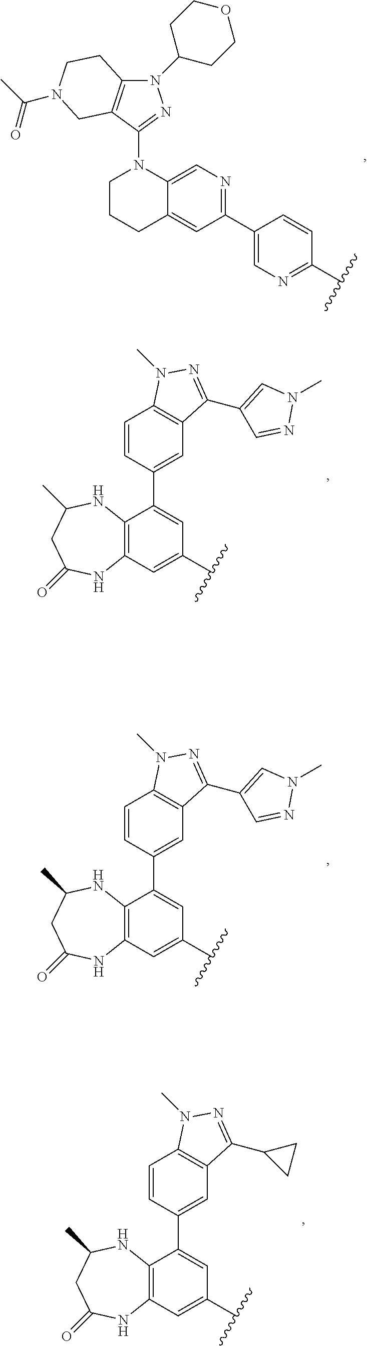 [SMILES: *c1ccc(-c2cc3c(cn2)N(c2nn(C4CCOCC4)c4c2CN(C(C)=O)CC4)CCC3)cn1 ; *c1cc2c(c(-c3ccc4c(c3)c(-c3cnn(C)c3)nn4C)c1)NC(C)CC(=O)N2 ; *c1cc2c(c(-c3ccc4c(c3)c(-c3cnn(C)c3)nn4C)c1)N[C@H](C)CC(=O)N2 ; *c1cc2c(c(-c3ccc4c(c3)c(C3CC3)nn4C)c1)N[C@H](C)CC(=O)N2]

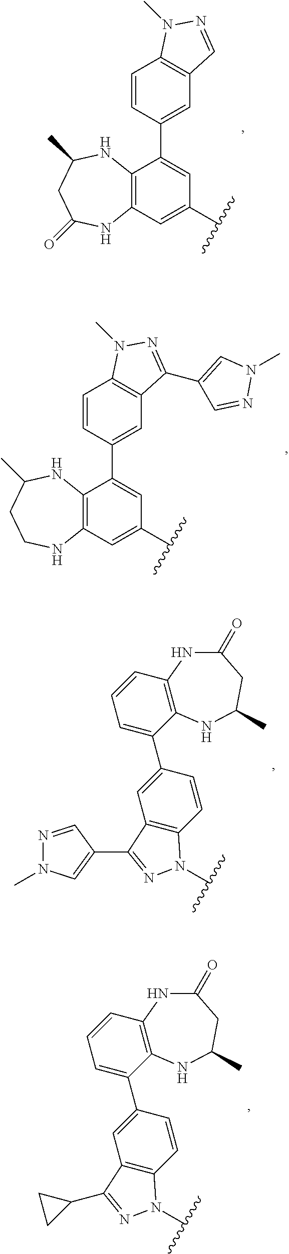

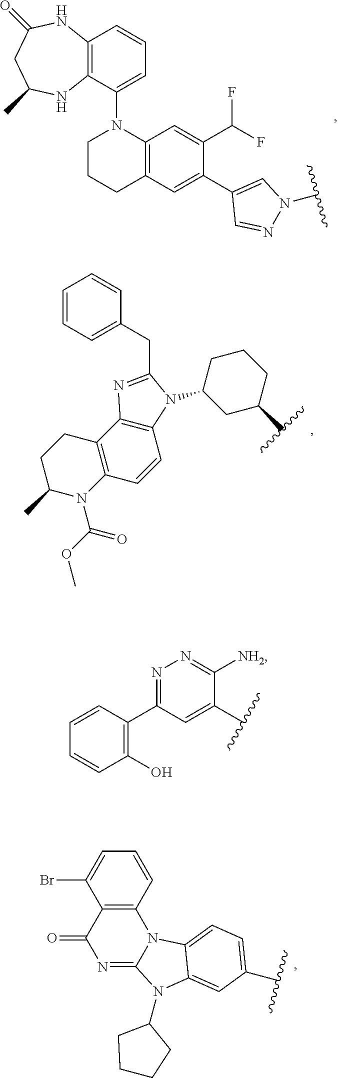 [SMILES: *n1cc(-c2cc3c(cc2C(F)F)N(c2cccc4c2N[C@@H](C)CC(=O)N4)CCC3)cn1 ; *[C@@H]1CCC[C@@H](n2c(Cc3ccccc3)nc3c4c(ccc32)N(C(=O)OC)[C@@H](C)CC4)C1 ; *c1cc(-c2ccccc2O)nnc1N ; *c1ccc2c(c1)n(C1CCCC1)c1nc(=O)c3c(Br)cccc3n21]

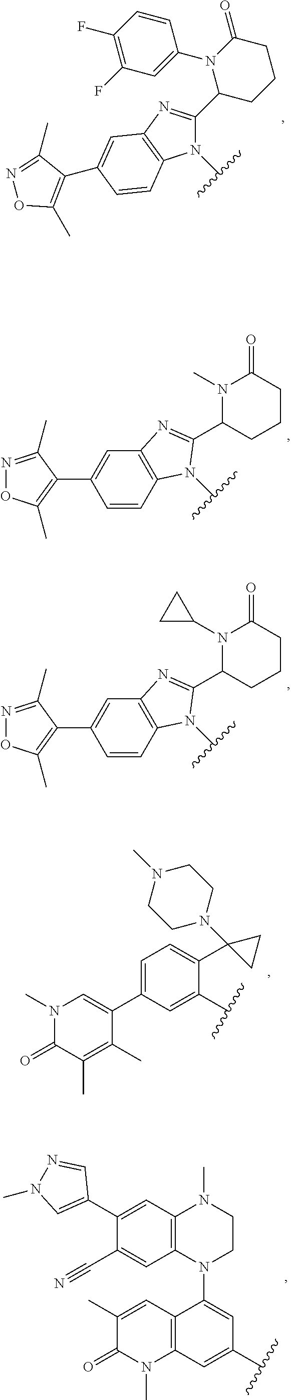 [SMILES: *n1c(C2CCCC(=O)N2c2ccc(F)c(F)c2)nc2cc(-c3c(C)noc3C)ccc21 ; *n1c(C2CCCC(=O)N2C)nc2cc(-c3c(C)noc3C)ccc21 ; *n1c(C2CCCC(=O)N2C2CC2)nc2cc(-c3c(C)noc3C)ccc21 ; *c1cc(-c2cn(C)c(=O)c(C)c2C)ccc1C1(N2CCN(C)CC2)CC1 ; *c1cc(N2CCN(C)c3cc(-c4cnn(C)c4)c(C#N)cc32)c2cc(C)c(=O)n(C)c2c1]

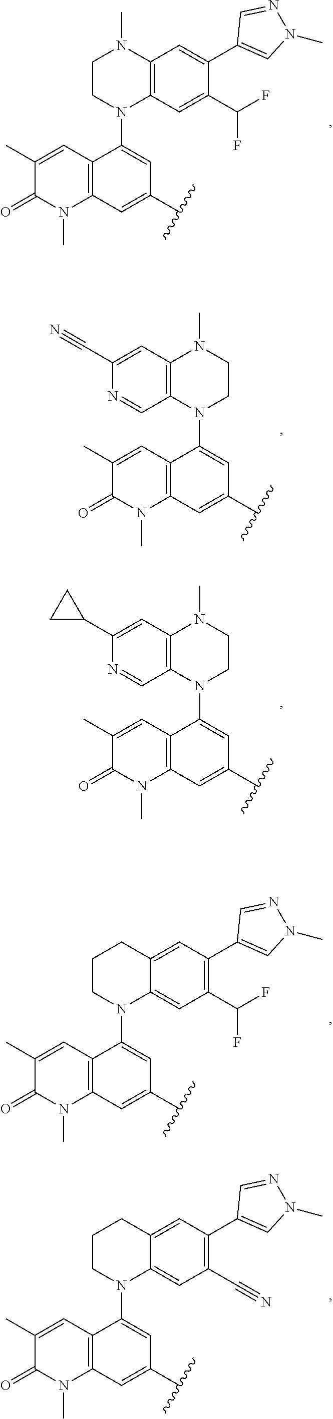 [SMILES: *c1cc(N2CCN(C)c3cc(-c4cnn(C)c4)c(C(F)F)cc32)c2cc(C)c(=O)n(C)c2c1 ; *c1cc(N2CCN(C)c3cc(C#N)ncc32)c2cc(C)c(=O)n(C)c2c1 ; *c1cc(N2CCN(C)c3cc(C4CC4)ncc32)c2cc(C)c(=O)n(C)c2c1 ; *c1cc(N2CCCc3cc(-c4cnn(C)c4)c(C(F)F)cc32)c2cc(C)c(=O)n(C)c2c1 ; *c1cc(N2CCCc3cc(-c4cnn(C)c4)c(C#N)cc32)c2cc(C)c(=O)n(C)c2c1]

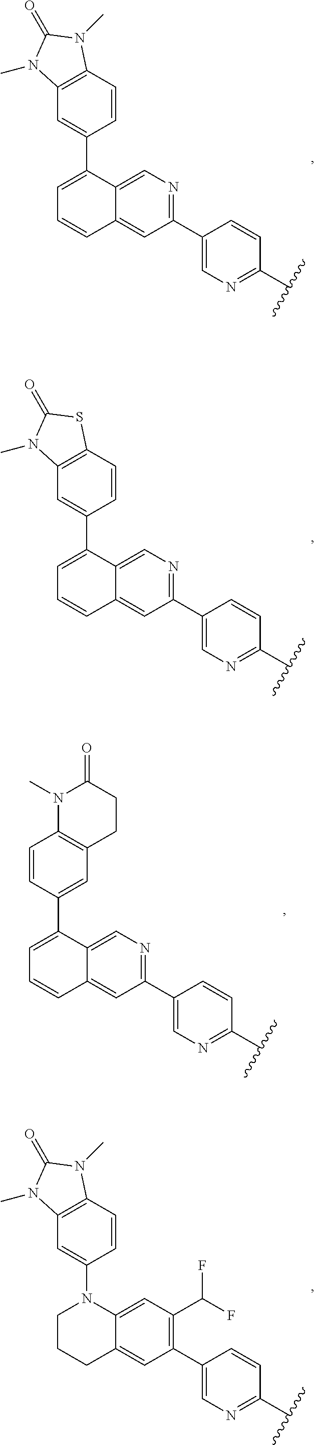 [SMILES: *c1ccc(-c2cc3cccc(-c4ccc5c(c4)n(C)c(=O)n5C)c3cn2)cn1 ; *c1ccc(-c2cc3cccc(-c4ccc5sc(=O)n(C)c5c4)c3cn2)cn1 ; *c1ccc(-c2cc3cccc(-c4ccc5c(c4)CCC(=O)N5C)c3cn2)cn1 ; *c1ccc(-c2cc3c(cc2C(F)F)N(c2ccc4c(c2)n(C)c(=O)n4C)CCC3)cn1]

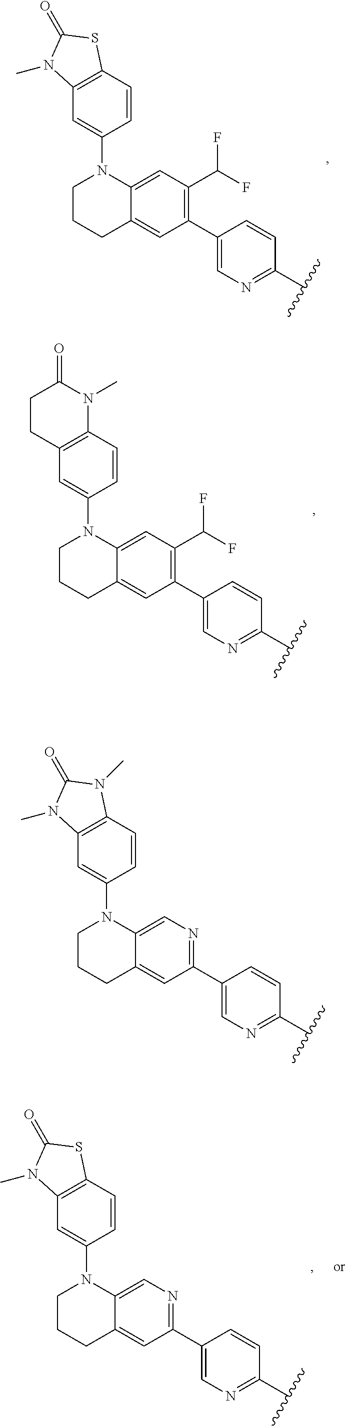 [SMILES: *c1ccc(-c2cc3c(cc2C(F)F)N(c2ccc4sc(=O)n(C)c4c2)CCC3)cn1 ; *c1ccc(-c2cc3c(cc2C(F)F)N(c2ccc4c(c2)CCC(=O)N4C)CCC3)cn1 ; *c1ccc(-c2cc3c(cn2)N(c2ccc4c(c2)n(C)c(=O)n4C)CCC3)cn1 ; *c1ccc(-c2cc3c(cn2)N(c2ccc4sc(=O)n(C)c4c2)CCC3)cn1]

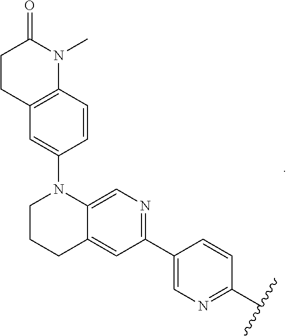 [SMILES: *c1ccc(-c2cc3c(cn2)N(c2ccc4c(c2)CCC(=O)N4C)CCC3)cn1]

Embodiment 57. The compound of any one of embodiments 9-14, wherein SB-CBP/p300 has one of the following structures:

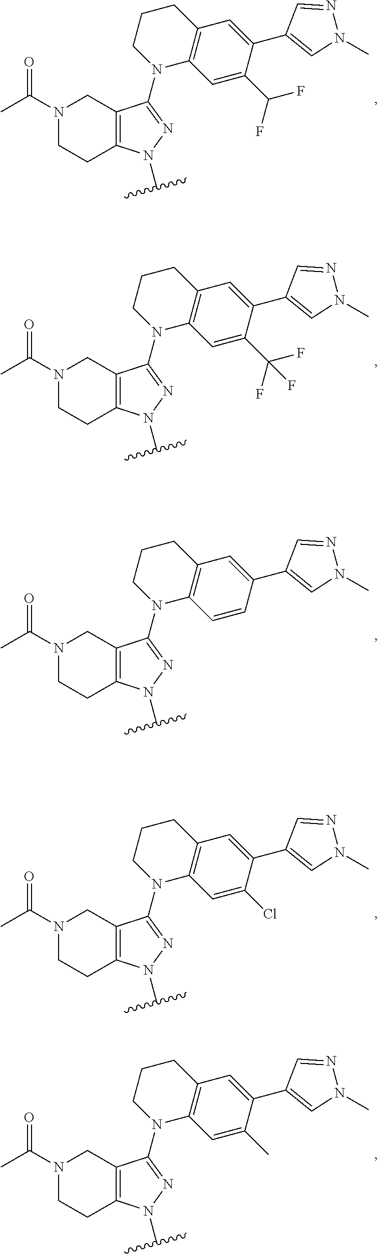

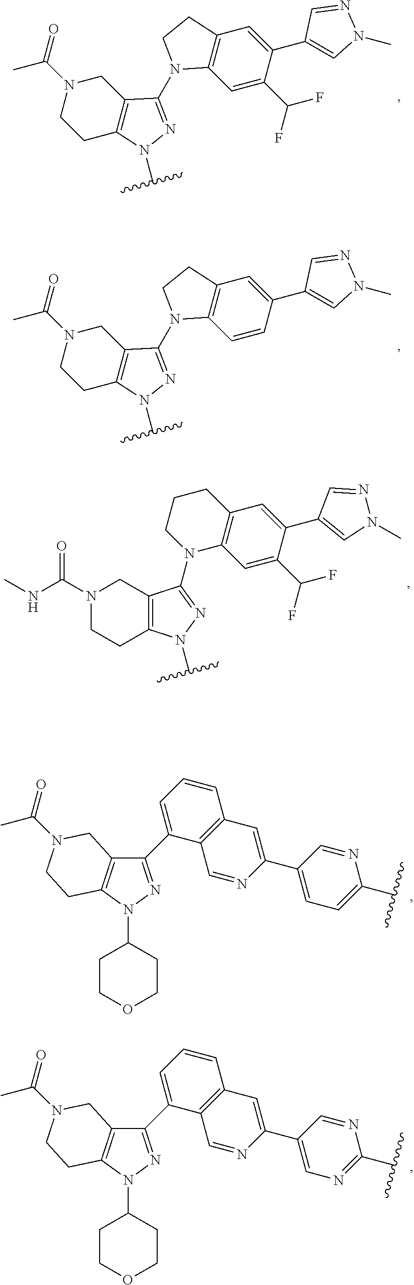

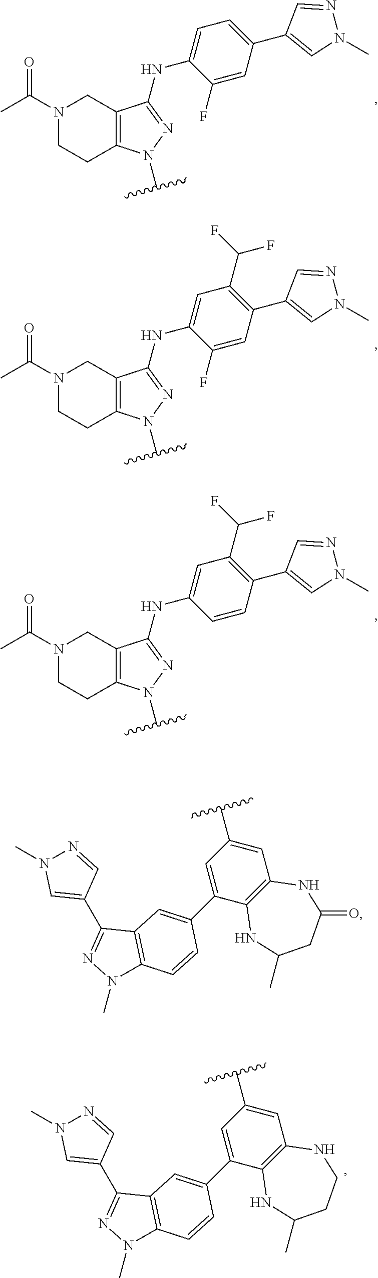

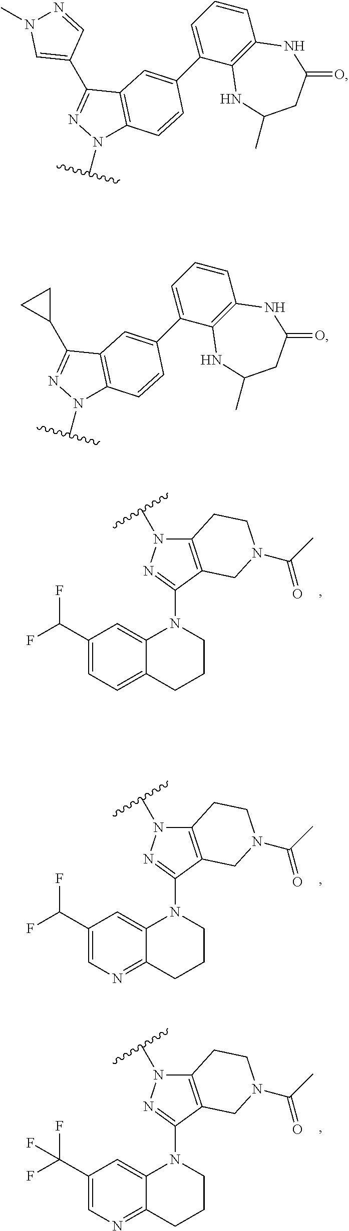 [SMILES: *n1nc(-c2cnn(C)c2)c2cc(-c3cccc4c3NC(C)CC(=O)N4)ccc21 ; *n1nc(C2CC2)c2cc(-c3cccc4c3NC(C)CC(=O)N4)ccc21 ; *n1nc(N2CCCc3ccc(C(F)F)cc32)c2c1CCN(C(C)=O)C2 ; *n1nc(N2CCCc3ncc(C(F)F)cc32)c2c1CCN(C(C)=O)C2 ; *n1nc(N2CCCc3ncc(C(F)(F)F)cc32)c2c1CCN(C(C)=O)C2]

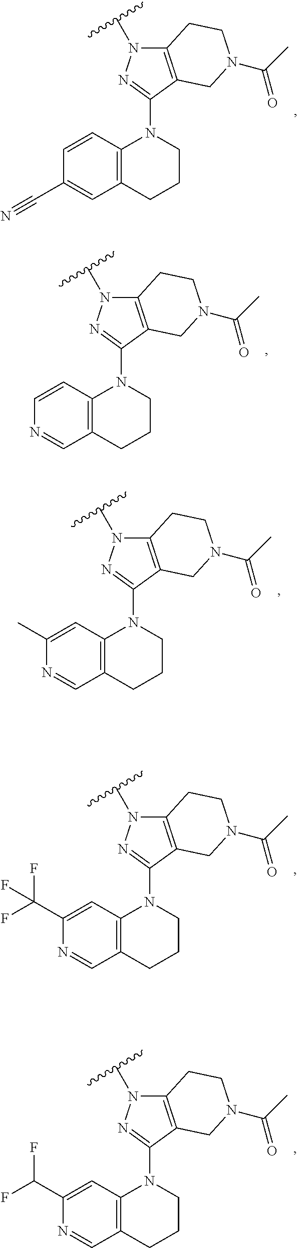

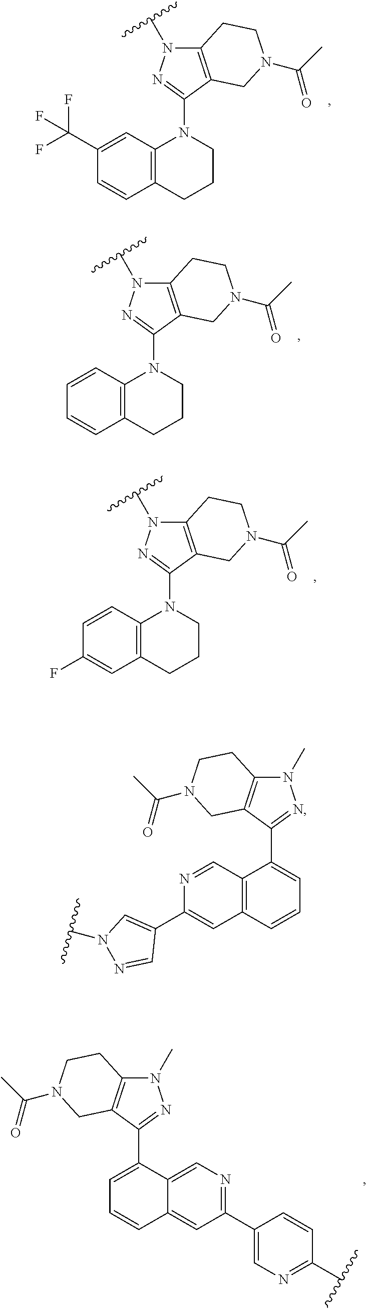

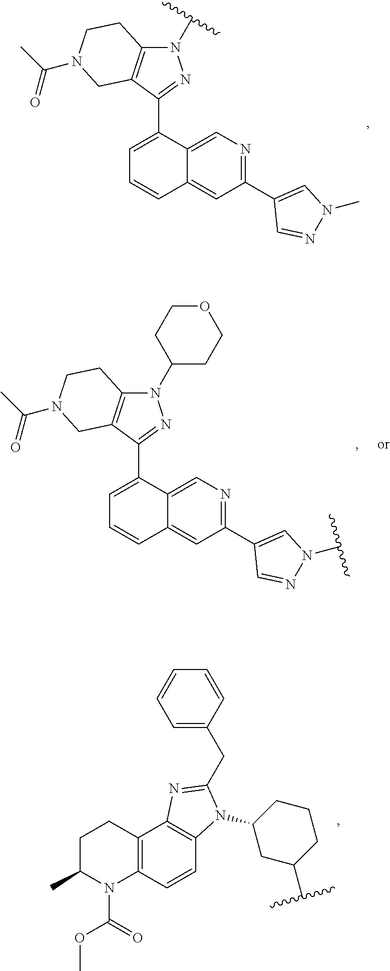

or a pharmaceutically acceptable salt or solvate thereof.

Embodiment 58. The compound of anyone of embodiments 9-14, or a pharmaceutically acceptable salt or solvate thereof, wherein SB-CBP/p300 has one of the following structures:

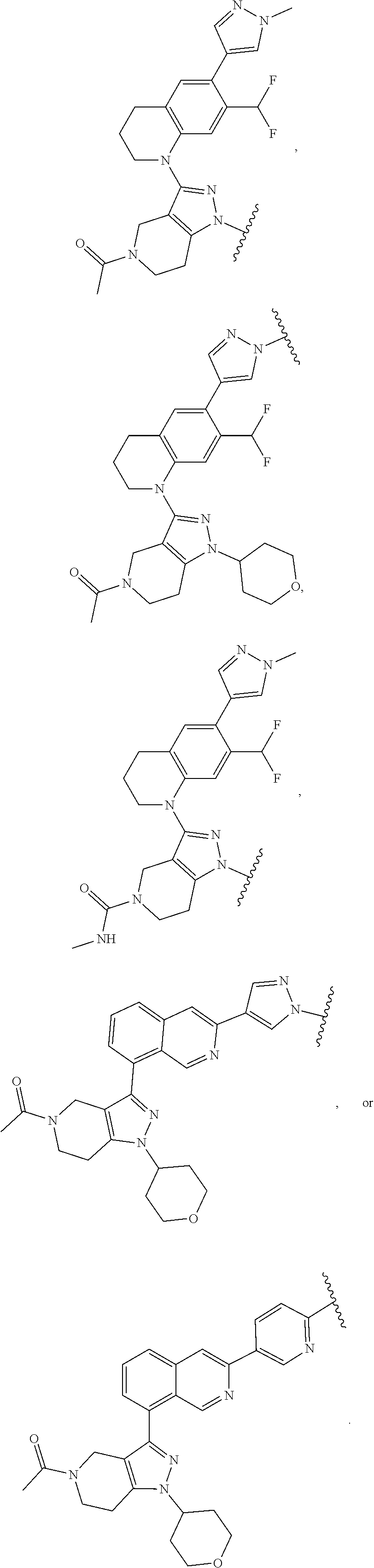

Embodiment 59. The compound of anyone of embodiments 9-14, or a pharmaceutically acceptable salt or solvate thereof, wherein SB-CBP/p300 has one of the following structures:

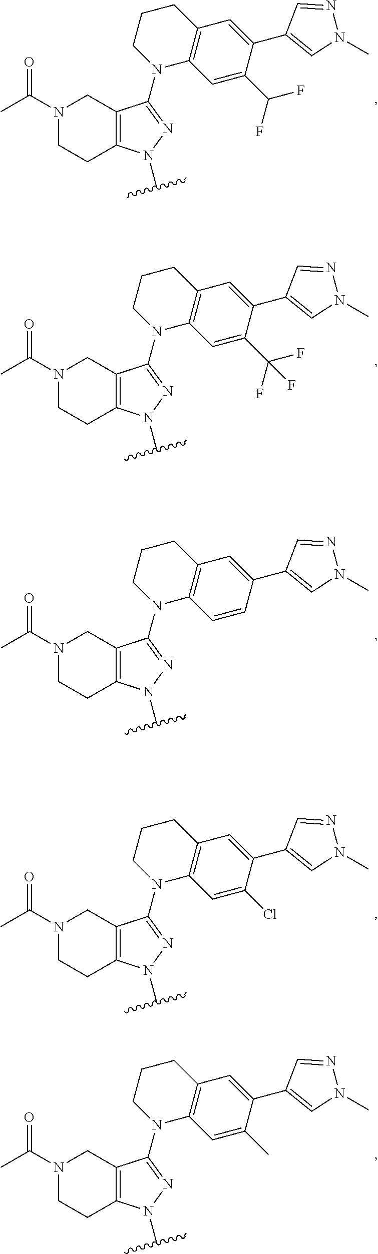

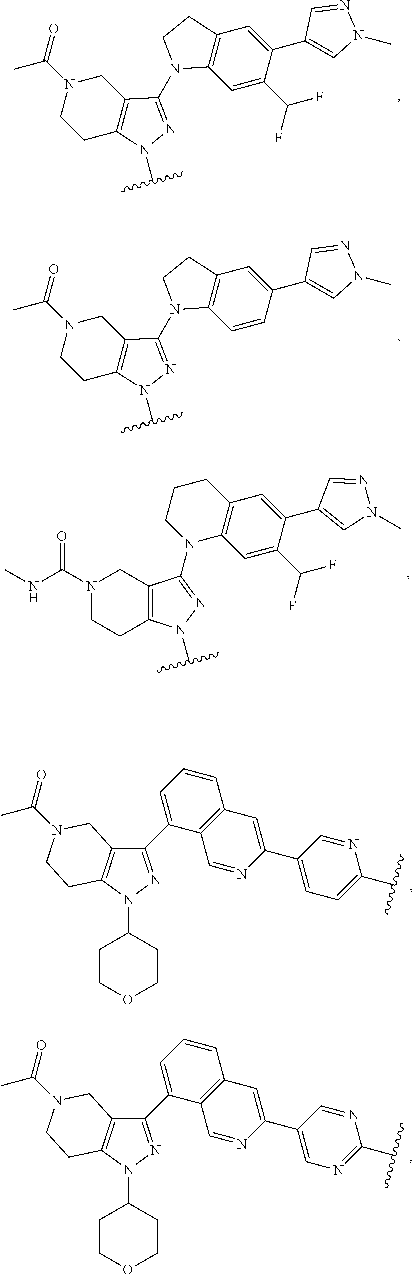

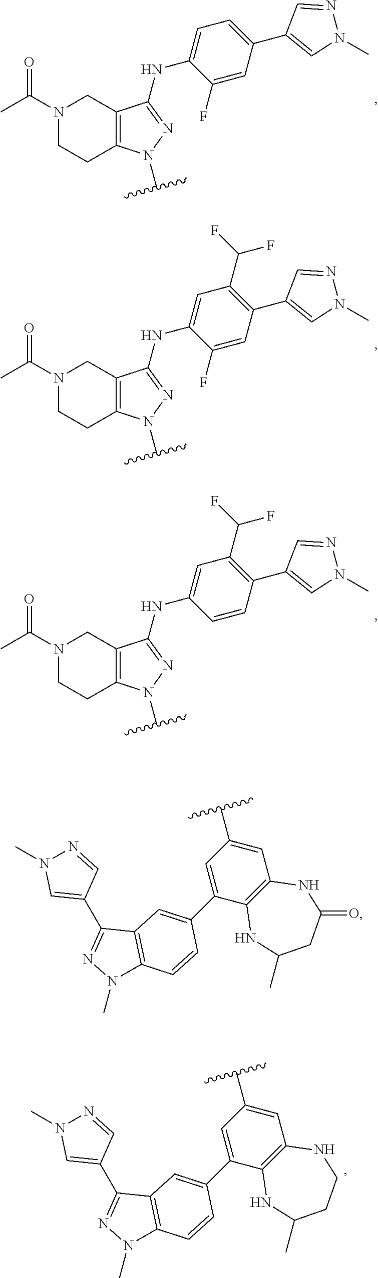

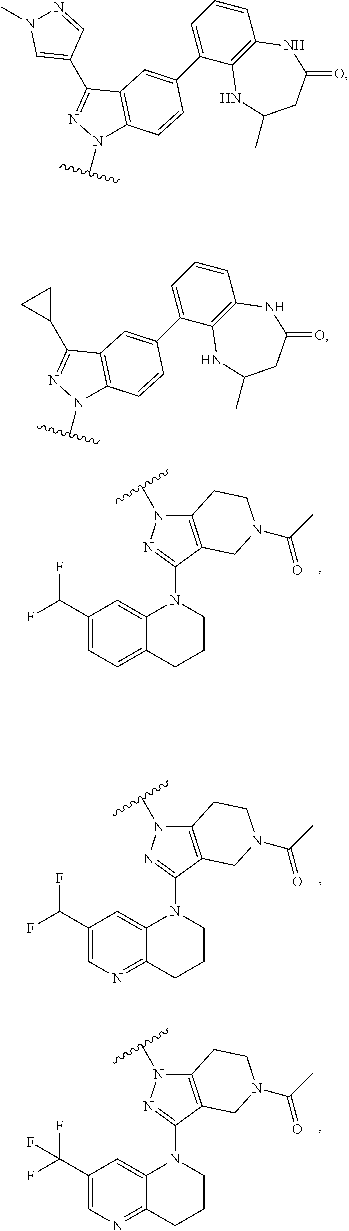 [SMILES: *n1nc(-c2cnn(C)c2)c2cc(-c3cccc4c3NC(C)CC(=O)N4)ccc21 ; *n1nc(C2CC2)c2cc(-c3cccc4c3NC(C)CC(=O)N4)ccc21 ; *n1nc(N2CCCc3ccc(C(F)F)cc32)c2c1CCN(C(C)=O)C2 ; *n1nc(N2CCCc3ncc(C(F)F)cc32)c2c1CCN(C(C)=O)C2 ; *n1nc(N2CCCc3ncc(C(F)(F)F)cc32)c2c1CCN(C(C)=O)C2]

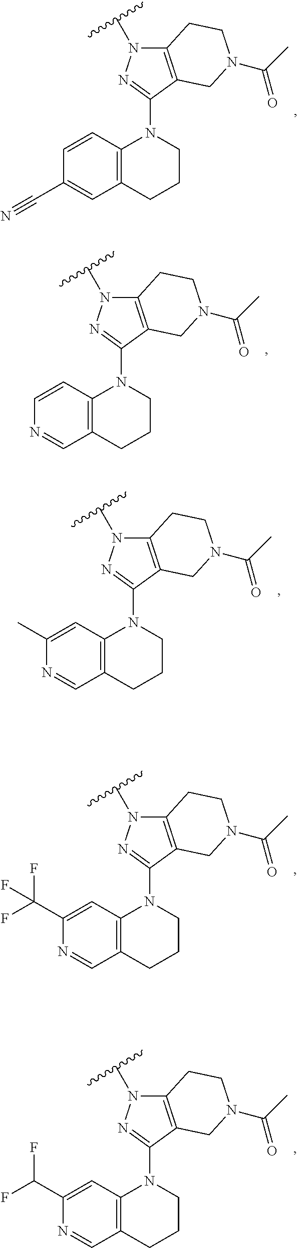

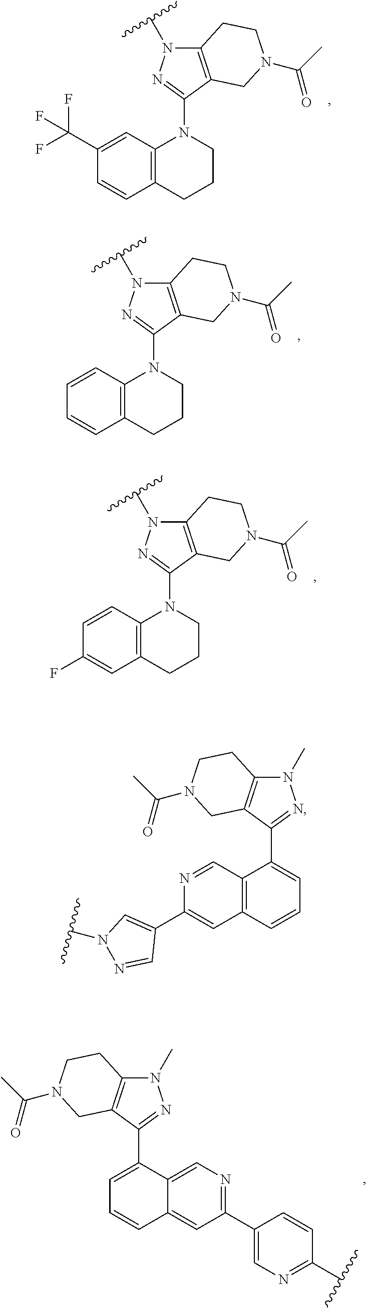

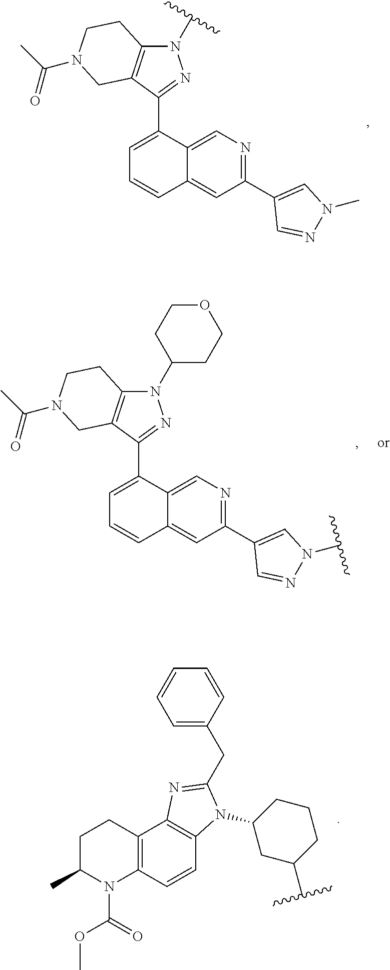

Embodiment 60. The compound of any one of embodiments 1-59, wherein the DP is a nuclear nuclear receptor (NR) transcription factor.

Embodiment 61. The compound of any one of embodiments 1-59, wherein the DP is a nuclear nuclear receptor (NR) transcription factor that is the androgen receptor (AR) and the BDP is a binder of AR (B-AR); or the DP is the estrogen receptor (ER) and the BDP is a binder of ER (B-ER); or retinoic acid receptor-related orphan nuclear receptor γ (RORγ) and the B-DP is a binder of RORγ (B-ROR).

Embodiment 62. The compound of any one of embodiments 1-59, wherein the DP is the androgen receptor (AR) and the BDP is a binder of AR (B-AR), wherein the B-AR is an AR antagonist, a selective androgen receptor modulator (SARM), or selective androgen receptor degrader (SARD); and the B-ER is an ER antagonist, a selective estrogen receptor modulator (SERM), or a selective estrogen receptor degrader (SERD); wherein the AR antagonist, SARM, or SARD is a non-steroidal AR ligand or a steroidal AR ligand; and wherein the ER antagonist, SERM, or SERD is a non-steroidal ER ligand or a steroidal ER ligand.

Embodiment 63. The compound of any one of embodiments 1-59, wherein the DP is the androgen receptor (AR) and the BDP is a binder of AR (B-AR) that is an AR antagonist, a selective androgen receptor modulator (SARM), or a selective androgen receptor degrader (SARD); wherein the AR antagonist, SARM, or SARD binds to the ligand-binding domain (LBD) of AR.

Embodiment 64. The compound of embodiment 63, wherein the AR antagonist, SARM, or SARD comprises:
a head moiety that occupies the LBD region of the AR; and 1) an optional core moiety; 2) an optional tail moiety covalently attached to the core moiety; or 3) both 1) and 2).

Embodiment 65. The compound of embodiment 64, wherein the head moiety of the AR antagonist, SARM, or SARD forms hydrogen bonds with the side chains of Gln 711 and Arg752 of the LBD of AR.

Embodiment 66. The compound of any one of embodiments 64-65, wherein:
the optional linker is covalently attached to the head moiety if the optional core moiety and optional tail moiety are absent; or
the optional linker is covalently attached to the core moiety if the optional tail moiety is absent; or
the optional linker is covalently attached to the tail moiety.

Embodiment 67. The compound of embodiment 63, wherein B-AR is flutamide, hydroxylflutamide, nilutamide, bicalutamide, enzalutamide, apalutamide, ODM201, AZD3514, or BMS641988.

Embodiment 68. The compound of any one of embodiments 64-66, wherein the head group is selected from:

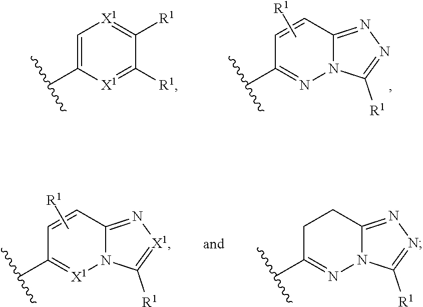

each X¹ is independently —CR¹— or —N—; and
each R¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted 3- to 8-membered heterocycloalkyl, —CN, —NO₂, —OH, —OR⁴, OC(═O)R⁴, —OC(═O)N(R⁵)₂, —OC(═O)OR⁵, —OC(═O)NR⁵, —SH, —SR⁴, —S(═O)R⁴, —S(═O)₂R⁵, —S(═O)₂OR⁴, —S(═O)₂N(R⁵)₂, —N(R⁵)₂, —NR⁵C(═O)NR⁵, —NR⁵C(═O)R⁴, —NR⁵C(═O)OR⁵, —NR⁵S(═O)₂R⁵, —C(═O)R⁵, —C(═O)OR⁵, or —C(═O)N(R⁵)₂.

Embodiment 69. The compound of any one of embodiments 64-66, wherein the head group is selected from:

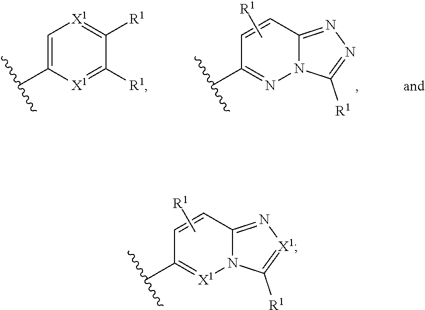

one X¹ is —CR¹— and the other X¹ is —CR¹— or —N—; and
each R¹ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, —OCH₂CH₃, —CN, —C(═O)NH₂ or —C(═O)NH(CH₃).

Embodiment 70. The compound of any one of embodiments 64-66, wherein the head group is selected from:

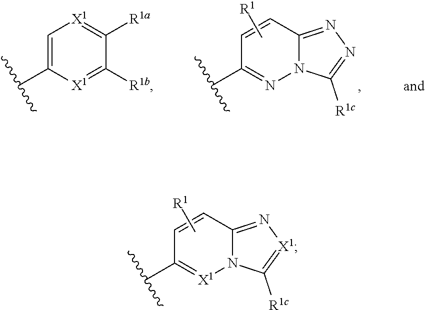

each X¹ is independently —CR¹— or —N—;
R^{1a} is —CN, —NO₂, —C(═O)R⁵, —C(═O)OR⁵, or —C(═O)N(R⁵)₂;
R^{1b} is hydrogen, halogen, C₁-C₄alkyl, C₁-C₄fluoroalkyl, —CN, —OH, —OR⁴, or —SR⁴;
R^{1c} is hydrogen, halogen, C₁-C₄alkyl, C₁-C₄fluoroalkyl, or —CN;
each R¹ is independently hydrogen, halogen, C₁-C₄alkyl, C₁-C₄fluoroalkyl, —CN, —OH, or —OR⁴;
each R⁴ is independently C₁-C₄alkyl, or C₁-C₄fluoroalkyl; and
each R⁵ is independently hydrogen, C₁-C₄alkyl, or C₁-C₄fluoroalkyl.

Embodiment 71. The compound of any one of embodiments 64-66, wherein the head group is selected from:

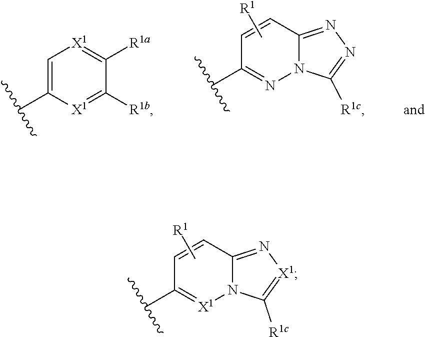

one X¹ is —CR¹— and the other X¹ is —CR¹— or —N—;
R^{1a} is —CN, —NO₂, —C(═O)NH₂ or —C(═O)NH(CH₃);
R^{1b} is hydrogen, F, Cl, Br, I, —CH₃, —CH₂CH₃, —CH₂F, —CHF₂, —CF₃, —OH, —OCF₃, —OCH₃, —OCH₂CH₃, or —CN;
each R^{1c} is hydrogen, F, Cl, Br, —CH₃, —CH₂F, —CHF₂, or —CF₃; and
each R^{1d} is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, or —OCH₂CH₃.

Embodiment 72. The compound of any one of embodiments 64-66, wherein the head group is selected from:

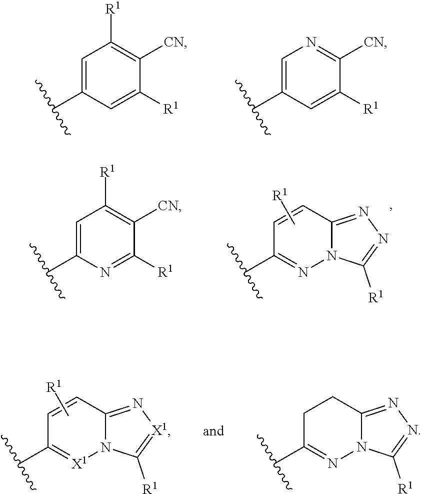

Embodiment 73. The compound of any one of embodiments 64-66, wherein the head group is selected from:

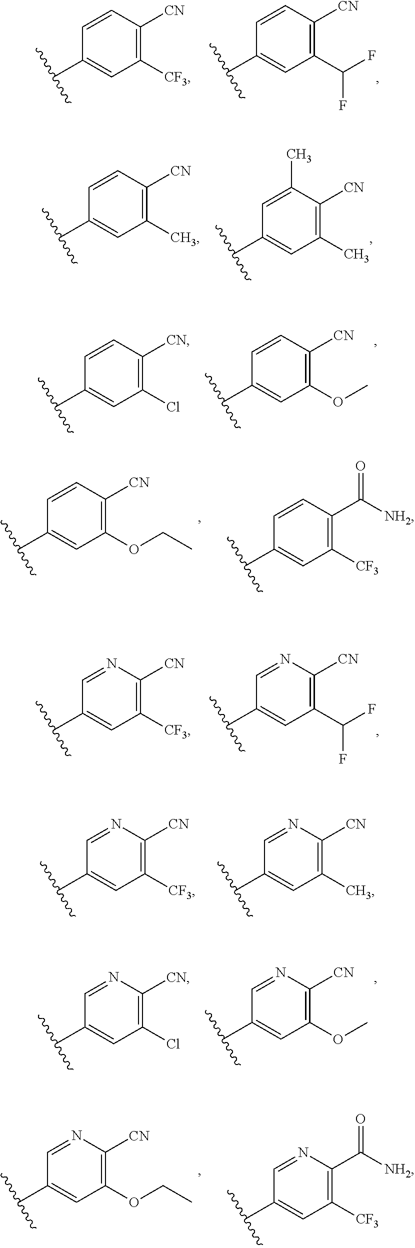

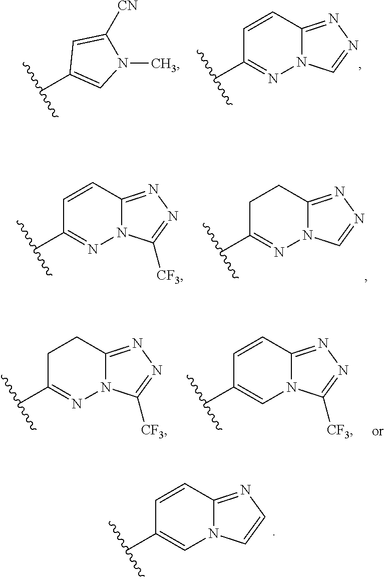

Embodiment 74. The compound of any one of embodiments 64-66 or 68-73, wherein the optional core comprises a group selected from:

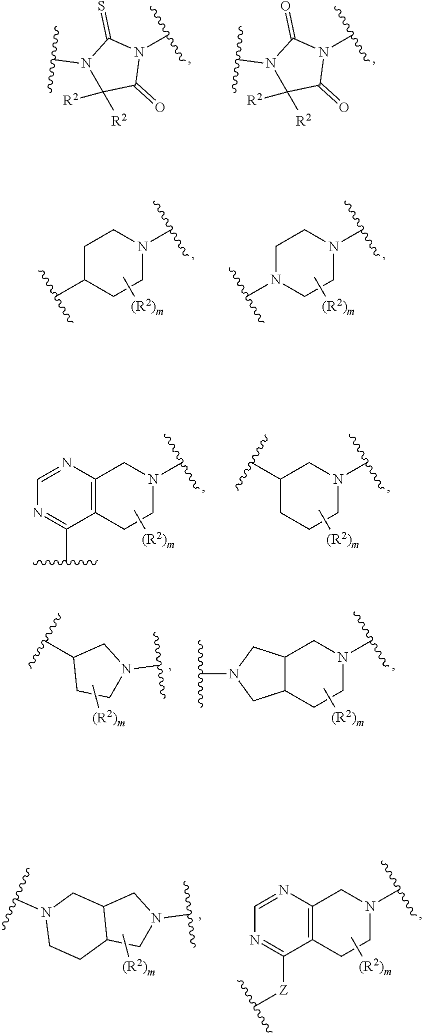

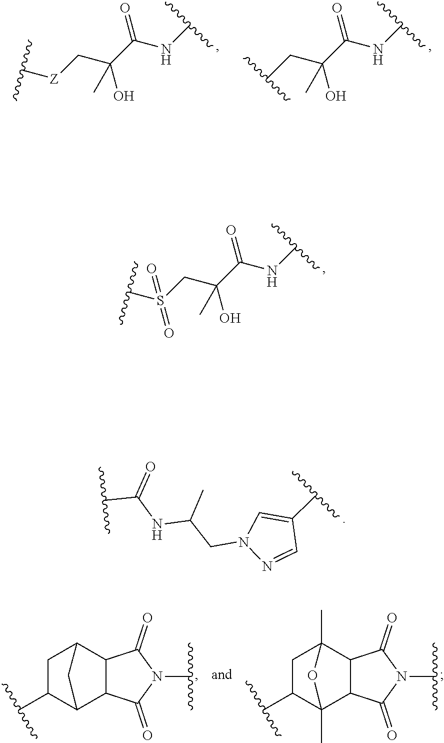

Z is —O— or —NR⁵—;
each R² is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR⁴, OC(═O)R⁴, —S(═O)R⁴, —S(═O)₂R⁵, —S(═O)₂N(R⁵)₂, —N(R⁵)₂, —NR⁵C(═O)NR⁵, —NR⁵C(═O)R⁴, —C(═O)R⁵, —C(═O)OR⁵, or —C(═O)N(R⁵)₂; or
two R² on the same carbon atom are taken together with the carbon atom to which they are attached to form a substituted or unsubstituted C₃-C₈cycloalkyl or a substituted or unsubstituted monocyclic 3- to 8-membered heterocycloalkyl;
m is 0, 1, 2, 3, or 4;
each s is independently 1, 2, or 3;
each R⁴ is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R⁵ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R⁵ on the same N atom are taken together with the N atom to which they are attached to form a substituted or unsubstituted N-containing heterocycle.

Embodiment 75. The compound of any one of embodiments 64-66 or 68-73, wherein the optional core and optional tail comprises a group selected from:

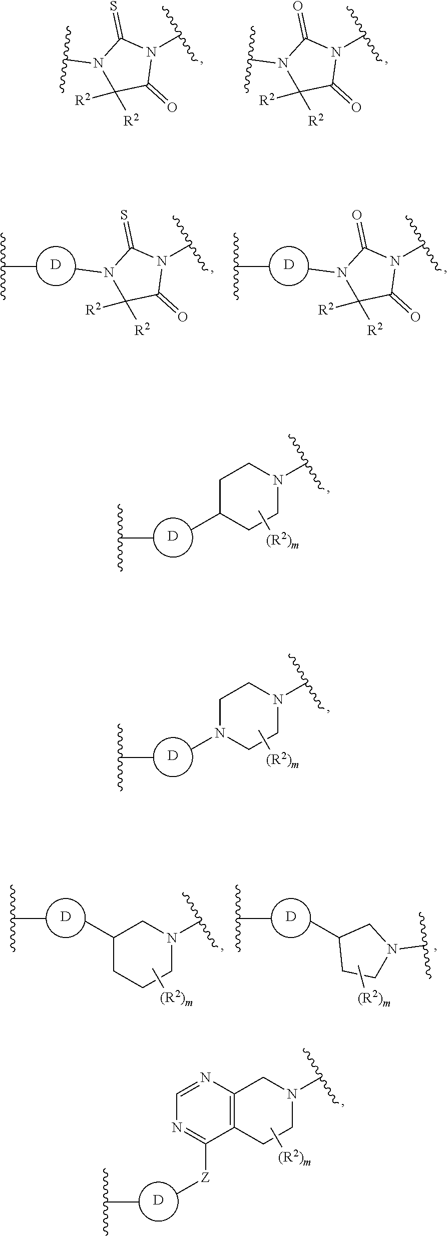

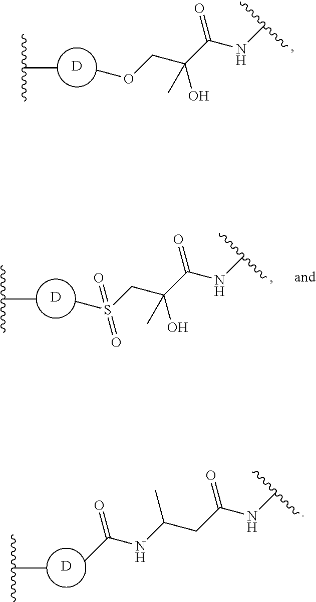

Embodiment 76. The compound of embodiment 75, wherein the optional tail comprises a ring D that is a 5-, 6-, 8-, 9- or 10-membered aryl or a 5-, 6-, 8-, 9- or 10-membered heteroaryl that is optionally substituted with s R³;
each R³ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂.

Embodiment 77. The compound of any one of embodiments 75-76, wherein ring D is phenyl, naphthyl, pyridinyl, pyrimidinyl, pyridazinyl, pyrazinyl, triazinyl, pyrrolyl, thiazolyl, oxazolyl, isoxazolyl, oxadiazolyl, imidazolyl, pyrazolyl, isothiazolyl, triazolyl, and tetrazolyl, wherein each ring D is optionally substituted substituted with s R³.

Embodiment 78. The compound of any one of embodiments 75-77, wherein ring D is

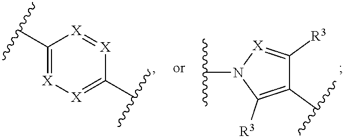

and each X is independently —CR³— or —N—.

Embodiment 79. The compound of any one of embodiments 75-77, wherein ring D is

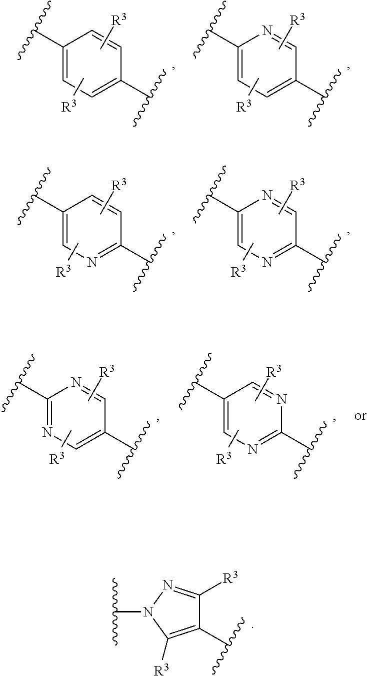

Embodiment 80. The compound of embodiment 62, wherein B-AR has one of the following structures:

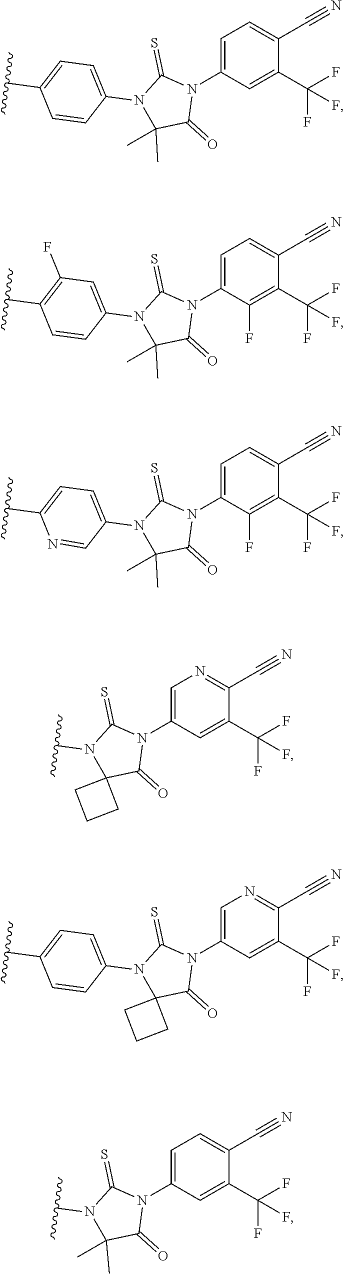 [SMILES: *c1ccc(N2C(=S)N(c3ccc(C#N)c(C(F)(F)F)c3)C(=O)C2(C)C)cc1 ; *c1ccc(N2C(=S)N(c3ccc(C#N)c(C(F)(F)F)c3F)C(=O)C2(C)C)cc1F ; *c1ccc(N2C(=S)N(c3ccc(C#N)c(C(F)(F)F)c3F)C(=O)C2(C)C)cn1 ; *N1C(=S)N(c2cnc(C#N)c(C(F)(F)F)c2)C(=O)C12CCC2 ; *c1ccc(N2C(=S)N(c3cnc(C#N)c(C(F)(F)F)c3)C(=O)C23CCC3)cc1 ; *N1C(=S)N(c2ccc(C#N)c(C(F)(F)F)c2)C(=O)C1(C)C]

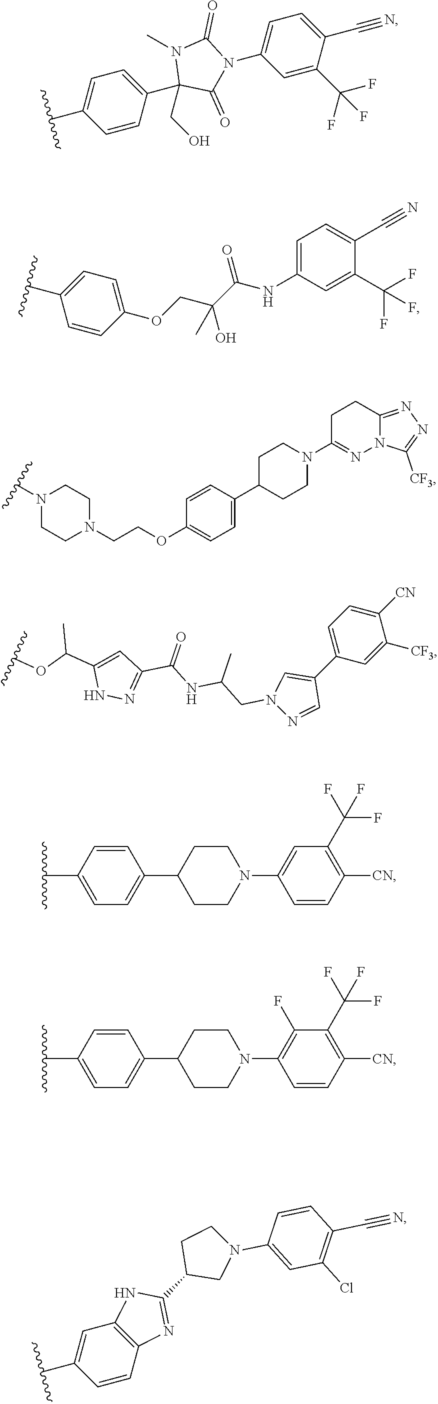

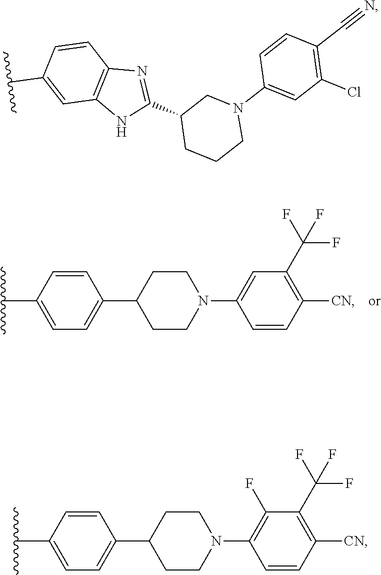 [SMILES: *c1ccc2nc([C@H]3CCCN(c4ccc(C#N)c(Cl)c4)C3)[nH]c2c1 ; *c1ccc(C2CCN(c3ccc(C#N)c(C(F)(F)F)c3)CC2)cc1 ; *c1ccc(C2CCN(c3ccc(C#N)c(C(F)(F)F)c3F)CC2)cc1]

or a pharmaceutically acceptable salt or solvate thereof.

Embodiment 81. The compound of any one of embodiments 1-59, wherein the DP is the estrogen receptor (ER) and the BDP is a binder of ER (B-ER) that is an ER antagonist, a selective estrogen receptor modulator (SERM), or a selective estrogen receptor degrader (SERD).

Embodiment 82. The compound of embodiment 81, wherein the B-ER binds to the ligand-binding domain (LBD) of ER.

Embodiment 83. The compound of any one of embodiments 81-82, wherein the B-ER is:

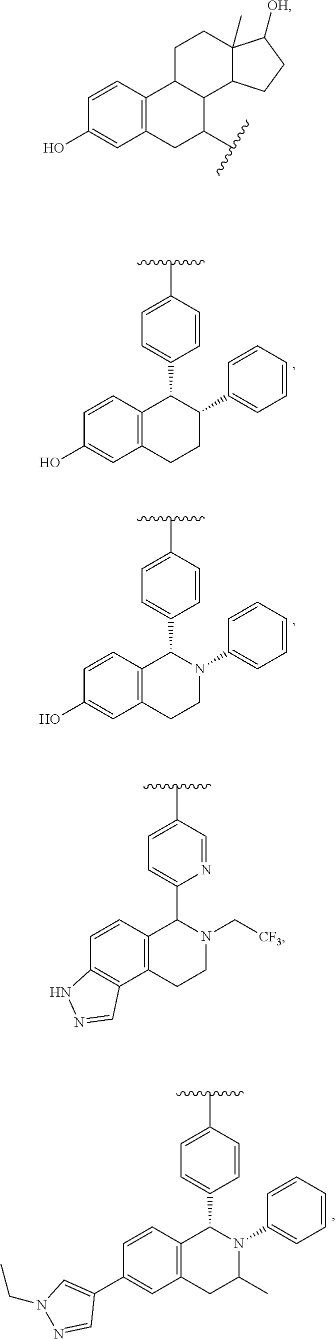

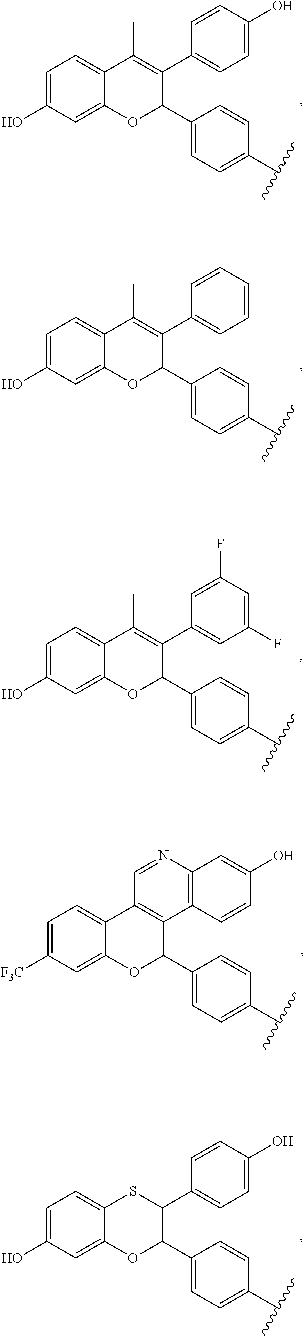 [SMILES: *c1ccc(C2Oc3cc(O)ccc3C(C)=C2c2ccc(O)cc2)cc1 ; *c1ccc(C2Oc3cc(O)ccc3C(C)=C2c2ccccc2)cc1 ; *c1ccc(C2Oc3cc(O)ccc3C(C)=C2c2cc(F)cc(F)c2)cc1 ; *c1ccc(C2Oc3cc(C(F)(F)F)ccc3-c3cnc4cc(O)ccc4c32)cc1 ; *c1ccc(C2Oc3cc(O)ccc3SC2c2ccc(O)cc2)cc1]

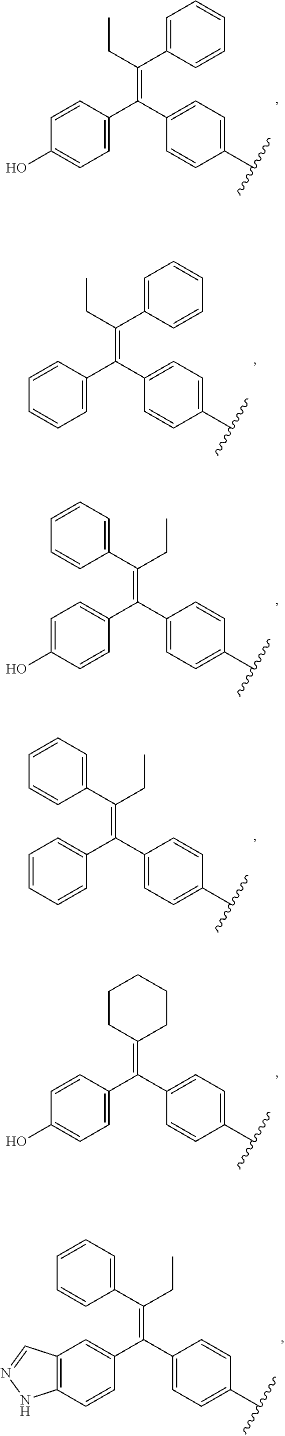 [SMILES: *c1ccc(/C(=C(/CC)c2ccccc2)c2ccc(O)cc2)cc1 ; *c1ccc(/C(=C(/CC)c2ccccc2)c2ccccc2)cc1 ; *c1ccc(/C(=C(\CC)c2ccccc2)c2ccc(O)cc2)cc1 ; *c1ccc(/C(=C(\CC)c2ccccc2)c2ccccc2)cc1 ; *c1ccc(C(=C2CCCCC2)c2ccc(O)cc2)cc1 ; *c1ccc(/C(=C(\CC)c2ccccc2)c2ccc3[nH]ncc3c2)cc1]

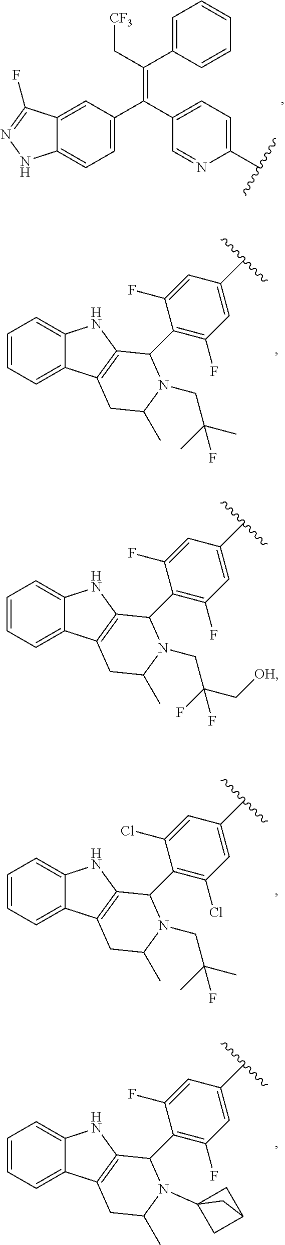 [SMILES: *c1ccc(/C(=C(/CC(F)(F)F)c2ccccc2)c2ccc3[nH]nc(F)c3c2)cn1 ; *c1cc(F)c(C2c3[nH]c4ccccc4c3CC(C)N2CC(C)(C)F)c(F)c1 ; *c1cc(F)c(C2c3[nH]c4ccccc4c3CC(C)N2CC(F)(F)CO)c(F)c1 ; *c1cc(Cl)c(C2c3[nH]c4ccccc4c3CC(C)N2CC(C)(C)F)c(Cl)c1 ; *c1cc(F)c(C2c3[nH]c4ccccc4c3CC(C)N2C23CC(C2)C3)c(F)c1]

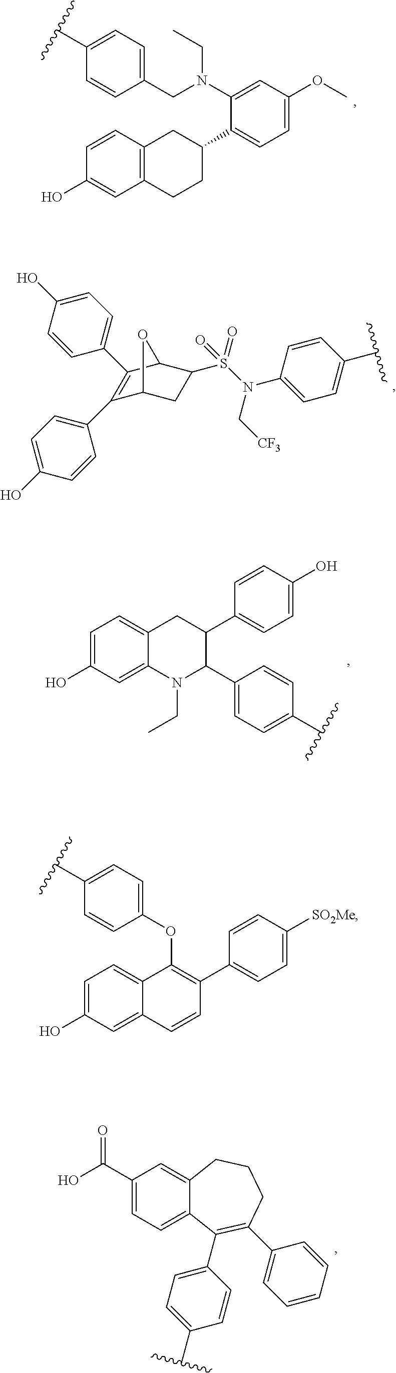 [SMILES: *c1ccc(CN(CC)c2cc(OC)ccc2[C@@H]2CCc3cc(O)ccc3C2)cc1 ; *c1ccc(N(CC(F)(F)F)S(=O)(=O)C2CC3OC2C(c2ccc(O)cc2)=C3c2ccc(O)cc2)cc1 ; *c1ccc(C2C(c3ccc(O)cc3)Cc3ccc(O)cc3N2CC)cc1 ; *c1ccc(Oc2c(-c3ccc(S(C)(=O)=O)cc3)ccc3cc(O)ccc23)cc1 ; *c1ccc(C2=C(c3ccccc3)CCCc3cc(C(=O)O)ccc32)cc1]

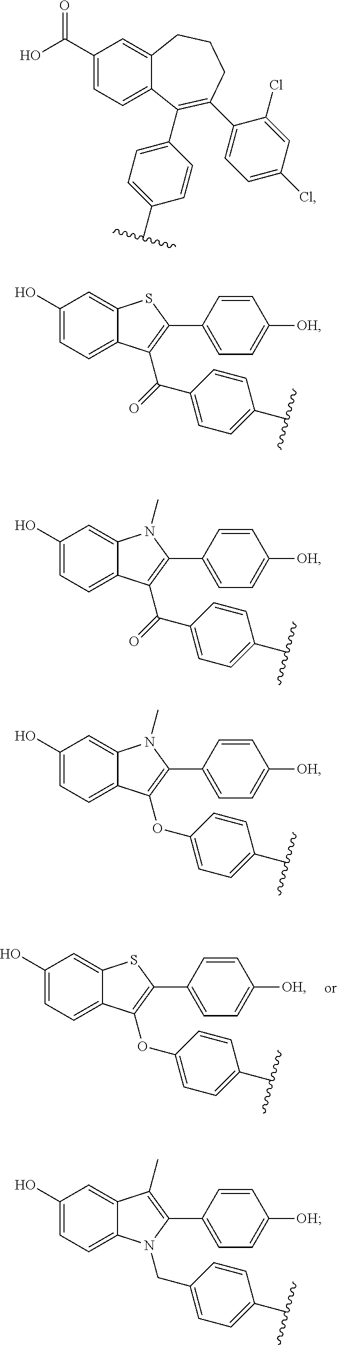 [SMILES: *c1ccc(C2=C(c3ccc(Cl)cc3Cl)CCCc3cc(C(=O)O)ccc32)cc1 ; *c1ccc(C(=O)c2c(-c3ccc(O)cc3)sc3cc(O)ccc23)cc1 ; *c1ccc(C(=O)c2c(-c3ccc(O)cc3)n(C)c3cc(O)ccc23)cc1 ; *c1ccc(Oc2c(-c3ccc(O)cc3)n(C)c3cc(O)ccc23)cc1 ; *c1ccc(Oc2c(-c3ccc(O)cc3)sc3cc(O)ccc23)cc1 ; *c1ccc(Cn2c(-c3ccc(O)cc3)c(C)c3cc(O)ccc32)cc1]

or a pharmaceutically acceptable salt or solvate thereof.

Embodiment 84. The compound of any one of embodiments 1-59, wherein the DP is chymase and the B-DP is a chymase inhibitor.

Embodiment 85. The compound of embodiment 84, wherein the chymase inhibitor is selective for chymase over cathepsin G.

Embodiment 86. The compound of embodiment 84 or 85, wherein the chymase inhibitor binds to Ser195 in the active cleft of chymase, binds to the S1 pocket of chymase, or both.

Embodiment 87. The compound of any one of embodiments 84-86, wherein the chymase inhibitor is HY-109059, Fulacimstat, BAY1142524, HY-12370, TY-51469, HY-100269, Chymase-IN-1, HY-12514, NK3201, NK3201, HY-122161, JNJ-10311795, RWJ-355871, JNJ-10311795 (RWJ-355871), HY-126988, TPC-806, SP-Chymostatin B, or analog thereof.

Embodiment 88. The compound of any one of embodiments 84-86, wherein the chymase inhibitor has one of the following structures:

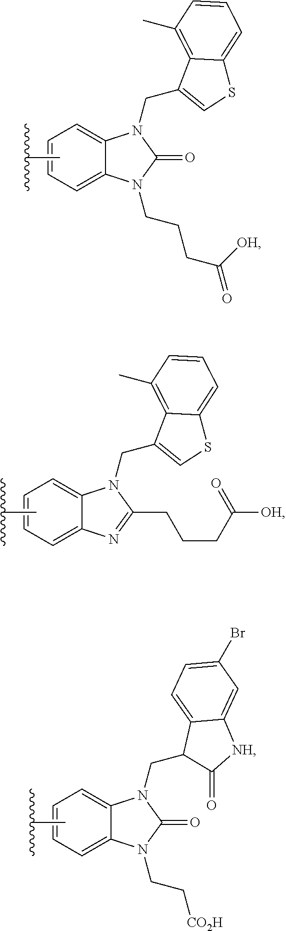

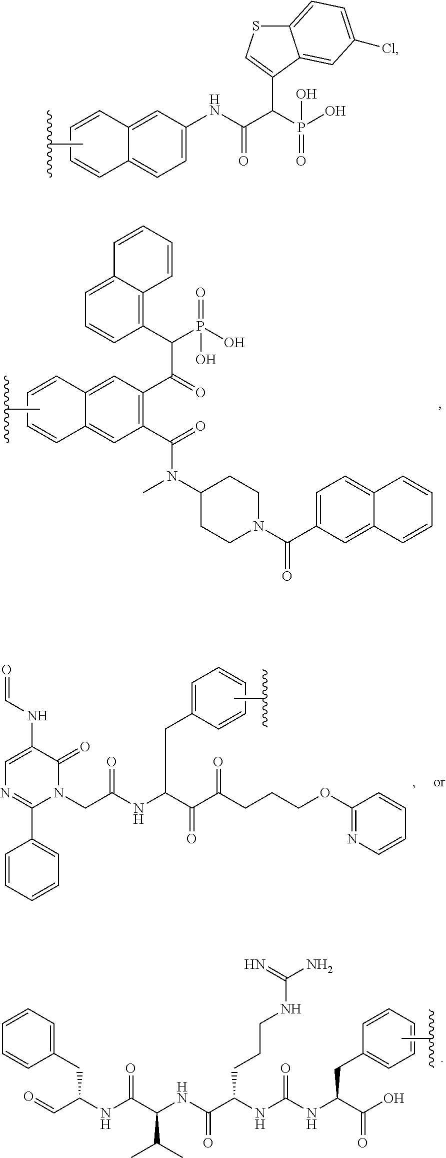

Embodiment 89. The compound of any one of embodiments 1-59, wherein the DP is retinoic acid receptor-related orphan nuclear receptor γ (RORγ) and the B-DP is a binder of RORγ (B-ROR).

Embodiment 90. The compound of embodiment 89, wherein the B-ROR is a RORγ antagonist, RORγ inverse agonist, RORγ inhibitor, or RORγ agonist.

Embodiment 91. The compound of embodiment 89 or 90, wherein the B-ROR binds to the ligand-binding domain (LBD) of RORγ.

Embodiment 92. The compound of any one of embodiments 89-91, wherein the B-ROR is S18-000003, A213, SHR168442, TMP778, BMS-986251, BMS-986313, A-9758, Digoxin, FC99, JNJ-61803534, SR2211, Ursolic acid, SR1001, TAK-828F, JNJ-61803534, JTE-451 (Retezorogant), BI 730357 (Bevurogant), ABBV-157 (Cedirogant), RTA-1701, AUR101, GSK2981278, PF-06763809, VTP-43742 (Vimirogant), AZD0284, GSK805, VPR-254, B1119, CQMU152, BIX119, VTP-938, or analog thereof.

Embodiment 93. The compound of any one of embodiments 89-91, wherein the B-ROR has one of the following structures:

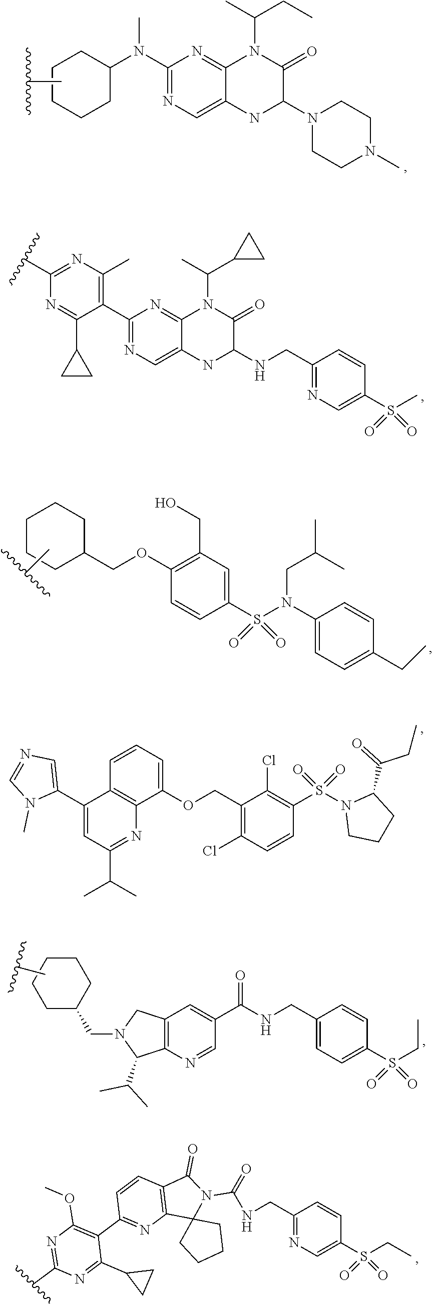

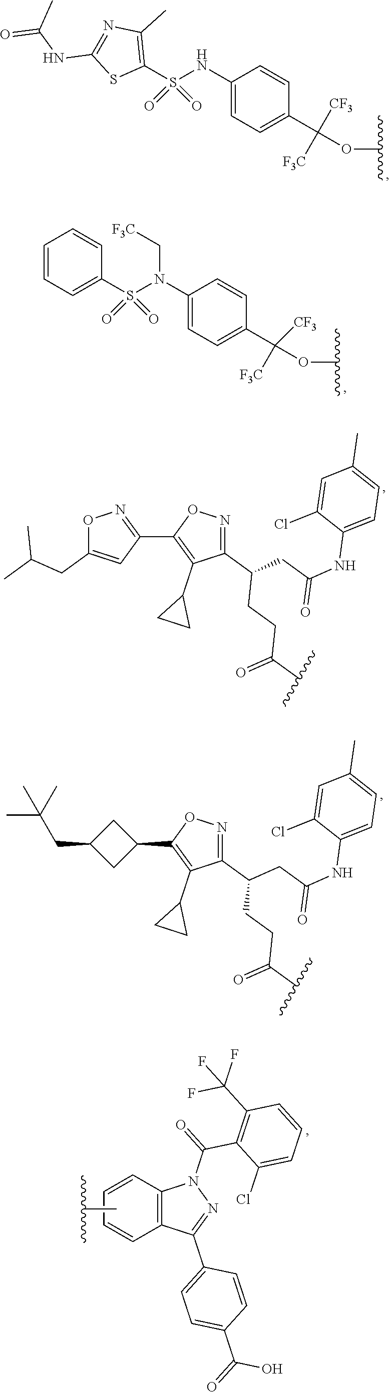

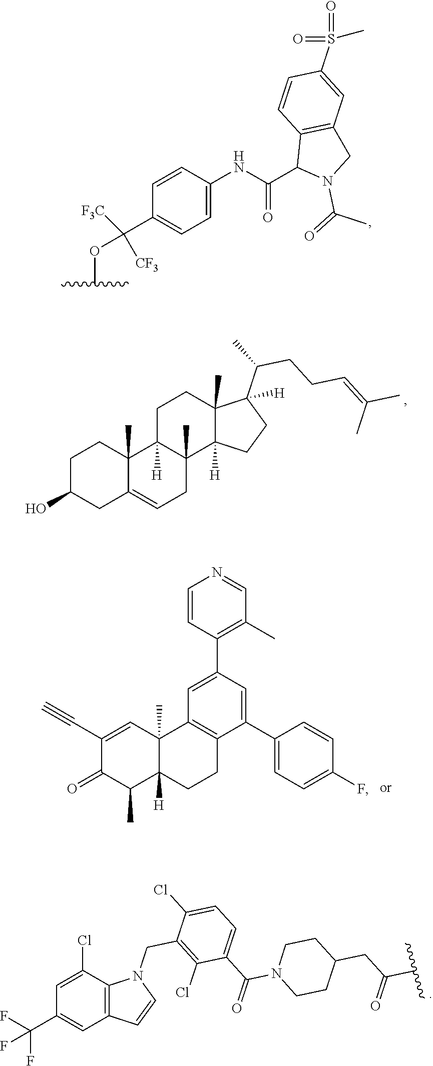

Embodiment 94. The compound of any one of embodiments 1-59, wherein the DP is indoleamine 2,3-dioxygenase 1 (IDO1) and the B-DP is a IDO1 inhibitor.

Embodiment 95. The compound of embodiment 94, wherein the B-DP is a IDO1 inhibitor that has one of the following structures:

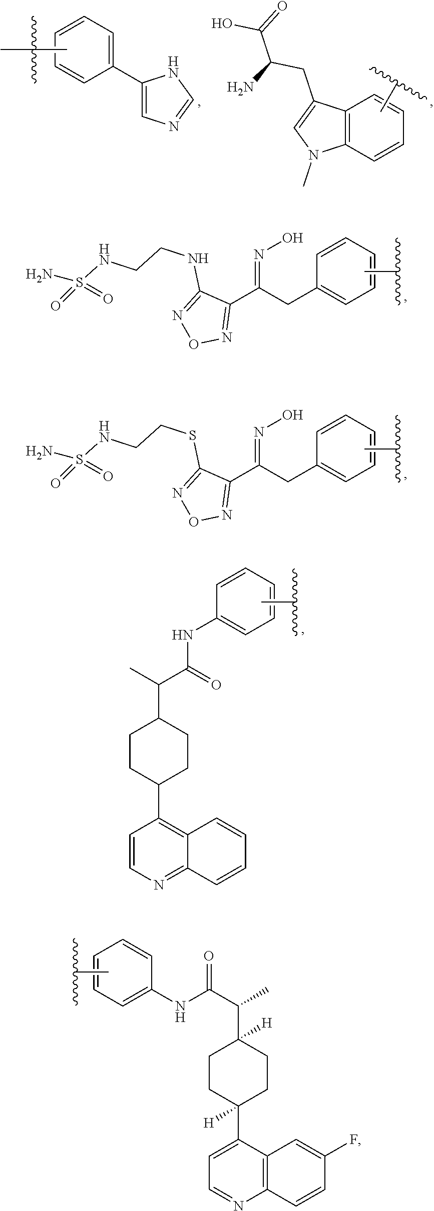

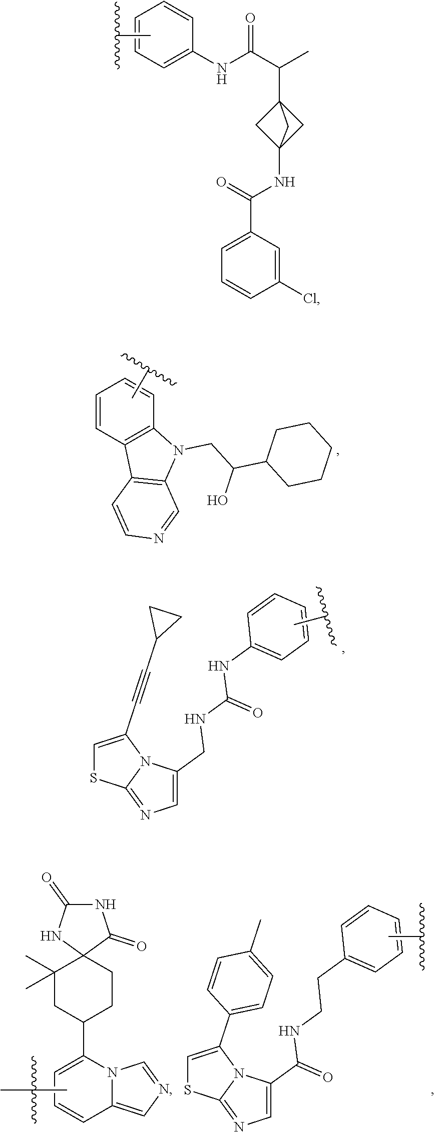

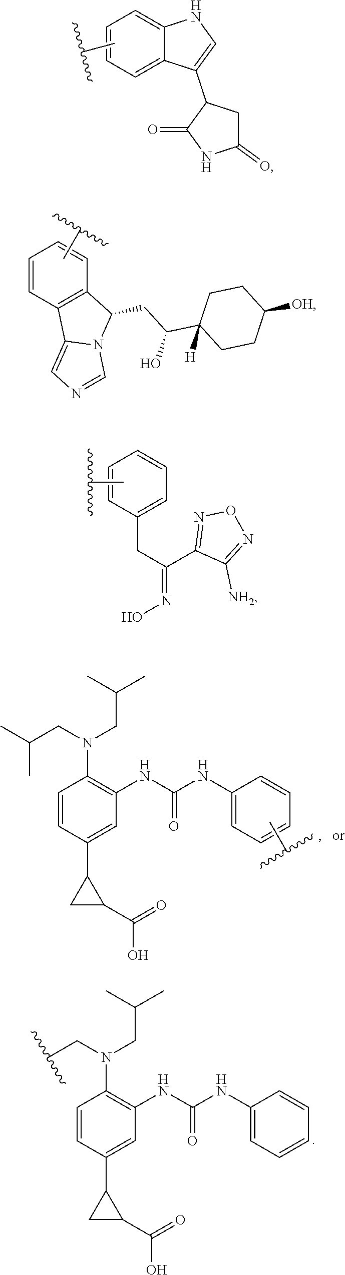

Embodiment 96. The compound of any one of embodiments 1-95, or a pharmaceutically acceptable salt or solvate thereof, wherein L is absent.

Embodiment 97. The compound of any one of embodiments 1-95, or a pharmaceutically acceptable salt or solvate thereof, wherein L comprises substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted 3- to 10-membered heterocycloalkyl, substituted or unsubstituted aryl, substituted or unsubstituted heteroaryl, or combinations thereof.

Embodiment 98. The compound of any one of embodiments 1-78, or a pharmaceutically acceptable salt or solvate thereof, wherein L is absent or

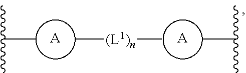

wherein:
each A is independently absent, substituted or unsubstituted monocyclic C₃-C₁₀cycloalkyl, substituted or unsubstituted bridged bicyclic C₃-C₁₀cycloalkyl, substituted or unsubstituted fused bicyclic C₃-C₁₀cycloalkyl, substituted or unsubstituted spiro bicyclic C₃-C₁₀cycloalkyl, substituted or unsubstituted monocyclic 3- to 10-membered heterocycloalkyl, substituted or unsubstituted bridged bicyclic 3- to 10-membered heterocycloalkyl, substituted or unsubstituted fused bicyclic 3- to 10-membered heterocycloalkyl, substituted or unsubstituted spiro bicyclic 3- to 10-membered heterocycloalkyl, substituted or unsubstituted aryl, or substituted or unsubstituted heteroaryl, wherein each A is independently unsubstituted or substituted with x R^{2b};
each L¹ is independently absent,

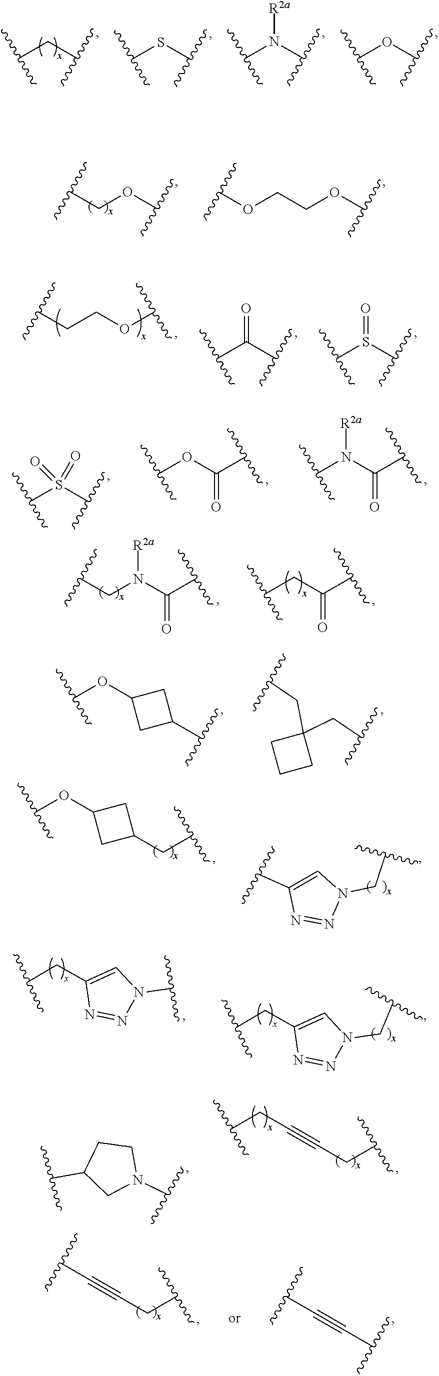

wherein each L¹ is independently unsubstituted or substituted with x R^{2b};
n is 1, 2, 3, 4, 5, or 6;
each x is independently 1, 2, 3, 4, 5, 6, 7, or 8;
each R^{2a} is independently hydrogen or substituted or unsubstituted C₁-C₆alkyl; and
each R^{2b} is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, —OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b});
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl; and
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

Embodiment 99. The compound of embodiment 98, or a pharmaceutically acceptable salt or solvate thereof, wherein:
each A is independently absent,

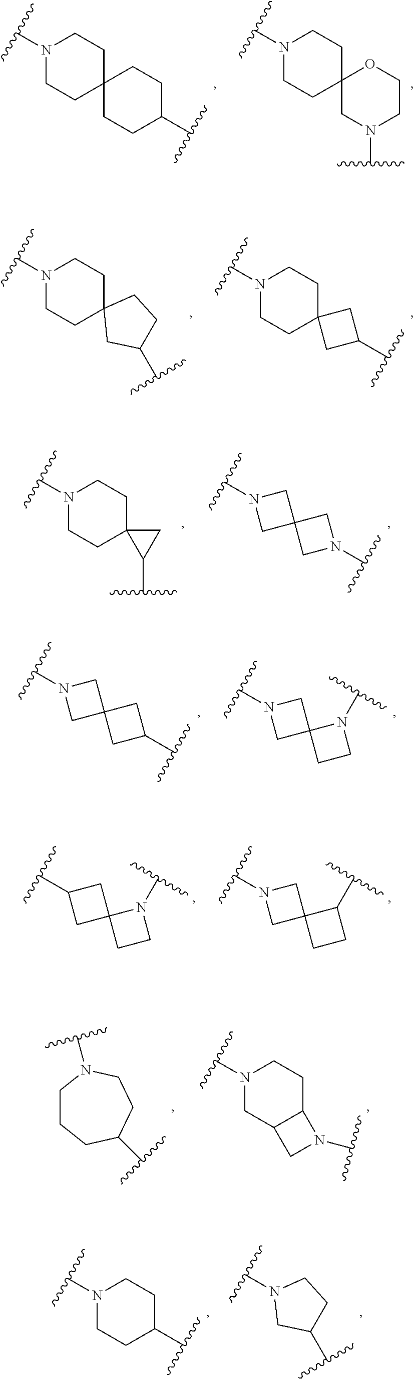

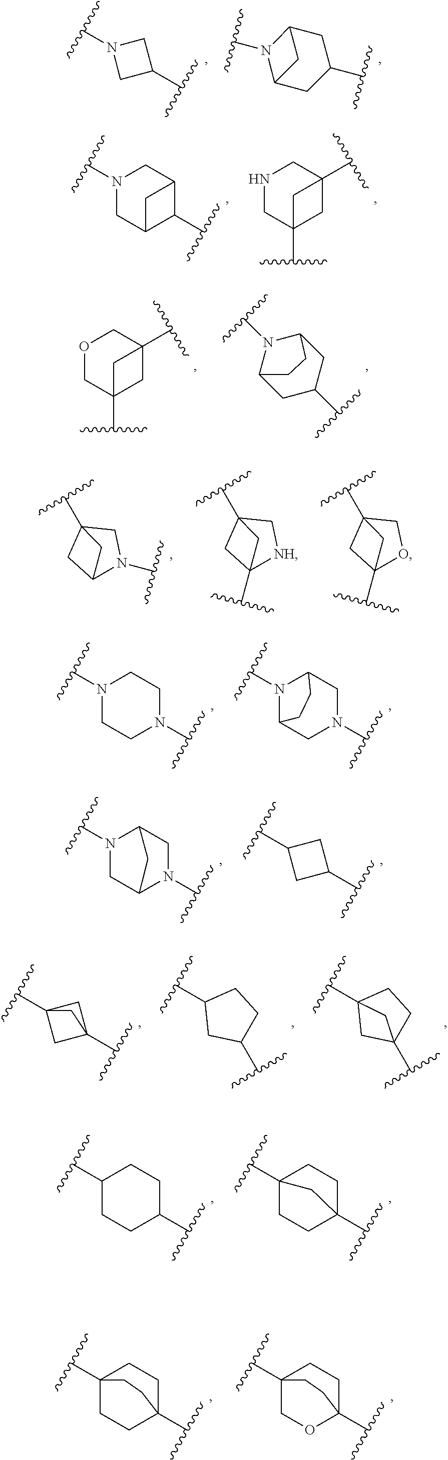

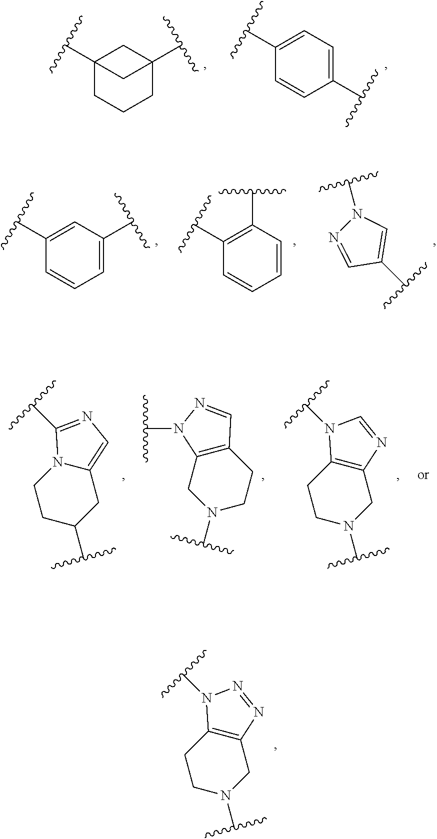

wherein each A is independently unsubstituted or substituted with x R^{2b}; and
(L¹)ₙ is absent,

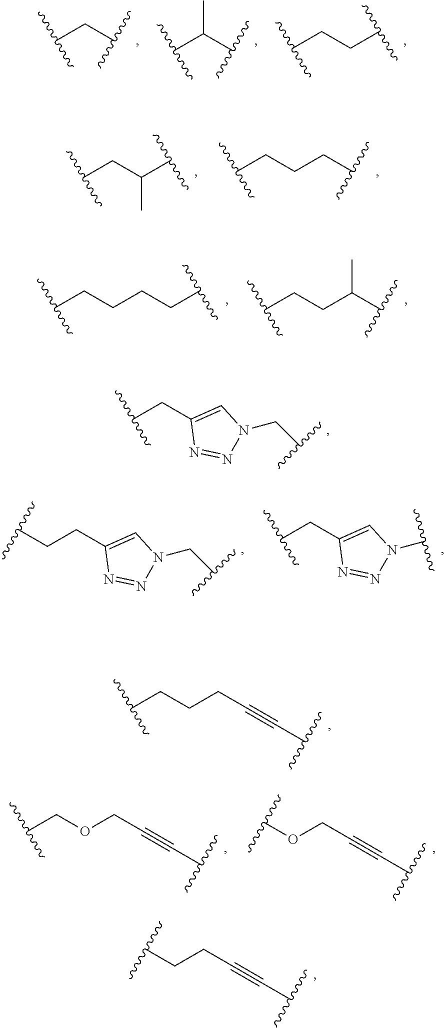

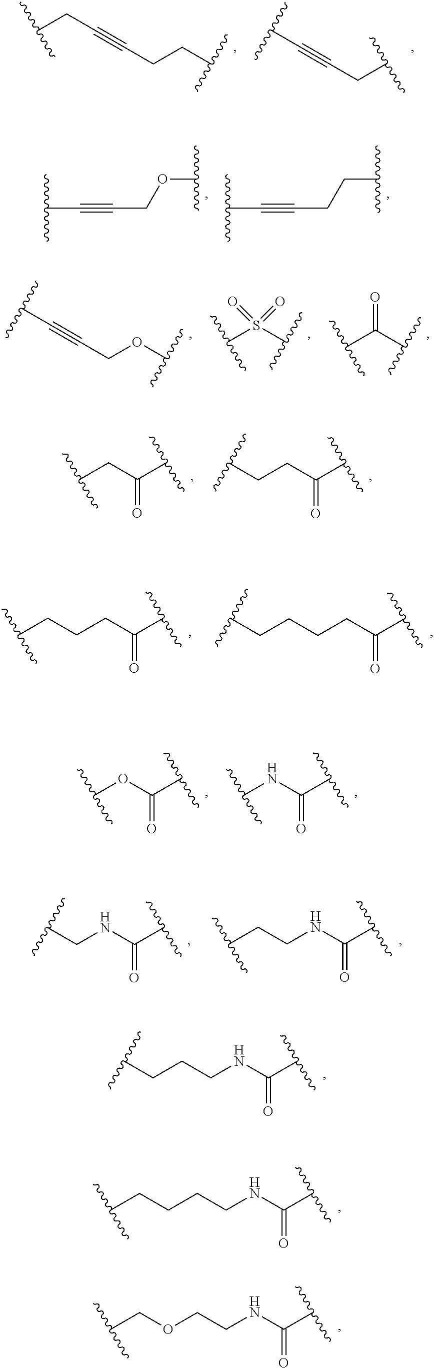

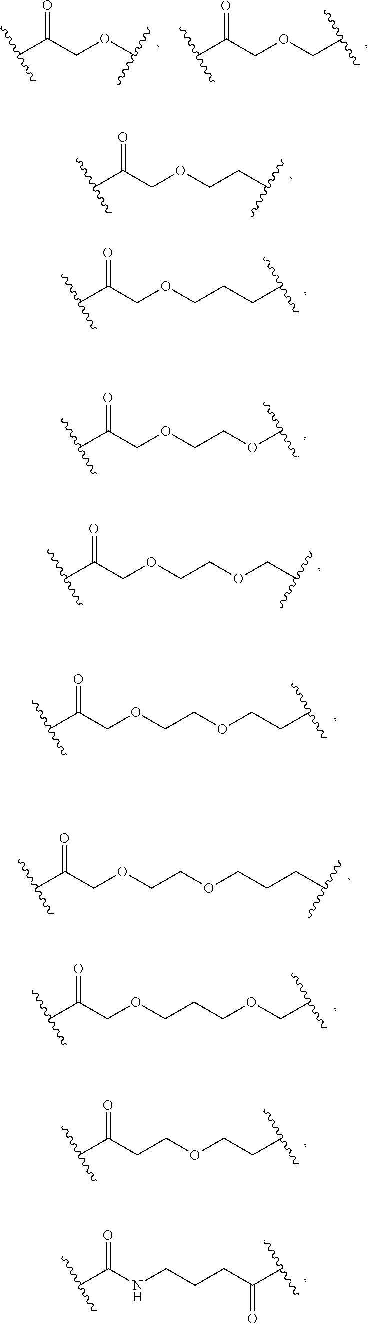

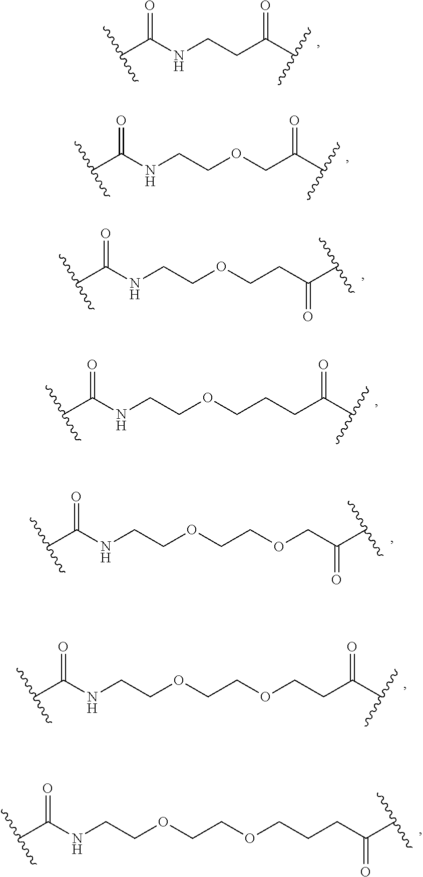

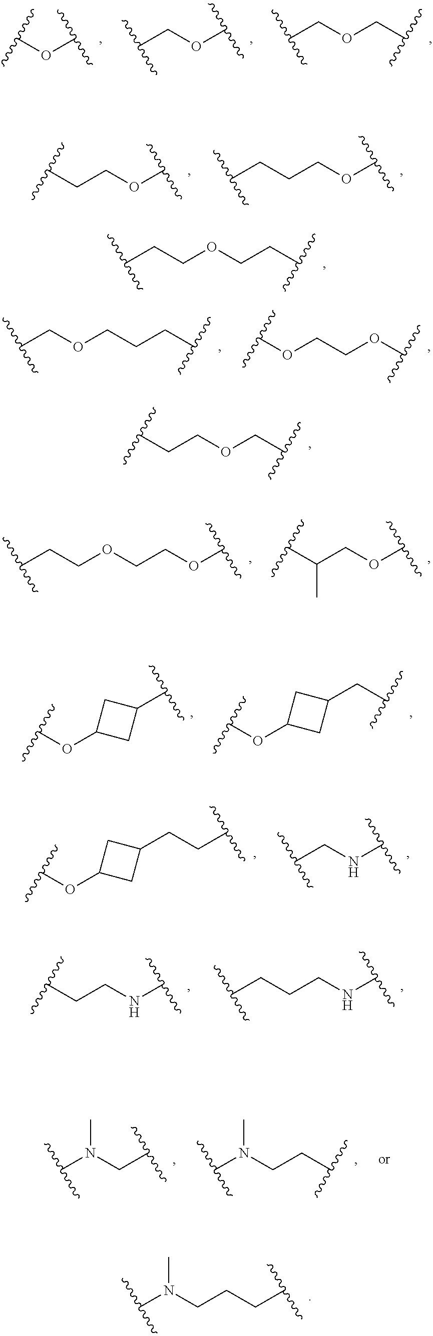

Embodiment 100. The compound of any one of embodiments 1-95, or a pharmaceutically acceptable salt or solvate thereof, wherein L is absent or

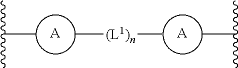

wherein:
each A is independently absent,

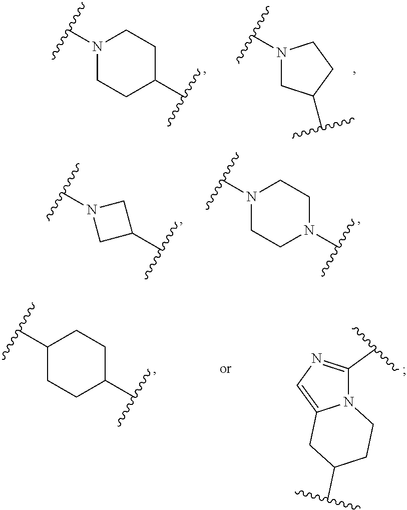 [SMILES: *C1CCN(*)CC1 ; *C1CCN(*)C1 ; *C1CN(*)C1 ; *N1CCN(*)CC1 ; *C1CCC(*)CC1 ; *c1ncc2n1CCC(*)C2]

each L¹ is independently absent,

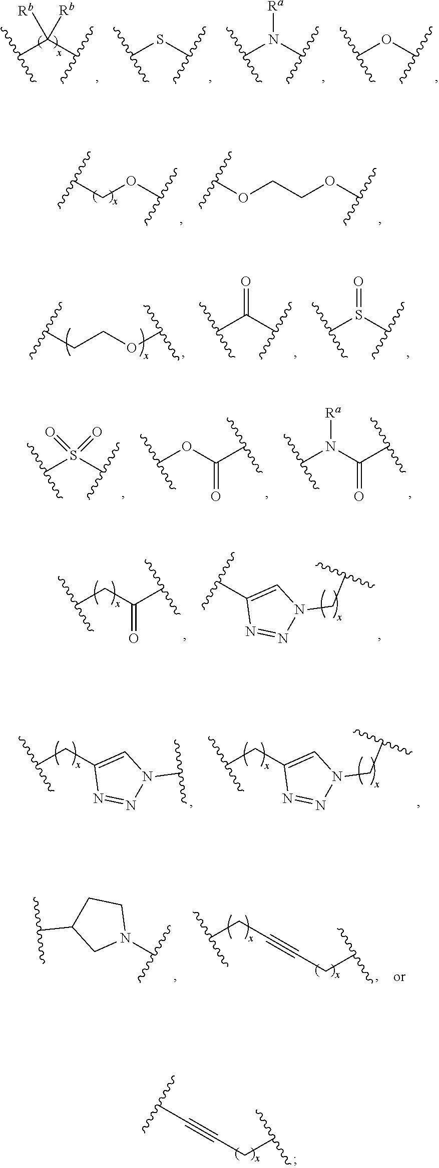

n is 1, 2, or 3; each x is independently 1, 2, 3, 4, 5, or 6;
each R^{a} is independently hydrogen or substituted or unsubstituted C₁-C₆alkyl; and
each R^{b} is independently hydrogen, halogen, or substituted or unsubstituted C₁-C₆alkyl.

Embodiment 101. The compound of any one of embodiments 98-100, or a pharmaceutically acceptable salt or solvate thereof, wherein:
(L¹)ₙ is absent,

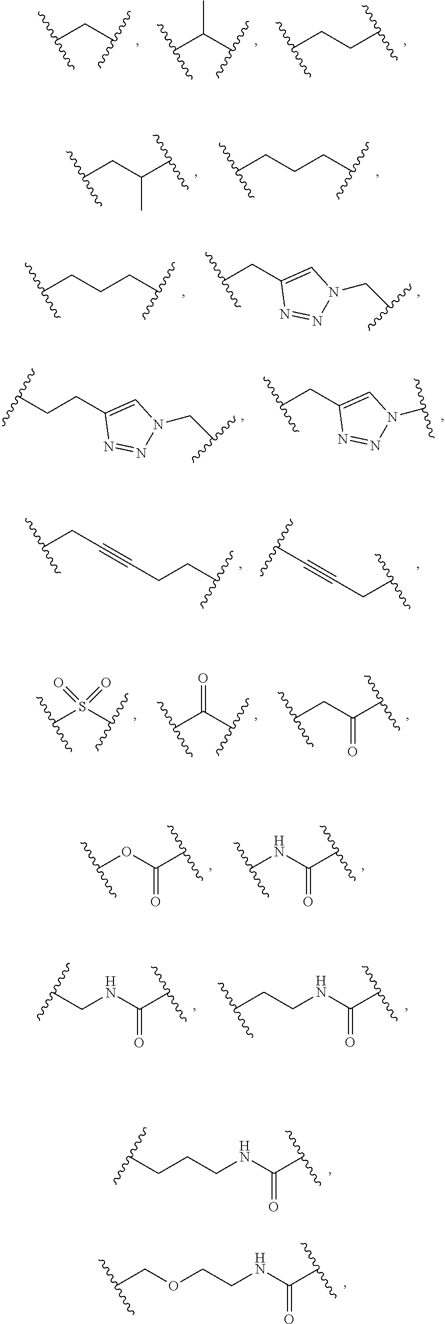

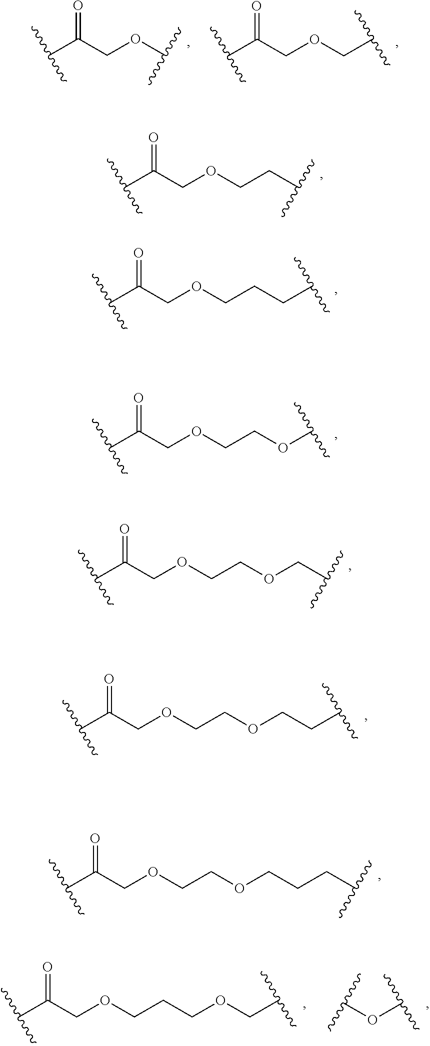

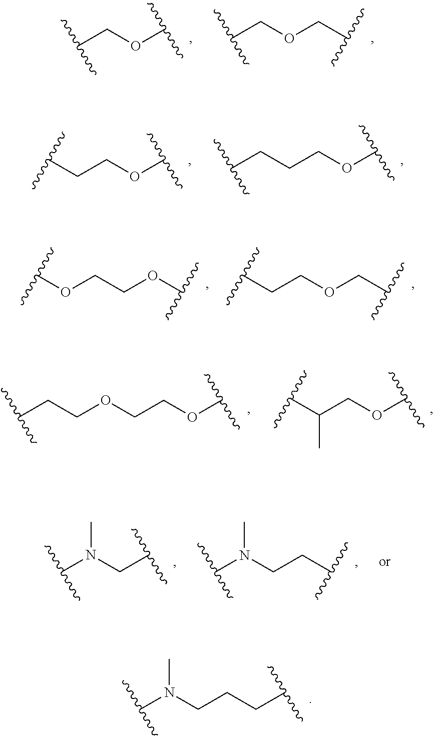

Embodiment 102. The compound of any one of embodiments 1-95, or a pharmaceutically acceptable salt or solvate thereof, wherein L has one of the following structures:

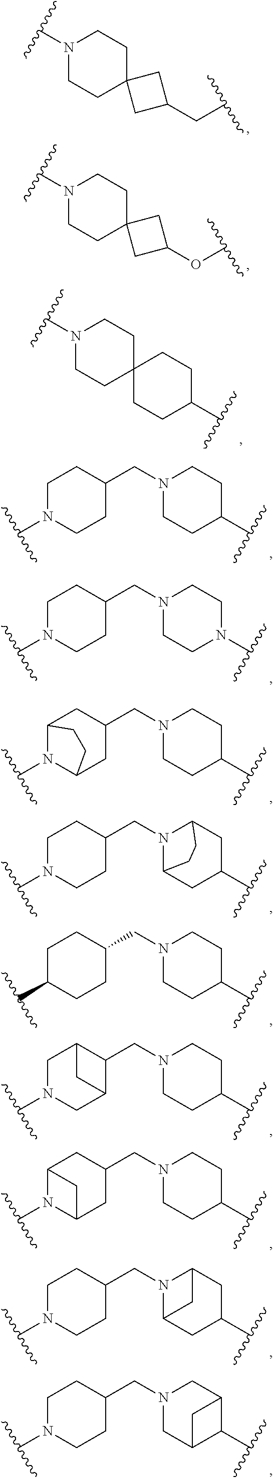

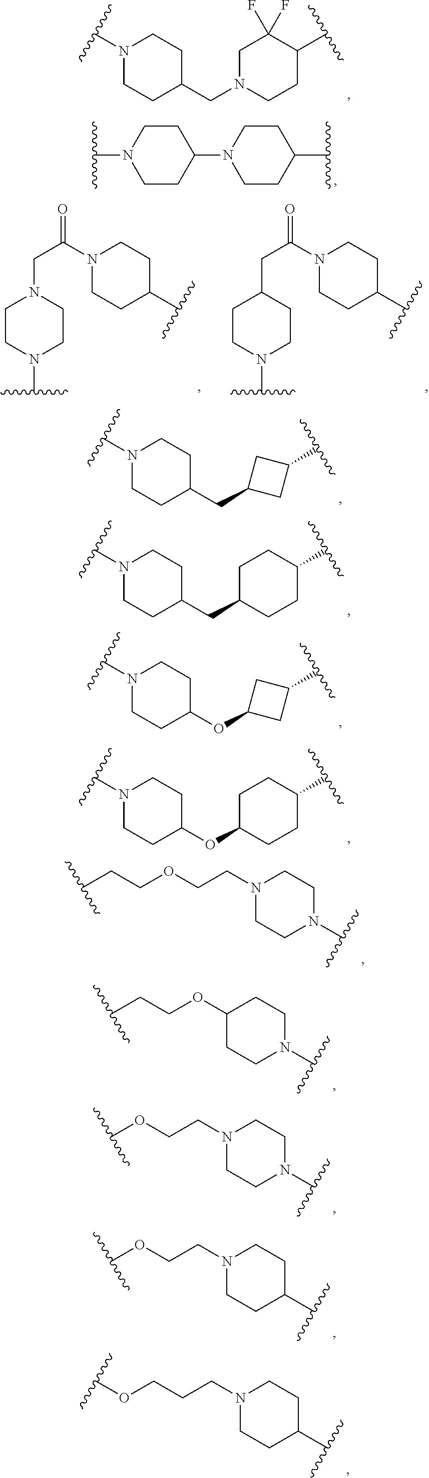

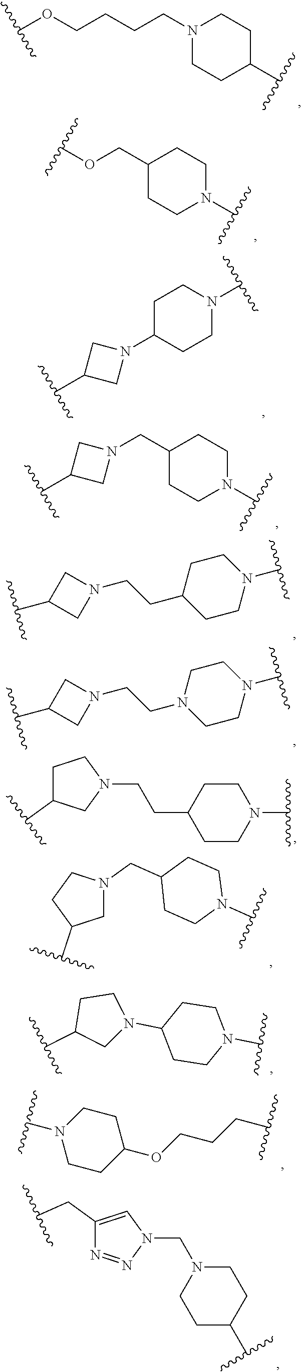

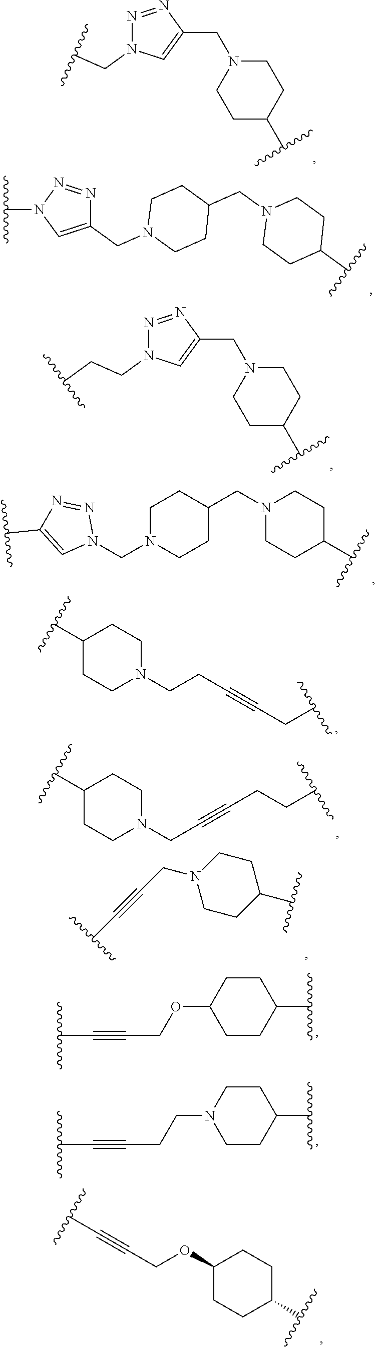

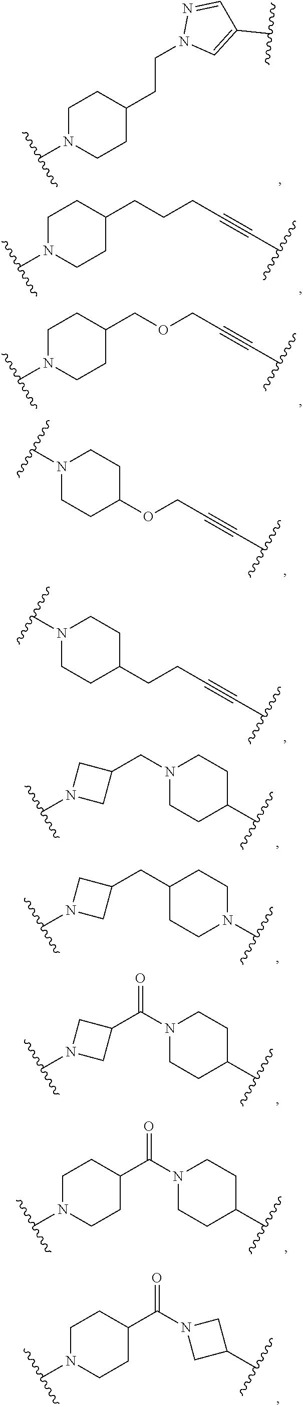

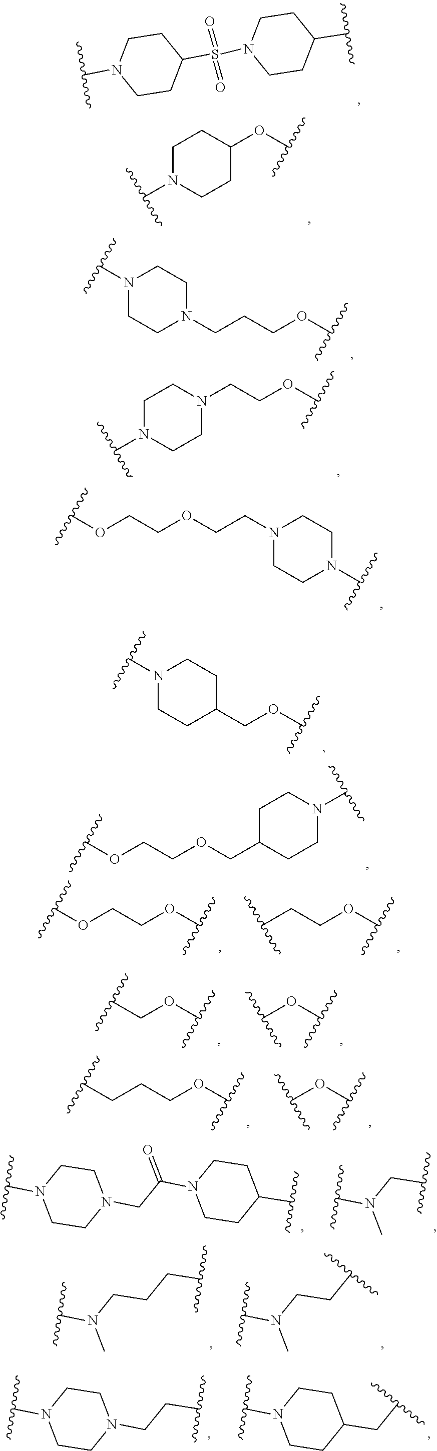

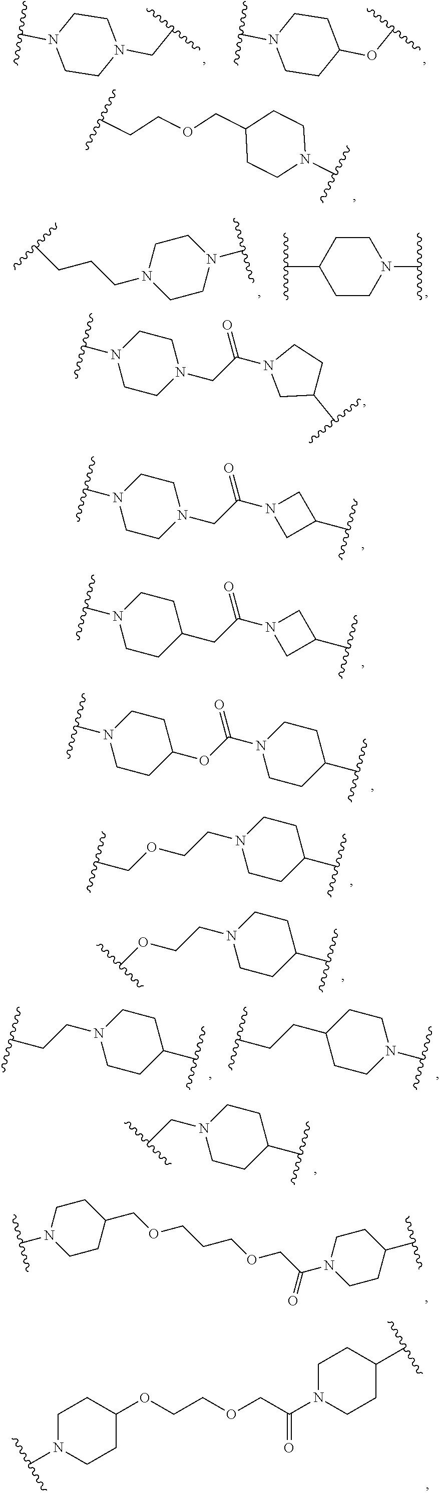

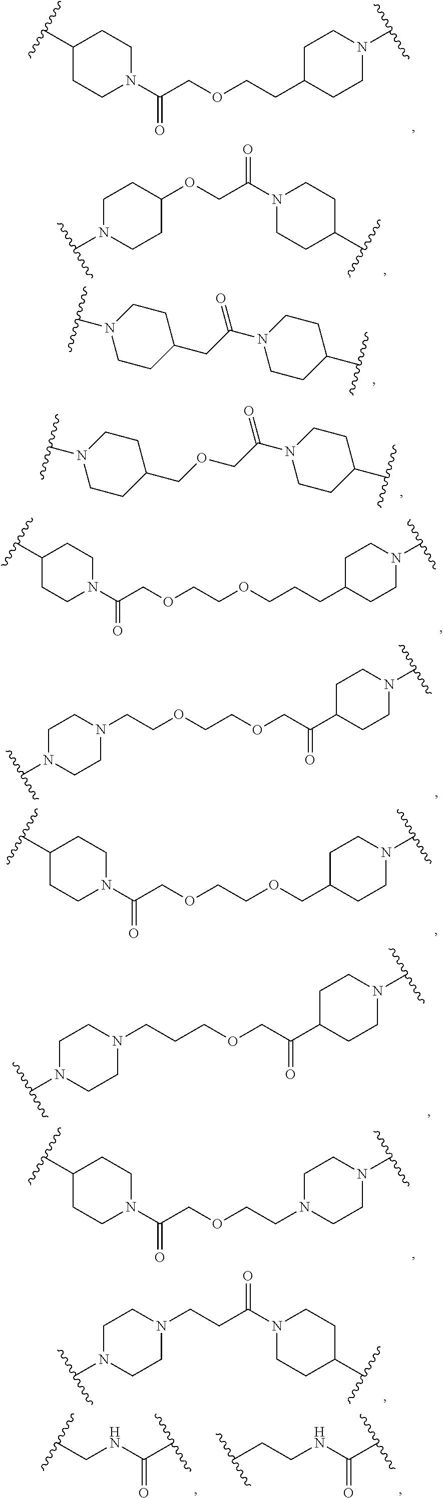

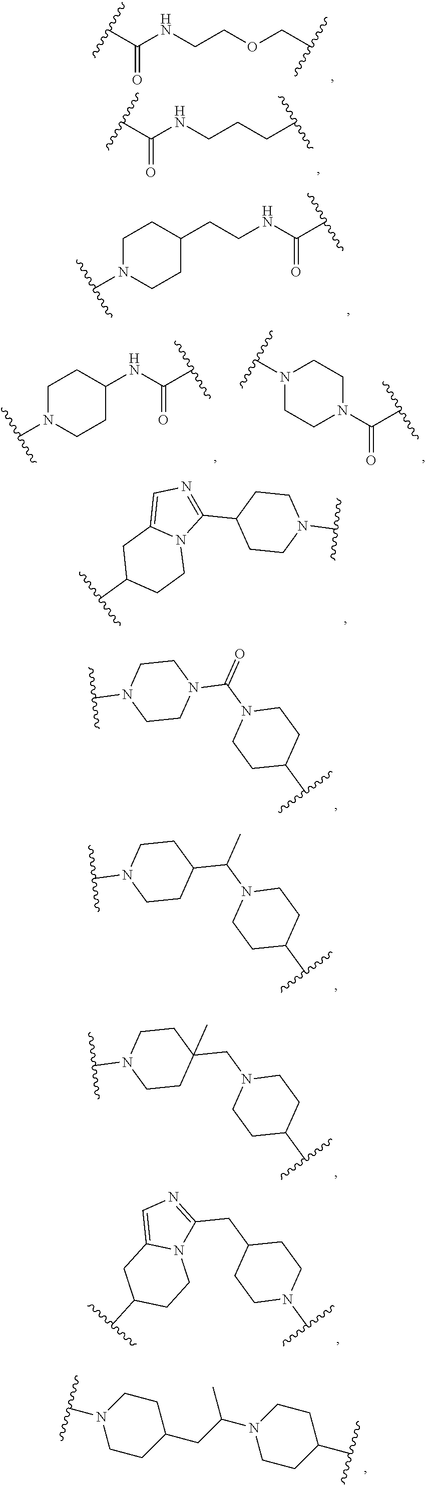

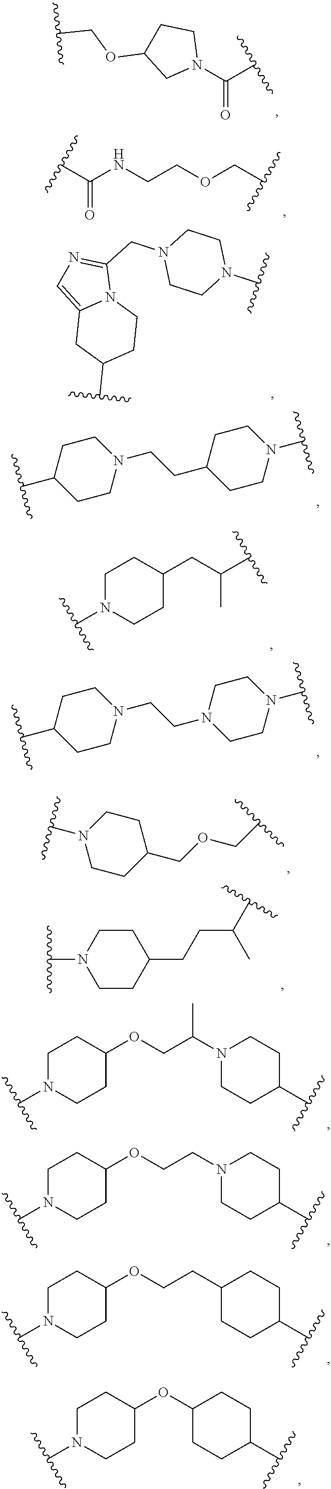

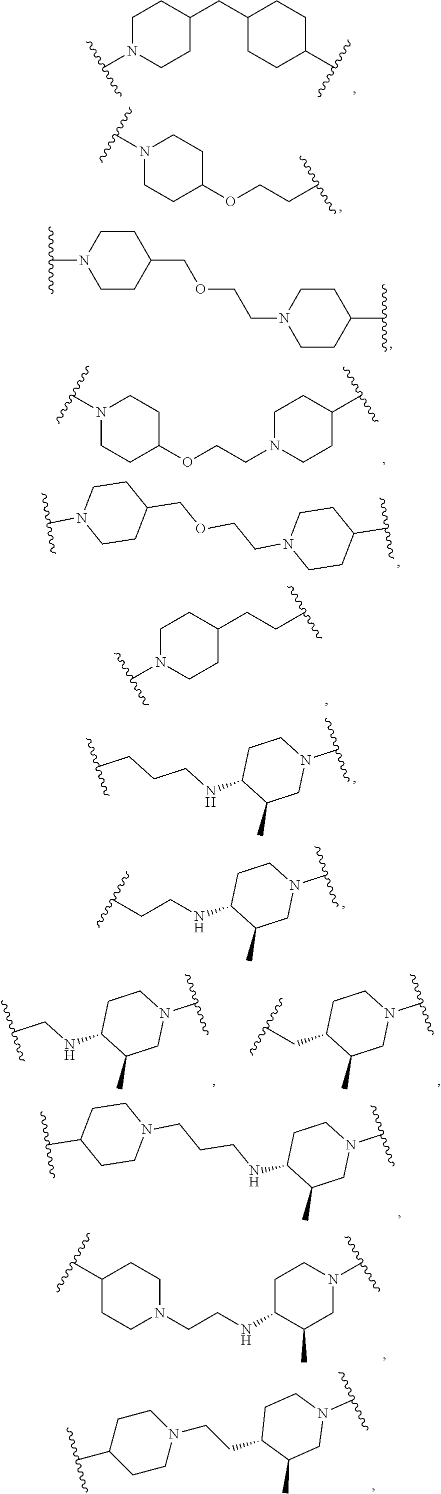

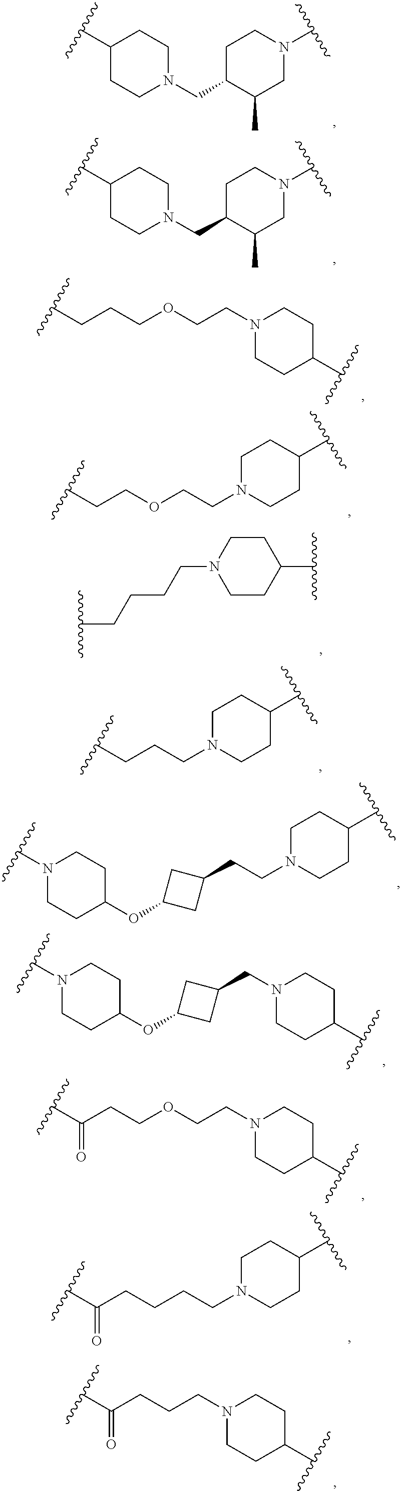

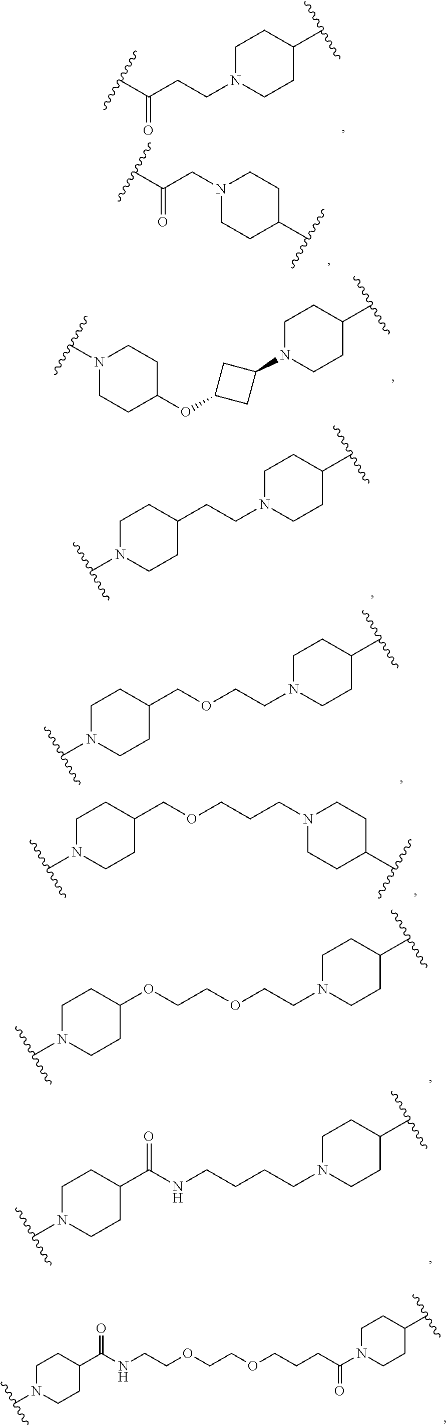

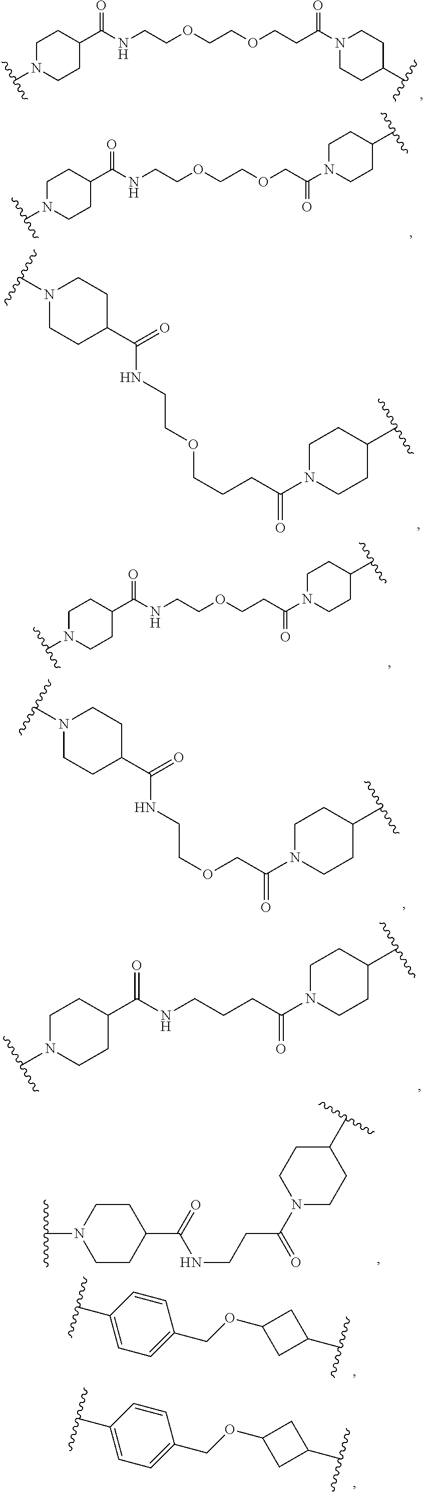

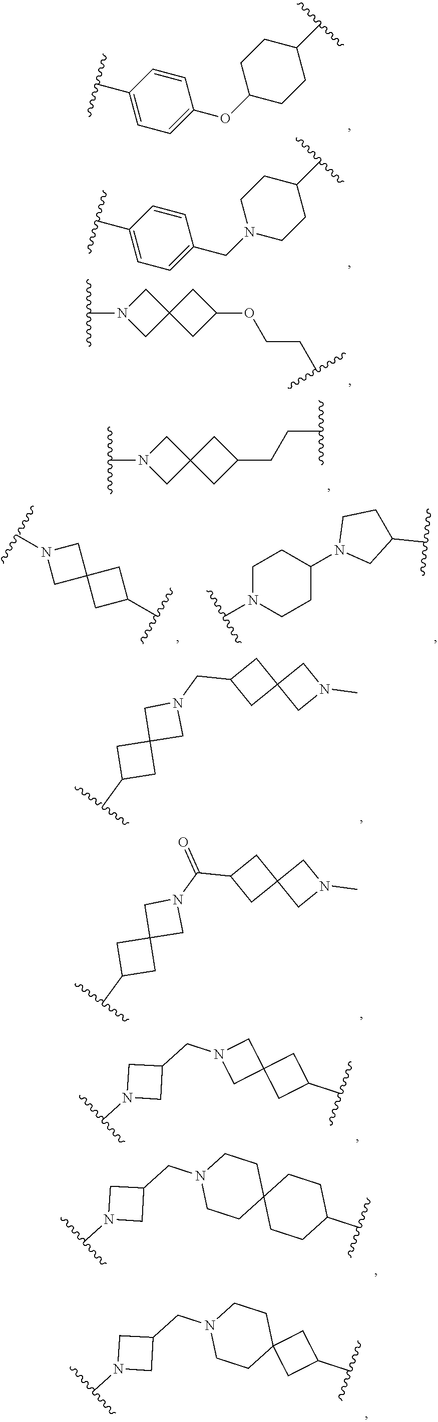

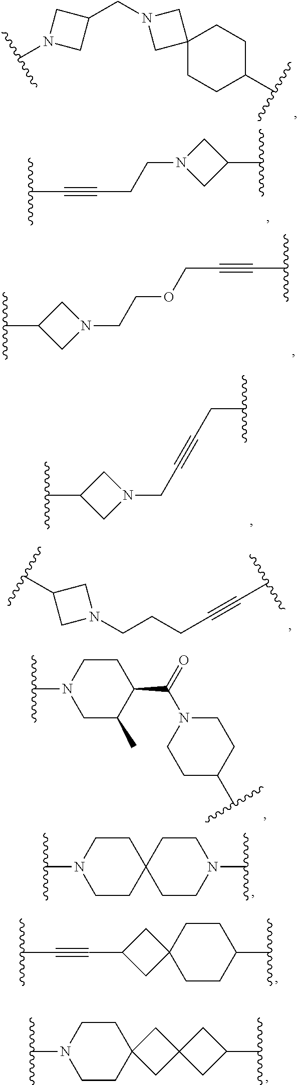

or a pharmaceutically acceptable salt or solvate thereof.

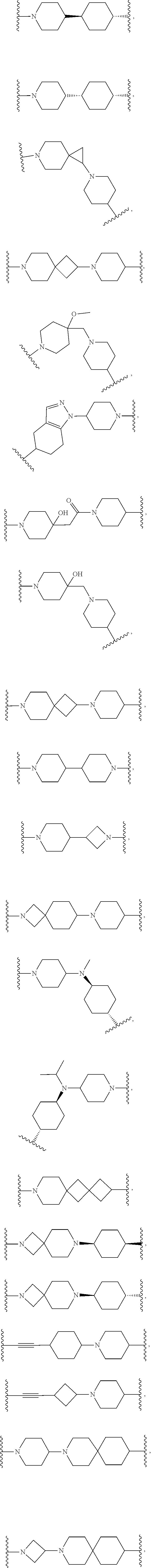

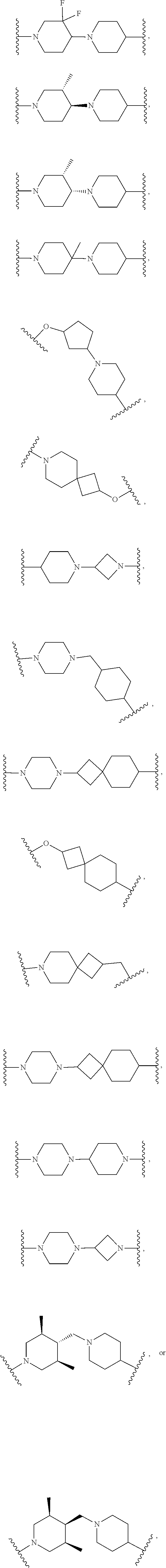

Embodiment 103. The compound of any one of embodiments 1-95, or a pharmaceutically acceptable salt or solvate thereof, wherein L has one of the following structures:

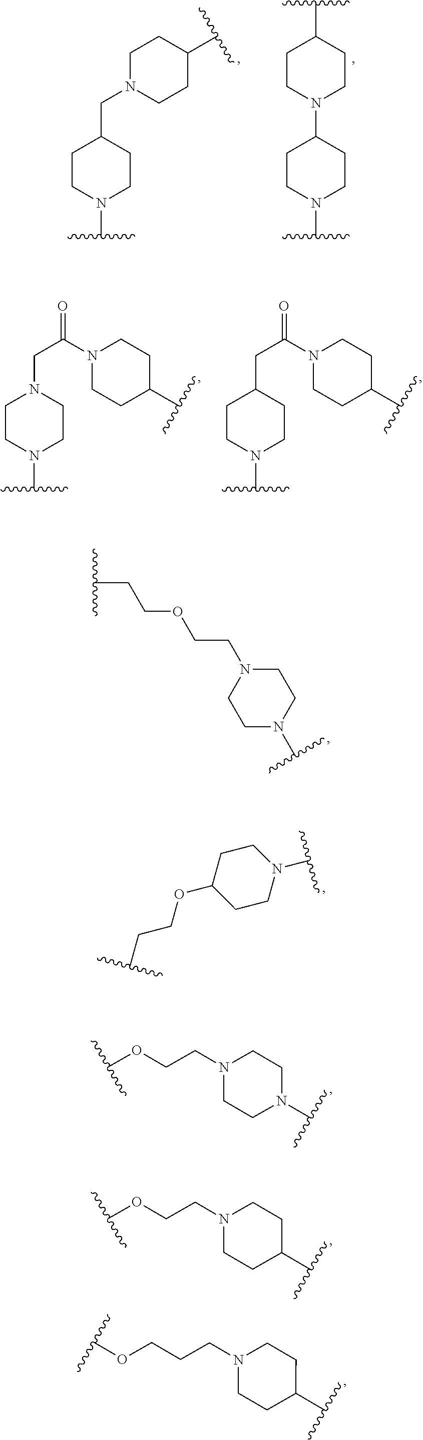

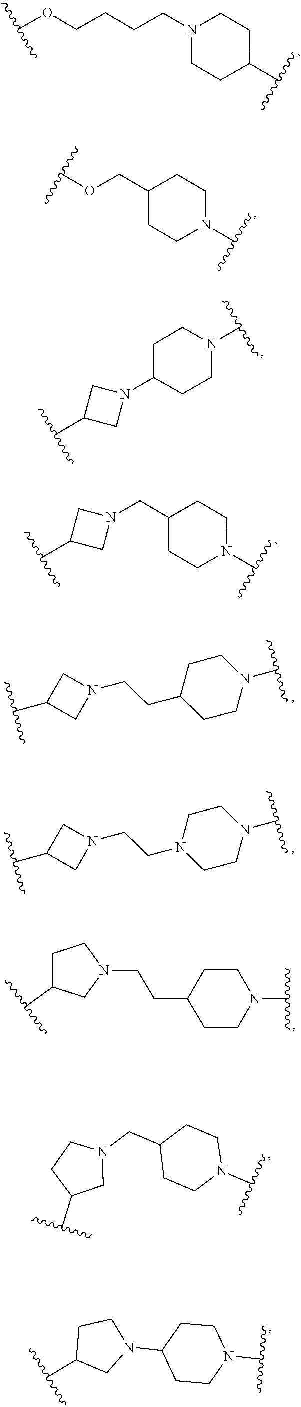

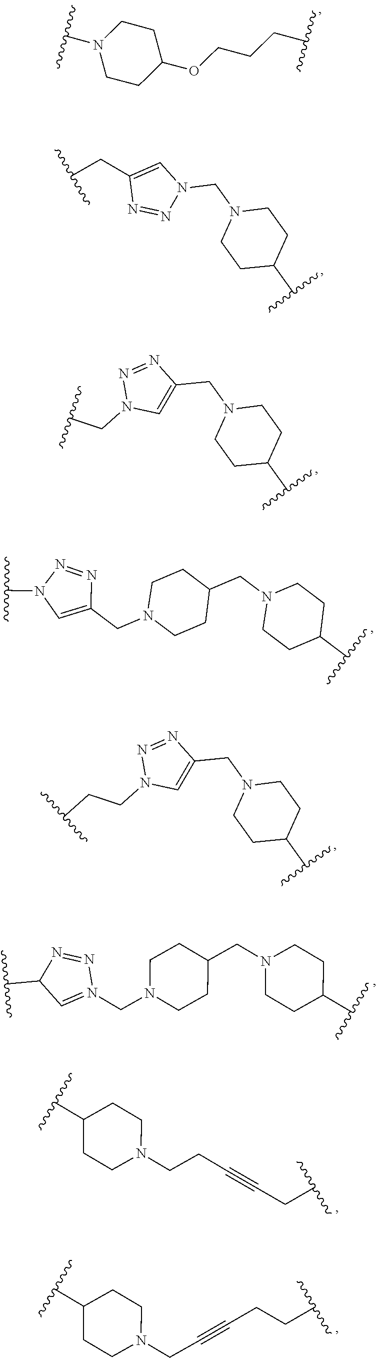

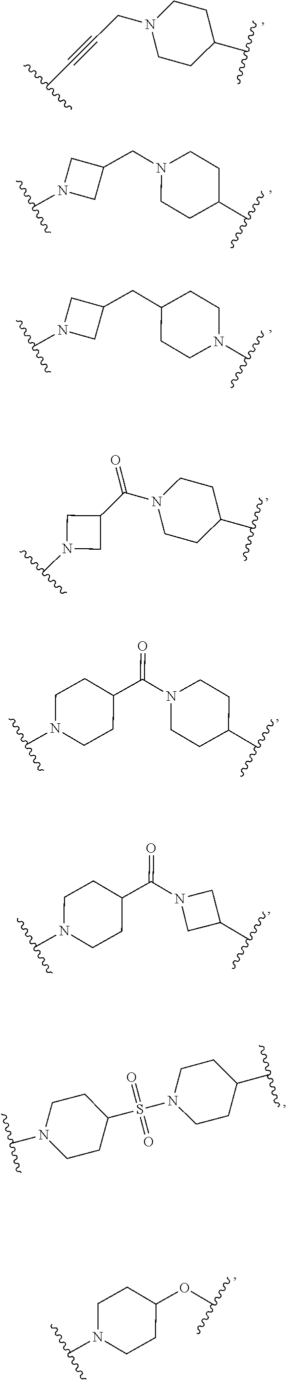

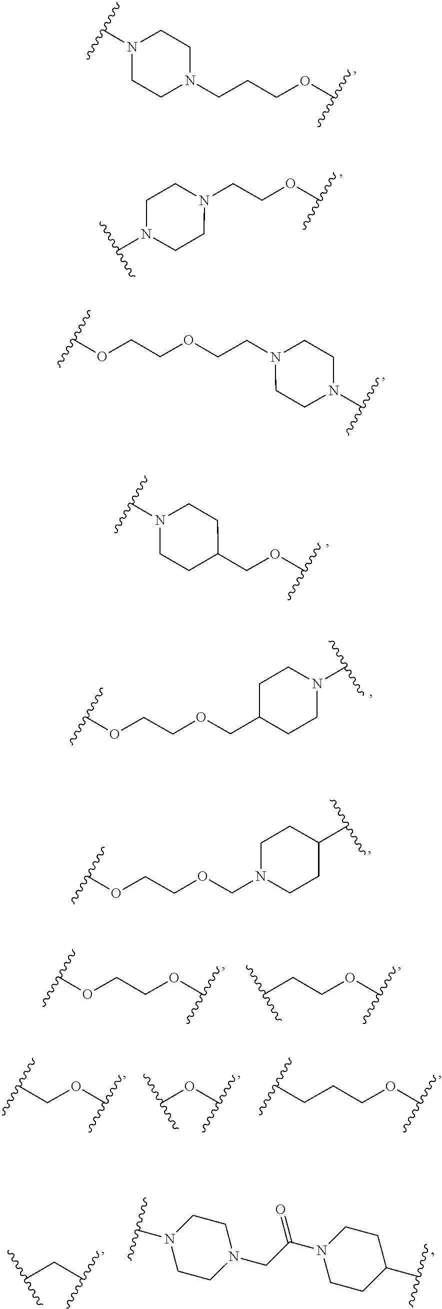

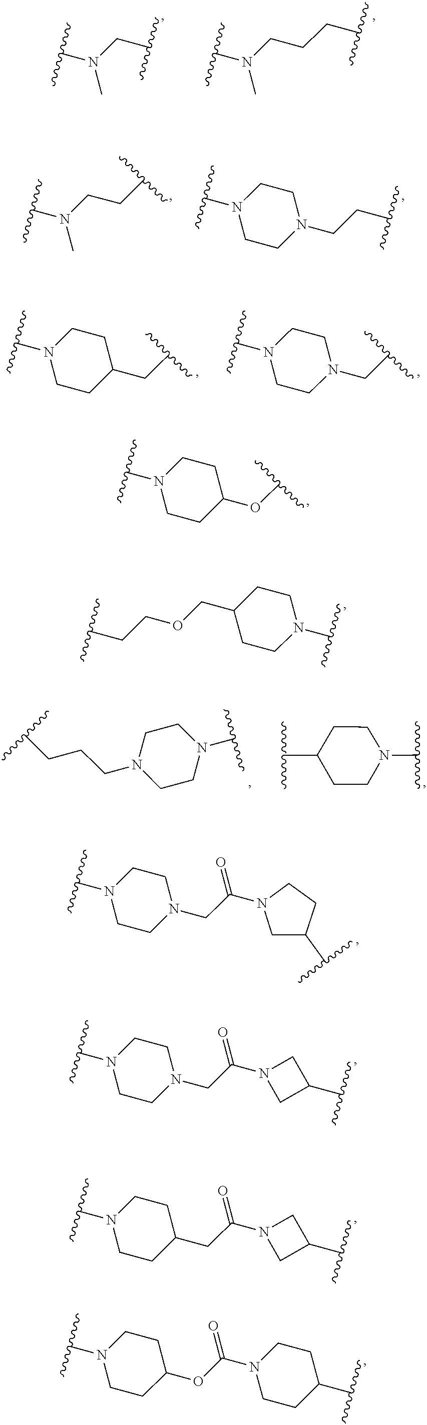

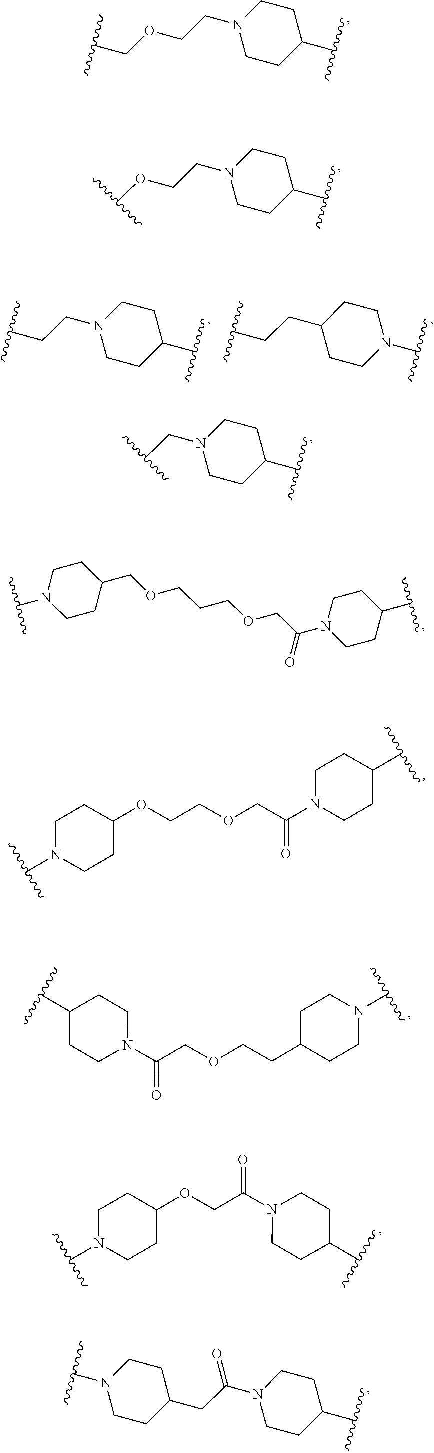

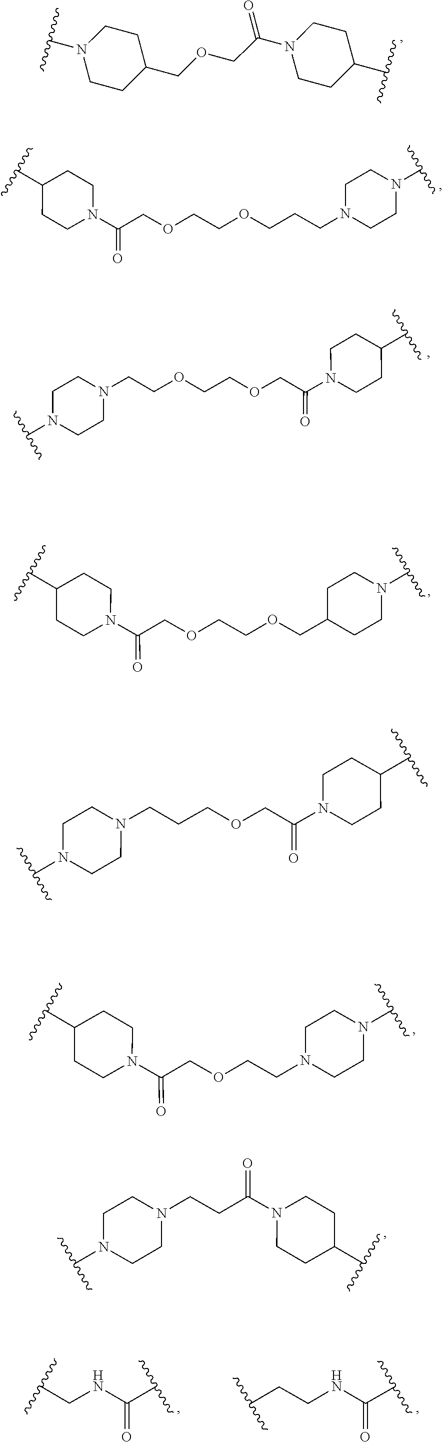

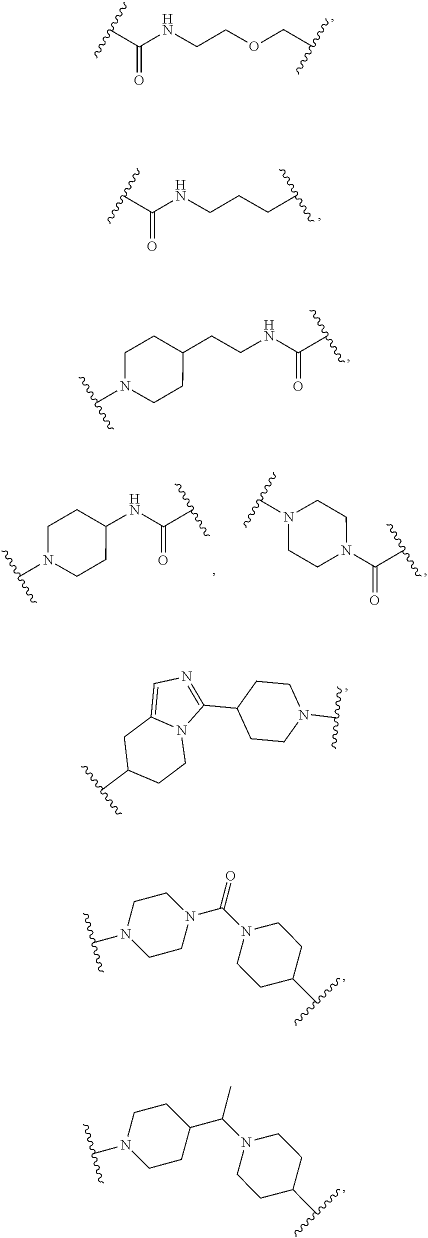

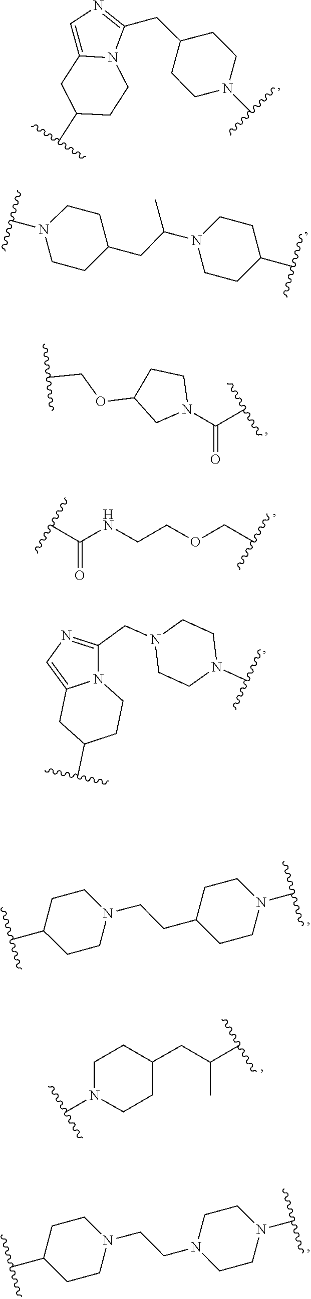

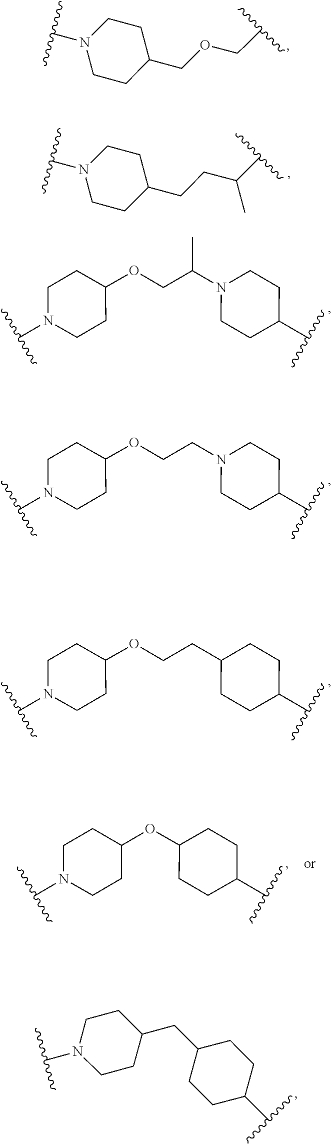

or a pharmaceutically acceptable salt or solvate thereof.

Embodiment 104. A compound, or a pharmaceutically acceptable salt or solvate thereof, that has the following structure:

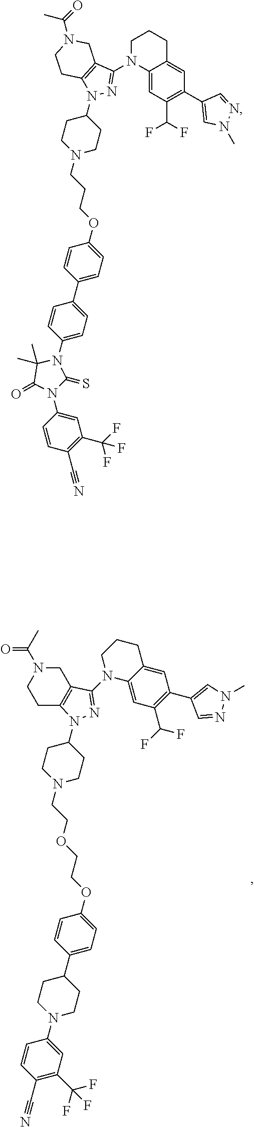 [SMILES: CC(=O)N1CCc2c(c(N3CCCc4cc(-c5cnn(C)c5)c(C(F)F)cc43)nn2C2CCN(CCCOc3ccc(-c4ccc(N5C(=S)N(c6ccc(C#N)c(C(F)(F)F)c6)C(=O)C5(C)C)cc4)cc3)CC2)C1 ; CC(=O)N1CCc2c(c(N3CCCc4cc(-c5cnn(C)c5)c(C(F)F)cc43)nn2C2CCN(CCOCCOc3ccc(C4CCN(c5ccc(C#N)c(C(F)(F)F)c5)CC4)cc3)CC2)C1]

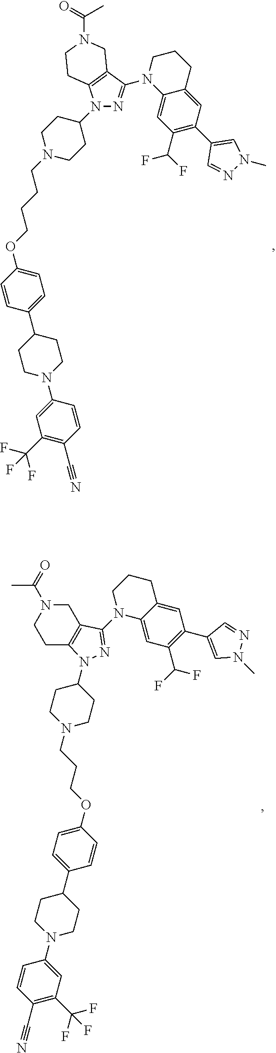

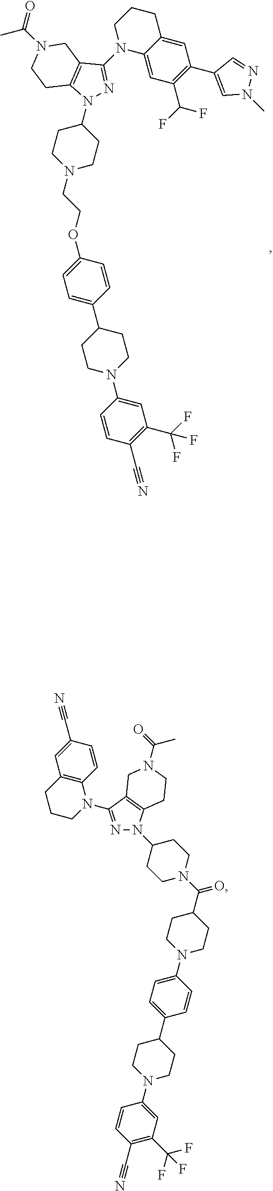 [SMILES: CC(=O)N1CCc2c(c(N3CCCc4cc(-c5cnn(C)c5)c(C(F)F)cc43)nn2C2CCN(CCOc3ccc(C4CCN(c5ccc(C#N)c(C(F)(F)F)c5)CC4)cc3)CC2)C1 ; CC(=O)N1CCc2c(c(N3CCCc4cc(C#N)ccc43)nn2C2CCN(C(=O)C3CCN(c4ccc(C5CCN(c6ccc(C#N)c(C(F)(F)F)c6)CC5)cc4)CC3)CC2)C1]

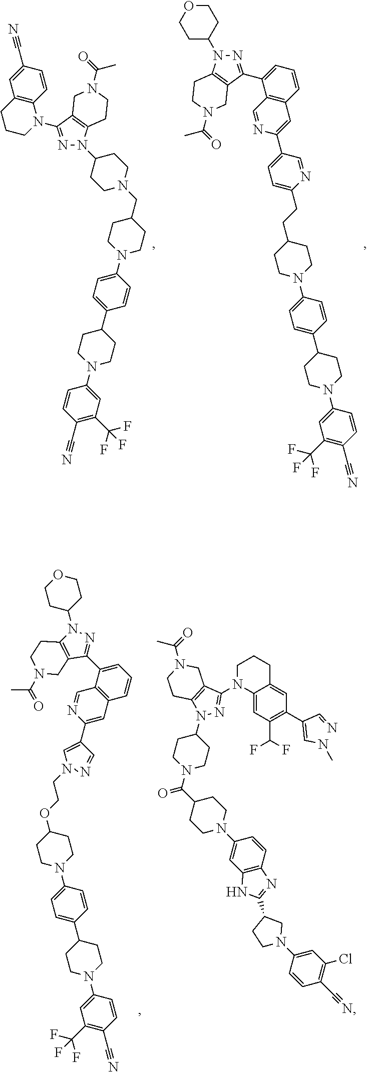 [SMILES: CC(=O)N1CCc2c(c(N3CCCc4cc(C#N)ccc43)nn2C2CCN(CC3CCN(c4ccc(C5CCN(c6ccc(C#N)c(C(F)(F)F)c6)CC5)cc4)CC3)CC2)C1 ; CC(=O)N1CCc2c(c(-c3cccc4cc(-c5ccc(CCC6CCN(c7ccc(C8CCN(c9ccc(C#N)c(C(F)(F)F)c9)CC8)cc7)CC6)nc5)ncc34)nn2C2CCOCC2)C1 ; CC(=O)N1CCc2c(c(-c3cccc4cc(-c5cnn(CCOC6CCN(c7ccc(C8CCN(c9ccc(C#N)c(C(F)(F)F)c9)CC8)cc7)CC6)c5)ncc34)nn2C2CCOCC2)C1 ; CC(=O)N1CCc2c(c(N3CCCc4cc(-c5cnn(C)c5)c(C(F)F)cc43)nn2C2CCN(C(=O)C3CCN(c4ccc5nc([C@H]6CCN(c7ccc(C#N)c(Cl)c7)C6)[nH]c5c4)CC3)CC2)C1]

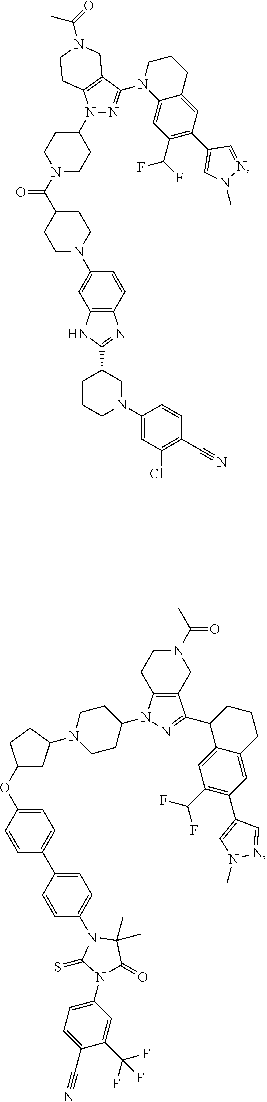 [SMILES: CC(=O)N1CCc2c(c(N3CCCc4cc(-c5cnn(C)c5)c(C(F)F)cc43)nn2C2CCN(C(=O)C3CCN(c4ccc5nc([C@H]6CCCN(c7ccc(C#N)c(Cl)c7)C6)[nH]c5c4)CC3)CC2)C1 ; CC(=O)N1CCc2c(c(C3CCCc4cc(-c5cnn(C)c5)c(C(F)F)cc43)nn2C2CCN(C3CCC(Oc4ccc(-c5ccc(N6C(=S)N(c7ccc(C#N)c(C(F)(F)F)c7)C(=O)C6(C)C)cc5)cc4)C3)CC2)C1]

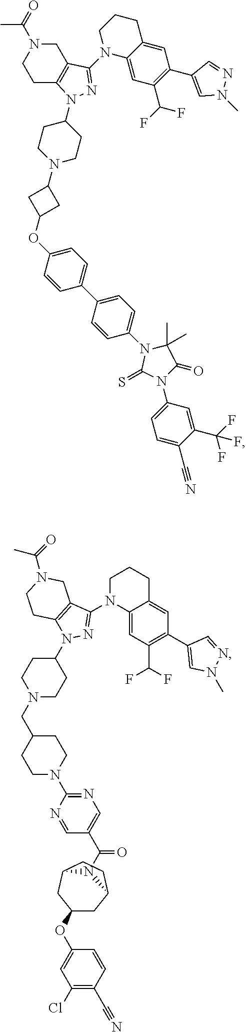 [SMILES: CC(=O)N1CCc2c(c(N3CCCc4cc(-c5cnn(C)c5)c(C(F)F)cc43)nn2C2CCN(C3CC(Oc4ccc(-c5ccc(N6C(=S)N(c7ccc(C#N)c(C(F)(F)F)c7)C(=O)C6(C)C)cc5)cc4)C3)CC2)C1 ; CC(=O)N1CCc2c(c(N3CCCc4cc(-c5cnn(C)c5)c(C(F)F)cc43)nn2C2CCN(CC3CCN(c4ncc(C(=O)N5[C@@H]6CC[C@H]5C[C@@H](Oc5ccc(C#N)c(Cl)c5)C6)cn4)CC3)CC2)C1]

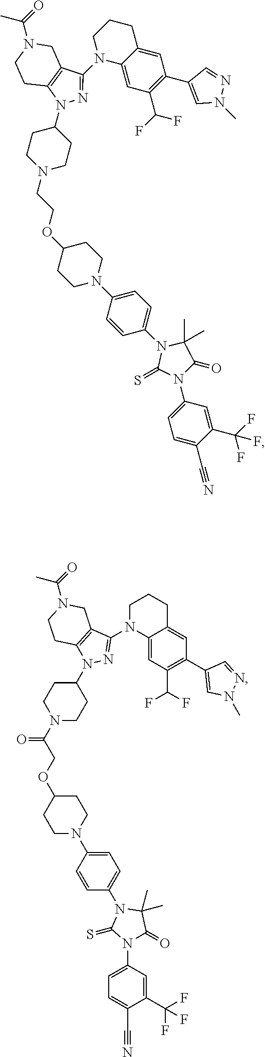 [SMILES: CC(=O)N1CCc2c(c(N3CCCc4cc(-c5cnn(C)c5)c(C(F)F)cc43)nn2C2CCN(CCOC3CCN(c4ccc(N5C(=S)N(c6ccc(C#N)c(C(F)(F)F)c6)C(=O)C5(C)C)cc4)CC3)CC2)C1 ; CC(=O)N1CCc2c(c(N3CCCc4cc(-c5cnn(C)c5)c(C(F)F)cc43)nn2C2CCN(C(=O)COC3CCN(c4ccc(N5C(=S)N(c6ccc(C#N)c(C(F)(F)F)c6)C(=O)C5(C)C)cc4)CC3)CC2)C1]

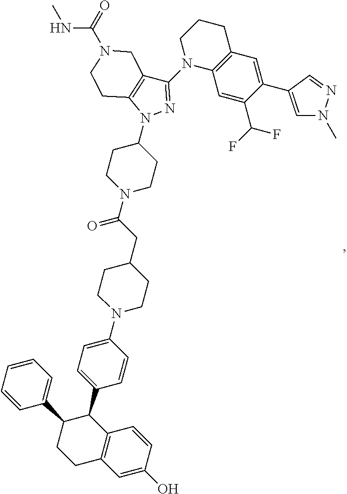 [SMILES: CNC(=O)N1CCc2c(c(N3CCCc4cc(-c5cnn(C)c5)c(C(F)F)cc43)nn2C2CCN(C(=O)CC3CCN(c4ccc([C@H]5c6ccc(O)cc6CC[C@H]5c5ccccc5)cc4)CC3)CC2)C1]

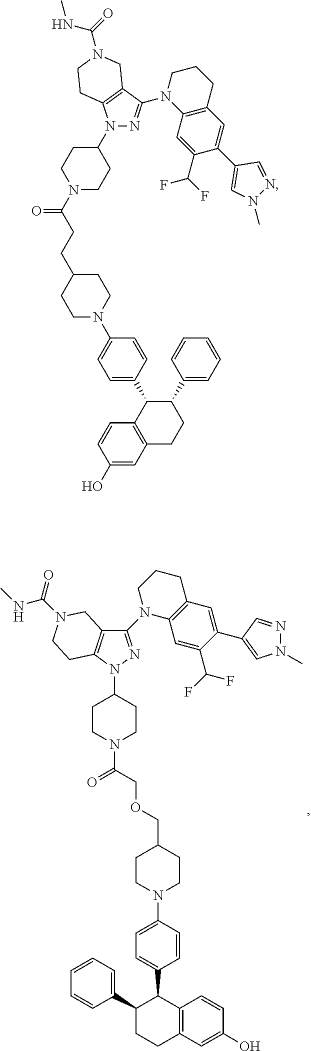

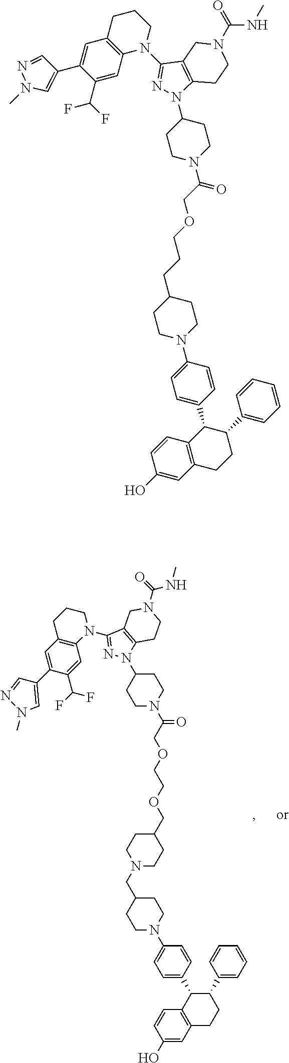 [SMILES: CNC(=O)N1CCc2c(c(N3CCCc4cc(-c5cnn(C)c5)c(C(F)F)cc43)nn2C2CCN(C(=O)COCCCC3CCN(c4ccc([C@H]5c6ccc(O)cc6CC[C@H]5c5ccccc5)cc4)CC3)CC2)C1 ; CNC(=O)N1CCc2c(c(N3CCCc4cc(-c5cnn(C)c5)c(C(F)F)cc43)nn2C2CCN(C(=O)COCCOCC3CCN(CC4CCN(c5ccc([C@H]6c7ccc(O)cc7CC[C@H]6c6ccccc6)cc5)CC4)CC3)CC2)C1]

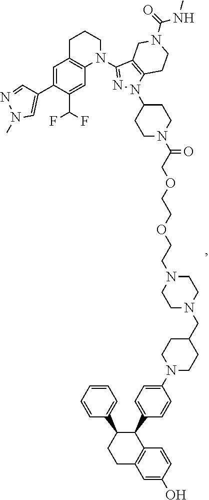 [SMILES: CNC(=O)N1CCc2c(c(N3CCCc4cc(-c5cnn(C)c5)c(C(F)F)cc43)nn2C2CCN(C(=O)COCCOCCN3CCN(CC4CCN(c5ccc([C@H]6c7ccc(O)cc7CC[C@H]6c6ccccc6)cc5)CC4)CC3)CC2)C1]

or a pharmaceutically acceptable salt or solvate thereof.

Embodiment 105. A stable ternary complex comprising:
a. one or more disease-dependent proteins (DDPs);
b. a disease protein (DP); and
c. heterobifunctional conditional inhibitor compound of Formula (I):

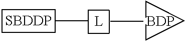

wherein:
SBDDP is a silent binder of a disease-dependent protein (DDP);
L is an optional linker; and
BDP is a binder of a disease protein (DP);
wherein DDP and DP are present in a cell of interest (COI) and the relative abundance of the DP in the COI is greater than the relative abundance of the DDP in the COI.

Embodiment 106. The stable ternary complex of embodiment 105, wherein DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3.

Embodiment 107. The stable ternary complex of embodiment 106, wherein DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

Embodiment 108. The compound of embodiment 106, wherein DDP is a human bromodomain-containing protein (BRD).

Embodiment 109. The compound of embodiment 106, wherein DDP is a human bromodomain-containing protein that is a Group Ia BRD, Group Ib BRD, Group II BRD, Group IIIa BRD, Group IIIb BRD, Group IIII BRD, Group IV BRD, Group VI BRD, Group VI BRD, Group VIII BRD, or group IX BRD;
wherein:
the Group Ia BRD is selected from PCAF, GCN5L2, p300/CBP, TAF1 and TAF1L;
the Group Ib BRD is selected from BRPF1A/B (BR140), BRPF2 (BRD1) BRPF3 and BRD8 (SMAP);
the Group II BRD is selected from histone methyltransferases ASH1L and MLL;
the Group IIIa BRD is selected from chromatin remodeling factors SMARCA2 (BRM), SMARCA4 (BRG1), BRD7, BRD9 and PBRM1 (polybromo);
the Group IIIb BRD is selected from ISWI family chromatin remodeling factors BAZ1A (ACF1), BAZ1B (WSTF, William-Beuren syndrome transcription factor), BAZ2A, BAZ2B, BPTF and CECR2;
the Group IV BRD is selected from ATPase family AAA domain-containing proteins ATAD2 and ATAD2B;
the Group VI BRD is selected from Tripartite-motif-containing (TRIM) family proteins TRIM24 (TIF1α), TRIM28 (TIF1β, KAP1), TRIM33 (TIF1γ) and TRIM66 (TIF1δ);
the Group VIII BRD is selected from speckled protein (SP) family proteins SP100, SP110, SP140 and SP140L;
the Group VIII BRD is selected from transcriptional corepressors ZMYND8 and ZMYDN11; and
the Group IX is selected from WD-repeat proteins BRWD1 (WDR9), BRWD3 and PHIP (WDR11).

Embodiment 110. The compound of embodiment 108, wherein DDP is a BRD selected from PCAF, GCN5L2, p300/CBP, TAF1, TAF1L, BRPF1A/B (BR140), BRPF2 (BRD1) BRPF3, BRD8 (SMAP), chromatin remodeling factors SMARCA2 (BRM), SMARCA4 (BRG1), BRD7, BRD9, PBRM1 (polybromo), BAZ1A (ACF1), BAZ1B (WSTF, William-Beuren syndrome transcription factor), BAZ2A, BAZ2B, BPTF, CECR2, ATAD2, ATAD2B, Tripartite-motif-containing (TRIM) family proteins TRIM24 (TIF1α), TRIM28 (TIF1β, KAP1), TRIM33 (TIF1γ), TRIM66 (TIF1δ), WD-repeat proteins BRWD1 (WDR9), BRWD3 and PHIP (WDR11).

Embodiment 111. A stable ternary complex comprising:
a. CBP/p300;
b. a disease protein (DP); and
c. heterobifunctional conditional inhibitor compound of Formula (II):

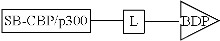

wherein:
SB-CBP/p300 is a silent binder of CBP/p300;
L is an optional linker; and
BDP is a binder of a disease protein (DP);
wherein CBP/p300 and DP are present in a cell of interest (COI) and the relative abundance of the DP in the COI is greater than the relative abundance of CBP/p300 in the COI.

Embodiment 112. The stable ternary complex of any one of embodiments 105-111, wherein DP is a nuclear hormone receptor protein, G-protein coupled receptor, epidermal growth factor receptor, RAS protein, or transcription factor.

Embodiment 113. The stable ternary complex of any one of embodiments 105-111, wherein DP is androgen receptor (AR), estrogen receptor (ER), progesterone receptor (PR), glucocorticoid receptor (GR), mineralocorticoid receptor (MR), angiotensin receptor, apelin receptor, bombesin receptor, bradykinin receptor, calcitonin receptor, chemokine receptor, cholecytokinin receptor, corticotropic-releasing factor receptor, galanin receptor, ghrelin receptor, glucagon receptor, glycoprotein hormone receptor, gonadotropin-releasing hormone receptor, kisspeptin receptor, melanocortin receptor, motilin receptor, neuromedin U receptor, neuropeptide FF/AF receptor, neuropeptide S receptor, neuropeptide W/B receptor, neuropeptide Y receptor, opioid receptor, orexin receptor, parathyroid hormone receptor, prokineticin receptor, prolactin-releasing peptide receptor, QRFP receptor, relaxin family peptide receptor, somatostatin receptor, tachykinin receptor, thyrotropin-releasing hormone receptor, urotensin receptor, vasopressin, oxytocin receptor, vasoactive intestinal peptide receptor, PACAP receptor, HER1, HER2, HER3, HER4, KRAS, KRAS-G12C, HRAS, NRAS, or BCL6.

Embodiment 114. The compound of any one of embodiments 105-111, wherein the DP is androgen receptor (AR), estrogen receptor (ER), progesterone receptor (PR), glucocorticoid receptor (GR), mineralocorticoid receptor (MR), retinoic acid receptor-related orphan nuclear receptor γ (RORγ), apelin receptor, bombesin receptor, bradykinin receptor, chemokine receptor, cholecytokinin receptor, chymase, gonadotropin-releasing hormone receptor, indoleamine 2,3-dioxygenase 1 (IDO1), kisspeptin receptor, neuromedin U receptor, neuropeptide FF/AF receptor, neuropeptide S receptor, neuropeptide W/B receptor, neuropeptide Y receptor, somatostatin receptor, tachykinin receptor, HER1, HER2, HER3, HER4, KRAS, KRAS-G12C, HRAS, NRAS, or BCL6.

Embodiment 115. The stable ternary complex of any one of embodiments 105-111, wherein DP is androgen receptor (AR) or estrogen receptor (ER).

Embodiment 116. The stable ternary complex of any one of embodiments 105-111, wherein DP is androgen receptor (AR).

Embodiment 117. The stable ternary complex of any one of embodiments 105-111, wherein DP is androgen receptor (AR) or DP is estrogen receptor (ER) and the heterobifunctional conditional inhibitor compound is the compound of any one of embodiments 64-88 or 104.

Embodiment 118. A stable ternary complex comprising:
a. CBP/p300;
b. Androgen receptor (AR) or Estrogen receptor (ER); and
c. heterobifunctional conditional inhibitor compound of any one of embodiments 64-88 or 104;
wherein CBP/p300 and DP are present in a cell of interest (COI) and the relative abundance of the DP in the COI is greater than the relative abundance of CBP/p300 in the COI.

Embodiment 119. A method of selectively inhibiting the activity of a disease-dependent protein (DDP) in a cell of interest (COI) of a mammal comprising administering a heterobifunctional compound of any one of embodiments 1-104, or a pharmaceutically acceptable salt or solvate thereof, wherein the heterobifunctional compound inhibits the activity of the DDP in the COI but does not inhibit the activity of the DDP in cells expressing the DDP and not expressing the DP.

Embodiment 120. The method of embodiment 119, wherein the DP is overexpressed, overactive or both overexpressed and overactive in the COI.

Embodiment 121. The method of embodiment 119, wherein DP is a nuclear hormone receptor protein, G-protein coupled receptor, epidermal growth factor receptor, RAS protein, or transcription factor.

Embodiment 122. The method of embodiment 119 or embodiment 120, wherein DP is androgen receptor (AR), estrogen receptor (ER), progesterone receptor (PR), glucocorticoid receptor (GR), mineralocorticoid receptor (MR), angiotensin receptor, apelin receptor, bombesin receptor, bradykinin receptor, calcitonin receptor, chemokine receptor, cholecytokinin receptor, corticotropic-releasing factor receptor, galanin receptor, ghrelin receptor, glucagon receptor, glycoprotein hormone receptor, gonadotropin-releasing hormone receptor, kisspeptin receptor, melanocortin receptor, motilin receptor, neuromedin U receptor, neuropeptide FF/AF receptor, neuropeptide S receptor, neuropeptide W/B receptor, neuropeptide Y receptor, opioid receptor, orexin receptor, parathyroid hormone receptor, prokineticin receptor, prolactin-releasing peptide receptor, QRFP receptor, relaxin family peptide receptor, somatostatin receptor, tachykinin receptor, thyrotropin-releasing hormone receptor, urotensin receptor, vasopressin, oxytocin receptor, vasoactive intestinal peptide receptor, PACAP receptor, HER1, HER2, HER3, HER4, KRAS, KRAS-G12C, HRAS, NRAS, or BCL6.

Embodiment 123. The method of embodiment 119 or embodiment 120, wherein the DP is androgen receptor (AR), estrogen receptor (ER), progesterone receptor (PR), glucocorticoid receptor (GR), mineralocorticoid receptor (MR), retinoic acid receptor-related orphan nuclear receptor γ (RORγ), apelin receptor, bombesin receptor, bradykinin receptor, chemokine receptor, cholecytokinin receptor, chymase, gonadotropin-releasing hormone receptor, indoleamine 2,3-dioxygenase 1 (IDO1), kisspeptin receptor, neuromedin U receptor, neuropeptide FF/AF receptor, neuropeptide S receptor, neuropeptide W/B receptor, neuropeptide Y receptor, somatostatin receptor, tachykinin receptor, HER1, HER2, HER3, HER4, KRAS, KRAS-G12C, HRAS, NRAS, or BCL6.

Embodiment 124. The method of embodiment 119 or embodiment 120, wherein DP is AR or ER.

Embodiment 125. The method of embodiment 119 or embodiment 120, wherein DP is androgen receptor (AR).

Embodiment 126. The method of any one of embodiments 118-125, wherein DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC 1), SRC2, or SRC3.

Embodiment 127. The method of any one of embodiments 118-125, wherein DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

Embodiment 128. The method of any one of embodiments 118-125, wherein DDP is a BRD selected from PCAF, GCN5L2, p300/CBP, TAF1, TAF1L, BRPF1A/B (BR140), BRPF2 (BRD1) BRPF3, BRD8 (SMAP), chromatin remodeling factors SMARCA2 (BRM), SMARCA4 (BRG1), BRD7, BRD9, PBRM1 (polybromo), BAZ1A (ACF1), BAZ1B (WSTF, William-Beuren syndrome transcription factor), BAZ2A, BAZ2B, BPTF, CECR2, ATAD2, ATAD2B, Tripartite-motif-containing (TRIM) family proteins TRIM24 (TIF1α), TRIM28 (TIF1β, KAP1), TRIM33 (TIF1γ), TRIM66 (TIF1δ), WD-repeat proteins BRWD1 (WDR9), BRWD3 and PHIP (WDR11).

Embodiment 129. The method of any one of embodiments 118-125, wherein the DDP is CREB-binding protein (CBP)/p300.

Embodiment 130. A method of treating cancer in a mammal comprising administering a therapeutically effective amount of to the mammal a heterobifunctional compound of any one of embodiments 1-104, or a pharmaceutically acceptable salt or solvate thereof.

Embodiment 131. The method of embodiment 130, wherein the cancer is a hormone dependent cancer.

Embodiment 132. The method of embodiment 130, wherein the cancer is adenomas and neoplasms of the prostate, tumor cells expressing the androgen receptor, tumor cells expressing the estrogen receptor, prostate cancer, breast cancer, endometrial cancer, or uterine cancer.

Embodiment 133. A pharmaceutical composition comprising a compound of any one of embodiments 1-104, or a pharmaceutically acceptable salt or solvate thereof, and at least one pharmaceutically acceptable excipient.

In some embodiments, the compound is a compound of Table 1, or a pharmaceutically acceptable salt or solvate thereof:

TABLE 1

| Compound | Structure |
|---|---|
| 1 | 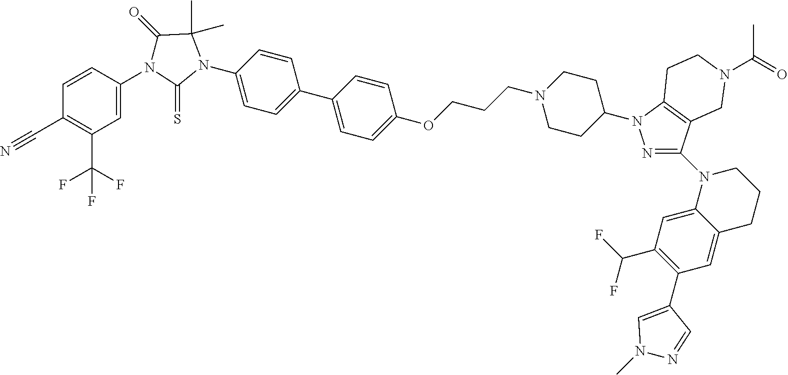 [SMILES: CC(=O)N1CCc2c(c(N3CCCc4cc(-c5cnn(C)c5)c(C(F)F)cc43)nn2C2CCN(CCCOc3ccc(-c4ccc(N5C(=S)N(c6ccc(C#N)c(C(F)(F)F)c6)C(=O)C5(C)C)cc4)cc3)CC2)C1] |
| 2 | 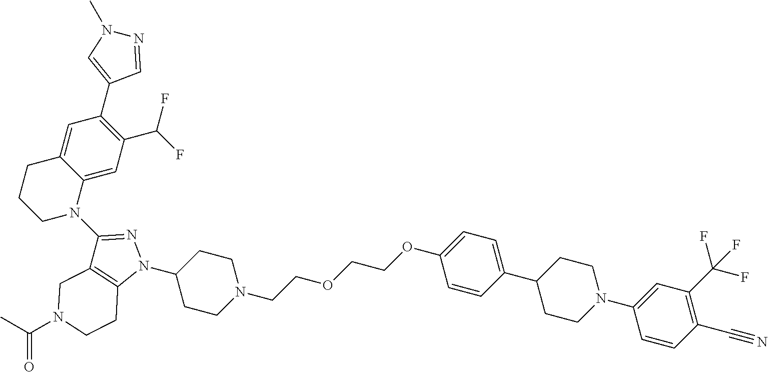 [SMILES: CC(=O)N1CCc2c(c(N3CCCc4cc(-c5cnn(C)c5)c(C(F)F)cc43)nn2C2CCN(CCOCCOc3ccc(C4CCN(c5ccc(C#N)c(C(F)(F)F)c5)CC4)cc3)CC2)C1] |
| 3 | 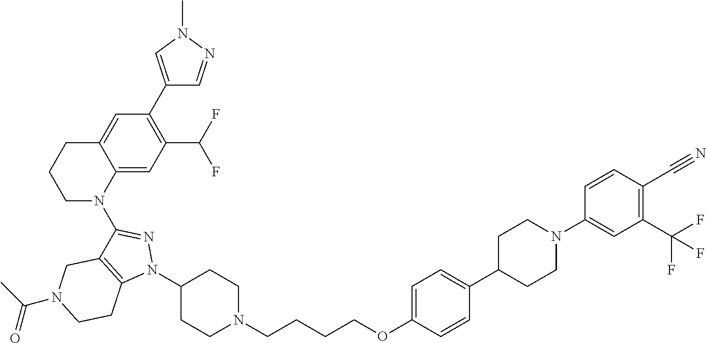 [SMILES: CC(=O)N1CCc2c(c(N3CCCc4cc(-c5cnn(C)c5)c(C(F)F)cc43)nn2C2CCN(CCCCOc3ccc(C4CCN(c5ccc(C#N)c(C(F)(F)F)c5)CC4)cc3)CC2)C1] |
| 4 | 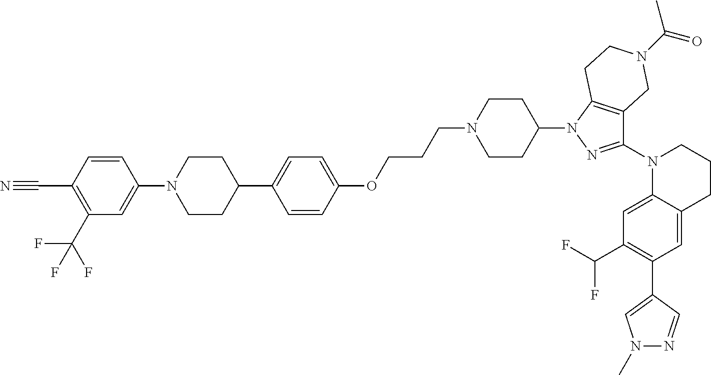 [SMILES: CC(=O)N1CCc2c(c(N3CCCc4cc(-c5cnn(C)c5)c(C(F)F)cc43)nn2C2CCN(CCCOc3ccc(C4CCN(c5ccc(C#N)c(C(F)(F)F)c5)CC4)cc3)CC2)C1] |
| 5 | 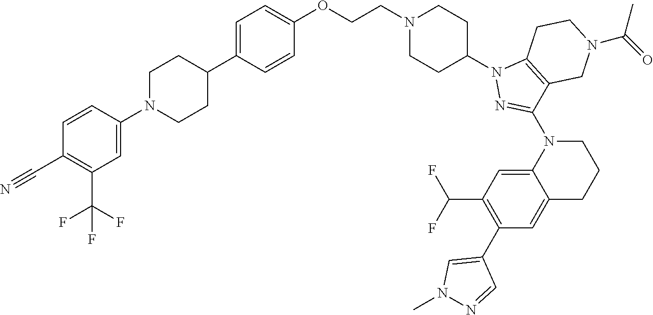 [SMILES: CC(=O)N1CCc2c(c(N3CCCc4cc(-c5cnn(C)c5)c(C(F)F)cc43)nn2C2CCN(CCOc3ccc(C4CCN(c5ccc(C#N)c(C(F)(F)F)c5)CC4)cc3)CC2)C1] |
| 6 | 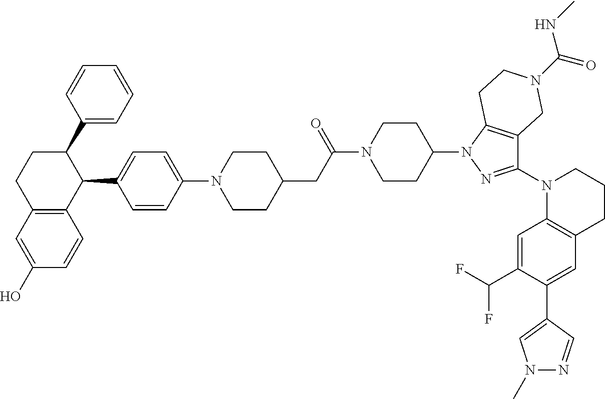 [SMILES: CNC(=O)N1CCc2c(c(N3CCCc4cc(-c5cnn(C)c5)c(C(F)F)cc43)nn2C2CCN(C(=O)CC3CCN(c4ccc([C@H]5c6ccc(O)cc6CC[C@H]5c5ccccc5)cc4)CC3)CC2)C1] |
| 7 | 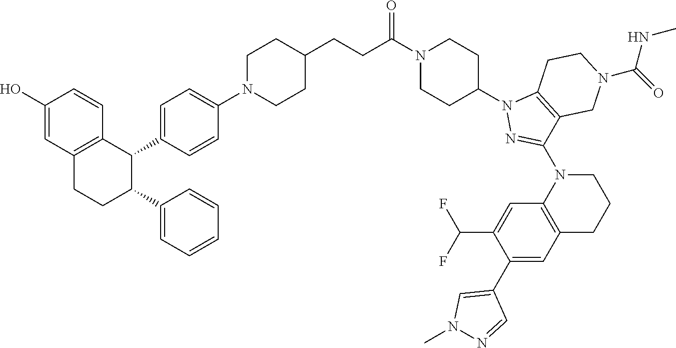 [SMILES: CNC(=O)N1CCc2c(c(N3CCCc4cc(-c5cnn(C)c5)c(C(F)F)cc43)nn2C2CCN(C(=O)CCC3CCN(c4ccc([C@H]5c6ccc(O)cc6CC[C@H]5c5ccccc5)cc4)CC3)CC2)C1] |
| 8 | 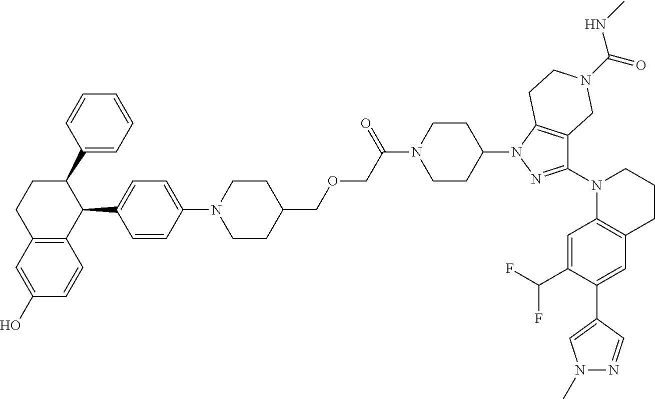 [SMILES: CNC(=O)N1CCc2c(c(N3CCCc4cc(-c5cnn(C)c5)c(C(F)F)cc43)nn2C2CCN(C(=O)COCC3CCN(c4ccc([C@H]5c6ccc(O)cc6CC[C@H]5c5ccccc5)cc4)CC3)CC2)C1] |
| 9 | 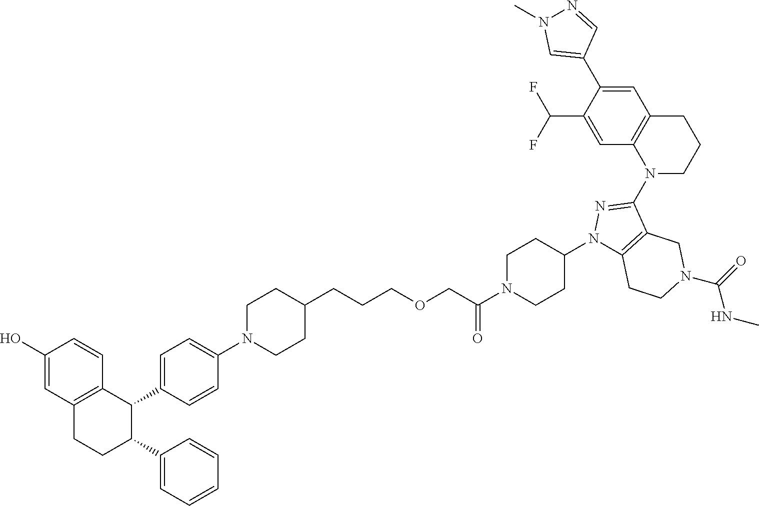 [SMILES: CNC(=O)N1CCc2c(c(N3CCCc4cc(-c5cnn(C)c5)c(C(F)F)cc43)nn2C2CCN(C(=O)COCCCC3CCN(c4ccc([C@H]5c6ccc(O)cc6CC[C@H]5c5ccccc5)cc4)CC3)CC2)C1] |
| 10 | 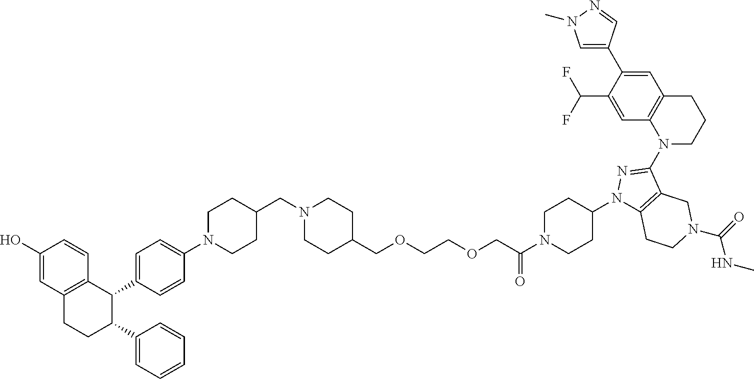 [SMILES: CNC(=O)N1CCc2c(c(N3CCCc4cc(-c5cnn(C)c5)c(C(F)F)cc43)nn2C2CCN(C(=O)COCCOCC3CCN(CC4CCN(c5ccc([C@H]6c7ccc(O)cc7CC[C@H]6c6ccccc6)cc5)CC4)CC3)CC2)C1] |
| 11 | 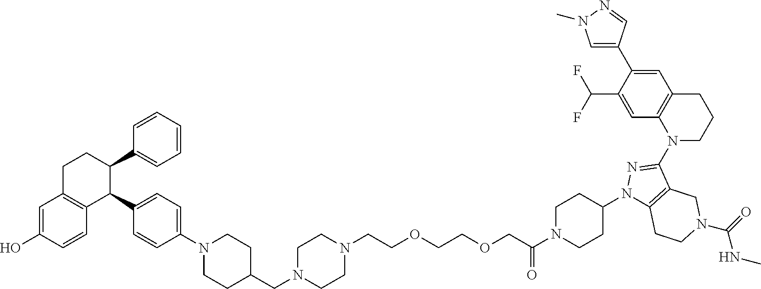 [SMILES: CNC(=O)N1CCc2c(c(N3CCCc4cc(-c5cnn(C)c5)c(C(F)F)cc43)nn2C2CCN(C(=O)COCCOCCN3CCN(CC4CCN(c5ccc([C@H]6c7ccc(O)cc7CC[C@H]6c6ccccc6)cc5)CC4)CC3)CC2)C1] |
| 12 | 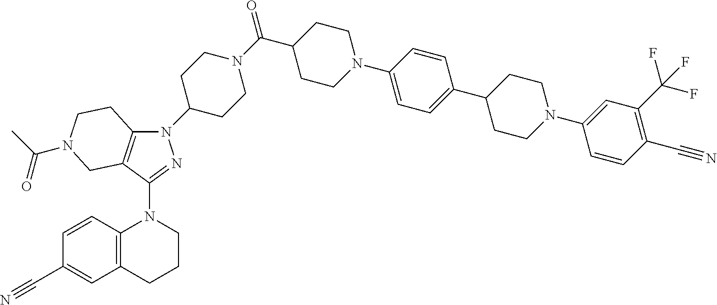 [SMILES: CC(=O)N1CCc2c(c(N3CCCc4cc(C#N)ccc43)nn2C2CCN(C(=O)C3CCN(c4ccc(C5CCN(c6ccc(C#N)c(C(F)(F)F)c6)CC5)cc4)CC3)CC2)C1] |
| 13 | 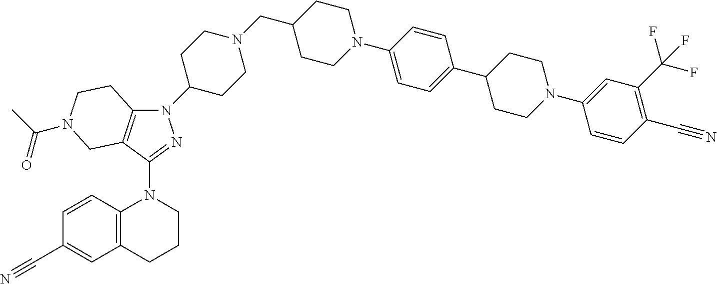 [SMILES: CC(=O)N1CCc2c(c(N3CCCc4cc(C#N)ccc43)nn2C2CCN(CC3CCN(c4ccc(C5CCN(c6ccc(C#N)c(C(F)(F)F)c6)CC5)cc4)CC3)CC2)C1] |
| 14 | 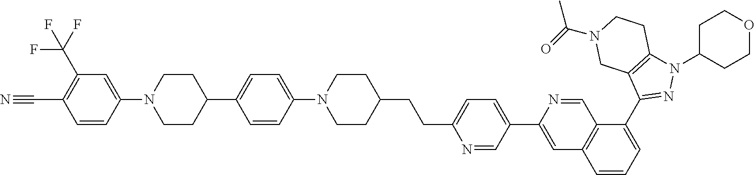 [SMILES: CC(=O)N1CCc2c(c(-c3cccc4cc(-c5ccc(CCC6CCN(c7ccc(C8CCN(c9ccc(C#N)c(C(F)(F)F)c9)CC8)cc7)CC6)nc5)ncc34)nn2C2CCOCC2)C1] |
| 15 | 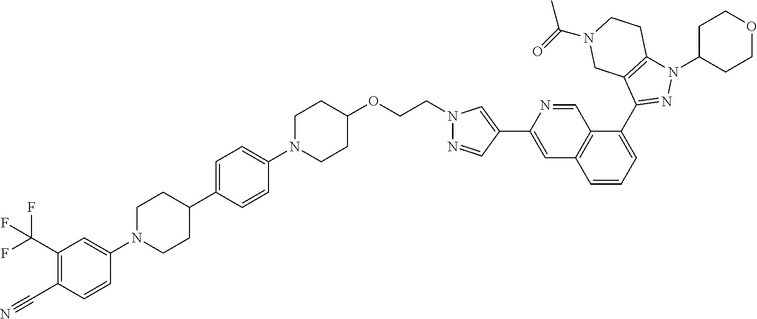 [SMILES: CC(=O)N1CCc2c(c(-c3cccc4cc(-c5cnn(CCOC6CCN(c7ccc(C8CCN(c9ccc(C#N)c(C(F)(F)F)c9)CC8)cc7)CC6)c5)ncc34)nn2C2CCOCC2)C1] |
| 16 | 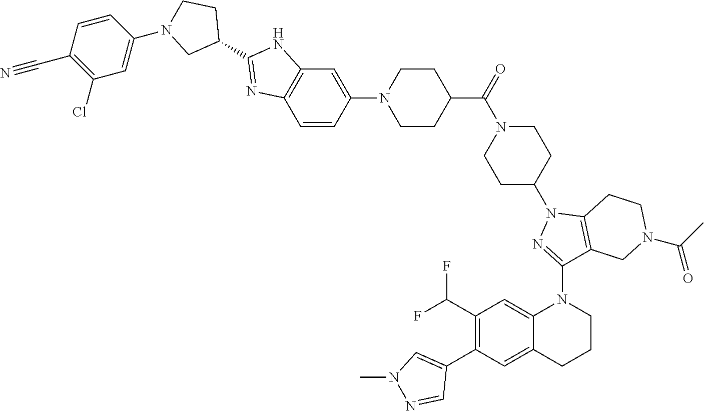 [SMILES: CC(=O)N1CCc2c(c(N3CCCc4cc(-c5cnn(C)c5)c(C(F)F)cc43)nn2C2CCN(C(=O)C3CCN(c4ccc5nc([C@H]6CCN(c7ccc(C#N)c(Cl)c7)C6)[nH]c5c4)CC3)CC2)C1] |
| 17 | 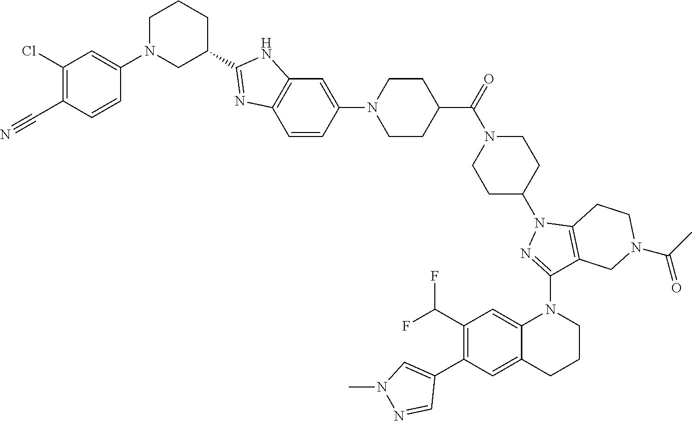 [SMILES: CC(=O)N1CCc2c(c(N3CCCc4cc(-c5cnn(C)c5)c(C(F)F)cc43)nn2C2CCN(C(=O)C3CCN(c4ccc5nc([C@H]6CCCN(c7ccc(C#N)c(Cl)c7)C6)[nH]c5c4)CC3)CC2)C1] |
| 18 | 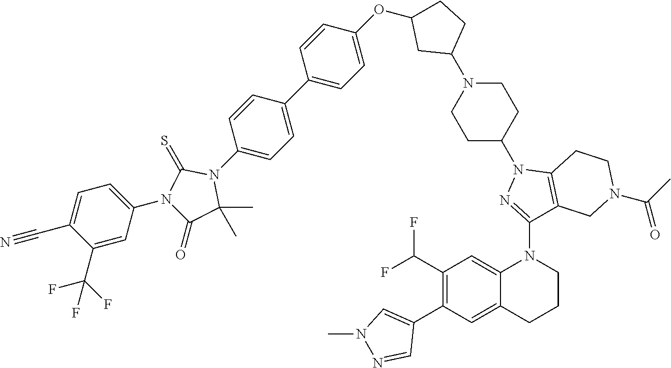 [SMILES: CC(=O)N1CCc2c(c(N3CCCc4cc(-c5cnn(C)c5)c(C(F)F)cc43)nn2C2CCN(C3CCC(Oc4ccc(-c5ccc(N6C(=S)N(c7ccc(C#N)c(C(F)(F)F)c7)C(=O)C6(C)C)cc5)cc4)C3)CC2)C1] |
| 19 | 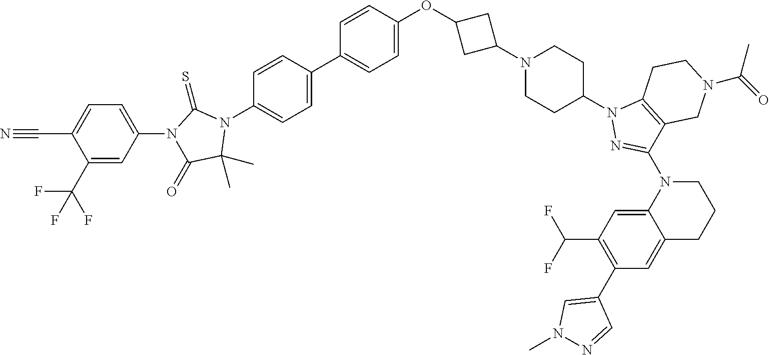 [SMILES: CC(=O)N1CCc2c(c(N3CCCc4cc(-c5cnn(C)c5)c(C(F)F)cc43)nn2C2CCN(C3CC(Oc4ccc(-c5ccc(N6C(=S)N(c7ccc(C#N)c(C(F)(F)F)c7)C(=O)C6(C)C)cc5)cc4)C3)CC2)C1] |
| 20 | 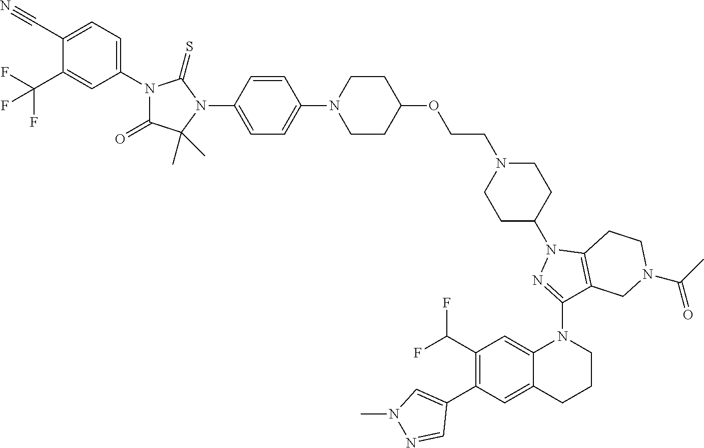 [SMILES: CC(=O)N1CCc2c(c(N3CCCc4cc(-c5cnn(C)c5)c(C(F)F)cc43)nn2C2CCN(CCOC3CCN(c4ccc(N5C(=S)N(c6ccc(C#N)c(C(F)(F)F)c6)C(=O)C5(C)C)cc4)CC3)CC2)C1] |
| 21 | 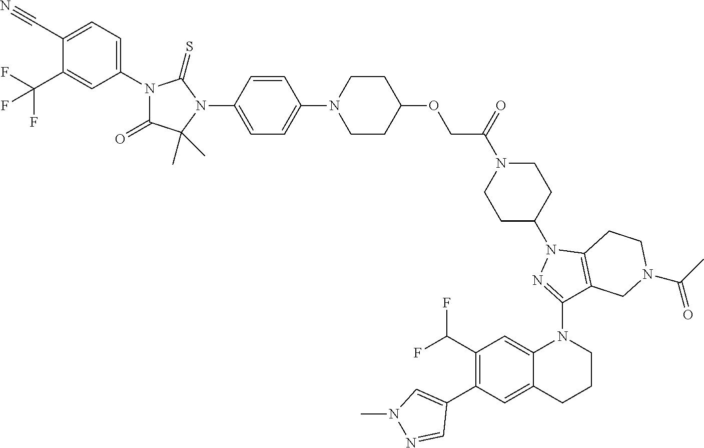 [SMILES: CC(=O)N1CCc2c(c(N3CCCc4cc(-c5cnn(C)c5)c(C(F)F)cc43)nn2C2CCN(C(=O)COC3CCN(c4ccc(N5C(=S)N(c6ccc(C#N)c(C(F)(F)F)c6)C(=O)C5(C)C)cc4)CC3)CC2)C1] |

### Further Forms of Compounds

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound disclosed herein with an acid. In some embodiments, the compound disclosed herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (− L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (− L); salicylic acid; sebacic acid; stearic acid; succinic acid; sulfuric acid; tartaric acid (+ L); thiocyanic acid; toluenesulfonic acid (p); and undecylenic acid.

In some embodiments, pharmaceutically acceptable salts are obtained by reacting a compound disclosed herein with a base. In some embodiments, the compound disclosed herein is acidic and is reacted with abase. In such situations, an acidic proton of the compound disclosed herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), or pharmaceutically acceptable salts of compounds having the structure disclosed herein, as well as active metabolites of these compounds having the same type of activity.

In some embodiments, sites on the organic radicals (e.g. alkyl groups, aromatic rings) of compounds disclosed herein are susceptible to various metabolic reactions. Incorporation of appropriate substituents on the organic radicals will reduce, minimize, or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the compounds disclosed herein possess one or more stereocenters and each stereocenter exists independently in either the R or S configuration. In some embodiments, the compound disclosed herein exists in the R configuration. In some embodiments, the compound disclosed herein exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds disclosed herein are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically, or therapeutically active form of the compound. In certain embodiments, a prodrug is enzymatically metabolized by one or more steps or processes to the biologically, pharmaceutically, or therapeutically active form of the compound.

Prodrugs of the compounds described herein include, but are not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

### Ternary Complexes

In one aspect, described herein is a stable ternary complex comprising:
a. one or more disease-dependent proteins (DDPs);
b. a disease protein (DP); and
c. heterobifunctional conditional inhibitor compound of Formula (I):

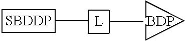

wherein:
SBDDP is a silent binder of a disease-dependent protein (DDP); L is an optional linker; and BDP is a binder of a disease protein (DP); wherein DDP and DP are present in a cell of interest (COI) and the relative abundance of the DP in the COI is greater than the relative abundance of the DDP in the COI.

In some embodiments, DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3.

In some embodiments, DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

In some embodiments, DDP is a human bromodomain-containing protein (BRD).

In some embodiments, DDP is a human bromodomain-containing protein that is a Group Ia BRD, Group Ib BRD, Group II BRD, Group IIIa BRD, Group IIIb BRD, Group 1111 BRD, Group IV BRD, Group VI BRD, Group VI BRD, Group VIII BRD, or group IX BRD; wherein:
the Group Ia BRD is selected from PCAF, GCN5L2, p300/CBP, TAF1 and TAF1L;
the Group Ib BRD is selected from BRPF1A/B (BR140), BRPF2 (BRD1) BRPF3 and BRD8 (SMAP);
the Group II BRD is selected from histone methyltransferases ASHIL and MLL;
the Group IIIa BRD is selected from chromatin remodeling factors SMARCA2 (BRM), SMARCA4 (BRG1), BRD7, BRD9 and PBRM1 (polybromo);
the Group IIIb BRD is selected from ISWI family chromatin remodeling factors BAZ1A (ACF1), BAZ1B (WSTF, William-Beuren syndrome transcription factor), BAZ2A, BAZ2B, BPTF and CECR2;
the Group IV BRD is selected from ATPase family AAA domain-containing proteins ATAD2 and ATAD2B;
the Group VI BRD is selected from Tripartite-motif-containing (TRIM) family proteins TRIM24 (TIF1α), TRIM28 (TIF1β, KAP1), TRIM33 (TIF1γ) and TRIM66 (TIF1δ);
the Group VIII BRD is selected from speckled protein (SP) family proteins SP100, SP110, SP140 and SP140L;
the Group VIII BRD is selected from transcriptional corepressors ZMYND8 and ZMYDN11; and the Group IX is selected from WD-repeat proteins BRWD1 (WDR9), BRWD3 and PHIP (WDR11).

In some embodiments, DDP is a BRD selected from PCAF, GCN5L2, p300/CBP, TAF1, TAF1L, BRPF1A/B (BR140), BRPF2 (BRD1) BRPF3, BRD8 (SMAP), chromatin remodeling factors SMARCA2 (BRM), SMARCA4 (BRG1), BRD7, BRD9, PBRM1 (polybromo), BAZ1A (ACF1), BAZ1B (WSTF, William-Beuren syndrome transcription factor), BAZ2A, BAZ2B, BPTF, CECR2, ATAD2, ATAD2B, Tripartite-motif-containing (TRIM) family proteins TRIM24 (TIF1α), TRIM28 (TIF1β, KAP1), TRIM33 (TIF1γ), TRIM66 (TIF1δ), WD-repeat proteins BRWD1 (WDR9), BRWD3 and PHIP (WDR11).

In another aspect, described here is a stable ternary complex comprising: CBP/p300; a disease protein (DP); and a heterobifunctional conditional inhibitor compound of Formula (II):

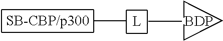

wherein: SB-CBP/p300 is a silent binder of CBP/p300; L is an optional linker; and BDP is a binder of a disease protein (DP); wherein CBP/p300 and DP are present in a cell of interest (COI) and the relative abundance of the DP in the COI is greater than the relative abundance of CBP/p300 in the COI.

In some embodiments, DP is a nuclear hormone receptor protein, G-protein coupled receptor, epidermal growth factor receptor, RAS protein, or transcription factor.

In some embodiments, DP is androgen receptor (AR), estrogen receptor (ER), progesterone receptor (PR), glucocorticoid receptor (GR), mineralocorticoid receptor (MR), angiotensin receptor, apelin receptor, bombesin receptor, bradykinin receptor, calcitonin receptor, chemokine receptor, cholecytokinin receptor, corticotropic-releasing factor receptor, galanin receptor, ghrelin receptor, glucagon receptor, glycoprotein hormone receptor, gonadotropin-releasing hormone receptor, kisspeptin receptor, melanocortin receptor, motilin receptor, neuromedin U receptor, neuropeptide FF/AF receptor, neuropeptide S receptor, neuropeptide W/B receptor, neuropeptide Y receptor, opioid receptor, orexin receptor, parathyroid hormone receptor, prokineticin receptor, prolactin-releasing peptide receptor, QRFP receptor, relaxin family peptide receptor, somatostatin receptor, tachykinin receptor, thyrotropin-releasing hormone receptor, urotensin receptor, vasopressin, oxytocin receptor, vasoactive intestinal peptide receptor, PACAP receptor, HER1, HER2, HER3, HER4, KRAS, KRAS-G12C, HRAS, NRAS, or BCL6.

In some embodiments, the DP is androgen receptor (AR), estrogen receptor (ER), progesterone receptor (PR), glucocorticoid receptor (GR), mineralocorticoid receptor (MR), retinoic acid receptor-related orphan nuclear receptor γ (RORγ), apelin receptor, bombesin receptor, bradykinin receptor, chemokine receptor, cholecytokinin receptor, chymase, gonadotropin-releasing hormone receptor, indoleamine 2,3-dioxygenase 1 (IDO1), kisspeptin receptor, neuromedin U receptor, neuropeptide FF/AF receptor, neuropeptide S receptor, neuropeptide W/B receptor, neuropeptide Y receptor, somatostatin receptor, tachykinin receptor, HER1, HER2, HER3, HER4, KRAS, KRAS-G12C, HRAS, NRAS, or BCL6. In some embodiments, DP is androgen receptor (AR) or estrogen receptor (ER).

In some embodiments, DP is androgen receptor (AR).

In some embodiments, DP is androgen receptor (AR) or DP is estrogen receptor (ER) and the heterobifunctional conditional inhibitor compound is a compound disclosed herein.

In yet another aspect, described herein is a stable ternary complex comprising: CBP/p300; Androgen receptor (AR) or Estrogen receptor (ER); and heterobifunctional conditional inhibitor compound described herein; wherein CBP/p300 and DP are present in a cell of interest (COI) and the relative abundance of the DP in the COI is greater than the relative abundance of CBP/p300 in the COI.

### Certain Terminology

Unless otherwise stated, the following terms used in this application have the definitions given below. The use of the term “including” as well as other forms, such as “include”, “includes,” and “included,” is not limiting.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

The term “acyl,” as used herein refers to the group —C(═O)—R, where R is alkyl, alkenyl, alkynyl, aryl, cycloalkyl, heteroaryl, heterocycle, or any other moiety were the atom attached to the carbonyl is carbon. An “acetyl” group refers to a —C(═O)CH₃ group.

The term “alkenyl,” as used herein refers to a straight-chain or branched-chain hydrocarbon radical having one or more double bonds and containing from 2 to 20 carbon atoms. In certain embodiments, alkenyl includes 2 to 6 carbon atoms. The term “alkenylene” refers to a divalent alkenyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkoxy” refers to a (alkyl)-O— group, where alkyl is as defined herein. In some embodiments, the alkoxy group is a C₁-C₆alkoxy, which refers to a (C₁-C₆alkyl)-O— group. Examples of alkyl groups include methoxy, ethoxy, n-propoxy, isopropoxy, n-butoxy, iso-butoxy, sec-butoxy, tert-butoxy, and the like.

An “alkyl” group refers to an aliphatic hydrocarbon group. In some embodiments, the alkyl is a straight-chain or branched-chain aliphatic hydrocarbon group containing from 1 to 20 carbon atoms. In certain embodiments, alkyl includes 1 to 10 carbon atoms. In further embodiments, the alkyl includes 1 to 8 carbon atoms. Examples of alkyl radicals include methyl, ethyl, n-propyl, isopropyl, n-butyl, isobutyl, sec-butyl, tert-butyl, pentyl, iso-amyl, hexyl, octyl, nonyl, and the like. In some embodiments, an alkyl is a C₁-C₆alkyl. In one aspect the alkyl is methyl, ethyl, propyl, iso-propyl, n-butyl, iso-butyl, sec-butyl, or t-butyl. The term “alkylene” refers to a divalent alkyl, such as methylene (—CH₂—). In some embodiments, an alkylene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like.

The term “amino,” as used herein refers to —NRR′, wherein R and R′ are independently selected from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted. In one aspect, “amino” as used herein refers to an —NH₂ group.

The term “alkynyl,” as used herein refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH. The term “alkynylene” refers to a carbon-carbon triple bond attached at two positions such as ethynylene (—C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The term “aromatic” refers to a planar ring having a delocalized it-electron system containing 4n+2 it electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

The term “aryl” as used herein means a carbocyclic aromatic system containing one, two or three rings wherein such polycyclic ring systems are fused together. The term “aryl” embraces aromatic groups such as phenyl, naphthyl, anthracenyl, and phenanthryl. In one aspect, aryl is phenyl or a naphthyl. In some embodiments, an aryl is a phenyl. In some embodiments, an aryl is a phenyl, naphthyl, indanyl, indenyl, or tetrahyodronaphthyl. In some embodiments, an aryl is a C₆-C₁₀aryl. Depending on the structure, an aryl group is a monoradical or a diradical (i.e., an arylene group).

The terms “benzo” and “benz,” as used herein refer to fused bicyclic or polyclic ring system that is formed with benzene as one of the rings. Examples include benzofuran, benzothiophene, and benzimidazole.

The term “cycloalkyl,” as used herein refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In some embodiments, cycloalkyl groups include groups having from 3 to 10 ring atoms. In certain embodiments, said cycloalkyl will comprise from 5 to 7 carbon atoms. In certain embodiments, said cycloalkyl will comprise from 3 to 6 carbon atoms. Examples of such cycloalkyl groups include cyclopropyl, cyclobutyl, cyclopentyl, cyclohexyl, cycloheptyl, tetrahydronaphthyl, indanyl, octahydronaphthyl, 2,3-dihydro-1H-indenyl, adamantly, and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane. In some embodiments, a cycloalkyl is a C₃-C₆cycloalkyl. In some embodiments, a cycloalkyl is a C₃-C₄cycloalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofiranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. In some embodiments, the term “heteroaryl,” as used herein refers to a 3 to 15 membered unsaturated heteromonocyclic ring, or a fused monocyclic, bicyclic, or tricyclic ring system in which at least one of the fused rings is aromatic, which contains at least one atom selected from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, imidazolyl, pyrazolyl, pyridyl, pyrimidinyl, pyrazinyl, pyridazinyl, triazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuranyl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl, and the like. Exemplary tricyclic heterocyclic groups include carbazolyl, benzidolyl, phenanthrolinyl, dibenzofuranyl, acridinyl, phenanthridinyl, xanthenyl, and the like. In some embodiments, a heteroaryl contains 0-4 N atoms in the ring. In some embodiments, a heteroaryl contains 1-4 N atoms in the ring. In some embodiments, a heteroaryl contains 1 O atom in the ring. In some embodiments, a heteroaryl contains 1 S atom in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen and sulfur. In some embodiments, the term “heterocycloalkyl” as used herein each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 1-2 N atoms in the ring. In some embodiments, a heterocycloalkyl contains 1-2 O atoms. In some embodiments, a heterocycloalkyl contains 1 S atom. In some embodiments, a heterocycloalkyl contains 0-2 N atoms, 0-2 O atoms and 0-1 S atoms in the ring.

The term “carbamate,” as used herein refers to an ester of carbamic acid (—NHCOO—) which may be attached to the parent molecular moiety from either the nitrogen or acid end, and which may be optionally substituted as defined herein.

The term “carboxyl” or “carboxy,” as used herein, refers to —C(═O)OH or the corresponding “carboxylate” anion, such as is in a carboxylic acid salt.

The term “cyano,” as used herein refers to —CN.

The term “ester,” as used herein refers to a carboxy group bridging two moieties linked at carbon atoms.

The term “ether,” as used herein refers to an oxy group bridging two moieties linked at carbon atoms.

The term “halo,” or “halogen,” as used herein refers to fluorine, chlorine, bromine, or iodine. In some embodiments, halo is fluoro, chloro, or bromo.

The term “haloalkyl,” as used herein refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—), and the like. In one aspect, a haloalkyl is a C₁-C₆haloalkyl. In another aspect, a haloalkyl is a C₁-C₄haloalkyl.

The term “haloalkoxy,” as used herein refers to a haloalkyl group attached to the parent molecular moiety through an oxygen atom. In one aspect, the haloalkoxy is a C₁-C₆haloalkoxy, which refers to a (C₁-C₆haloalkyl)-O— group. In another aspect, the haloalkoxy is a C₁-C₄haloalkoxy, which refers to a (C₁-C₄haloalkyl)-O— group.

The term “heteroalkyl” refers to an alkyl wherein 1 or more carbon atoms are replaced with a heteroatom. In some embodiments, “heteroalkyl” refers to an alkyl wherein 1 or more carbon atoms are replaced with one or more heteroatoms that are independently selected from NH, —N(alkyl), 0, S, S(═O) and S(═O)₂. The attachment of the heteroatom(s) to the remainder of the compound is at a carbon atoms of the heteroalkyl. In some embodiments, up to two heteroatoms may be consecutive, such as, for example, —CH₂—NH—OCH₃. In some embodiments, “heteroalkyl” is an “alkoxyalkyl”, “alkylthioalkyl”, or “alkylaminoalkyl”. “Alkoxyalkyl” refers to an alkyl in which one hydrogen atom is replaced by an alkoxy group, as defined herein. In some embodiments, an alkoxyalkyl is a (C₁-C₆alkoxy)-C₁-C₆alkyl. Typical alkoxyalkyl groups include, but are not limited to, —CH₂OCH₃, —CH₂CH₂OCH₃, —CH₂CH₂CH₂OCH₃, —CH₂CH₂CH₂CH₂OCH₃, —CH₂OCH₂CH₃, —CH₂CH₂OCH₂CH₃, —CH₂CH₂CH₂OCH₂CH₃, —CH₂CH₂CH₂CH₂OCH₂CH₃, and the like. “Alkylthioalkyl” refers to an alkyl in which one hydrogen atom is replaced by an alkylthio group, as defined herein. In some embodiments, an alkoxyalkyl is a (C₁-C₆ alkylthio)-C₁-C₆alkyl. Typical alkoxyalkyl groups include, but are not limited to, —CH₂SCH₃, —CH₂CH₂SCH₃, —CH₂CH₂CH₂SCH₃, —CH₂CH₂CH₂CH₂SCH₃, —CH₂SCH₂CH₃, —CH₂CH₂SCH₂CH₃, —CH₂CH₂CH₂SCH₂CH₃, —CH₂CH₂CH₂CH₂SCH₂CH₃, and the like. “Alkylaminoalkyl” refers to an alkyl in which one hydrogen atom is replaced by an alkylamino group, as defined herein. In some embodiments, an alkoxyalkyl is a (C₁-C₆alkylamino)-C₁-C₆alkyl. Typical alkoxyalkyl groups include, but are not limited to, —CH₂NHCH₃, —CH₂CH₂NHCH₃, —CH₂CH₂CH₂NHCH₃, —CH₂CH₂CH₂CH₂NHCH₃, —CH₂NHCH₂CH₃, —CH₂CH₂NHCH₂CH₃, —CH₂CH₂CH₂NHCH₂CH₃, —CH₂CH₂CH₂CH₂NHCH₂CH₃, and the like.

The term “hydroxy,” or “hydroxyl,” as used herein refers to —OH.

The term “hydroxyalkyl,” as used herein refers to a hydroxy group attached to the parent molecular moiety through an alkyl group. In some embodiments, a hydroxyalkyl is a C₁-C₄hydroxyalkyl. Typical hydroxyalkyl groups include, but are not limited to, —CH₂OH, —CH₂CH₂OH, —CH₂CH₂CH₂OH, —CH₂CH₂CH₂CH₂OH, and the like.

The phrase “linear chain of atoms” refers to the longest straight chain of atoms independently selected from carbon, nitrogen, oxygen and sulfur.

The term “nitro,” as used herein refers to —NO₂.

The term “oxo,” as used herein refers to ═O.

The terms “sulfonate,” “sulfonic acid,” and “sulfonic,” as used herein refer the —SO₃H group and its anion as the sulfonic acid is used in salt formation.

The term “sulfanyl,” as used herein refers to —S—.

The term “sulfinyl,” as used herein refers to —S(═O)—.

The term “sulfonyl,” as used herein refers to a —S(═O)₂—, —S(═O)₂R, or —S(═O)₂R— group, with R as defined herein.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

In some embodiments, the term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” or “disorder” as used herein refers to any condition that impairs the normal functioning of the body, such as a functional abnormality or disturbance that impairs normal functioning.

The term “combination therapy” means the administration of two or more therapeutic agents to treat a therapeutic condition or disorder described in the present disclosure.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, “treating,” “treatment,” and the like means ameliorating a disease, so as to reduce, ameliorate, or eliminate its cause, its progression, its severity, or one or more of its symptoms, or otherwise beneficially alter the disease in a subject. In certain embodiments, reference to “treating” or “treatment” of a subject at risk for developing a disease, or at risk of disease progression to a worse state, is intended to include prophylaxis. Prevention of a disease may involve complete protection from disease or may involve prevention of disease progression. Prevention of diseases may also mean prevention of progression of a disease to a later stage of the disease.

The term “patient” is generally synonymous with the term “subject” and includes all mammals including humans. Examples of patients include humans, non-human primates such as chimpanzees, and other apes and monkey species; livestock such as cows, goats, sheep, pigs, and rabbits, and companion animals such as dogs, cats, rabbits, and horses. Preferably, the patient is a human.

### Pharmaceutical Compositions and Formulations

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tableting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound provided herein in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20th Edition, 2000, Lippincott Williams & Wilkins, (Editors: Gennaro et. al.).

The compounds provided herein, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds provided herein or a salt, solvate, or hydrate thereof can be used as active ingredients in pharmaceutical compositions. The term “active ingredient”, defined in the context of a “pharmaceutical composition”, refers to a component of a pharmaceutical composition that provides the primary pharmacological effect, as opposed to an “inactive ingredient” which would generally be recognized as providing no pharmaceutical benefit.

The dose when using the compounds provided herein can vary within wide limits and as is customary and is known to the physician or other clinician, it is to be tailored to the individual conditions in each individual case. It depends, for example, on the nature and severity of the illness to be treated, on the condition of the patient, on the compound employed or on whether an acute or chronic disease state is treated, or prophylaxis conducted, or on whether further active compounds are administered in addition to the compounds provided herein. Representative doses include, but are not limited to, about 0.001 mg to about 5000 mg, about 0.001 mg to about 2500 mg, about 0.001 mg to about 1000 mg, about 0.001 mg to about 500 mg, about 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3, or 4 doses. Depending on the individual and as deemed appropriate from the healthcare provider it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated, or prophylaxis conducted, or on whether further active compounds are administered in addition to the compounds provided herein and as part of a drug combination. The dosage regimen for treating a disease condition with the compounds and/or compositions provided herein is selected in accordance with a variety of factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods provided herein.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents.

Aqueous formulations suitable for oral use can be prepared by dissolving or suspending the active component in water and adding suitable colorants, flavors, stabilizing and thickening agents, as desired.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the compounds provided herein may be formulated as ointments, creams, or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

### Methods of Dosing and Treatment Regimens

In one embodiment, the compounds disclosed herein, or a pharmaceutically acceptable salt thereof, are used in the preparation of medicaments for the treatment of diseases or conditions in a mammal. Methods for treating any of the diseases or conditions described herein in a mammal in need of such treatment, involves administration of pharmaceutical compositions that include at least one compound disclosed herein, or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound disclosed herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-2000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound disclosed herein, or a pharmaceutically acceptable salt thereof, described herein are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound disclosed herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In another aspect, described herein is a method of selectively inhibiting the activity of a disease-dependent protein (DDP) in a cell of interest (COI) of a mammal comprising administering a heterobifunctional compound described herein, or a pharmaceutically acceptable salt or solvate thereof, wherein the heterobifunctional compound inhibits the activity of the DDP in the COI but does not inhibit the activity of the DDP in cells expressing the DDP and not expressing the DP. In some embodiments, the DP is overexpressed, overactive or both overexpressed and overactive in the COI.

In some embodiments, DP is a nuclear hormone receptor protein, G-protein coupled receptor, epidermal growth factor receptor, RAS protein, or transcription factor. In some embodiments, DP is androgen receptor (AR), estrogen receptor (ER), progesterone receptor (PR), glucocorticoid receptor (GR), mineralocorticoid receptor (MR), angiotensin receptor, apelin receptor, bombesin receptor, bradykinin receptor, calcitonin receptor, chemokine receptor, cholecytokinin receptor, corticotropic-releasing factor receptor, galanin receptor, ghrelin receptor, glucagon receptor, glycoprotein hormone receptor, gonadotropin-releasing hormone receptor, kisspeptin receptor, melanocortin receptor, motilin receptor, neuromedin U receptor, neuropeptide FF/AF receptor, neuropeptide S receptor, neuropeptide W/B receptor, neuropeptide Y receptor, opioid receptor, orexin receptor, parathyroid hormone receptor, prokineticin receptor, prolactin-releasing peptide receptor, QRFP receptor, relaxin family peptide receptor, somatostatin receptor, tachykinin receptor, thyrotropin-releasing hormone receptor, urotensin receptor, vasopressin, oxytocin receptor, vasoactive intestinal peptide receptor, PACAP receptor, HER1, HER2, HER3, HER4, KRAS, KRAS-G12C, HRAS, NRAS, or BCL6.

In some embodiments, the DP is androgen receptor (AR), estrogen receptor (ER), progesterone receptor (PR), glucocorticoid receptor (GR), mineralocorticoid receptor (MR), retinoic acid receptor-related orphan nuclear receptor γ (RORγ), apelin receptor, bombesin receptor, bradykinin receptor, chemokine receptor, cholecytokinin receptor, chymase, gonadotropin-releasing hormone receptor, indoleamine 2,3-dioxygenase 1 (IDO1), kisspeptin receptor, neuromedin U receptor, neuropeptide FF/AF receptor, neuropeptide S receptor, neuropeptide W/B receptor, neuropeptide Y receptor, somatostatin receptor, tachykinin receptor, HER1, HER2, HER3, HER4, KRAS, KRAS-G12C, HRAS, NRAS, or BCL6.

In some embodiments, DP is AR or ER. In some embodiments, DP is androgen receptor (AR).

In some embodiments, DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3.

In some embodiments, DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

In some embodiments, DDP is a BRD selected from PCAF, GCN5L2, p300/CBP, TAF1, TAF1L, BRPF1A/B (BR140), BRPF2 (BRD1) BRPF3, BRD8 (SMAP), chromatin remodeling factors SMARCA2 (BRM), SMARCA4 (BRG1), BRD7, BRD9, PBRM1 (polybromo), BAZ1A (ACF1), BAZ1B (WSTF, William-Beuren syndrome transcription factor), BAZ2A, BAZ2B, BPTF, CECR2, ATAD2, ATAD2B, Tripartite-motif-containing (TRIM) family proteins TRIM24 (TIF1α), TRIM28 (TIF1β, KAP1), TRIM33 (TIF1γ), TRIM66 (TIF1δ), WD-repeat proteins BRWD1 (WDR9), BRWD3 and PHIP (WDR11).

In some embodiments, the DDP is CREB-binding protein (CBP)/p300.

In another aspect, described herein is a method of treating cancer in a mammal comprising administering a therapeutically effective amount of to the mammal a heterobifunctional compound described herein, or a pharmaceutically acceptable salt or solvate thereof.

In some embodiments, the cancer is a hormone dependent cancer.

In some embodiments, the cancer is adenomas and neoplasms of the prostate, tumor cells expressing the androgen receptor, tumor cells expressing the estrogen receptor, prostate cancer, breast cancer, endometrial cancer, or uterine cancer.

In certain instances, it is appropriate to administer at least one compound disclosed herein, or a pharmaceutically acceptable salt thereof, in combination with one or more other therapeutic agents.

As used above, and throughout the description of the invention, the following abbreviations, unless otherwise indicated, shall be understood to have the following meanings:
ACN or MeCN acetonitrile
Ac acetyl
Ac₂O acetic anhydride
AcOH acetic acid
AIBN azobisisobutyronitrile
BINAP 2,2′-bis(diphenylphosphino)-1,1′-binaphthalene
Bn benzyl
BOC or Boc tert-butyl carbamate
Boc₂O di-tert-butyl decarbonate or Boc anhydride
BPO benzoyl peroxide
BrettPhos Pd G3 [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate
t-Bu tert-butyl
C Phos Pd G3 [(2-Dicyclohexylphosphino-2′,6′-bis(N,N-dimethylamino)-1,1′-biphenyl)-2-(2′-amino-1,1′-biphenyl)]palladium(II) methanesulfonate
Cy cyclohexyl
DBA or dba dibenzylideneacetone
DBU 1,8-diazabicyclo[5.4.0]undec-7-ene
DCE dichloroethane (ClCH₂CH₂Cl)
DCM dichloromethane (CH₂Cl₂)
DIPEA or DIEA diisopropylethylamine
DMAP 4-(N,N-dimethylamino)pyridine
DMF dimethylformamide
DMA N,N-dimethylacetamide
DMSO dimethylsulfoxide
Dppf 1,1′-bis(diphenylphosphino)ferrocene
EEDQ 2-Ethoxy-1-ethoxycarbonyl-1,2-dihydroquinoline
eq equivalent(s)
Et ethyl
Et₂O diethyl ether
EtOH ethanol
EtOAc ethyl acetate
FA formic acid
HATU 1-[bis(dimethylamino)methylene]-1H-1,2,3-triazolo[4,5-b]pyridinium 3-oxid hexafluorophosphate
HPLC high performance liquid chromatography
H₂O water
K₂CO₃ potassium carbonate
LAH lithium aluminum anhydride
LCMS liquid chromatography mass spectrometry
Me methyl
MeOH methanol
MS mass spectrometry
MsCl mesyl chloride
Na₂CO₃ sodium carbonate
NaBH(OAc)₃ sodium triacetoxyborohydride
NaH sodium hydride
Na₂SO₄ sodium sulfate
NBS N-bromo succinamide
NMP N-methyl-pyrrolidin-2-one
NMR nuclear magnetic resonance
Pd(dppf)Cl₂ (1,1′-Bis(diphenylphosphino)ferrocene)palladium(II) dichloride
Ph phenyl
Pin pinacolato
Prep-HPLC preparative high performance liquid chromatography
i-Pr iso-propyl
RP-HPLC reverse phase-high pressure liquid chromatography
RuPhos Pd G3 (2-Dicyclohexylphosphino-2′,6′-diisopropoxy-1,1′-biphenyl)[2-(2′-amino-1,1′-biphenyl)]palladium(II) methanesulfonate
TBS tert-butyldimethylsilyl
TFA trifluoroacetic acid
TEA triethylamine
THF tetrahydrofuran
TsCl tosyl chloride
TLC thin layer chromatography

### EXAMPLES

The following examples are provided for illustrative purposes only and not to limit the scope of the claims provided herein.

Compound C was prepared according to the following procedure:

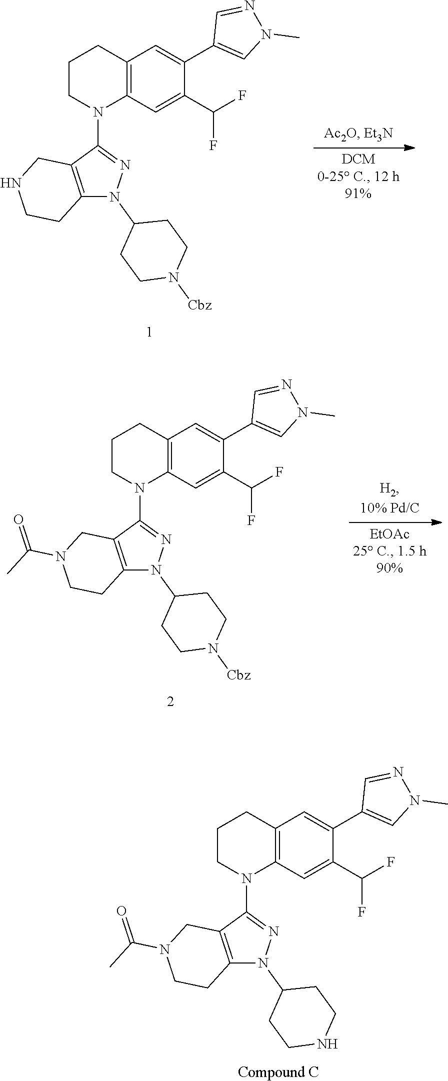

1 was prepared according to the procedure described in WO 2022/42707 A1.

To a solution of benzyl 4-[3-[7-(difluoromethyl)-6-(1-methylpyrazol-4-yl)-3,4-dihydro-2H-quinolin-1-yl]-4,5,6,7-tetrahydropyrazolo[4,3-c]pyridin-1-yl]piperidine-1-carboxylate (0.250 g, 349 μmol, 1.0 eq, TFA) in DCM (0.5 mL) was added TEA (106 mg, 1.05 mmol, 146 μL, 3.0 eq) and Ac₂O (53.5 mg, 524 μmol, 49.2 μL, 1.5 eq) at 0° C. The mixture was stirred at 25° C. for 12 h. The mixture was poured into ice-water (20 mL). The aqueous phase was extracted with dichloromethane (15 mL×3). The combined organic phase was washed with brine (10 mL×2), dried over anhydrous sodium sulfate, filtered and concentrated under vacuum. The residue was purified by prep-TLC (silica, dichloromethane/methyl alcohol=10/1). Compound benzyl 4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carboxylate (0.21 g, 316 μmol, 91% yield) was obtained as a light yellow solid. LC-MS: MS (ESI⁺): t_{R}=0.980 min, m/z=644.3 [M+H⁺]

Pd/C (0.110 g, 10% Pd on carbon, w/w) was added into a 100 mL single-necked round bottom flask under N₂, and then EtOAc (10 mL) was added at 25° C. under N₂. After addition, benzyl 4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]piperidine-1-carboxylate (0.210 g, 326 μmol, 1.0 eq) was added under N₂. The suspension was degassed under vacuum and purged with H₂ several times. The mixture was stirred under H₂ (15 psi) at 25° C. for 1.5 h. The reaction mixture was filtered and washed with MeOH (20 mL×3). The collected filtrate was concentrated to give a residue. The residue was used for the next step without further purification. Compound 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (0.15 g, 294 μmol, 90% yield) was obtained as a white solid. LC-MS: MS (ESI⁺): t_{R}=0.429 min, m/z=510.3 [M+H⁺]

### Example 1: Synthesis of 4-[4-[4-[3-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]propoxy]phenyl]-1-piperidyl]-2-(trifluoromethyl)benzonitrile (Compound 4)

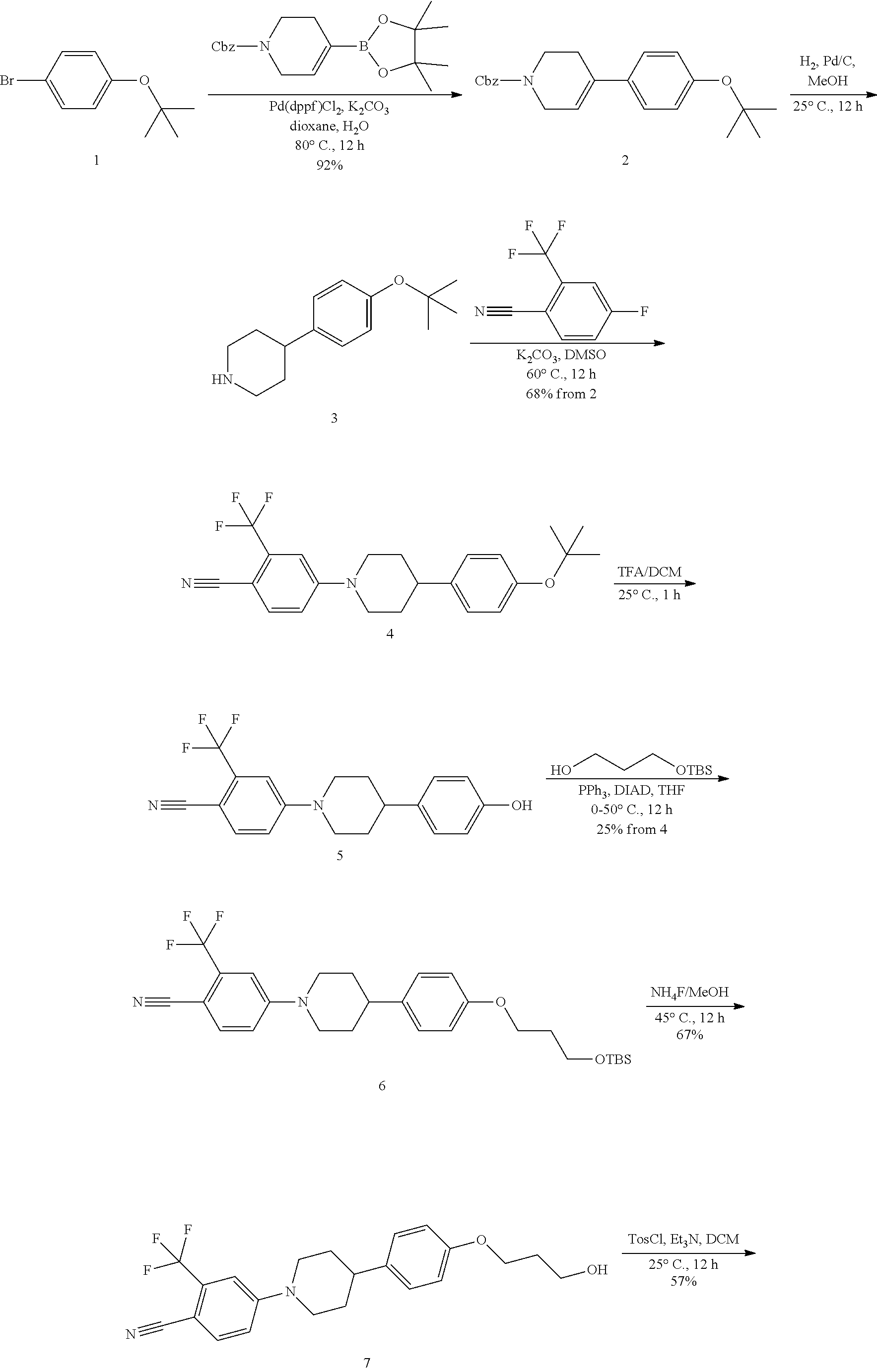

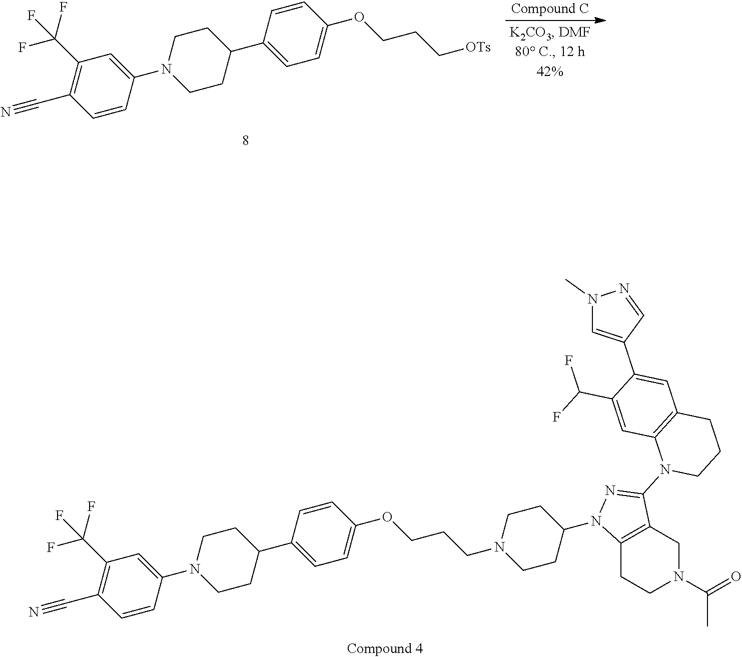 [SMILES: Cc1ccc(S(=O)(=O)OCCCOc2ccc(C3CCN(c4ccc(C#N)c(C(F)(F)F)c4)CC3)cc2)cc1 ; CC(=O)N1CCc2c(c(N3CCCc4cc(-c5cnn(C)c5)c(C(F)F)cc43)nn2C2CCN(CCCOc3ccc(C4CCN(c5ccc(C#N)c(C(F)(F)F)c5)CC4)cc3)CC2)C1]

To a solution of 1-bromo-4-tert-butoxy-benzene (5.00 g, 21.8 mmol, 1.0 eq) and benzyl 4-(4,4,5,5-tetramethyl-1,3,2-dioxaborolan-2-yl)-3,6-dihydro-2H-pyridine-1-carboxylate (11.24 g, 32.7 mmol, 1.5 eq) in dioxane (80 mL) and H₂ (16 mL) was added Pd(dppf)Cl₂ (1.60 g, 2.18 mmol, 0.1 eq) and K₂CO₃ (6.03 g, 43.7 mmol, 2.0 eq) The mixture was stirred at 80° C. for 12 h under N₂. The mixture was poured into water (50 mL). The aqueous phase was extracted with ethyl acetate (50 mL×3), dried with anhydrous sodium sulfate, filtered and concentrated under vacuum. The residue was purified by column chromatography (SiO₂, Petroleum ether/Ethyl acetate=100/1 to 20/1). Compound benzyl 4-(4-tert-butoxyphenyl)-3,6-dihydro-2H-pyridine-1-carboxylate (10.5 g, 20.1 mmol, 92% yield) was obtained as a yellow solid.

LC-MS: MS (ESI⁺): t_{R}=0.703 min, m/z=310.2 [M-55]

Pd/C (4.00 g, 10% Pd on carbon, w/w, 0.2 eq) was added into a 250 mL single-necked round bottom flask under N₂, and then MeOH (60 mL) was added at 25° C. under N₂. After addition, benzyl 4-(4-tert-butoxyphenyl)-3,6-dihydro-2H-pyridine-1-carboxylate (6.00 g, 16.4 mmol, 1.0 eq) was added under N₂. The suspension was degassed under vacuum and purged with H₂ several times. The mixture was stirred under H₂ at 25° C. for 12 h. The reaction mixture was filtered and washed with MeOH (50 mL×3). The collected filtrate was concentrated to give a residue. Compound 4-(4-tert-butoxyphenyl)piperidine (3.10 g) as a yellow oil and directly used in the next step without further purification.

LC-MS: MS (ESI⁺): t_{R}=0.470 min, m/z=234.2 [M+H⁺]

To a solution of 4-(4-tert-butoxyphenyl)piperidine (2.20 g) and 4-fluoro-2-(trifluoromethyl)benzonitrile (2.14 g, 11.3 mmol, 1.2 eq) in DMSO (30 mL) was added K₂CO₃ (3.91 g, 28.3 mmol, 3.0 eq). The mixture was stirred at 60° C. for 12 h under N₂. The mixture was poured into water (60 mL). The aqueous phase was extracted with ethyl acetate (60 mL×3), dried with anhydrous sodium sulfate, filtered and concentrated under vacuum. The residue was purified by prep-HPLC (column: Phenomenex luna C18 150×40 mm×15 um; mobile phase: [water(FA)-ACN]; gradient: 70%-100% B over 15 min). Compound t 4-[4-(4-tert-butoxyphenyl)-1-piperidyl]-2-(trifluoromethyl)benzonitrile (2.70 g, 6.37 mmol, 68% yield over two steps) was obtained as a white solid.

LC-MS: MS (ESI⁺): t_{R}=0.733 min, m/z=403.3 [M+H⁺]

To a solution of 4-[4-(4-tert-butoxyphenyl)-1-piperidyl]-2-(trifluoromethyl)benzonitrile (2.40 g, 5.96 mmol, 1.0 eq) in DCM (10 mL) was added TFA (30.7 g, 269 mmol, 45.2 eq). The mixture was stirred at 25° C. for 2 h under N₂. The mixture was concentrated to give a residue. Compound 4-[4-(4-hydroxyphenyl)-1-piperidyl]-2-(trifluoromethyl)benzonitrile (2.50 g) as a yellow solid and directly used in the next step without further purification.

LC-MS: MS (ESI⁺): t_{R}=0.622 min, m/z=347.2 [M+H⁺]

To a solution of 4-[4-(4-hydroxyphenyl)-1-piperidyl]-2-(trifluoromethyl)benzonitrile (1.00 g) and 3-[tert-butyl(dimethyl)silyl]oxypropan-1-ol (824 mg, 4.33 mmol, 1.5 eq) in toluene (20 mL) was added PPh₃ (0.30 M, 14.4 mL, 1.5 eq) and DIAD (553 mg, 3.18 mmol, 1.1 eq) at 0° C. The mixture was stirred at 25° C. for 12 h under N₂. The mixture was poured into water (50 mL). The aqueous phase was extracted with ethyl acetate (50 mL×3), dried with anhydrous sodium sulfate, filtered and concentrated under vacuum. The residue was purified by prep-HPLC (column: Phenomenex luna C18 150×40 mm×15 um; mobile phase: [water(FA)-ACN]; gradient: 90%-100% B over 15 min). Compound 4-[4-[4-[3-[tert-butyl(dimethyl)silyl]oxypropoxy]phenyl]-1-piperidyl]-2-(trifluoromethyl)benzonitrile (410 mg, 732 μmol, 25% yield over two steps) was obtained as a white solid.

LC-MS: MS (ESI⁺): t_{R}=0.820 min, m/z=519.4 [M+H⁺]

To a solution of 4-[4-[4-[3-[tert-butyl(dimethyl)silyl]oxypropoxy]phenyl]-1-piperidyl]-2-(trifluoromethyl)benzonitrile (410 mg, 732 μmol, 1.0 eq) in MeOH (5 mL) was added NH₄F (286 mg, 7.71 mmol, 10.0 eq). The mixture was stirred at 45° C. for 12 h under N₂. The mixture was poured into water (20 mL). The aqueous phase was extracted with ethyl acetate (20 mL×3), dried with anhydrous sodium sulfate, filtered and concentrated under vacuum. The residue was purified by prep-TLC (SiO₂, Petroleum ether/Ethyl acetate=2/1). Compound 4-[4-[4-(3-hydroxypropoxy)phenyl]-1-piperidyl]-2-(trifluoromethyl)benzonitrile (220 mg, 517 μmol, 67% yield) was obtained as a white solid.

LC-MS: MS (ESI⁺): t_{R}=0.633 min, m/z=405.2 [M+H⁺]

To a solution of 4-[4-[4-(3-hydroxypropoxy)phenyl]-1-piperidyl]-2-(trifluoromethyl)benzonitrile (150 mg, 371 μmol, 1.0 eq) in DCM (5 mL) was added Et₃N (113 mg, 1.11 mmol, 3.0 eq) and TsCl (106 mg, 556 μmol, 1.5 eq). The mixture was stirred at 25° C. for 12 h under N₂. The mixture was poured into iced water (20 mL). The aqueous phase was extracted with dichloromethane (15 mL×3). The combined organic phase was washed with brine (10 mL×2), dried with anhydrous sodium sulfate, filtered and concentrated under vacuum. The residue was purified by prep-TLC (SiO₂, Petroleum ether/Ethyl acetate=2/1). Compound 3-[4-[1-[4-cyano-3-(trifluoromethyl)phenyl]-4-piperidyl]phenoxy]propyl 4-methylbenzenesulfonate (125 mg, 213 μmol, 57% yield) was obtained as a yellow oil.

LC-MS: MS (ESI⁺): t_{R}=0.702 min, m/z=559.3 [M+H⁺]

To a solution of 3-[4-[1-[4-cyano-3-(trifluoromethyl)phenyl]-4-piperidyl]phenoxy]propyl 4-methylbenzenesulfonate (105 mg, 188 μmol, 1.2 eq) and 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (80 mg, 157 μmol, 1.0 eq) in DMF (2 mL) was added K₂CO₃ (43 mg, 314 μmol, 2.0 eq). The mixture was stirred at 80° C. for 12 h under N₂. The mixture was poured into water (20 mL). The aqueous phase was extracted with ethyl acetate (20 mL×3), dried with anhydrous sodium sulfate, filtered and concentrated under vacuum. The residue was purified by prep-HPLC (column: Phenomenex luna C18 150×25 mm×10 um; mobile phase: [water(FA)-ACN]; gradient: 31%-61% B over 10 min) to give 4-[4-[4-[3-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]propoxy]phenyl]-1-piperidyl]-2-(trifluoromethyl)benzonitrile (59.29 mg, 65.2 μmol, 42% yield) was obtained as a off-white solid.

¹H NMR (400 MHz, CHLOROFORM-d) 6=8.47 (s, 1H), 7.62 (d, J=8.8 Hz, 1H), 7.54 (d, J=5.6 Hz, 1H), 7.41 (d, J=6.8 Hz, 1H), 7.19-7.10 (m, 3H), 7.08-6.96 (m, 2H), 6.92-6.83 (m, 3H), 6.71-6.35 (m, 1H), 4.26 (s, 1H), 4.12 (s, 1H), 4.08-3.86 (m, 9H), 3.80-3.66 (m, 3H), 3.23-2.99 (m, 4H), 2.93-2.57 (m, 7H), 2.39-2.20 (m, 5H), 2.17 (s, 2H), 2.11 (s, 1H), 2.04-1.94 (m, 6H), 1.85-1.67 (m, 2H)

LC-MS: MS (ESI⁺): t_{R}=2.071 min m/z=896 [M+H⁺]

The compounds below were prepared in a similar manner as described in Example 1.

| | | | LC-MS |
|---|---|---|---|
| Compound | Name | ¹H NMR (CDCl₃) | [M + H⁺] |
| 2 | 4-[4-[4-[2-[2-[4-[5-acetyl-3-[7- | δ = 8.48-8.44 (m, 1H), 7.62 (d, J = 8.8 | 926.7 |
| | (difluoromethyl)-6-(1- | Hz, 1H), 7.56-7.52 (m, 1H), 7.44-7.38 | |
| | methylpyrazol-4-yl)-3,4-dihydro- | (m, 1H), 7.18-7.10 (m, 3H), 7.08-6.96 | |
| | 2H-quinolin-1-yl]-6,7-dihydro- | (m, 2H), 6.94-6.84 (m, 3H), 6.70-6.36 | |
| | 4H-pyrazolo[4,3-c]pyridin-1-yl]- | (m, 1H), 4.24 (s, 1H), 4.15-4.10 (m, | |
| | 1-piperidyl]ethoxy]ethoxy]phenyl]- | 3H), 4.02 (d, J = 12.8 Hz, 2H), 3.96 (d, J = | |
| | 1-piperidyl]-2- | 2.4 Hz, 4H), 3.90 (t, J = 5.6 Hz, 1H), | |
| | (trifluoromethyl)benzonitrile | 3.86-3.80 (m, 2H), 3.79-3.66 (m, 5H), | |
| | | 3.24-3.11 (m, 2H), 3.10-3.00 (m, 2H), | |
| | | 2.91-2.79 (m, 3H), 2.78-2.66 (m, 4H), | |
| | | 2.39-2.20 (m, 4H), 2.16 (s, 1H), 2.11- | |
| | | 2.06 (m, 2H), 2.06-1.97 (m, 6H), 1.82- | |
| | | 1.70 (m, 2H) | |
| 3 | 4-[4-[4-[4-[4-[5-acetyl-3-[7- | δ = 7.62 (d, J = 8.8 Hz, 1H), 7.56-7.51 | 910.1 |
| | (difluoromethyl)-6-(1- | (m, 1H), 7.43-7.38 (m, 1H), 7.19-7.10 | |
| | methylpyrazol-4-yl)-3,4-dihydro- | (m, 3H), 7.07-6.96 (m, 2H), 6.86 (m, | |
| | 2H-quinolin-1-yl]-6,7-dihydro- | 3H), 6.70-6.34 (m, 1H), 4.34-4.19 (m, | |
| | 4H-pyrazolo[4,3-c]pyridin-1-yl]- | 1H), 4.12 (s, 1H), 4.04 (s, 1H), 4.02- | |
| | 1-piperidyl]butoxy]phenyl]-1- | 3.88 (m, 8H), 3.78-3.66 (m, 3H), 3.15- | |
| | piperidyl]-2- | 2.99 (m, 4H), 2.92-2.79 (m, 3H), 2.78- | |
| | (trifluoromethyl)benzonitrile | 2.68 (m, 2H), 2.53-2.37 (m, 2H), 2.33- | |
| | | 2.20 (m, 2H), 2.16 (s, 1H), 2.12-2.03 | |
| | | (m, 5H), 2.02-1.88 (m, 4H), 1.86-1.65 | |
| | | (m, 7H) | |
| 12 | 1-[5-acetyl-1-[1-[1-[4-[1-[4- | (DMSO-d₆): δ = 7.89-7.73 (m, 1H), | 844.4 |
| | cyano-3-(trifluoromethyl) | 7.56-7.22 (m, 8H), 6.47-6.33 (m, 1H), | |
| | phenyl]-4-piperidyl] phenyl] | 4.54-4.45 (m, 2H), 4.41-4.35 (m, 2H), | |
| | piperidine-4-carbonyl]-4- | 4.22-4.15 (m, 3H), 3.79-3.69 (m, 2H), | |
| | piperidyl]-6,7-dihydro-4H- | 3.67-3.54 (m, 4H), 3.25 (s, 3H), 3.12- | |
| | pyrazolo[4,3-c] pyridin-3-yl]- | 2.98 (m, 3H), 2.94-2.69 (m, 6H), 2.08 | |
| | 3,4-dihydro-2H-quinoline-6- | (s, 2H), 2.03-1.80 (m, 12H), 1.77-1.59 | |
| | carbonitrile | (m, 3H) | |
| 13 | 1-[5-acetyl-1-[1-[[1-[4-[1-[4- | (DMSO-d₆) δ = 7.88-7.74 (m, 1H), 7.42- | 830.4 |
| | cyano-3-(trifluoromethyl) | 7.25 (m, 4H), 7.13-7.00 (m, 2H), 6.92- | |
| | phenyl]-4-piperidyl] phenyl]-4- | 6.78 (m, 2H), 6.50-6.37 (m, 1H), 4.21- | |
| | piperidyl] methyl]-4-piperidyl]- | 4.02 (m, 5H), 3.77-3.67 (m, 2H), 3.64- | |
| | 6,7-dihydro-4H-pyrazolo[4,3-c] | 3.56 (m, 4H), 3.09-2.98 (m, 2H), 2.97- | |
| | pyridin-3-yl]-3,4-dihydro-2H- | 2.89 (m, 2H), 2.87-2.78 (m, 3H), 2.76- | |
| | quinoline-6-carbonitrile | 2.65 (m, 2H), 2.63-2.57 (m, 2H), 2.23- | |
| | | 2.13 (m, 2H), 2.08-1.93 (m, 9H), 1.86- | |
| | | 1.74 (m, 6H), 1.66-1.55 (m, 3H), 1.24- | |
| | | 1.16 (m, 2H). | |
| 14 | 4-[4-[4-[4-[2-[5-[8-(5-acetyl-1- | δ = 9.84 (m, 1H), 9.26 (s, 1H), 8.54- | 893.1 |
| | tetrahydropyran-4-yl-6,7- | 8.33 (m, 1H), 8.12 (m, 1H), 7.98-7.70 | |
| | dihydro-4H-pyrazolo[4,3- | (m, 2H), 7.68-7.54 (m, 2H), 7.42-7.30 | |
| | c]pyridin-3-yl)-3-isoquinolyl]-2- | (m, 2H), 7.21-7.08 (m, 3H), 7.05-6.88 | |
| | pyridyl]ethyl]-1- | (m, 2H), 4.67 (s, 1H), 4.51 (s, 1H), 4.38- | |
| | piperidyl]phenyl]-1-piperidyl]-2- | 4.24 (m, 1H), 4.18 (m, 2H), 4.09-3.97 | |
| | (trifluoromethyl)benzonitrile | (m, 3H), 3.91-3.80 (m, 1H), 3.74-3.49 | |
| | | (m, 4H), 3.11-3.02 (m, 2H), 3.02-2.91 | |
| | | (m, 3H), 2.90-2.85 (m, 1H), 2.78-2.64 | |
| | | (m, 2H), 2.55-2.35 (m, 2H), 2.23-2.21 | |
| | | (m, 1H), 2.09 (s, 2H), 2.01-1.95 (m, | |
| | | 4H), 1.95-1.88 (m, 2H), 1.87-1.73 (m, | |
| | | 6H), 1.71-1.64 (m, 2H) | |
| 15 | 4-[4-[4-[4-[2-[4-[8-(5-acetyl-1- | δ = 9.80-9.63 (m, 1H), 8.22-8.07 (m, | 898 |
| | tetrahydropyran-4-yl-6,7- | 2H), 7.87-7.73 (m, 2H), 7.72-7.64 (m, | |
| | dihydro-4H-pyrazolo[4,3- | 1H), 7.61 (m, 1H), 7.54-7.40 (m, 1H), | |
| | c]pyridin-3-yl)-3- | 7.19-7.09 (m, 1H), 7.07-6.92 (m, 3H), | |
| | isoquinolyl]pyrazol-1- | 6.92-6.77 (m, 2H), 4.73-4.55 (m, 1H), | |
| | yl]ethoxy]-1-piperidyl]phenyl]- | 4.51-4.43 (m, 1H), 4.42-4.33 (m, 2H), | |
| | 1-piperidyl]-2- | 4.33-4.21 (m, 1H), 4.20-4.11 (m, 2H), | |
| | (trifluoromethyl)benzonitrile | 4.08-3.78 (m, 7H), 3.61-3.51 (m, 2H), | |
| | | 3.49-3.27 (m, 3H), 3.11-2.97 (m, 2H), | |
| | | 2.95-2.82 (m, 4H), 2.73-2.55 (m, 1H), | |
| | | 2.53-2.34 (m, 2H), 2.21 (s, 1H), 2.07 (s, | |
| | | 1H), 2.01-1.87 (m, 6H), 1.78-1.68 (m, | |
| | | 4H) | |

### Example 2: Synthesis of 4-[3-[4-[4-[3-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]propoxy]phenyl]phenyl]-4,4-dimethyl-5-oxo-2-thioxo-imidazolidin-1-yl]-2-(trifluoromethyl)benzonitrile (Compound 1)

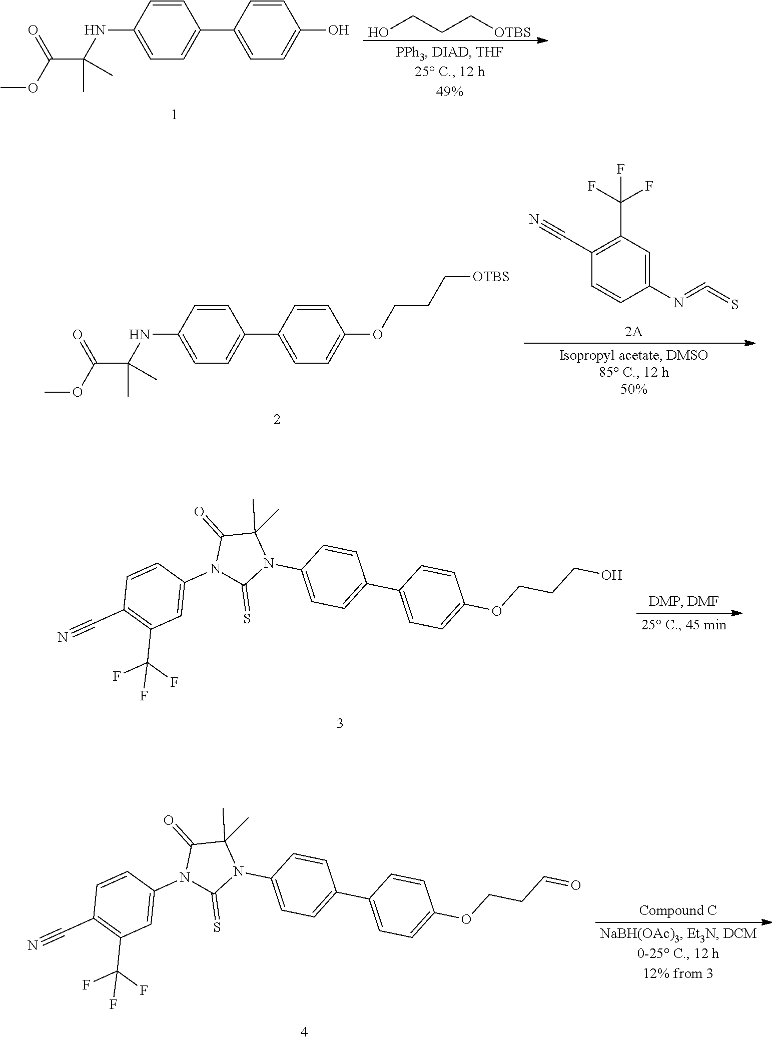 [SMILES: COC(=O)C(C)(C)Nc1ccc(-c2ccc(O)cc2)cc1 ; CC(C)(C)[Si](C)(C)OCCCO ; COC(=O)C(C)(C)Nc1ccc(-c2ccc(OCCCO[Si](C)(C)C(C)(C)C)cc2)cc1 ; N#Cc1ccc(N=C=S)cc1C(F)(F)F ; CC1(C)C(=O)N(c2ccc(C#N)c(C(F)(F)F)c2)C(=S)N1c1ccc(-c2ccc(OCCCO)cc2)cc1 ; CC1(C)C(=O)N(c2ccc(C#N)c(C(F)(F)F)c2)C(=S)N1c1ccc(-c2ccc(OCCC=O)cc2)cc1]

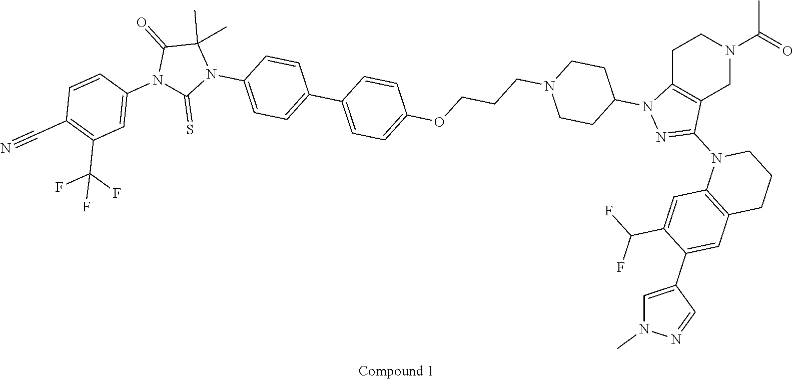 [SMILES: CC(=O)N1CCc2c(c(N3CCCc4cc(-c5cnn(C)c5)c(C(F)F)cc43)nn2C2CCN(CCCOc3ccc(-c4ccc(N5C(=S)N(c6ccc(C#N)c(C(F)(F)F)c6)C(=O)C5(C)C)cc4)cc3)CC2)C1]

Synthesis of 1 was reported in Archiv der Pharmazie, 2022, vol. 355, #5, art. no. 2100467

Synthesis of 2A was reported in ACS Medicinal Chemistry Letters, 2021, vol. 12, #8, p. 1245-1252.

To a solution of methyl 2-[4-(4-hydroxyphenyl)anilino]-2-methyl-propanoate (2.00 g, 7.01 mmol, 1.0 eq), PPh₃ (0.3 M, 35.1 mL, 1.5 eq) and 3-[tert-butyl(dimethyl)silyl]oxypropan-1-ol (2.00 g, 10.5 mmol, 1.5 eq) in Toluene (40 mL) was added DEAD (1.34 g, 7.71 mmol, 1.40 mL, 1.1 eq) and stirred at 25° C. for 12 h under N₂. The mixture was poured into ice-water (120 mL). The aqueous phase was extracted with ethyl acetate (40 mL×3). The combined organic phase was washed with brine (30 mL×2), dried with anhydrous sodium sulfate, filtered and concentrated under vacuum. The residue was purified by semi-preparative reverse phase HPLC (column: Phenomenex luna C18 150×40 mm×15 um; mobile phase: [water (FA)-ACN]; gradient: 90%-100% B over 15 min). Compound methyl 2-[4-[4-[3-[tert-butyl(dimethyl)silyl]oxypropoxy]phenyl]anilino]-2-methyl-propanoate (1.70 g, 3.42 mmol, 49% yield) was obtained as a light yellow solid.

LC-MS: MS (ESI⁺): t_{R}=0.749 min, m/z=458.4 [M+H⁺]

To a solution of methyl 2-[4-[4-[3-[tert-butyl(dimethyl)silyl]oxypropoxy]phenyl]anilino]-2-methyl-propanoate (1.00 g, 2.18 mmol, 1.0 eq) in DMSO (8 mL) and Isopropyl acetate (8 mL) was added 4-isothiocyanato-2-(trifluoromethyl)benzonitrile (760 mg, 3.33 mmol, 1.5 eq) and stirred at 85° C. for 12 h under N₂. The mixture was poured into ice-water (40 mL). The aqueous phase was extracted with ethyl acetate (25 mL×3). The combined organic phase was washed with brine (30 mL×2), dried with anhydrous sodium sulfate, filtered and concentrated under vacuum. The residue was purified by semi-preparative reverse phase HPLC (column: Phenomenex luna C18 (250×70 mm, 10 um); mobile phase: [water (FA)-ACN]; gradient: 80%-100% B over 22 min). Compound 4-[3-[4-[4-(3-hydroxypropoxy)phenyl]phenyl]-4,4-dimethyl-5-oxo-2-thioxo-imidazolidin-1-yl]-2-(trifluoromethyl)benzonitrile (0.60 g, 1.10 mmol, 50% yield) was obtained as a off-white solid.

LC-MS: MS (ESI⁺): t_{R}=0.638 min, m/z=540.3 [M+H⁺]

To a solution of 4-[3-[4-[4-(3-hydroxypropoxy)phenyl]phenyl]-4,4-dimethyl-5-oxo-2-thioxo-imidazolidin-1-yl]-2-(trifluoromethyl)benzonitrile (0.12 g, 222 μmol, 1.0 eq) in DMF (1 mL) was added DMP (236 mg, 556 μmol, 172 μL, 2.5 eq) and stirred at 25° C. for 45 min. This mixture which contains compound 4-[4,4-dimethyl-5-oxo-3-[4-[4-(3-oxopropoxy)phenyl]phenyl]-2-thioxo-imidazolidin-1-yl]-2-(trifluoromethyl)benzonitrile was directly used in the next step.

The above-mentioned mixture containing 4 was added to a solution of 1-[3-[7-(difluoromethyl)-6-(1-methylpyrazol-4-yl)-3,4-dihydro-2H-quinolin-1-yl]-1-(4-piperidyl)-6,7-dihydro-4H-pyrazolo[4,3-c]pyridin-5-yl]ethanone (80 mg, 157 μmol, 1.0 eq), Et₃N (159 mg, 1.57 mmol, 219 μL, 10.0 eq) and NaBH(OAc)₃ (333 mg, 1.57 mmol, 10.0 eq) in DCM (10 mL) at 0° C. and stirred at 25° C. for 12 h under N₂. The mixture was diluted with ethyl acetate (100 mL), washed with water (15 mL×2), brine (15 mL), dried with anhydrous sodium sulfate, filtered and concentrated under vacuum. The residue was purified by semi-preparative reverse phase HPLC (column: Phenomenex luna C18 150×25 mm×10 um; mobile phase: [water (FA)-ACN]; gradient: 34%-64% B over 10 min). Compound 4-[3-[4-[4-[3-[4-[5-acetyl-3-[7-(difluoromethyl)-6-(1-methylpyrazol-4-yl)-3,4-dihydro-2H-quinolin-1-yl]-6,7-dihydro-4H-pyrazolo[4,3-c]pyridin-1-yl]-1-piperidyl]propoxy]phenyl]phenyl]-4,4-dimethyl-5-oxo-2-thioxo-imidazolidin-1-yl]-2-(trifluoromethyl)benzonitrile (19.51 mg, 18.3 μmol, 12% yield over two steps) was obtained as a yellow solid.

1H NMR (400 MHz, DMSO-d₆): δ=8.40 (d, J=8.4 Hz, 1H) 8.32 (s, 1H) 8.06-8.14 (m, 1H) 7.80 (m, 2H) 7.75 (s, 1H) 7.68 (m, 2H) 7.49 (s, 1H) 7.42 (d, J=8.4 Hz, 2H) 7.00-7.13 (m, 3H) 6.63-6.95 (m, 2H) 3.97-4.19 (m, 5H) 3.86 (s, 3H) 3.65-3.78 (m, 2H) 3.55-3.63 (m, 2H) 3.39-3.46 (m, 1H) 2.95-3.04 (m, 2H) 2.72-2.88 (m, 4H) 2.03-2.13 (m, 5H) 1.80-2.02 (m, 9H) 1.54 (s, 6H).

LC-MS: MS (ESI⁺): t_{R}=2.136 min, m/z=1031.6 [M+H⁺]

### Example 3: Synthesis of 3-[7-(difluoromethyl)-6-(1-methylpyrazol-4-yl)-3,4-dihydro-2H-quinolin-1-yl]-1-[1-[2-[2-[[1-[[1-[4-(6-hydroxy-2-phenyl-tetralin-1-yl)phenyl]-4-piperidyl]methyl]-4-piperidyl]methoxy]ethoxy]acetyl]-4-piperidyl]-N-methyl-6,7-dihydro-4H-pyrazolo[4,3-c]pyridine-5-carboxamide (Compound 10)

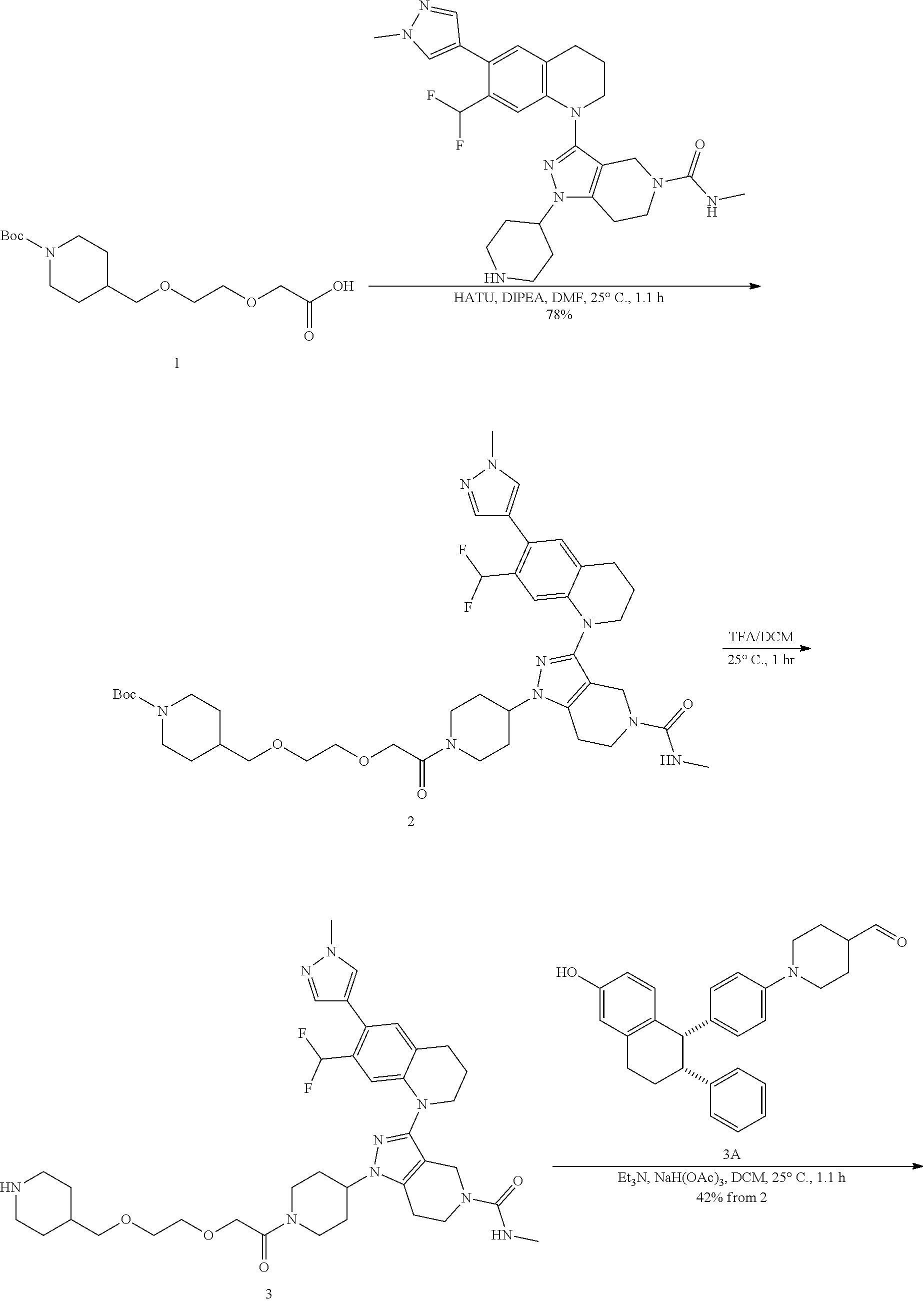

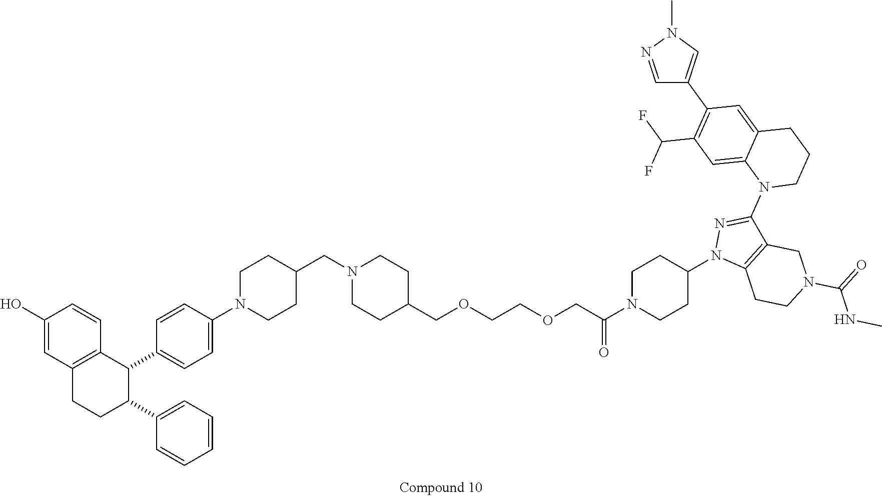 [SMILES: CNC(=O)N1CCc2c(c(N3CCCc4cc(-c5cnn(C)c5)c(C(F)F)cc43)nn2C2CCN(C(=O)COCCOCC3CCN(CC4CCN(c5ccc([C@H]6c7ccc(O)cc7CC[C@H]6c6ccccc6)cc5)CC4)CC3)CC2)C1]

Synthesis of 1 was reported in WO2021/188948, 2021, A1.

Synthesis of 3A was reported in US2021/60008, 2021, A1.

To a solution of 2-[2-[(1-tert-butoxycarbonyl-4-piperidyl)methoxy]ethoxy]acetic acid (450 mg, 1.42 mmol, 1.2 eq), 3-[7-(difluoromethyl)-6-(1-methylpyrazol-4-yl)-3,4-dihydro-2H-quinolin-1-yl]-N-methyl-1-(4-piperidyl)-6,7-dihydro-4H-pyrazolo[4,3-c]pyridine-5-carboxamide (600 mg, 1.14 mmol, 1.0 eq) in DMF (4 mL) was added DIPEA (519 mg, 4.02 mmol, 700 μL, 3.5 eq) and stirred at 25° C. for 0.1 h. The mixture was added HATU (524 mg, 1.38 mmol, 1.2 eq) and stirred at 25° C. for 1 h. The residue was diluted with water 100 mL and extracted with Ethyl acetate (100 mL×3). The combined organic layers were washed with brine (100 mL×3), dried over Na₂SO₄, filtered and concentrated under reduced pressure to give a residue. The residue was purified by prep-TLC (SiO₂, DCM/MeOH=100/1 to 30/1). Compound tert-butyl 4-[2-[2-[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]-2-oxo-ethoxy]ethoxymethyl]piperidine-1-carboxylate (738 mg, 896 μmol, 78% yield) was obtained as a white solid.

LC-MS: MS (ESI⁺): t_{R}=0.588 min, m/z=824.7 [M+H⁺]

To a solution of tert-butyl 4-[2-[2-[4-[3-[7-(difluoromethyl)-6-(1-methylpyrazol-4-yl)-3,4-dihydro-2H-quinolin-1-yl]-5-(methylcarbamoyl)-6,7-dihydro-4H-pyrazolo[4,3-c]pyridin-1-yl]-1-piperidyl]-2-oxo-ethoxy]ethoxymethyl]piperidine-1-carboxylate (1.20 g, 1.46 mmol, 1.0 eq) in DCM (3.0 mL) was added TFA (2.5 mL). The mixture was stirred at 25° C. for 1 h. The reaction mixture was concentrated under reduced pressure to give a residue. Compound 3-[7-(difluoromethyl)-6-(1-methylpyrazol-4-yl)-3,4-dihydro-2H-quinolin-1-yl]-N-methyl-1-[1-[2-[2-(4-piperidylmethoxy)ethoxy]acetyl]-4-piperidyl]-6,7-dihydro-4H-pyrazolo[4,3-c]pyridine-5-carboxamide (1.2 g) was obtained as a yellow solid and directly used in the next step without further purification.

LC-MS: MS (ESI⁺): t_{R}=0.493 min, m/z=724.7 [M+H⁺]

To a solution of 3-[7-(difluoromethyl)-6-(1-methylpyrazol-4-yl)-3,4-dihydro-2H-quinolin-1-yl]-N-methyl-1-[1-[2-[2-(4-piperidylmethoxy)ethoxy]acetyl]-4-piperidyl]-6,7-dihydro-4H-pyrazolo[4,3-c]pyridine-5-carboxamide (1.2 g), 1-[4-(6-hydroxy-2-phenyl-tetralin-1-yl)phenyl]piperidine-4-carbaldehyde (530 mg, 1.29 mmol, 1.0 eq) in DCM (10 mL) was added Et₃N (722 mg, 7.14 mmol, 994 μL, 5.8 eq) and stirred at 25° C. for 0.1 h. The mixture was added NaBH(OAc)₃ (845 mg, 3.99 mmol, 3.2 eq) and stirred at 25° C. for 1 h. The residue was diluted with water 100 mL and extracted with Ethyl acetate (100 mL×3). The combined organic layers were washed with brine (100 mL×3), dried over Na₂SO₄, filtered and concentrated under reduced pressure to give a residue. The residue was purified by column chromatography (SiO₂, DCM/MeOH=100/1 to 10/1). Compound 3-[7-(difluoromethyl)-6-(1-methylpyrazol-4-yl)-3,4-dihydro-2H-quinolin-1-yl]-1-[1-[2-[2-[[1-[[1-[4-(6-hydroxy-2-phenyl-tetralin-1-yl)phenyl]-4-piperidyl]methyl]-4-piperidyl]methoxy]ethoxy]acetyl]-4-piperidyl]-N-methyl-6,7-dihydro-4H-pyrazolo[4,3-c]pyridine-5-carboxamide (598.85 mg, 513.58 μmol, 42% yield over two steps) was obtained as an off-white solid.

¹H NMR (400 MHz, CHLOROFORM-d) 6=7.53 (s, 1H), 7.41 (s, 1H), 7.20-7.10 (m, 3H), 7.04 (s, 1H), 6.88-6.66 (m, 5H), 6.62-6.38 (m, 4H), 6.27 (br d, J=8.4 Hz, 2H), 4.71-4.61 (m, 1H), 4.47 (br d, J=4.8 Hz, 1H), 4.29-4.20 (m, 2H), 4.19-4.04 (m, 3H), 4.02-3.90 (m, 5H), 3.78 (t, 2H), 3.69 (br d, J=5.2 Hz, 4H), 3.62 (br s, 2H), 3.56-3.46 (m, 2H), 3.40-3.29 (m, 3H), 3.17 (t, 1H), 3.09-2.94 (m, 2H), 2.86 (t, 3H), 2.79 (br d, J=4.4 Hz, 3H), 2.76-2.76 (m, 1H), 2.77-2.69 (m, 1H), 2.54 (t 5H), 2.12 (br s, 1H), 2.09-1.89 (m, 6H), 1.84-1.80 (m, 8H), 1.38-1.28 (m, 2H).

LC-MS: MS (ESI⁺): t_{R}=1.567 min, m/z=1119.9 [M+H⁺]

### Example A-1: Oral Solution

To prepare a pharmaceutical composition for oral delivery, a sufficient amount of a compound of Formula (I) or (II), or a pharmaceutically acceptable salt thereof, is added to water (with optional solubilizer(s), optional buffer(s) and taste masking excipients) to provide a 20 mg/mL solution.

### Example A-2: Oral Tablet

A tablet is prepared by mixing 20-50% by weight of a compound of Formula (I) or (II), or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-3: Oral Capsule

To prepare a pharmaceutical composition for oral delivery, 10-500 mg of a compound of Formula (I) or (II), or a pharmaceutically acceptable salt thereof, is mixed with starch or other suitable powder blend. The mixture is incorporated into an oral dosage unit such as a hard gelatin capsule, which is suitable for oral administration.

In another embodiment, 10-500 mg of a compound of Formula (I) or (II), or a pharmaceutically acceptable salt thereof, is placed into Size 4 capsule, or size 1 capsule (hypromellose or hard gelatin) and the capsule is closed.

### Example B-1: Ternary Complex Formation Assay

### Preparation of Mouse Anti-AR Antibody Coated 96 Well Plates

Black MaxiSorp plates: coat with 50 μL of mouse 1/50 diluted AR antibody (Monoclonal Antibody (AR 441 from Thermo Fisher); diluted in phosphate buffered saline (PBS). Spin the liquid to the bottom of the wells, if necessary. Incubate overnight at 4° C. Next day, wash 3 times with 200 μL of TBST. Block with 200 μL of blocking buffer (5% BSA in TBST) at room temperature for 2 hours (BSA: A4503 or A2153 from Sigma). Wash plates 3 times with TBST. Plates are ready for lysates

### Preparation of Dimerizer Treated Lysates

Culture cells in 96 wells at 50K VCaP cells/well for at least 3 days. Plate in 5% Omega charcoal stripped FBS in total volume of 200 uL. Treat with bifunctional for 1.5 hours by adding 1 μL of 200× stock in DMSO. Aspirate media/compound. Add and pipet-to-mix 70 μL of 1× AlphaLisa Lysis Buffer to each well (Perkin Elmer, AlphaLISA SureFire Ultra, diluted in water). Pipet up and down when adding lysis buffer to disrupt cells. Let lysis proceed for 30 min at 4° C.

### Sandwich ELISA

Transfer 50 uL of lysate to MaxiSorp plates coated with antibody and subsequently blocked with BSA. Incubate for 1 hour at room temperature. Flick out the lysates. Wash 3 times with TBST. Add 75 uL of rabbit CBP or p300 antibody to each well (diluted 1:5000 in blocking buffer (5% BSA in TBST). CBP antibody: (D6C5) Rabbit mAb #7389; p300 antibody: (D8Z4E) Rabbit mAb #86377. Incubate overnight at 4° C. Wash 3 times with 300 uL TBST, each time on shaker for 5-10 min. Prepare goat anti-rabbit antibody conjugated to HRP (1/100K dilution in blocking buffer) Invitrogen Catalog number: 32260. Add 75 uL of HRP-conjugated goat anti-rabbit antibody. Incubate at room temperature for 1 hour. Flick out the HRP-antibody mixture. Wash 7-8 times with TBST, each time on shaker for 10 min. Later washes try to overflow the wells so that no residual HRP remains bound to the sides of the well. Prepare and add 75 uL/well of QuantaRed Enhance Chemifluorescent HRP Substrate solution. Immediately read fluorescence on BMG ClarioStar (ex: 570/em 585), and repeat the read in 5 minutes and 15 minutes.

Results of the ternary complex formation assay are shown in Table 2.

TABLE 2

| | Average Ternary |
|---|---|
| Compound | Complex* (nM) |
| 1 | B |
| 2 | D |
| 3 | D |
| 4 | D |
| 12 | D |
| 13 | D |
| 14 | D |
| 15 | D |
| 18 | B |
| 19 | B |
| 20 | B |
| 21 | B |

*A is <1 nM; B is >1 nM and ≤10 nM; C is >10 nM and ≤100 nM; D is active and >100 nM

### Example B-2: VCaP Growth Inhibition Assay

VCaP cells (ATCC Cat #CRL-2876) are plated at 1×10⁴ cells per well of a white plastic 96-well cluster plate (Thermo Scientific Nunc Cat #165306) in 100 uL phenol red-free DMLEM (Gibco Cat #21063-029) media with 500 CSS (Omega Scientific Cat #FB-04). Cluster plates are returned to the incubator (37*C/5% CO2) for 72 hours prior to treatment. Just prior to treatment, 100 uL of phenol red-free DMEM/5% CSS and 60 pM Ri881 (Sigma Cat #R0908-10 mg) was added to each well of the cluster plates (Final R1881 concentration=30 pM). Compounds (200×) were added to each well in a volume of 1 uL, with serial dilutions (1:5) from 20 uM to 2 pM (final concentrations in well are 100 nM to 10 fMV). Cluster plates are returned to the incubator for 9 days.

After the 9-day treatment period, media was removed from the cluster plates by inversion and 100 uL room temperature CTG reagent (Promega Cat #G7573) is added to each well. The cluster plates are placed on a shaker and incubated at room temperature for 20 minutes. Following this shaking/incubation, cluster plates are read on a luminometer (BMG ClarioStar). IC₅₀ values are obtained by graphing the RLU data vs. compound concentration with nonlinear regression 4-parameter logistic curves (GraphPad Prism).

Results of the VCaP growth inhibition assay are shown in Table 3.

TABLE 3

| Compound | IC₅₀** (M) |
|---|---|
| 1 | ++++ |
| 4 | ++++ |
| 6 | +++ |
| 7 | +++ |
| 8 | +++ |
| 9 | +++ |
| 10 | +++ |
| 11 | ++++ |
| 12 | ++++ |
| 13 | ++++ |
| 14 | ++++ |
| 15 | +++ |

**++++ is IC₅₀ active and >1E−7; +++ is 1E−7 ≤ IC₅₀ > 1E−9; ++ is 1E−9 ≤ IC₅₀ > 1E−10; + is IC₅₀ ≤ 1E−10

### Example B-4: HEK-293 Growth Inhibition Assay

HEK-293 cells (ATCC Cat #CRL-1573) are plated at 3×10³ cells per well of a white plastic 96-well cluster plate (Thermo Scientific Nunc Cat #165306) in 200 uL DMLEM (ATCC Cat #30-2002) media with 10% FBS (Gibco Cat #26140-079). Cluster plates are returned to the incubator (37*C/5% CO2) for 24 hours prior to treatment. Compounds (200×) were added to each well in a volume of 1 uL, with serial dilutions (1:5) from 2 mM to 200 pM (final concentrations in well are 10 uM to 1 pM). Cluster plates are returned to the incubator for 3 days.

After the 3-day treatment period, 100 uL media was removed from each well by aspiration and 100 uL room temperature CTG reagent (Promega Cat #G7573) is added back to each well. The cluster plates are placed on a shaker and incubated at room temperature for 20 minutes. Following this shaking/incubation, cluster plates are read on a luminometer (BMG ClarioStar). ICs₅₀ values are obtained by graphing the RLU data vs. compound concentration with nonlinear regression 4-parameter logistic curves (GraphPad Prism).

Results of the HEK-293 growth inhibition assay are shown in Table 4.

TABLE 4

| Compound | IC₅₀ (μM) |
|---|---|
| 10 | >1 |

What is claimed is:

1. A heterobifunctional conditional inhibitor compound of Formula (I): 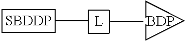
wherein:
SBDDP is a silent binder of a disease-dependent protein (DDP);
L is an optional linker; and
BDP is a binder of a disease protein (DP);
wherein DDP and DP are both expressed in a cell of interest (COI) and the relative abundance of the DP in the COI is greater than the relative abundance of the DDP in the COI; or
wherein the COI is a diseased cell, and the DP is overexpressed, overactive, or both overexpressed and overactive, or amplified in the diseased cell as compared to when the COI is a non-diseased cell.

2. The compound of claim 1, wherein the activity of the DDP is reduced or inhibited by the compound of Formula (I) when the DDP and DP are both expressed in the same COI and the relative abundance of the DP in the COI is greater than the relative abundance of the DDP in the COI.

3. The compound of claim 1 or 2, wherein DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3.

4. The compound of claim 1 or 2, wherein DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

5. The compound of claim 1 or 2, wherein DDP is a human bromodomain-containing protein (BRD).

6. The compound of claim 1 or 2, wherein DDP is a human bromodomain-containing protein that is a Group Ia BRD, Group Ib BRD, Group II BRD, Group IIIa BRD, Group IIIb BRD, Group IIII BRD, Group IV BRD, Group V BRD, Group VI BRD, Group VI BRD, Group VIII BRD, or group IX BRD.

7. The compound of claim 6, wherein:
the Group Ia BRD is selected from PCAF, GCN5L2, p300/CBP, TAF1 and TAF1L;
the Group Ib BRD is selected from BRPF1A/B (BR140), BRPF2 (BRD1) BRPF3 and BRD8 (SMAP);
the Group II BRD is selected from histone methyltransferases ASH1L and MLL;
the Group IIIa BRD is selected from chromatin remodeling factors SMARCA2 (BRM), SMARCA4 (BRG1), BRD7, BRD9 and PBRM1 (polybromo);
the Group IIIb BRD is selected from ISWI family chromatin remodeling factors BAZ1A (ACF1), BAZ1B (WSTF, William-Beuren syndrome transcription factor), BAZ2A, BAZ2B, BPTF and CECR2;
the Group IV BRD is selected from ATPase family AAA domain-containing proteins ATAD2 and ATAD2B;
the Group V BRD is selected from BET family proteins BRD2, BRD3, BRD4 and testis-specific BRDT;
the Group VI BRD is selected from Tripartite-motif-containing (TRIM) family proteins TRIM24 (TIF1α), TRIM28 (TIF1β, KAP1), TRIM33 (TIF1γ) and TRIM66 (TIF1δ);
the Group VIII BRD is selected from speckled protein (SP) family proteins SP100, SP110, SP140 and SP140L;
the Group VIII BRD is selected from transcriptional corepressors ZMYND8 and ZMYDN11; and
the Group IX is selected from WD-repeat proteins BRWD1 (WDR9), BRWD3 and PHIP (WDR11).

8. The compound of claim 1 or 2, wherein DDP is a human bromodomain-containing protein (BRD) is:
a Group Ia BRD selected from PCAF, GCN5L2, p300/CBP, TAF1 and TAF1L;
a Group Ib BRD selected from BRPF1A/B (BR140), BRPF2 (BRD1) BRPF3 and BRD8 (SMAP);
a Group II BRD selected from histone methyltransferases ASH1L and MLL;
a Group IIIa BRD selected from chromatin remodeling factors SMARCA2 (BRM), SMARCA4 (BRG1), BRD7, BRD9 and PBRM1 (polybromo);
a the Group IIIb BRD selected from ISWI family chromatin remodeling factors BAZ1A (ACF1), BAZ1B (WSTF, William-Beuren syndrome transcription factor), BAZ2A, BAZ2B, BPTF and CECR2;
a Group IV BRD selected from ATPase family AAA domain-containing proteins ATAD2 and ATAD2B;
a Group VI BRD selected from Tripartite-motif-containing (TRIM) family proteins TRIM24 (TIF1α), TRIM28 (TIF1β, KAP1), TRIM33 (TIF1γ) and TRIM66 (TIF1δ);
a Group VIII BRD selected from speckled protein (SP) family proteins SP100, SP110, SP140 and SP140L;
a Group VIII BRD selected from transcriptional corepressors ZMYND8 and ZMYDN11; and
a Group IX selected from WD-repeat proteins BRWD1 (WDR9), BRWD3 and PHIP (WDR11).

9. A heterobifunctional compound of Formula (Ia), comprising: 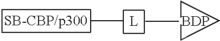
SB-CBP/p300 a silent binder of human CREB-binding protein (CBP) or human E1A-binding protein p300 (p300) (CBP/p300);
BDP is a binder of a disease protein (DP);
L is an optional linker covalently connecting SB-CBP/p300 to BDP;
wherein L is covalently attached at a position of SB-CBP/p300 that is solvent exposed when SB-CBP/p300 binds to CBP/p300, and L is covalently attached at a position of BDP that is solvent exposed when BDP binds to DP;
wherein CBP/p300 and DP are both expressed in a cell of interest (COI) and the relative abundance of the DP in the COI is greater than the relative abundance of CBP/p300 in the COI; or
wherein the COI is a diseased cell, and the DP is overexpressed, overactive, or both overexpressed and overactive, or amplified in the diseased cell as compared to when the COI is a non-diseased cell.

10. The compound of any one of claims 1-9, wherein the DP is androgen receptor (AR), estrogen receptor (ER), progesterone receptor (PR), glucocorticoid receptor (GR), mineralocorticoid receptor (MR), retinoic acid receptor-related orphan nuclear receptor γ (RORγ), angiotensin receptor, apelin receptor, bombesin receptor, bradykinin receptor, calcitonin receptor, chemokine receptor, cholecytokinin receptor, chymase, corticotropic-releasing factor receptor, galanin receptor, ghrelin receptor, glucagon receptor, glycoprotein hormone receptor, gonadotropin-releasing hormone receptor, indoleamine 2,3-dioxygenase 1 (IDO1), kisspeptin receptor, melanocortin receptor, motilin receptor, neuromedin U receptor, neuropeptide FF/AF receptor, neuropeptide S receptor, neuropeptide W/B receptor, neuropeptide Y receptor, opioid receptor, orexin receptor, parathyroid hormone receptor, prokineticin receptor, prolactin-releasing peptide receptor, QRFP receptor, relaxin family peptide receptor, somatostatin receptor, tachykinin receptor, thyrotropin-releasing hormone receptor, urotensin receptor, vasopressin, oxytocin receptor, vasoactive intestinal peptide receptor, PACAP receptor, HER1, HER2, HER3, HER4, KRAS, KRAS-G12C, HRAS, NRAS, or BCL6.

11. The compound of any one of claim 9 or 10, wherein SB-CBP/p300 binds to the bromodomain of human CREB-binding protein (CBP) or human E1A-binding protein p300 (p300) (CBP/p300).

12. The compound of claim 11, or a pharmaceutically acceptable salt or solvate thereof, wherein SB-CBP/p300 binds in the acetyl-lysine (KAc) binding site of the bromodomain of human CREB-binding protein (CBP) or human E1A-binding protein p300 (p300) (CBP/p300).

13. The compound of claim 12, or a pharmaceutically acceptable salt or solvate thereof, wherein:
the moiety comprising an acetyl-lysine mimetic makes or mimics a hydrogen bond interaction to Asn1168 in the Asn-binding pocket of the bromodomain of CBP, or makes or mimics a hydrogen bond interaction to Asn1132 in the Asn-binding pocket of the bromodomain of p300.

14. The compound of any one of claims 12-13, or a pharmaceutically acceptable salt or solvate thereof, wherein: SBDDP further comprises a moiety that interacts with Arg1173 in the bromodomain of CBP or Asn1137 in the bromodomain of p300.

15. The compound of any one of claims 12-14, or a pharmaceutically acceptable salt or solvate thereof, wherein the moiety comprising an acetyl-lysine mimetic comprises a moiety selected from pyrrolidonyl, phenyl, pyridinyl, pyridinonyl, triazolyl, pyrrolyl, isoxazolyl, pyrazolyl, quinolinyl, tetrahydroquinolinyl, isoquinolinyl, tetrahydroisoquinolinyl, quinazolonyl, quinazolinyl, dihydroquinazolinonyl, imidazo[4,5-c]quinolinyl fused to a dimethylisoxazolyl, triazolophthalazinyl, indolizinyl, benzoimidazolyl, isoxazole-indolizinyl, thienodiazepine-indolizinyl, benzodiazepine-indolizinyl, 5-isoxazolylbenzimidazolyl, 6-isoxazolylbenzimidazolyl, 7-isoxazolo-quinolinyl, diazobenzyl, triazolophthalazinyl, isoxazoloquinolinyl, 2-thiazolidinonyl, triazolopyrimidinyl, thienodiazepinyl, benzodiazepinyl, benzotriazepinyl, triazolobenzodiazepinyl, triazolothienodiazepinyl, triazolothienodiazepinyl, and isoxazole-azepinyl.

16. The compound of any one of claims 12-15, or a pharmaceutically acceptable salt or solvate thereof, wherein SB-CBP/p300 further comprises:
1) a moiety that occupies the lipophilic shelf (LPF) region of the bromodomain of CBP/p300; or
2) a moiety that occupies the BC Loop region of the bromodomain of CBP/p300; or
3) both 1) and 2);
wherein L is covalently attached to SB-CBP/p300 on:
the acetyl-lysine mimetic moiety; or
the moiety that occupies the lipophilic shelf (LPF) region of the bromodomain of CBP/p300, if present; or
the moiety that occupies the BC Loop region of the bromodomain of CBP/p300, if present;
wherein L is covalently attached to SB-CBP/p300 at a position that does not interfere with the binding of the acetyl-lysine mimetic moiety in the acetylated lysine (KAc) binding site of CBP/p300.

17. The compound of any one of claims 12-16, or a pharmaceutically acceptable salt or solvate thereof, wherein the moiety comprising an acetyl-lysine mimetic comprises a moiety selected from: 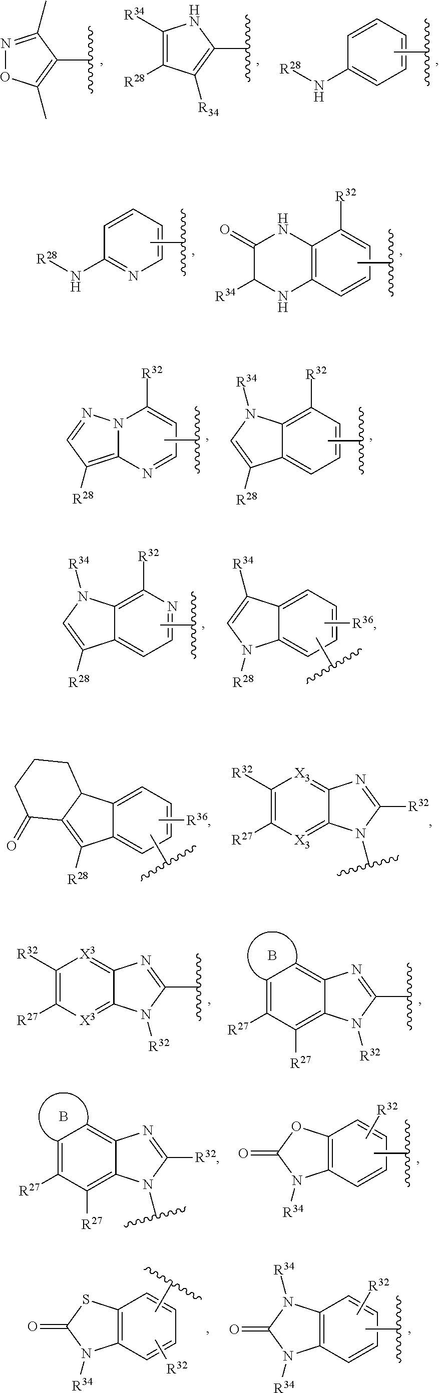 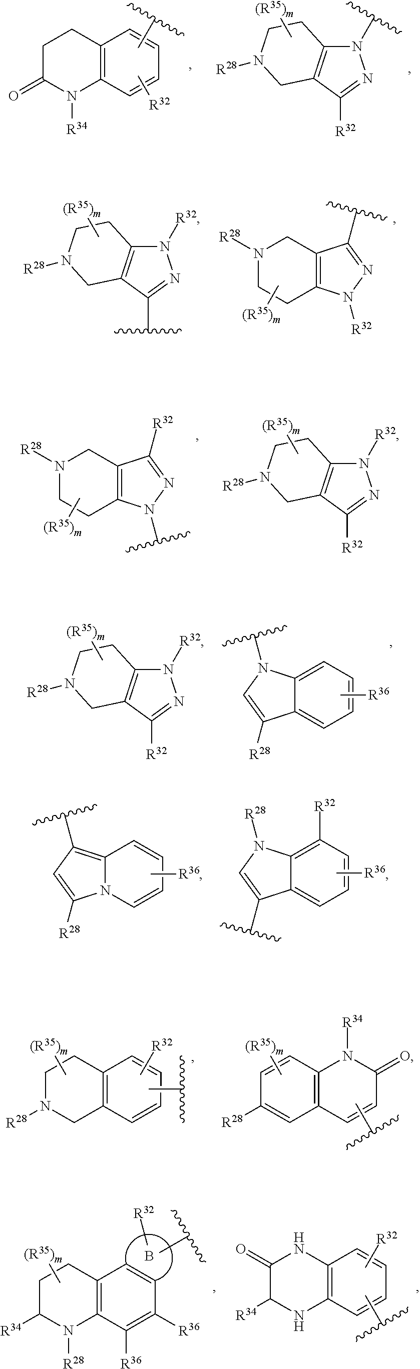 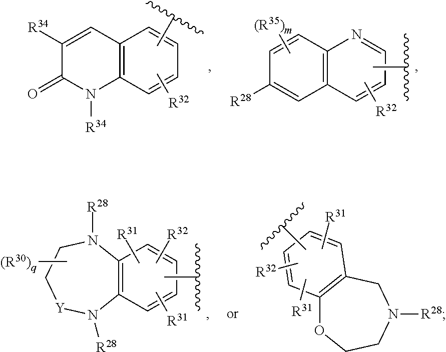
each R³² is independently an optional moiety that occupies the lipophilic shelf (LPF) region of the bromodomain of CBP/p300;
or each R³² is independently an optional moiety that occupies the BC Loop region of the bromodomain of CBP/p300; [symbol]
is the point of attachment to L that covalently connects SB-CBP/p300 to BDP;
or R³² comprises [symbol] and L that covalently connects SB-CBP/p300 to BDP is attached to R³²;
each R²⁸ is independently hydrogen, substituted or unsubstituted C₁-C₆alkyl, substituted or unsubstituted C₁-C₆fluoroalkyl, —C(═O)R^{b}, or —C(═O)N(R^{b})₂;
each R³⁴ is independently hydrogen or substituted or unsubstituted C₁-C₆alkyl;
each R³⁵ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl, —CN, —OH, —OR^{a}, or —N(R^{b})₂;
m is 0, 1, 2, 3, or 4;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
Y is —C(R³⁰)₂— or C(═O);
each X³ is independently CR²⁷ or N;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
each R³¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)NR^{b}, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
each R³⁶ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR^{a}, or —N(R^{b})₂;
Ring B is a fused substituted or unsubstituted 5 or 6 membered heterocycloalkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

18. The compound of claim 12-17, or a pharmaceutically acceptable salt or solvate thereof, wherein the moiety that occupies the lipophilic shelf (LPF) region of the bromodomain of CBP/p300 is R³², wherein:
R³² is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₂cycloalkyl, substituted or unsubstituted 3- to 12-membered heterocycloalkyl, substituted or unsubstituted aryl, or substituted or unsubstituted heteroaryl;
or R³² is 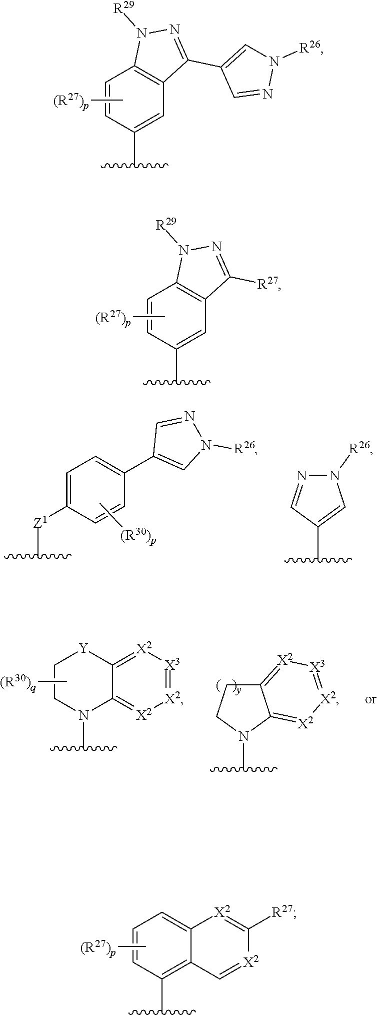
y is 1 or 2;
Z¹ is —NR^{c}—, —O—, or —S—;
R^{c} is hydrogen or substituted or unsubstituted C₁-C₆alkyl;
R²⁶ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each X² is independently —CR³⁰— or —N—;
each X³ is independently —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen or substituted or unsubstituted C₁-C₆alkyl;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
Y is —C(R³⁰)₂— or N(R²⁸)—;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
or R³² is -L-C;
L is substituted or unsubstituted C₁-C₆alkyl or substituted or unsubstituted C₁-C₆heteroalkyl;
C is substituted or unsubstituted C₃-C₈cycloalkyl, substituted or unsubstituted 3- to 8-membered heterocycloalkyl, substituted or unsubstituted aryl, or substituted or unsubstituted heteroaryl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

19. The compound of any one of claims 12-18, or a pharmaceutically acceptable salt or solvate thereof, wherein the moiety that occupies the BC Loop region of the bromodomain of CBP/p300 is R³², wherein: 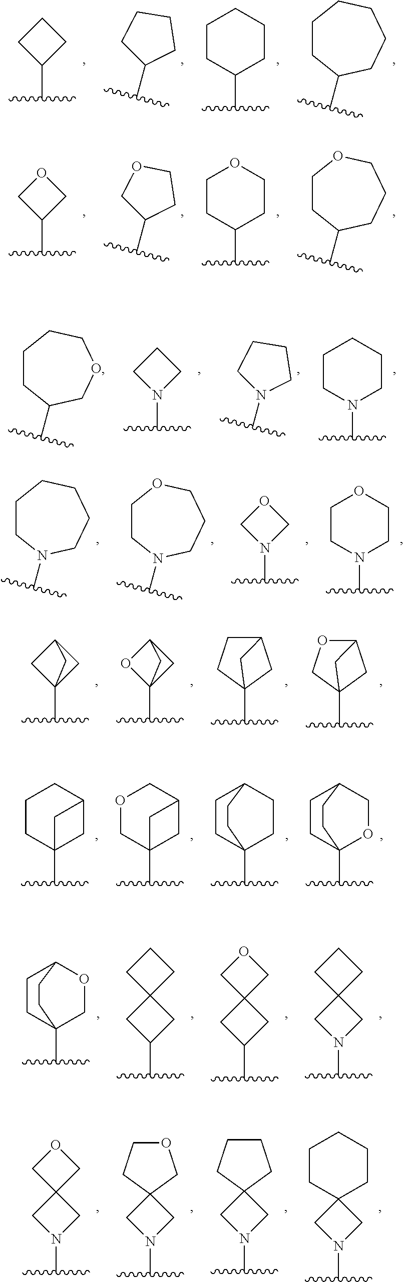 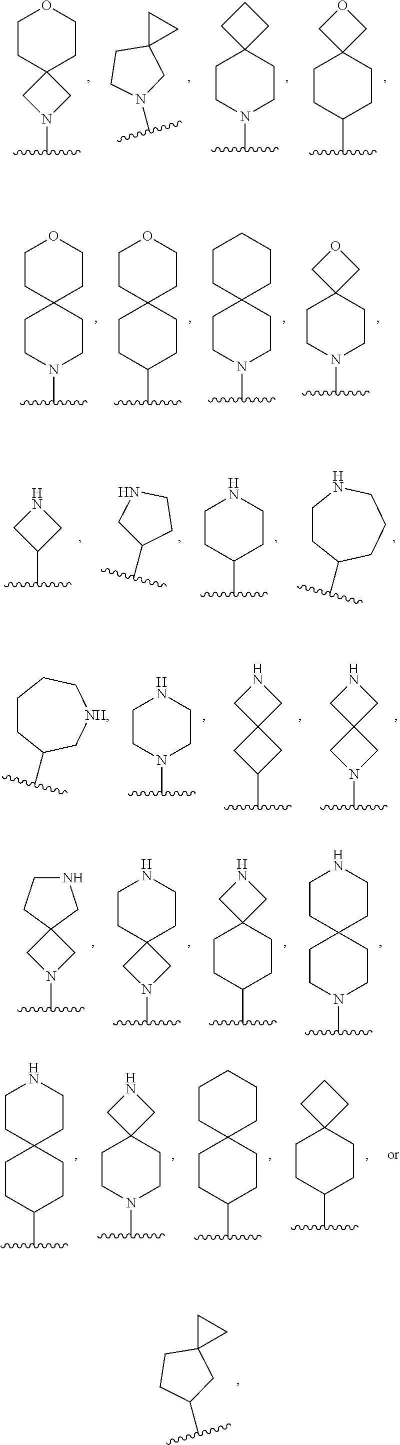
each of which is substituted or unsubstituted.

20. The compound of any one of claims 12-19, or a pharmaceutically acceptable salt or solvate thereof, wherein the moiety comprising an acetyl-lysine mimetic comprises

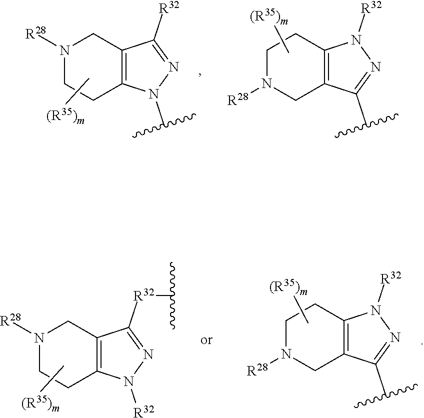

21. The compound of any one of claims 12-19, or a pharmaceutically acceptable salt or solvate thereof, wherein the moiety comprising an acetyl-lysine mimetic comprises

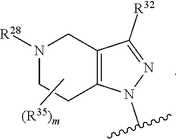

22. The compound of claim 21, or a pharmaceutically acceptable salt or solvate thereof, wherein:
R³² is 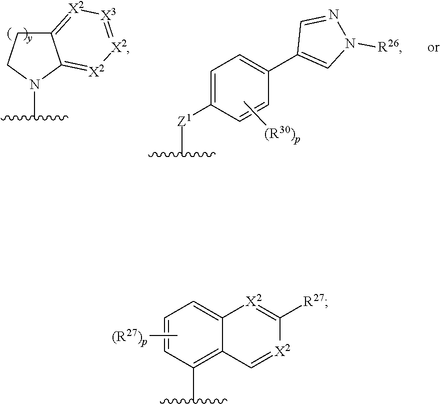
R²⁷ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, substituted or unsubstituted heteroaryl, —CN, —OH, or —OR^{a}; and
each R³⁰ is independently hydrogen, substituted or unsubstituted C₁-C₆alkyl, substituted or unsubstituted C₁-C₆fluoroalkyl, substituted or unsubstituted C₁-C₆heteroalkyl, —CN, —OH, or —OR^{a}; and
each R^{a} is independently substituted or unsubstituted C₁-C₆alkyl.

23. The compound of claim 22, or a pharmaceutically acceptable salt or solvate thereof, wherein:
each R²⁷ is independently hydrogen, —CH₃, —CH₂CH₃, —F, —CHF₂, —CF₃, —CN, —OH, —OCH₃, cyclopropyl, cyclobutyl, oxetanyl, tetrahydrofuranyl, tetrahydropyranyl, phenyl, pyrazolyl, 1-methyl pyrazolyl, pyridinyl, or pyrimidinyl.

24. The compound of claim 21, or a pharmaceutically acceptable salt or solvate thereof, wherein:
R³² is

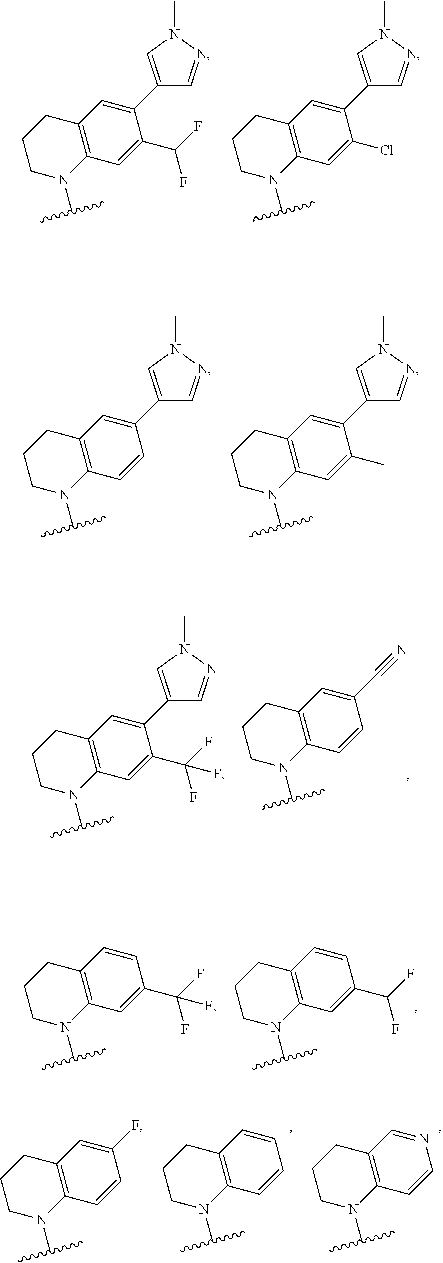

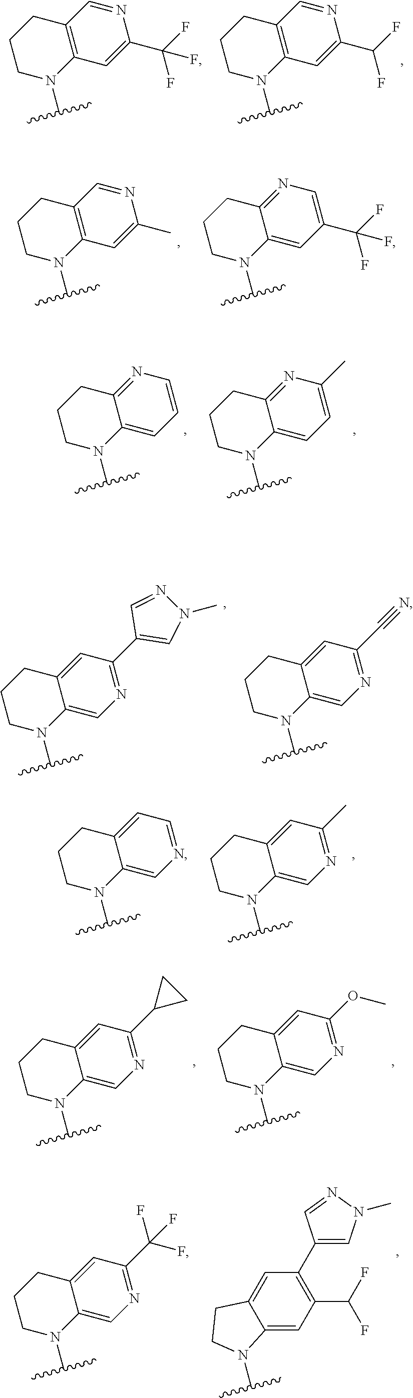

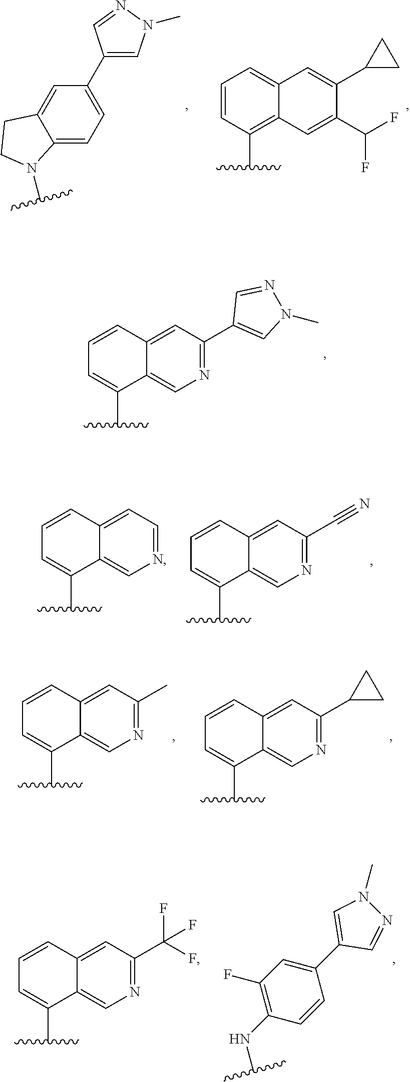

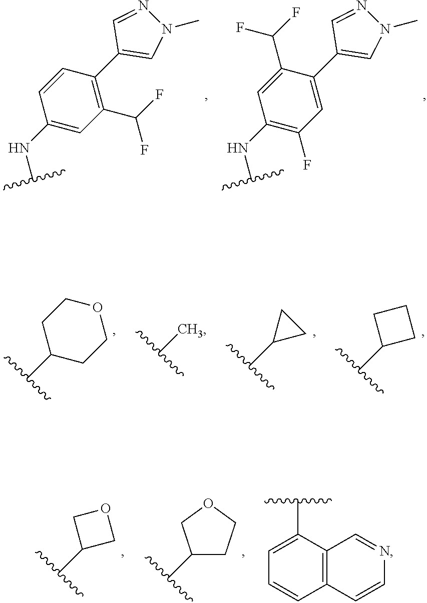

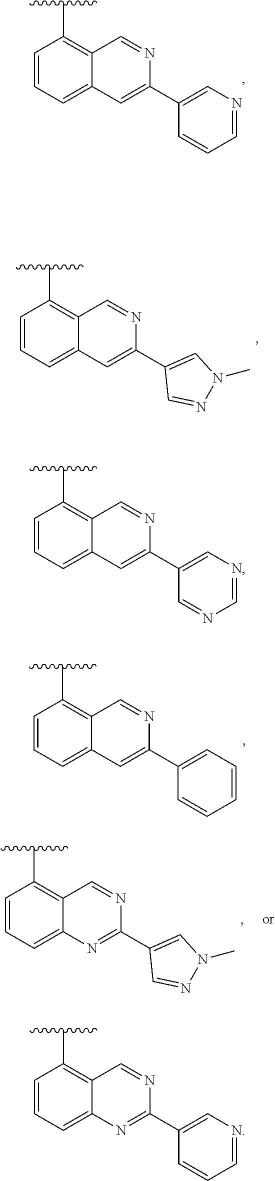 [SMILES: *c1cccc2cc(-c3cccnc3)ncc12 ; *c1cccc2cc(-c3cnn(C)c3)ncc12 ; *c1cccc2cc(-c3cncnc3)ncc12 ; *c1cccc2cc(-c3ccccc3)ncc12 ; *c1cccc2nc(-c3cnn(C)c3)ncc12 ; *c1cccc2nc(-c3cccnc3)ncc12]

25. The compound of any one of claims 12-19, or a pharmaceutically acceptable salt or solvate thereof, wherein the moiety comprising an acetyl-lysine mimetic comprises

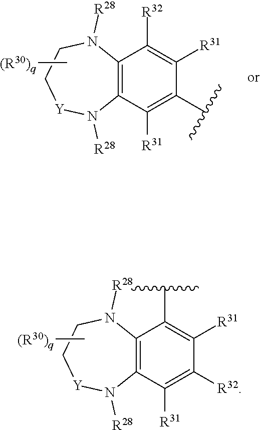

26. The compound of claim 25, or a pharmaceutically acceptable salt or solvate thereof, wherein the moiety comprising an acetyl-lysine mimetic comprises

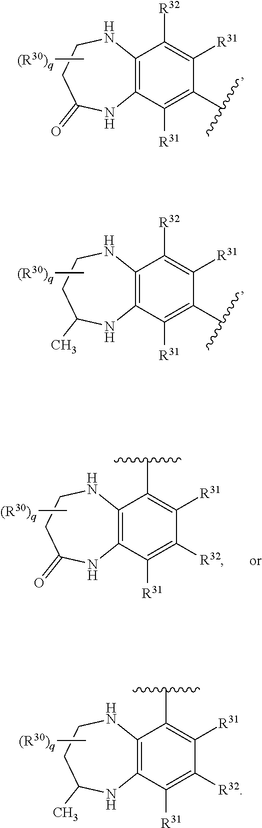

27. The compound of claim 25, or a pharmaceutically acceptable salt or solvate thereof, wherein the moiety comprising an acetyl-lysine mimetic comprises 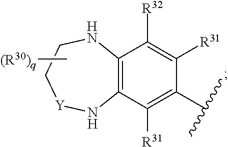
wherein:
each R²⁸ is independently hydrogen or substituted or unsubstituted C₁-C₆alkyl;
R³² is 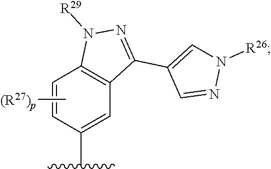
and
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂.

28. The compound of any one of claims 25-27, or a pharmaceutically acceptable salt or solvate thereof, wherein:
R³² is

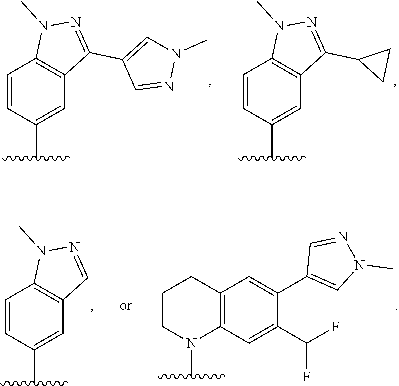 [SMILES: *c1ccc2c(c1)c(-c1cnn(C)c1)nn2C ; *c1ccc2c(c1)c(C1CC1)nn2C ; *c1ccc2c(cnn2C)c1 ; *N1CCCc2cc(-c3cnn(C)c3)c(C(F)F)cc21]

29. The compound of any one of claims 12-19, or a pharmaceutically acceptable salt or solvate thereof, wherein the moiety comprising an acetyl-lysine mimetic comprises

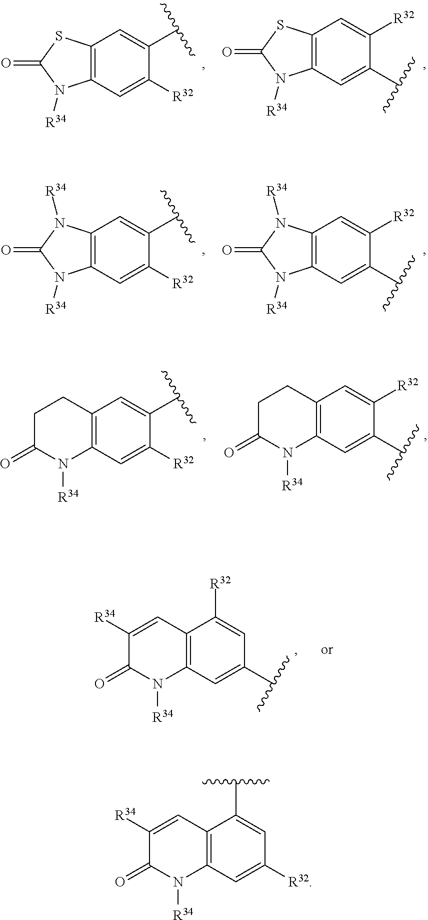

30. The compound of claim 29, or a pharmaceutically acceptable salt or solvate thereof, wherein the moiety comprising an acetyl-lysine mimetic comprises

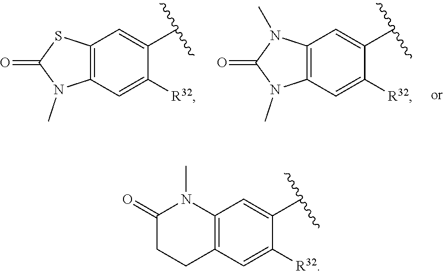

31. The compound of any one of claims 29-30, or a pharmaceutically acceptable salt or solvate thereof, wherein:
R³² is

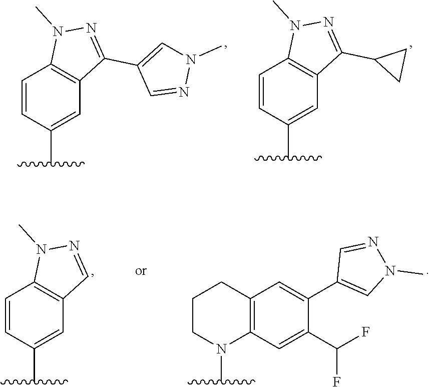 [SMILES: *c1ccc2c(c1)c(-c1cnn(C)c1)nn2C ; *c1ccc2c(c1)c(C1CC1)nn2C ; *c1ccc2c(cnn2C)c1 ; *N1CCCc2cc(-c3cnn(C)c3)c(C(F)F)cc21]

32. The compound of any one of claims 12-19, or a pharmaceutically acceptable salt or solvate thereof, wherein the moiety comprising an acetyl-lysine mimetic comprises:

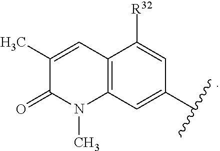

33. The compound of claim 32, or a pharmaceutically acceptable salt or solvate thereof, wherein:
R³² is

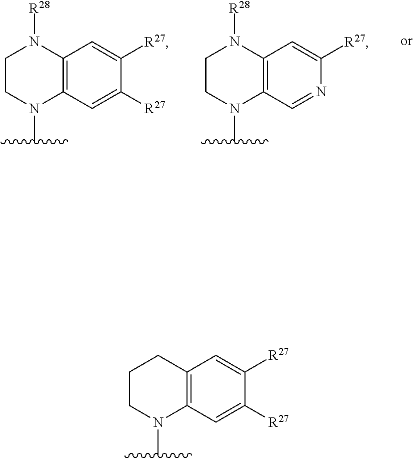

34. The compound of any one of claims 29, 30, 32, or 33, or a pharmaceutically acceptable salt or solvate thereof, wherein:
R³² is

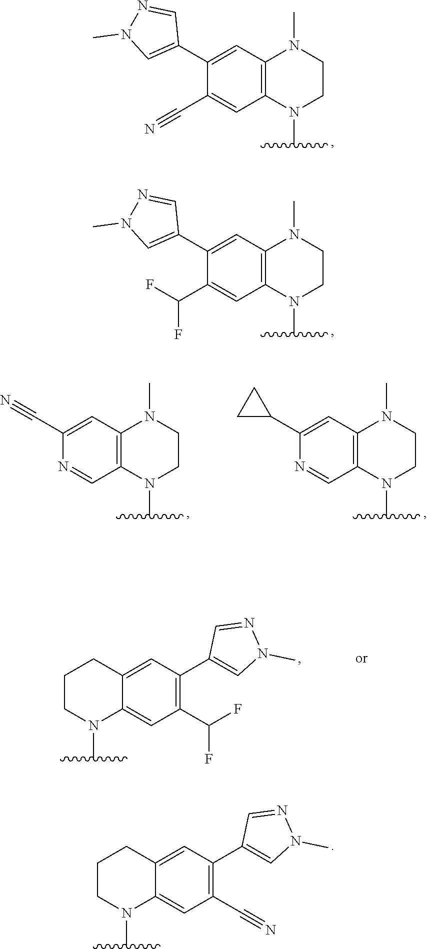 [SMILES: *N1CCN(C)c2cc(-c3cnn(C)c3)c(C#N)cc21 ; *N1CCN(C)c2cc(-c3cnn(C)c3)c(C(F)F)cc21 ; *N1CCN(C)c2cc(C#N)ncc21 ; *N1CCN(C)c2cc(C3CC3)ncc21 ; *N1CCCc2cc(-c3cnn(C)c3)c(C(F)F)cc21 ; *N1CCCc2cc(-c3cnn(C)c3)c(C#N)cc21]

35. The compound of any one of claims 12-19, or a pharmaceutically acceptable salt or solvate thereof, wherein the moiety comprising an acetyl-lysine mimetic comprises

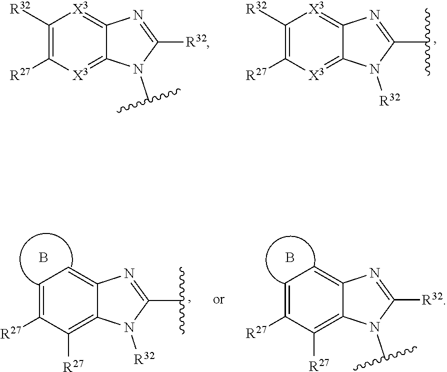

36. The compound of claim 35, or a pharmaceutically acceptable salt or solvate thereof, wherein the moiety comprising an acetyl-lysine mimetic comprise 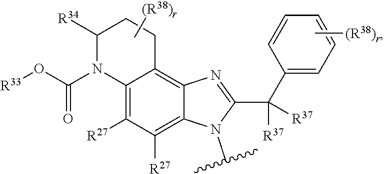
wherein:
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR^{a}, or —N(R^{b})₂;
R³³ is hydrogen, substituted or unsubstituted C₁-C₆alkyl, substituted or unsubstituted C₁-C₆fluoroalkyl, or substituted or unsubstituted C₁-C₆heteroalkyl;
R³⁴ is hydrogen or substituted or unsubstituted C₁-C₆alkyl;
each R³⁸ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, —CN, —NO₂, —OH, —OR^{a}, —OC(═O)R^{a}, —OC(═O)N(R^{b})₂, —OC(═O)OR^{a}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
r is 0, 1, 2, 3, or 4;

37. The compound of claim 35, or a pharmaceutically acceptable salt or solvate thereof, wherein:

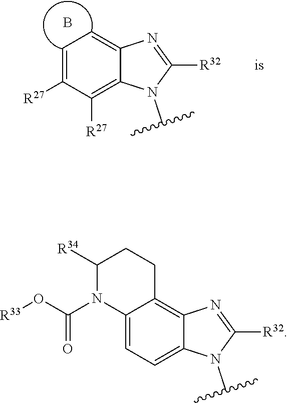

38. The compound of claim 35, or a pharmaceutically acceptable salt or solvate thereof, wherein the moiety comprising an acetyl-lysine mimetic comprises

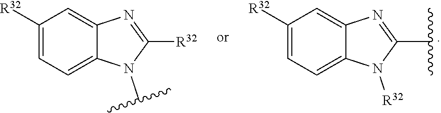

39. The compound of any one of claims 35-38, or a pharmaceutically acceptable salt or solvate thereof, wherein:
R³² is

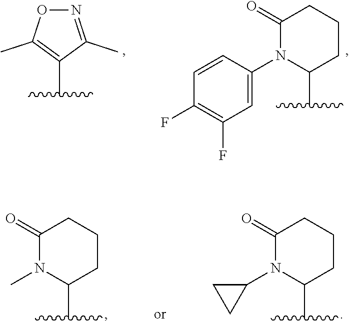

40. The compound of any one of claims 35-39, or a pharmaceutically acceptable salt or solvate thereof, wherein:
R³² is

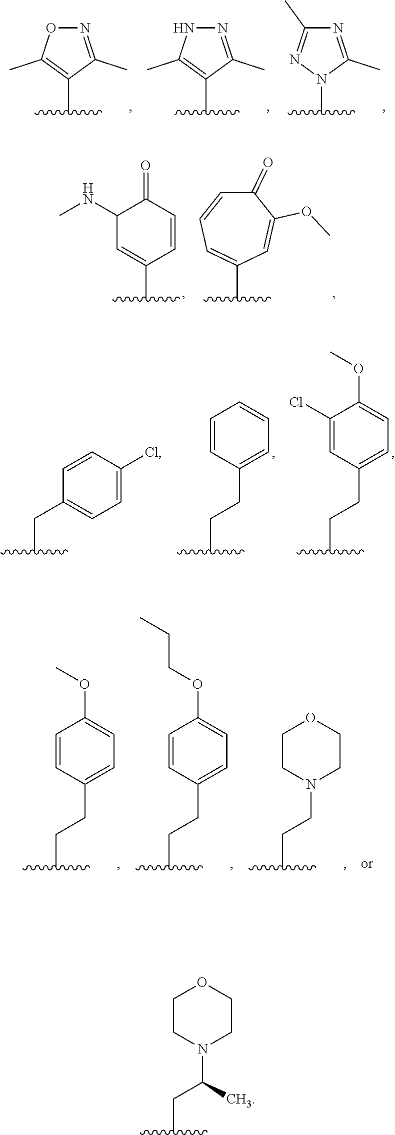

41. The compound of claim 12, or a pharmaceutically acceptable salt or solvate thereof, wherein the acetyl-lysine mimetic moiety that binds in the acetyl-lysine (KAc) binding site of the bromodomain of CBP/p300 comprises:
1-(1,4,6,7-tetrahydro-5H-pyrazolo[4,3-c]pyridin-5-yl)ethan-1-one; or N-methyl-1,4,6,7-tetrahydro-5H-pyrazolo[4,3-c]pyridine-5-carboxamide; wherein the acetyl-lysine mimetic moiety optionally further comprises:
1) a moiety at the 1-position of the 1,4,6,7-tetrahydro-5H-pyrazolo[4,3-c]pyridin-5-yl group that occupies the lipophilic shelf (LPF) region of the bromodomain of CBP/p300; or
2) a moiety that at the 3-position of the 1,4,6,7-tetrahydro-5H-pyrazolo[4,3-c]pyridin-5-yl group occupies the BC Loop region of the bromodomain of CBP/p300; or
3) both 1) and 2).

42. The compound of claim 12, or a pharmaceutically acceptable salt or solvate thereof, wherein the acetyl-lysine mimetic moiety that binds in the acetyl-lysine (KAc) binding site of the bromodomain of CBP/p300 has the structure: 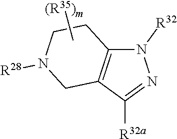
R²⁸ is —C(═O)CH₃, —C(═O)CH₂CH₃, —C(═O)NH₂, —C(═O)NH(CH₃); or —C(═O)NH(CH₂CH₃);
R³² is a moiety that occupies the BC Loop region of the bromodomain of CBP/p300;
R^{32a} is a moiety that occupies the lipophilic shelf (LPF) region of the bromodomain of CBP/p300;
each R³⁵ is independently hydrogen, C₁-C₄alkyl, or C₁-C₄fluoroalkyl;
m is 0, 1, 2, 3, or 4;
L is an optional linker;
wherein L is covalently attached to the R³² group, or at the position occupied by R³², or the R^{32a} group.

43. The compound of claim 42, or a pharmaceutically acceptable salt or solvate thereof, wherein:
R³² is hydrogen, substituted or unsubstituted C₁-C₆alkyl, substituted or unsubstituted C₃-C₁₂cycloalkyl, or substituted or unsubstituted 3- to 12-membered heterocycloalkyl;
R^{32a} is 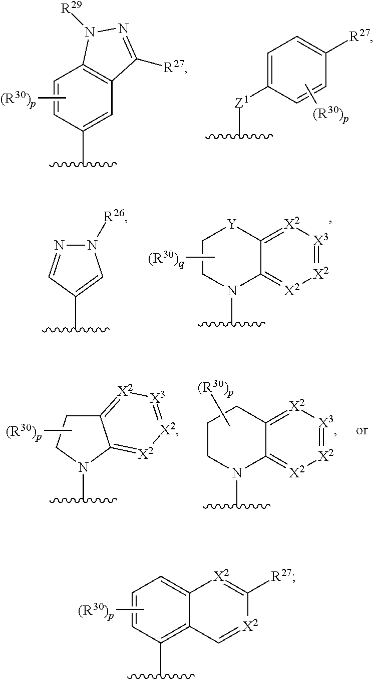
at least one X² is —CR³⁰— and at most two X² are —N—;
Z¹ is —NR^{c}— or —O—;
R^{c} is hydrogen or C₁-C₆alkyl;
R²⁶ is hydrogen, substituted or unsubstituted C₁-C₄alkyl, C₁-C₄fluoroalkyl, substituted or unsubstituted C₃-C₈cycloalkyl, or substituted or unsubstituted 3- to 8-membered heterocycloalkyl;
X³ is —CR²⁷— or —N—;
R²⁷ is hydrogen, halogen, substituted or unsubstituted C₁-C₄alkyl, C₁-C₄fluoroalkyl, substituted or unsubstituted C₃-C₈cycloalkyl, substituted or unsubstituted 3- to 8-membered heterocycloalkyl, substituted or unsubstituted phenyl, substituted or unsubstituted monocyclic heteroaryl, —CN, —OH, —OR^{a}, —N(R^{b})₂, —NR^{b}C(═O)R^{a}, —C(═O)R^{b}, —C(═O)OR^{b}, or —C(═O)N(R^{b})₂;
or R²⁷ is 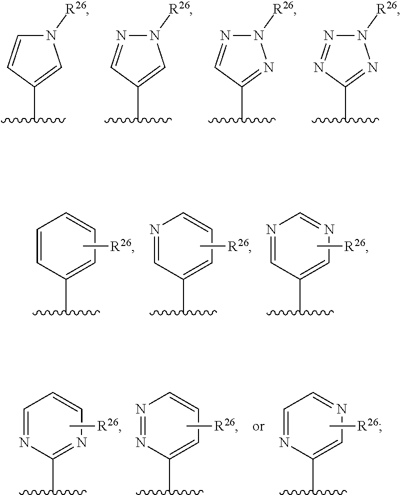
p is 0, 1, 2, or 3;
R²⁹ is hydrogen, substituted or unsubstituted C₁-C₄alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
Y is —C(R³⁰)₂— or N(R²⁸)—;
each R³⁰ is independently hydrogen, halogen, C₁-C₄alkyl, C₁-C₄fluoroalkyl, substituted or unsubstituted C₁-C₆heteroalkyl, —CN, —OH, or —OR^{a};
q is 0, 1, 2, 3, or 4;
each R^{a} is independently C₁-C₄alkyl, C₁-C₆fluoroalkyl, substituted or unsubstituted C₃-C₆cycloalkyl, substituted or unsubstituted monocyclic 3- to 6-membered heterocycloalkyl, substituted or unsubstituted phenyl, or substituted or unsubstituted monocyclic heteroaryl;
each R^{b} is independently hydrogen, C₁-C₄alkyl, C₁-C₄fluoroalkyl, substituted or unsubstituted C₃-C₆cycloalkyl, substituted or unsubstituted monocyclic 3- to 6-membered heterocycloalkyl, substituted or unsubstituted phenyl, or substituted or unsubstituted monocyclic heteroaryl;
or two R^{b} on the same N atom are taken together with the N atom to which they are attached to form a substituted or unsubstituted N-containing heterocycle.

44. The compound of any one of claims 42-43, or a pharmaceutically acceptable salt or solvate thereof, wherein:
R²⁷ is hydrogen, halogen, C₁-C₄alkyl, C₁-C₄fluoroalkyl, substituted or unsubstituted C₃-C₈cycloalkyl, substituted or unsubstituted 3- to 8-membered heterocycloalkyl, substituted or unsubstituted phenyl, substituted or unsubstituted monocyclic 5- or 6-membered heteroaryl, —CN, —OH, —OR^{a}, —N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
or R²⁷ is

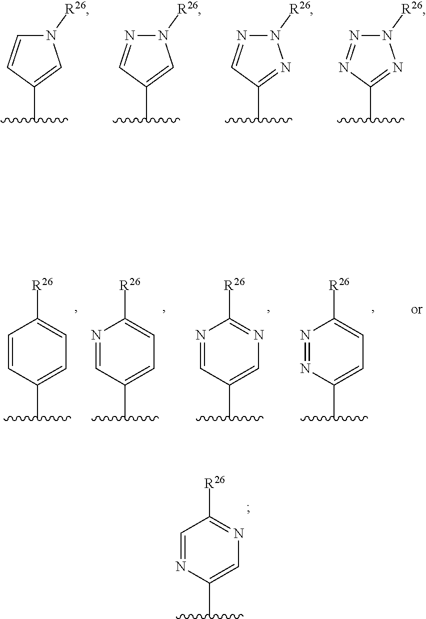

45. The compound of any one of claims 42-44, or a pharmaceutically acceptable salt or solvate thereof, wherein:
R³² is 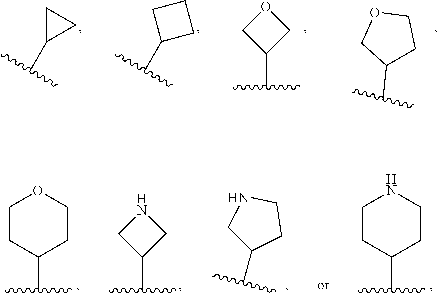
each of which is unsubstituted or substituted with F, Cl, Br, —CH₃, —CD₃, —CH₂CH₃, —CH₂F, —CHF₂, —CF₃, —CH₂CH₂F, —CH₂CHF₂, —CH₂CF₃, —CN, —C(═O)CH₃, —C(═O)CH₂CH₃, —C(═O)CH₂F, —C(═O)CHF₂, —C(═O)CF₃, —C(═O)CH₂CH₂F, —C(═O)CH₂CHF₂, —C(═O)CH₂CF₃, —C(═O)CD₃, or —SO₂CH₃, —SO₂CH₂CH₃, —SO₂CD₃, or —SO₂CH₂CD₃.
R^{32a} is 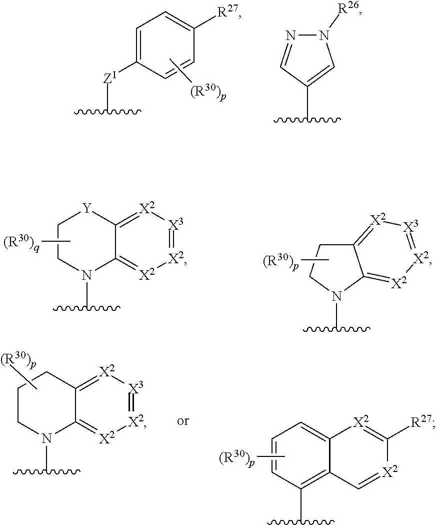
at least one X² is —CR³⁰— and at most two X² are —N—;
each R²⁷ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, substituted or unsubstituted heteroaryl, —CN, —OH, or —OR^{a}; and
each R^{a} is independently substituted or unsubstituted C₁-C₆alkyl.

46. The compound of any one of claims 42-45, or a pharmaceutically acceptable salt or solvate thereof, wherein:
R²⁷ is hydrogen, —CH₃, —CH₂CH₃, —CH₂CH₂CH₃, —CH₂(CH₃)₂, —(CH₃)₃, —F, —CHF₂, —CF₃, —CN, —OH, —OCH₃, cyclopropyl, cyclobutyl, oxetanyl, tetrahydrofuranyl, tetrahydropyranyl, unsubstituted or substituted phenyl, unsubstituted or substituted pyrazolyl, unsubstituted or substituted pyridinyl, or unsubstituted or substituted pyrimidinyl; or
R²⁷ is 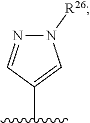
R³² is 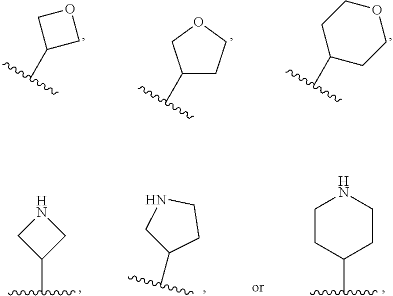 [SMILES: *C1COC1 ; *C1CCOC1 ; *C1CCOCC1 ; *C1CNC1 ; *C1CCNC1 ; *C1CCNCC1]
each of which is unsubstituted or substituted with F, Cl, Br, —CH₃, —CD₃, —CH₂CH₃, —CH₂F, —CHF₂, —CF₃, CN, —C(═O)CH₃, —C(═O)CH₂F, —C(═O)CHF₂, —C(═O)CF₃, —C(═O)CD₃.

47. The compound of claim 42-45, or a pharmaceutically acceptable salt or solvate thereof, wherein:
R³² is 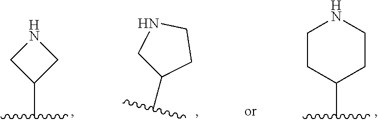 [SMILES: *C1CNC1 ; *C1CCNC1 ; *C1CCNCC1]
wherein the optional linker is covalently attached to the nitrogen of R³² group;
or R³² is absent and L is covalently attached to the acetyl-lysine mimetic moiety at the position occupied by R³²; and
R^{32a} is

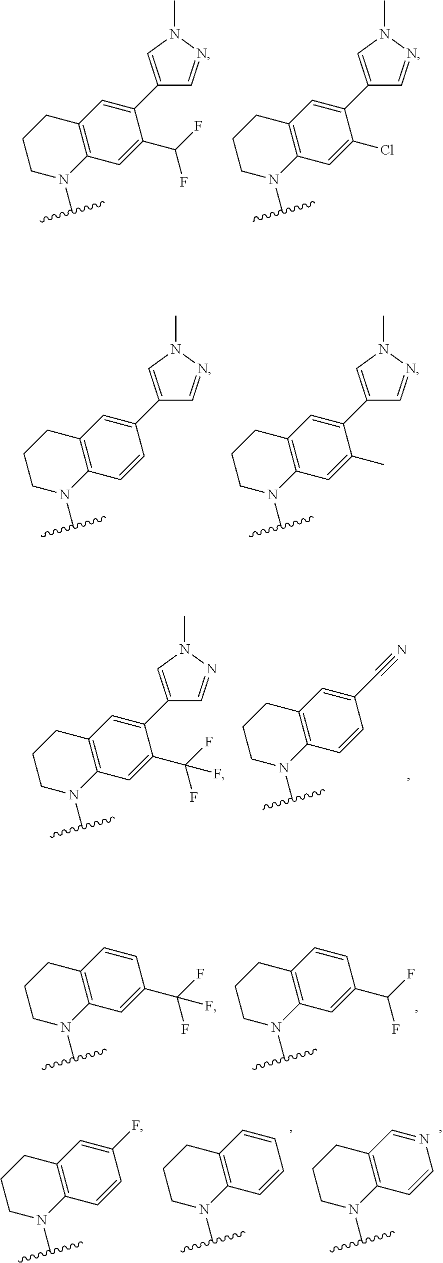

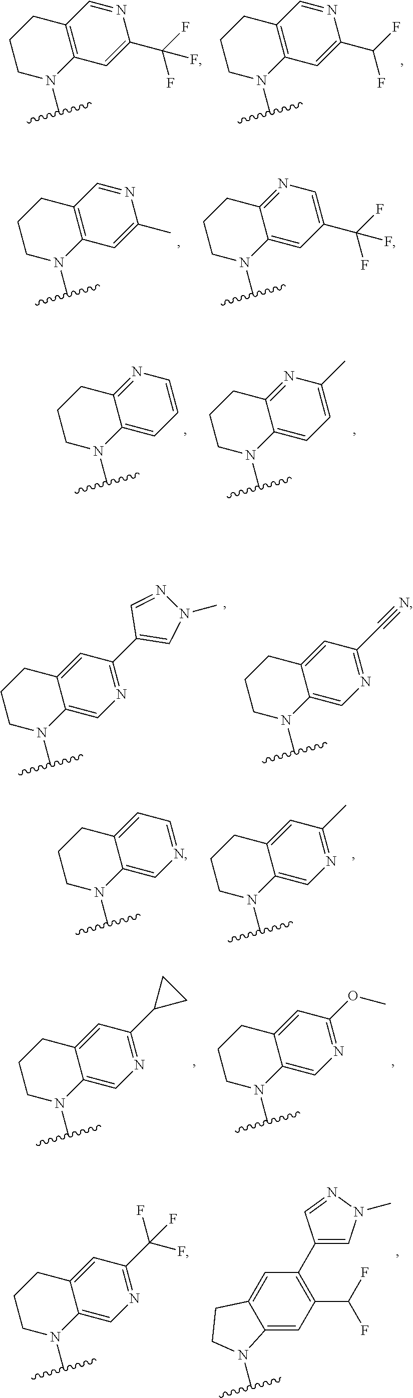

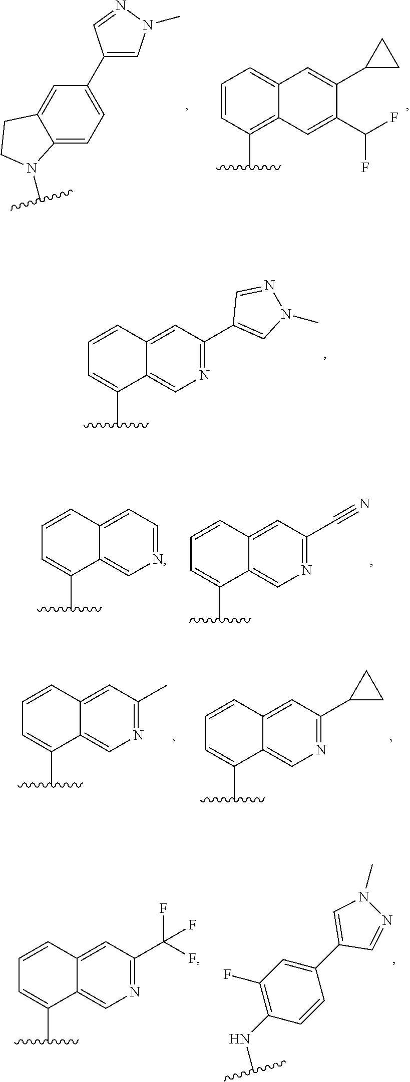

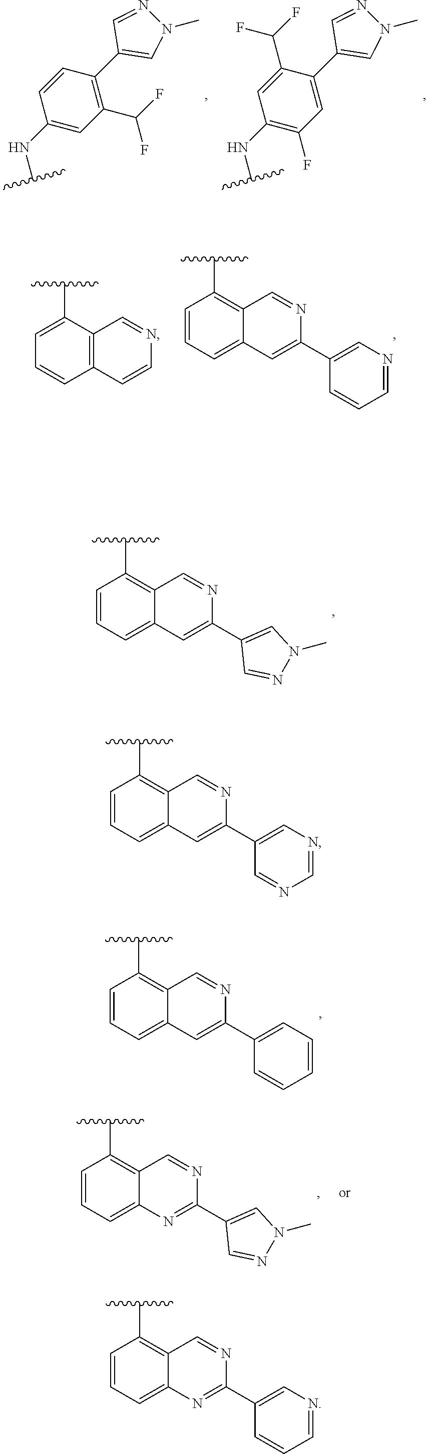

48. The compound of any one of claims 42-45, or a pharmaceutically acceptable salt or solvate thereof, wherein:
R³² is 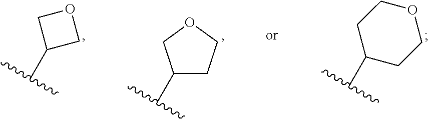 [SMILES: *C1COC1 ; *C1CCOC1 ; *C1CCOCC1]
R³² is 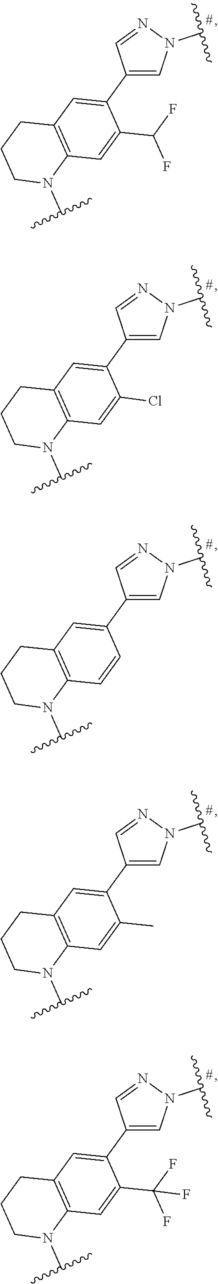 [SMILES: *N1CCCc2cc(-c3cnn(*)c3)c(C(F)F)cc21 ; *N1CCCc2cc(-c3cnn(*)c3)c(Cl)cc21 ; *N1CCCc2cc(-c3cnn(*)c3)ccc21 ; *N1CCCc2cc(-c3cnn(*)c3)c(C)cc21 ; *N1CCCc2cc(-c3cnn(*)c3)c(C(F)(F)F)cc21] 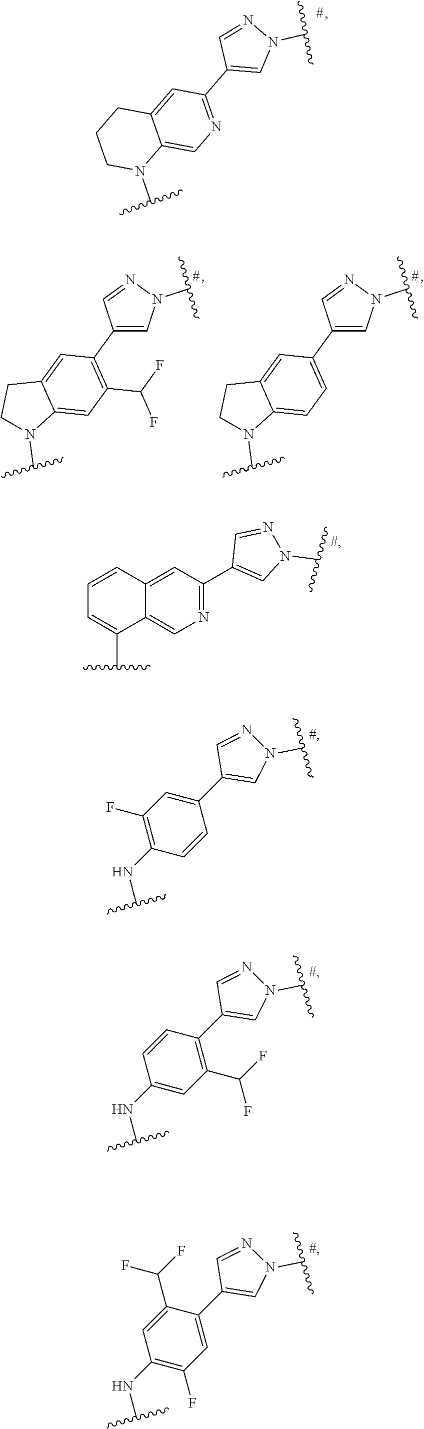 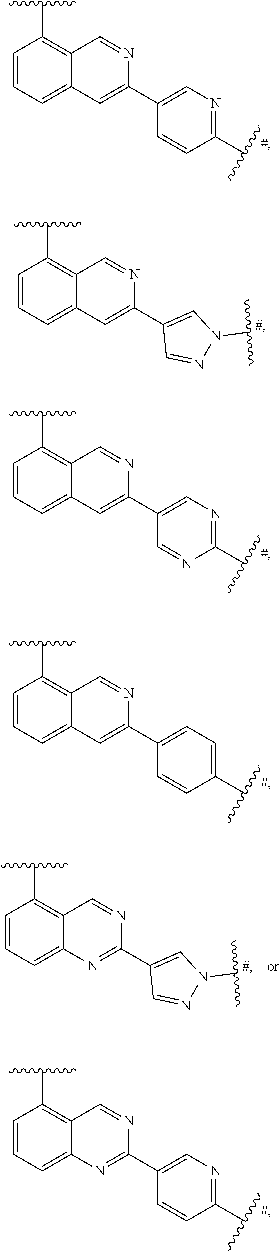 [SMILES: *c1ccc(-c2cc3cccc(*)c3cn2)cn1 ; *c1cccc2cc(-c3cnn(*)c3)ncc12 ; *c1ncc(-c2cc3cccc(*)c3cn2)cn1 ; *c1ccc(-c2cc3cccc(*)c3cn2)cc1 ; *c1cccc2nc(-c3cnn(*)c3)ncc12 ; *c1ccc(-c2ncc3c(*)cccc3n2)cn1]
wherein [symbol]
is the point of attachment to the optional linker.

49. The compound of claim 42, or a pharmaceutically acceptable salt or solvate thereof, wherein:
R²⁸ is —C(═O)CH₃ or —C(═O)NH(CH₃);
each R³⁵ is independently hydrogen, —CH₃, —CH₂F, —CHF₂, —CF₃;
m is 0, 1, or 2;
R³² is 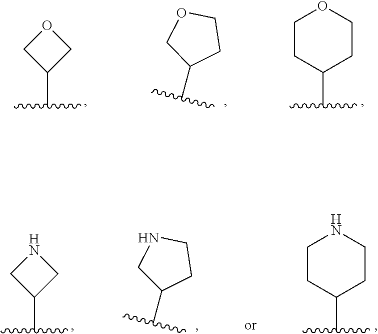 [SMILES: *C1COC1 ; *C1CCOC1 ; *C1CCOCC1 ; *C1CNC1 ; *C1CCNC1 ; *C1CCNCC1]
each of which is unsubstituted or substituted with F, —CH₃, —CH₂F, —CHF₂, or —CF₃;
R^{32a} is 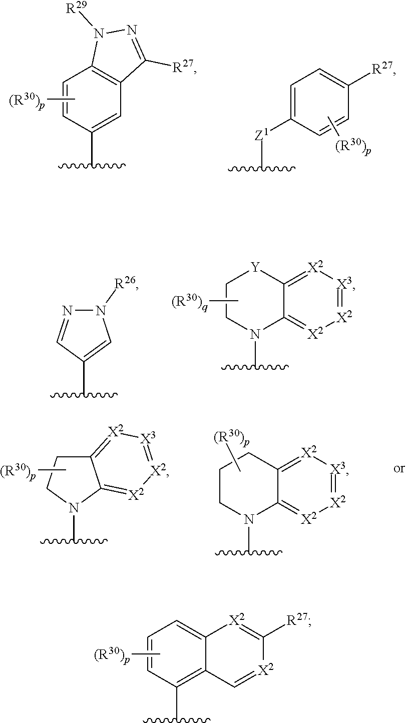
at least one X² is —CR³⁰— and at most two X² are —N—;
R²⁶ is hydrogen, C₁-C₄alkyl, C₁-C₄fluoroalkyl, substituted or unsubstituted C₃-C₆cycloalkyl, or substituted or unsubstituted 3- to 6-membered heterocycloalkyl;
R²⁷ is hydrogen, halogen, C₁-C₄alkyl, C₁-C₄fluoroalkyl, substituted or unsubstituted C₃-C₆cycloalkyl, substituted or unsubstituted 3- to 6-membered heterocycloalkyl, substituted or unsubstituted phenyl, substituted or unsubstituted monocyclic 5- or 6-membered heteroaryl, —CN, —OH, —OR^{a}, or —N(R^{b})₂;
or R²⁷ is 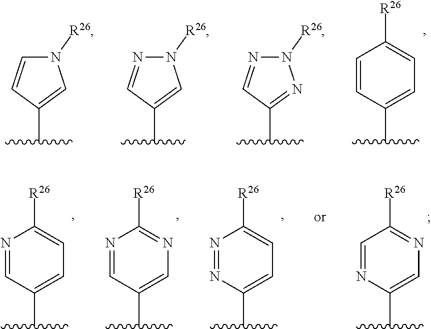
each R³⁰ is independently hydrogen, halogen, C₁-C₄alkyl, C₁-C₄fluoroalkyl, —CN, —OH, or —OR^{a};
R^{a} is C₁-C₄alkyl;
each R^{b} is independently hydrogen or C₁-C₆alkyl;
wherein L is covalently attached to the R³² group, or at the position occupied by R²⁶, or at the position occupied by R²⁷.

50. The compound of claim 49, or a pharmaceutically acceptable salt or solvate thereof, wherein:
R³² is 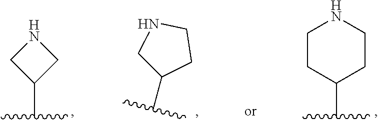 [SMILES: *C1CNC1 ; *C1CCNC1 ; *C1CCNCC1]
wherein the optional linker is covalently attached to the nitrogen of R³² group;
or R³² is absent and the optional linker is covalently attached to the acetyl-lysine mimetic moiety at the position occupied by R³²;
R^{32a} is 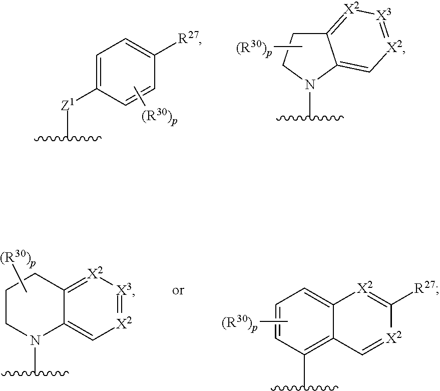
at least one X² is —CR³⁰— and at most two X² are —N—;
X³ is —CR²⁷— or —N—;
Z¹ is —NH— or —O—;
R²⁶ is hydrogen, C₁-C₄alkyl, C₁-C₄fluoroalkyl, substituted or unsubstituted C₃-C₆cycloalkyl, or substituted or unsubstituted 3- to 6-membered heterocycloalkyl;
R²⁷ is hydrogen, halogen, C₁-C₄alkyl, C₁-C₄fluoroalkyl, substituted or unsubstituted C₃-C₆cycloalkyl, substituted or unsubstituted 3- to 6-membered heterocycloalkyl, substituted or unsubstituted phenyl, substituted or unsubstituted monocyclic 5- or 6-membered heteroaryl, —CN, —OH, —OR^{a}, or —N(R^{b})₂;
or R²⁷ is 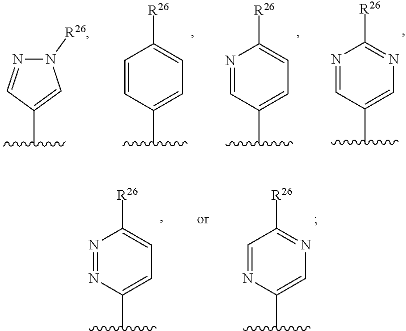
each R³⁰ is independently hydrogen, halogen, C₁-C₄alkyl, C₁-C₄fluoroalkyl, —CN, —OH, or —OR^{a}.

51. The compound of claim 49, or a pharmaceutically acceptable salt or solvate thereof, wherein:
R³² is 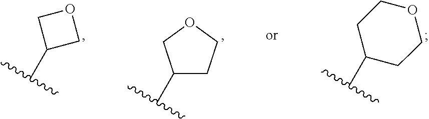 [SMILES: *C1COC1 ; *C1CCOC1 ; *C1CCOCC1]
R^{32a} is 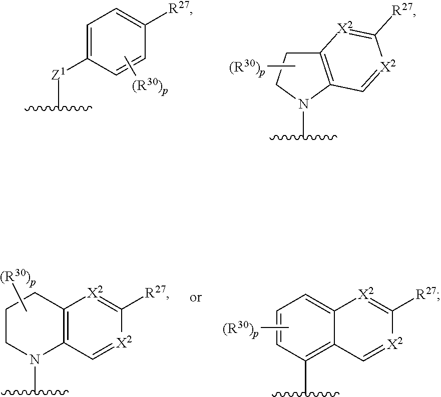
R²⁷ is 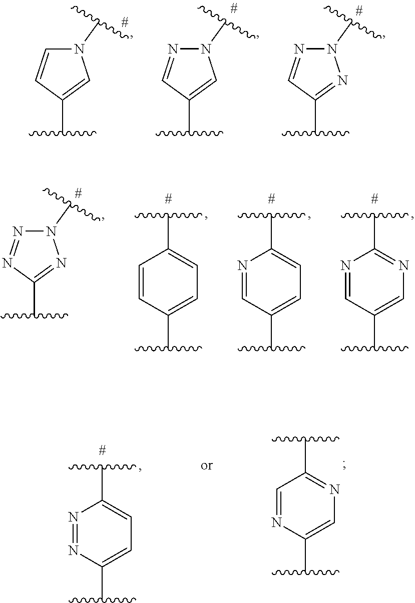
Z¹ is —NH— or —O—; [symbol]
is the point of attachment to the optional linker.

52. The compound of any one of claims 9-14, or a pharmaceutically acceptable salt or solvate thereof, wherein SB-CBP/p300 has the structure of Formula (IIIb): 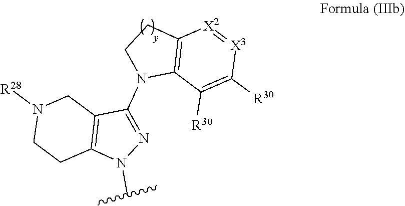
wherein:
each X² is independently —CR³⁰— or —N—;
X³ is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
each R³⁰ is independently hydrogen, halogen, C₁-C₄alkyl, C₁-C₄fluoroalkyl, —CN, —OH, or —OR^{a};
y is 1 or 2;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

53. The compound of any one of claims 9-14, or a pharmaceutically acceptable salt or solvate thereof, wherein SB-CBP/p300 has the structure of Formula (IIIc): 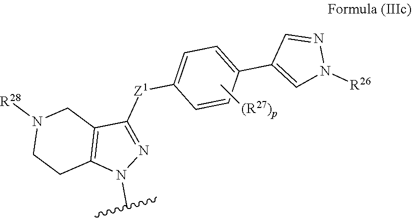
wherein:
R²⁶ is hydrogen, substituted or unsubstituted C₁-C₆alkyl, substituted or unsubstituted C₃-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{a}, or —C(═O)N(R^{b})₂;
Z¹ is —NR^{c}—, —O—, or —S—;
R^{c} is hydrogen or substituted or unsubstituted C₁-C₆alkyl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

54. The compound of any one of claims 9-14, or a pharmaceutically acceptable salt or solvate thereof, wherein SB-CBP/p300 has the structure of Formula (IIId-1) or (IIId-2): 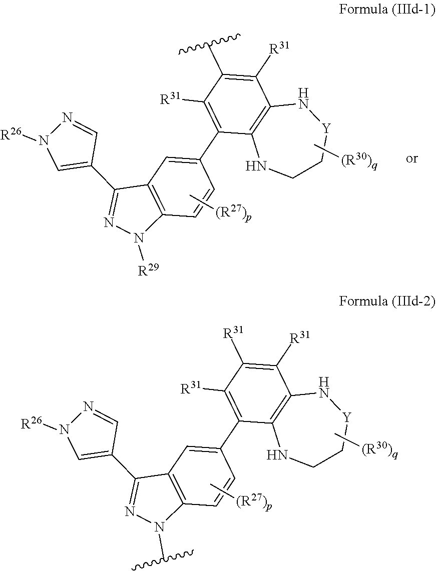
wherein:
R²⁶ is hydrogen, substituted or unsubstituted C₁-C₆alkyl, substituted or unsubstituted C₁-C₆heteroalkyl, substituted or unsubstituted C₃-C₈cycloalkyl, or substituted or unsubstituted 3- to 8-membered heterocycloalkyl;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁹ is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
each R³⁰ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
q is 0, 1, 2, 3, or 4;
each R³¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₁-C₆fluoroalkyl, —CN, —NO₂, —OH, —OR^{a}, OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)NR^{b}, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
Y is —C(R³⁰)₂— or C(═O);
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

55. The compound of any one of claims 9-14, or a pharmaceutically acceptable salt or solvate thereof, wherein SB-CBP/p300 has the structure of Formula (IIIe): 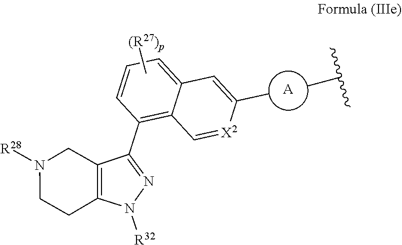
wherein:
X² is —CR²⁷— or —N—;
each R²⁷ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, OC(═O)R^{a}, —OC(═O)N(R^{b})₂, —OC(═O)OR^{a}, —OC(═O)N(R^{b})₂, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b})₂;
p is 0, 1, 2, or 3;
R²⁸ is hydrogen, substituted or unsubstituted C₁-C₆alkyl, substituted or unsubstituted C₁-C₆fluoroalkyl, —C(═O)R^{b}, or —C(═O)N(R^{b})₂;
R³² is hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted 3- to 8-membered heterocycloalkyl;
Ring A is absent or substituted or unsubstituted C₃-C₈cycloalkyl, substituted or unsubstituted 3- to 8-membered heterocycloalkyl, substituted or unsubstituted aryl, or substituted or unsubstituted heteroaryl;
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

56. The compound of any one of claims 9-14, or a pharmaceutically acceptable salt or solvate thereof, wherein SB-CBP/p300 has one of the following structures:

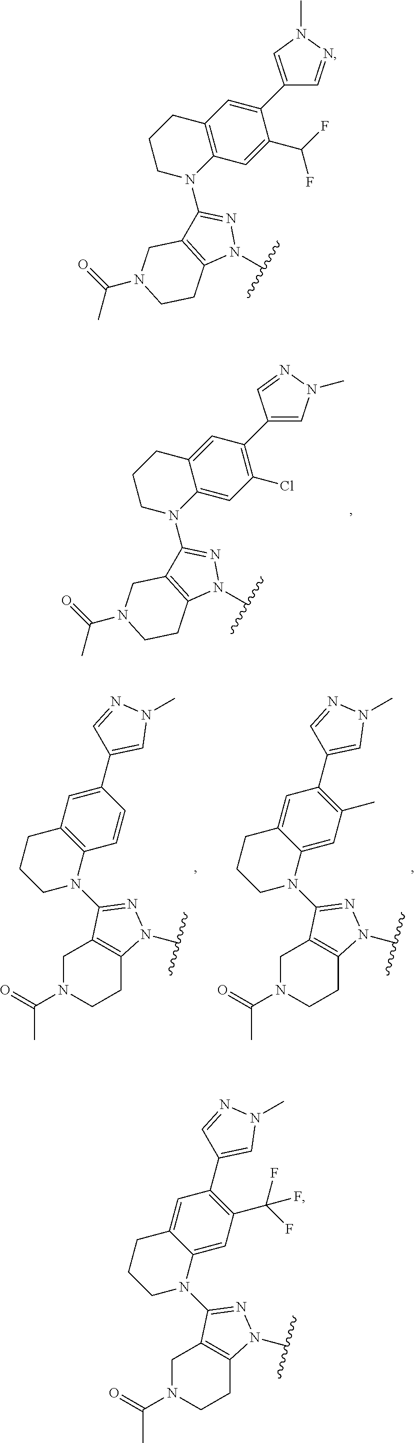 [SMILES: *n1nc(N2CCCc3cc(-c4cnn(C)c4)c(C(F)F)cc32)c2c1CCN(C(C)=O)C2 ; *n1nc(N2CCCc3cc(-c4cnn(C)c4)c(Cl)cc32)c2c1CCN(C(C)=O)C2 ; *n1nc(N2CCCc3cc(-c4cnn(C)c4)ccc32)c2c1CCN(C(C)=O)C2 ; *n1nc(N2CCCc3cc(-c4cnn(C)c4)c(C)cc32)c2c1CCN(C(C)=O)C2 ; *n1nc(N2CCCc3cc(-c4cnn(C)c4)c(C(F)(F)F)cc32)c2c1CCN(C(C)=O)C2]

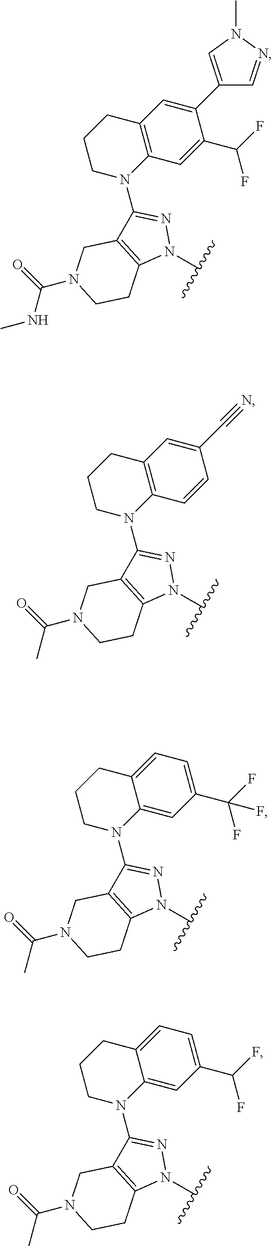 [SMILES: *n1nc(N2CCCc3cc(-c4cnn(C)c4)c(C(F)F)cc32)c2c1CCN(C(=O)NC)C2 ; *n1nc(N2CCCc3cc(C#N)ccc32)c2c1CCN(C(C)=O)C2 ; *n1nc(N2CCCc3ccc(C(F)(F)F)cc32)c2c1CCN(C(C)=O)C2 ; *n1nc(N2CCCc3ccc(C(F)F)cc32)c2c1CCN(C(C)=O)C2]

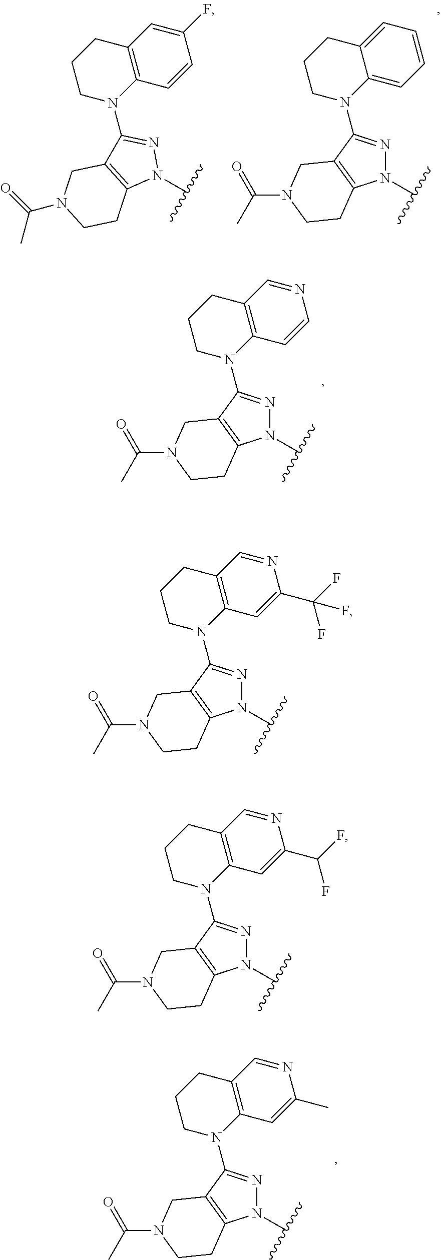 [SMILES: *n1nc(N2CCCc3cc(F)ccc32)c2c1CCN(C(C)=O)C2 ; *n1nc(N2CCCc3ccccc32)c2c1CCN(C(C)=O)C2 ; *n1nc(N2CCCc3cnccc32)c2c1CCN(C(C)=O)C2 ; *n1nc(N2CCCc3cnc(C(F)(F)F)cc32)c2c1CCN(C(C)=O)C2 ; *n1nc(N2CCCc3cnc(C(F)F)cc32)c2c1CCN(C(C)=O)C2 ; *n1nc(N2CCCc3cnc(C)cc32)c2c1CCN(C(C)=O)C2]

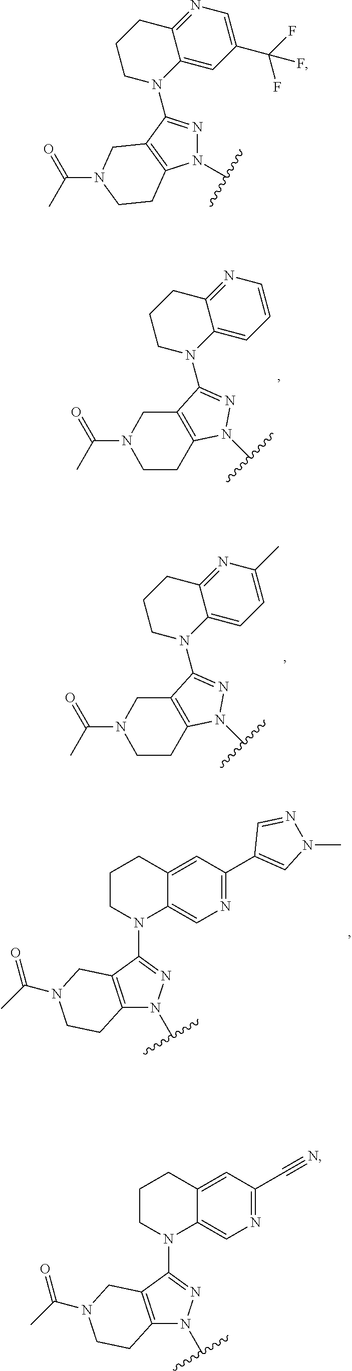

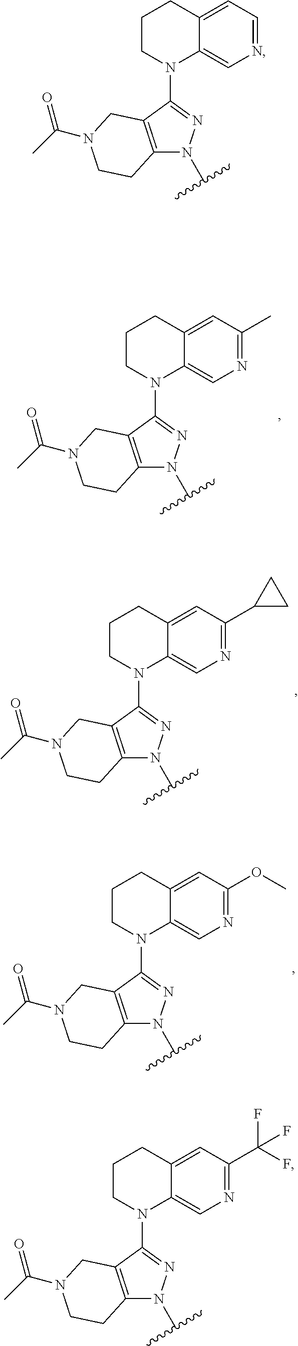

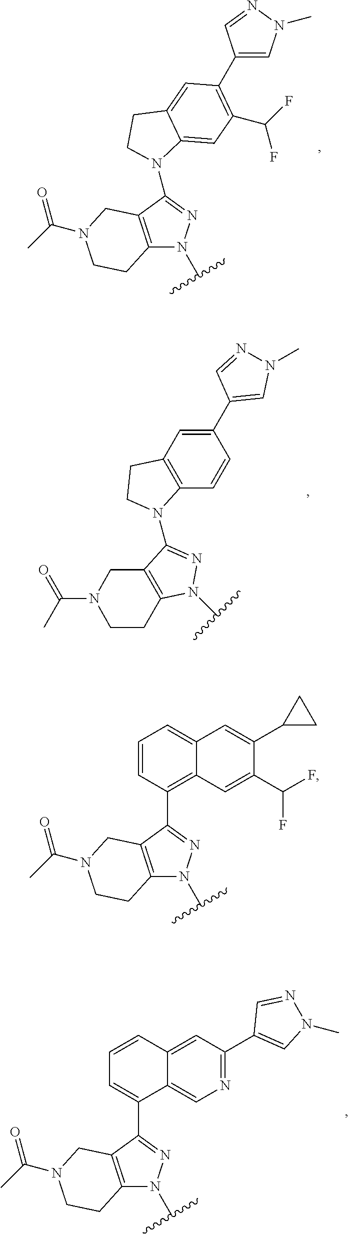

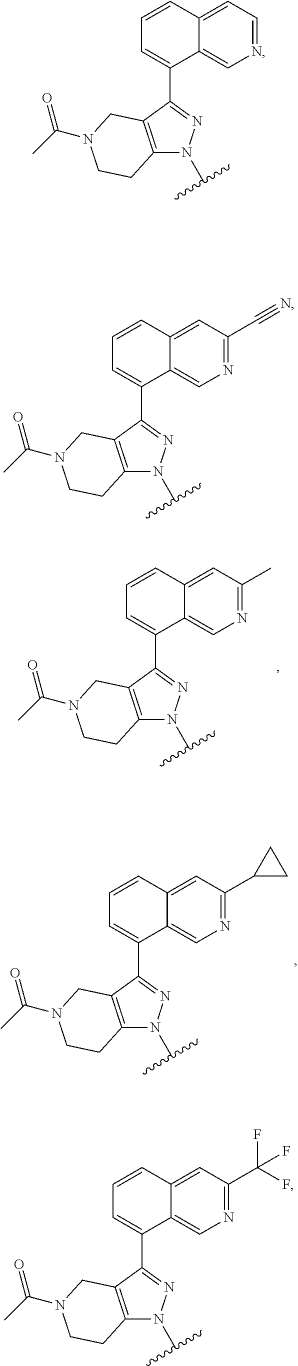

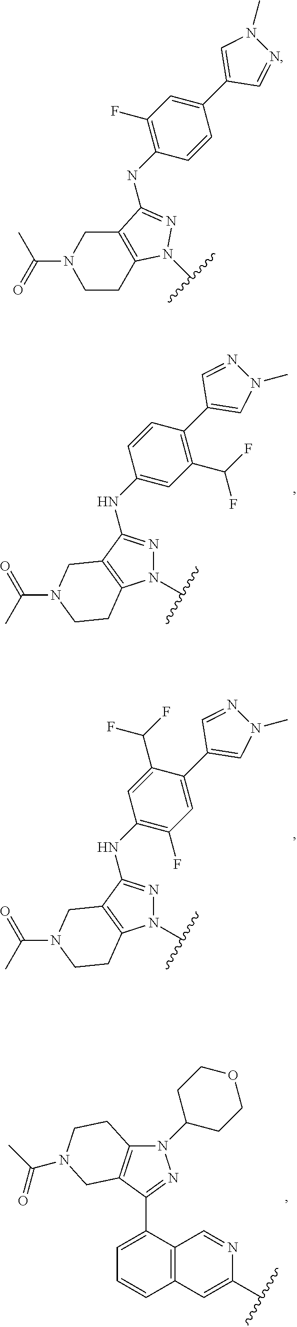

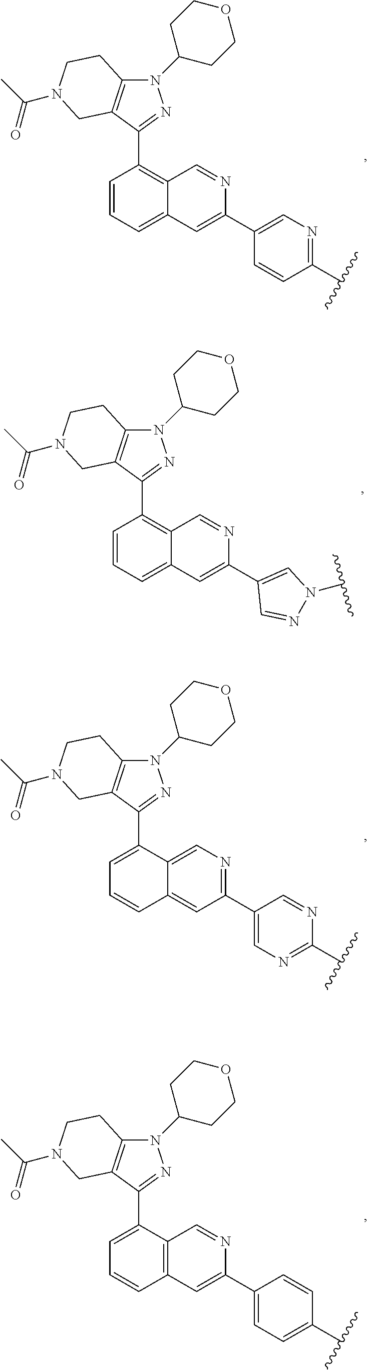 [SMILES: *c1ccc(-c2cc3cccc(-c4nn(C5CCOCC5)c5c4CN(C(C)=O)CC5)c3cn2)cn1 ; *n1cc(-c2cc3cccc(-c4nn(C5CCOCC5)c5c4CN(C(C)=O)CC5)c3cn2)cn1 ; *c1ncc(-c2cc3cccc(-c4nn(C5CCOCC5)c5c4CN(C(C)=O)CC5)c3cn2)cn1 ; *c1ccc(-c2cc3cccc(-c4nn(C5CCOCC5)c5c4CN(C(C)=O)CC5)c3cn2)cc1]

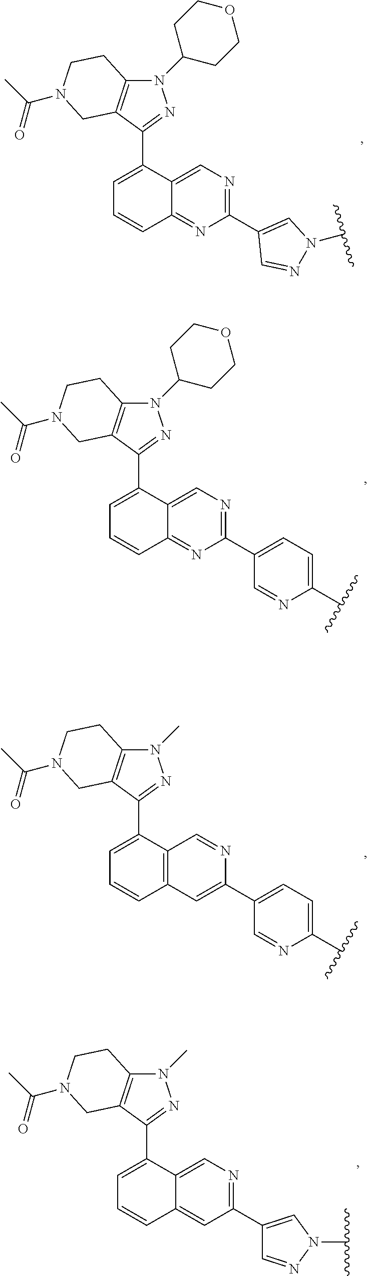 [SMILES: *n1cc(-c2ncc3c(-c4nn(C5CCOCC5)c5c4CN(C(C)=O)CC5)cccc3n2)cn1 ; *c1ccc(-c2ncc3c(-c4nn(C5CCOCC5)c5c4CN(C(C)=O)CC5)cccc3n2)cn1 ; *c1ccc(-c2cc3cccc(-c4nn(C)c5c4CN(C(C)=O)CC5)c3cn2)cn1 ; *n1cc(-c2cc3cccc(-c4nn(C)c5c4CN(C(C)=O)CC5)c3cn2)cn1]

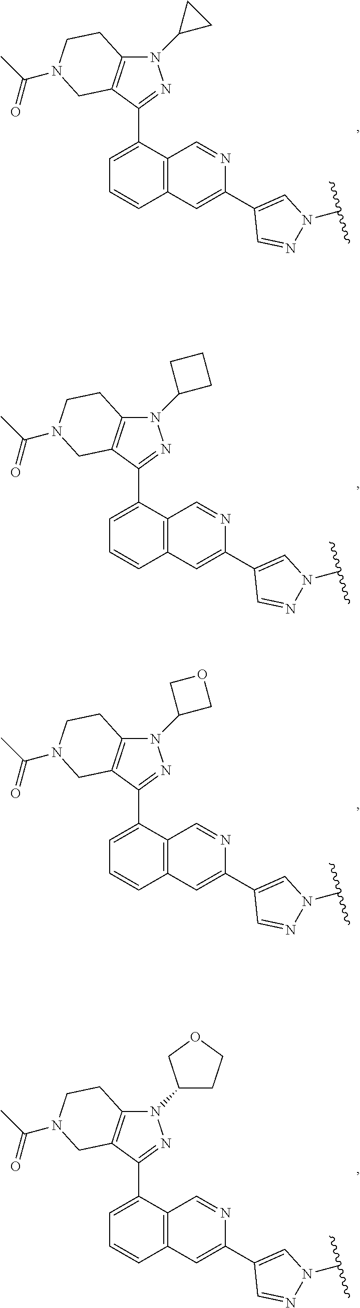 [SMILES: *n1cc(-c2cc3cccc(-c4nn(C5CC5)c5c4CN(C(C)=O)CC5)c3cn2)cn1 ; *n1cc(-c2cc3cccc(-c4nn(C5CCC5)c5c4CN(C(C)=O)CC5)c3cn2)cn1 ; *n1cc(-c2cc3cccc(-c4nn(C5COC5)c5c4CN(C(C)=O)CC5)c3cn2)cn1 ; *n1cc(-c2cc3cccc(-c4nn([C@H]5CCOC5)c5c4CN(C(C)=O)CC5)c3cn2)cn1]

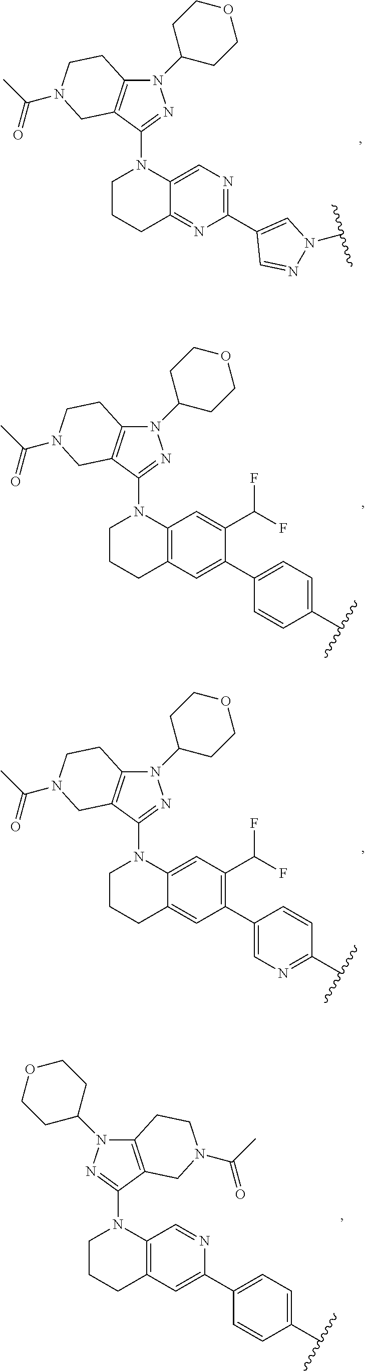 [SMILES: *n1cc(-c2ncc3c(n2)CCCN3c2nn(C3CCOCC3)c3c2CN(C(C)=O)CC3)cn1 ; *c1ccc(-c2cc3c(cc2C(F)F)N(c2nn(C4CCOCC4)c4c2CN(C(C)=O)CC4)CCC3)cc1 ; *c1ccc(-c2cc3c(cc2C(F)F)N(c2nn(C4CCOCC4)c4c2CN(C(C)=O)CC4)CCC3)cn1 ; *c1ccc(-c2cc3c(cn2)N(c2nn(C4CCOCC4)c4c2CN(C(C)=O)CC4)CCC3)cc1]

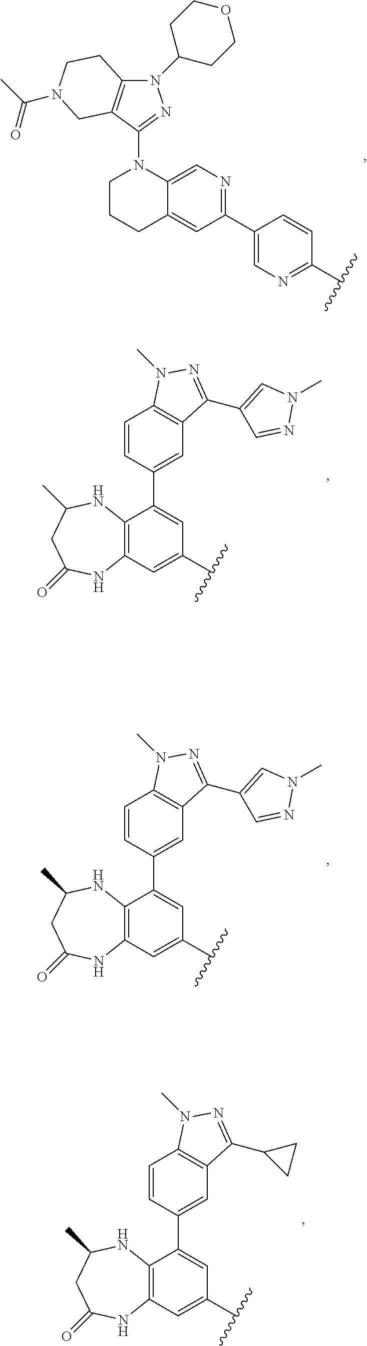 [SMILES: *c1ccc(-c2cc3c(cn2)N(c2nn(C4CCOCC4)c4c2CN(C(C)=O)CC4)CCC3)cn1 ; *c1cc2c(c(-c3ccc4c(c3)c(-c3cnn(C)c3)nn4C)c1)NC(C)CC(=O)N2 ; *c1cc2c(c(-c3ccc4c(c3)c(-c3cnn(C)c3)nn4C)c1)N[C@H](C)CC(=O)N2 ; *c1cc2c(c(-c3ccc4c(c3)c(C3CC3)nn4C)c1)N[C@H](C)CC(=O)N2]

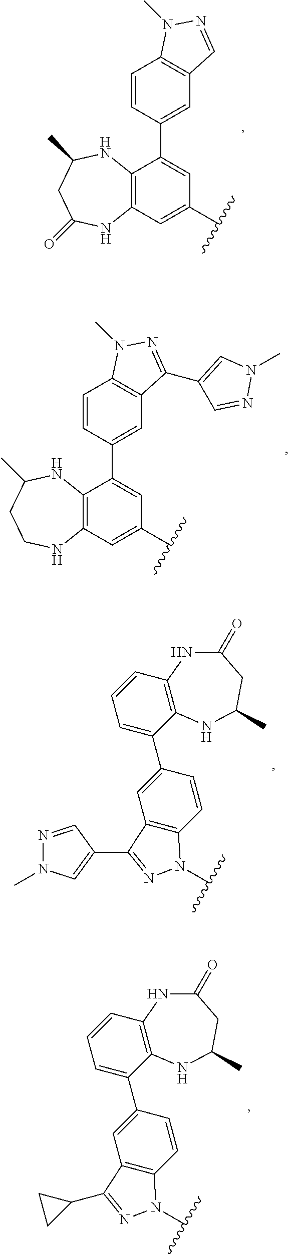

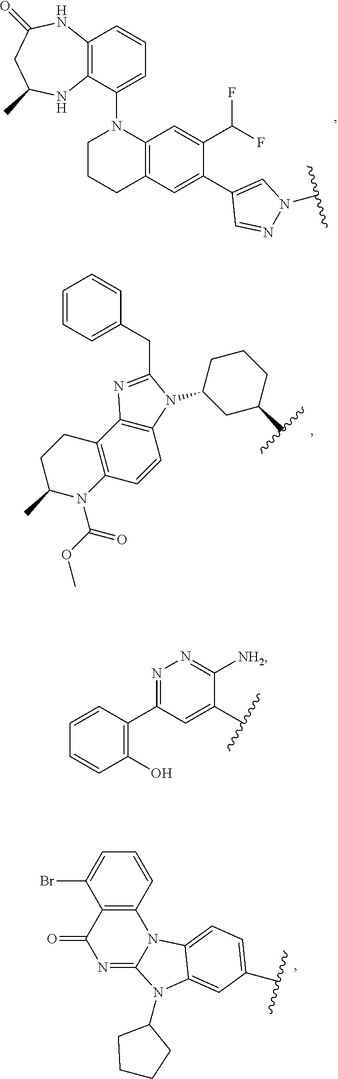 [SMILES: *n1cc(-c2cc3c(cc2C(F)F)N(c2cccc4c2N[C@@H](C)CC(=O)N4)CCC3)cn1 ; *[C@@H]1CCC[C@@H](n2c(Cc3ccccc3)nc3c4c(ccc32)N(C(=O)OC)[C@@H](C)CC4)C1 ; *c1cc(-c2ccccc2O)nnc1N ; *c1ccc2c(c1)n(C1CCCC1)c1nc(=O)c3c(Br)cccc3n21]

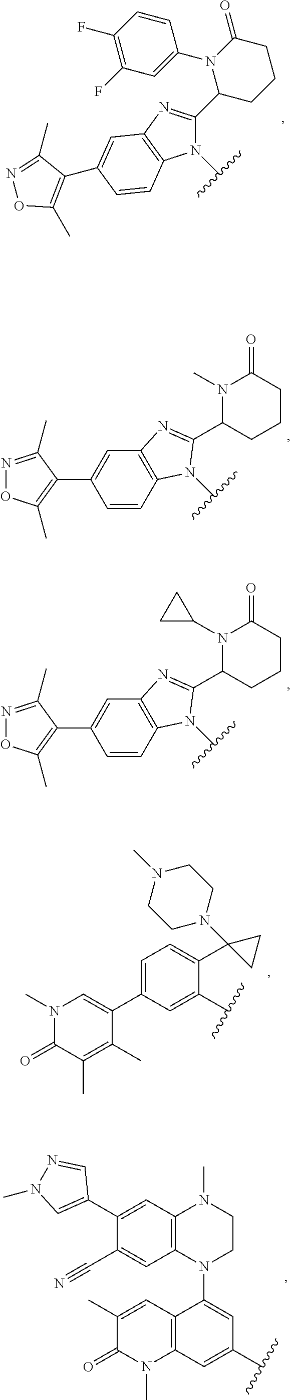 [SMILES: *n1c(C2CCCC(=O)N2c2ccc(F)c(F)c2)nc2cc(-c3c(C)noc3C)ccc21 ; *n1c(C2CCCC(=O)N2C)nc2cc(-c3c(C)noc3C)ccc21 ; *n1c(C2CCCC(=O)N2C2CC2)nc2cc(-c3c(C)noc3C)ccc21 ; *c1cc(-c2cn(C)c(=O)c(C)c2C)ccc1C1(N2CCN(C)CC2)CC1 ; *c1cc(N2CCN(C)c3cc(-c4cnn(C)c4)c(C#N)cc32)c2cc(C)c(=O)n(C)c2c1]

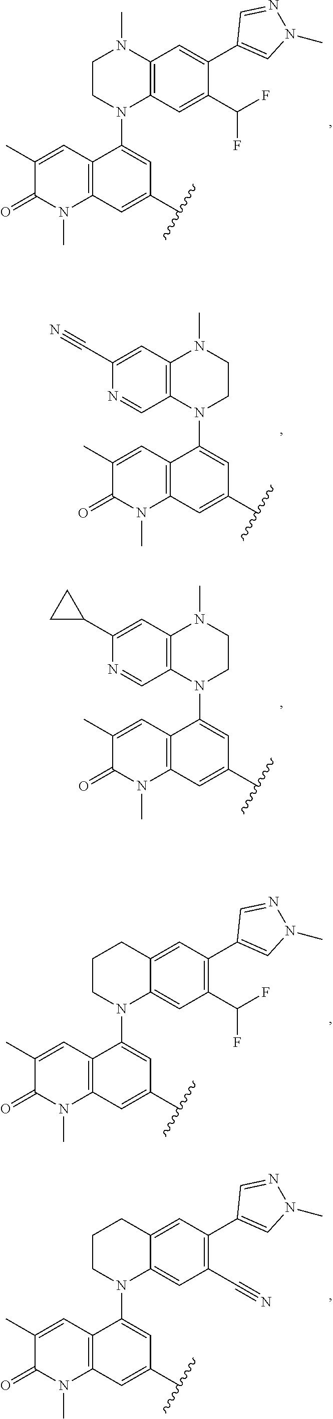 [SMILES: *c1cc(N2CCN(C)c3cc(-c4cnn(C)c4)c(C(F)F)cc32)c2cc(C)c(=O)n(C)c2c1 ; *c1cc(N2CCN(C)c3cc(C#N)ncc32)c2cc(C)c(=O)n(C)c2c1 ; *c1cc(N2CCN(C)c3cc(C4CC4)ncc32)c2cc(C)c(=O)n(C)c2c1 ; *c1cc(N2CCCc3cc(-c4cnn(C)c4)c(C(F)F)cc32)c2cc(C)c(=O)n(C)c2c1 ; *c1cc(N2CCCc3cc(-c4cnn(C)c4)c(C#N)cc32)c2cc(C)c(=O)n(C)c2c1]

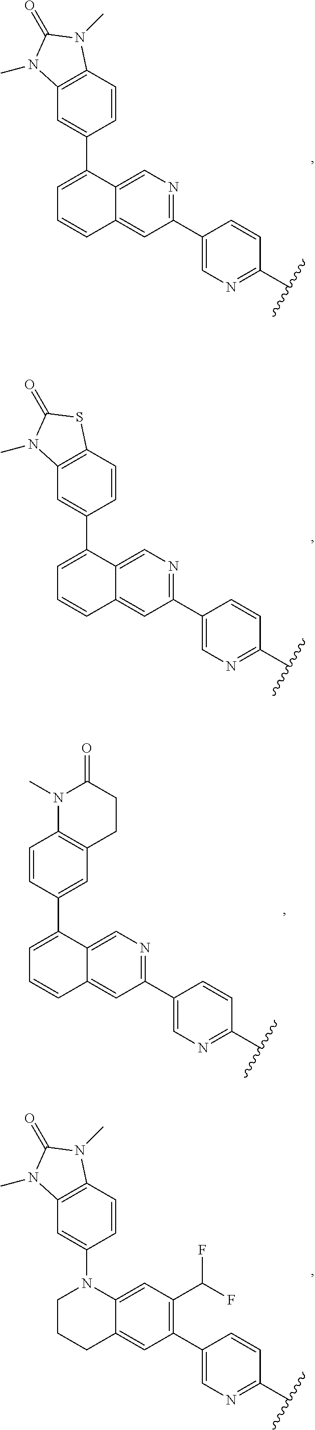 [SMILES: *c1ccc(-c2cc3cccc(-c4ccc5c(c4)n(C)c(=O)n5C)c3cn2)cn1 ; *c1ccc(-c2cc3cccc(-c4ccc5sc(=O)n(C)c5c4)c3cn2)cn1 ; *c1ccc(-c2cc3cccc(-c4ccc5c(c4)CCC(=O)N5C)c3cn2)cn1 ; *c1ccc(-c2cc3c(cc2C(F)F)N(c2ccc4c(c2)n(C)c(=O)n4C)CCC3)cn1]

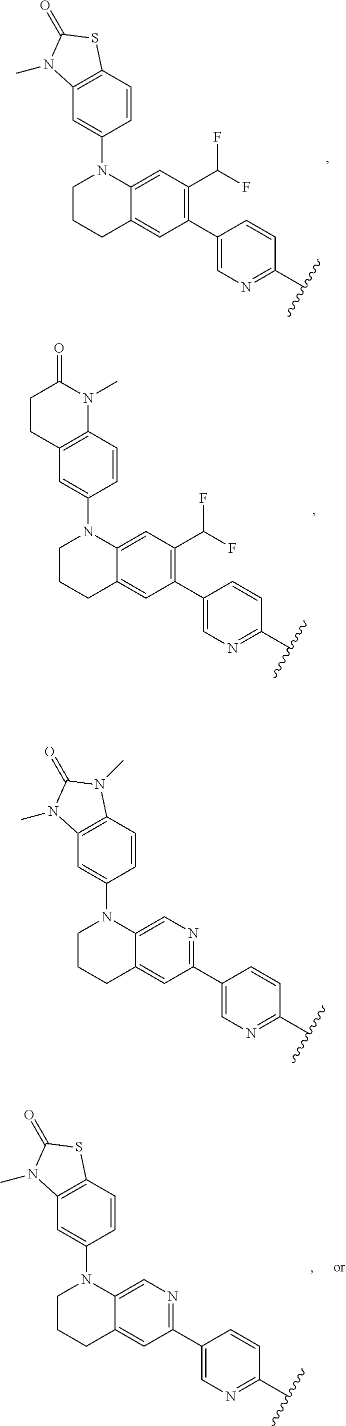 [SMILES: *c1ccc(-c2cc3c(cc2C(F)F)N(c2ccc4sc(=O)n(C)c4c2)CCC3)cn1 ; *c1ccc(-c2cc3c(cc2C(F)F)N(c2ccc4c(c2)CCC(=O)N4C)CCC3)cn1 ; *c1ccc(-c2cc3c(cn2)N(c2ccc4c(c2)n(C)c(=O)n4C)CCC3)cn1 ; *c1ccc(-c2cc3c(cn2)N(c2ccc4sc(=O)n(C)c4c2)CCC3)cn1]

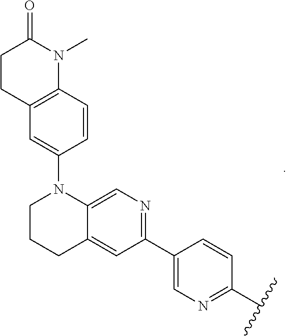 [SMILES: *c1ccc(-c2cc3c(cn2)N(c2ccc4c(c2)CCC(=O)N4C)CCC3)cn1]

57. The compound of any one of claims 9-14, wherein SB-CBP/p300 has one of the following structures: 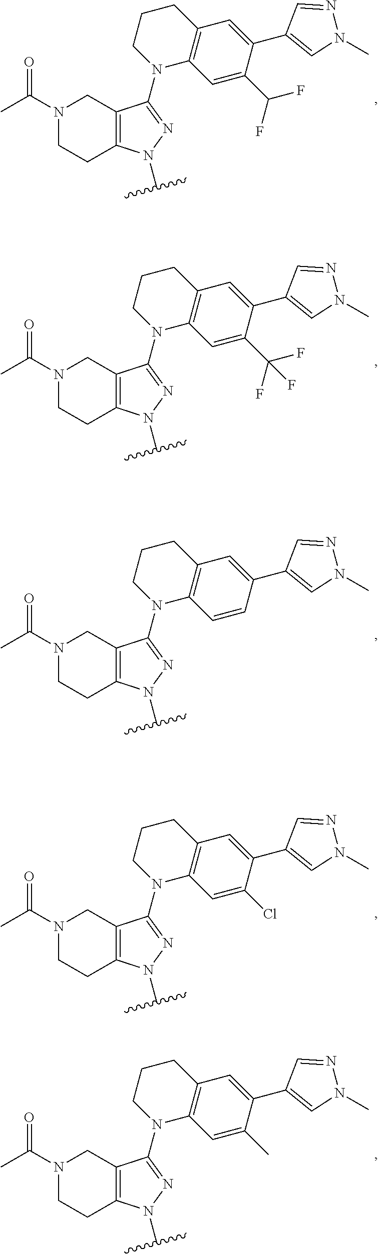 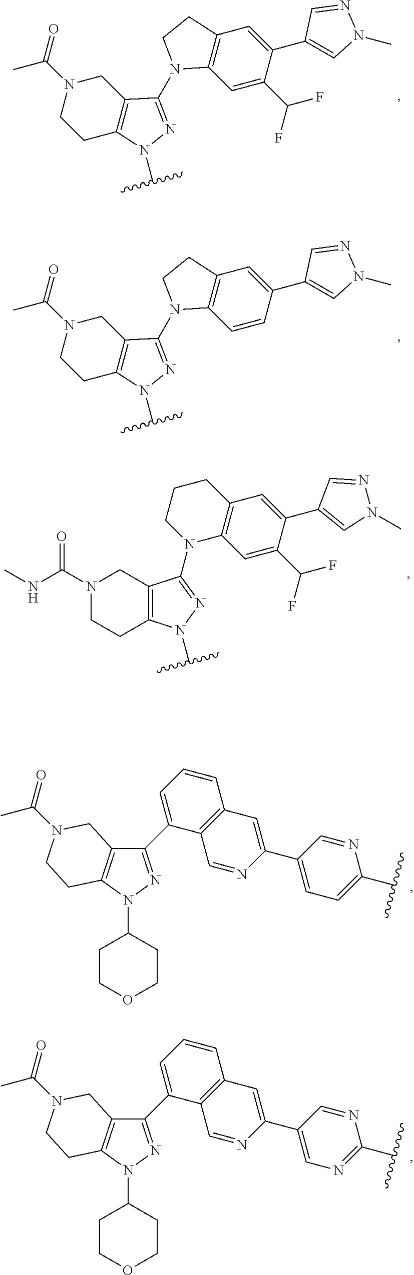 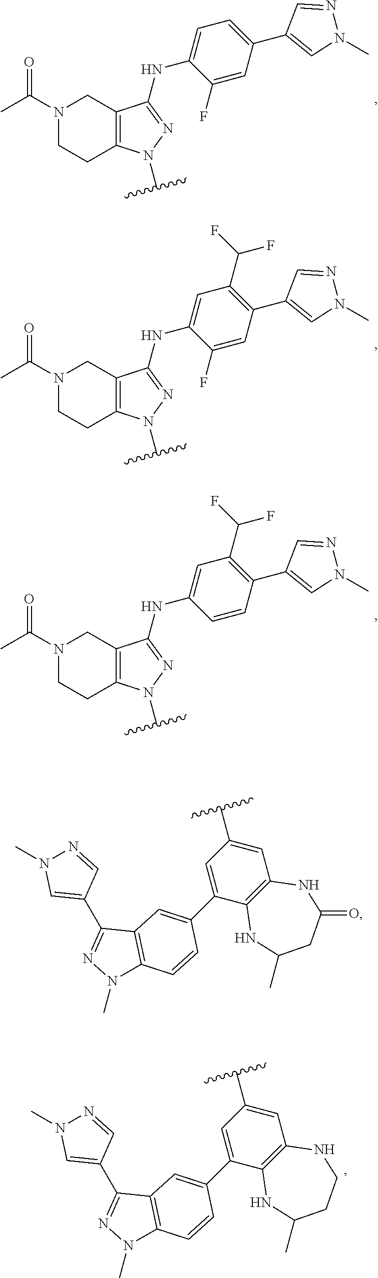 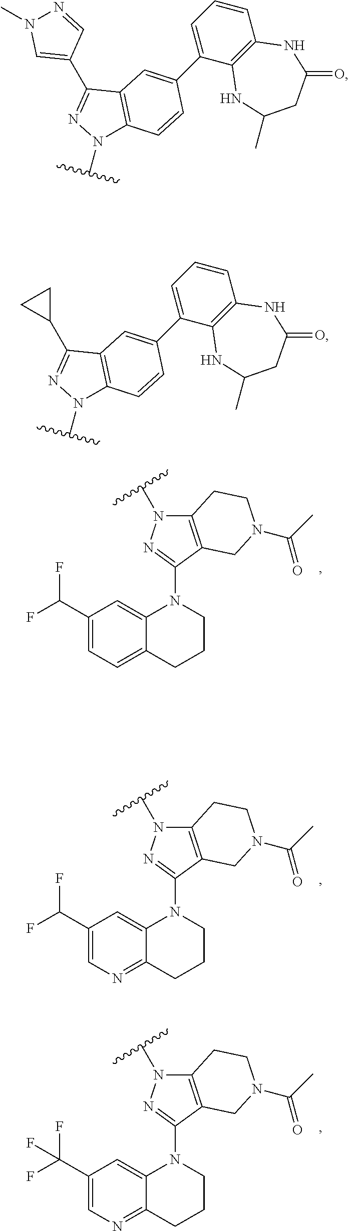 [SMILES: *n1nc(-c2cnn(C)c2)c2cc(-c3cccc4c3NC(C)CC(=O)N4)ccc21 ; *n1nc(C2CC2)c2cc(-c3cccc4c3NC(C)CC(=O)N4)ccc21 ; *n1nc(N2CCCc3ccc(C(F)F)cc32)c2c1CCN(C(C)=O)C2 ; *n1nc(N2CCCc3ncc(C(F)F)cc32)c2c1CCN(C(C)=O)C2 ; *n1nc(N2CCCc3ncc(C(F)(F)F)cc32)c2c1CCN(C(C)=O)C2] 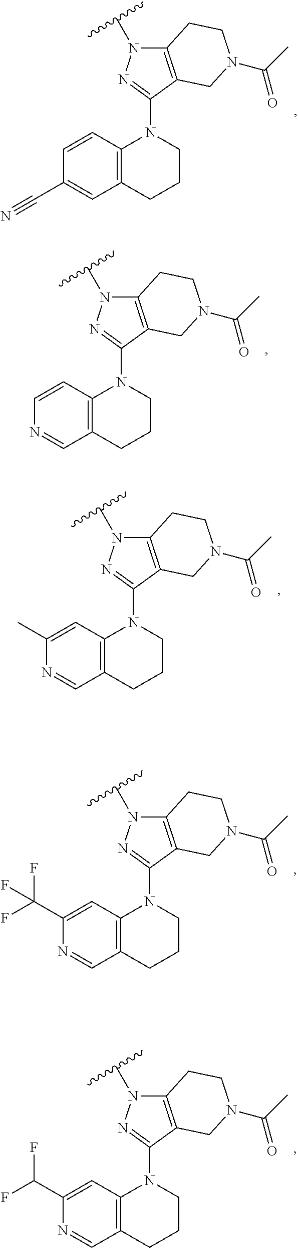 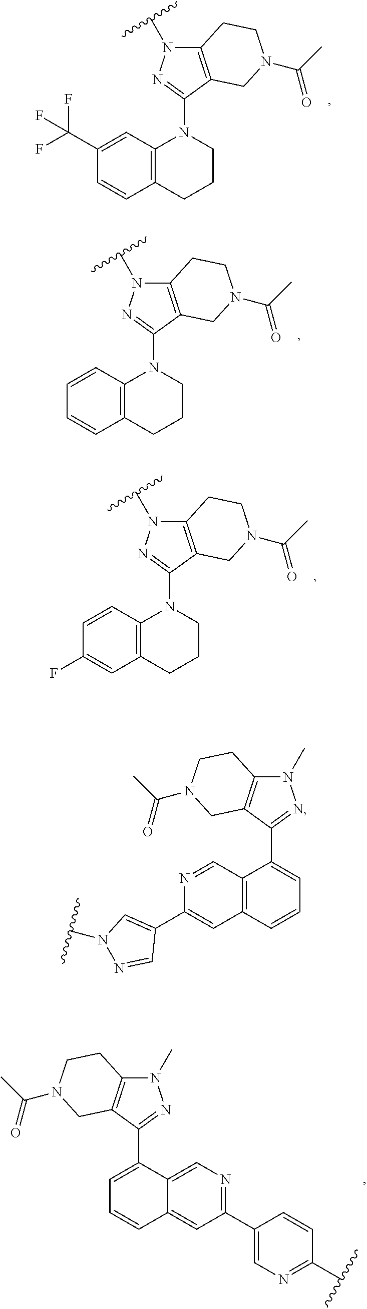 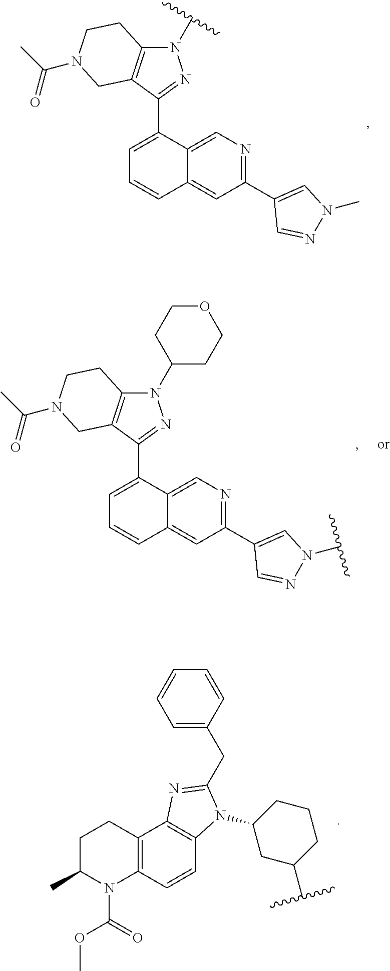
or a pharmaceutically acceptable salt or solvate thereof.

58. The compound of any one of claims 9-14, or a pharmaceutically acceptable salt or solvate thereof, wherein SB-CBP/p300 has one of the following structures:

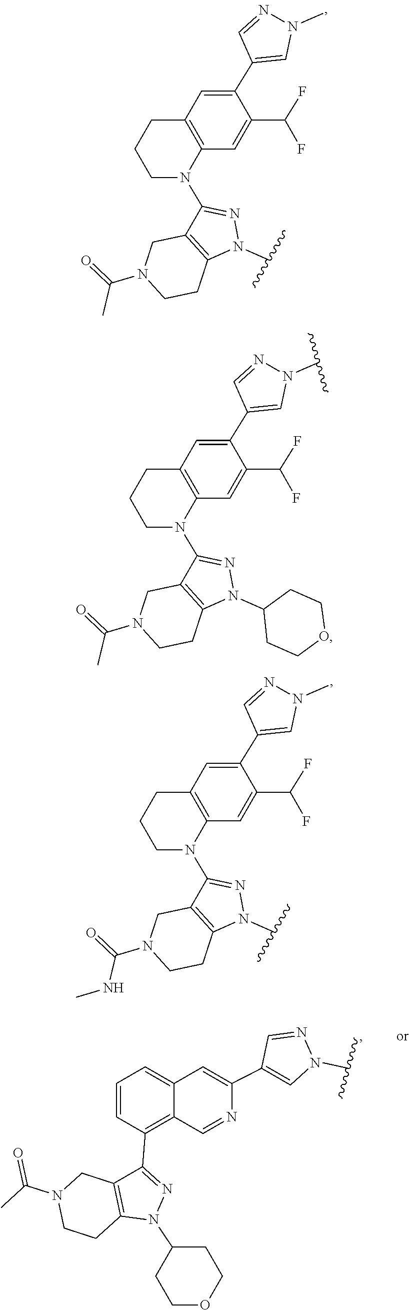

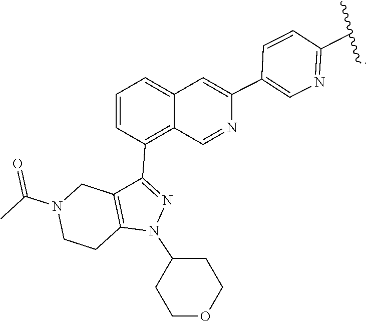 [SMILES: *c1ccc(-c2cc3cccc(-c4nn(C5CCOCC5)c5c4CN(C(C)=O)CC5)c3cn2)cn1]

59. The compound of any one of claims 9-14, or a pharmaceutically acceptable salt or solvate thereof, wherein SB-CBP/p300 has one of the following structures:

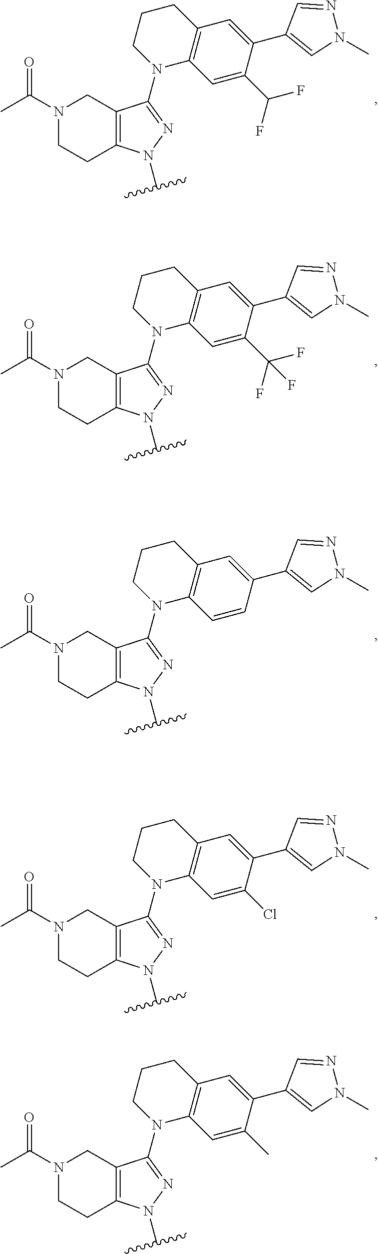

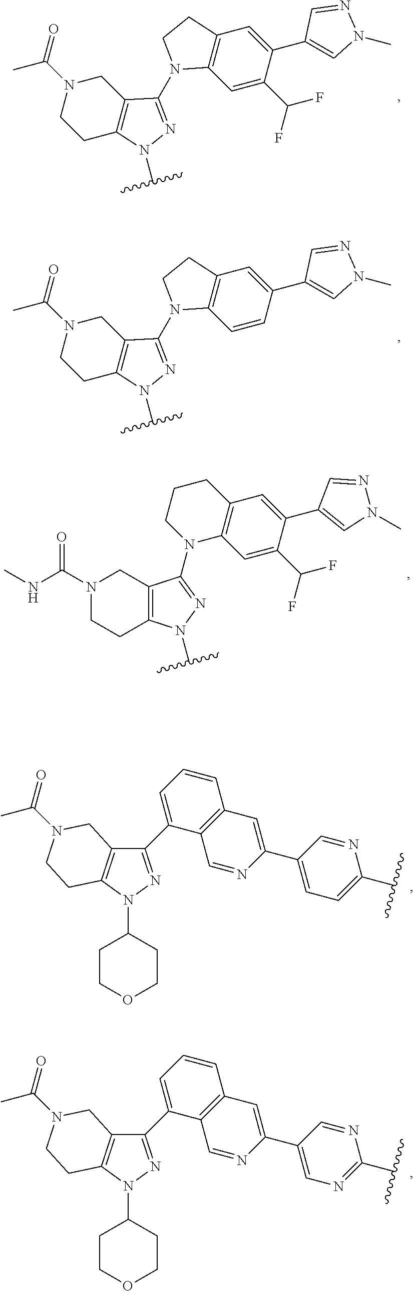

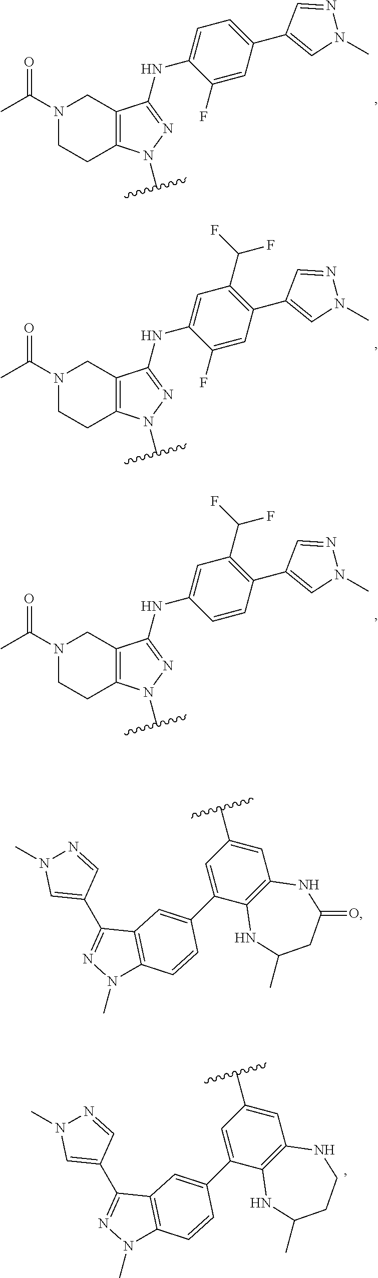

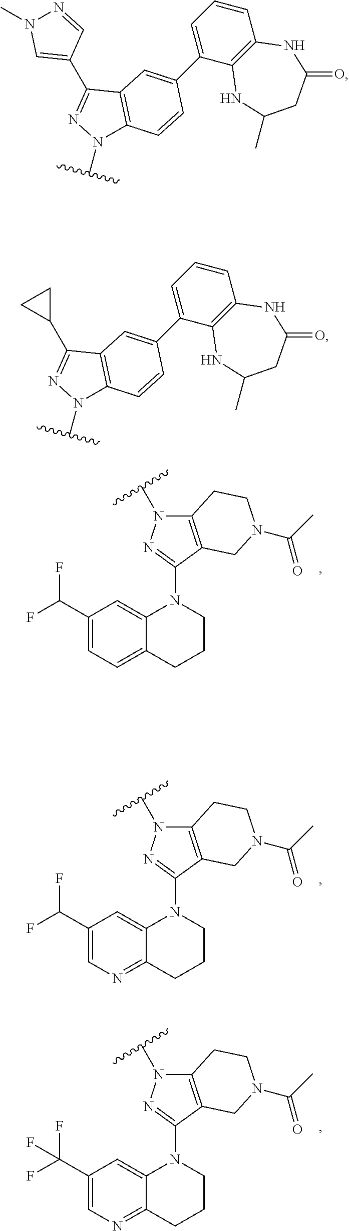 [SMILES: *n1nc(-c2cnn(C)c2)c2cc(-c3cccc4c3NC(C)CC(=O)N4)ccc21 ; *n1nc(C2CC2)c2cc(-c3cccc4c3NC(C)CC(=O)N4)ccc21 ; *n1nc(N2CCCc3ccc(C(F)F)cc32)c2c1CCN(C(C)=O)C2 ; *n1nc(N2CCCc3ncc(C(F)F)cc32)c2c1CCN(C(C)=O)C2 ; *n1nc(N2CCCc3ncc(C(F)(F)F)cc32)c2c1CCN(C(C)=O)C2]

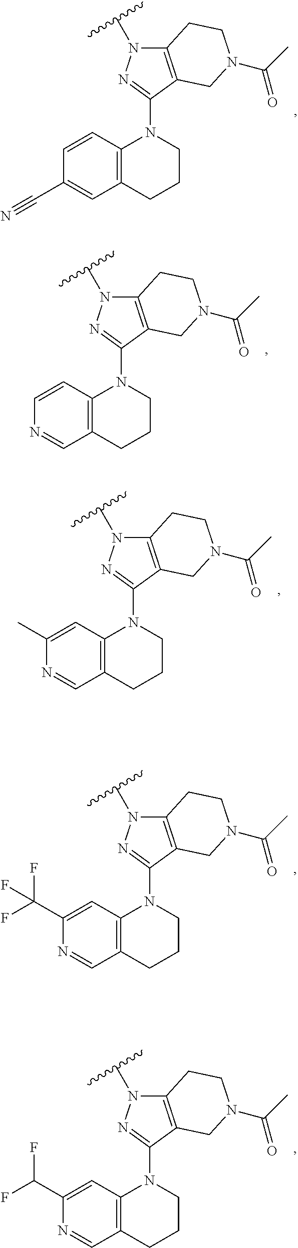

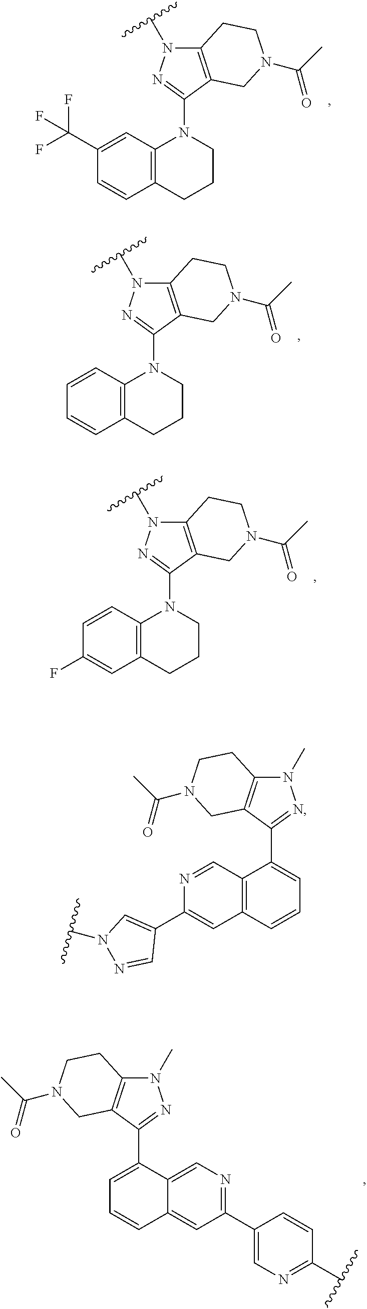

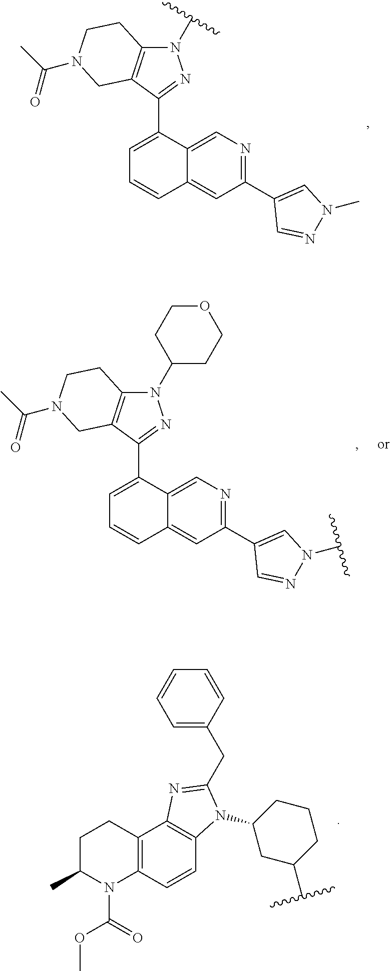

60. The compound of any one of claims 1-59, wherein the DP is a nuclear nuclear receptor (NR) transcription factor.

61. The compound of any one of claims 1-59, wherein the DP is a nuclear nuclear receptor (NR) transcription factor that is the androgen receptor (AR) and the BDP is a binder of AR (B-AR); or the DP is the estrogen receptor (ER) and the BDP is a binder of ER (B-ER); or retinoic acid receptor-related orphan nuclear receptor γ (RORγ) and the B-DP is a binder of RORγ (B-ROR).

62. The compound of any one of claims 1-59, wherein the DP is the androgen receptor (AR) and the BDP is a binder of AR (B-AR), wherein the B-AR is an AR antagonist, a selective androgen receptor modulator (SARM), or selective androgen receptor degrader (SARD); and the B-ER is an ER antagonist, a selective estrogen receptor modulator (SERM), or a selective estrogen receptor degrader (SERD);
wherein the AR antagonist, SARM, or SARD is a non-steroidal AR ligand or a steroidal AR ligand;
and wherein the ER antagonist, SERM, or SERD is a non-steroidal ER ligand or a steroidal ER ligand.

63. The compound of any one of claims 1-59, wherein the DP is the androgen receptor (AR) and the BDP is a binder of AR (B-AR) that is an AR antagonist, a selective androgen receptor modulator (SARM), or a selective androgen receptor degrader (SARD);
wherein the AR antagonist, SARM, or SARD binds to the ligand-binding domain (LBD) of AR.

64. The compound of claim 63, wherein the AR antagonist, SARM, or SARD comprises:
a head moiety that occupies the LBD region of the AR; and 1) an optional core moiety; 2) an optional tail moiety covalently attached to the core moiety; or 3) both 1) and 2).

65. The compound of claim 64, wherein the head moiety of the AR antagonist, SARM, or SARD forms hydrogen bonds with the side chains of Gln 711 and Arg752 of the LBD of AR.

66. The compound of any one of claims 64-65, wherein:
the optional linker is covalently attached to the head moiety if the optional core moiety and optional tail moiety are absent; or
the optional linker is covalently attached to the core moiety if the optional tail moiety is absent; or
the optional linker is covalently attached to the tail moiety.

67. The compound of claim 63, wherein B-AR is flutamide, hydroxylflutamide, nilutamide, bicalutamide, enzalutamide, apalutamide, ODM201, AZD3514, or BMS641988.

68. The compound of any one of claims 64-66, wherein the head group is selected from: 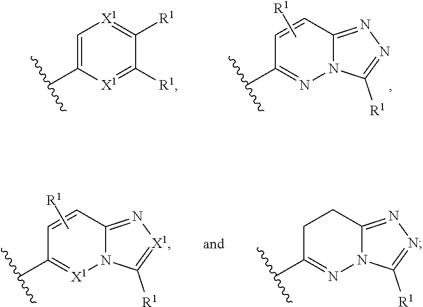
each X¹ is independently —CR¹— or —N—;
each R¹ is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted 3- to 8-membered heterocycloalkyl, —CN, —NO₂, —OH, —OR⁴, OC(═O)R⁴, —OC(═O)N(R⁵)₂, —OC(═O)OR⁵, —OC(═O)NR⁵, —SH, —SR⁴, —S(═O)R⁴, —S(═O)₂R⁵, —S(═O)₂OR⁴, —S(═O)₂N(R⁵)₂, —N(R⁵)₂, —NR⁵C(═O)NR⁵, —NR⁵C(═O)R⁴, —NR⁵C(═O)OR⁵, —NR⁵S(═O)₂R⁵, —C(═O)R⁵, —C(═O)OR⁵, or —C(═O)N(R⁵)₂.

69. The compound of any one of claims 64-66, wherein the head group is selected from: 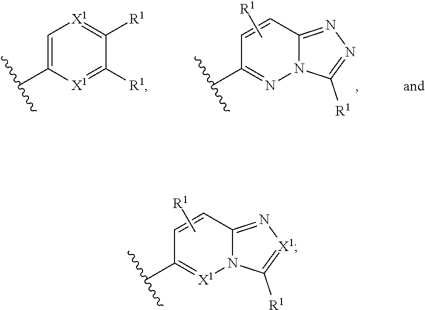
one X¹ is —CR¹— and the other X¹ is —CR¹— or —N—;
each R¹ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, —OCH₂CH₃, —CN, —C(═O)NH₂ or —C(═O)NH(CH₃).

70. The compound of any one of claims 64-66, wherein the head group is selected from: 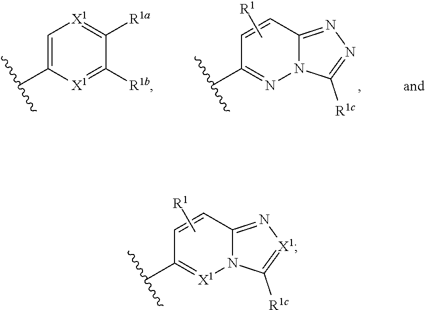
each X is independently —CR¹— or —N—;
R^{1a} is —CN, —NO₂, —C(═O)R⁵, —C(═O)OR⁵, or —C(═O)N(R⁵)₂;
R^{1b} is hydrogen, halogen, C₁-C₄alkyl, C₁-C₄fluoroalkyl, —CN, —OH, —OR⁴, or —SR₄;
R^{1c} is hydrogen, halogen, C₁-C₄alkyl, C₁-C₄fluoroalkyl, or —CN;
each R¹ is independently hydrogen, halogen, C₁-C₄alkyl, C₁-C₄fluoroalkyl, —CN, —OH, or —OR⁴;
each R⁴ is independently C₁-C₄alkyl, or C₁-C₄fluoroalkyl;
each R⁵ is independently hydrogen, C₁-C₄alkyl, or C₁-C₄fluoroalkyl.

71. The compound of any one of claims 64-66, wherein the head group is selected from: 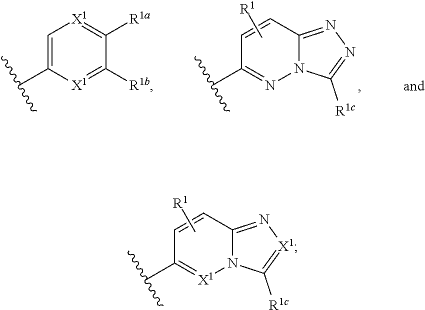
one X¹ is —CR¹— and the other X¹ is —CR¹— or —N—;
R^{1a} is —CN, —NO₂, —C(═O)NH₂ or —C(═O)NH(CH₃);
R^{1b} is hydrogen, F, Cl, Br, I, —CH₃, —CH₂CH₃, —CH₂F, —CHF₂, —CF₃, —OH, —OCF₃, —OCH₃, —OCH₂CH₃, or —CN;
each R^{1c} is hydrogen, F, Cl, Br, —CH₃, —CH₂F, —CHF₂, or —CF₃;
each R¹ is independently hydrogen, F, Cl, Br, I, —CH₃, —CH₂CH₃, —CH₂F, —CHF₂, —CF₃, —CH₂CH₂F, —CH₂CHF₂, —CH₂CF₃, —OH, —OCF₃, —OCH₃, or —OCH₂CH₃.

72. The compound of any one of claims 64-66, wherein the head group is selected from:

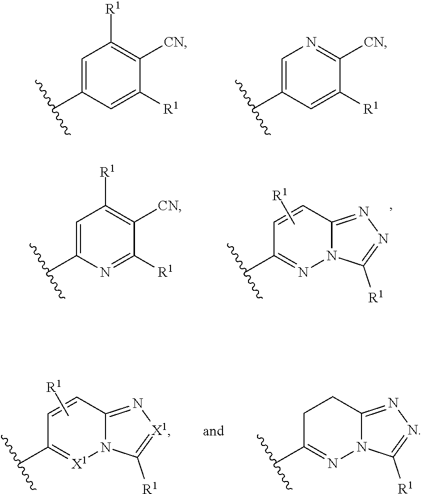

73. The compound of any one of claims 64-66, wherein the head group is selected from:

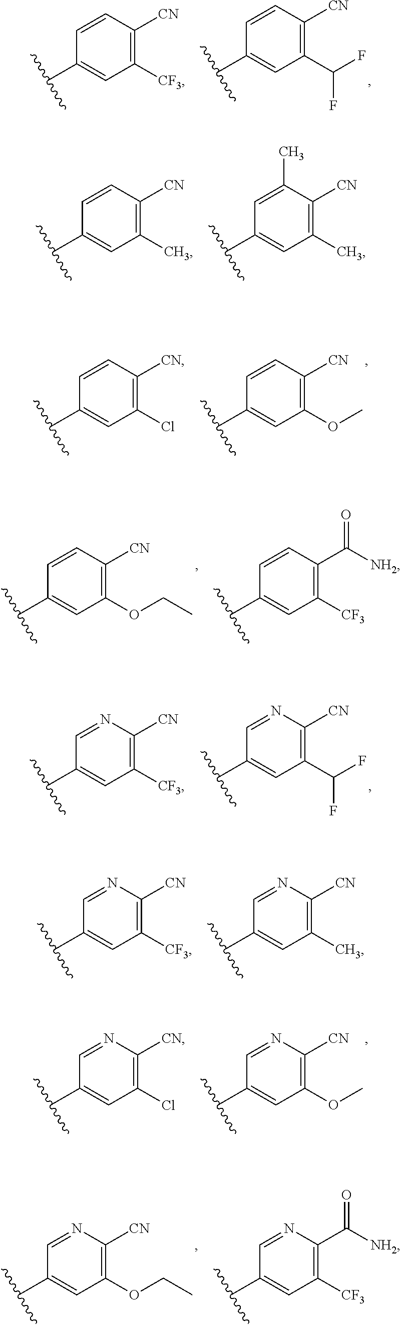

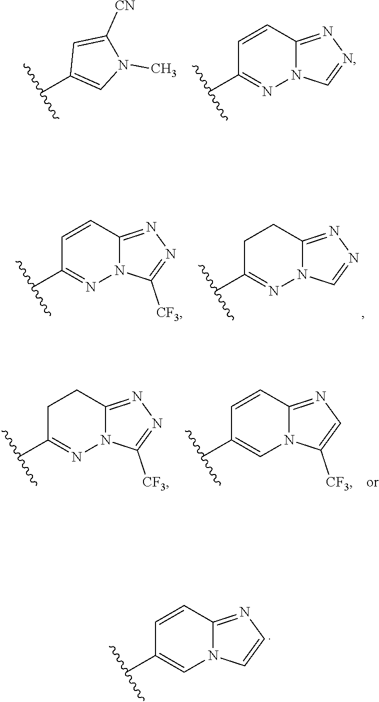

74. The compound of any one of claims 64-66 or 68-73, wherein the optional core comprises a group selected from: 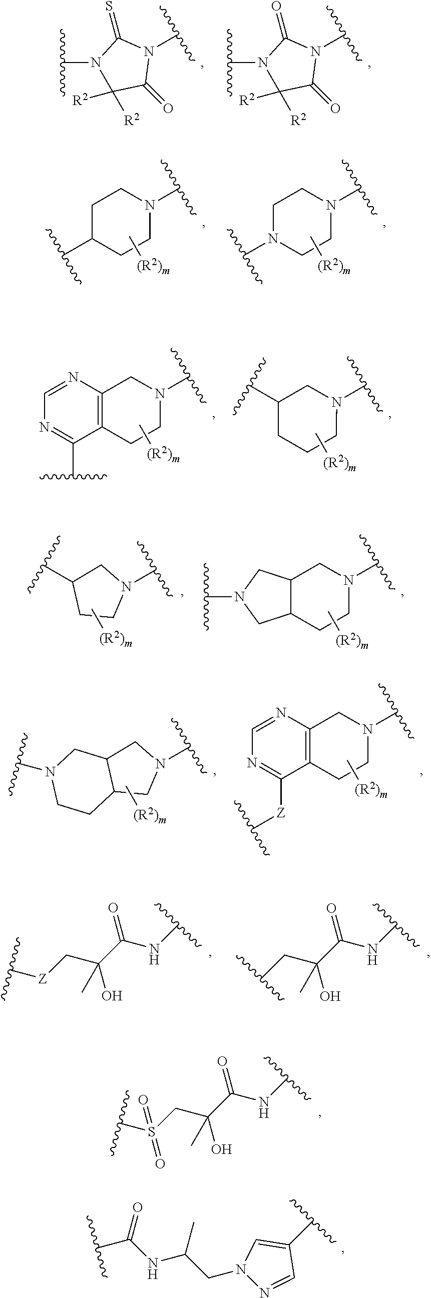 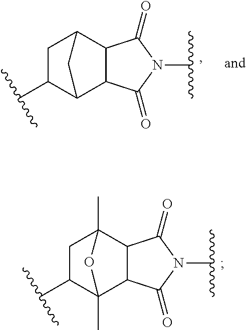 [SMILES: *C1CC2CC1C1C(=O)N(*)C(=O)C21 ; *C1CC2(C)OC1(C)C1C(=O)N(*)C(=O)C12]
Z is —O— or —NR⁵—;
each R² is independently hydrogen halogen substituted or unsubstituted C₁-C₆alkyl, substituted or unsubstituted C₁-C₆fluoroalkyl, substituted or unsubstituted C₁-C₆heteroalkyl, —CN, —NO₂, —OH, —OR⁴, OC(═O)R⁴, —S(═O)R⁴, —S(═O)₂R⁵, —S(═O)₂N(R⁵)₂, —N(R⁵)₂, —NR⁵C(═O)NR⁵, —NR⁵C(═O)R⁴, —C(═O)R⁵, —C(═O)OR⁵, or —C(═O)N(R⁵)₂; or
two R² on the same carbon atom are taken together with the carbon atom to which they are attached to form a substituted or unsubstituted C₃-C₈cycloalkyl or a substituted or unsubstituted monocyclic 3- to 8-membered heterocycloalkyl;
m is 0, 1, 2, 3, or 4;
each s is independently 1, 2, or 3;
each R⁴ is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
each R⁴ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₆cycloalkyl, substituted or unsubstituted monocyclic 3- to 8-membered heterocycloalkyl, substituted or unsubstituted aryl, or substituted or unsubstituted monocyclic heteroaryl;
or two R⁵ on the same N atom are taken together with the N atom to which they are attached to form a substituted or unsubstituted N-containing heterocycle.

75. The compound of any one of claims 64-66 or 68-73, wherein the optional core and optional tail comprises a group selected from:

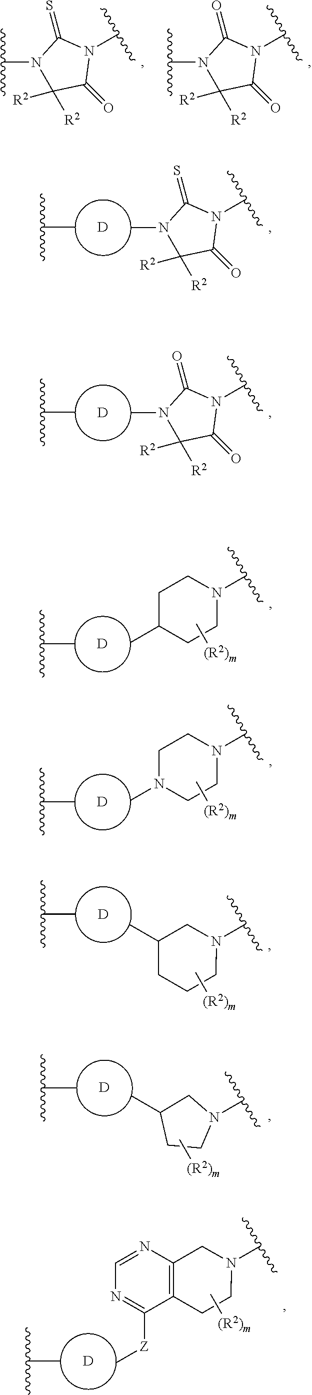

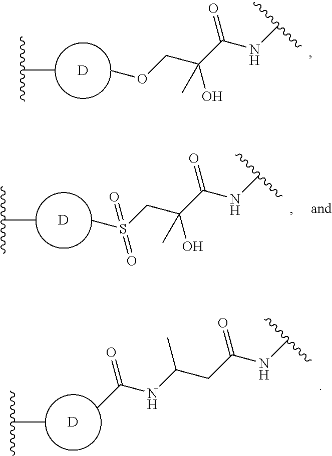

76. The compound of claim 75, wherein the optional tail comprises a ring D that is a 5-, 6-, 8-, 9- or 10-membered aryl or a 5-, 6-, 8-, 9- or 10-membered heteroaryl that is optionally substituted with s R³; each R³ is independently hydrogen, halogen, substituted or unsubstituted C₁-C₆alkyl, substituted or unsubstituted C₁-C₆fluoroalkyl, substituted or unsubstituted C₁-C₆heteroalkyl, —CN, —OH, —OR⁴, or —N(R⁵)₂.

77. The compound of any one of claims 75-76, wherein ring D is phenyl, naphthyl, pyridinyl, pyrimidinyl, pyridazinyl, pyrazinyl, triazinyl, pyrrolyl, thiazolyl, oxazolyl, isoxazolyl, oxadiazolyl, imidazolyl, pyrazolyl, isothiazolyl, triazolyl, and tetrazolyl, wherein each ring D is optionally substituted substituted with a R³.

78. The compound of any one of claims 75-77, wherein ring D is 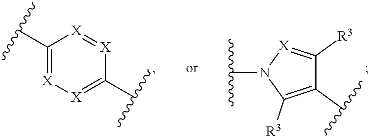
each X is independently —CR³— or —N—.

79. The compound of any one of claims 75-77, wherein ring D is

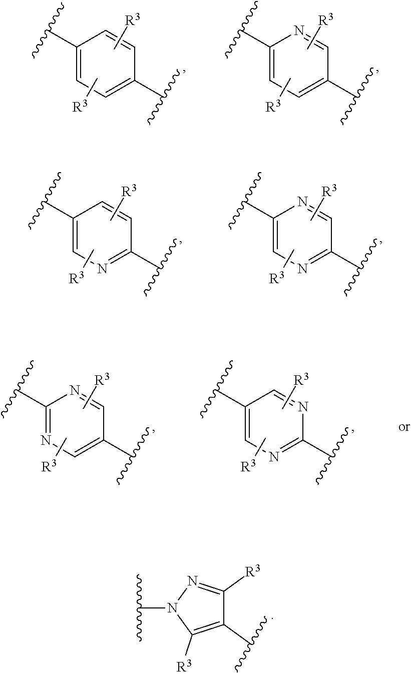

80. The compound of claim 62, wherein B-AR has one of the following structures: 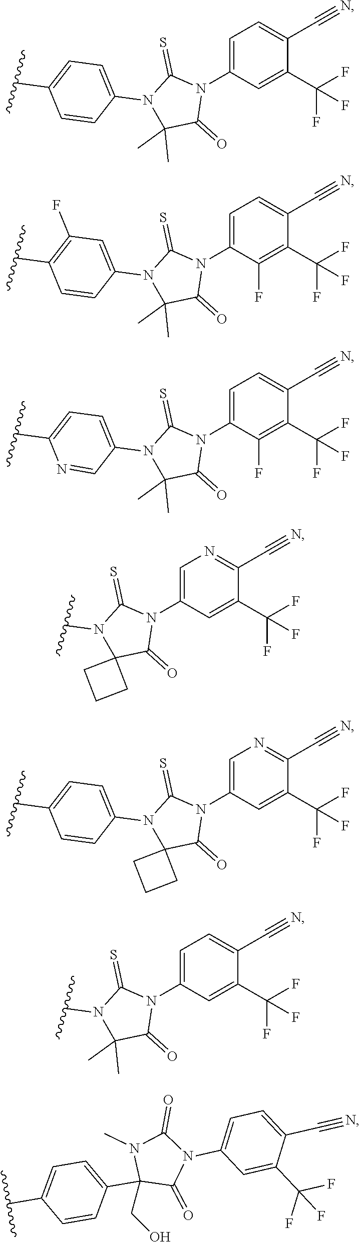 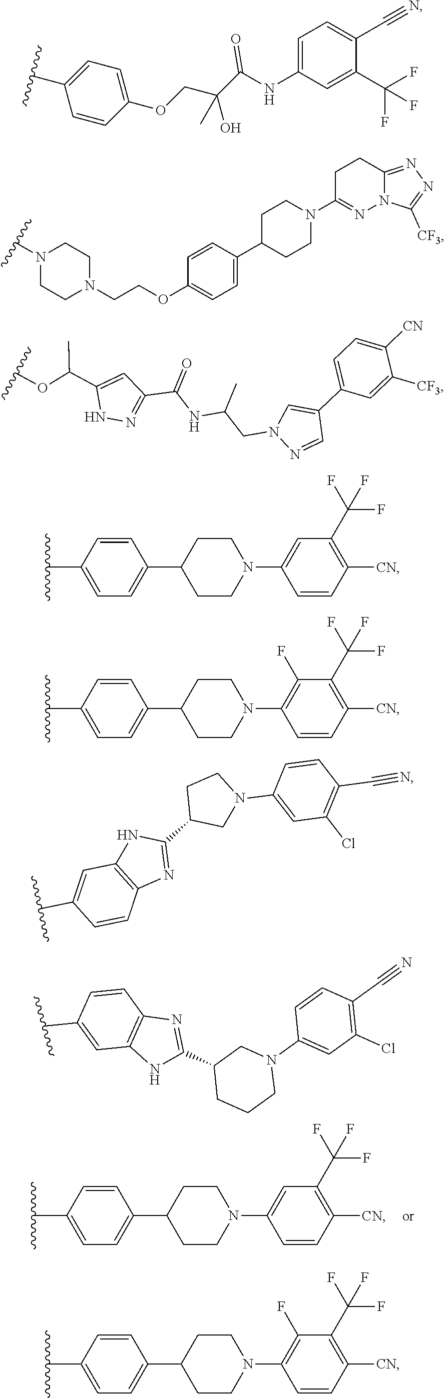
or a pharmaceutically acceptable salt or solvate thereof.

81. The compound of any one of claims 1-59, wherein the DP is the estrogen receptor (ER) and the BDP is a binder of ER (B-ER) that is an ER antagonist, a selective estrogen receptor modulator (SERM), or a selective estrogen receptor degrader (SERD).

82. The compound of claim 81, wherein the B-ER binds to the ligand-binding domain (LBD) of ER.

83. The compound of any one of claims 81-82, wherein the B-ER is: 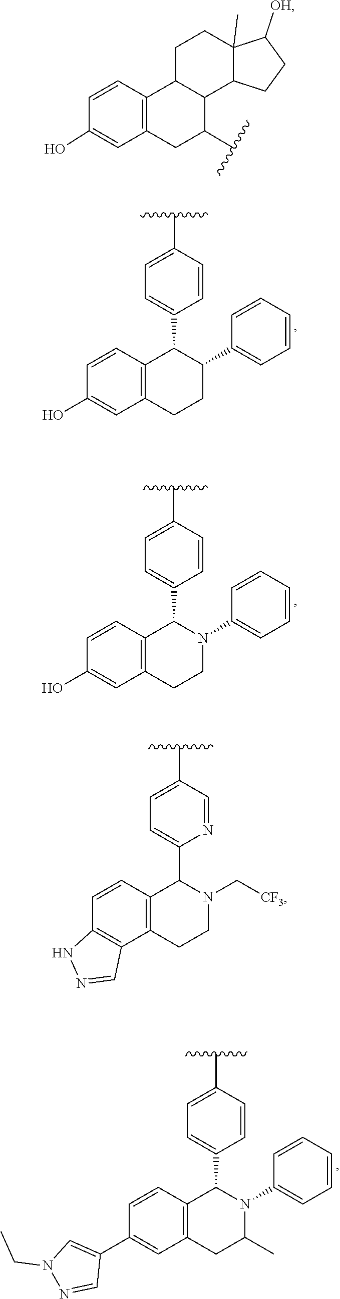 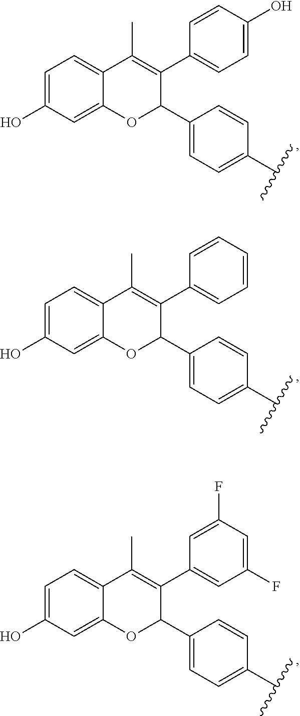 [SMILES: *c1ccc(C2Oc3cc(O)ccc3C(C)=C2c2ccc(O)cc2)cc1 ; *c1ccc(C2Oc3cc(O)ccc3C(C)=C2c2ccccc2)cc1 ; *c1ccc(C2Oc3cc(O)ccc3C(C)=C2c2cc(F)cc(F)c2)cc1] 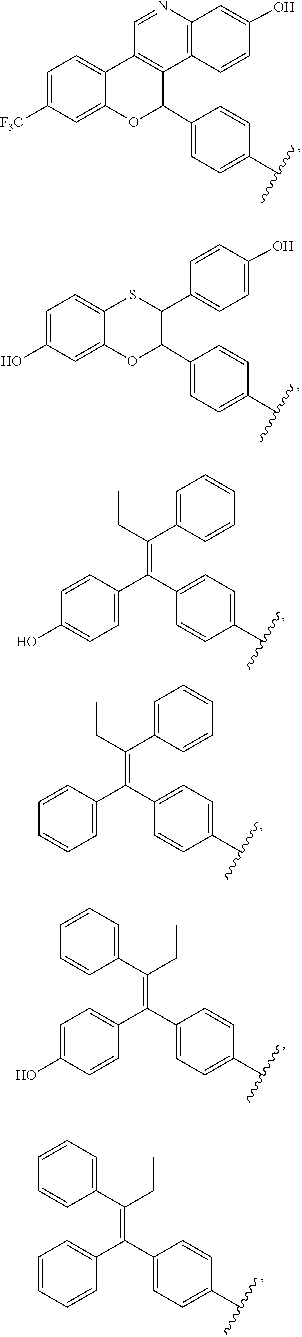 [SMILES: *c1ccc(C2Oc3cc(C(F)(F)F)ccc3-c3cnc4cc(O)ccc4c32)cc1 ; *c1ccc(C2Oc3cc(O)ccc3SC2c2ccc(O)cc2)cc1 ; *c1ccc(/C(=C(/CC)c2ccccc2)c2ccc(O)cc2)cc1 ; *c1ccc(/C(=C(/CC)c2ccccc2)c2ccccc2)cc1 ; *c1ccc(/C(=C(\CC)c2ccccc2)c2ccc(O)cc2)cc1 ; *c1ccc(/C(=C(\CC)c2ccccc2)c2ccccc2)cc1] 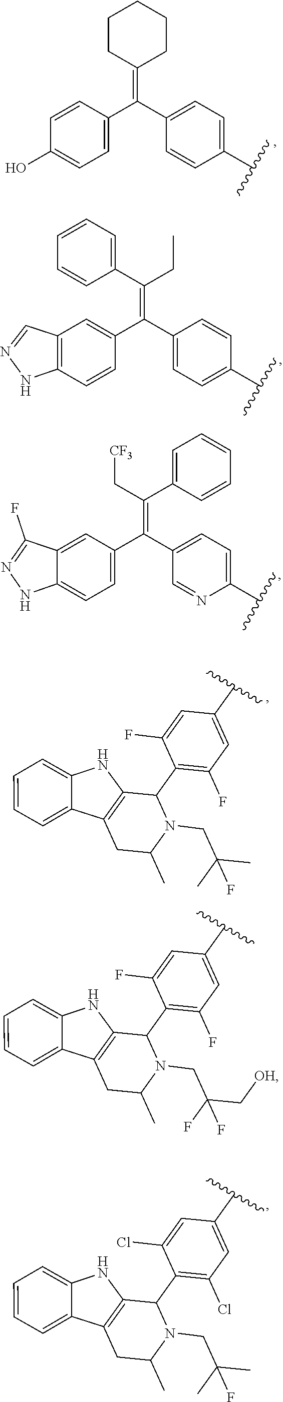 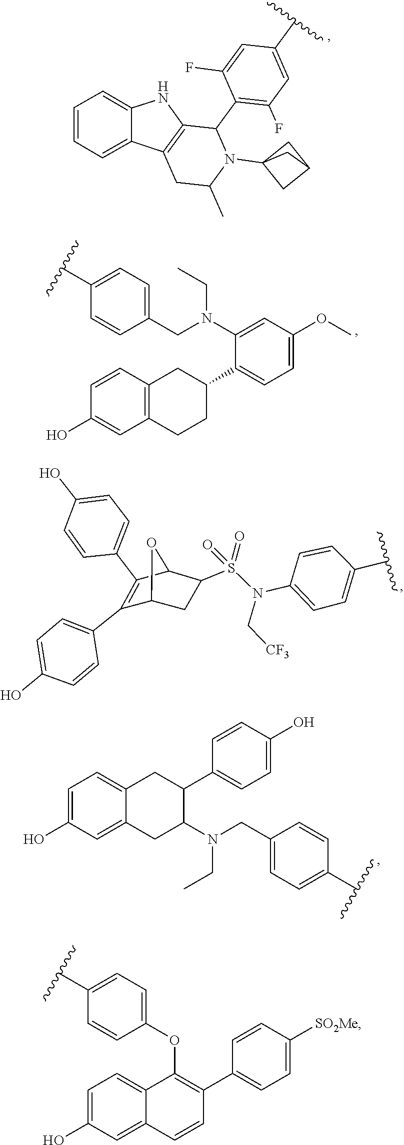 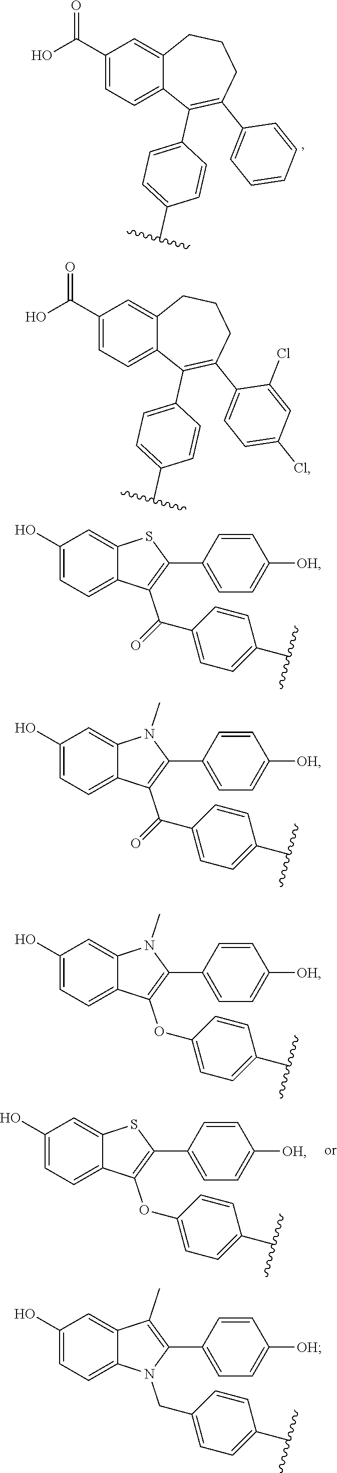
or a pharmaceutically acceptable salt or solvate thereof.

84. The compound of any one of claims 1-59, wherein the DP is chymase and the B-DP is a chymase inhibitor.

85. The compound of claim 84, wherein the chymase inhibitor is selective for chymase over cathepsin G.

86. The compound of claim 84 or 85, wherein the chymase inhibitor binds to Ser195 in the active cleft of chymase, binds to the S1 pocket of chymase, or both.

87. The compound of any one of claims 84-86, wherein the chymase inhibitor is HY-109059, Fulacimstat, BAY1142524, HY-12370, TY-51469, HY-100269, Chymase-IN-1, HY-12514, NK3201, NK3201, HY-122161, JNJ-10311795, RWJ-355871, JNJ-10311795 (RWJ-355871), HY-126988, TPC-806, SP-Chymostatin B, or analog thereof.

88. The compound of any one of claims 84-86, wherein the chymase inhibitor has one of the following structures:

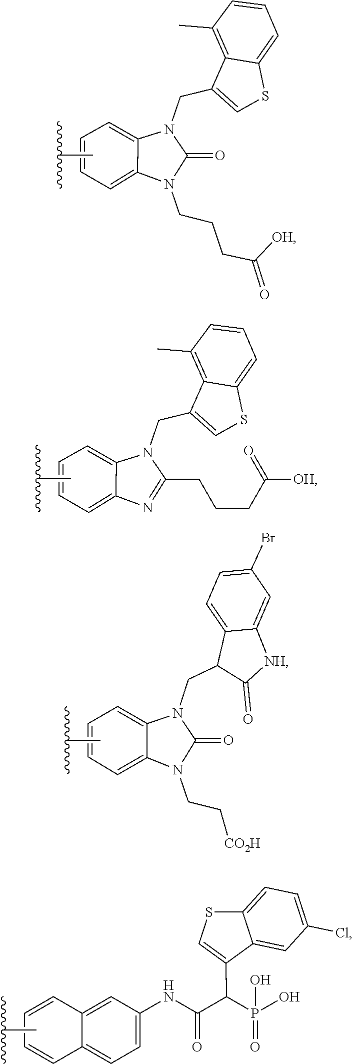

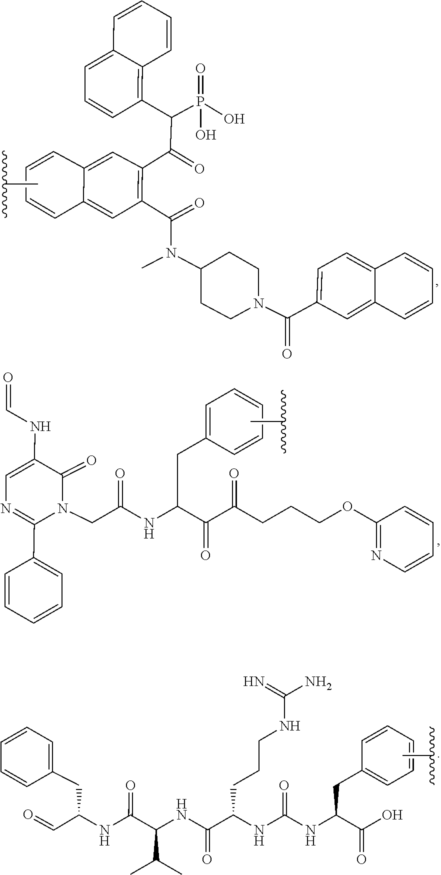

89. The compound of any one of claims 1-59, wherein the DP is retinoic acid receptor-related orphan nuclear receptor γ (RORγ) and the B-DP is a binder of RORγ (B-ROR).

90. The compound of claim 89, wherein the B-ROR is a RORγ antagonist, RORγ inverse agonist, RORγ inhibitor, or RORγ agonist.

91. The compound of claim 89 or 90, wherein the B-ROR binds to the ligand-binding domain (LBD) of RORγ.

92. The compound of any one of claims 89-91, wherein the B-ROR is S18-000003, A213, SHR168442, TMP778, BMS-986251, BMS-986313, A-9758, Digoxin, FC99, JNJ-61803534, SR2211, Ursolic acid, SR1001, TAK-828F, JNJ-61803534, JTE-451 (Retezorogant), BI 730357 (Bevurogant), ABBV-157 (Cedirogant), RTA-1701, AUR101, GSK2981278, PF-06763809, VTP-43742 (Vimirogant), AZD0284, GSK805, VPR-254, BIl19, CQMU152, BIX119, VTP-938, or analog thereof.

93. The compound of any one of claims 89-91, wherein the B-ROR has one of the following structures:

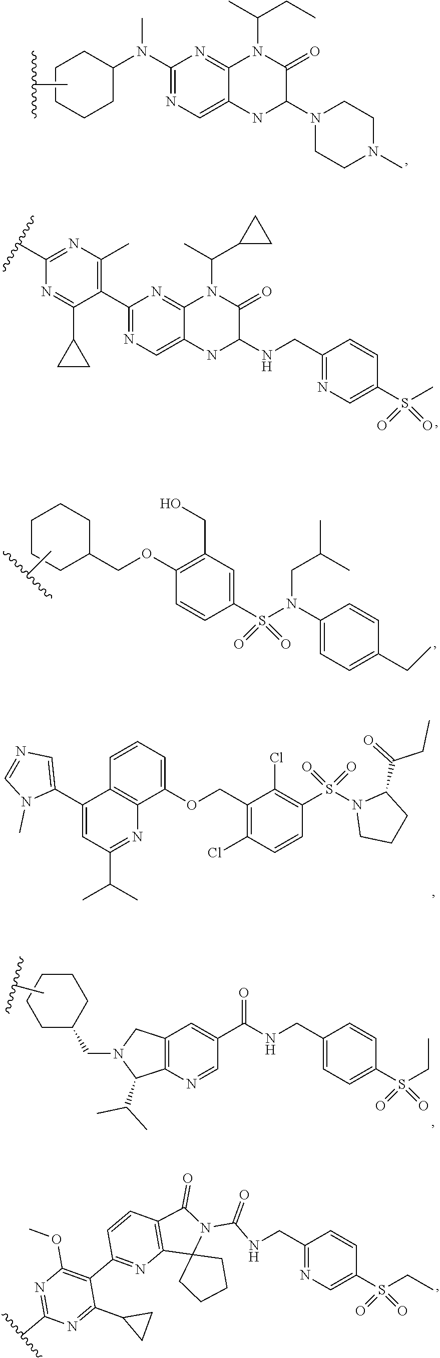

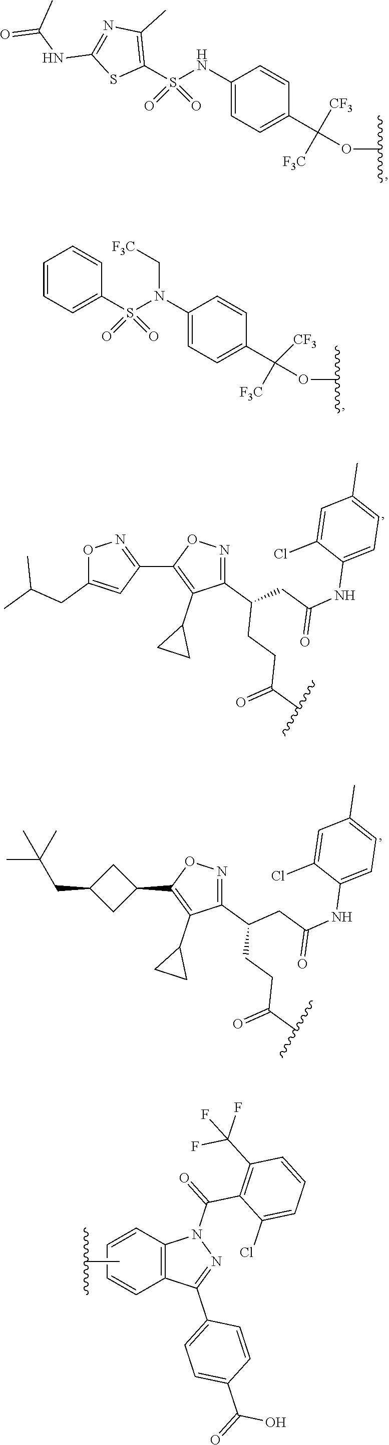

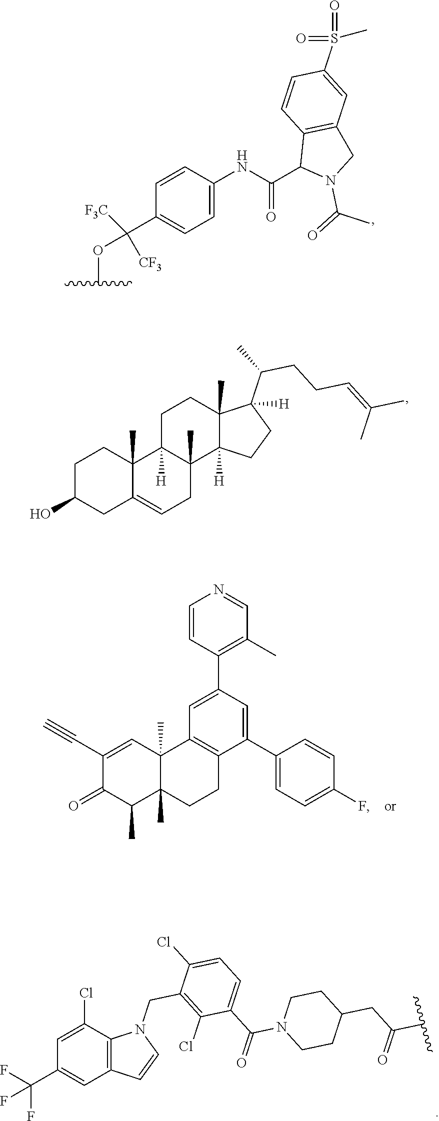

94. The compound of any one of claims 1-59, wherein the DP is indoleamine 2,3-dioxygenase 1 (IDO1) and the B-DP is a IDO1 inhibitor.

95. The compound of claim 94, wherein the B-DP is a IDO1 inhibitor that has one of the following structures:

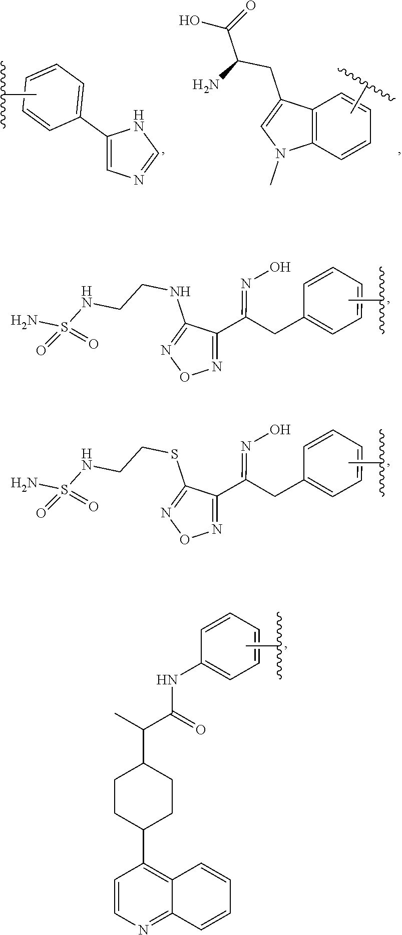

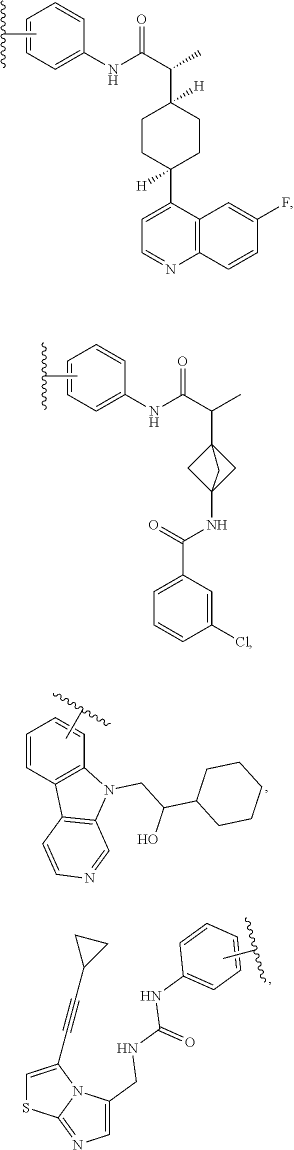

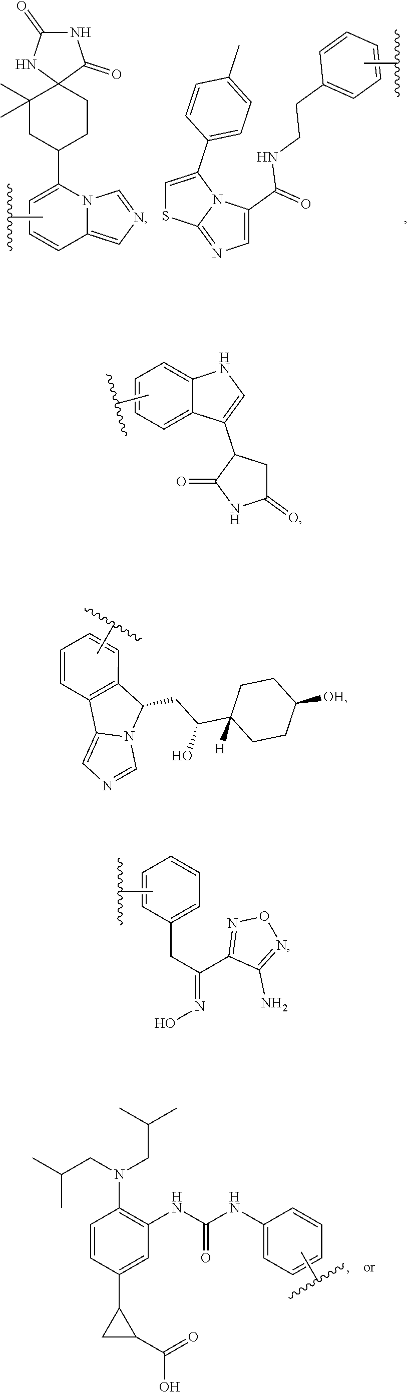

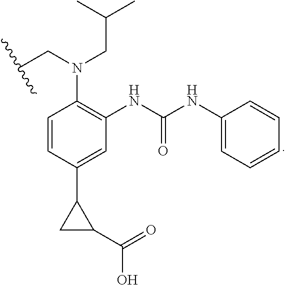 [SMILES: *CN(CC(C)C)c1ccc(C2CC2C(=O)O)cc1NC(=O)Nc1ccccc1]

96. The compound of any one of claims 1-95, or a pharmaceutically acceptable salt or solvate thereof, wherein L is absent.

97. The compound of any one of claims 1-95, or a pharmaceutically acceptable salt or solvate thereof, wherein L comprises substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted 3- to 10-membered heterocycloalkyl, substituted or unsubstituted aryl, substituted or unsubstituted heteroaryl, or combinations thereof.

98. The compound of any one of claims 1-78, or a pharmaceutically acceptable salt or solvate thereof, wherein L is absent or 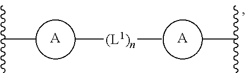
wherein:
each A is independently absent, substituted or unsubstituted monocyclic C₃-C₁₀cycloalkyl, substituted or unsubstituted bridged bicyclic C₃-C₁₀cycloalkyl, substituted or unsubstituted fused bicyclic C₃-C₁₀cycloalkyl, substituted or unsubstituted spiro bicyclic C₃-C₁₀cycloalkyl, substituted or unsubstituted monocyclic 3- to 10-membered heterocycloalkyl, substituted or unsubstituted bridged bicyclic 3- to 10-membered heterocycloalkyl, substituted or unsubstituted fused bicyclic 3- to 10-membered heterocycloalkyl, substituted or unsubstituted spiro bicyclic 3- to 10-membered heterocycloalkyl, substituted or unsubstituted aryl, or substituted or unsubstituted heteroaryl, wherein each A is independently unsubstituted or substituted with x R^{2b};
each L¹ is independently absent, 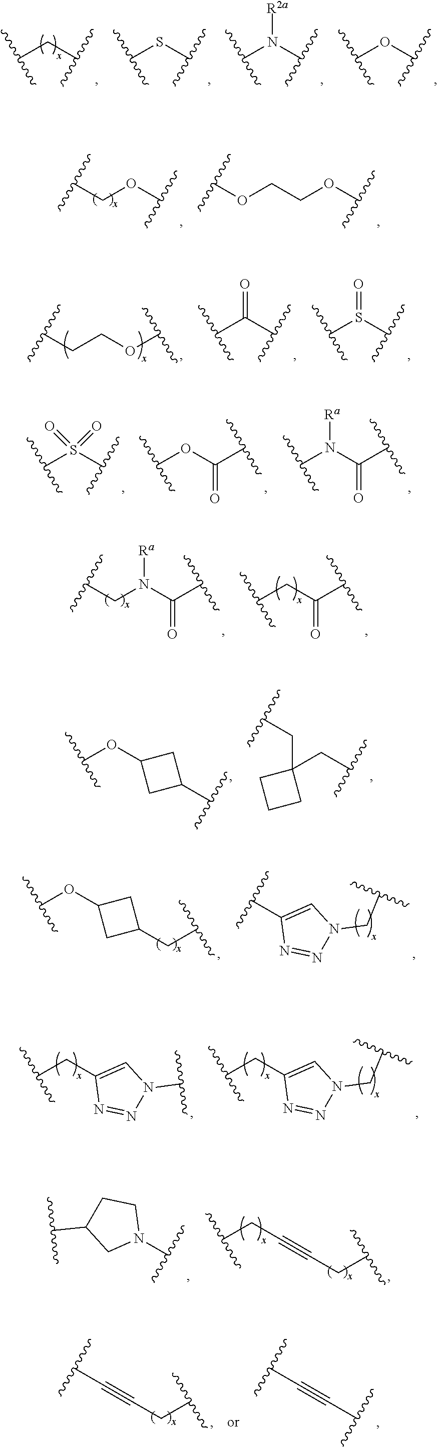
wherein each U is independently unsubstituted or substituted with x R^{2b};
n is 1, 2, 3, 4, 5, or 6;
each x is independently 1, 2, 3, 4, 5, 6, 7, or 8;
each R^{2a} is independently hydrogen or substituted or unsubstituted C₁-C₆alkyl; and
each R^{2b} is independently hydrogen, halogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted 3- to 8-membered heterocycloalkyl, substituted or unsubstituted aryl, substituted or unsubstituted heteroaryl, —CN, —NO₂, —OH, —OR^{a}, —OC(═O)R^{a}, —OC(═O)N(R^{b})₂, —OC(═O)OR^{a}, —SR^{b}, —S(═O)R^{a}, —S(═O)₂R^{a}, —S(═O)₂OR^{b}, —S(═O)₂N(R^{b})₂, —N(R^{b})₂, —NR^{b}C(═O)N(R^{b})₂, —NR^{b}C(═O)R^{a}, —NR^{b}C(═O)OR^{a}, —NR^{b}S(═O)₂R^{a}, —C(═O)R^{b}, —C(═O)OR^{b}, or —C(═O)N(R^{b});
each R^{a} is independently substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
each R^{b} is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted monocyclic 3- to 8-membered heterocycloalkyl, substituted or unsubstituted aryl, or substituted or unsubstituted monocyclic heteroaryl;
or two R^{b} on the same N atom are taken together with the N atom to which they are attached to form a substituted or unsubstituted N-containing heterocycle.

99. The compound of claim 98, or a pharmaceutically acceptable salt or solvate thereof, wherein:
each A is independently absent, 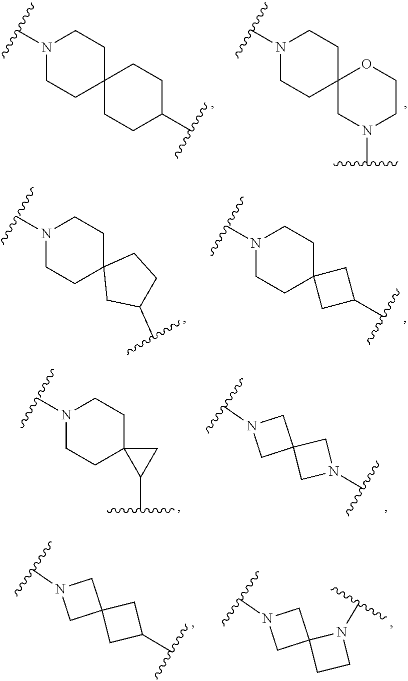 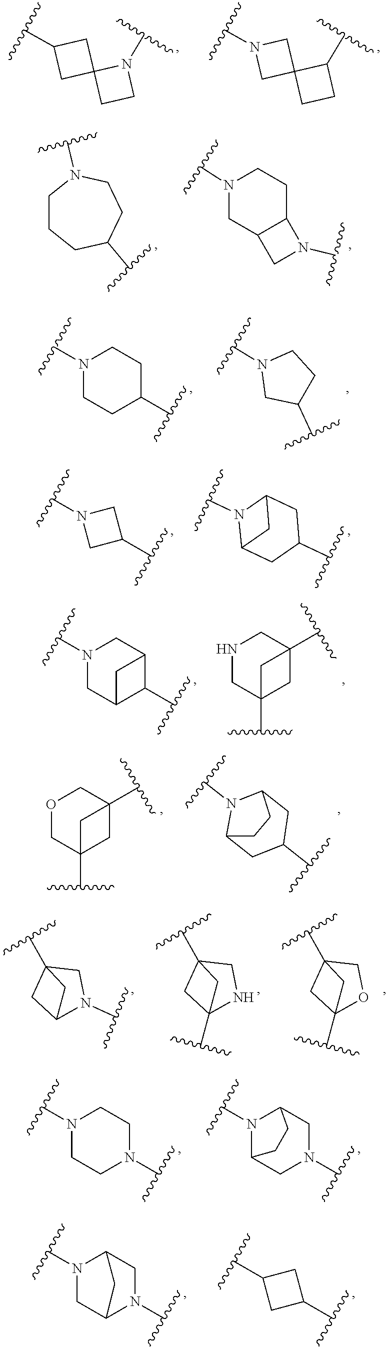 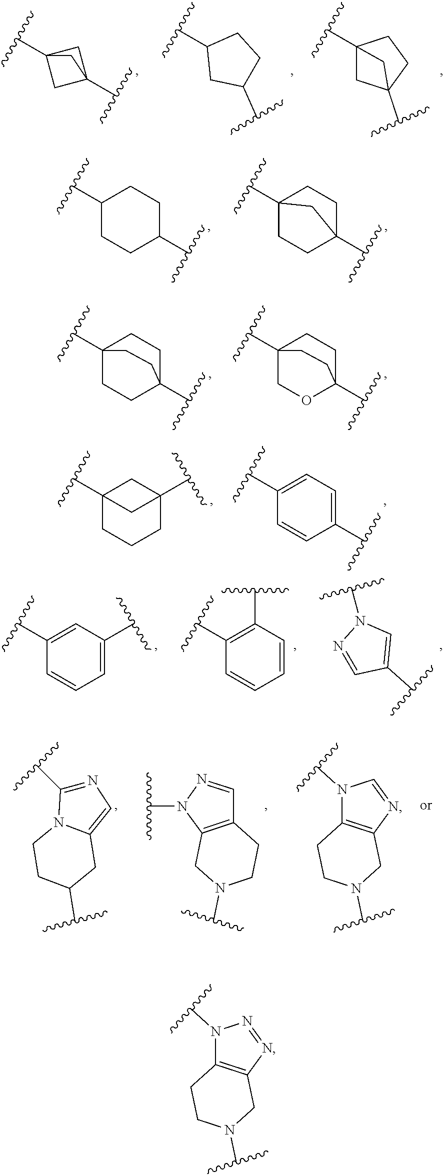
wherein each A is independently unsubstituted or substituted with x R^{2b};
(L¹)ₙ is absent

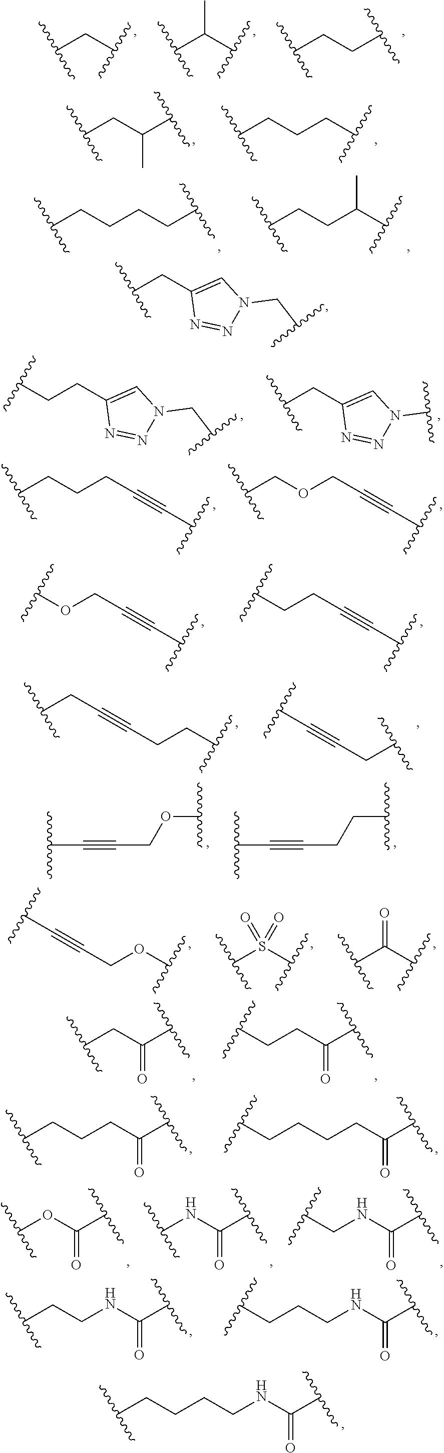

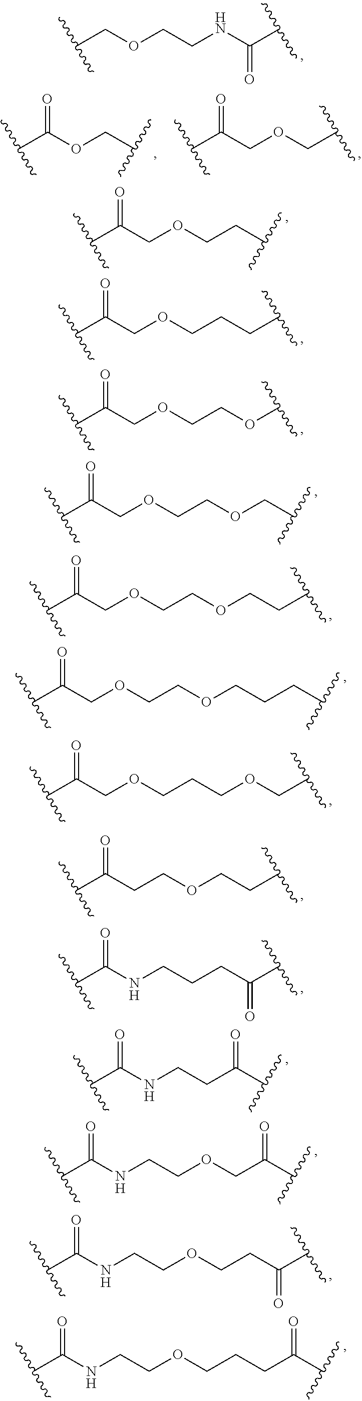

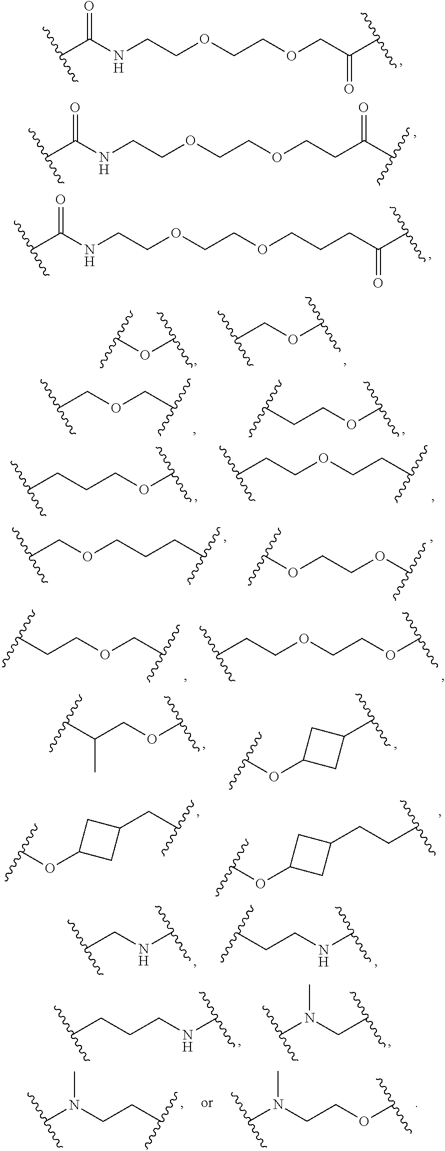

100. The compound of any one of claims 1-95, or a pharmaceutically acceptable salt or solvate thereof, wherein L is absent or 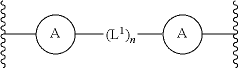
wherein:
each A is independently absent, 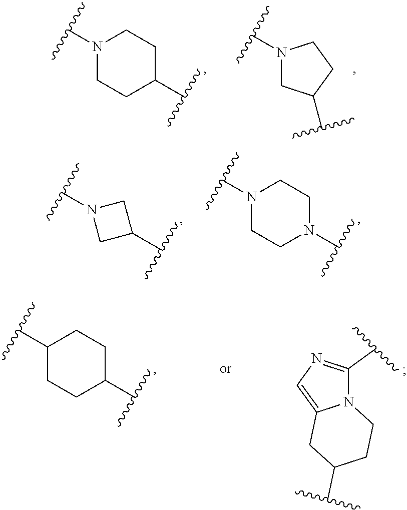 [SMILES: *C1CCN(*)CC1 ; *C1CCN(*)C1 ; *C1CN(*)C1 ; *N1CCN(*)CC1 ; *C1CCC(*)CC1 ; *c1ncc2n1CCC(*)C2]
each L¹ is independently absent, 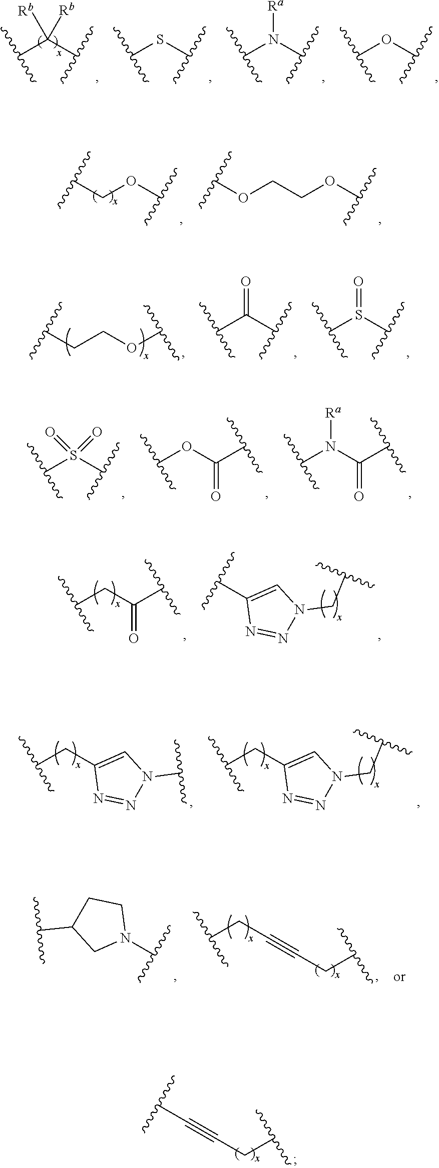
n is 1, 2, or 3; each x is independently 1, 2, 3, 4, 5, or 6;
each R^{a} is independently hydrogen or substituted or unsubstituted C₁-C₆alkyl; and
each R^{b} is independently hydrogen, halogen, or substituted or unsubstituted C₁-C₆alkyl.

101. The compound of any one of claims 98-100, or a pharmaceutically acceptable salt or solvate thereof, wherein:
(L¹)ₙ is absent,

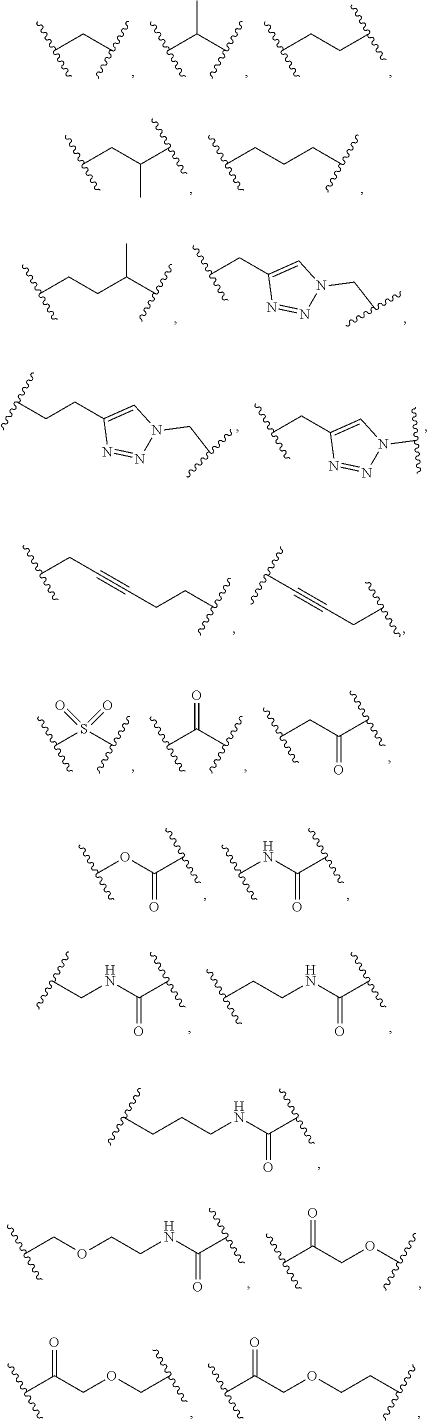

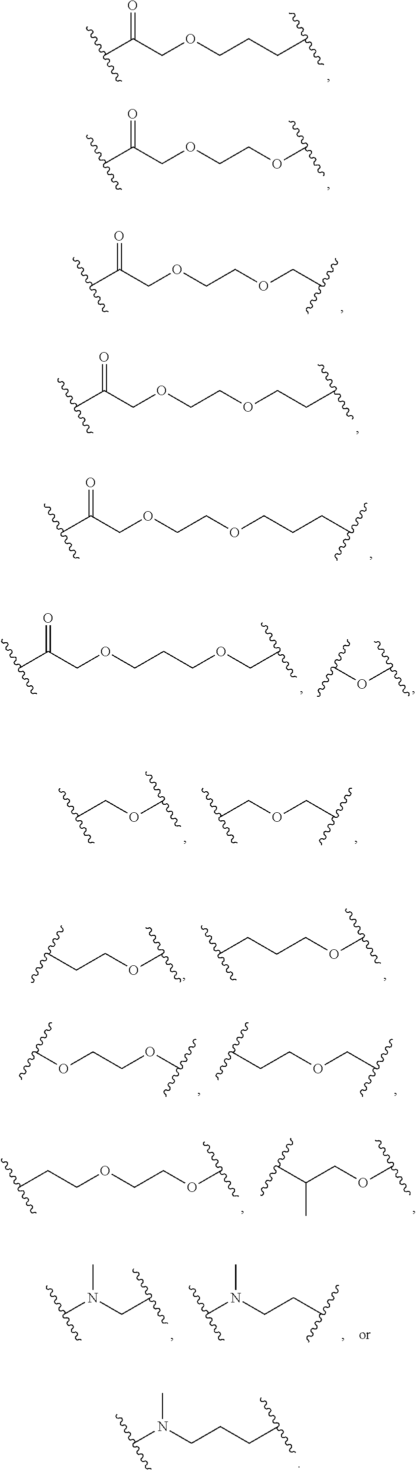

102. The compound of any one of claims 1-95, or a pharmaceutically acceptable salt or solvate thereof, wherein L has one of the following structures: 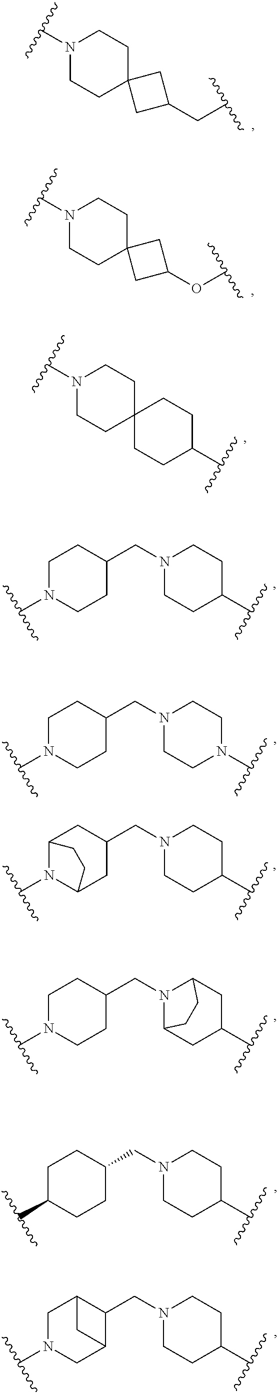 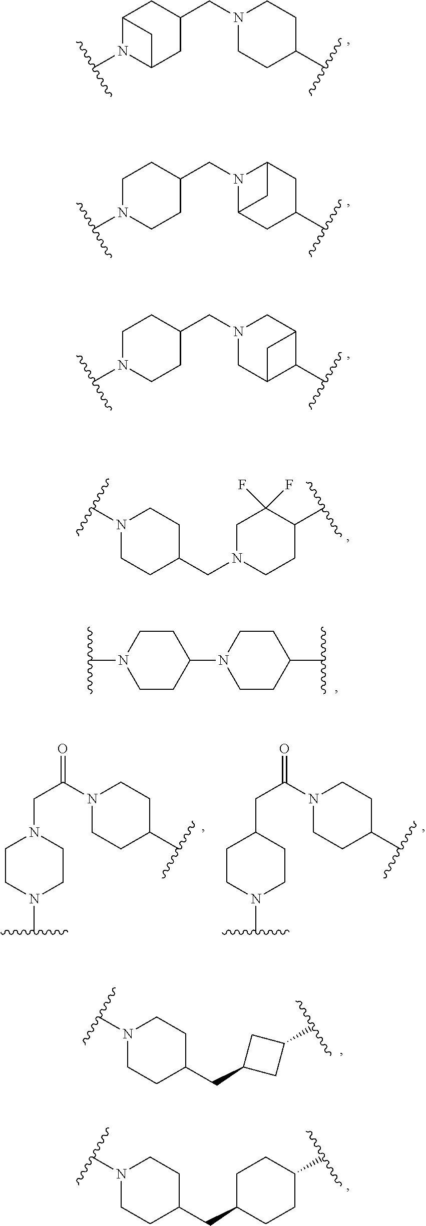 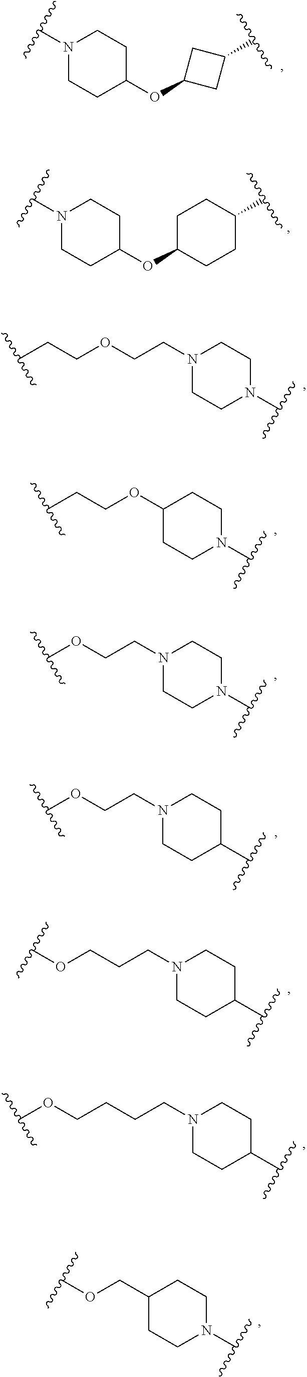 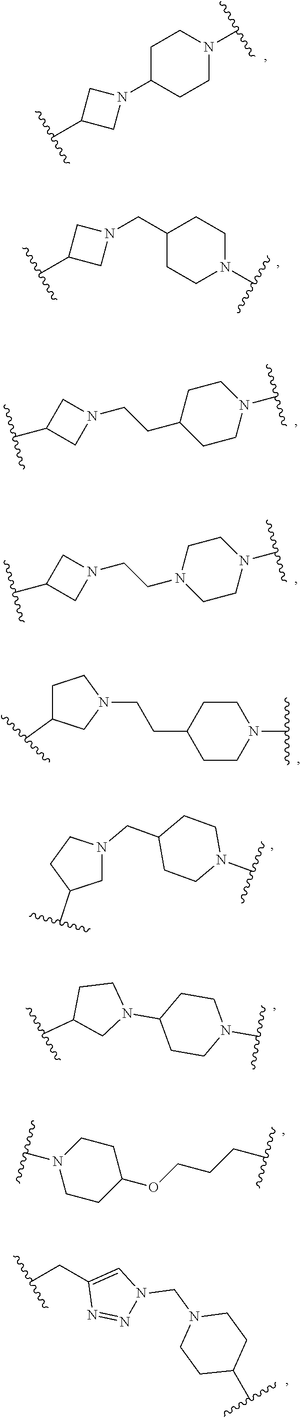 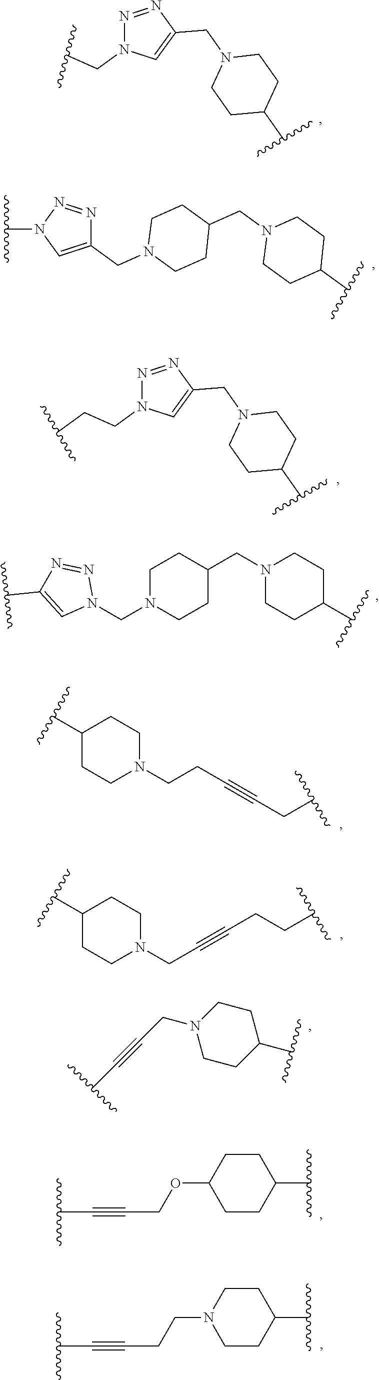 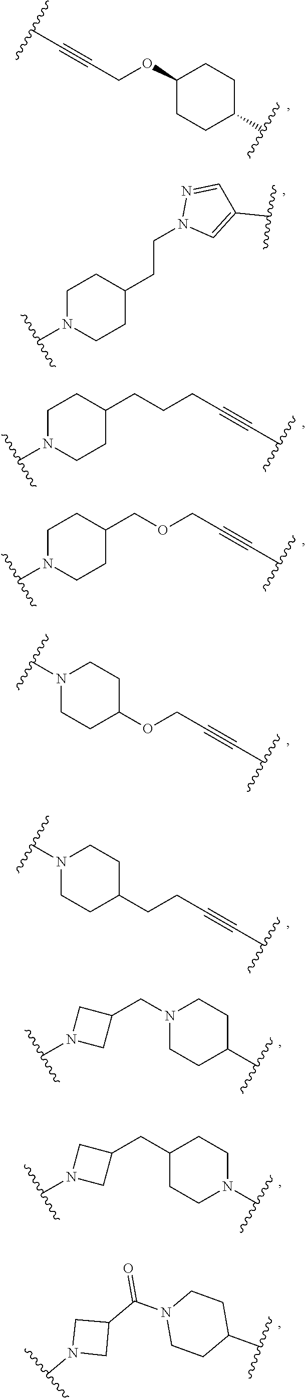 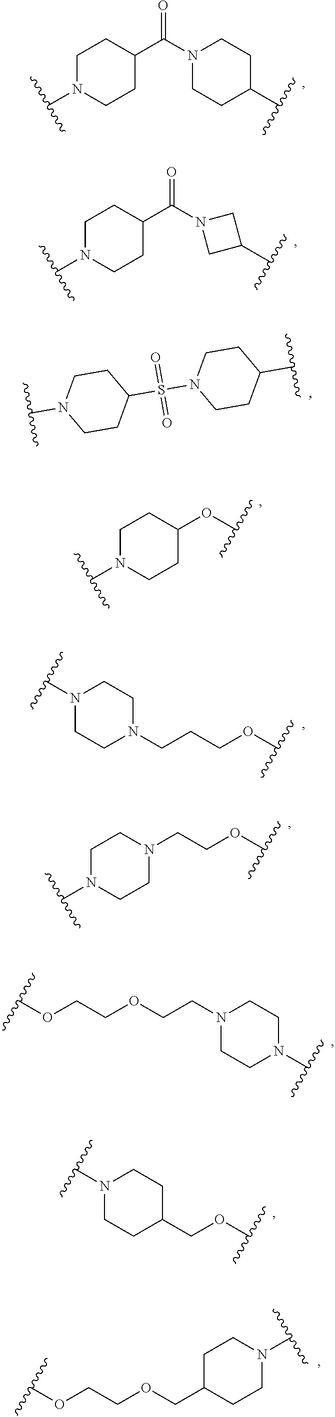 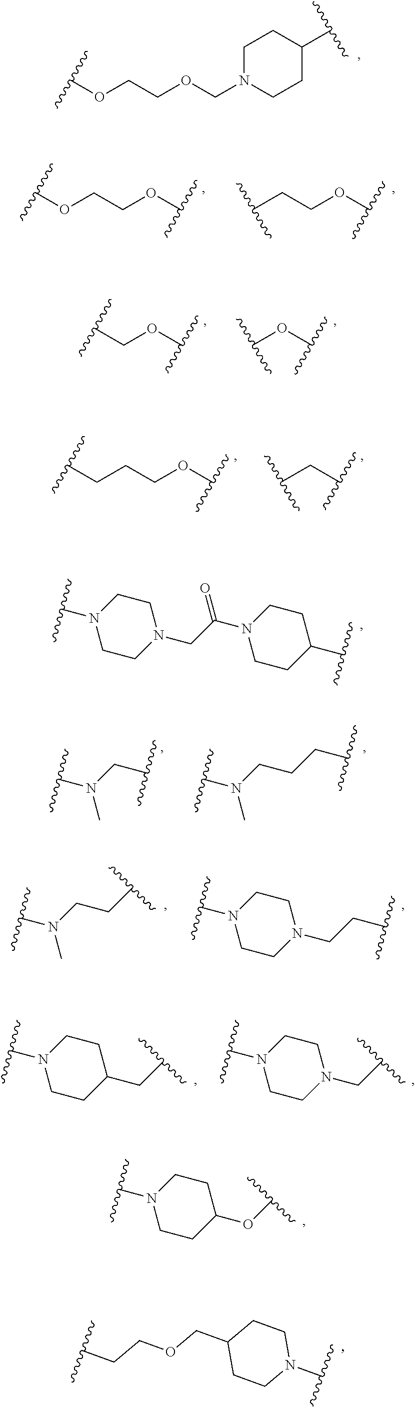 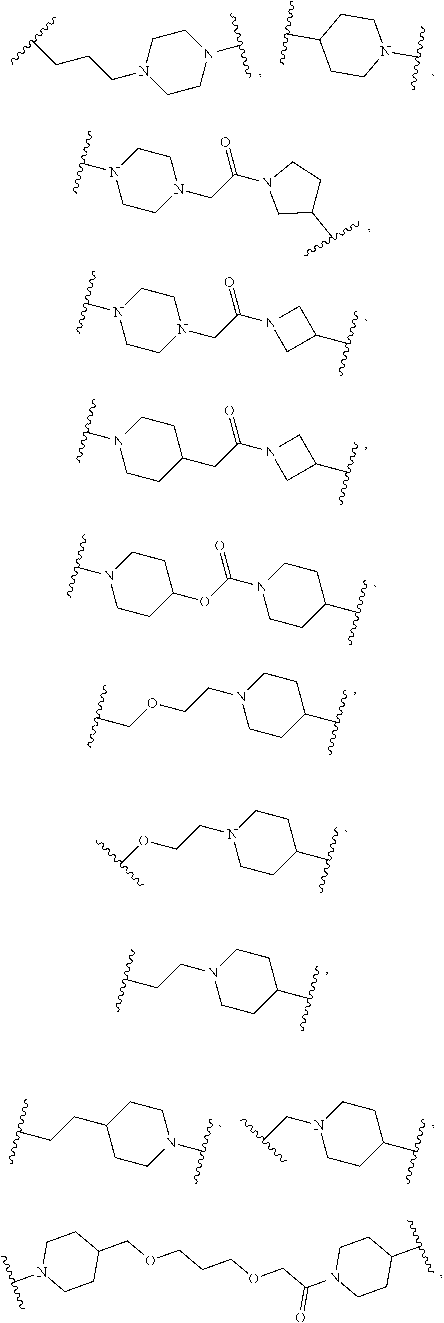 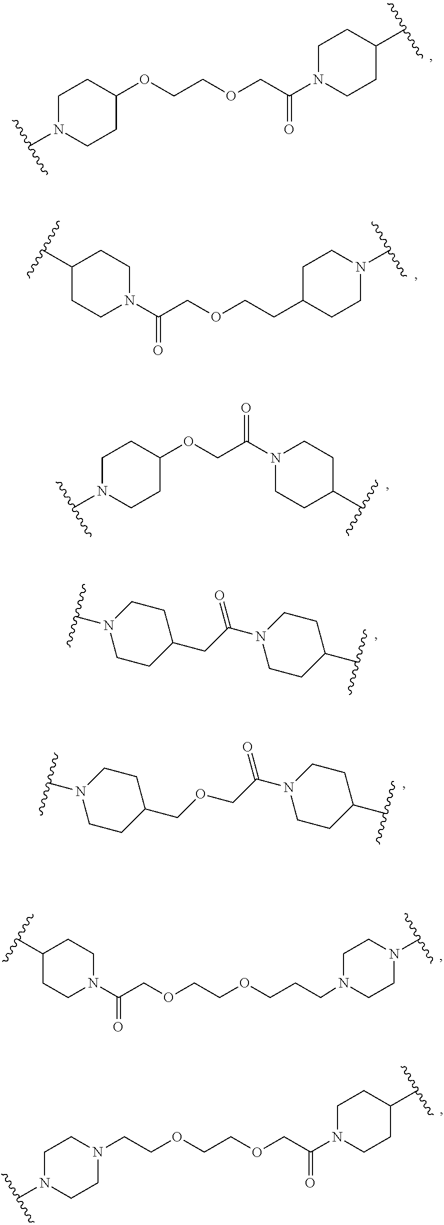 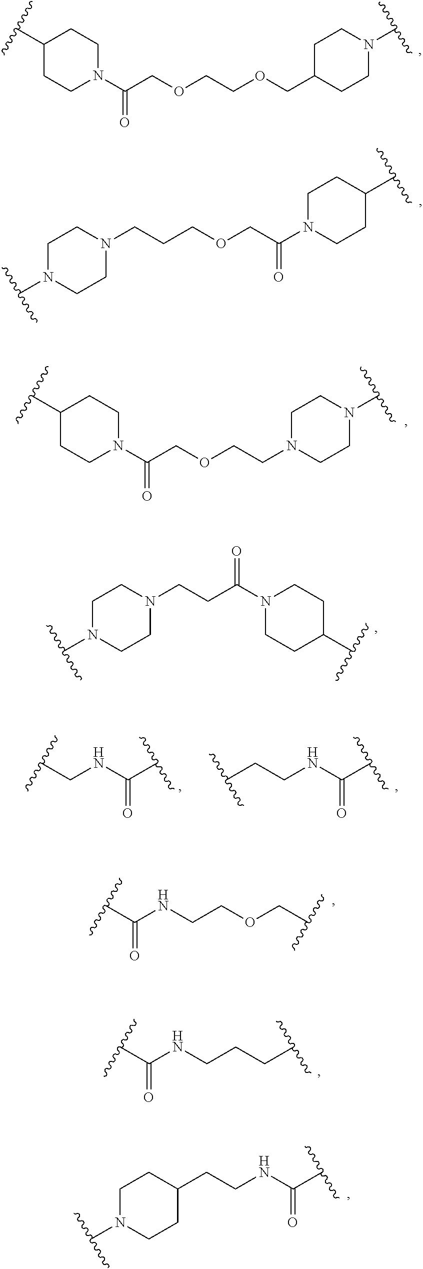 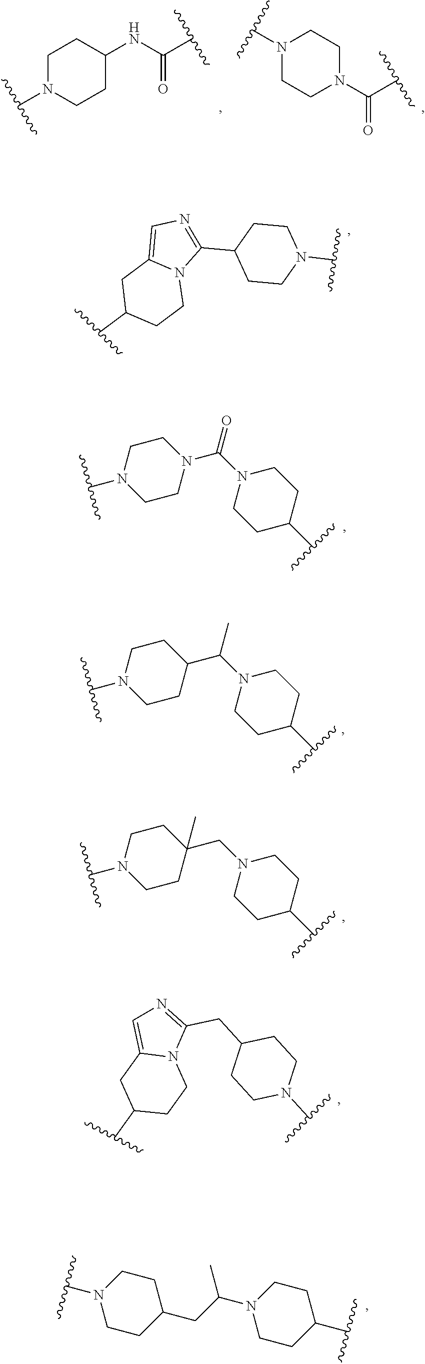 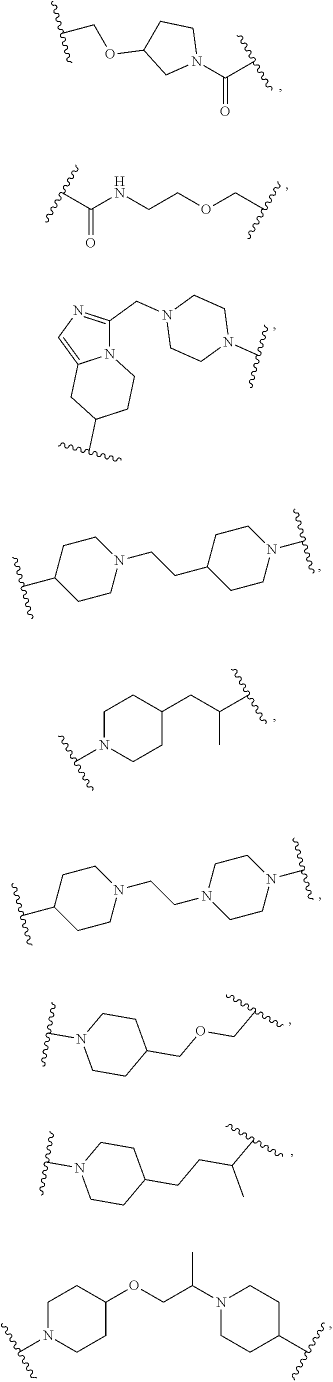 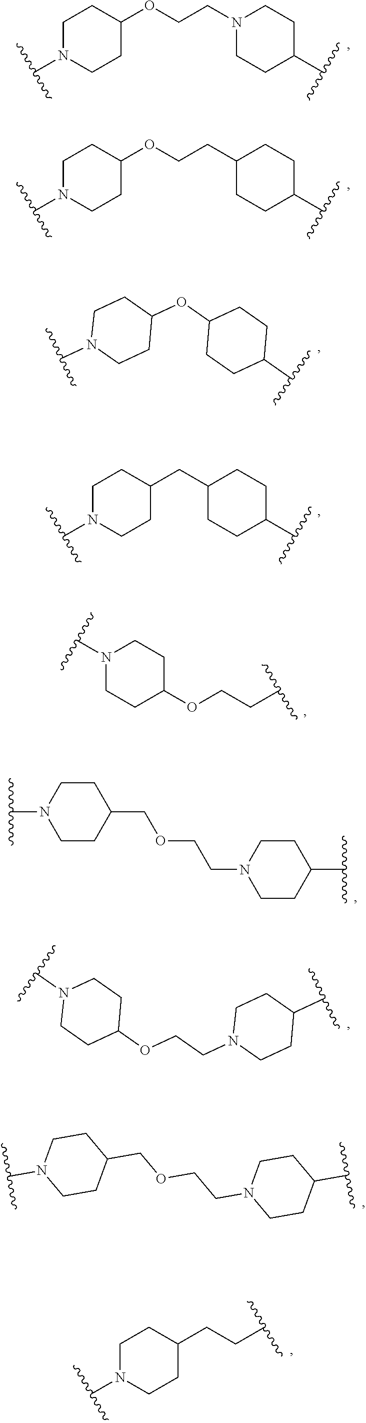 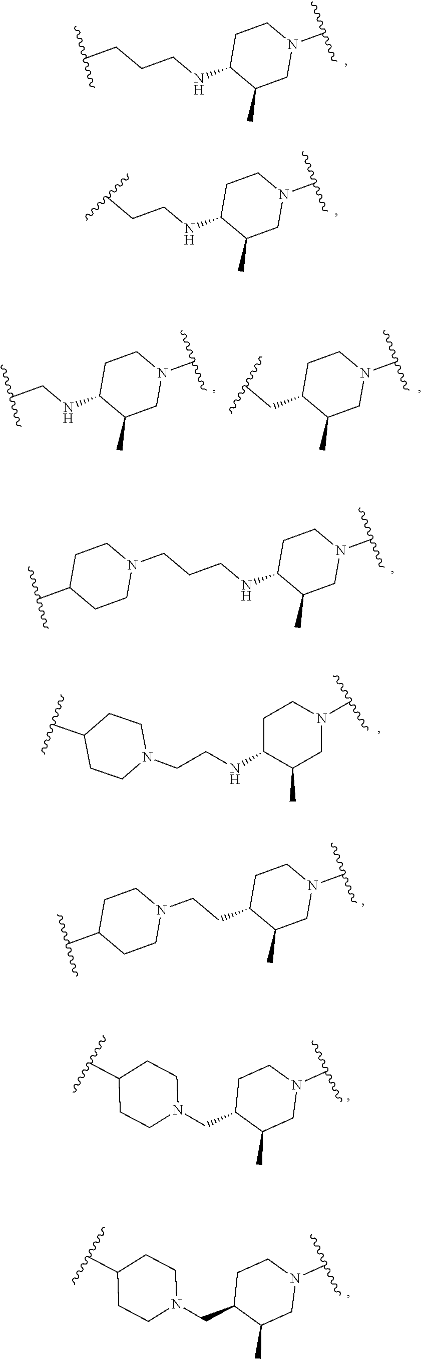 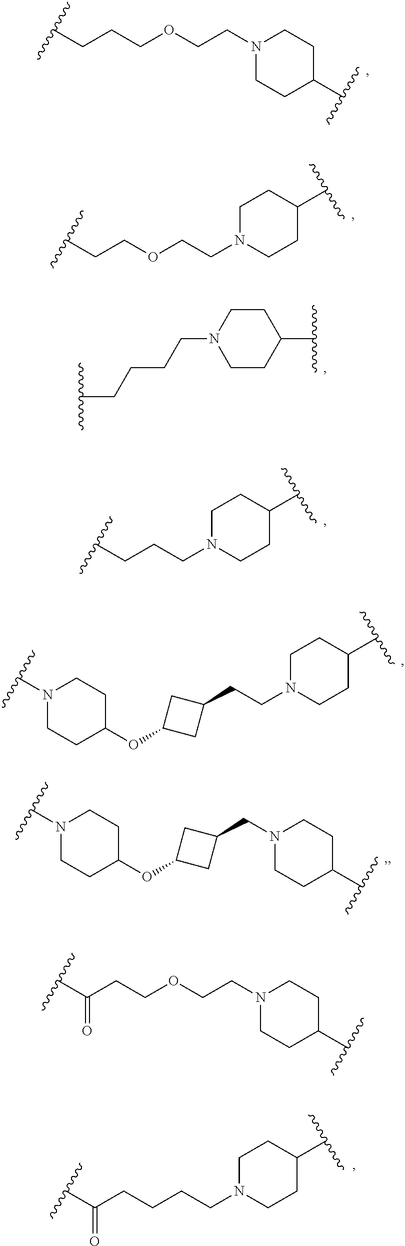 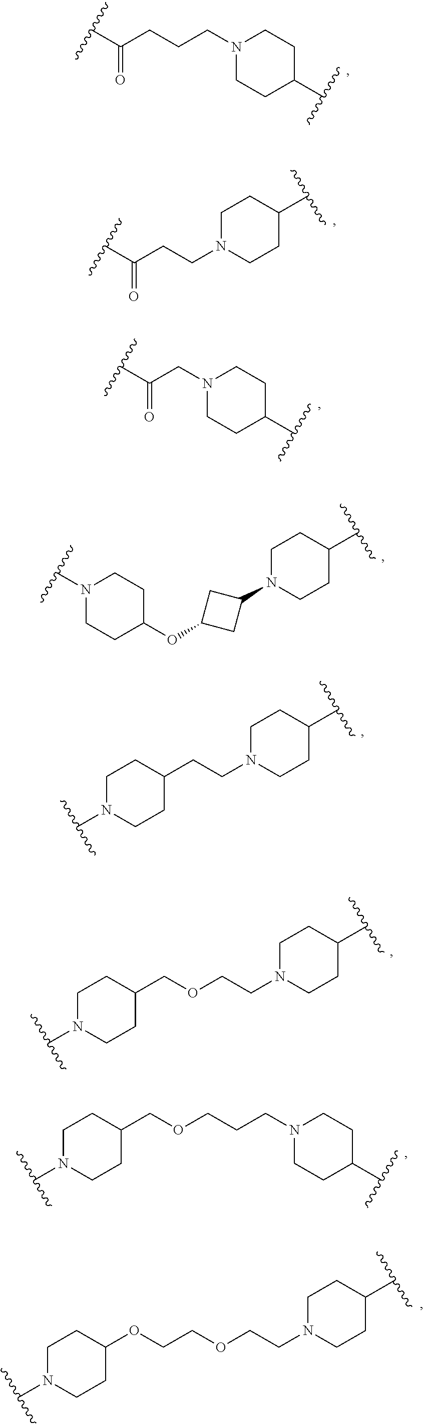 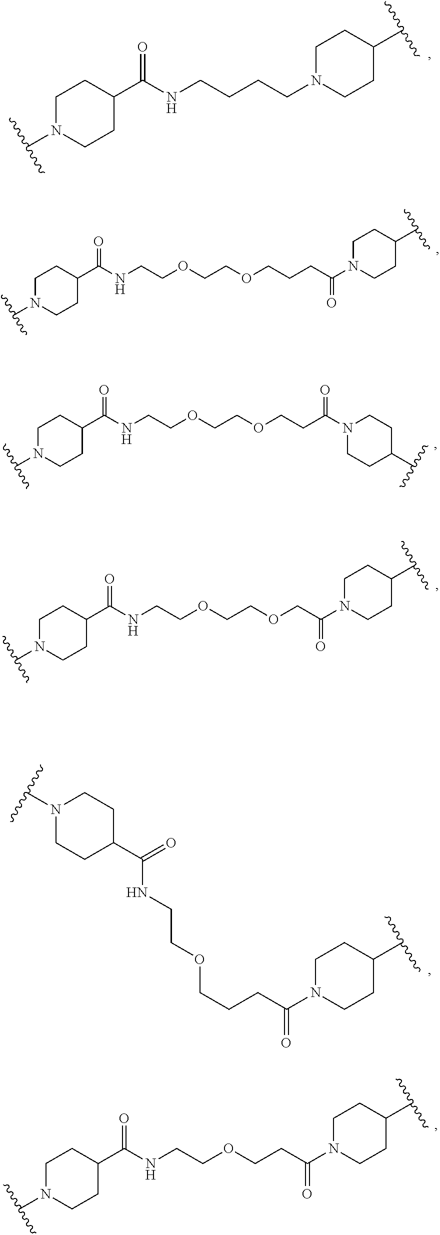 [SMILES: *C1CCN(CCCCNC(=O)C2CCN(*)CC2)CC1 ; *C1CCN(C(=O)CCCOCCOCCNC(=O)C2CCN(*)CC2)CC1 ; *C1CCN(C(=O)CCOCCOCCNC(=O)C2CCN(*)CC2)CC1 ; *C1CCN(C(=O)COCCOCCNC(=O)C2CCN(*)CC2)CC1 ; *C1CCN(C(=O)CCCOCCNC(=O)C2CCN(*)CC2)CC1 ; *C1CCN(C(=O)CCOCCNC(=O)C2CCN(*)CC2)CC1] 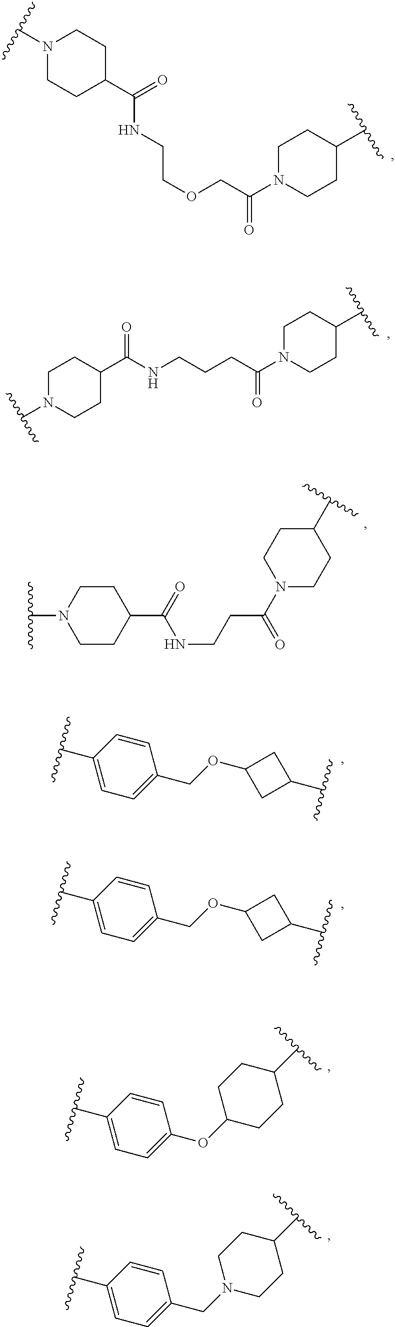 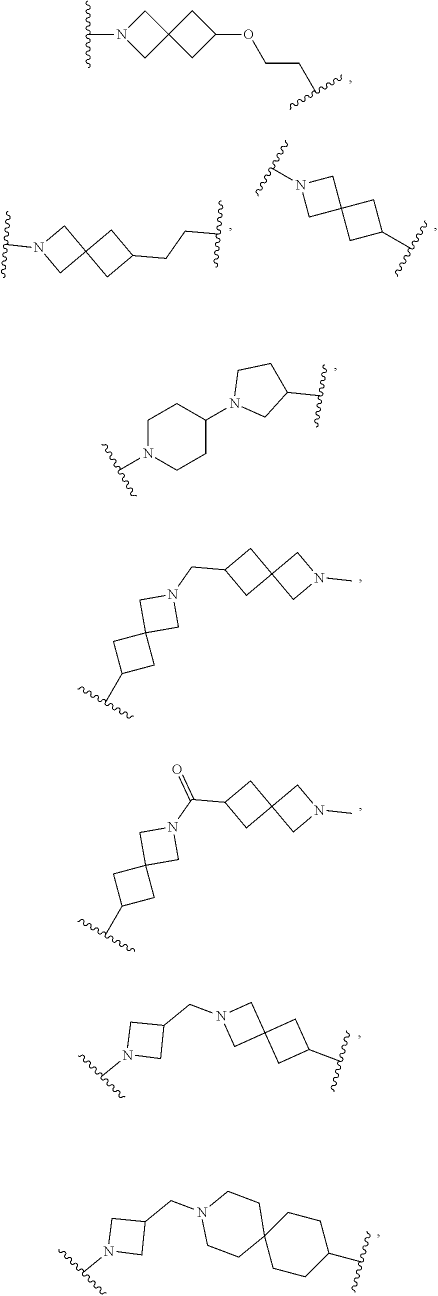 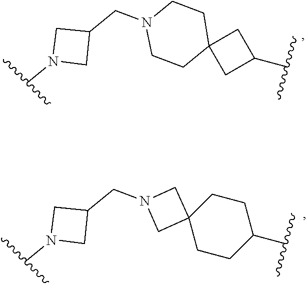 [SMILES: *C1CC2(CCN(CC3CN(*)C3)CC2)C1 ; *C1CCC2(CC1)CN(CC1CN(*)C1)C2] 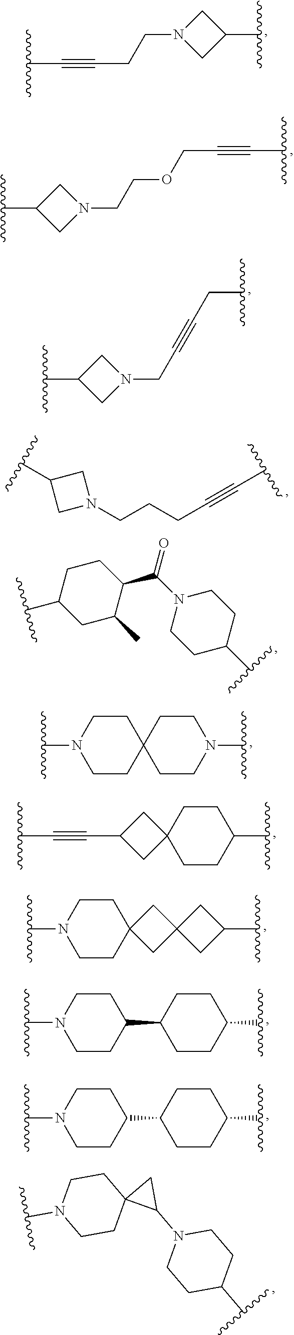 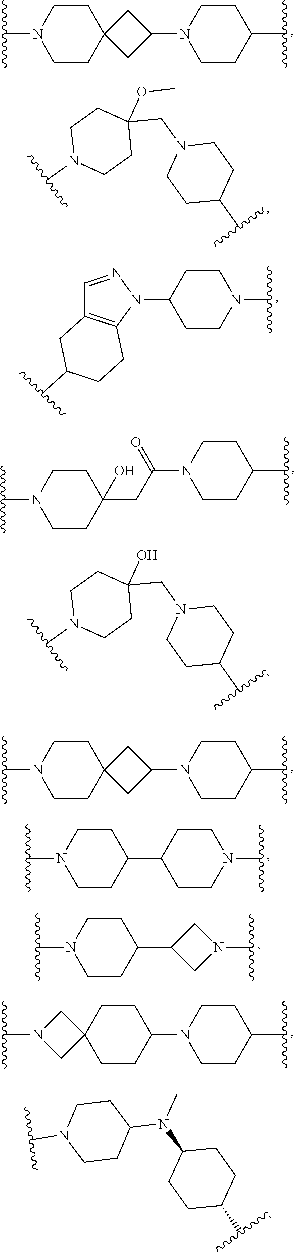 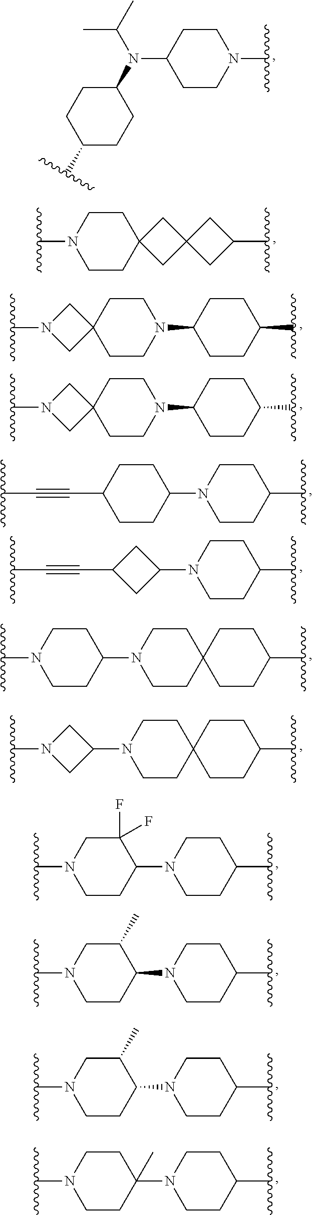 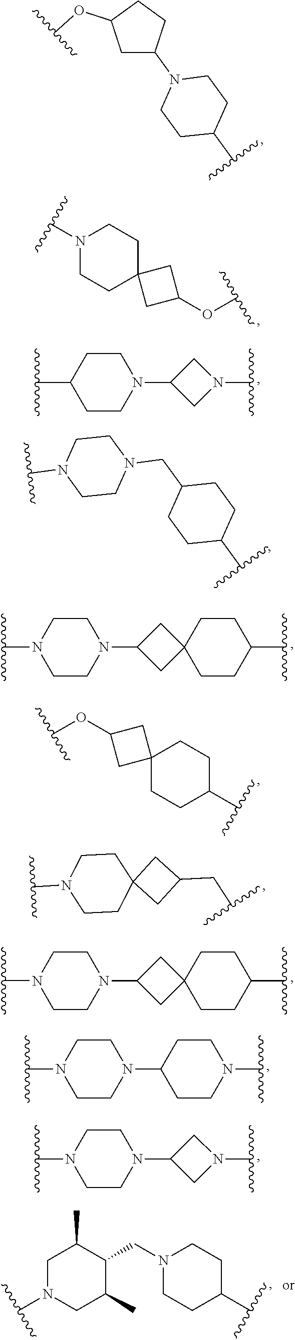 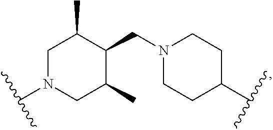 [SMILES: *C1CCN(C[C@@H]2[C@H](C)CN(*)C[C@@H]2C)CC1]
or a pharmaceutically acceptable salt or solvate thereof.

103. The compound of any one of claims 1-95, or a pharmaceutically acceptable salt or solvate thereof, wherein L has one of the following structures: 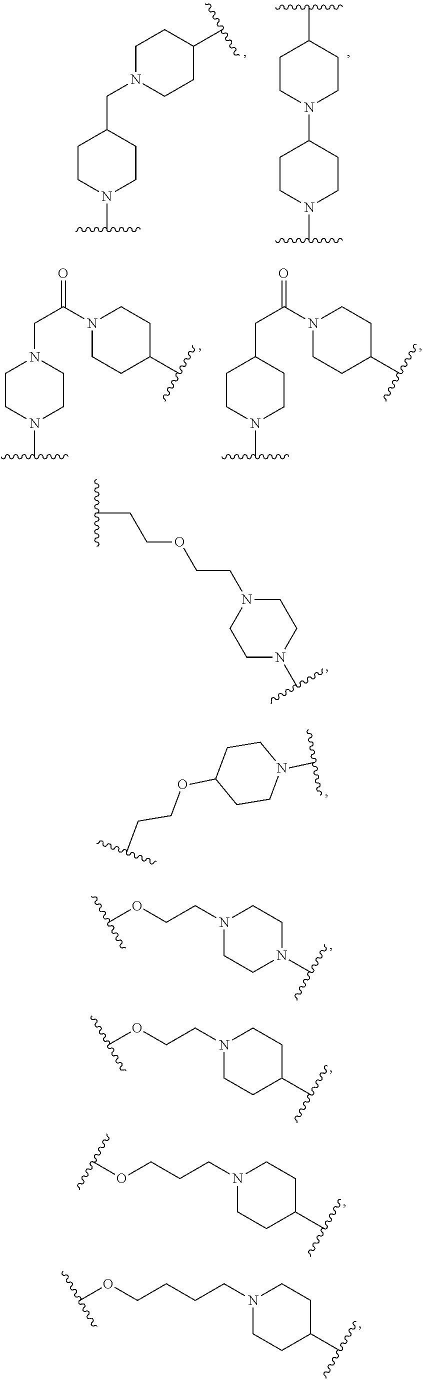 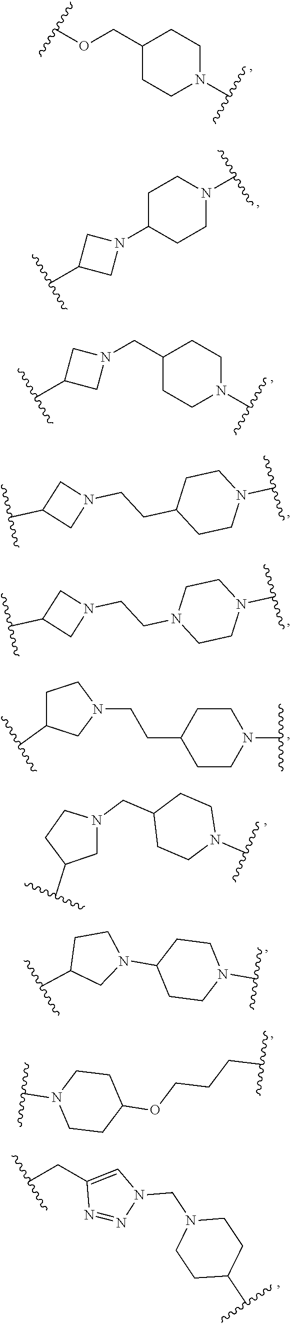 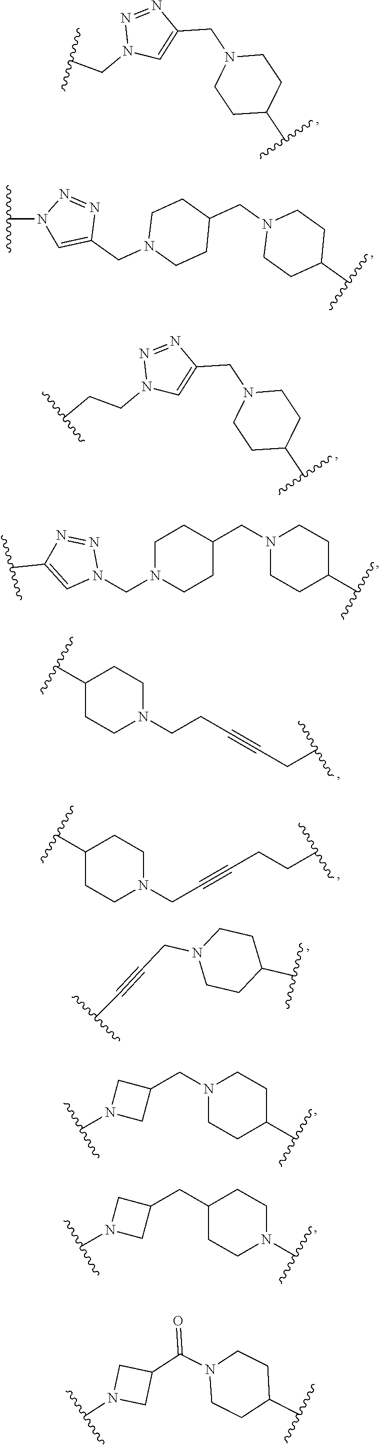 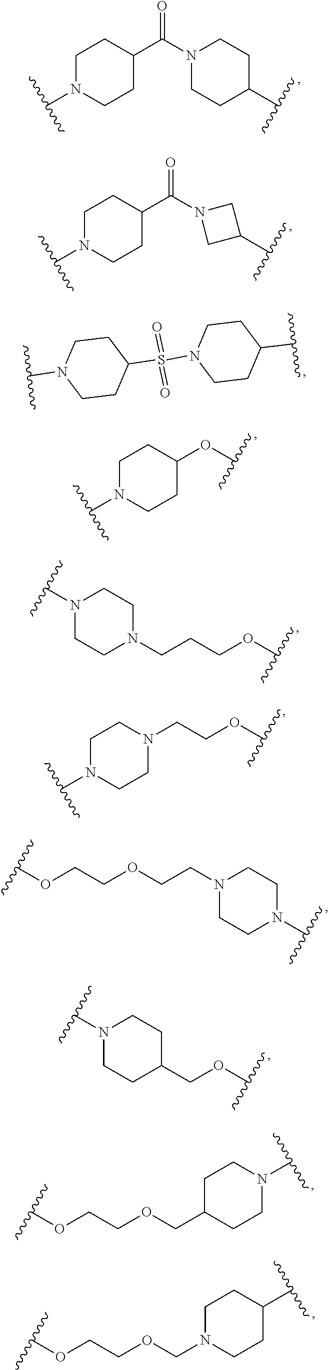 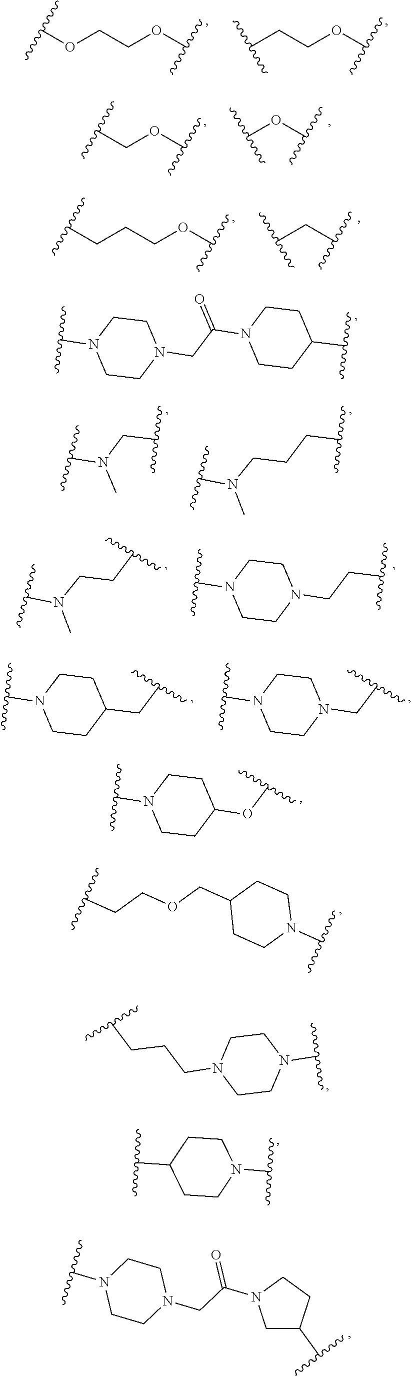 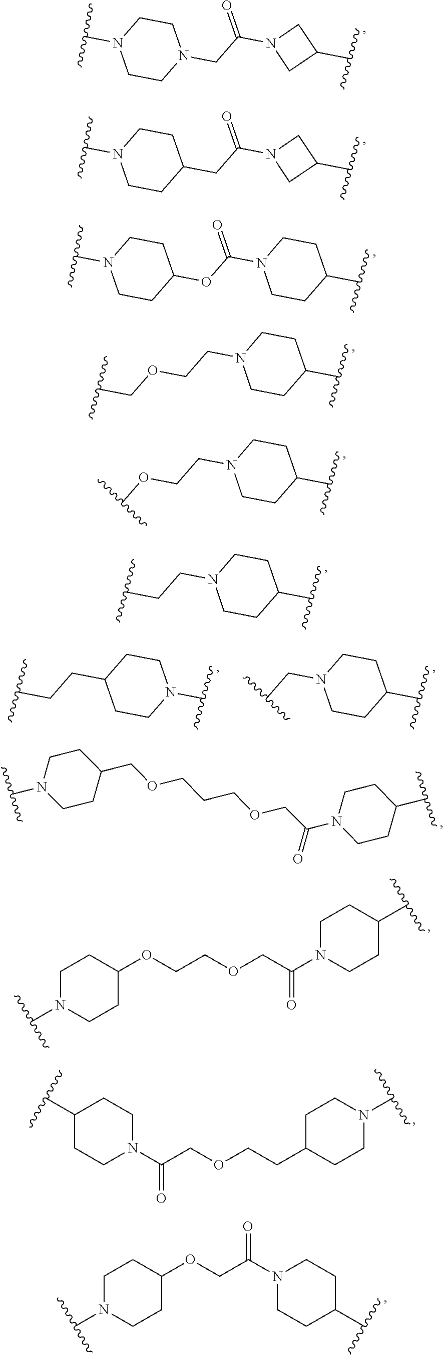 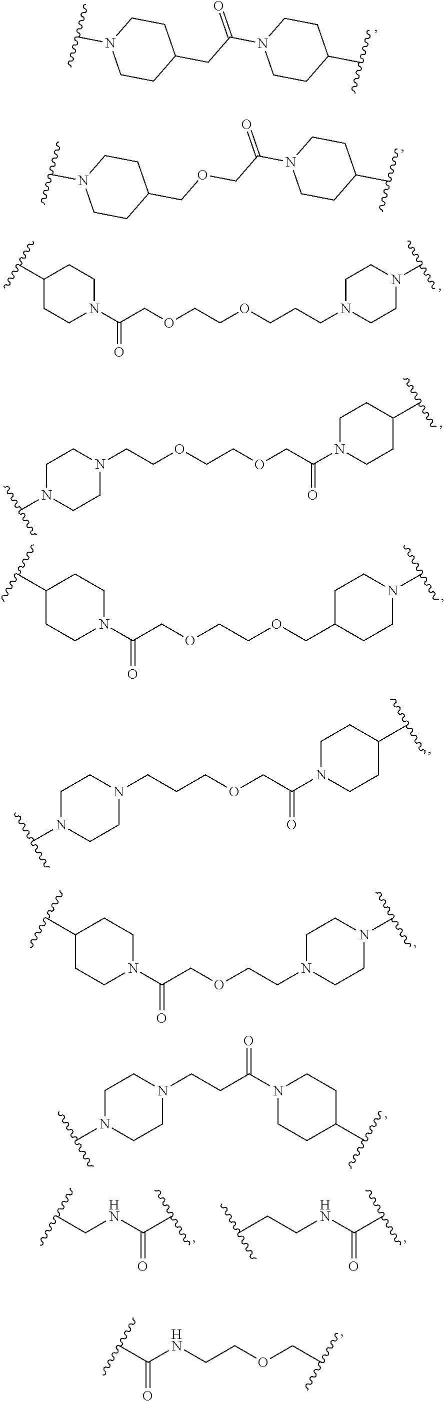 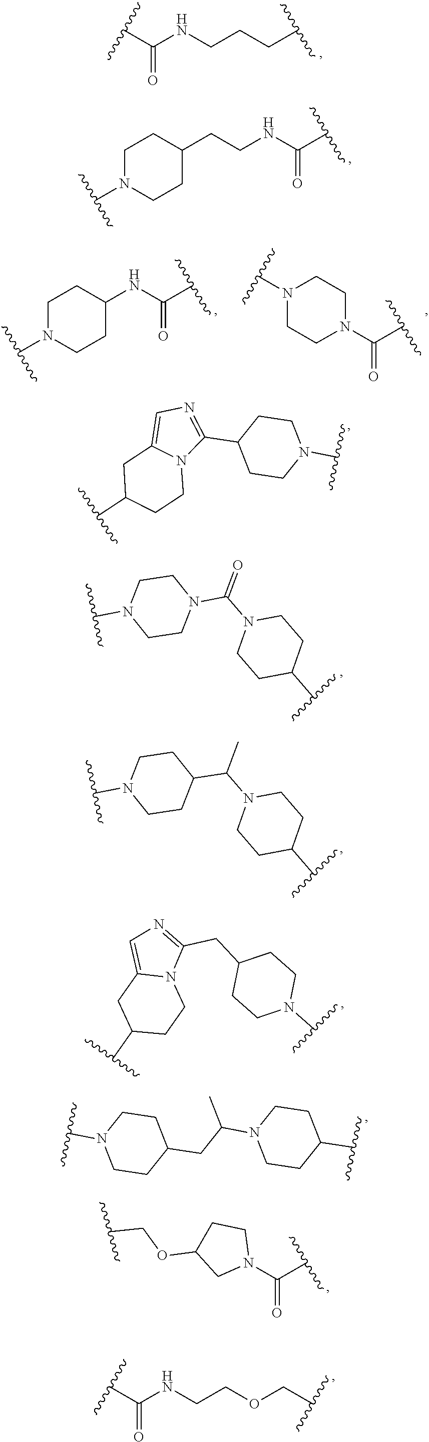 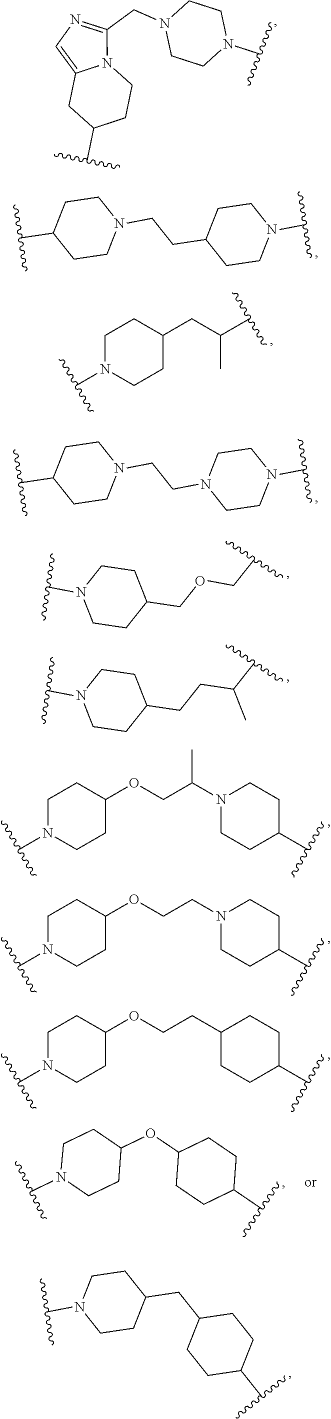
or a pharmaceutically acceptable salt or solvate thereof.

104. A compound, or a pharmaceutically acceptable salt or solvate thereof, that has the following structure: 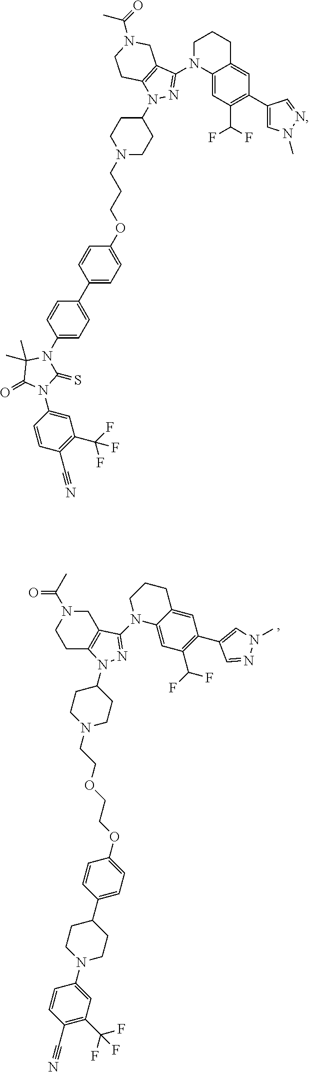 [SMILES: CC(=O)N1CCc2c(c(N3CCCc4cc(-c5cnn(C)c5)c(C(F)F)cc43)nn2C2CCN(CCCOc3ccc(-c4ccc(N5C(=S)N(c6ccc(C#N)c(C(F)(F)F)c6)C(=O)C5(C)C)cc4)cc3)CC2)C1 ; CC(=O)N1CCc2c(c(N3CCCc4cc(-c5cnn(C)c5)c(C(F)F)cc43)nn2C2CCN(CCOCCOc3ccc(C4CCN(c5ccc(C#N)c(C(F)(F)F)c5)CC4)cc3)CC2)C1 ; ","] 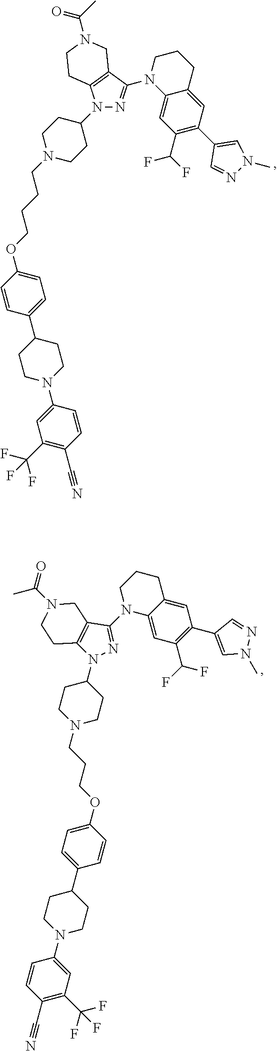 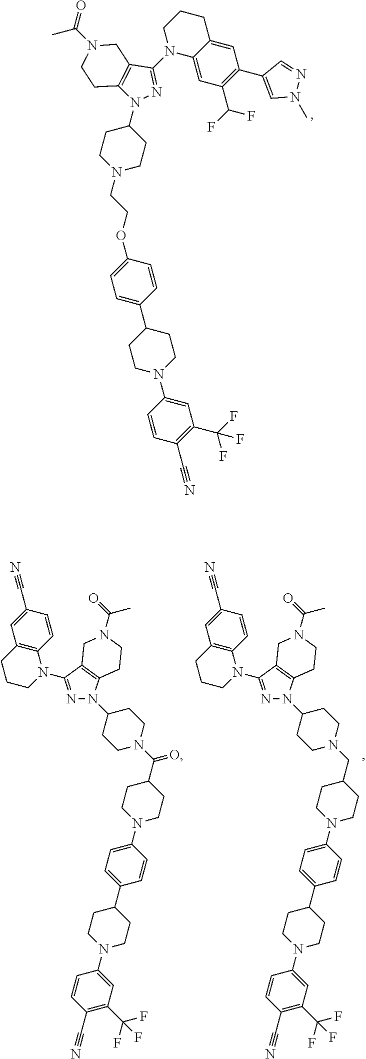 [SMILES: CC(=O)N1CCc2c(c(N3CCCc4cc(-c5cnn(C)c5)c(C(F)F)cc43)nn2C2CCN(CCOc3ccc(C4CCN(c5ccc(C#N)c(C(F)(F)F)c5)CC4)cc3)CC2)C1 ; "," ; CC(=O)N1CCc2c(c(N3CCCc4cc(C#N)ccc43)nn2C2CCN(C(=O)C3CCN(c4ccc(C5CCN(c6ccc(C#N)c(C(F)(F)F)c6)CC5)cc4)CC3)CC2)C1 ; CC(=O)N1CCc2c(c(N3CCCc4cc(C#N)ccc43)nn2C2CCN(CC3CCN(c4ccc(C5CCN(c6ccc(C#N)c(C(F)(F)F)c6)CC5)cc4)CC3)CC2)C1 ; ","] 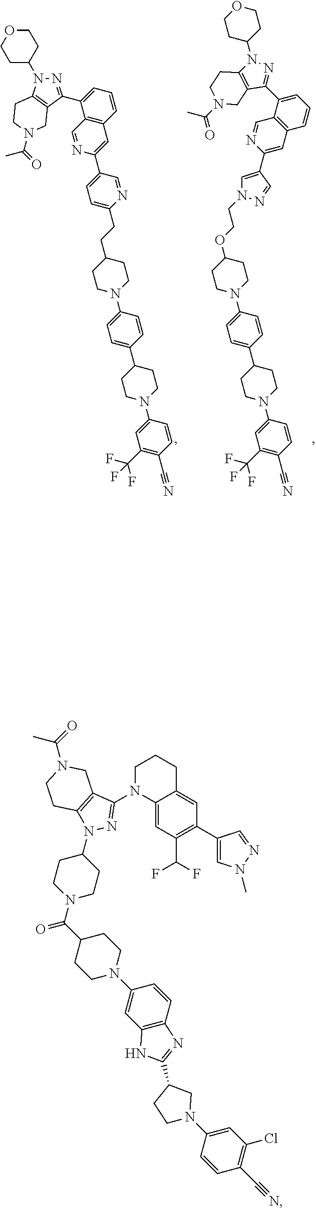 [SMILES: CC(=O)N1CCc2c(c(-c3cccc4cc(-c5ccc(CCC6CCN(c7ccc(C8CCN(c9ccc(C#N)c(C(F)(F)F)c9)CC8)cc7)CC6)nc5)ncc34)nn2C2CCOCC2)C1 ; "," ; CC(=O)N1CCc2c(c(-c3cccc4cc(-c5cnn(CCOC6CCN(c7ccc(C8CCN(c9ccc(C#N)c(C(F)(F)F)c9)CC8)cc7)CC6)c5)ncc34)nn2C2CCOCC2)C1 ; "," ; CC(=O)N1CCc2c(c(N3CCCc4cc(-c5cnn(C)c5)c(C(F)F)cc43)nn2C2CCN(C(=O)C3CCN(c4ccc5nc([C@H]6CCN(c7ccc(C#N)c(Cl)c7)C6)[nH]c5c4)CC3)CC2)C1] 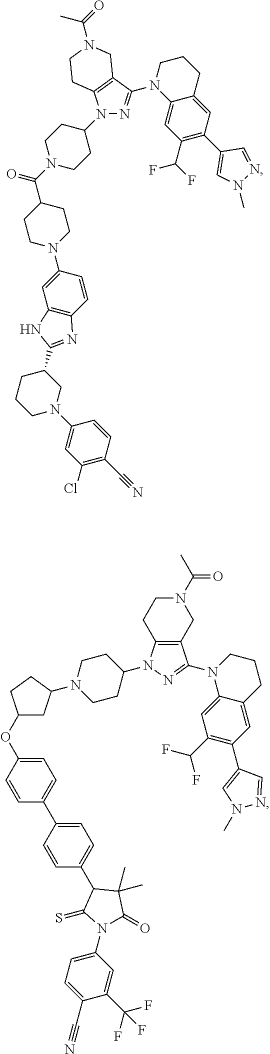 [SMILES: CC(=O)N1CCc2c(c(N3CCCc4cc(-c5cnn(C)c5)c(C(F)F)cc43)nn2C2CCN(C(=O)C3CCN(c4ccc5nc([C@H]6CCCN(c7ccc(C#N)c(Cl)c7)C6)[nH]c5c4)CC3)CC2)C1 ; CC(=O)N1CCc2c(c(N3CCCc4cc(-c5cnn(C)c5)c(C(F)F)cc43)nn2C2CCN(C3CCC(Oc4ccc(-c5ccc(C6C(=S)N(c7ccc(C#N)c(C(F)(F)F)c7)C(=O)C6(C)C)cc5)cc4)C3)CC2)C1] 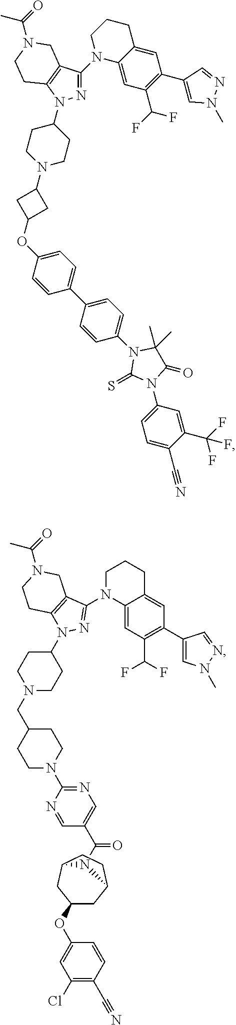 [SMILES: CC(=O)N1CCc2c(c(N3CCCc4cc(-c5cnn(C)c5)c(C(F)F)cc43)nn2C2CCN(C3CC(Oc4ccc(-c5ccc(N6C(=S)N(c7ccc(C#N)c(C(F)(F)F)c7)C(=O)C6(C)C)cc5)cc4)C3)CC2)C1 ; CC(=O)N1CCc2c(c(N3CCCc4cc(-c5cnn(C)c5)c(C(F)F)cc43)nn2C2CCN(CC3CCN(c4ncc(C(=O)N5[C@@H]6CC[C@H]5C[C@@H](Oc5ccc(C#N)c(Cl)c5)C6)cn4)CC3)CC2)C1] 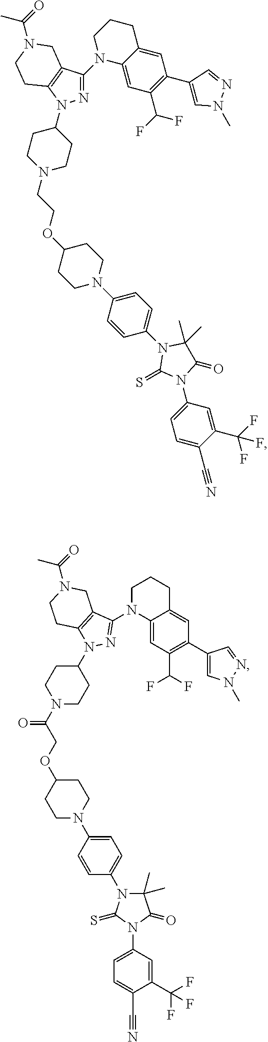 [SMILES: CC(=O)N1CCc2c(c(N3CCCc4cc(-c5cnn(C)c5)c(C(F)F)cc43)nn2C2CCN(CCOC3CCN(c4ccc(N5C(=S)N(c6ccc(C#N)c(C(F)(F)F)c6)C(=O)C5(C)C)cc4)CC3)CC2)C1 ; CC(=O)N1CCc2c(c(N3CCCc4cc(-c5cnn(C)c5)c(C(F)F)cc43)nn2C2CCN(C(=O)COC3CCN(c4ccc(N5C(=S)N(c6ccc(C#N)c(C(F)(F)F)c6)C(=O)C5(C)C)cc4)CC3)CC2)C1] 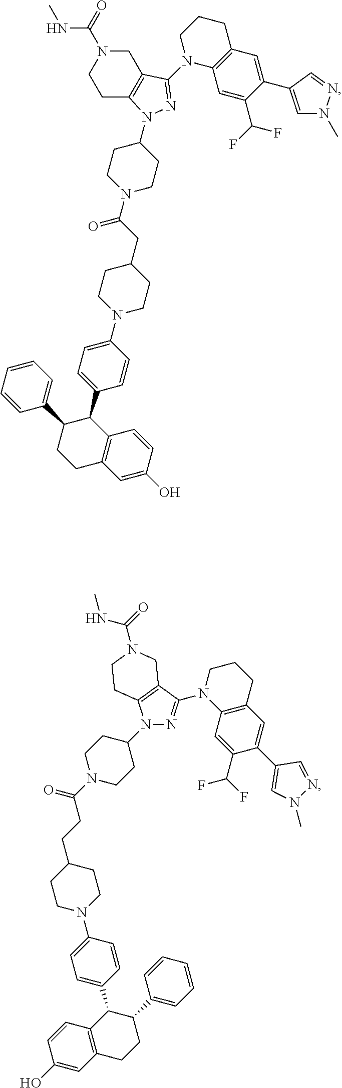 [SMILES: CNC(=O)N1CCc2c(c(N3CCCc4cc(-c5cnn(C)c5)c(C(F)F)cc43)nn2C2CCN(C(=O)CC3CCN(c4ccc([C@H]5c6ccc(O)cc6CC[C@H]5c5ccccc5)cc4)CC3)CC2)C1 ; CNC(=O)N1CCc2c(c(N3CCCc4cc(-c5cnn(C)c5)c(C(F)F)cc43)nn2C2CCN(C(=O)CCC3CCN(c4ccc([C@H]5c6ccc(O)cc6CC[C@H]5c5ccccc5)cc4)CC3)CC2)C1] 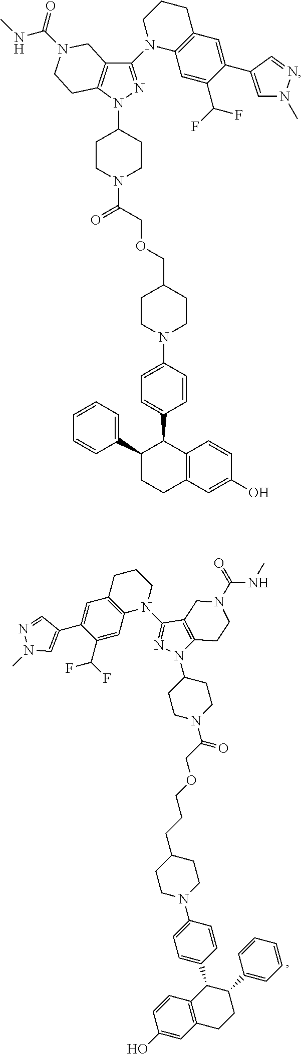 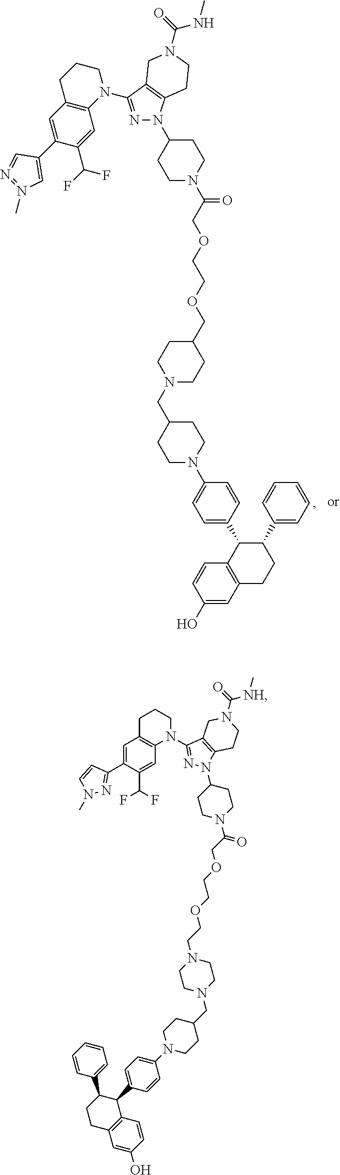
or a pharmaceutically acceptable salt or solvate thereof.

105. A stable ternary complex comprising:
a. one or more disease-dependent proteins (DDPs);
b. a disease protein (DP); and
c. heterobifunctional conditional inhibitor compound of Formula (I): 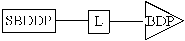
wherein:
SBDDP is a silent binder of a disease-dependent protein (DDP);
L is an optional linker; and
BDP is a binder of a disease protein (DP);
wherein DDP and DP are present in a cell of interest (COI) and the relative abundance of the DP in the COI is greater than the relative abundance of the DDP in the COI.

106. The stable ternary complex of claim 105, wherein DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3.

107. The stable ternary complex of claim 106, wherein DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

108. The compound of claim 106, wherein DDP is a human bromodomain-containing protein (BRD).

109. The compound of claim 106, wherein DDP is a human bromodomain-containing protein that is a Group Ia BRD, Group Ib BRD, Group II BRD, Group IIIa BRD, Group IIIb BRD, Group IIII BRD, Group IV BRD, Group VI BRD, Group VI BRD, Group VIII BRD, or group IX BRD; wherein:
the Group Ia BRD is selected from PCAF, GCN5L2, p300/CBP, TAF1 and TAF1L;
the Group Ib BRD is selected from BRPFTA/B (BR140), BRPF2 (BRD1) BRPF3 and BRD8 (SMAP);
the Group II BRD is selected from histone methyltransferases ASH1L and MLL;
the Group IIIa BRD is selected from chromatin remodeling factors SMARCA2 (BRM), SMARCA4 (BRGT), BRD7, BRD9 and PBRM1 (polybromo);
the Group IIIb BRD is selected from ISWI family chromatin remodeling factors BAZ1A (ACF1), BAZ1B (WSTF, William-Beuren syndrome transcription factor), BAZ2A, BAZ2B, BPTF and CECR2;
the Group IV BRD is selected from ATPase family AAA domain-containing proteins ATAD2 and ATAD2B;
the Group VI BRD is selected from Tripartite-motif-containing (TRIM) family proteins TRIM24 (TIF1α), TRIM28 (TIF1β, KAP1), TRIM33 (TIF1γ) and TRIM66 (TIF1δ);
the Group VIII BRD is selected from speckled protein (SP) family proteins SP100, SP110, SP140 and SP140L;
the Group VIII BRD is selected from transcriptional corepressors ZMYND8 and ZMYDN11; and
the Group IX is selected from WD-repeat proteins BRWD1 (WDR9), BRWD3 and PHIP (WDRT1).

110. The compound of claim 108, wherein DDP is a BRD selected from PCAF, GCN5L2, p300/CBP, TAFT, TAF1L, BRPF1A/B (BR140), BRPF2 (BRD1) BRPF3, BRD8 (SMAP), chromatin remodeling factors SMARCA2 (BRM), SMARCA4 (BRG1), BRD7, BRD9, PBRM1 (polybromo), BAZ1A (ACF1), BAZ1B (WSTF, William-Beuren syndrome transcription factor), BAZ2A, BAZ2B, BPTF, CECR2, ATAD2, ATAD2B, Tripartite-motif-containing (TRIM) family proteins TRIM24 (TIF1α), TRIM28 (TIF1β, KAP1), TRIM33 (TIF1γ), TRIM66 (TIF1δ), WD-repeat proteins BRWD1 (WDR9), BRWD3 and PHIP (WDR11).

111. A stable ternary complex comprising:
a. CBP/p300;
b. a disease protein (DP); and
c. heterobifunctional conditional inhibitor compound of Formula (II): 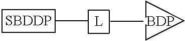
wherein:
SB-CBP/p300 is a silent binder of CBP/p300;
L is an optional linker; and
BDP is a binder of a disease protein (DP);
wherein CBP/p300 and DP are present in a cell of interest (COI) and the relative abundance of the DP in the COI is greater than the relative abundance of CBP/p300 in the COI.

112. The stable ternary complex of any one of claims 105-111, wherein DP is a nuclear hormone receptor protein, G-protein coupled receptor, epidermal growth factor receptor, RAS protein, or transcription factor.

113. The stable ternary complex of any one of claims 105-111, wherein DP is androgen receptor (AR), estrogen receptor (ER), progesterone receptor (PR), glucocorticoid receptor (GR), mineralocorticoid receptor (MR), angiotensin receptor, apelin receptor, bombesin receptor, bradykinin receptor, calcitonin receptor, chemokine receptor, cholecytokinin receptor, corticotropic-releasing factor receptor, galanin receptor, ghrelin receptor, glucagon receptor, glycoprotein hormone receptor, gonadotropin-releasing hormone receptor, kisspeptin receptor, melanocortin receptor, motilin receptor, neuromedin U receptor, neuropeptide FF/AF receptor, neuropeptide S receptor, neuropeptide W/B receptor, neuropeptide Y receptor, opioid receptor, orexin receptor, parathyroid hormone receptor, prokineticin receptor, prolactin-releasing peptide receptor, QRFP receptor, relaxin family peptide receptor, somatostatin receptor, tachykinin receptor, thyrotropin-releasing hormone receptor, urotensin receptor, vasopressin, oxytocin receptor, vasoactive intestinal peptide receptor, PACAP receptor, HER1, HER2, HER3, HER4, KRAS, KRAS-G12C, HRAS, NRAS, or BCL6.

114. The compound of any one of claims 105-111, wherein the DP is androgen receptor (AR), estrogen receptor (ER), progesterone receptor (PR), glucocorticoid receptor (GR), mineralocorticoid receptor (MR), retinoic acid receptor-related orphan nuclear receptor γ (RORγ), apelin receptor, bombesin receptor, bradykinin receptor, chemokine receptor, cholecytokinin receptor, chymase, gonadotropin-releasing hormone receptor, indoleamine 2,3-dioxygenase 1 (IDO1), kisspeptin receptor, neuromedin U receptor, neuropeptide FF/AF receptor, neuropeptide S receptor, neuropeptide W/B receptor, neuropeptide Y receptor, somatostatin receptor, tachykinin receptor, HER1, HER2, HER3, HER4, KRAS, KRAS-G12C, HRAS, NRAS, or BCL6.

115. The stable ternary complex of any one of claims 105-111, wherein DP is androgen receptor (AR) or estrogen receptor (ER).

116. The stable ternary complex of any one of claims 105-111, wherein DP is androgen receptor (AR).

117. The stable ternary complex of any one of claims 105-111, wherein DP is androgen receptor (AR) or DP is estrogen receptor (ER) and the heterobifunctional conditional inhibitor compound is the compound of any one of claims 64-88 or 104.

118. A stable ternary complex comprising:
a. CBP/p300;
b. Androgen receptor (AR) or Estrogen receptor (ER); and
c. heterobifunctional conditional inhibitor compound of any one of claims 64-88 or 104;
wherein CBP/p300 and DP are present in a cell of interest (COI) and the relative abundance of the DP in the COI is greater than the relative abundance of CBP/p300 in the COI.

119. A method of selectively inhibiting the activity of a disease-dependent protein (DDP) in a cell of interest (COI) of a mammal comprising administering a heterobifunctional compound of any one of claims 1-104, or a pharmaceutically acceptable salt or solvate thereof, wherein the heterobifunctional compound inhibits the activity of the DDP in the COI but does not inhibit the activity of the DDP in cells expressing the DDP and not expressing the DP.

120. The method of claim 119, wherein the DP is overexpressed, overactive or both overexpressed and overactive in the COI.

121. The method of claim 119, wherein DP is a nuclear hormone receptor protein, G-protein coupled receptor, epidermal growth factor receptor, RAS protein, or transcription factor.

122. The method of claim 119 or claim 120, wherein DP is androgen receptor (AR), estrogen receptor (ER), progesterone receptor (PR), glucocorticoid receptor (GR), mineralocorticoid receptor (MR), angiotensin receptor, apelin receptor, bombesin receptor, bradykinin receptor, calcitonin receptor, chemokine receptor, cholecytokinin receptor, corticotropic-releasing factor receptor, galanin receptor, ghrelin receptor, glucagon receptor, glycoprotein hormone receptor, gonadotropin-releasing hormone receptor, kisspeptin receptor, melanocortin receptor, motilin receptor, neuromedin U receptor, neuropeptide FF/AF receptor, neuropeptide S receptor, neuropeptide W/B receptor, neuropeptide Y receptor, opioid receptor, orexin receptor, parathyroid hormone receptor, prokineticin receptor, prolactin-releasing peptide receptor, QRFP receptor, relaxin family peptide receptor, somatostatin receptor, tachykinin receptor, thyrotropin-releasing hormone receptor, urotensin receptor, vasopressin, oxytocin receptor, vasoactive intestinal peptide receptor, PACAP receptor, HER1, HER2, HER3, HER4, KRAS, KRAS-G12C, HRAS, NRAS, or BCL6.

123. The method of claim 119 or claim 120, wherein the DP is androgen receptor (AR), estrogen receptor (ER), progesterone receptor (PR), glucocorticoid receptor (GR), mineralocorticoid receptor (MR), retinoic acid receptor-related orphan nuclear receptor γ (RORγ), apelin receptor, bombesin receptor, bradykinin receptor, chemokine receptor, cholecytokinin receptor, chymase, gonadotropin-releasing hormone receptor, indoleamine 2,3-dioxygenase 1 (IDO1), kisspeptin receptor, neuromedin U receptor, neuropeptide FF/AF receptor, neuropeptide S receptor, neuropeptide W/B receptor, neuropeptide Y receptor, somatostatin receptor, tachykinin receptor, HER1, HER2, HER3, HER4, KRAS, KRAS-G12C, HRAS, NRAS, or BCL6.

124. The method of claim 119 or claim 120, wherein DP is AR or ER.

125. The method of claim 119 or claim 120, wherein DP is androgen receptor (AR).

126. The method of any one of claims 118-125, wherein DDP is Ataxia-telangiectasia mutated (ATM), Ataxia telangiectasia and Rad3-related protein (ATR), Aurora Kinase A (AurkA), AurkB, Cell division cycle 7-related protein kinase (CDC7), Checkpoint kinase 1 (CHK1), CHK2, Cyclin-dependent kinase 1 (CDK1), CDK2, CDK4, CDK5, CDK6, CDK9, DNA methyltransferase 1 (DNMT1), Exportin 1 (XPO1), Histone deacetylase 1 (HDAC1), HDAC2, HDAC3, kinesin family member 11 (KIF11), Mitogen-activated protein kinase kinase 1 (MEK1), MEK2, Myc, neuronal precursor cell-expressed developmentally down-regulated protein 8 (NEDD8), SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), Protein arginine methyltransferase 5 (PRMT5), splicing factor 3b subunit 1 (SF3B1), WEE1, 20S proteasome subunits, Steroid Receptor Coactivator 1 (SRC1), SRC2, or SRC3.

127. The method of any one of claims 118-125, wherein DDP is Aurora Kinase A (AurkA), Checkpoint kinase 1 (CHK1), CHK2, CDK4, CDK6, Myc, SMARCA2/4, CREB-binding protein (CBP)/p300, WD repeat-containing protein 5 (WDR5), DDB1- and CUL4-associated factor 1 (DCAF1), phosphatidylinositol-3 kinase (PI3K), or WEE1.

128. The method of any one of claims 118-125, wherein DDP is a BRD selected from PCAF, GCN5L2, p300/CBP, TAF1, TAF1L, BRPF1A/B (BR140), BRPF2 (BRD1) BRPF3, BRD8 (SMAP), chromatin remodeling factors SMARCA2 (BRM), SMARCA4 (BRG1), BRD7, BRD9, PBRM1 (polybromo), BAZ1A (ACF1), BAZ1B (WSTF, William-Beuren syndrome transcription factor), BAZ2A, BAZ2B, BPTF, CECR2, ATAD2, ATAD2B, Tripartite-motif-containing (TRIM) family proteins TRIM24 (TIF1α), TRIM28 (TIF1β, KAP1), TRIM33 (TIF1γ), TRIM66 (TIF1δ), WD-repeat proteins BRWD1 (WDR9), BRWD3 and PHIP (WDR11).

129. The method of any one of claims 118-125, wherein the DDP is CREB-binding protein (CBP)/p300.

130. A method of treating cancer in a mammal comprising administering a therapeutically effective amount of to the mammal a heterobifunctional compound of any one of claims 1-104, or a pharmaceutically acceptable salt or solvate thereof.

131. The method of claim 130, wherein the cancer is a hormone dependent cancer.

132. The method of claim 130, wherein the cancer is adenomas and neoplasms of the prostate, tumor cells expressing the androgen receptor, tumor cells expressing the estrogen receptor, prostate cancer, breast cancer, endometrial cancer, or uterine cancer.

133. A pharmaceutical composition comprising a compound of any one of claims 1-104, or a pharmaceutically acceptable salt or solvate thereof, and at least one pharmaceutically acceptable excipient.
